(19) United States
(12) Patent Application Publication
(10) Pub. No.: US 2003/0170683 A1
(43) Pub. Date: Sep. 11, 2003

(54) Formin-2 nucleic acids and polypeptides and uses thereof

(76) Inventors: Philip Leder, Chestnut Hill, MA (US); Benjamin Leader, Boston, MA (US)

Correspondence Address: CLARK & ELBING LLP, 101 FEDERAL STREET, BOSTON, MA, 02110, US

(21) Appl. No.: 10/308,485

(22) Filed: Dec. 3, 2002

Related U.S. Application Data
(63) Continuation of application No. 09/835,232, filed on Apr. 12, 2001, now abandoned.
(60) Provisional application No. 60/196,811, filed on Apr. 13, 2000.

(51) Int. Cl. C12Q 1/68; G01N 33/53; G01N 33/567

(52) U.S. Cl. 435/6; 435/7.2

(57) ABSTRACT

The invention features methods and reagents for diagnosing and treating recurrent pregnancy loss by using formin-2 nucleic acids and polypeptides that have been identified to play a role in oocyte development.

### CROSS-REFERENCE TO RELATED APPLICATIONS

This application is a continuation of U.S. Utility application Ser. No. 09/835,232, filed Apr. 12, 2001 (now pending), and claims benefit from U.S. Provisional application serial No. 60/196,811, filed Apr. 13, 2000 (now abandoned), each of which is hereby incorporated by reference.

### BACKGROUND OF THE INVENTION

Recurrent pregnancy loss (RPL), also known as recurrent spontaneous abortion or miscarriage, is currently defined as two or more miscarriages, and can be an emotionally devastating medical condition. RPL has been estimated to occur in 1% of pregnant women. There are numerous causes of RPL, including anatomic, endocrine, and immunologic factors. However, the most common cause of early fetal demise is chromosomal abnormalities of the fetus itself which include trisomies (22, 21, 15, 14, 18, and 13) and triploidy (69 chromosomes). Currently, the exact mechanism leading to these chromosomal abnormalities is unknown, but a commonly held belief is they arise from problems with the oocyte spindle formation and meiotic division.

Formins

The Formin-1 gene (Fmn-1), encoded by the limb deformity (ld) locus, is required for proper limb development in mice and has been the subject of much study since its discovery. Mice homozygous for mutations in Fmn-1, in addition to having kidney defects, display multiple limb abnormalities such as fusion of the long bones (e.g. ulna/radius, tibia/fibula) and syndactyly (fusion of digits). This phenotype is caused by the inability of the limb bud mesenchyme to appropriately stimulate or maintain the apical ectodermal ridge of the limb bud, leading to improper expression of FGF-4 and Shh in the limb bud.

Although the requirement for Fmn-1 has been well described at the organismal and tissue level, a definitive role for Fmn-1 at the cellular level has not been clearly identified. This is partly due to the complexity of the Fmn-1 gene. The 400 kb ld locus, originally identified by the fortuitous insertion into the ld locus of a transgene, produces a number of differentially spliced variants of Fmn-1. These different isoforms have partially overlapping expression patterns that make targeted disruptions of the Fmn-1 gene difficult to interpret. Although mutations in the 3′ end of Fmn-1 have been shown to result in limb deformity phenotypes, targeted deletion analysis of variably-spliced N-terminal exons fail to produce a limb phenotype. These data suggest that redundancy may exist among the Fmn-1 proteins since the carboxy terminus is shared among the different isoforms, while the amino terminus exons are isoform specific.

Genetically, Fmn-1 has been shown to interact with two genes to control proper limb formation. The Strong's luxoid (lst) mutation, which causes polydactyly (extra digits) due to ectopic polarizing activity, was shown to be suppressed by a mutant Fmn-1 allele. The lst gene was later identified as the Aristaless-like4 (Alx4) gene, which encodes a paired-type homeodomain protein. Recently, the Gli3 gene, mutations in which also cause polydactyly, was shown to interact synergistically with Fmn-1 to position the Shh signaling center to the posterior limb-bud margin.

Attempts to elucidate the true cellular interactors of the Fmn-1 protein have met with limited success. First, the subcellular site of Fmn-1 activity is currently unclear. On the one hand, evidence exists that Fmn-1 is localized in the nucleus and that some ld mutations result in the retention of Fmn-1 in the cytoplasm. Fmn-1 has also been shown to bind DNA cellulose and to be modified posttranslationally by phosphorylation of serine and threonine residues. On the other band, one report demonstrates that a fraction of chicken formin protein can interact with c-Src at cell membranes when both are overexpressed in COS cells. A phage expression screen with the proline rich region from Fmn-1 identified SH3 domain containing proteins and a number of novel proteins, named the formin binding proteins (FBPs), which contain a proline-binding WW domain. The FBPs bind specifically to certain proline residues, and FBP21 has been implicated in RNA splicing. However, physiological evidence of an association between Fmn-1 and any of the SH3 or WW domain-containing proteins is still lacking.

Although the cellular interactors of Fmn-1 remain uncertain, the Fmn-1 gene is the founding member of a rapidly growing family of genes that encode the formin homology (FH) proteins. FH proteins have been defined by two formin homology domains, FH1 and FH2, which appear together in FH proteins. The FH1 domain, characterized by a proline-rich region, is located approximately in the middle of each FH protein. The FH2 domain was originally identified as a 71 amino acid stretch of high similarity in the carboxy terminal half of FH proteins. By allowing lower levels of homology, recent reports have included the entire C-terminus of FH proteins when defining the FH2 domain. An FH3 domain has also been described which consists of two to three small islands of low similarity located between the N-terminus and the beginning of the FH1 domain.

FH proteins, for the most part, play a role in cytoskeletal organization and/or establishment of cell polarity. There are at least four subfamilies of FH proteins that have been described to date. Three of these have begun to be characterized at the functional level. The S. cerevisiae gene, BNI1, defines one subfamily, and two Drosophila genes, cappuccino and diaphanous, define two other subfamilies. Most recently, BNI1 has been shown to organize microtubules by mediating spindle positioning and movement in the budding process. BNI1 and another S. cerevisiae formin, BNR1, have also been linked to rho G-proteins and to the control of actin organization via profilin binding. Certain mutations in the diaphanous gene cause defects in cytokinesis of germline cells that lead to sterility in both male and female Drosophila. Mutations in one human diaphanous homologue were found in a patient with sterility due to ovarian failure, indicating a possible conservation of sequence and function at the organismal level between humans and flies. However, another human homologue was discovered as the gene, DFNA1, which is responsible for nonsyndromic deafness. The DFNA1 gene is proposed to be a Rho target and profilin ligand that regulates actin organization in the cytoskeleton of hair cells in the inner ear, the same cellular role proposed for its murine orthologue, p140mDia. Mutations in the Drosophila gene, cappuccino, a maternal-effect fertility factor that defines the third formin subfamily, cause Drosophila females to produce embryos with have disorganized microtubules and lack proper anteroposterior and dorsoventral patterning as a result of failure to properly position mRNAs. Until now, the cappuccino subfamily only included Drosophila cappuccino, mouse Fmn-1 (isoforms Ia, Ib, II, III, and IV), and the chicken orthologue of Fmn-1, isoform IV.

### SUMMARY OF THE INVENTION

The invention features methods and reagents for diagnosing and treating recurrent pregnancy loss.

In a first aspect method for determining whether a patient has an increased risk for recurrent pregnancy loss, the method including the step of determining whether the patient has a mutation in Fmn-2, wherein a mutation indicates that the patient has an increased risk for recurrent pregnancy loss.

In a second, related aspect, the invention features a method for determining whether a patient has an increased risk for recurrent pregnancy loss. In this method, the determination includes measuring Fmn-2 biological activity in the patient or in a cell from the patient, wherein decreased levels in the Fmn-2 biological activity, relative to normal levels, indicates that the patient has an increased risk for recurrent pregnancy loss.

In yet another related aspect, the invention features a method for determining whether a patient has an increased risk for recurrent pregnancy loss by measuring Fmn-2 expression in the patient or in a cell from the patient, wherein decreased levels in the Fmn-2 expression relative to normal levels, indicates that the patient has an increased risk for recurrent pregnancy loss. Fmn-2 expression can be determined by measuring levels of Fmn-2 polypeptide or Fmn-2 RNA.

In still another related aspect, the invention features a method for determining whether a person has an altered risk for recurrent pregnancy loss by examining the person's Fmn-2 gene for polymorphisms, wherein the presence of a polymorphism associated with recurrent pregnancy loss indicates the person has an altered risk for recurrent pregnancy loss.

In another aspect, the invention features a substantially pure Fmn-2 polypeptide (e.g., human Fmn-2 or mouse Fmn-2).

In still another aspect, the invention features a substantially pure nucleic acid molecule that hybridizes at high stringency conditions to human Fmn-2 and encodes a polypeptide having Fmn-2 biological activity. In one embodiment, the nucleic acid molecule is human Fmn-2. The nucleic acid can be, for example, DNA or RNA.

In yet another aspect, the invention also features an expression vector that includes a nucleic acid molecule that hybridizes at high stringency conditions to human Fmn-2 and encodes a polypeptide having Fmn-2 biological activity.

The invention also features a cell expressing the expression vector of the previous aspect.

In still another aspect, the invention features a method of treating a human having recurrent pregnancy loss or being at risk for the recurrent pregnancy risk by administering to an oocyte of the human a Fmn-2 polypeptide.

In a related aspect, the invention features a method of treating a human having recurrent pregnancy loss or being at risk for the recurrent pregnancy risk by administering to an oocyte of the human a nucleic acid molecule encoding a Fmn-2 polypeptide. In one example, the nucleic acid molecule is human Fmn-2 RNA.

In another related aspect, the invention features a method of preventing recurrent pregnancy loss in a human by administering to an oocyte of the human an expression vector comprising a Fmn-2 nucleic acid molecule operably linked to a promoter, wherein the promoter is operative in the oocyte.

In still another related aspect, the invention features a method of preventing or ameliorating the effects of a disease-causing mutation in a Fmn-2 gene in a human by introducing into the human an expression vector that includes a promoter operably linked to a Fmn-2 nucleic acid molecule.

The invention also features a method of increasing Fmn-2 biological activity in an oocyte of a human by administering to the oocyte a nucleic acid molecule that hybridizes at high stringency conditions to human Fmn-2 and encodes a polypeptide having Fmn-2 biological activity. In one embodiment, the human has recurrent pregnancy loss or is at risk for the recurrent pregnancy risk.

In still another aspect, the invention features a transgenic mouse in which one or both alleles of the Fmn-2 gene have been mutated such that Fmn-2 biological activity is reduced (e.g., by 25% or more) or absent. The mutation can be introduced by, for example, insertional mutagenesis, homologous recombination, or by introducing one or more point mutations.

In another aspect, the invention features a pharmaceutical composition that includes (i) a nucleic acid molecule encoding human Fmn-2; and (ii) a pharmaceutically acceptable carrier.

In another aspect, the invention features a pharmaceutical composition that includes (i) human Fmn-2; and (ii) a pharmaceutically acceptable carrier.

By “formin-2” or “Fmn-2” is meant a nucleic acid or polypeptide that is substantially identical to the mouse or human Fmn-2 cDNA or Fmn-2 polypeptide. Preferably, the nucleic acid shares at least 70% identity with mouse or human Fmn-2 over a stretch of 50 consecutive nucleotides, more preferably at least 80%, and more preferably at least 90% or even 95% identity. Gaps of up to 10% may be included in one or both of the sequences. Preferably, the polypeptide shares at least 80% identity with mouse or human Fmn-2 over a stretch of 25 consecutive amino acids, more preferably at least 80%, and more preferably at least 90% or even 95% identity. Again, gaps of up to 10% may be included in one or both of the sequences.

By “Fmn-2 biological activity” is the ability to essentially replace human or mouse Fmn-2 in a biological assay. One preferred Fmn-2 biological activity the ability to correct the polyploidy in embryos of Fmn-2^{-/-} mice, as described herein.

By “recurrent pregnancy loss” is meant the occurrence of two or more spontaneous abortions in a person.

By “increased risk recurrent pregnancy loss” is meant that the likelihood for two or more spontaneous abortions is at least twice as high as it is for an average woman of the same age and ethnicity.

By “high stringency conditions” is meant hybridization in 2×SSC at 40° C. with a DNA probe length of at least 40 nucleotides. For other definitions of high stringency conditions, see F. Ausubel et al., Current Protocols in Molecular Biology, pp. 6.3.1-6.3.6, John Wiley & Sons, New York, N.Y., 1994, hereby incorporated by reference.

By “pharmaceutically acceptable carrier” means a carrier, diluent, or excipient that is physiologically acceptable to the treated mammal while retaining the therapeutic properties of the compound with which it is administered. One exemplary pharmaceutically acceptable carrier is physiological saline.

By “substantially identical” is meant a polypeptide or nucleic acid exhibiting at least 50%, preferably 85%, more preferably 90%, and most preferably 95% identity to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “substantially pure polypeptide” is meant a polypeptide that has been separated from the components that naturally accompany it. Typically, the polypeptide is substantially pure when it is at least 60%, by weight, free from the proteins and naturally-occurring organic molecules with which it is naturally associated. Preferably, the polypeptide is a Fmn-2 polypeptide that is at least 75%, more preferably at least 90%, and most preferably at least 99%, by weight, pure. A substantially pure Fmn-2 polypeptide may be obtained, for example, by extraction from a natural source, by expression of a recombinant nucleic acid encoding a Fmn-2 polypeptide, or by chemically synthesizing the protein. Purity can be measured by any appropriate method, e.g., by column chromatography, polyacrylamide gel electrophoresis, or HPLC analysis.

A protein is substantially free of naturally associated components when it is separated from those contaminants that accompany it in its natural state. Thus, a protein that is chemically synthesized or produced in a cellular system different from the cell from which it naturally originates will be substantially free from its naturally associated components. Accordingly, substantially pure polypeptides not only include those derived from eukaryotic organisms but also those synthesized in E. coli or other prokaryotes.

By “substantially pure DNA” is meant DNA that is free of the genes which, in the naturally-occurring genome of the organism from which the DNA of the invention is derived, flank the gene. The term therefore includes, for example, a recombinant DNA which is incorporated into a vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences. It also includes a recombinant DNA that is part of a hybrid gene encoding additional polypeptide sequence.

By “a decrease” is meant a lowering in the level of biological activity, as measured by a lowering/increasing of: a) protein, as measured by ELISA; b) reporter gene activity, of at least 30%, as measured by reporter gene assay, for example, lacZ/β-galactosidase, green fluorescent protein, luciferase, etc.; c) mRNA, levels of at least 30%, as measured by PCR relative to an internal control, for example, a “housekeeping” gene product such as β-actin or glyceraldehyde 3-phosphate dehydrogenase (GAPDH). In all cases, the lowering is preferably by 30%, more preferably by 40%, and even more preferably by 70%.

By “protein” or “polypeptide” or “polypeptide fragment” is meant any chain of more than two amino acids, regardless of post-translational modification (e.g., glycosylation or phosphorylation), constituting all or part of a naturally-occurring polypeptide or peptide, or constituting a non-naturally occurring polypeptide or peptide.

Other features and advantages will be apparent from the following detailed description of the preferred embodiments.

### BRIEF DESCRIPTION OF THE DRAWINGS

FIGS. 1A-1C show the amino acid sequence and cloning of Fmn-2. (A) The structure of Fmn-2 (1567 amino acid putative open reading frame, GenBank accession number AF218940) is depicted including 5′ region, proline rich FH1 domain, and the FH2 domain (SEQ ID NO: 1). Two human ESTs encoding human Fmn-2 sequence are also shown. Overlapping cDNA clones from different sources are shown including one clone from a 17.5 d embryo library, two clones from a mouse brain library, four race clones, and four confirmatory PCR clones. Asterisk marks where brain clone 2 differs from brain clone 4 by containing an extra repeat motif. The proline repeat motif of the FH1 domain is shown and numbered 1 through 11 with every other repeat underlined in purple for clarity. Repeats −2, −1, and 12 through 15 contain alterations and are underlined in black. (B) Cartoon of sequence comparisons between Fmn-2 and other formin family members. The numbers represent % identity and % similarity to Fmn-2 at the amino acid level. (C) Alignment of the C-terminal (FH2) region 3′ of the FH1 domain of cappuccino subfamily members. Alignment generated using the DNASTAR program Megalign and the Clustal program. Shading in black denotes identity between two or more amino acids. Shading in gray denotes similarity of two or more amino acids.

FIG. 2 shows that Fmn-2 maps to the distal end of mouse Chromosome 1. Map from the Jackson BSB backcross showing part of Chromosome 1. The map is depicted with the centromere toward the top. A 3 cM scale bar is shown to the right of the figure. Loci mapping to the same position are listed in alphabetical order. Raw data from The Jackson Laboratory were obtained from the World Wide Web address http://www.jax.org/resources/documents/cmdata.

FIGS. 3A and 3B show that Fmn-2 is expressed in mouse and human brain. (A) Northern blot analysis of polyA selected mouse RNA (˜2 μg/lane) using Probe B (3′ UTR; FIG. 1A) shows expression of an approximately 6.6 Kb transcript in E12.5 whole embryo and adult brain (two day exposure). Faint expression is seen in E9.5 whole embryo, and an additional transcript is detected (<1.5 Kb) in testis. As a loading control, a probe to GAPDH was used with a five hour exposure. (B) A 1.9 Kb cDNA from the 3′ end of human Fmn-2 (FIG. 1A) was used to probe a Clontech multitissue human mRNA dot blot. Strong expression is seen in all tissues of the adult central nervous system (A1-B6), including the pituitary gland (D4) and spinal cord (B7). Fetal brain (G1) also showed heavy expression. Lower level expression was detected in multiple other tissues (colon (C4), uterus (C6), prostate (C7), stomach (C8), testis (D1), ovary (D2), adrenal gland (D5), kidney (E1), small intestine (E3), appendix (F1), fetal kidney (G3), and fetal lung (G7). Abbreviations: E12.5, embryonic day 12.5 whole embryo; E9.5, embryonic day 9.5 whole embryo; brain—cb, brain without cerebellum; GAPDH, glyceradehyde-3-phosphate dehydrogenase; mam. gland, mammary gland; sk. muscle, skeletal muscle;

FIGS. 4A-4L show Fmn-2 expression in the developing central nervous system. Using whole mount in situ hybridization with an antisense riboprobe derived from Probe B (3′ UTR; FIG. 1A), no expression was detected at E8.5 (A) or E9.0 (B). Low level, diffuse expression begins at E9.5 with higher expression in the spinal cord, developing brain structures, and otic vesicle (C). Expression continues and intensifies in the telencephalon, mesencephalon, and rhombencephalon during E10.0 (D) and E10.5 (E). Dorsal (F) and ventral (G) views of an E10.0 embryo (positioned next to a glass bead) demonstrate preferential expression in the spinal cord, rhombic lip, and telencephalon. (H) View of E10.5 revealing strong expression telencephalon and mesencephalon. (I) E9.5 using sense control probe. (J) Continued expression in developing brain structures is shown by sagittal section of E12.5. (K) Staining in transverse sections of E10.5 embryos confirms neural tube expression (arrowhead) and reveals dorsal root ganglion expression (arrow). (L) Preferential expression in rhombencephalon, mesencephalon, and telencephalon demonstrated in sagittal section of E10.5. Nearly identical expression patterns in E10.5 sagittal and transverse sections were observed using Probe A (5′ UTR; FIG. 1A). Abbreviations: me, mesencephalon; nt, neural tube; ov, otic vesicle; rh, rhombencephalon; te, telencephalon; vt, ventricular zone. Scale bars: 1 mm (J), (L); 100 μm (K).

FIGS. 5A-5H show in situ detection of Fmn-2 mRNA in P2 mouse brain. Section in situ was performed on P2 mouse brain using an antisense probe derived from Probe B (3′ UTR; FIG. 1A) which detected the same expression pattern as probe A (5′ UTR; FIG. 1A). (A) Sagittal section. Expression detected in olfactory bulb, cortex, and cerebellum in addition to diffuse, low level expression throughout the brain. The black line marks the approximate location of the coronal section pictured in (B). (B) Coronal section. Expression is detected in the cortex and hippocampus. Scattered, punctate expression is detected in the thalamus and hypothalamus. (C) Magnified view of the olfactory bulb (white box in (A)) Staining is detected in the internal granular layer and mitral layer but not in the external plexiform layer or in the glomerular cells. (D) Magnified view of the region contained in the smaller box in (D) demonstrates expression in the granular layer of the dentate gyrus and the CA3 field. (E) Magnified view of the cerebellum (black box (A)) showing expression in granular layer. (F) Magnified view of the region contained in the large box in (B) shows scattered, punctate expression in the thalamus, with no hybridization in the habenulopeduncular tracts. (G) Pax6 control showing expression in cerebellum and olfactory bulb. (H) Magnified view of boxed region in (G) showing heavy expression of Pax6 in the cerebellar external granular layer and fainter expression in the internal granular layer. Abbreviations: C, cortex; Cb, cerebellum; CA3, CA3 field of the hippocampus; EGL, external granular layer of cerebellum; EP, external plexiform layer of olfactory bulb; Gl, glomerular layer of olfactory bulb; GrDG, granular layer, dentate gyrus; H, hypothalamus; Hb, habenulopeduncular tracts; Hi, hippocampus; IGL, internal granular layer of olfactory bulb; Mi, mitral layer of olfactory bulb; Ob, olfactory bulb; T, thalamus. Scale bars: 1 mm (A), (B), (G); 100 μm (C), (D), (E), (F), (H).

FIGS. 6A-6F show expression of Fmn-2 in adult mouse brain. Section in situ hybridizations were performed using an antisense probe generated from Probe B (3′ UTR; FIG. 1A) which detected the same expression pattern as with an antisense probe generated from Probe A (5′ UTR; FIG. 1 A). (A) Diffuse hybridization in cortex with no expression detected in white matter. Arrowhead points to dense hybridization in piriform cortex. (B) Magnified view of white boxed region in (A) demonstrates the dense hybridization in cortex region and no hybridization in the white matter. (C) Heavy, diffuse expression seen throughout cortex especially the piriform cortex (arrowhead). Specific expression was seen in CA1 and CA3 fields of the hippocampus and in the granular layer of the dentate gyrus. Very little to no hybridization was detected in the thalamus. (D) Further magnification of the region contained in the white box in (C) shows areas of Fmn-2 expression in the cortex and the CA1 field of the hippocampus (arrowhead). Fmn-2 is not expressed in the white matter. (E) Expression of Fmn-2 seen in the Purkinje cell layer of the cerebellum with additional punctate hybridization is detected in the medullary brain stem. (F) Increased magnification of the region contained in the white box in (E) shows the dense hybridization in Purkinje cells (arrowhead). Low level, diffuse expression is seen in the molecular layer and the granular layer. No hybridization is detected in the branching white matter of the cerebellum (white arrow head). Abbreviations: co, cortex; wm, white matter; gr, granular layer; me, medulla; mo, molecular layer; th, thalamus. Scale bars: 1 mm (A, C, E); 100 μm (B, D, F).

FIGS. 7A-7C show targeting of the mouse Fmn-2 gene in ES cells and generation of Fmn-2 deficient mice. (A) Homologous recombination by the targeting vector deletes a 1300 bp of the proline exon (which encodes 433 amino acids of the proline-rich FH1 domain) and replaces this sequence with 1257 bp containing the PGK-Neo gene followed by stop codons in all three reading frames. (B) Southern blot analysis of genomic DNA derived from 11 offspring born from Fmn2^{+/-} (heterozygous) matings. Genomic DNA (˜5 μg) was digested with PvuII and probed using a 5′ external probe. (C) Northern blot analysis of total brain RNA from Fmn2^{+/+}, Fmn2^{+/-} and Fmn2^{-/-} mice using a probe to the 3′ UTR of Fmn-2 and GAPDH as an RNA loading control. Fmn-2^{+/+}, wildtype; Fmn-2^{+/-}, Fmn-2 heterozygote; Fmn-2^{-/-}, Fmn-2 homozygote.

FIGS. 8A-8D show that embryos from Fmn-2^{-/-} females exhibit heterogeneous morphological alterations. Decidua at embryonic day 9.5 (E9.5) were isolated from Fmn-2^{+/+} and Fmn-2^{-/-} female mice all bred with Fmn-2^{+/+} male mice. Ten decidua (A) and contents (C) at approximately E9.5 isolated from a Fmn-2^{+/+} female. Nine E9.5 decidua (B) and their contents (D) isolated from a Fmn-2^{-/-} female. The decidua from the Fmn-2^{-/-} female contained presumably genetically identical embryos which exhibited significant differences in embryonic development: Three decidua contained no visible embryonic tissue, Five contained embryos at various stages of developmental arrest, and one contained an apparently normal embryo.

FIGS. 9A-9H show that Fmn-2 expression is restricted to the oocyte in the ovarian compartment. Ovaries were collected from eCG-stimulated Fmn-2^{+/+}, Fmn-2^{+/-}, and Fmn-2^{-/-} mice and labeled with a ³⁵S-Fmn2 antisense probe. Darkfield (left panel) and brightfield (right panel) photomicrographs are shown at 40× (A, B, E-H) or 100× (C, D). (A, B) Fmn-2 transcripts are observed throughout folliculogenesis and can be observed in both primary follicles (asterisk) and Graafian follicles (arrow). (C, D) A cumulus enclosed oocyte in a large Graafian follicle is shown at higher magnification. (E, F) Fmn-2 transcripts are observed in ovary sections from Fmn-2^{+/-} mice. (G, H) Labeling was not observed in ovarian sections from Fmn-2^{-/-} mice.

FIGS. 10A-10J show cytoskeletal analysis of oocytes from Fmn-2^{+/+}, Fmn-2^{+/-} and Fmn-2^{-/-} mice. Actin, (red, A, C, E, G, I), tubulin (green, B, D, F, H, I) and chromosome (blue, B, D, F, H, I) organization were examined by fluorescence microscopy. (A-D) Fmn-2^{+/+} and Fmn-2^{-/-} mice were induced to superovulate with PMS and hCG. Ovulated oocytes were collected from oviducts 14-18 hours after hCG injection. Oocytes from Fmn-2^{+/+} mice have completed meiotic maturation are arrested at metaphase of Meiosis II and exhibit normal actin (A), tubulin (B, green) and chromosome (B, blue) organization, consistent with previous reports. Oocytes from Fmn-2^{-/-} mice have a uniform cortical actin array (C) with no signs of polar body formation consistent with Metaphase I arrest. These oocytes contained a centrally located spindle with two distinct patterns of tubulin and chromosome staining: (1) oocytes with a barrel-shaped spindle and chromosomes aligned on the metaphase plate (D-right) or (2) oocytes at late anaphase with an elongated spindle and a set of chromosomes associated with each spindle pole (D-left). E, F Three oocytes from Fmn-2^{-/-} mice injected with Fmn-2 mRNA have formed a polar body. One polar body is close to normal in appearance (E, F bottom) and two have large polar bodies with small, spherical actin structures next to it (E, F top). (G-J) GV stage oocytes were matured in vitro for eight hours and then treated with nocodazole for two hours. An oocyte from a Fmn-2^{+/-} mouse has chromatin located at the cortex with actin organized around it (G) and a nocodazole-disrupted spindle (H). An oocyte from a Fmn-2^{-/-} mouse has chromatin located in the center of the oocyte with no evidence of actin organization (I) despite a disrupted spindle (J). (scale bar=40 μm)

FIGS. 11A-11G show that a meiotic delay at metaphase of Meiosis I and subsequent fertilization results in embryonic polyploidy. (A) During normal meiotic maturation, oocytes are ovulated at metaphase of Meiosis II with an anchored spindle of 2n chromosome content (oocyte 1.3). Fertilization results in the formation of the second polar body (oocyte 1.5). Oocytes arrested at metaphase of Meiosis I (oocytes 2.3a, 2.4a) at the time of fertilization, would complete Meiosis I, give off a polar body and then enter interphase and initiate embryonic development resulting in triploidy (oocyte 2.5a). Oocytes at late anaphase of Meiosis I at the time of fertilization (oocytes 2.3b, 2.4b), would enter interphase, condense the two sets of chromosomes at either pole and the sperm nucleus, fuse and then initiate embryonic development, resulting in a pentaploid karyotype. Cytogenetic analysis of blastocysts (3.5d) resulting from the mating of a Fmn-2^{-/-} female and a Fmn-2^{+/+} male revealed diploid (e, 2n=40), triploid (f, 3n=60) or pentaploid (g, Sn=100) metaphase spreads. This mechanism was supported using immunofluorescence and dual-labeled micrographs of tubulin (green) and chromatin (blue) are shown (B-D). The fertilized egg isolated from a Fmn-2^{+/+} mouse (B) contains the condensed male (B, M) pronucleus and the female (B, F) pronucleus with multiple nucleoli, a condensed chromatin mass is also observed in the polar body (B, pb). The fertilized, presumably triploid egg (C) from a Fmn-2^{-/-} female contains the male pronucleus (C, M) and more intense female pronucleus (C, F), consistent with a larger chromosome number (2N), and a single polar body (C, pb). The fertilized, presumably pentaploid, egg from the same Fmn-2^{-/-} female contains two female pronuclei with multiple nucleoli, the right one more condensed than the left (D, F) and a single male pronucleus (D, M). White scale bar: 30 μm; black scale bar: 12.5 μm

FIG. 12A shows the mouse Fmn-2 amino acid sequence (SEQ ID NO: 2).

FIG. 12B shows the mouse Fmn-2 cDNA sequence (SEQ ID NO: 3).

FIG. 12C shows the sequence of the human 5′ EST encoding Fmn-2 (SEQ ID NO: 4).

FIG. 12 D shows the sequence of the human 3′ EST encoding Fmn-2 (SEQ ID NO: 5).

FIGS. 13A (SEQ ID NO: 6) and 13B (SEQ ID NO: 7) show the shotgun sequences of two human BACs containing Fmn-2 genomic sequence.

FIG. 14 is a schematic illustration showing the location of human Fmn-2 exonic sequence identified in the two human ESTs and two human BACs.

### DETAILED DESCRIPTION

We have discovered murine and human formin-2 (Fmn-2), a gene that bears a high degree of similarity to Fmn-1 and cappuccino. The mouse gene, which encodes a putative 1567 amino acid open reading frame and maps to mouse Chromosome 1, is expressed almost exclusively in the developing and mature central nervous system. Expression begins at embryonic day (E) 9.5 in the developing spinal cord and brain structures and continues in neonatal and adult brain structures including the olfactory bulb, cortex, thalamus, hypothalamus, hippocampus and cerebellum. Human Fmn-2 has a similar expression pattern.

We have also discovered that Fmn-2 is required for proper control of meiosis in the mammalian egg. In the mouse, when part of this gene is deleted, 97-100% of oocytes are unable to complete meiosis correctly. These oocytes are then ovulated and fertilized, leading to the production of triploid and pentaploid fetuses that spontaneously abort. Since the molecular role of Fmn-2 appears to have been conserved in yeast, fruit flies, and mice, it is likely that Fmn-2 will function similarly in humans. Thus, one can identify the cause of recurrent pregnancy loss by detecting mutations in Fmn-2.

In addition, evidence in the mouse suggests that mutations in Fmn-2 lead to the meiotic spindle detaching from the oocyte periphery, a result found to occur in aging human oocytes. The occurrence of trisomies, such as Trisomy 21 (which causes Down's Syndrome), is believed to be linked to this process. Thus, it may be possible that down regulation of or accumulated mutations in Fmn-2 may also be a direct cause of aging in human oocytes and can be used as a predictor of producing a child with Down's Syndrome. This would lead to use of genetic testing of Fmn-2 in women of advanced maternal age considering bearing children, an increasingly common situation.

We have also discovered that the Fmn-2 protein is expressed in the oocyte. The human oocyte is one of the largest cells in the human body and can be directly fertilized by injection of sperm. A direct method of therapy can be imagined in which Fmn-2 protein or Fmn-2 mRNA can be directly injected into oocytes with defective copies of the Fmn-2 gene, thereby allowing the oocytes to progress normally through meiosis. This would provide a therapy for women with mutations in the Fmn-2 gene without making germline modifications of their future embryos.

Detection of Formin-2 Mutations and Altered Expression Levels

Fmn-2 polypeptides and nucleic acid sequences find diagnostic use in the detection or monitoring of conditions involving recurrent pregnancy loss. For example, mutations in Fmn-2 that decrease Fmn-2 biological activity may be correlated with recurrent pregnancy loss in humans. Accordingly, a decrease or increase in the level of Fmn-2 production may provide an indication of a deleterious condition. Levels of Fmn-2 expression may be assayed by any standard technique. For example, the regulatory sequences may be assayed as a means of determining whether altered expression is likely, or Fmn-2 transcription may be quantified. Fmn-2 expression in a biological sample (e.g., a biopsy) may be monitored by standard northern blot analysis or may be aided by PCR.

A biological sample obtained from a patient may be analyzed for one or more mutations in Fmn-2 nucleic acid sequences using a mismatch detection approach. Generally, these techniques involve PCR amplification of nucleic acid from the patient sample, followed by identification of the mutation (i.e., mismatch) by either altered hybridization, aberrant electrophoretic gel migration, binding or cleavage mediated by mismatch binding proteins, or direct nucleic acid sequencing. Any of these techniques may be used to facilitate mutant Fmn-2 detection, and each is well known in the art; examples of particular techniques are described, without limitation, in Orita et al. (Proc. Natl. Acad. Sci. USA 86:2766-2770, 1989) and Sheffield et al. (Proc. Natl. Acad. Sci. USA 86:232-236, 1989). The Fmn-2 diagnostic assays described above may be carried out using any biological sample in which Fmn-2 is normally expressed. ldentification of a mutant Fmn-2 gene may also be assayed using these sources for test samples.

Alternatively, a Fmn-2 mutation, particularly as part of a diagnosis for predisposition to Fmn-2-associated recurrent pregnancy loss, may be tested using a DNA sample from any cell, for example, by mismatch detection techniques. Preferably, the DNA sample is subjected to PCR amplification prior to analysis.

In yet another approach, immunoassays are used to detect or monitor Fmn-2 protein expression in a biological sample. Fmn-2-specific polyclonal or monoclonal antibodies may be used in any standard immunoassay format (e.g., ELISA, western blot, or RIA) to measure Fmn-2 polypeptide levels. These levels would be compared to wild-type Fmn-2 levels. Immunohistochemical techniques may also be utilized for Fmn-2 detection. For example, a tissue or cell sample may be obtained from a patient, sectioned, and stained for the presence of Fmn-2 using an anti-Fmn-2 antibody and any standard detection system (e.g., one which includes a secondary antibody conjugated to horseradish peroxidase).

In one preferred example, a combined diagnostic method may be employed that includes an evaluation of Fmn-2 protein production and also includes a nucleic acid-based detection technique designed to identify more subtle Fmn-2 mutations (for example, point mutations). As described above, a number of mismatch detection assays are available to those skilled in the art, and any preferred technique may be used. Mutations in Fmn-2 may be detected that either result in loss of Fmn-2 expression or loss of normal Fmn-2 biological activity.

Therapies

Therapies may be designed to circumvent or overcome a Fmn-2 gene defect or inadequate Fmn-2 gene expression, and thus modulate and possibly alleviate conditions involving Fmn-2 loss. Fmn-2 is expressed in the oocytes. Hence, in considering various therapies, it is understood that such therapies will be targeted to the oocyte. Reagents that modulate Fmn-2 biological activity may include, without limitation, full length Fmn-2 polypeptides, or fragments thereof, Fmn-2 mRNA or antisense RNA, or any compound which modulates Fmn-2 biological activity, expression, or stability.

Protein Therapy

Treatment or prevention of recurrent pregnancy loss can be accomplished by replacing mutant Fmn-2 protein with normal protein, or by altering the levels of normal or mutant protein. It is also be possible to modify the pathophysiologic pathway in which the protein participates in order to correct the physiological defect.

To replace a mutant protein with normal protein, or to add protein to cells that no longer express sufficient Fmn-2, it is desirable to obtain large amounts of pure Fmn-2 protein from cultured cell systems that can express the protein. Delivery of the protein to the oocyte can then be accomplished using appropriate packaging or administrating systems. Alternatively, small molecule analogs may be used and administered to act as Fmn-2 agonists and in this manner produce a desired physiological effect.

Gene Therapy

Gene therapy is another potential therapeutic approach in which normal copies of the Fmn-2 gene or nucleic acid encoding Fmn-2 antisense RNA are introduced into oocytes to successfully encode for normal and abundant protein or Fmn-2 antisense RNA in cells which express excessive normal or mutant Fmn-2, respectively.

Administration of Fmn-2 Polypeptides, Fmn-2 Genes, or Modulators of Fmn-2 Synthesis or Function

A Fmn-2 protein, gene, or modulator of Fmn-2 may be administered within a pharmaceutically-acceptable diluent, carrier, or excipient, in unit dosage form. Any appropriate route of administration may be employed. One such method is direct injection into the oocyte.

The following examples are to illustrate the invention. They are not meant to limit the invention in any way.

### EXAMPLE 1

Isolation and Cloning of Fmn-2

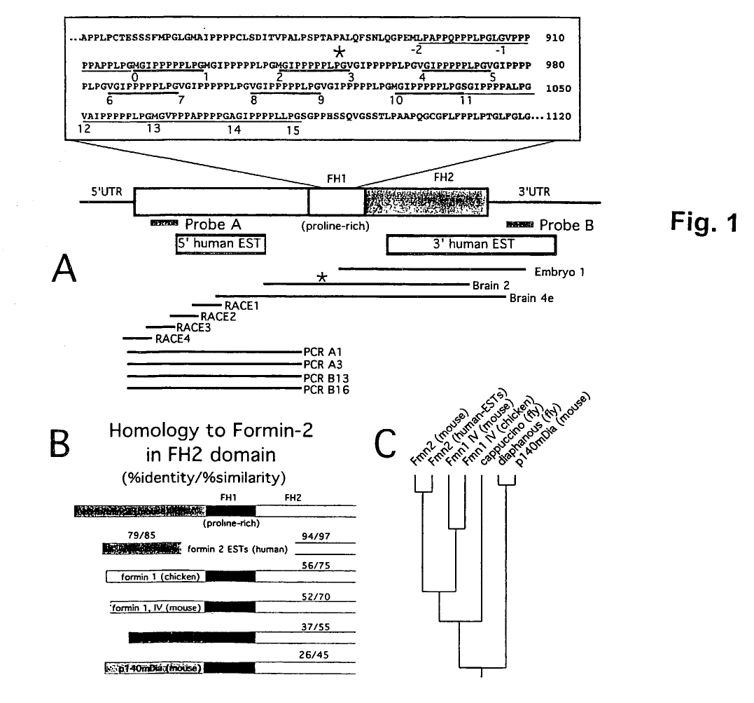

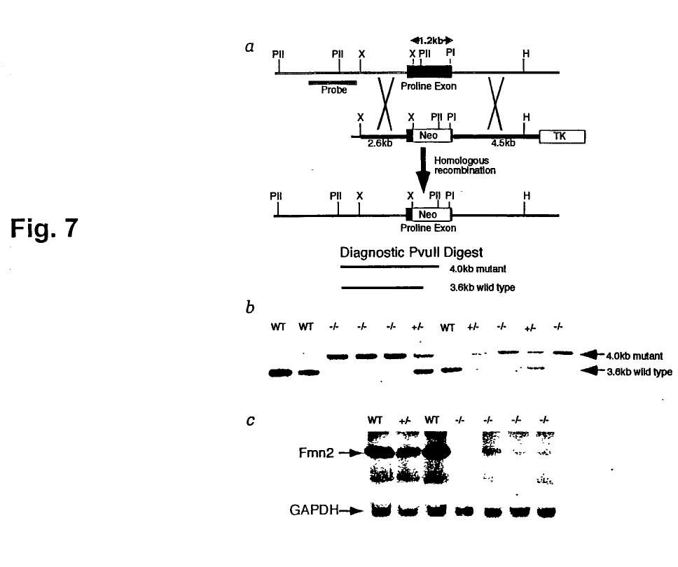

To search for Fmn-1 homologues we used a human EST having high similarity to Fmn-1 in a low stringency, murine cDNA library screen. The screen identified a novel cDNA, which we named Fmn-2. To obtain the full open reading frame we performed additional library screens followed by 5′ RACE and conventional PCR (FIG. 1A). The deduced open reading frame of Fmn-2 encodes 1567 amino acids and a predicted protein of 166 kDa (GenBank accession number AF218940; FIG. 7D). Using the mouse Fmn-2 sequence, an additional human EST with similarity in the N-terminal region was identified using the BLASTP program and sequenced (FIG. 1A). Human and mouse Fmn-2 share 90/93% identity/similarity over the C-terminus (294 amino acids from partial human Fmn-2 sequence) and 79/85% identity/similarity over most of the N-terminus (using 331 amino acids from partial human Fmn-2 sequence) (FIG. 1B).

Further database searches using the BLASTP program revealed Fmn-2 to be a formin homology (FH) protein. Fmn-2 contains both the proline-rich FH1 and the FH2 domains characteristic of FH proteins and is closely related to Fmn-1 with 55/72% identity/similarity over the entire C-terminus of the two genes (FIG. 1B) and 37/42% identity/similarity in the FH1 domain. Fmn-2 is also highly similar to Drosophila cappuccino with 37/55% identity/similarity in the C-terminus (FIG. 1B). The high similarity among Fmn-2, cappuccino, and Fmn-1, places mouse and human Fmn-2 in the cappuccino subfamily of the formin homology family of proteins (FIG. 1C).

Despite the large number of formin homology genes identified in the BNI1 and diaphanous subfamilies in the past several years, the size of the cappuccino subfamily has remained constant since the discovery of the cappuccino gene in 1995. Until the discovery of Fmn-2, Fmn-1 was the only potential vertebrate orthologue of cappuccino. Assigning orthologue status between members of the cappuccino subfamily is difficult because, unlike members of the diaphanous subfamily, very little similarity is found among the cappuccino subfamily proteins in the N-terminal domain. For example, chicken Fmn-1 is considered to be the orthologue of mouse Fmn-1, isoform IV because these two proteins share 39% identity in the N-terminus and have similar expression patterns (Trumpp et al., Genes Dev. 6:14-28, 1992). Even less identity would be expected of a Drosophila/murine orthologue relationship. Surprisingly, Fmn-2 contains a 100 amino acid region in the N-terminus (revealed through BLASTP) that is 23/42% identical/similar to cappuccino. Based on sequence alone, however, this low level of similarity is not sufficient for us to conclude anything other than Fmn-2 and Fmn-1 are both potential orthologues of cappuccino. It is clear, though, that mouse and human Fmn-2 are orthologues given 79% N-terminal identity and 94% C-terminal identity in addition to similar expression patterns.

Although all the FH proteins contain proline rich stretches and some have repeat motifs of various lengths, the Fmn-2 FH1 domain possesses the most extensively repeated proline motif yet described in this family. The amino acid sequence (M/V)GIPPPPPLPG (SEQ ID NO: 8) is repeated without alteration 11 times in tandem, followed by an additional four repeats and preceded by an additional two repeats with minor alterations (FIG. 1A). Although we have defined the proline motif as beginning with a methionine or valine and ending with a glycine, the two overlapping brain clones (FIG. 1A) that cover the proline rich region differ by 11 amino acids, GMGIPPPPPLP (SEQ ID NO: 9), which may indicate that this is the repeating unit. The cDNA clone, Brain 2, contained an extra GMGIPPPPPLP (SEQ ID NO: 9) repeat that occurred in the protein sequence in the position marked by the red asterisk (FIG. 1A). When an oligopeptide containing at least one copy of the repeating unit GMGIPPPPPLPGVGIPPPPPLP (SEQ ID NO: 10) was fused to GST, radiolabeled, and then used to probe a western blot of different WW domains, it bound specifically to WW domains of FBP11, FBP2 1, and FBP30, but not to the WW domain of YAP or GST alone, indicating that this motif is capable of binding, to specific WW domains. In addition to binding WW domains, proline rich sequences are known ligands for SH3 domains, EVH1 domains, and the actin binding protein, profilin. Since cappuccino, the closest Drosophila homologue to Fmn-2, and many of the FH proteins have been implicated in profilin binding via the FH1 domain to effect cytoskeletal changes, it is possible that Fmn-2 binds profilin via this repeat. Interestingly, mouse diaphanous, p140mDia, contains a subset of the Fmn-2 repeat, IPPPPPLPG (SEQ ID NO: 11), that is repeated five times exactly with additional repeats that contain modifications of this motif (Watanabe et al., EMBO J 16:3044-3056, 1997).

Searches in Pfam (Bateman et al., Nucl. Acids Res 27:260-262, 1999) using the N-terminus of Fmn-2 did not detect any known domains. At least one of the loosely conserved FH3 domains (Petersen et al., J. Cell Biol. 141: 1217-1228, 1998; Wasserman, Trends Cell Biol. 8:111-115, 1998) was detected although the overall similarity was low. A six amino acid stretch of polyglutamines (five of which were encoded by the CAG trinucleotide repeat) was found in the N-terminus. Additional glutamine doublets were also found throughout the N-terminus. FHOS, a recently cloned human formin homology gene in the diaphanous subfamily, also contained a polyglutamine stretch (Westendorf et al., Gene 232: 173-182, 1999). In addition, Fmn-2 contains numerous potential protein kinase C (PKC) and protein kinase A (PKA) sites.

### EXAMPLE 2

Chromosome Mapping

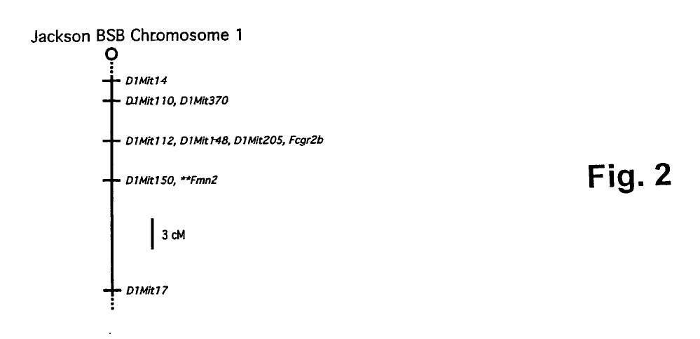

The ld locus which contains the mouse Fmn-1 gene resides on Chromosome 2. We determined the location of the Fmn-2 gene in order to ascertain if Fmn-2 was also located near the ld locus. PCR analysis of the 3′ UTR of Fmn-2 revealed a size and sequence difference in this region between mouse strains C57BL/6J and M. spretus. This difference allowed mapping using single stranded conformational polymorphism (SSCP) with a Jackson Laboratory interspecific backcross (C57BL/6J×M. spretus)F1×C57BL/6J called “Jackson BSB.” No recombinants were detected between Fmn-2 and marker D1Mit150, a marker near the distal end of mouse Chromosome 1 (FIG. 2). The 95% confidence limits for the distance between Fmn-2 and D1Mit150 are 0-3.8 cM. This region shares a homology of synteny with human Chromosome 1 q23-32.

### EXAMPLE 3

Fmn-2 mRNA Expression

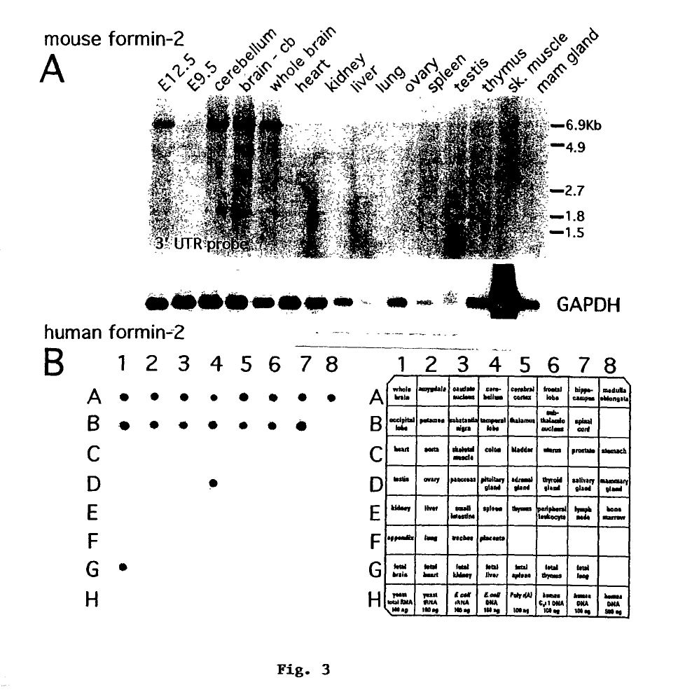

The important developmental role of Fmn-1 led us to investigate the potential role Fmn-2 might play in the developing mouse. To better understand the function of Fmn-2 we first sought to determine the spatial and temporal expression of Fmn-2. A cDNA probe to the 3′ UTR (Probe B, FIG. 1A) of mouse Fmn-2 was used in RNA blot analyses demonstrated that the Fmn-2 gene predominantly encodes one, approximately 6.6 Kb, transcript (FIG. 3A), unlike Fmn-1, which is expressed in several different splice forms. The size and expression of this transcript was confirmed using a probe to the 5′ coding region (Probe A, FIG. 1A). In the adult mouse, this transcript was found primarily in brain tissues. Expression was also detected in E12.5 whole embryo mRNA and, faintly, in E9.5 mRNA. mRNA from testis did, however, contain two smaller transcripts. One faintly-expressed testis transcript, detected by the 3′ probe (Probe B, FIG. 1A), migrated faster than the 1.5 Kb marker (FIG. 3A). The other, detected by the 5′ probe (Probe A, FIG. 1A) migrated between the 1.5 and 1.8 Kb markers.

To determine in which regions of the brain Fmn-2 is expressed and to determine if human Fmn-2 is expressed in a pattern similar to mouse Fmn-2, a human multi-tissue northern blot and RNA dot blot (FIG. 3B) from Clontech were probed using the entire 3′ human EST (FIG. 1A). Human Fmn-2 was detected as an approximately 6-7 Kb transcript (similar in size to mouse Fmn-2). Expression was detected in all central nervous system regions including spinal cord and pituitary gland (FIG. 3B). In addition, fetal brain also showed strong expression (FIG. 3B). Further exposure of the dot blot revealed a second set of tissues having faint expression of the Fmn-2 transcript. Although the level of expression in these tissues was similar to that of the E. coli DNA control, there were still higher than expression in many other tissues (e.g., heart, kidney, etc.) which contained almost no expression, indicating that this low level expression found in some tissues was above background.

### EXAMPLE 4

Fmn-2 Expression During Embryogenesis

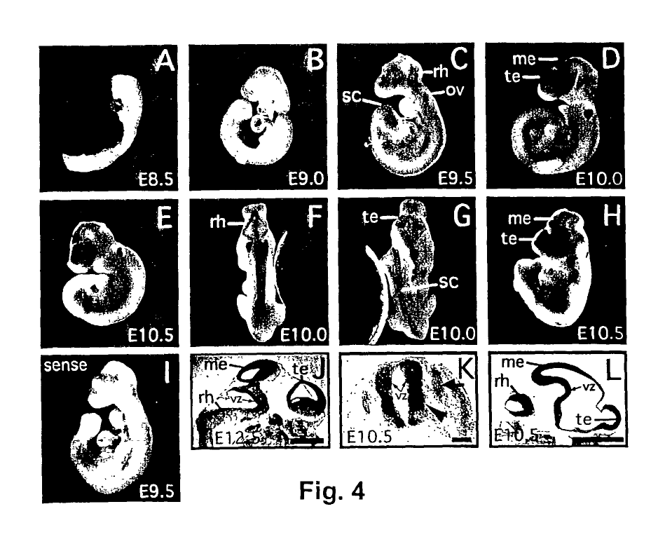

Northern blot analysis suggested that mouse Fmn-2 was expressed as early as E9.5. Dot blot analysis revealed that human Fmn-2 was expressed in fetal brain. In order to determine the spatial and temporal localization of Fmn-2 transcripts during embryological development, whole mount in situ hybridization was performed on E8.5 to E10.5 embryos (FIGS. 4A-H). This analysis demonstrated Fmn-2 expression at E9.5, E10.0, and E10.5. Predominant expression was observed in the spinal cord and developing brain including the telencephalon, mesencephalon, and rhombencephalon, while low-level, diffuse expression was observed throughout the embryo during these stages.

Section in situ hybridization was performed on E10.5 and E12.5 embryos to confirm the staining pattern observed in the whole mounts. In transverse sections of E10.5 embryos, strong expression was again observed in the spinal cord both within and outside of the ventricular zone (FIG. 4K) and in developing brain structures. Fmn-2 expression was also consistently detected in dorsal root ganglia, though the level of expression was weaker than in the spinal cord and was not apparent on whole-mounts (see, e.g., FIGS. 4E and 4G). In sagittal sections, most regions of the developing brain also contained strong Fmn-2 expression within and outside of the ventricular zone at E10.5 (FIG. 4L) and E12.5 (FIG. 4J). Thus, Fmn-2 is preferentially expressed in developing CNS during embryogenesis.

### EXAMPLE 5

Fmn-2 Expression in Adult and Neonatal Brain

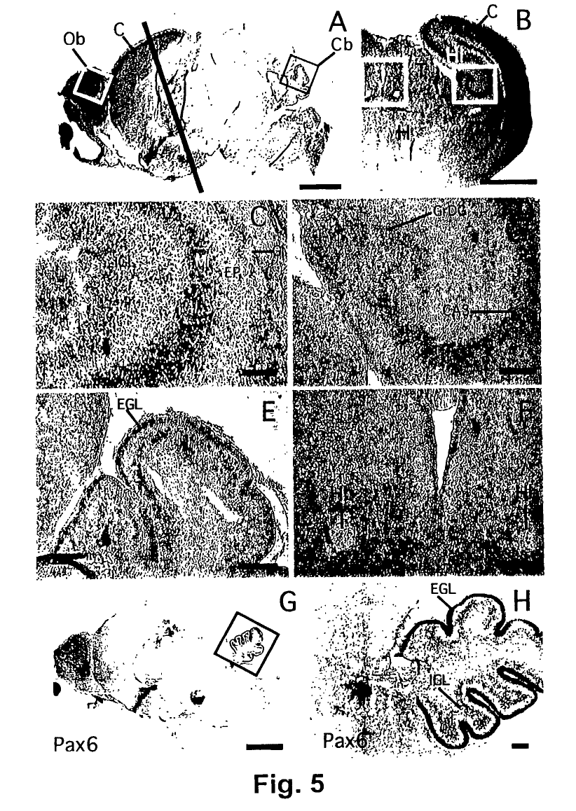

Fmn-2 expression in P2 neonatal mouse brain was analyzed by section in situ hybridization (FIG. 5). Sagittal sections revealed olfactory bulb and cerebellum expression (FIG. 5A). Upon higher magnification, strongest expression in the olfactory bulb can be seen in the internal granular layer (IGL) and mitral layer (Mi), with little to no expression detected in the external plexiform layer (EP) (FIG. 5C). The glomeruli were notably lacking in Fmn-2 expression.

Higher magnification of the cerebellum revealed prominent expression in the external granular layer. In addition, a subset of cells near the Purkinje cell layer also contained Fmn-2 transcripts (FIG. SE). For comparison, Pax 6 shows preferential expression in the external granular layer of the cerebellum and weaker expression throughout the internal granular layer (FIGS. 5G and 5H).

Fmn-2 expression was detected in layers 2-6 of the cerebral cortex (FIG. 5B). In the hippocampus, Fmn-2 was expressed in the granular layer of the dentate gyrus and the pyramidal cells of the CA3 and CA1 fields (FIG. 5D). Under higher magnification, scattered regions of expression were detected in the thalamus (FIG. 5F) and hypothalamus.

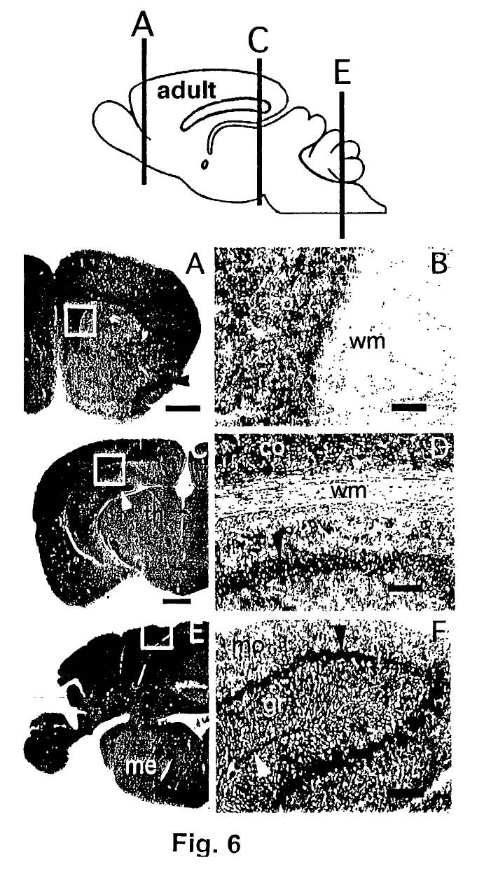

Since each tissue of the adult human brain appeared to contain Fmn-2 transcripts by mRNA dot blot analysis (FIG. 3D), section in situ hybridization was also performed on adult mouse brain to determine which cellular layers of the brain express this gene. In this analysis, Fmn-2 expression was detected throughout the cortex (FIG. 6A). No expression was observed in white matter of the corpus callosum, suggesting higher levels of Fmn-2 expression in neurons than in glia. The neurons of the CA1 (arrowhead, FIG. 6D) and CA3 fields of the hippocampus showed strong expression as did the granular layer of the dentate gyrus (arrowhead, FIG. 6C). Although scattered expression was observed upon high magnification, relatively little expression was detected in the hypothalamus and even less in the thalamus. In the cerebellum, the Purkinje cells (arrowhead in 6F) showed strong expression. Scattered expression was detected in the medulla (FIG. 6E). The expression pattern indicated that Fmn-2 is clearly expressed in many, but not all, neurons of the brain. Since expression was detected in areas of the brain that contained both neurons and glial cells, we cannot exclude the possibility that glial cells may also contain Fmn-2 transcripts.

The similar expression of pattern of mouse and human Fmn-2 in the developing and mature central nervous system suggests a similar role for the human and mouse genes. Interestingly, chicken Fmn-1 is expressed during embryogenesis in the notocord, floor plate, and ventral horns, in addition to the developing limb bud (de la Pompa et al., Dev. Dyn. 204:156-167, 1995). Although mutations in mouse Fmn-1 have not resulted in any neurological defect reported to date, it is also interesting to note that, like Fmn-2, mouse Fmn-1 isoforms I through IV begin to be expressed at the same embryological stage, E9.5 (Chan et al., Development 121: 3151-3162, 1995). Isoforms I-III are expressed in dorsal root ganglia, cranial ganglia, and the developing kidney. Isoform IV is expressed in the notochord, the somites, the apical ectodermal ridge of the developing limb bud, and the developing kidney. Thus, mouse Fmn-1 and Fmn-2 begin expression at the same stage of embryogenesis with partially overlapping expression patterns. It is possible that these two genes may act redundantly in certain tissues of the central nervous system such as dorsal root ganglia. Furthermore, Fmn-2 is expressed in the central nervous system at a time when axonal growth, migration, and synapse formation are occurring. Since these are processes that depend upon dynamic changes in the cytoskeleton and formin homology proteins are thought to be organizers of the cytoskeleton, it is possible that Fmn-2 may function by coordinating cytoskeletal processes during this crucial time.

### EXAMPLE 6

Generation of Mice having a Targeted Deletion of Fmn-2

Mice with targeted deletion of the Fmn-2 gene were generated to define the role of Fmn-2 during mammalian development. A targeted deletion of 433 amino acids of the proline-rich FH1 domain of Fmn-2 was generated using embryonic stem cell technology (FIGS. 7A and 7B). Analyses of Fmn-2^{-/-} mice were performed in 129 inbred mice and in 129/Black Swiss outbred mice, each strain with two lines of mice derived from two, independent Fmn-2-targeted embryonic stem cells. Male and female Fmn-2^{+/-} mice were phenotypically normal and when inter-crossed, produced progeny that did not differ significantly from a normal Mendelian distribution of 1^{+/+}:2^{+/-}:1^{-/-} (45^{+/+}:63^{+/-}:31^{-/-}; P>0.1) or a male ratio of 1:1 (70:64 male:female; P>0.1) indicating no significant selective disadvantages with regard to genotype or sex. Northern analysis confirmed the severe reduction of Fmn-2 message in homozygous mutant mice (FIG. 7C). Although Fmn-2 is expressed in the developing and mature central nervous system, Fmn-2^{-/-} mice showed no gross developmental or central nervous system abnormalities, nor were histological differences noted in brains from Fmn-2^{-/-} mice In a 129/Black Swiss background, fertility appeared normal in Fmn-2^{-/-} males and Fmn-2^{+/-} females, as evidenced by the production of litter sizes similar to those of wildtype females: Fmn-2^{+/+} females mated to Fmn-2^{+/+} males produced an average litter size of 8.9 offspring (n=12 litters) from six mating pairs; Fmn-2^{+/-} females mated to Fmn-2^{-/-} males produced an average litter size of 9.9 offspring (n=15 litters) from nine breading pairs. When mated to either Fmn-2^{+/+} or Fmn-2^{-/-} males, however, Fmn-2^{-/-} females showed drastically reduced fertility (average litter size of 1.6 (n=14 litters) from five breeding pairs). The few mice that were born from Fmn-2^{-/-} females died of neglect, but would survive if fostered.

### EXAMPLE 7

Fmn-2^{-/-} Females Exhibited Recurrent Pregnancy Loss During Early and Midgestational Stages of Development

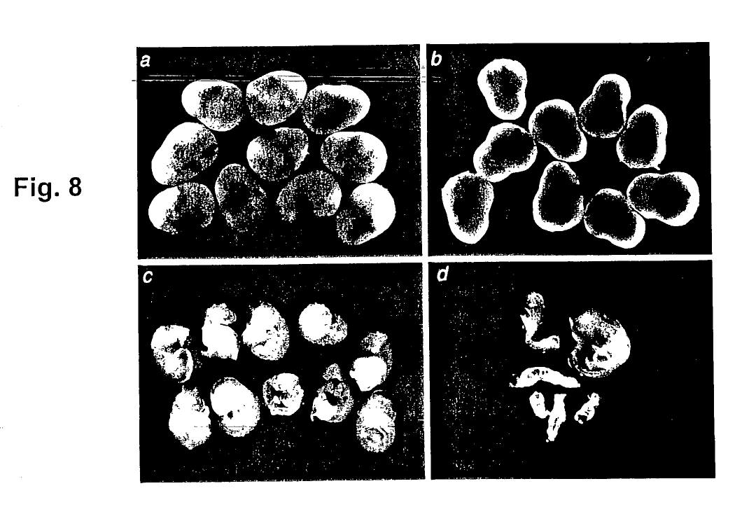

To investigate the mechanism for the observed sub-fertility of Fmn-2^{-/-} females, pregnant Fmn-2^{-/-} or control Fmn-2^{+/+} females mated to Fmn-2^{+/+} males were sacrificed 8.5 and 9.5 days after mating and the uterine decidua dissected. In Fmn-2^{+/+} females, the average number of implantation sites was 11.25 (45 from four females) and most decidua (41/45) contained grossly normal embryos that were at a uniform developmental stage (FIGS. 8A and 8C). When Fmn-2^{-/-} females were mated to Fmn-2^{+/+} males, the uteri contained an average of 10.2 implantation sites (51 from five females), but only 24% of the decidua (7/51) contained grossly normal embryos (FIGS. 8C and 5D). Although all the embryos resulting from the mating of Fmn-2^{-/-} females and Fmn-2^{+/+} males should be genetically identical heterozygotes, striking morphological heterogeneity was observed. The morphologies of these embryos included normal development, developmental delays with gross morphological defects, and in some instances, almost complete resorption with no visible embryonic material (FIGS. 5B and 5D). The abnormal embryos eventually aborted before birth as indicated by the presence of resorption sites in the uteri of pregnant Fmn-2^{-/-} females 16.5-19.5 days post conception (dpc); 54/70 (77%) implantation sites resorbed in uteri from nine females, compared to 16/70 (23%) with phenotypically normal embryos). Thus, Fmn-2^{-/-} females exhibited recurrent pregnancy loss during early and midgestational stages of development.

### EXAMPLE 8

Ovary Transplant Experiments

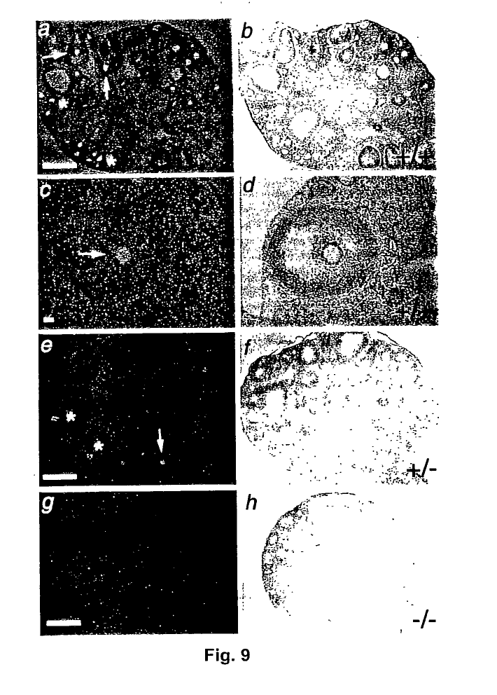

Because the Drosophila homologue of Fmn-2, cappuccino, is expressed in the female fly egg chamber and is required for normal fertility of the female fly (Emmons et al., Genes Dev. 9:2482-2494, 1995), we hypothesized that the defect in Fmn-2^{-/-} female mice might reside in the ovary. To test this notion, an ovary transplant experiment was performed in which the ovaries of Fmn-2^{-/-} 129 females were replaced with ovaries from Fmn-2^{+/+} 129 females. The transplanted Fmn-2^{-/-} females were then mated to Fmn-2^{+/+} males, and the genotype of the offspring confirmed (Fmn-2^{+/+} indicating successful transplantation). An average litter size of 5.4 Fmn-2^{+/+} offspring was obtained from seven litters from four Fmn-2^{-/-} females with transplants of Fmn-2^{+/+} ovaries, compared to 1.6 offspring in untransplanted Fmn-2^{-/-} females. In the reciprocal experiment, Fmn-2^{-/-} ovaries were used to replace the ovaries in five Fmn-2^{+/+} females, none of which gave birth to offspring. The ability to rescue the phenotype by ovarian transplantation indicated that the defect resides in the ovary and was not likely to involve the hypothalamic-pituitary hormonal axis or the ability of the uterus of Fmn-2^{-/-} females to support embryonic development. This result prompted an investigation of Fmn-2 expression in the ovary. The studies of Fmn-2 expression by Northern blot described above were not sufficiently sensitive to detect MRNA in whole ovarian lysates. In contrast, in situ hybridization studies detected an oocyte-specific expression pattern of Fmn-2 in whole ovarian sections. Using a probe to the 3′ UTR, Fmn-2 mRNA was detected exclusively in oocytes (FIGS. 9A-9F). Fmn-2 message was absent in oocytes contained within the resting primordial follicle pool and was first observed in the primary follicle coincident with oocyte growth (FIGS. 9A and 9B). Fmn-2 message was observed in all stages of follicle development including the Graafian stage as illustrated at higher magnification (FIGS. 9C and 9D). Fmn-2 message was not observed in ovarian sections from Fmn-2^{-/-} mice (FIGS. 9G and 9H), confirming that Fmn-2 is not expressed from the targeted allele in detectable levels.

Since the defect in Fmn-2^{-/-} females was ovary-specific and Fmn-2 was expressed in the oocyte, we hypothesized that the recurrent pregnancy loss might be due to defects during oocyte meiotic maturation, a process that includes DNA-spindle positioning and asymmetric cell divisions similar to those observed during bud-formation in S. cerevisiae.

In order to assess meiotic competence of oocytes, oocytes from Fmn-2^{+/+}, Fmn-2^{+/-} and Fmn-2^{-/-} mice were isolated either from oviducts (in vivo) of super ovulated female mice or directly from ovaries (and matured in vitro) (Table I). Oocytes were then processed for immunofluorescence to analyze the organization of tubulin, actin and chromatin (FIGS. 4A-4D). Next, oocytes were scored for the retention of the germinal vesicle (GV), progression to metaphase of Meiosis I (MI) via the germinal vesicle breakdown (GVBD) stage, and progression to metaphase of Meiosis II (MII), which includes the formation of the first polar body (PB) (Table I). Similar percentages of oocytes from Fmn-2^{+/+} and Fmn-2^{+/-} mice proceeded through Meiosis I and extruded the first polar body when matured in vivo (78% MII) or in vitro (82% MII) (Table I). Fmn-2^{+/-} mice were then considered equivalent to Fmn-2^{+/+} mice for later experiments. In contrast, in oocytes from Fmn-2^{-/-} mice, first polar body extrusion was reduced to 11% in in vitro matured oocytes and to 1% in in vivo matured (ovulated) oocytes (Table I).

TABLE I
In vivo and in vitro Meiotic Maturation in Oocytes from Fmn2^{+/+},
Fmn2^{+/}, and Fmn^{2-/-} Mice
Distribution of Oocytes in Meiotic Stages

| | | | | |
|---|---|---|---|---|
| Maternal | | | | |
| Genotype | N | GV/GVBD | PB | Other |

(in vivo maturation)

| | | | | |
|---|---|---|---|---|
| +/+ | 228 | 44 (19.3%) | 178 (78.1%) | 6 (2.6%) |
| −/− | 101 | 92 (93.%) | 3 (3.0%) | 5 (5.0%) |

(in vitro maturation)

| | | | | |
|---|---|---|---|---|
| +/+ | 103 | 20.4% (21) | 77.7% (80) | 1.9% (2) |
| −/− | 136 | 90.4% (123) | 0.7% (1) | 8.8% (12) |

Oocytes which Formed Polar Bodies After Injection

| Injected | N | GVBD | PB | Percent PB |
|---|---|---|---|---|
| −/− | 69 | 63 | 6 | 9% |
| (Water) | | | | |
| −/− | 79 | 44 | 35 | 44% |
| (Fmn2) | | | | |
| +/− | 74 | 35 | 39 | 53% |
| (Fmn2) | | | | |

### EXAMPLE 9

Oocyte Rescue Experiments

In order to determine if Fmn-2 functioned within the oocyte and if a rescue of the phenotype was possible, oocytes from Fmn-2^{-/-} mice were injected with either water or full-length Fmn-2 mRNA. Injection of Fmn-2 mRNA showed a partial rescue of the Fmn-2^{-/-} phenotype in that the rate of first polar body formation was significantly increased from 9% to 44%, a rate not significantly different than that shown by injected wild-type oocytes (53%, Table I).

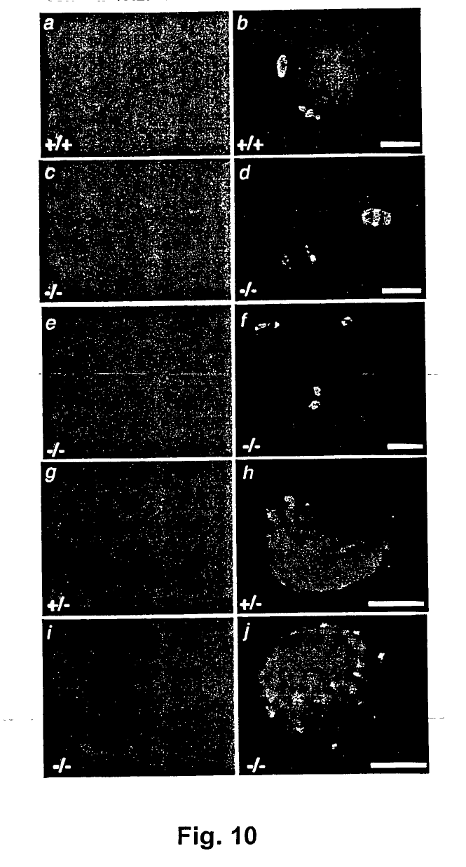

Examination of the cytoskeletal organization of oocytes from Fmn-2^{+/+}, Fmn-2^{+/-}, and Fmn-2^{-/-} mice revealed that oocytes from Fmn-2^{-/-} mice were blocked at metaphase of Meiosis I (FIGS. 10B and 10D). Oocytes contained a centrally located spindle with a uniform cortical actin array (FIG. 10B), a barrel-shaped spindle (FIG. 10B), and chromosomes aligned at the metaphase plate (FIG. 10D). The vast majority of oocytes from Fmn-2^{+/+} and Fmn-2^{+/-} mice were arrested at metaphase of Meiosis II, a finding consistent with previous reports in which a uniform array of cortical actin and an enhanced zone of filamentous actin were observed overlying the meiotic spindle (FIG. 10A). In these mice, meiotic spindles were normal in shape and size and docked at the cortex, and chromosomes were aligned at the metaphase plate (FIG. 10C). A subset of the oocytes from Fmn-2^{-/-} mice appeared to initiate meiotic progression, presumably towards parthenogenic division, as indicated by the two sets of chromosomes observed at both spindle poles in the absence of a polar body and the loss of a barrel-shaped spindle (FIGS. 10B and 10D). The small percentage of oocytes that did manage to move the DNA-spindle apparatus to the cell cortex were able to organize actin and to extrude a polar body, although this was often an abnormally large polar body. Thus, bi-polar spindle formation, chromosome alignment and perhaps even actin organization appear normal in Fmn-2-deficient mice. It appeared, however, that either spindle migration and/or docking to the oocyte cortex was blocked in oocytes from Fmn-2^{-/-} mice, thus preventing further progression through melosis. Injection of Fmn-2 mRNA led to an increase in the frequency of polar body formation when compared to water injection. The polar bodies produced after injection were a mixture of normal looking polar bodies and large polar bodies which were sometimes flanked by small, spherical, actin structures (FIGS. 10D and 10E)

We next sought to determine if the molecular role of Fmn-2 was more likely to be involved in the positioning of chromatin to the cortex of the oocyte or in anchoring of the chromatin at the cortex. It was previously demonstrated that a connection exists between the cortex of the oocyte and metaphase chromatin of meiosis since chromatin will move to the surface of the oocyte once the metaphase spindle is disrupted via nocodazole treatment. As expected, treatment of oocytes from Fmn-2^{+/-} mice with nocodazole for two hours during metaphase of Meiosis I resulted in oocytes with chromatin relocated to the cortex surrounded by localized actin organization 87.5% (21/24 oocytes) of the time (FIGS. 10G and 10H). Two hours after nocodazole treatment, however, only 6% (2/31 oocytes) of Metaphase I oocytes from Fmn-2^{-/-} mice contained chromatin positioned at the cortex with localized actin organization (FIGS. 10I and 10J). This result suggests that Fmn-2 is required for positioning of Metaphase I chromatin to the cortex of the oocyte.

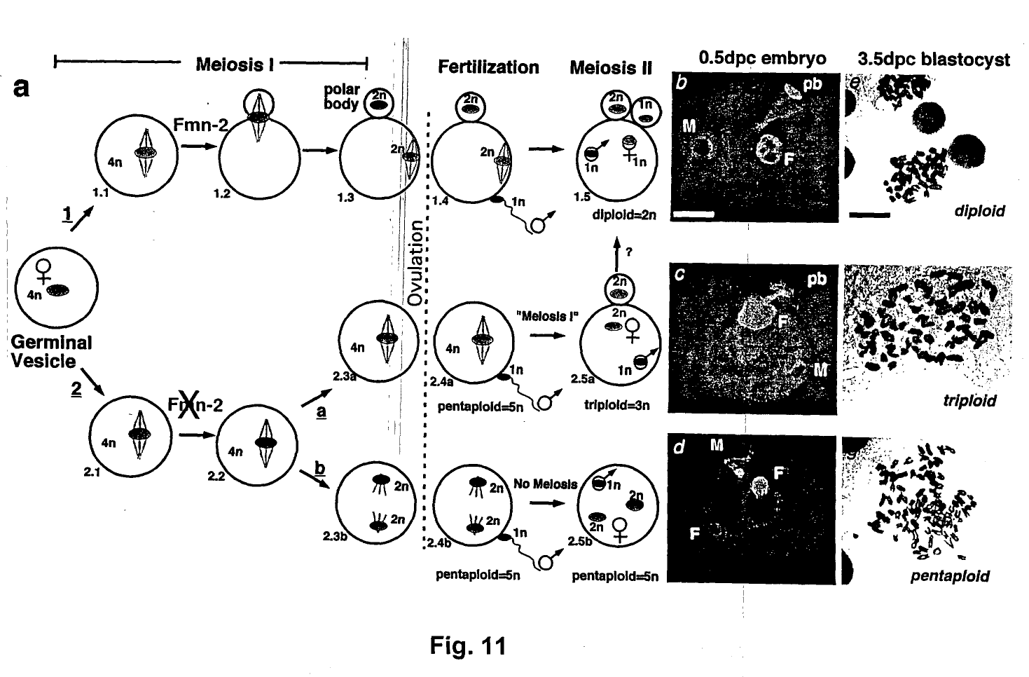

Although Fmn-2^{-/-} mice ovulate oocytes that extrude the first polar body less than 11% (in vitro data) or 1% (in vivo data) of the time, Fmn-2^{-/-} mice produced similar numbers of blastocysts (an average of 7.1 blastocysts from eight females) as compared to Fmn-2^{+/+} females (an average of 6.6 blastocysts from seven females). Furthermore, normal numbers of implantation sites were observed, suggesting that the blastocysts from Fmn-2^{-/-} mice were viable long enough to form a normal decidual response. Despite this, 75% produced embryos that aborted before birth (see above). Previous reports suggest that fertilization of oocytes that do not extrude the first polar body (primary oocytes) leads to the production of triploid embryos which die at various stages of development until midgestation. For example, the LT/Sv strain of mice ovulates two oocyte populations: oocytes arrested at metaphase of Meiosis II and oocytes arrested at metaphase of Meiosis I. Fertilization of oocytes arrested at metaphase of Meiosis I leads to digynic triploidy in which the embryos contain two maternal genomes and one paternal genome, resulting in embryonic lethality. Because oocytes from Fmn-2^{-/-} mice arrest at metaphase of Meiosis I, we can formulate a mechanism to account for the increased embryonic loss observed in Fmn-2-deficient females (FIG. 11). Normally in Meiosis I, oocytes form a metaphase spindle with a tetraploid chromatin content which migrates to the oocyte cortex where it becomes docked and a polar body forms, resulting in an asymmetrical division leaving the oocyte diploid (FIG. 11A-1, oocytes 1.1-1.3). The oocytes then proceed to metaphase of Meiosis II, where they remain arrested until fertilization occurs, inducing the completion of Meiosis II by production of the second polar body (FIG. 11A-1, oocytes 1.4 and 1.5). Mutant oocytes from Fmn-2^{-/-} mice that contain a centrally-located spindle may immediately after fertilization condense their chromatin mass, resulting in a pentaploid embryo after fertilization (FIG. 11A-2, oocyte 2.4a). Alternatively, they may initiate anaphase upon fertilization, complete Meiosis I, extrude the first polar body, and begin embryogenesis as a triploid embryo (FIG. 11A-2, oocyte 2.5a). Mutant oocytes that complete anaphase prior to fertilization might immediately condense their chromatin mass following fertilization, resulting in pentaploid embryos (FIG. 11A-2, oocyte 2.5b).

To test the validity of the model described above, we performed karyotyping of 3.5 dpc blastocysts isolated from Fmn-2^{+/+} or Fmn-2^{-/-} females, each fertilized by Fmn-2^{+/+} males (FIGS. 11E- 11G; Table II). 100% of the blastocysts from Fmn-2^{+/+} females were diploid. In contrast, blastocysts from Fmn-2^{-/-} mice displayed the following distribution: 26% diploid, 50% triploid, and 24% pentaploid (Table II). To exclude the possibility that somatic cell chromosomal abnormalities were contributing to the polyploidy in embryos from Fmn-2-deficient mice, 1.5 day, 2-cell embryos were also karyotyped and revealed a similar distribution (Table II, 29% diploid, 57% triploid, and 14% pentaploid). The percentage of diploid embryos correlated with the number of phenotypically normal embryos (˜25%), just as the percentage of polyploid embryos correlated with the percentage of spontaneously aborted embryos (˜75%). Once born, the diploid embryos develop normally if fostered and are fertile.

To correlate the production of the polyploid embryos with earlier defects in meiosis, tubulin and chromosome organization was examined by immunofluorescence in in vivo fertilized 0.5 d embryos isolated from superovulated Fmn-2^{+/+} and Fmn-2^{-/-} females. Fertilized eggs from Fmn-2^{+/+} females had formed the second polar body and contained male and female pronuclei (FIG. 11B). The majority of fertilized eggs from Fmn-2^{-/-} mice completed polar body formation after fertilization and often contained a female pronucleus that appeared larger and more intensely stained than the male pronucleus (FIG. 11F). In addition, fertilized eggs that contained two female pronuclei and one male pronucleus were observed (FIG. 11G), consistent with the model in which pentaploid embryos are formed by fertilization of Fmn-2^{-/-} eggs that have already divided the tetraploid nucleus without completing cell division (FIGS. 11A-2). It remains unclear as to how a certain percentage of zygotes eventually become diploid after fertilization. It is possible that the rarely-occurring, normal oocytes ovulated by Fmn-2^{-/-} females are fertilized. Since the frequency of these oocytes appears to be, at most, 11% (Table I), however, an additional mechanism is most likely required to form diploid blastocysts 25% of the time. This additional mechanism would require that the tetraploid oocyte bypass or compensate for the delay in metaphase of Meiosis I observed above. Evidence for this type of mechanism has been demonstrated previously when partially-meiotically incompetent oocytes isolated from small antral follicles in the tetraploid state are fertilized in vitro. These incompetent oocytes produce blastocysts of which 75% are digynic triploids and 25% are diploids (Eppig et al., Dev. Biol. 164: 1-9, 1994; Albertini and Eppig, Devel. Genet. 16: 13-19, 1995). Given that fertilization of tetraploid oocytes from both Fmn-2^{+/+} and Fmn-2^{-/-} mice can result in 25% diploid blastocysts, it possible that the ability to correct the ploidy level of the embryo may be Fmn-2-independent.

TABLE II
Cytogenetic Analysis of Blastocysts from Fmn-2^{+/+} and Fmn-2^{-/-} Mice
Distribution of Ploidy in Blastocysts

| Maternal | | | | | |
|---|---|---|---|---|---|
| Genotype | Day | N | Diploid | Triploid | Pentaploid |
| +/+ | 3.5 | 21 | 21(100%) | 0 | 0 |
| −/− | 3.5 | 38 | 10(26%) | 19(50%) | 9(24%) |
| −/− | 1.5 | 21 | 6(29%) | 12(57%) | 3(14%) |

Embryos were flushed from the oviduct or uterus at 1.5d or 3.5d after conception via natural matings and karyotyped using metaphase spreads.
Chromosome counts: Diploid, 2n = 35-40; Triploid, 3n = 50-60, Pentaploid, 5n = 90-100.

### EXAMPLE 10

Human Fmn-2 Sequence

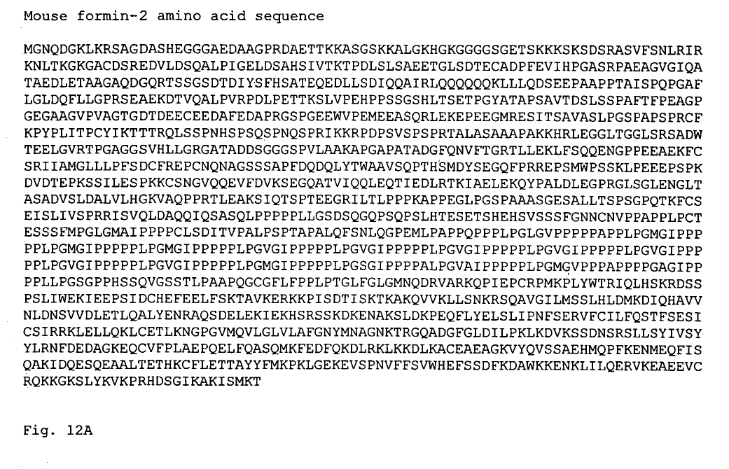

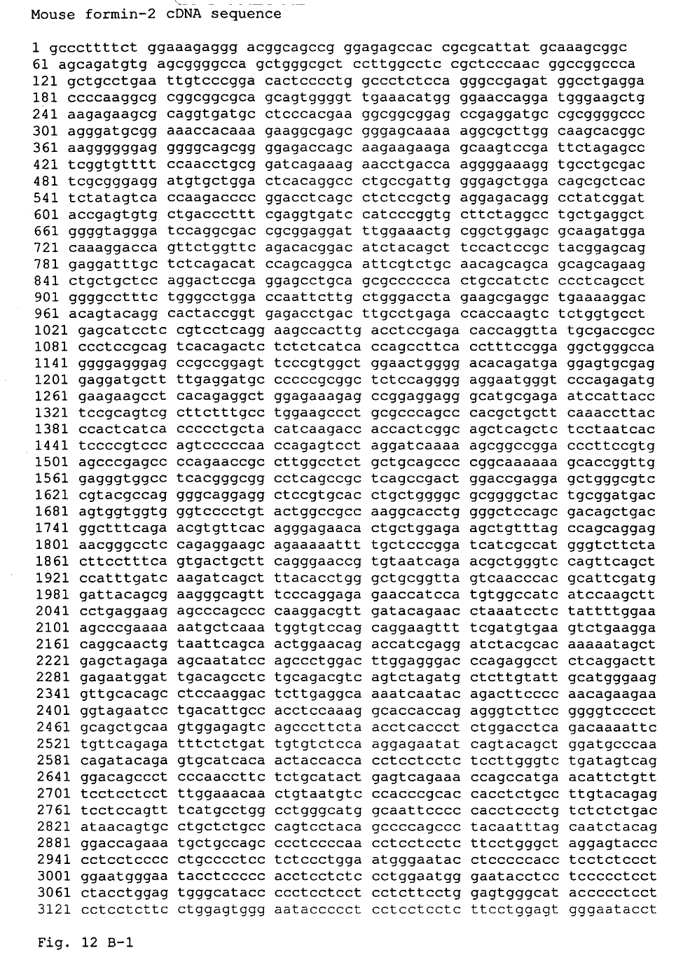

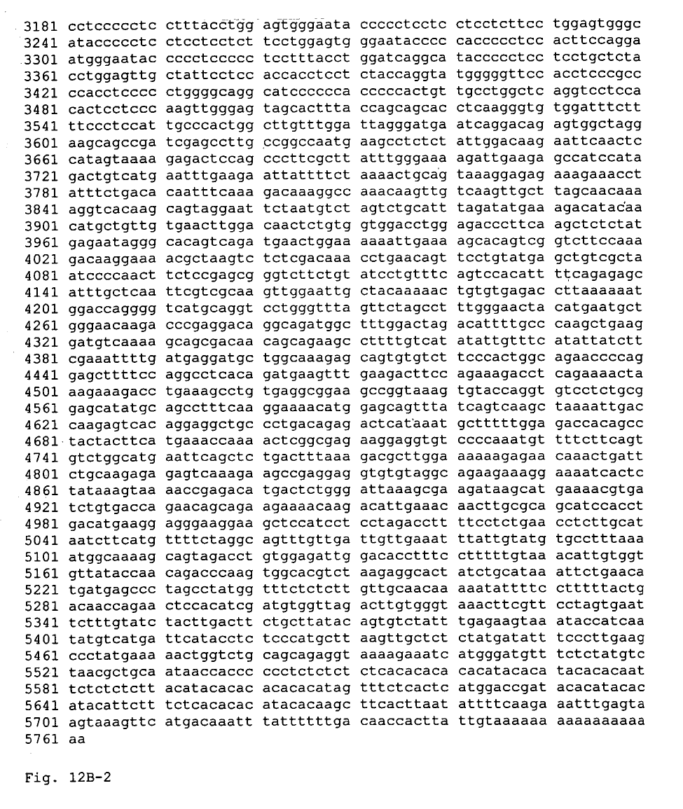

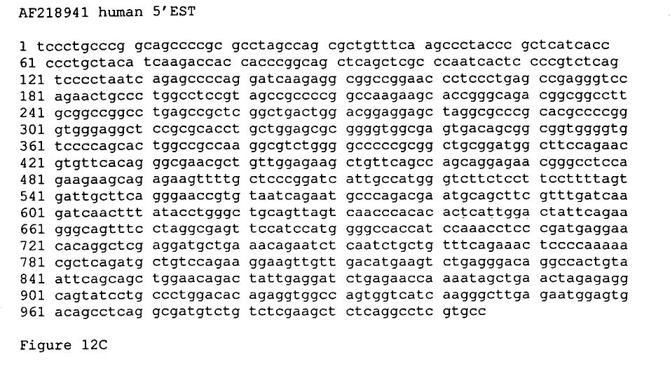

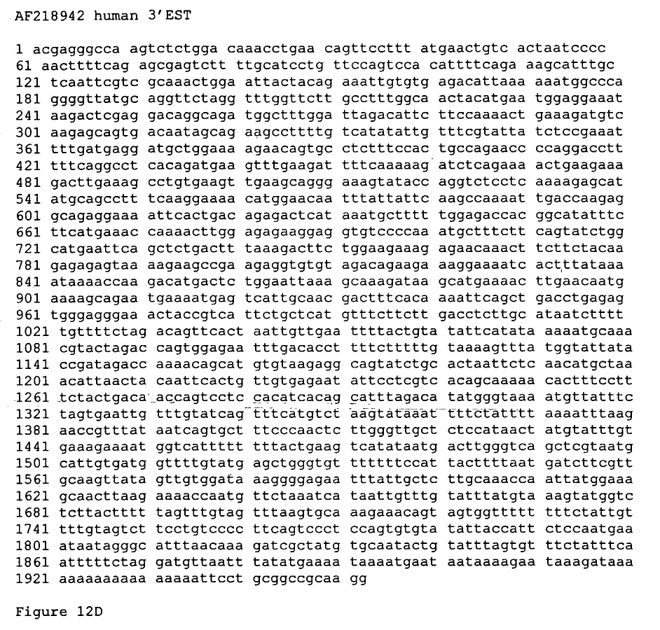

Two human Fmn-2 ESTs were identified in the EST database (5′EST=W39395, 3′EST=r56121). These were sequenced in entirety and the complete sequences were sent to GenBank and were assigned the following GenBank numbers, respectively: AF218941 and AF218942 (FIGS. 12C and 12D). The previously available EST sequences were not of high quality and contained numerous sequence errors that were corrected by double stranded sequencing of the entire EST clones.

### EXAMPLE 11

Sequence of Two Human BACs Containing Fmn-2 Genomic Sequence

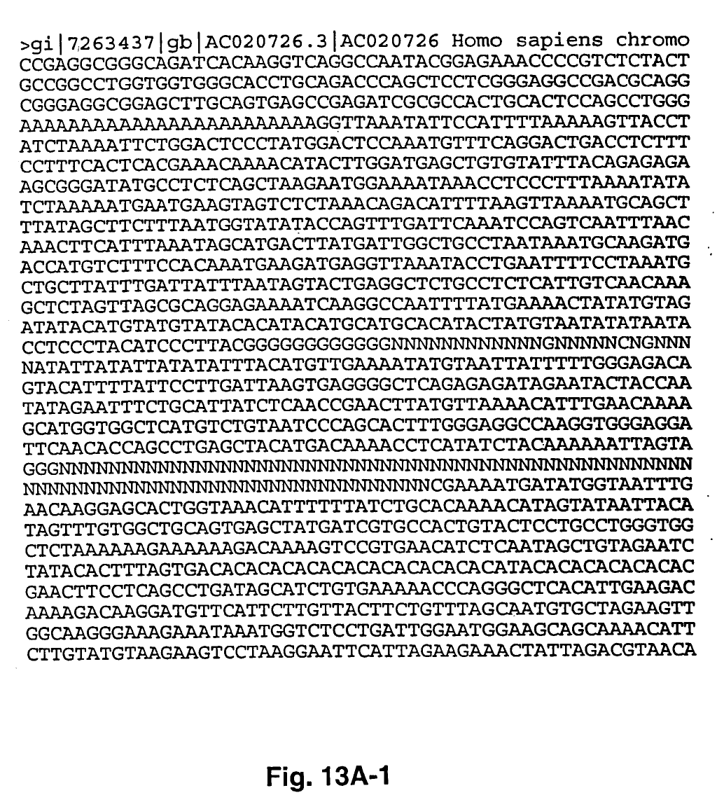

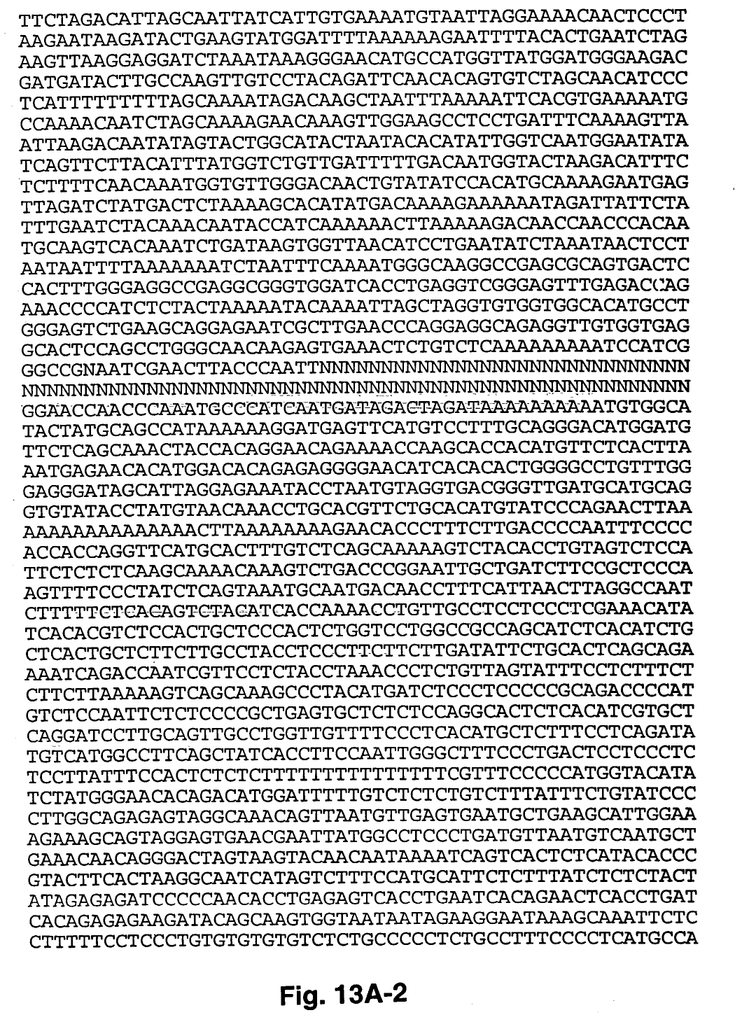

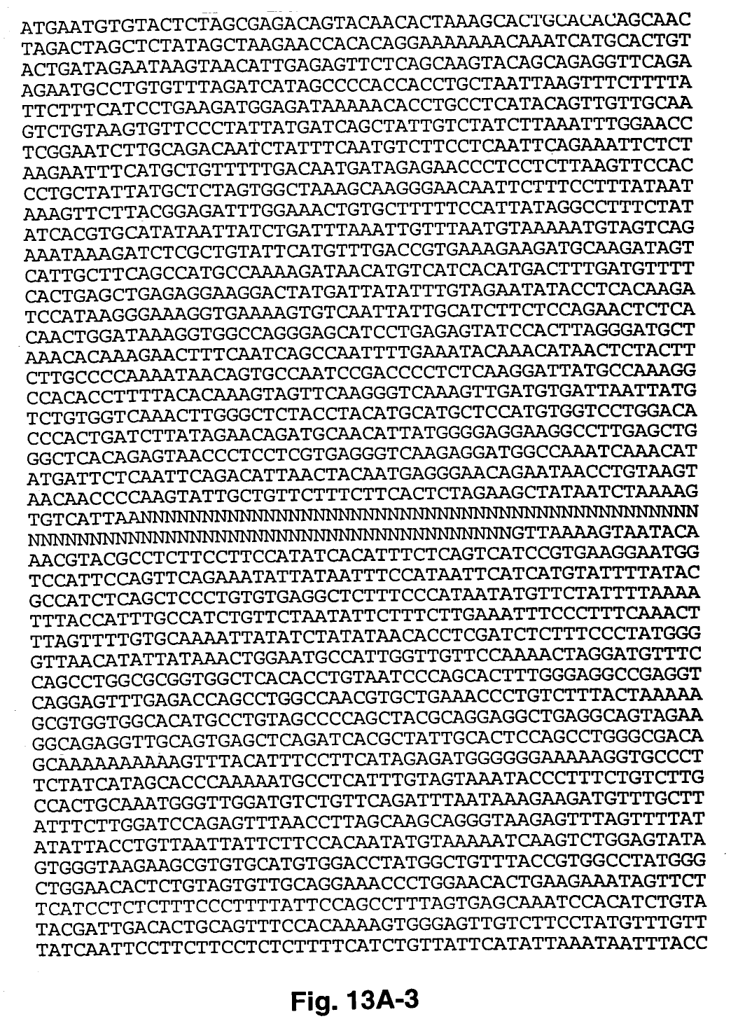

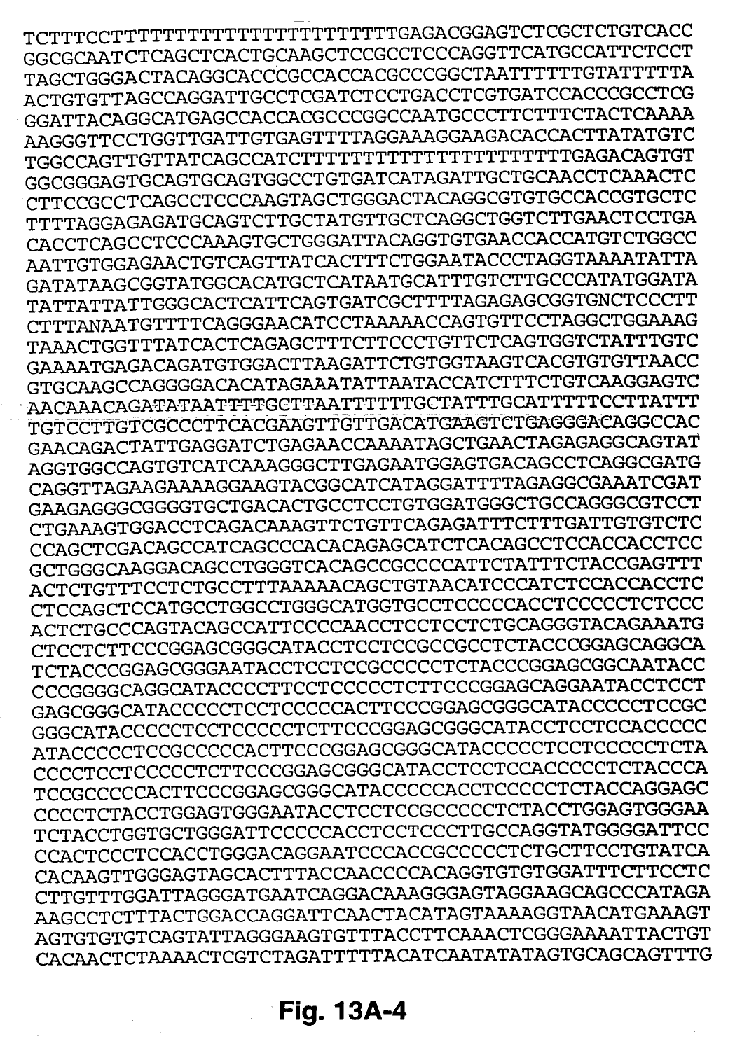

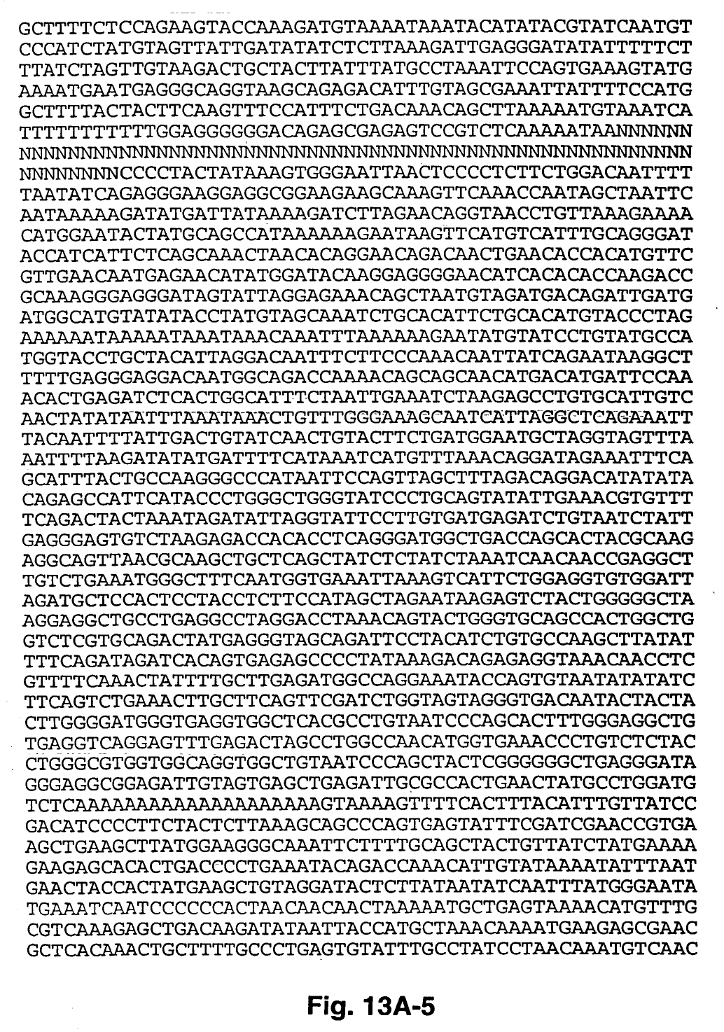

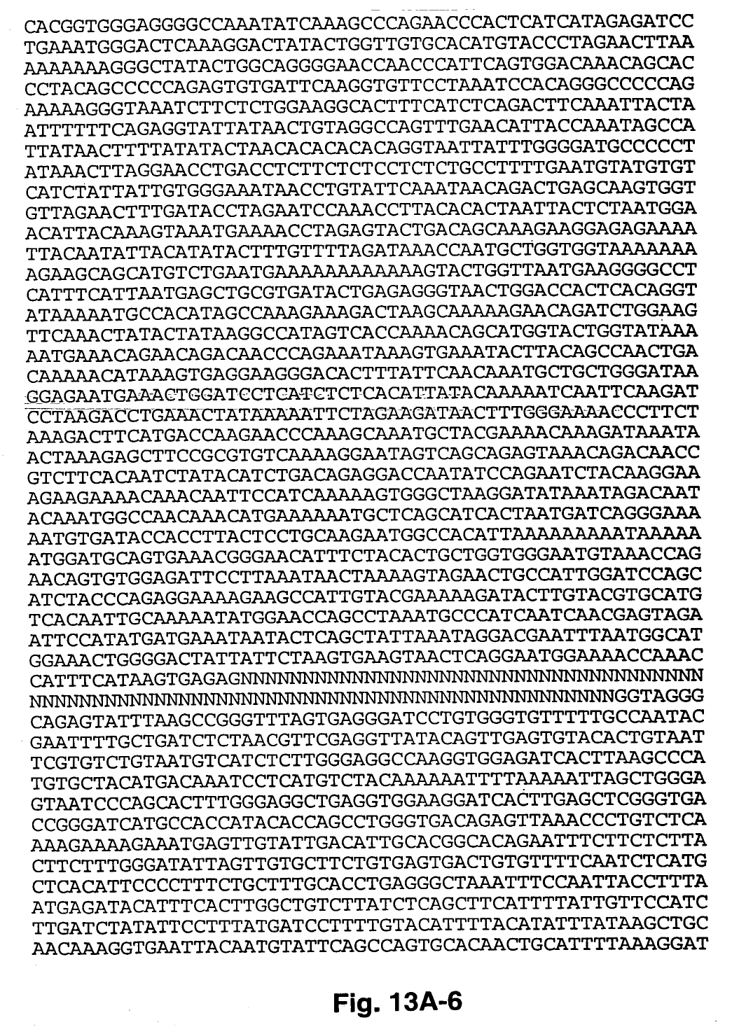

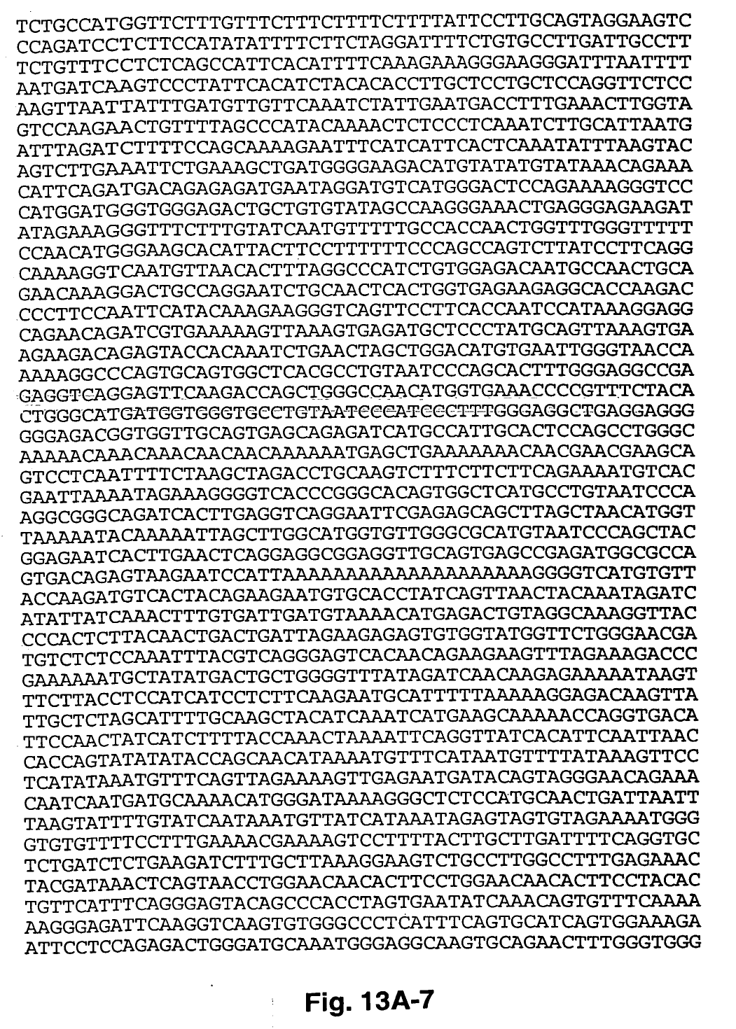

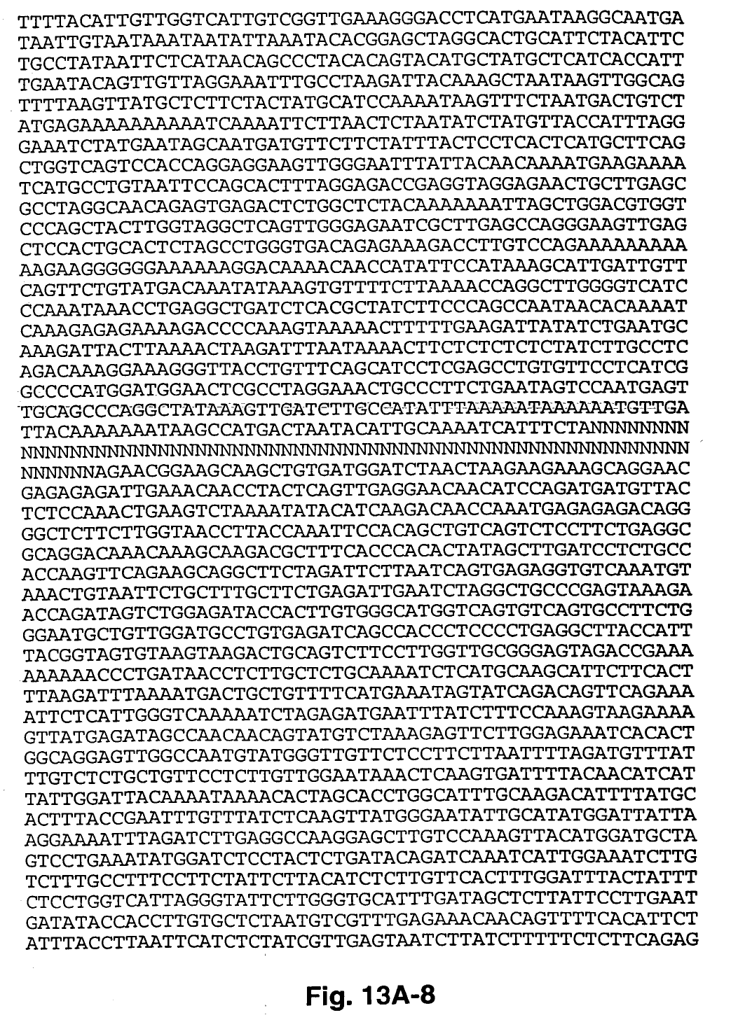

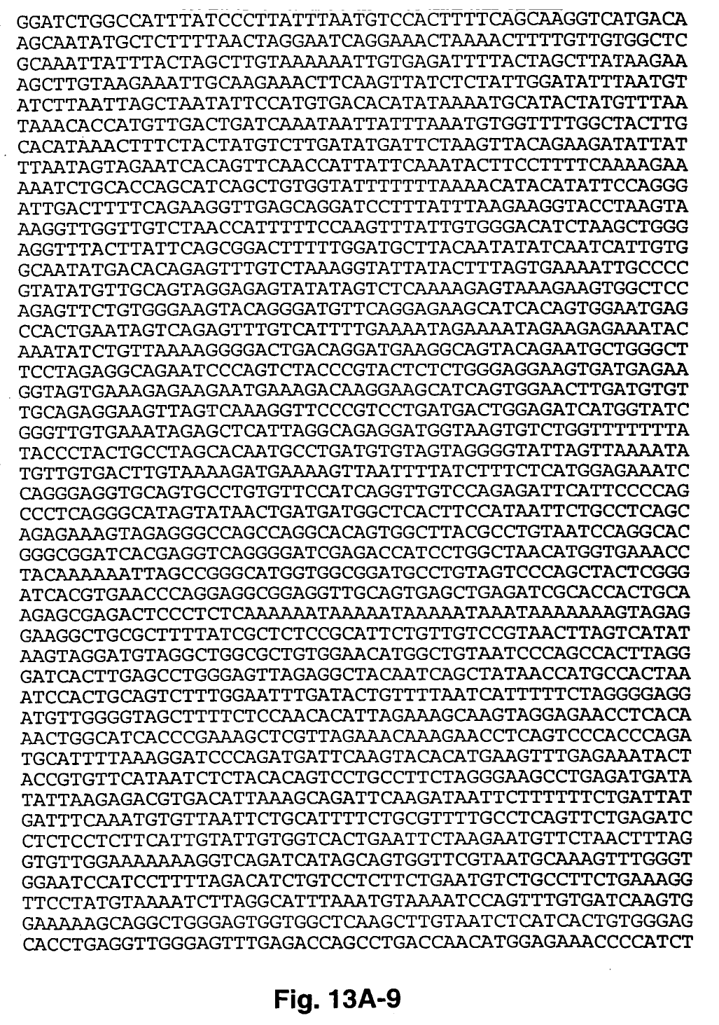

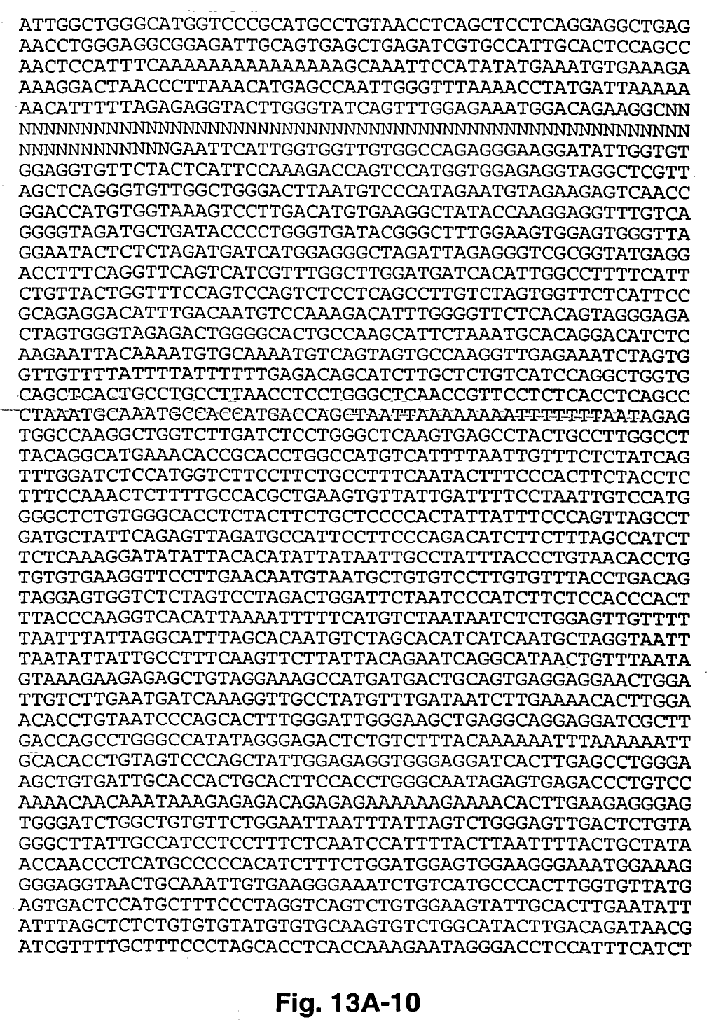

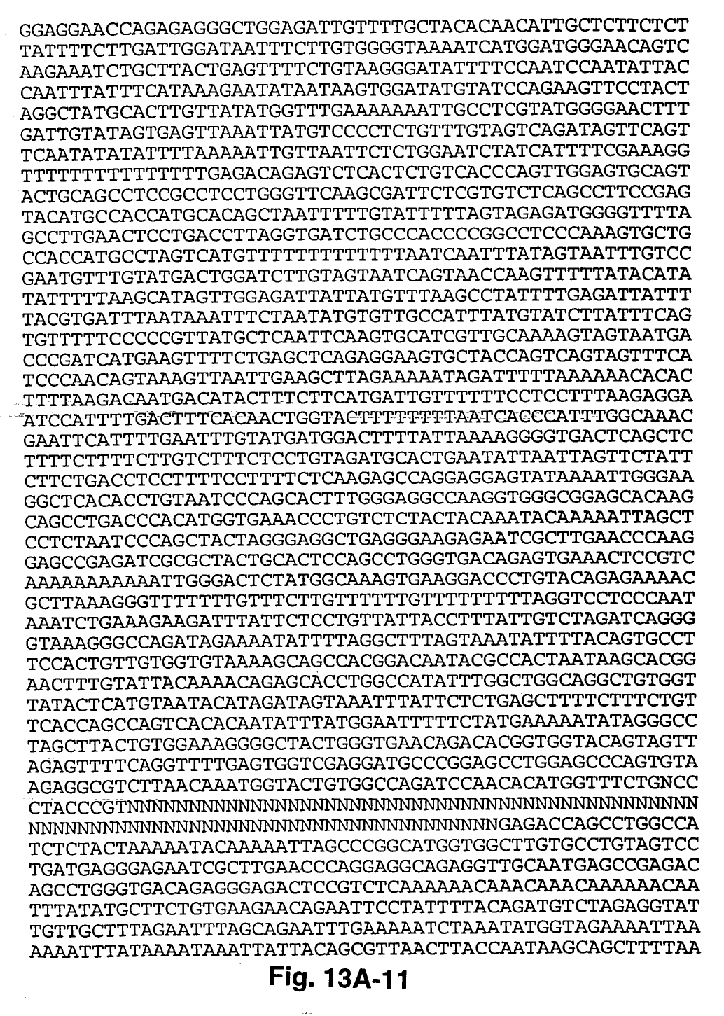

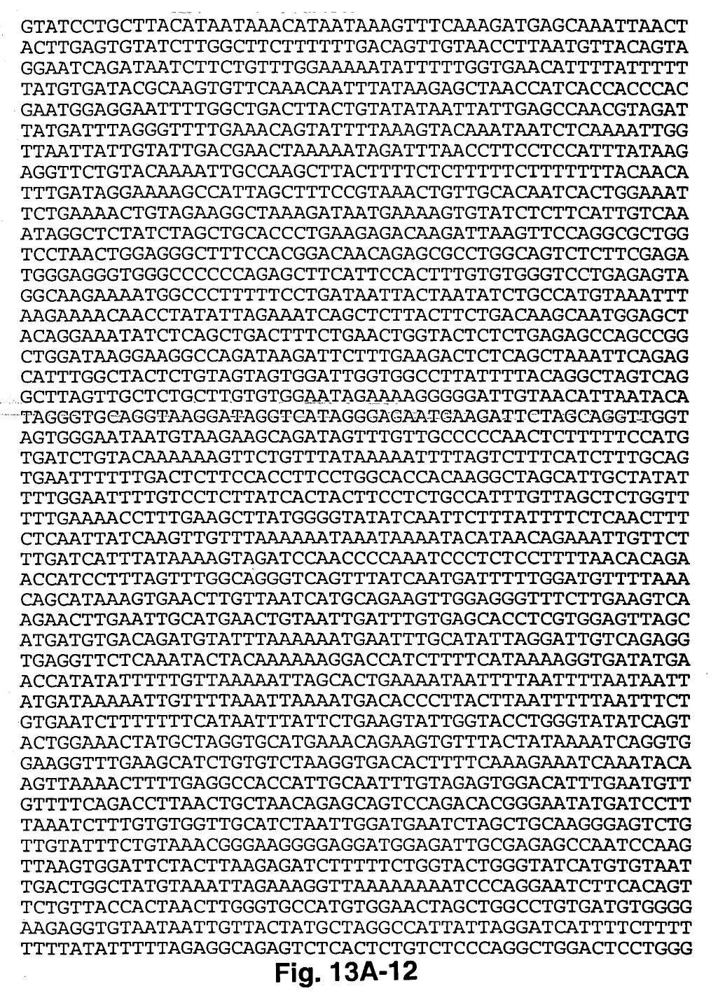

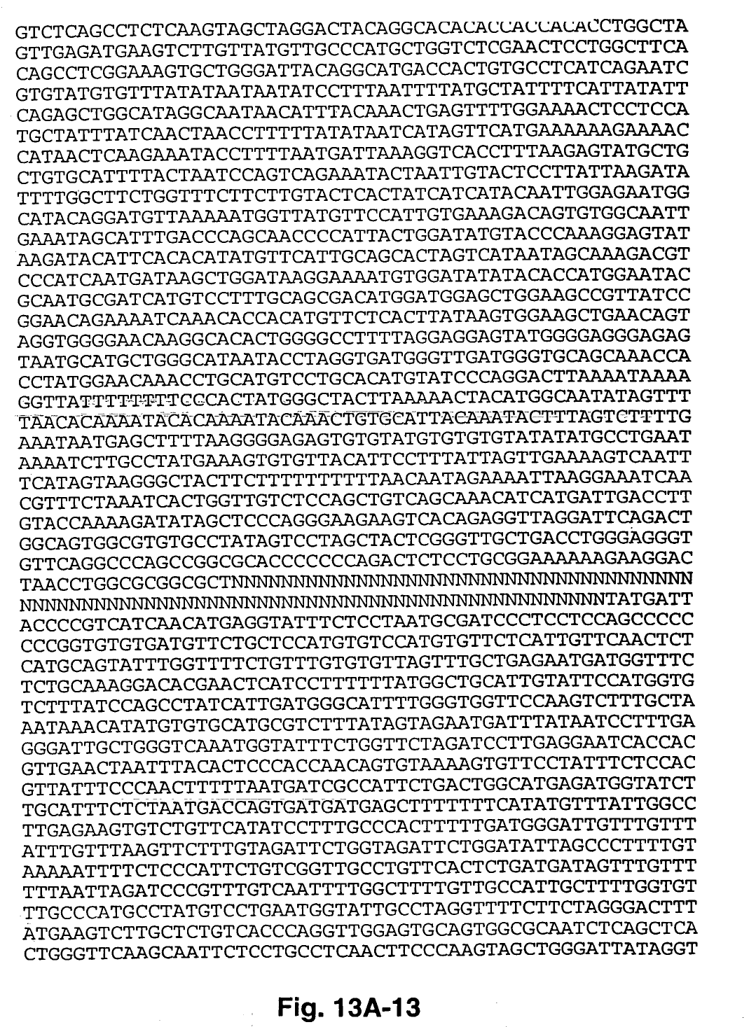

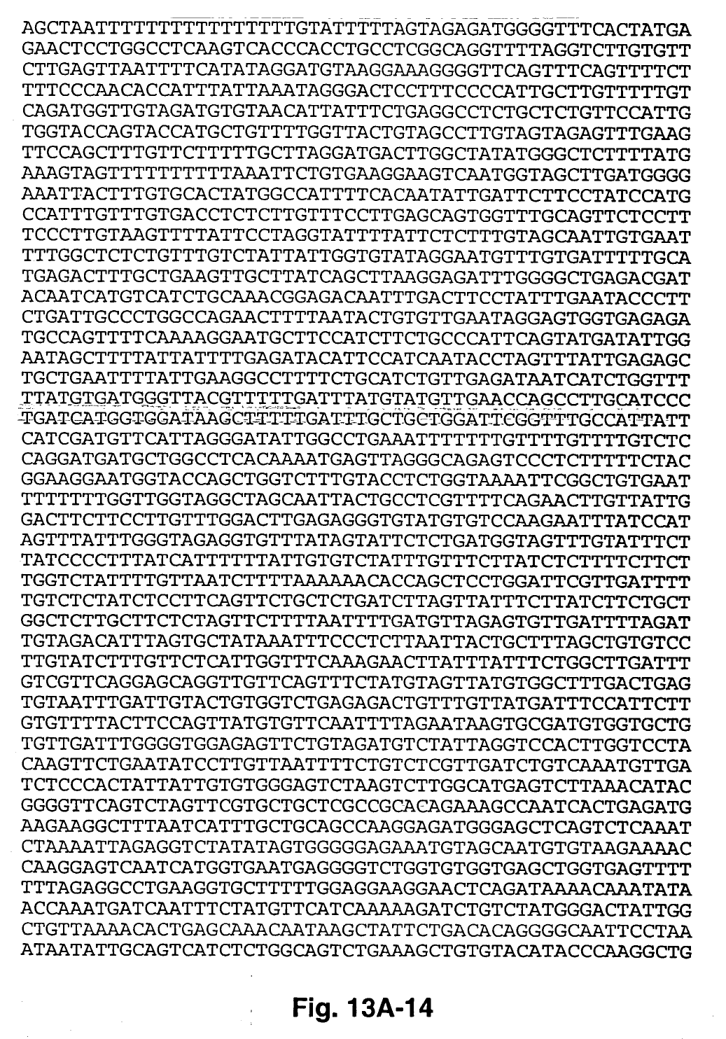

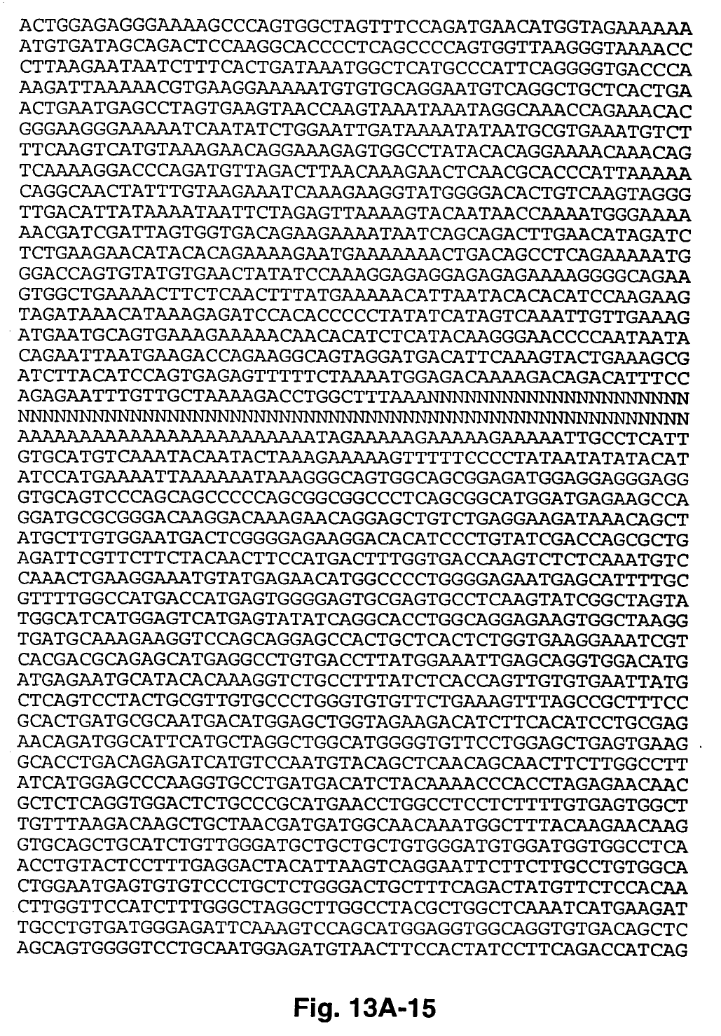

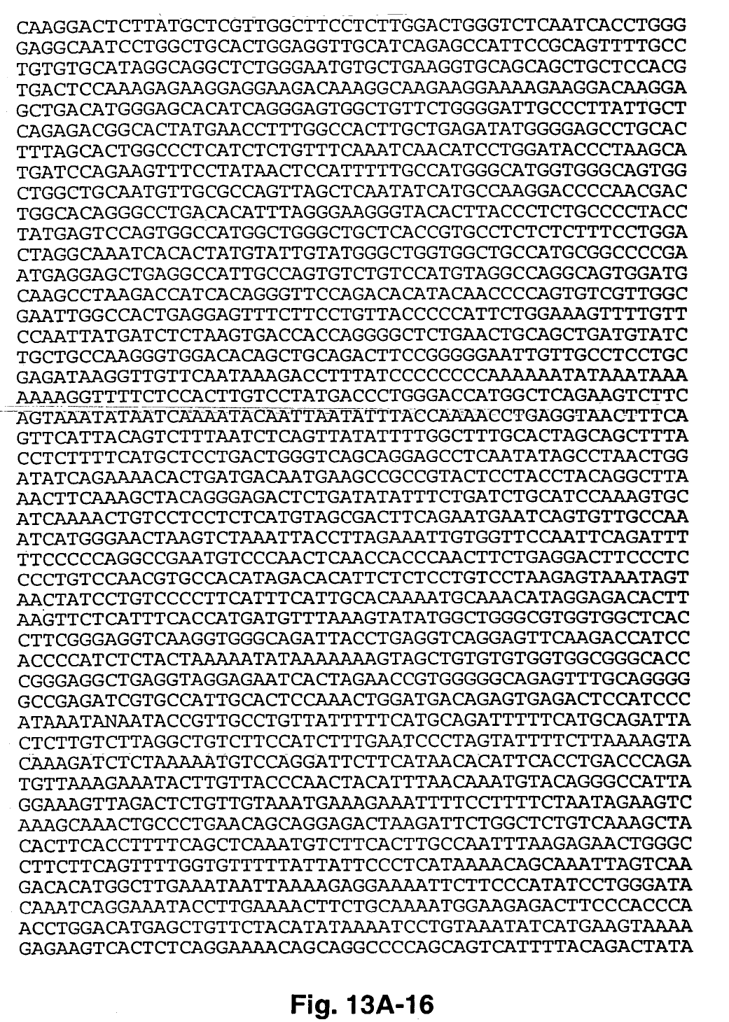

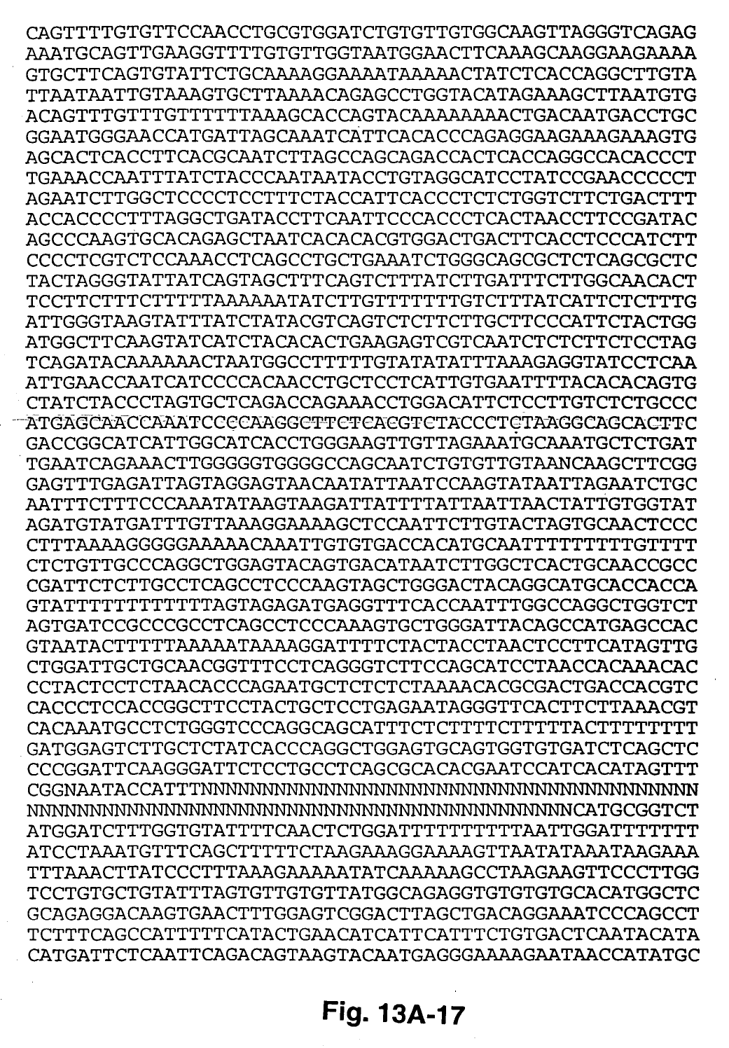

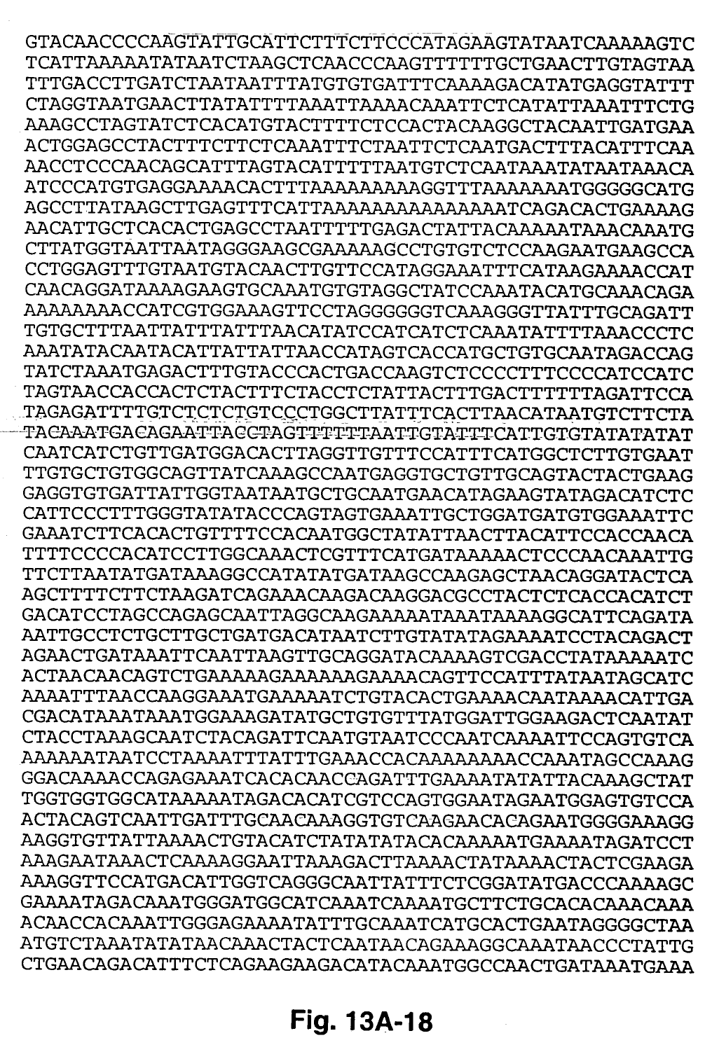

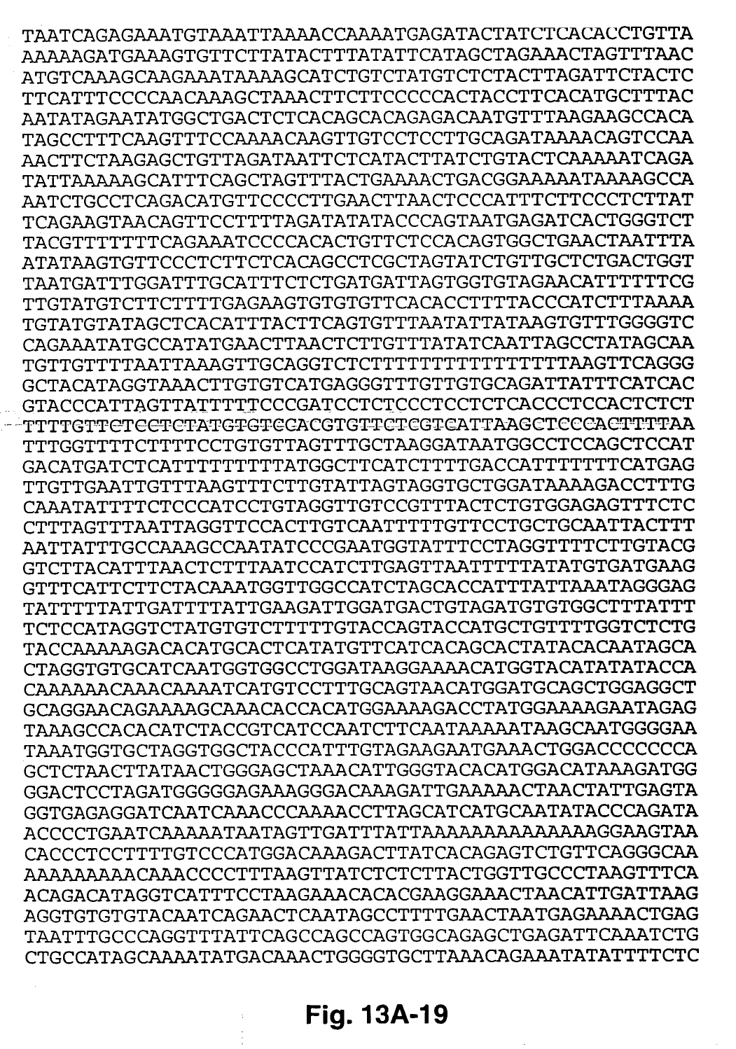

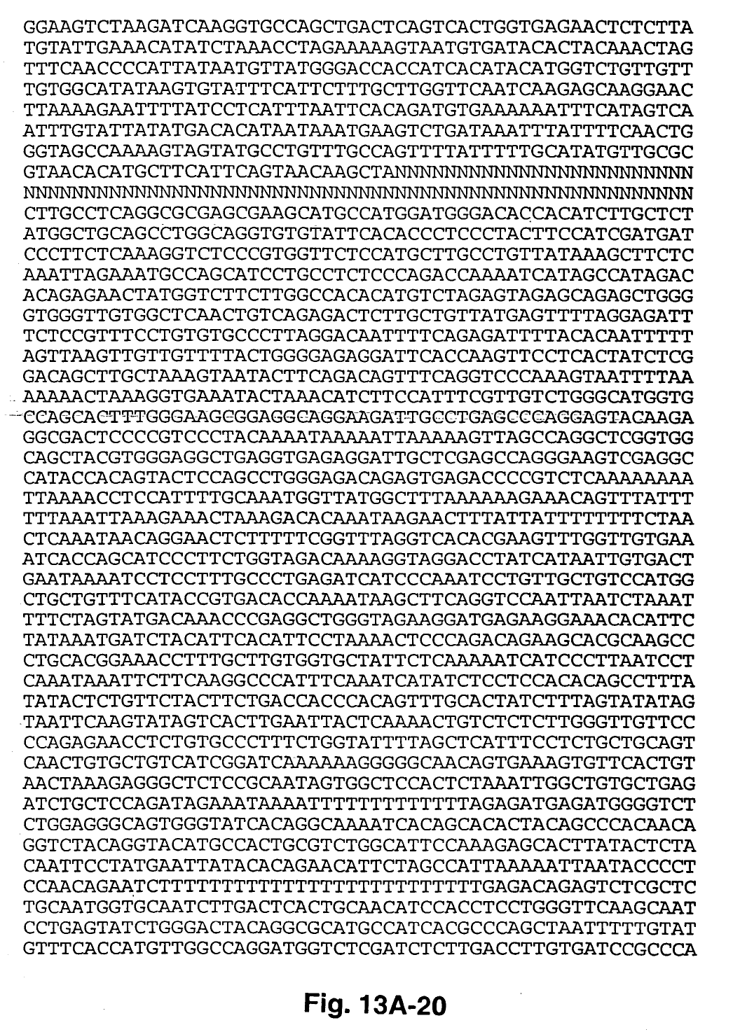

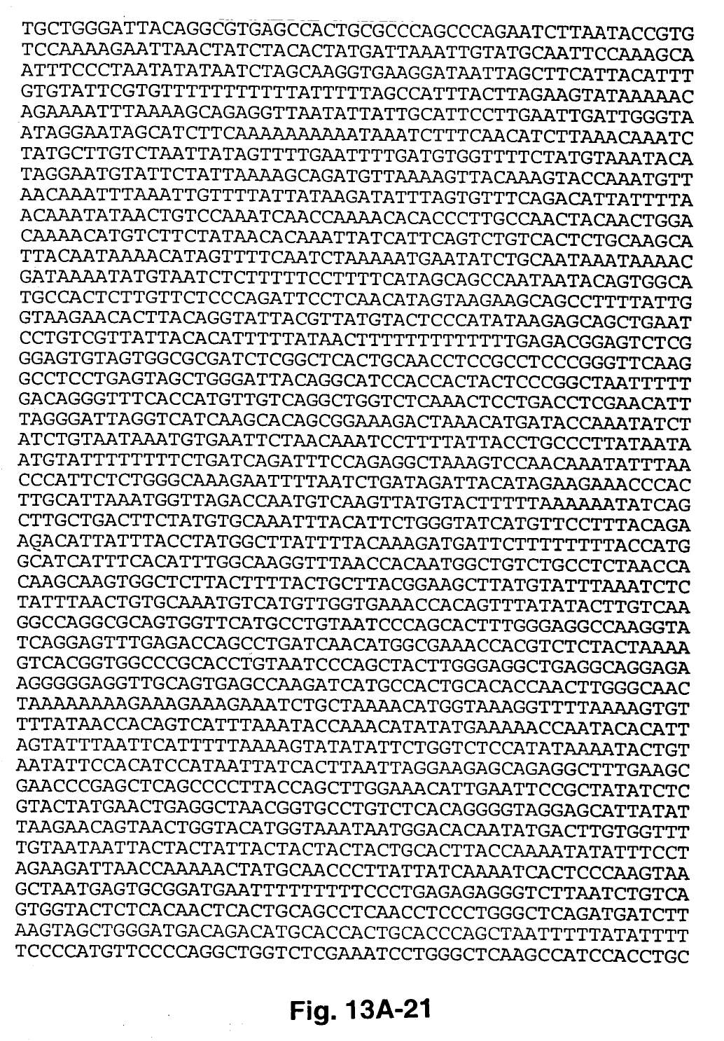

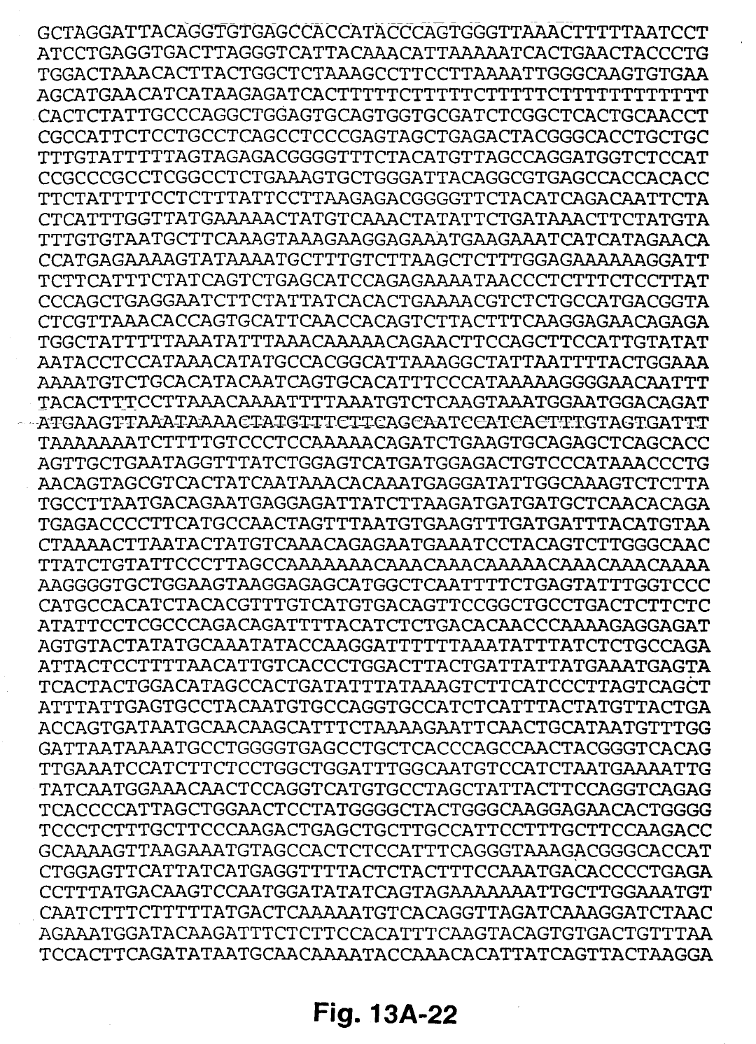

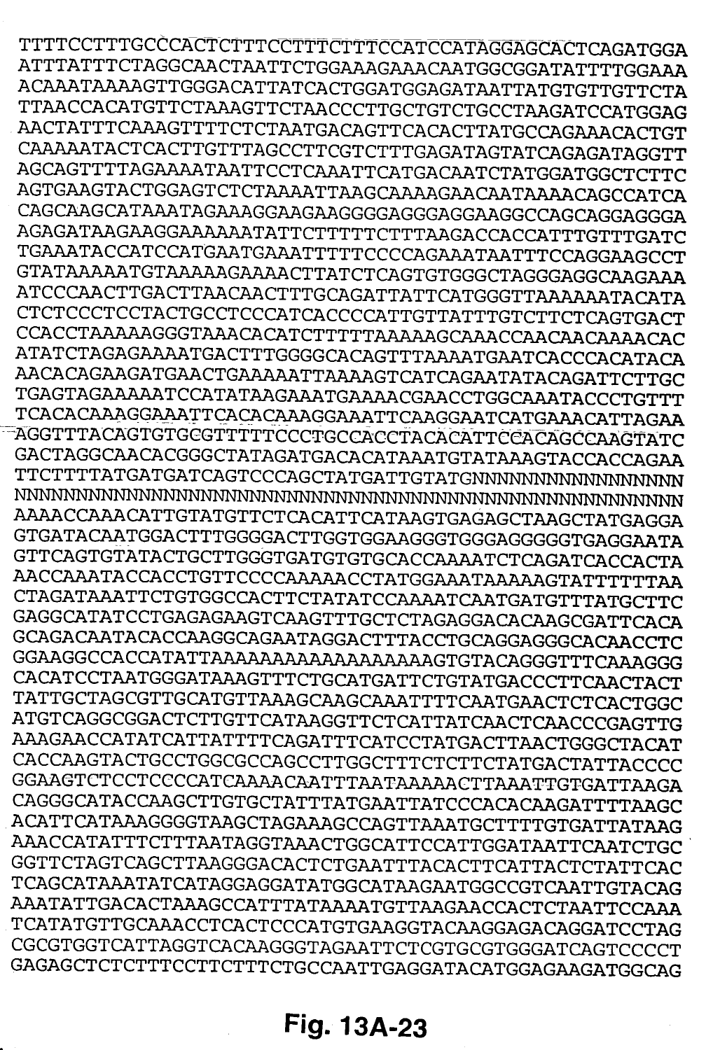

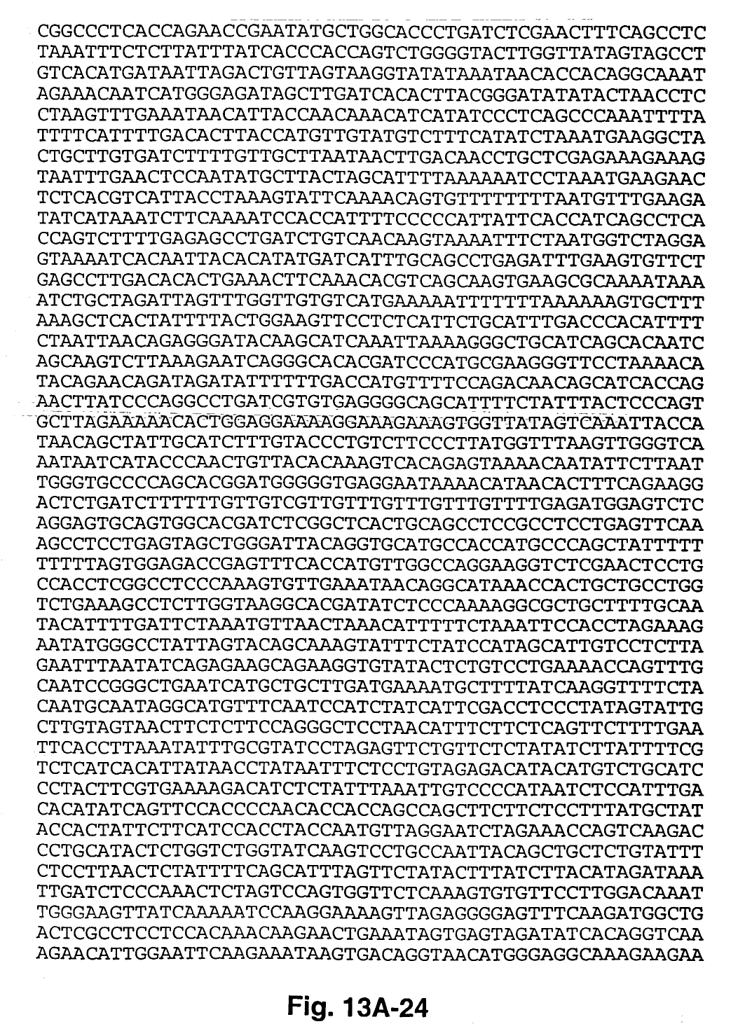

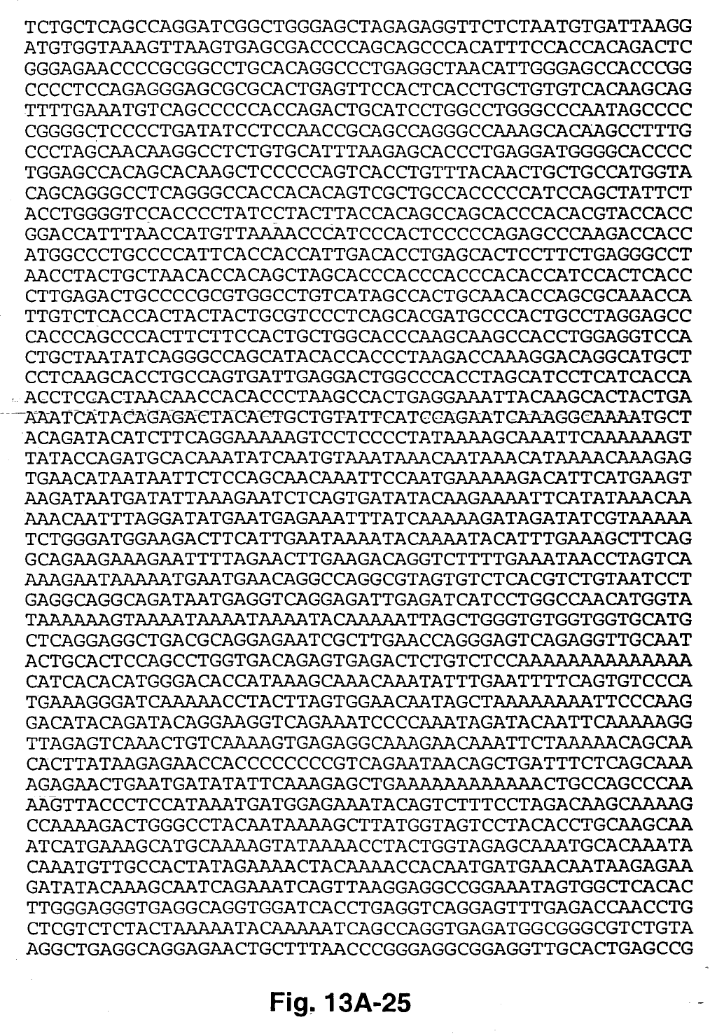

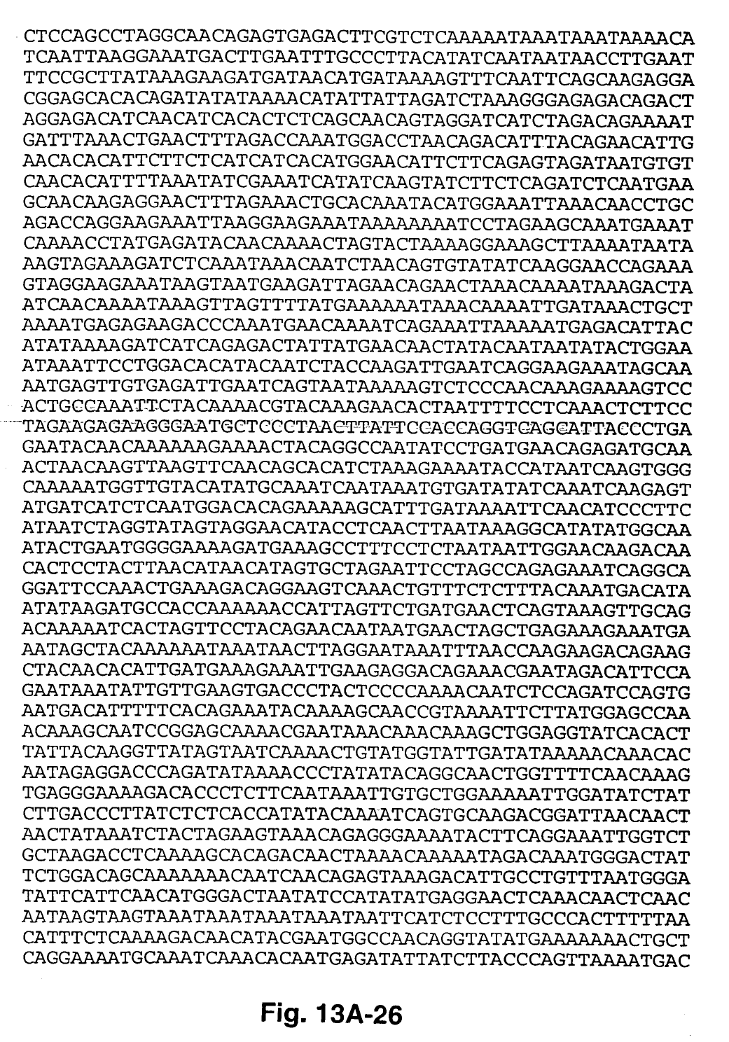

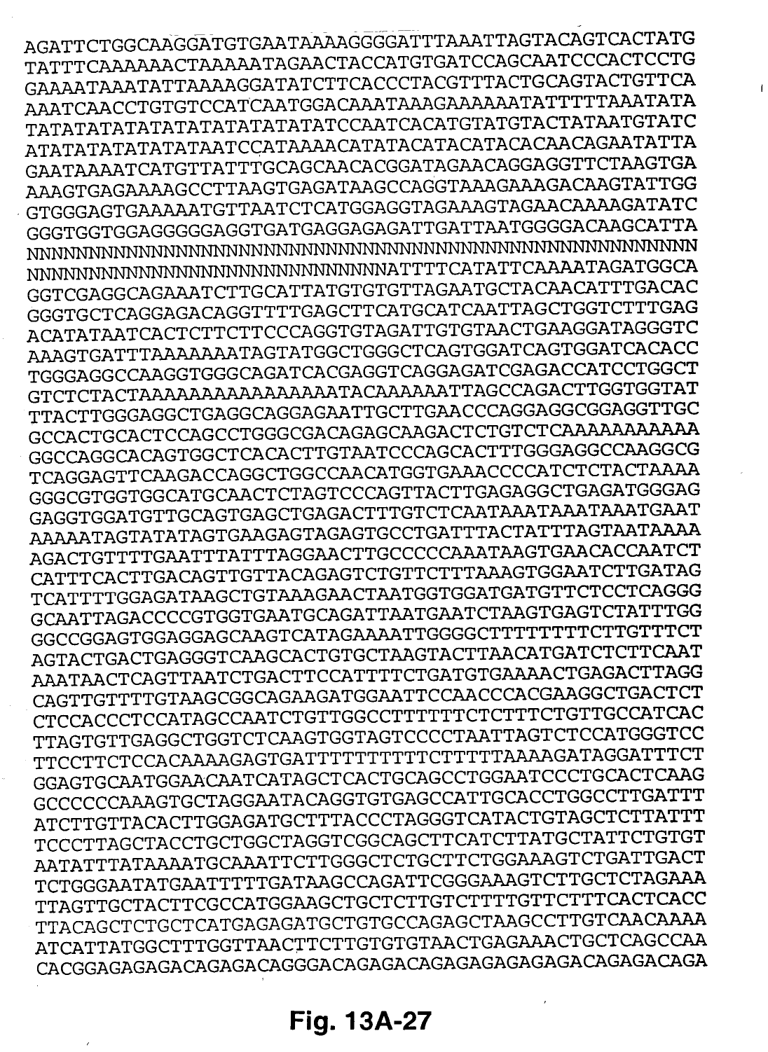

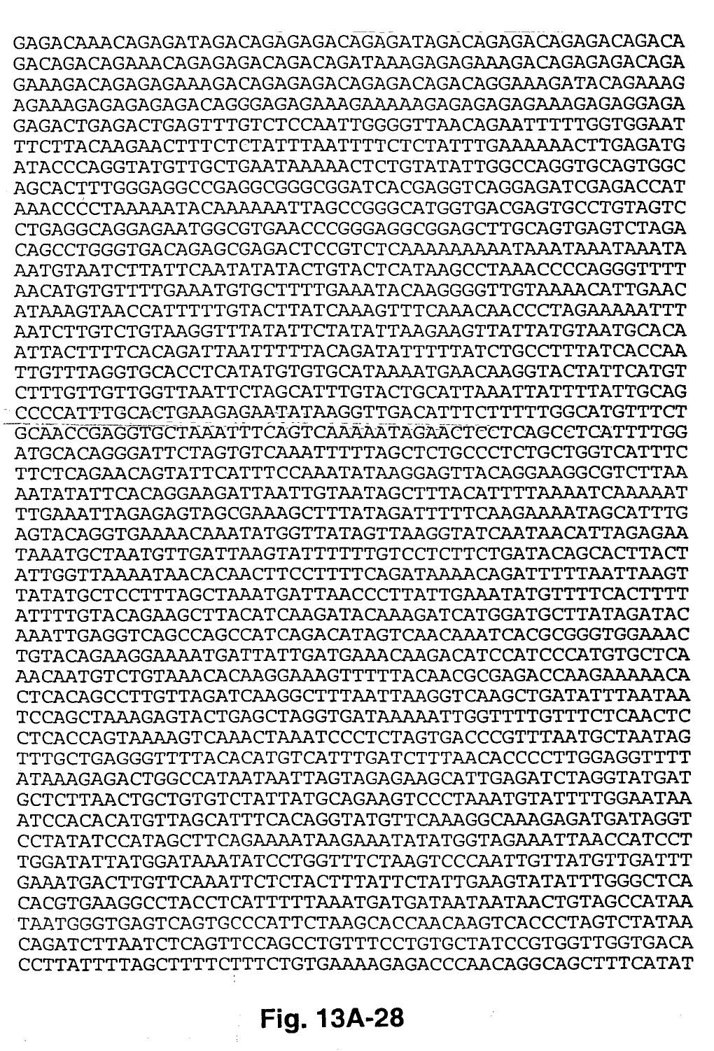

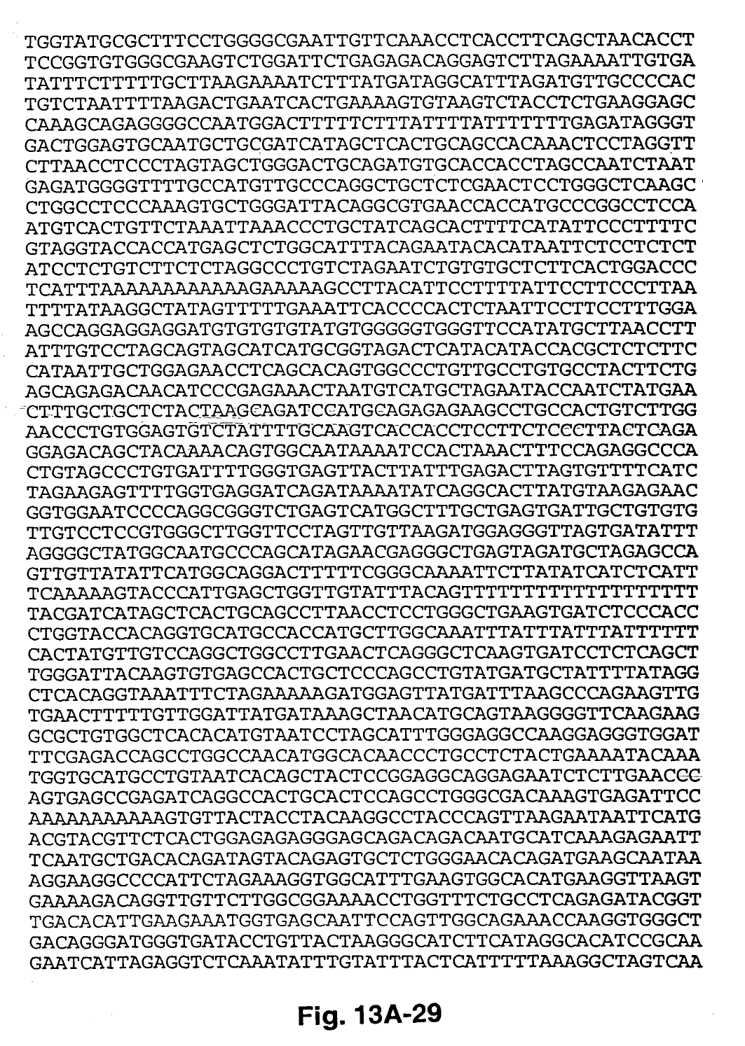

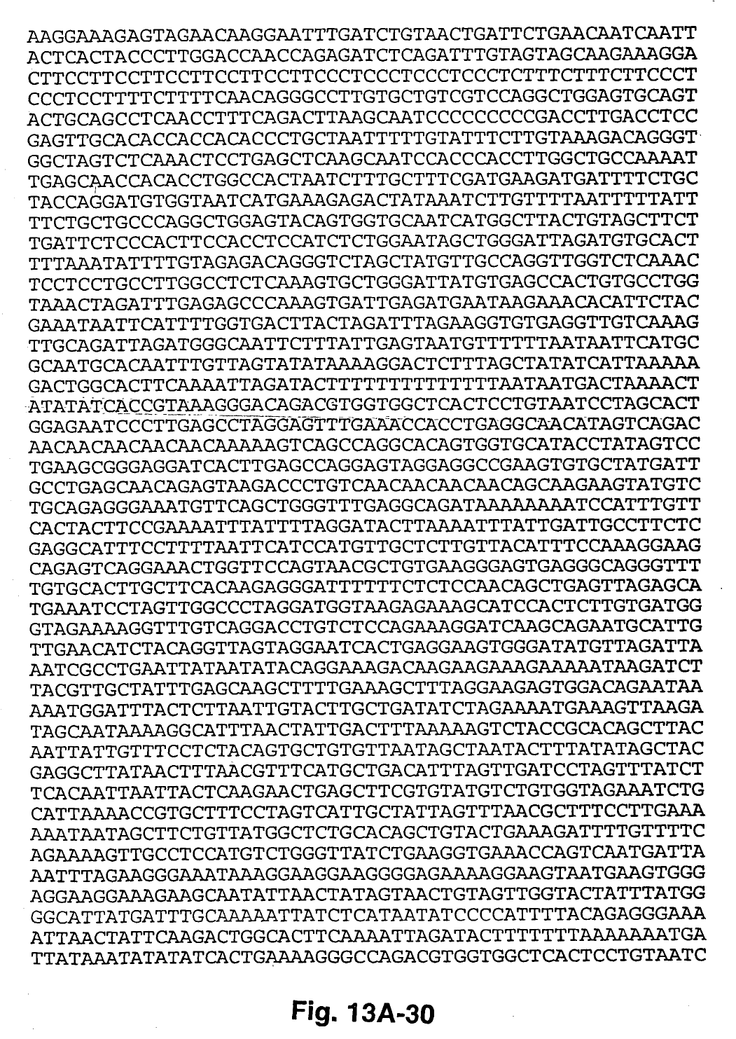

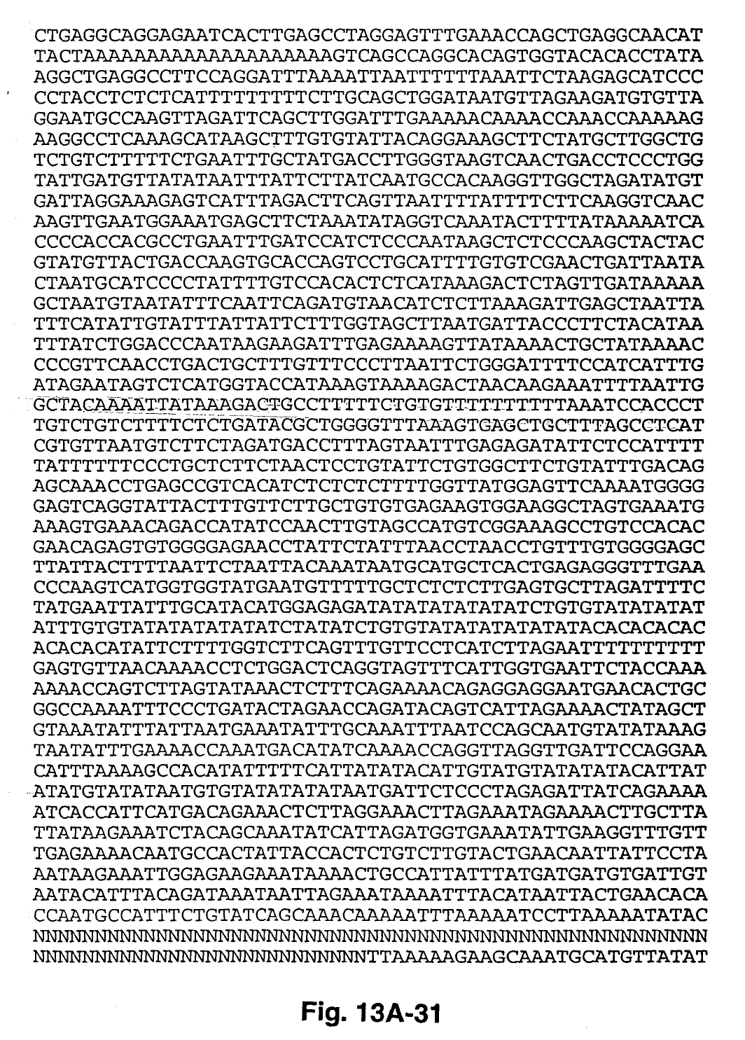

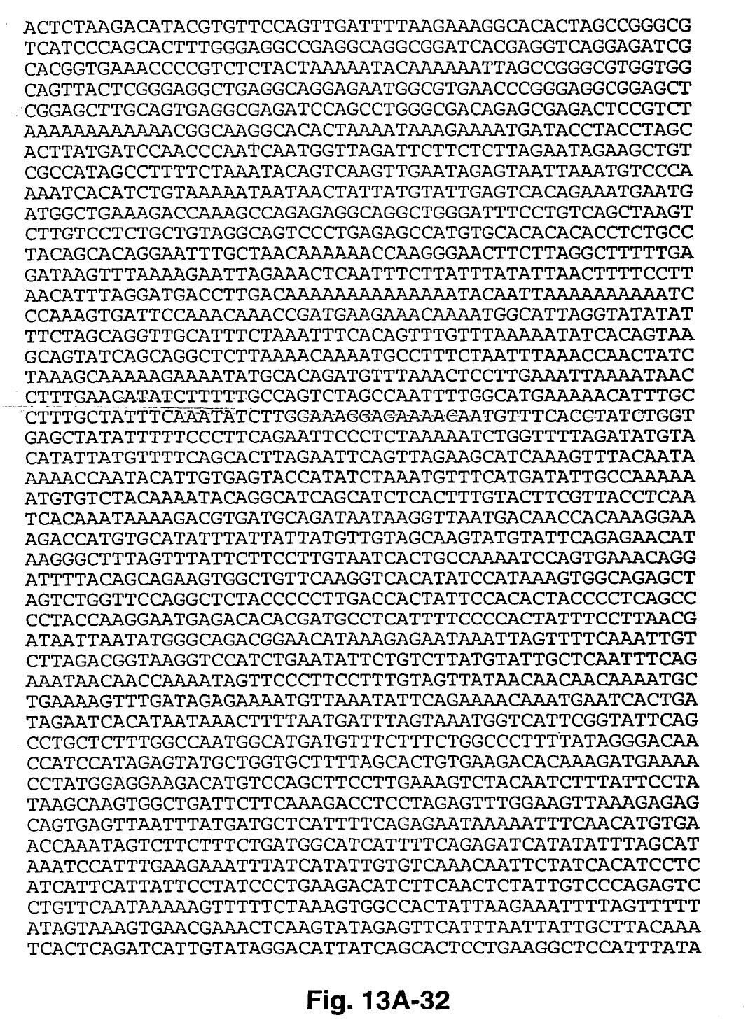

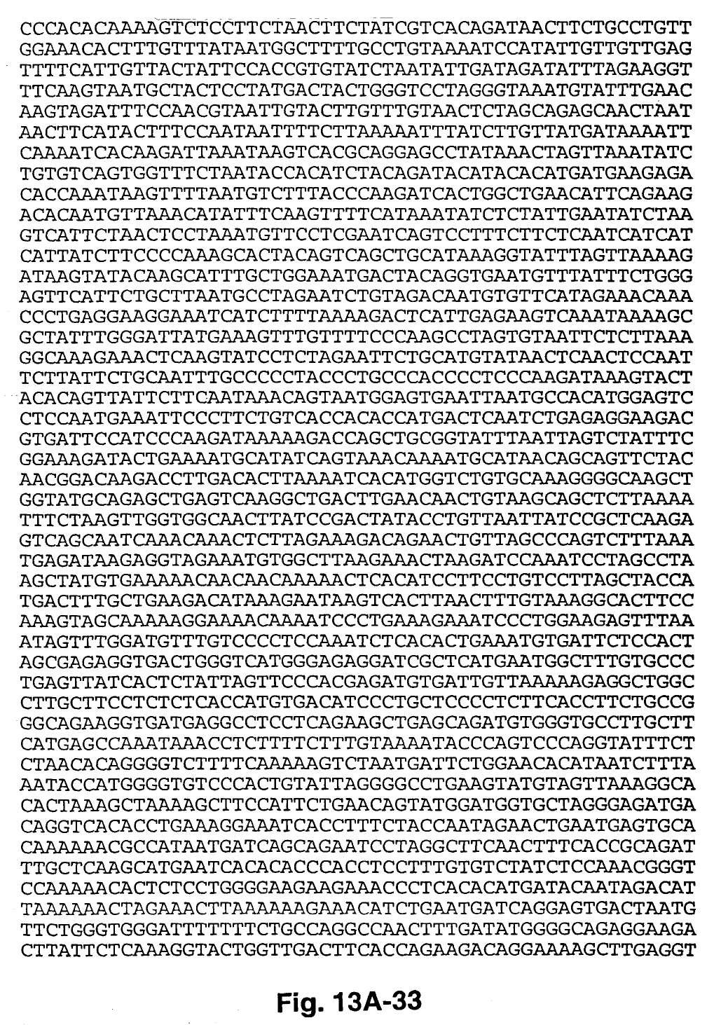

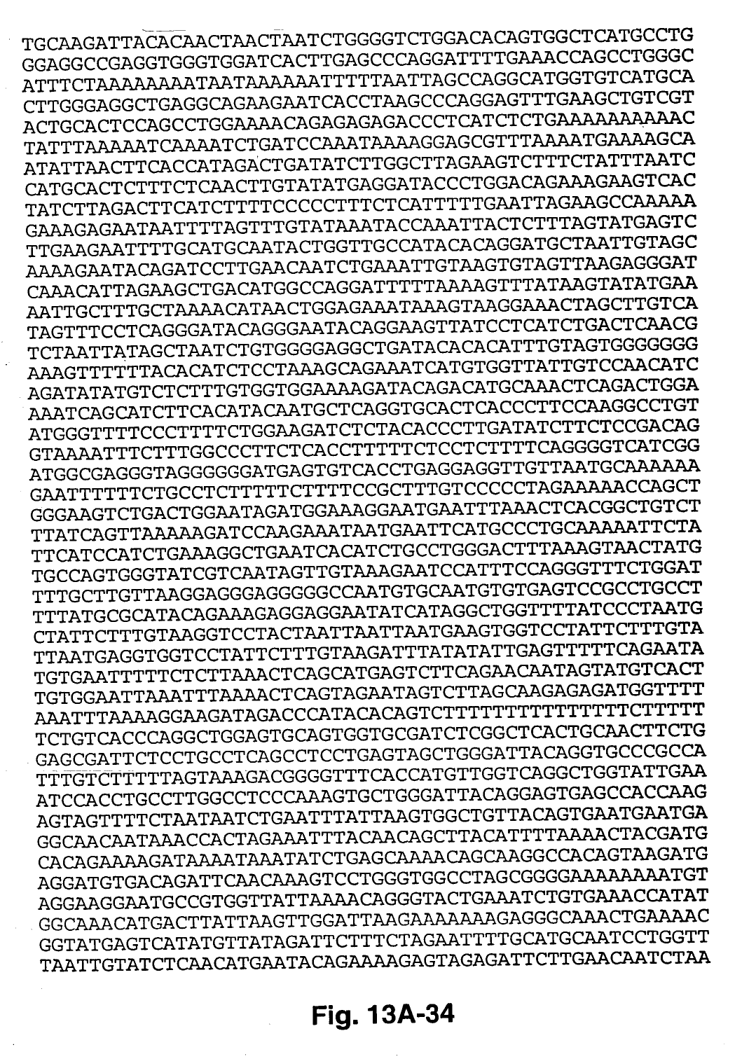

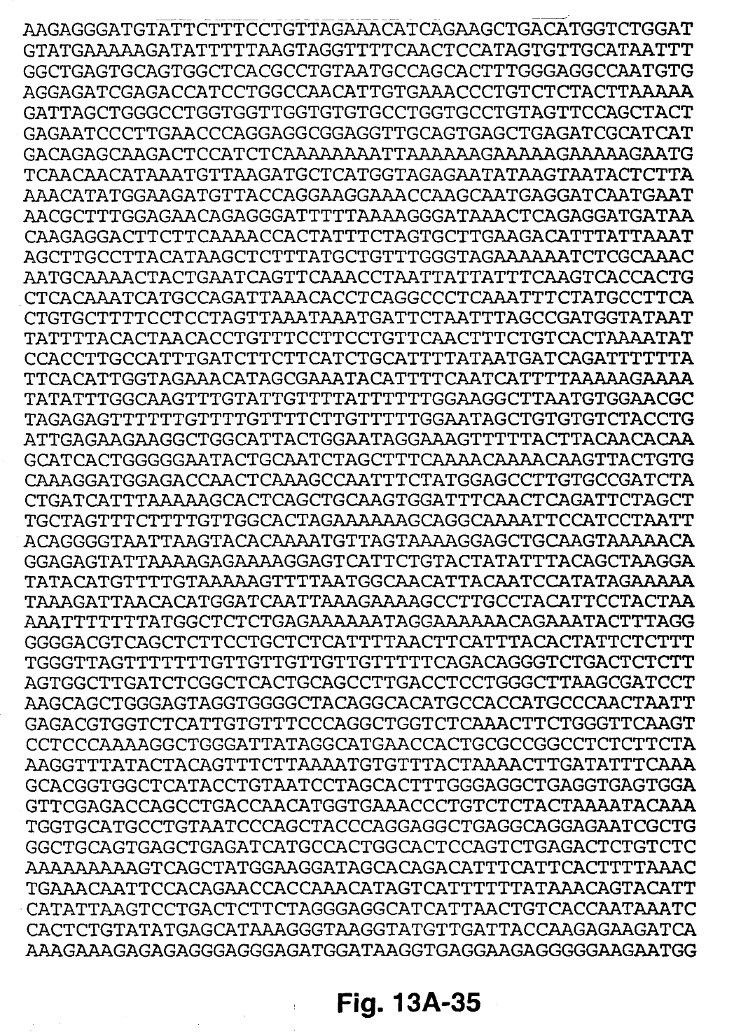

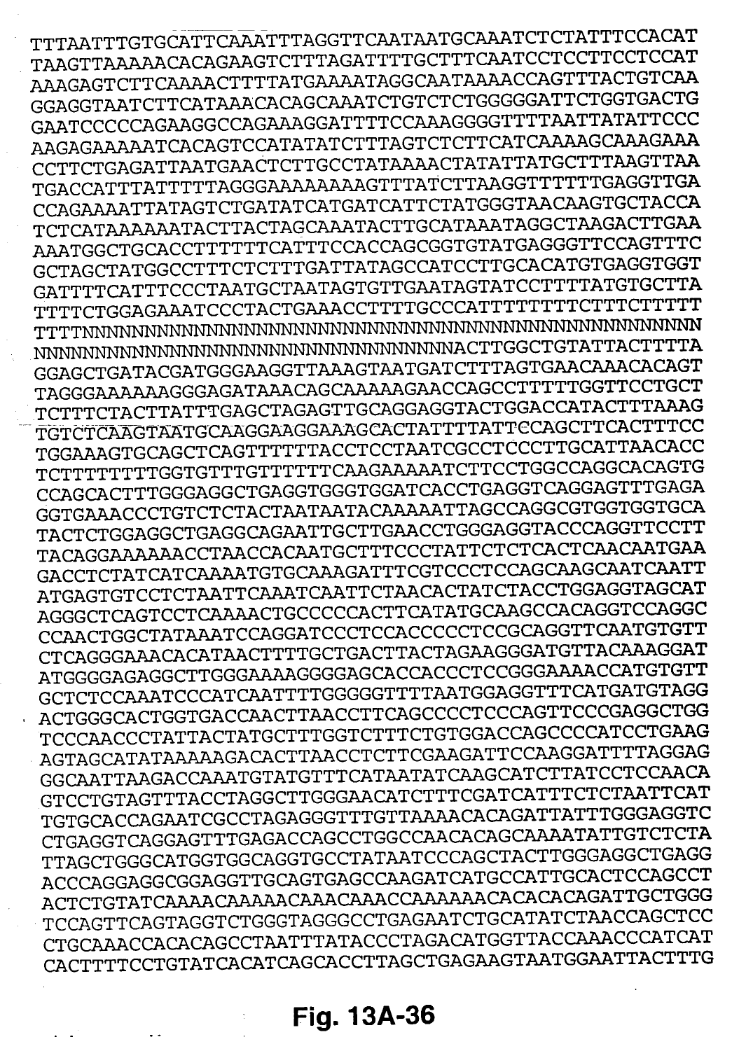

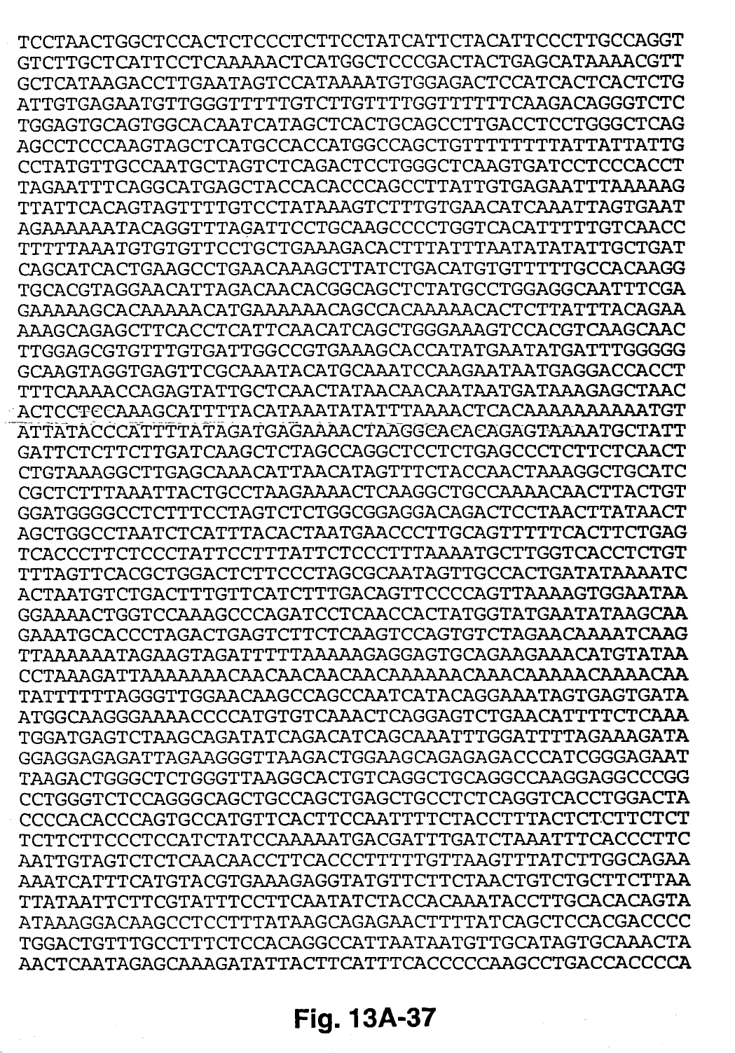

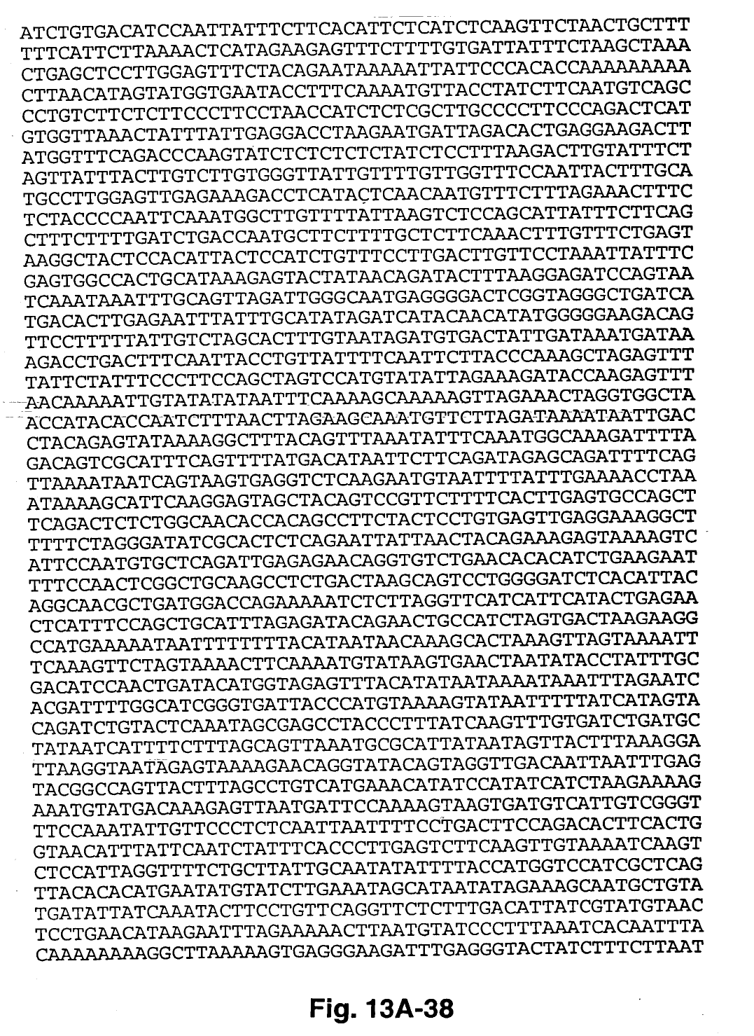

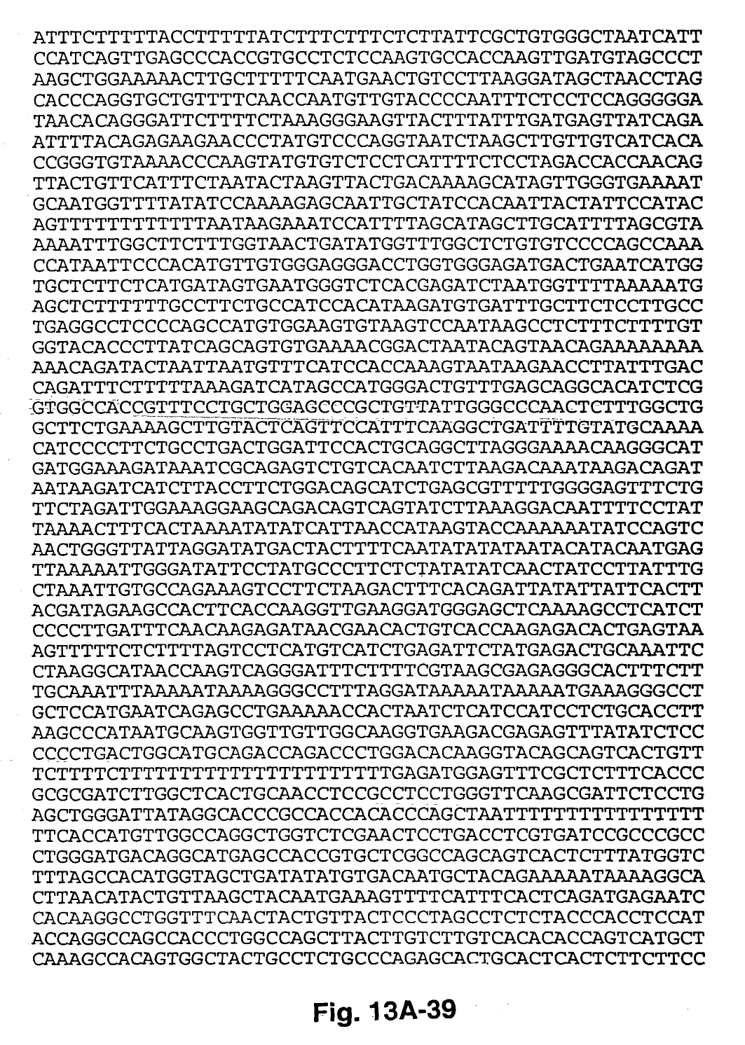

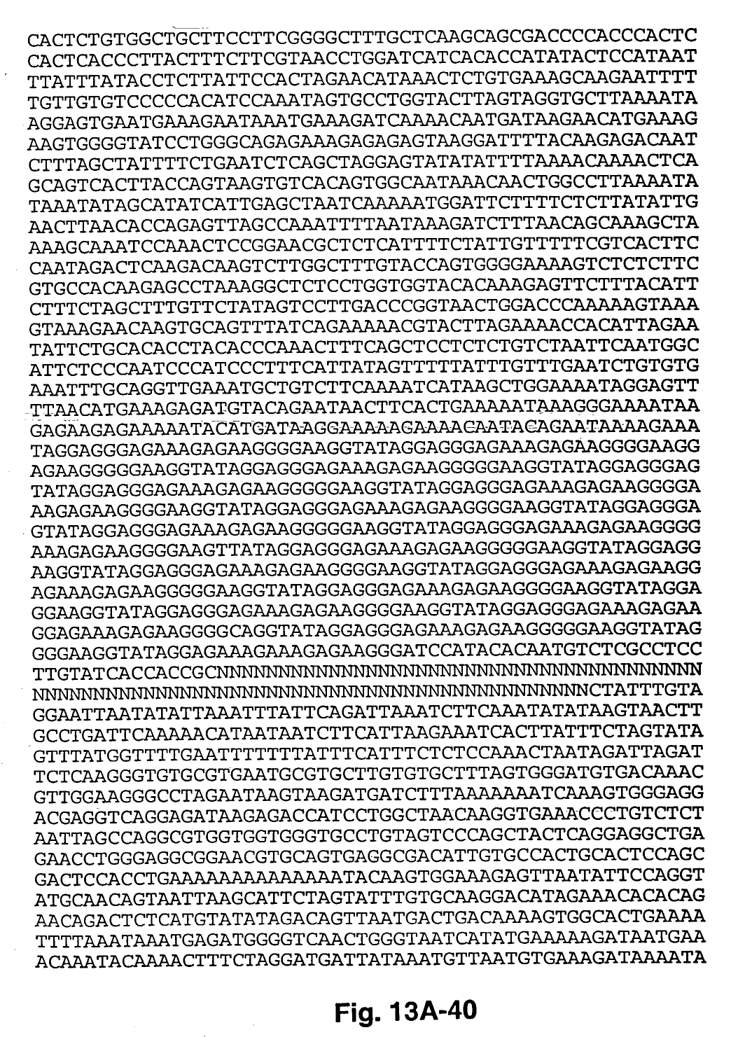

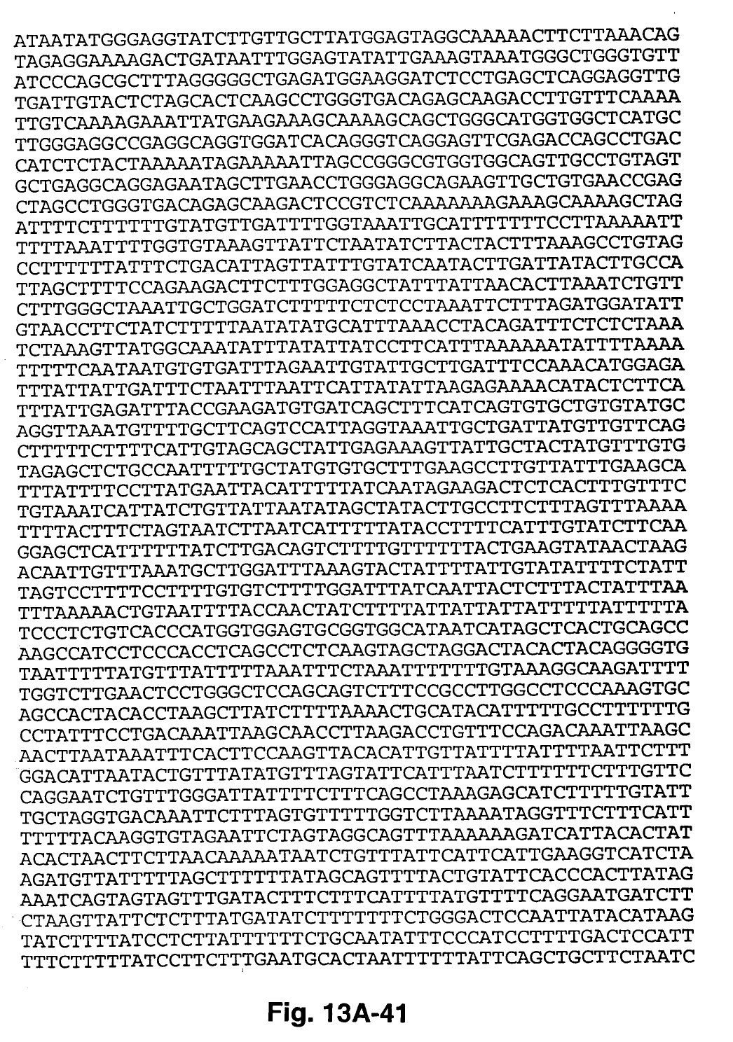

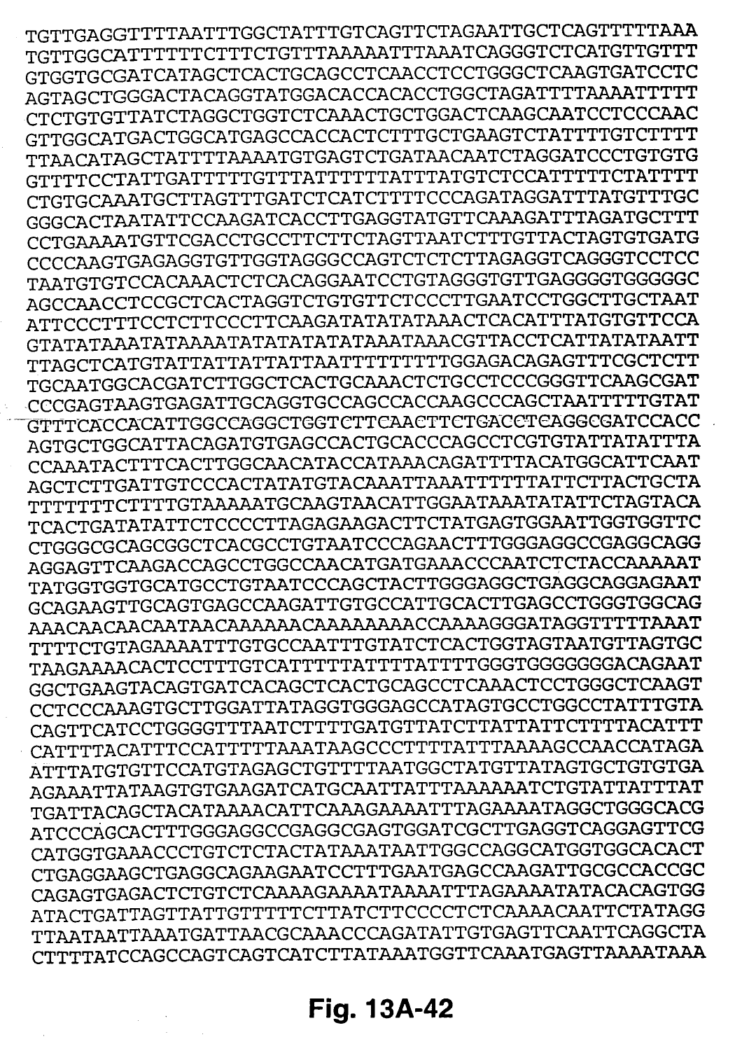

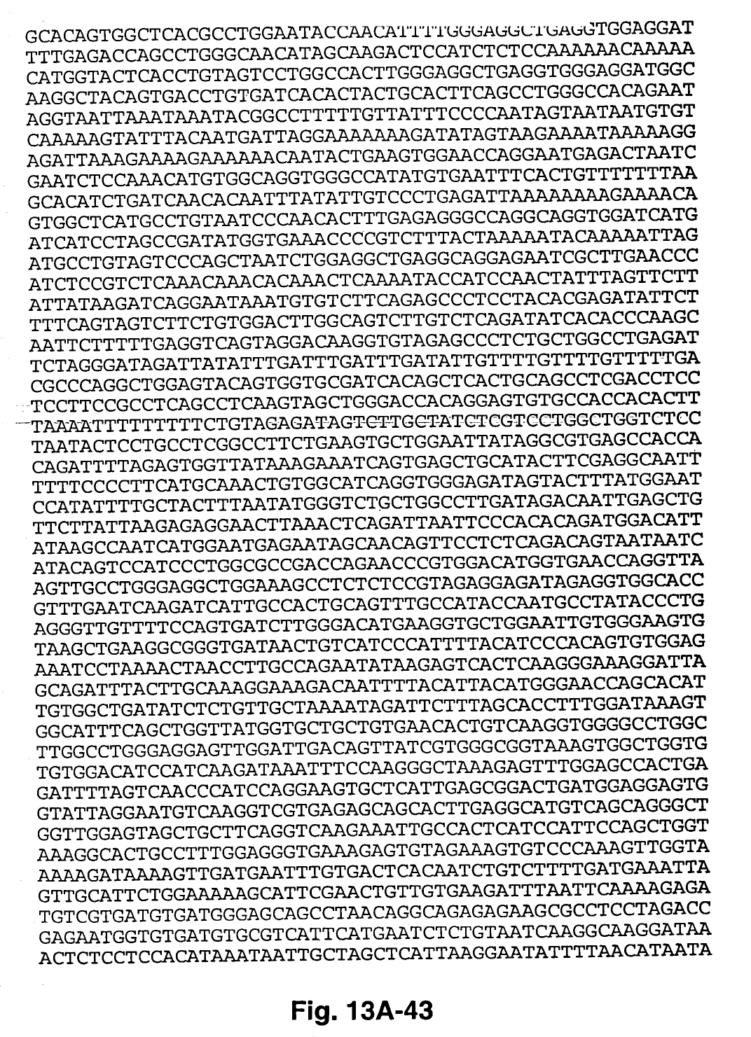

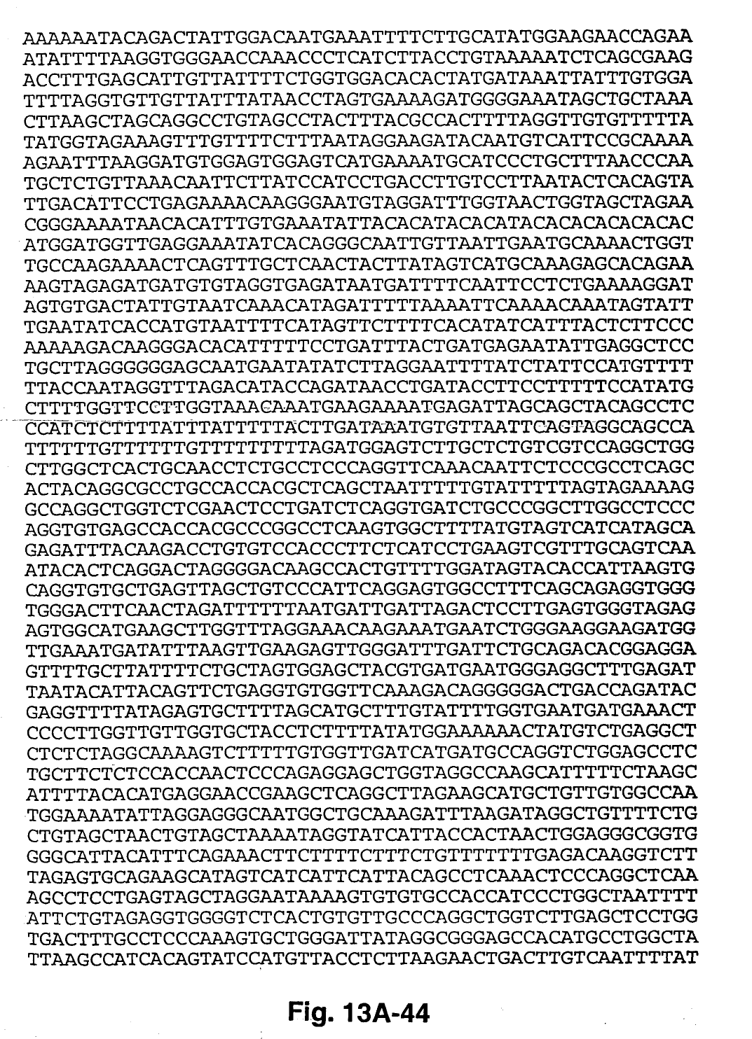

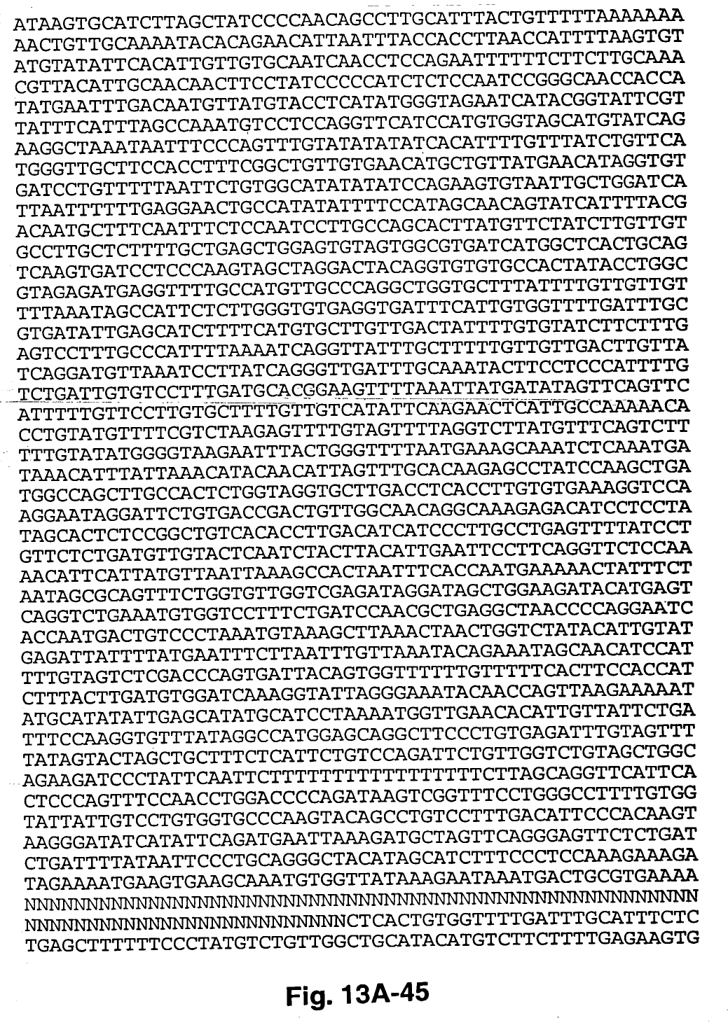

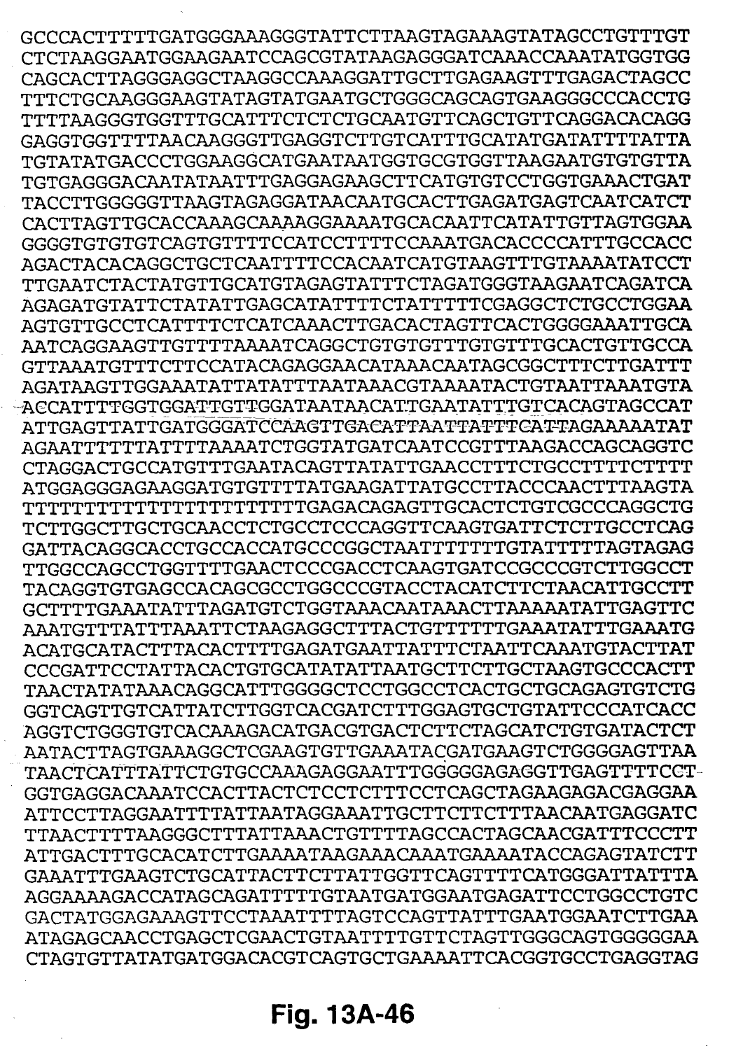

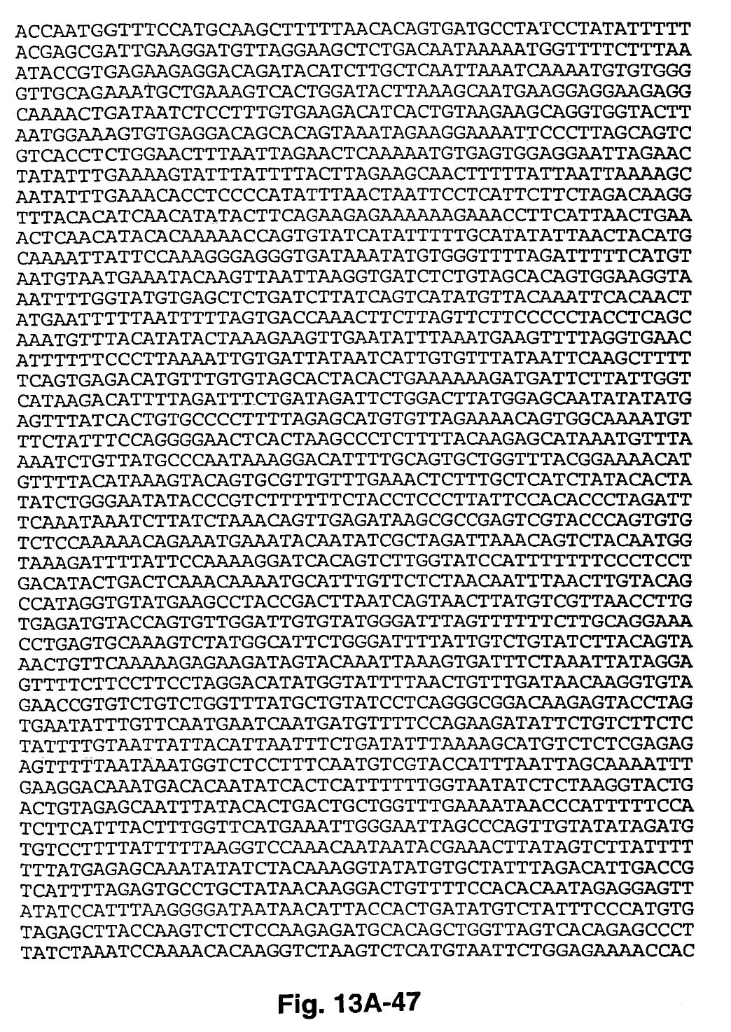

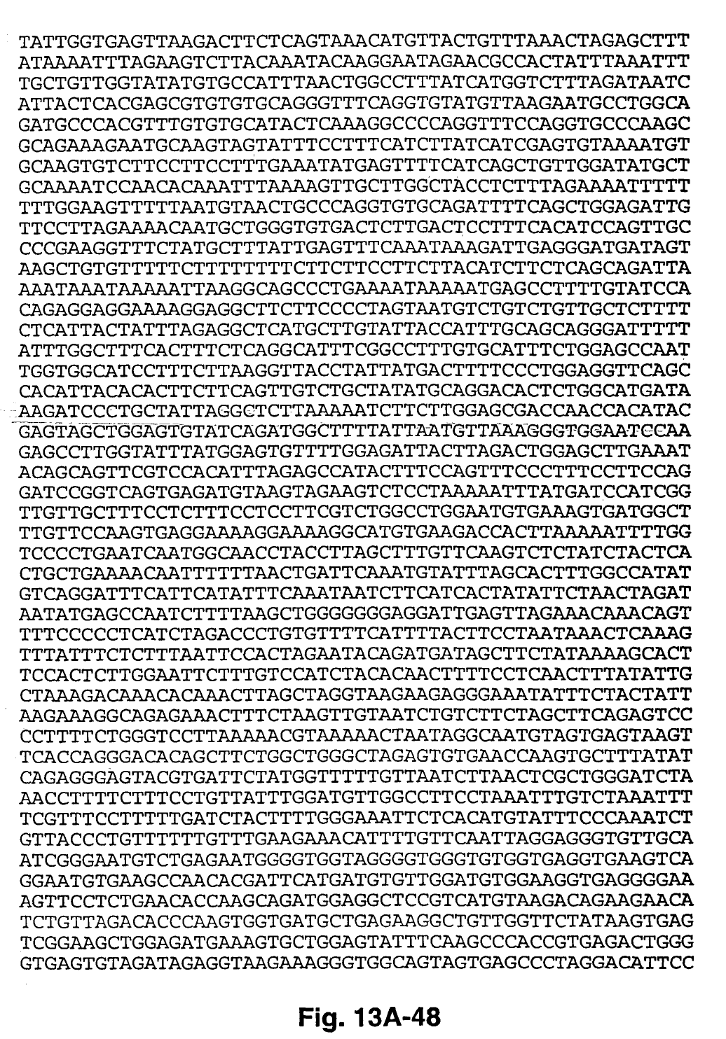

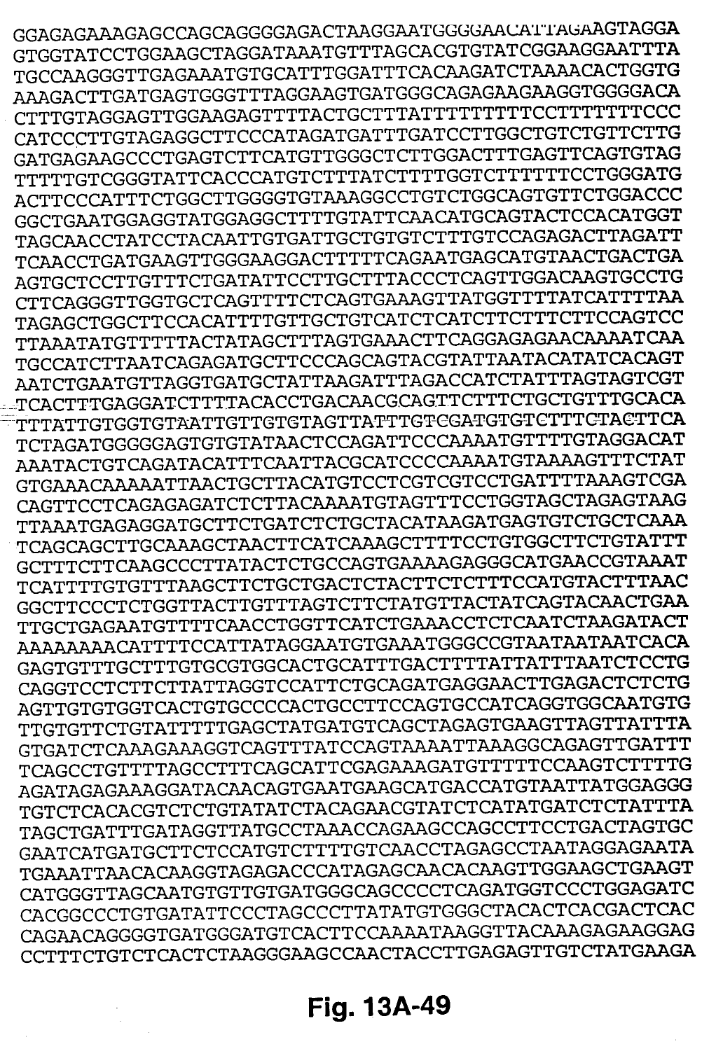

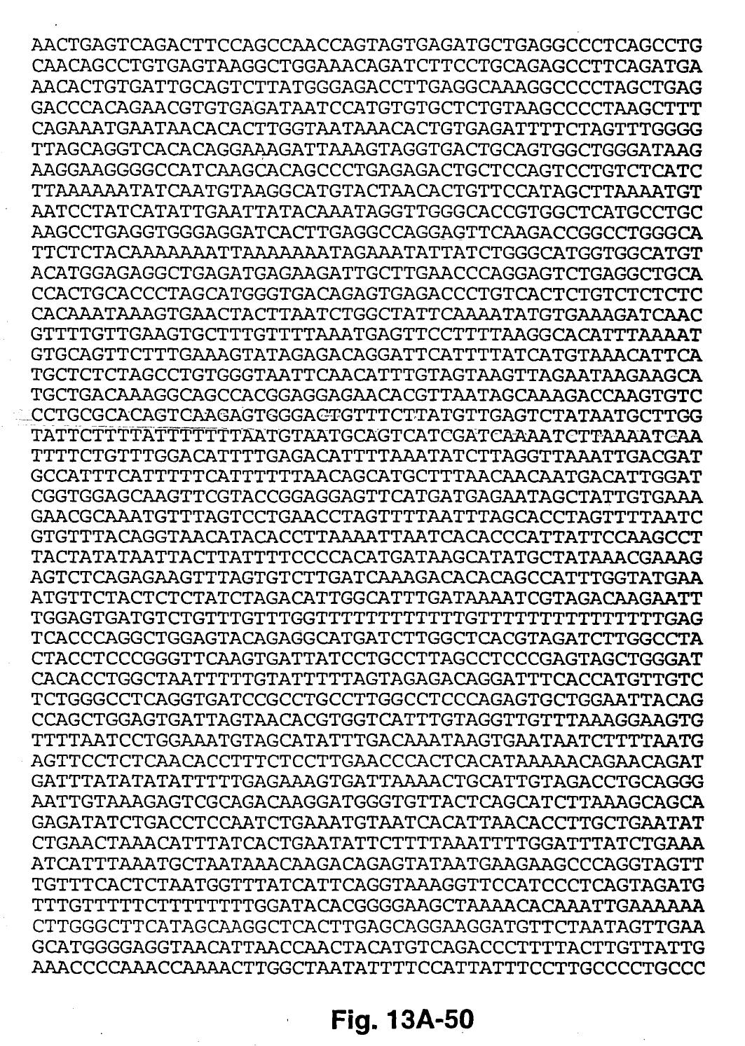

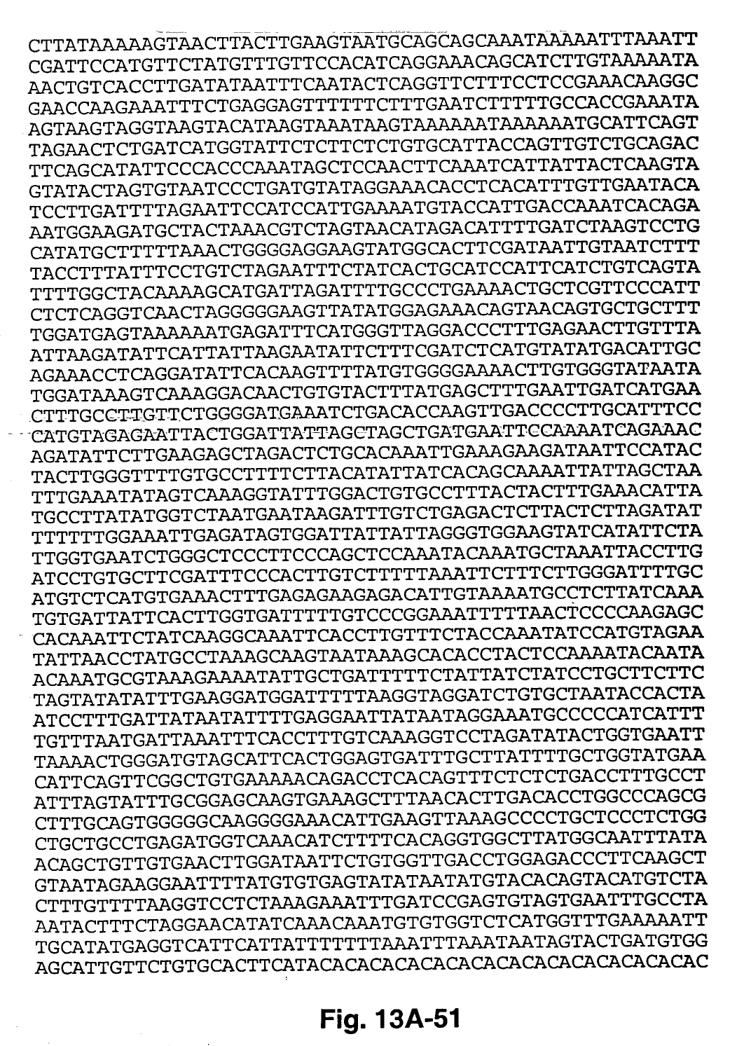

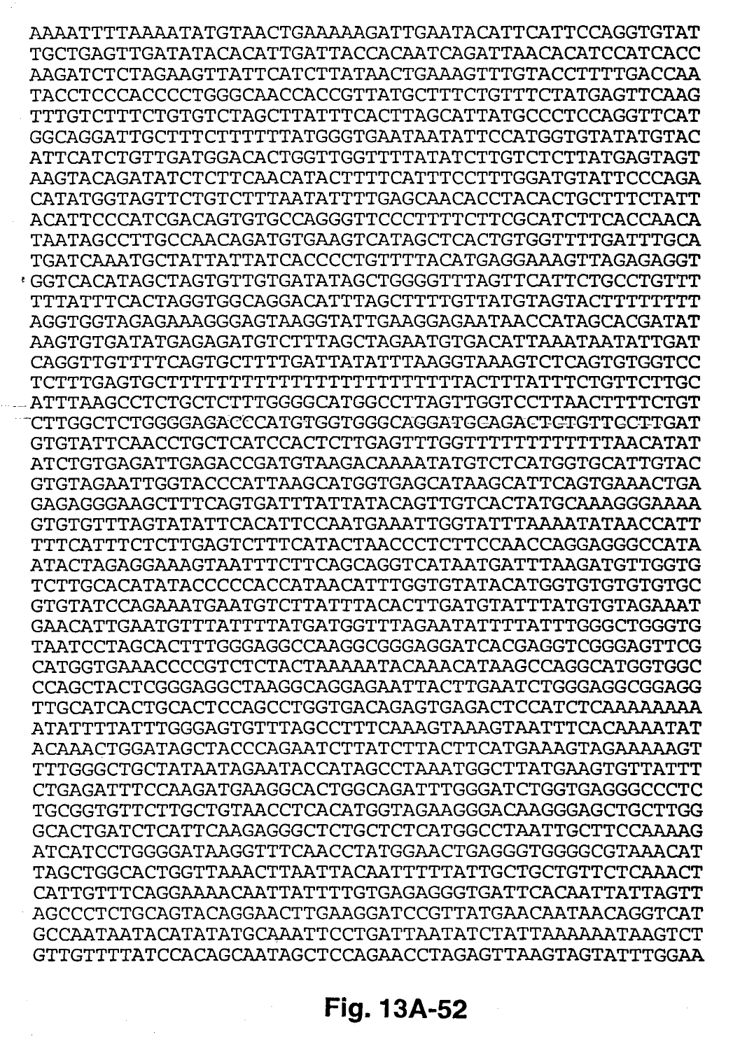

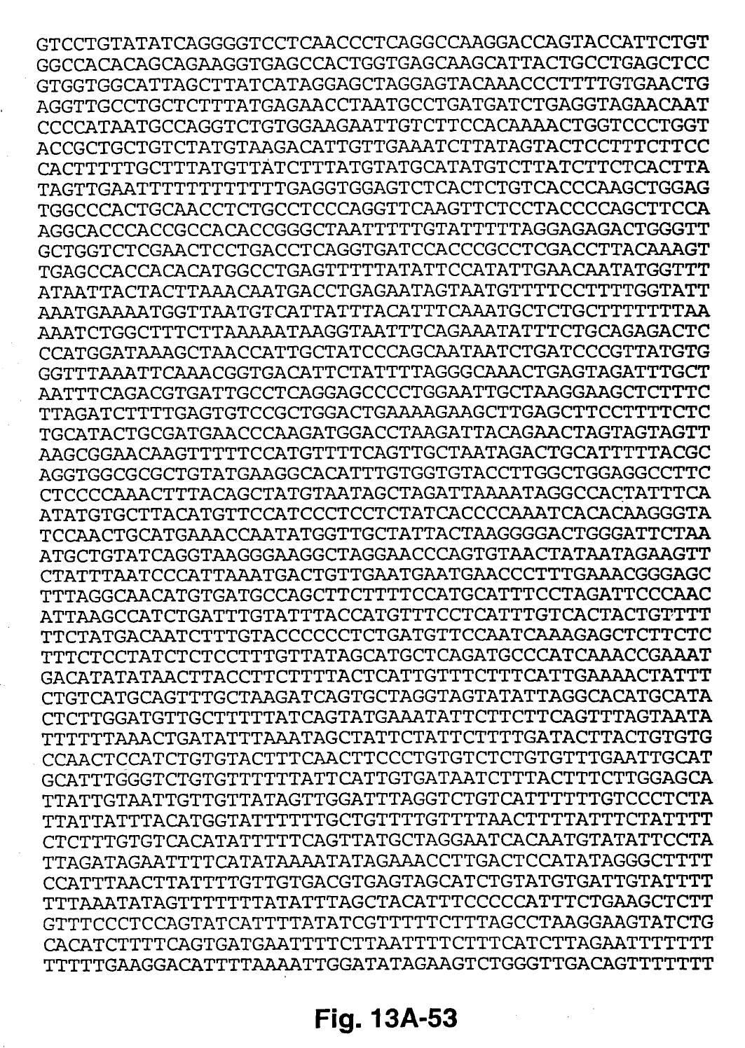

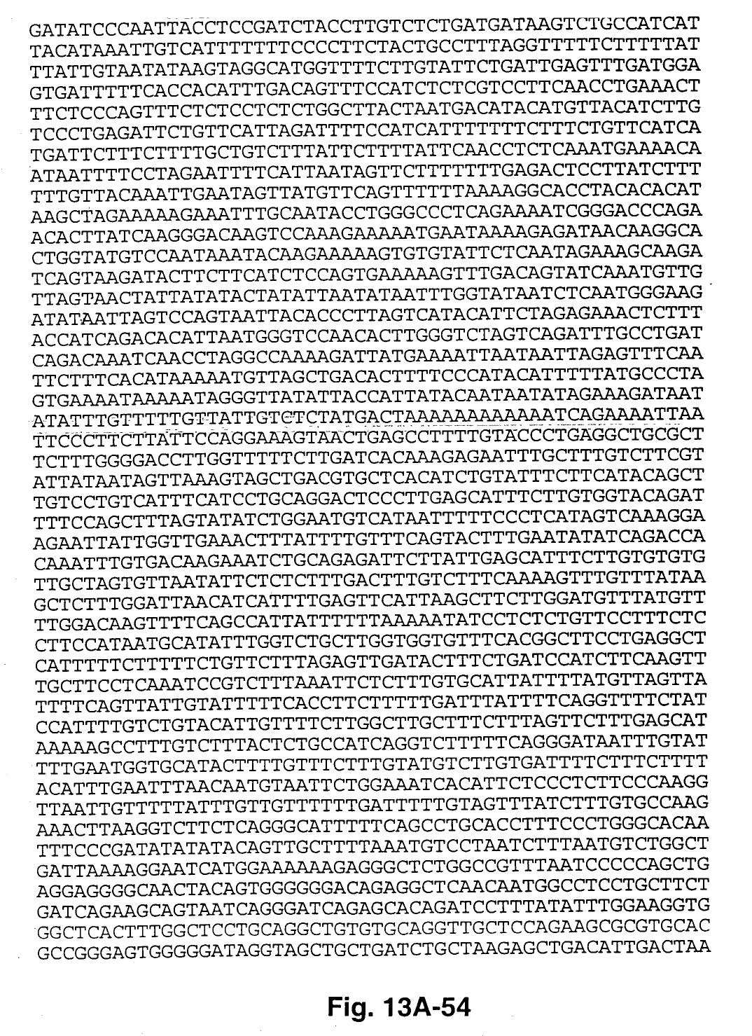

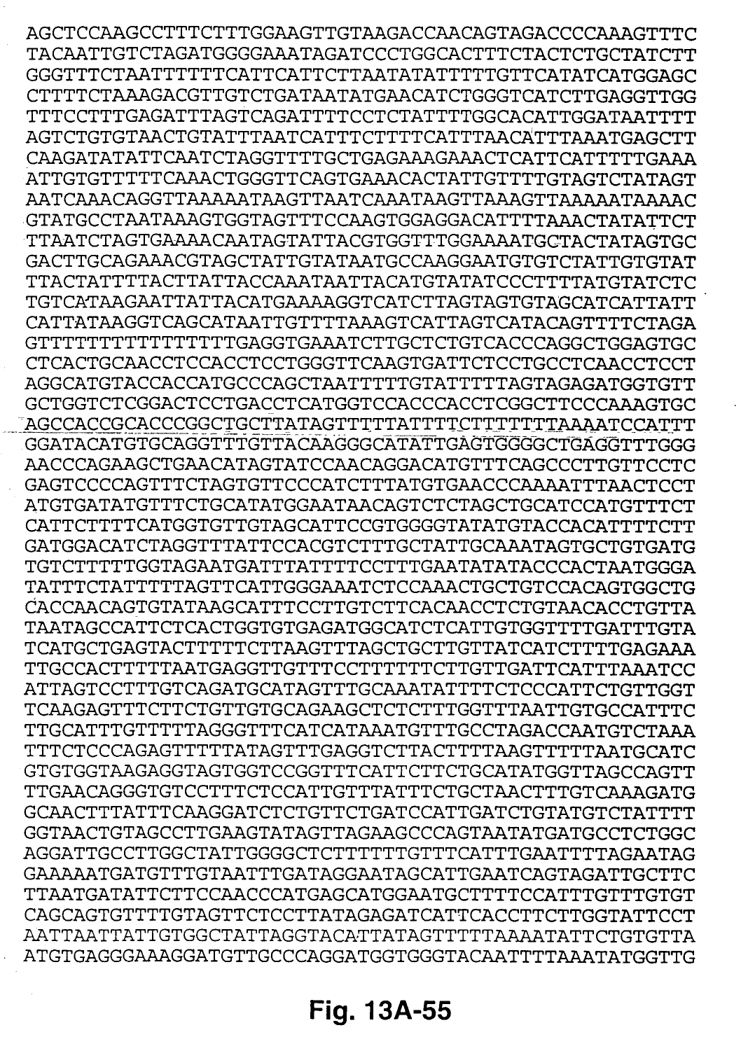

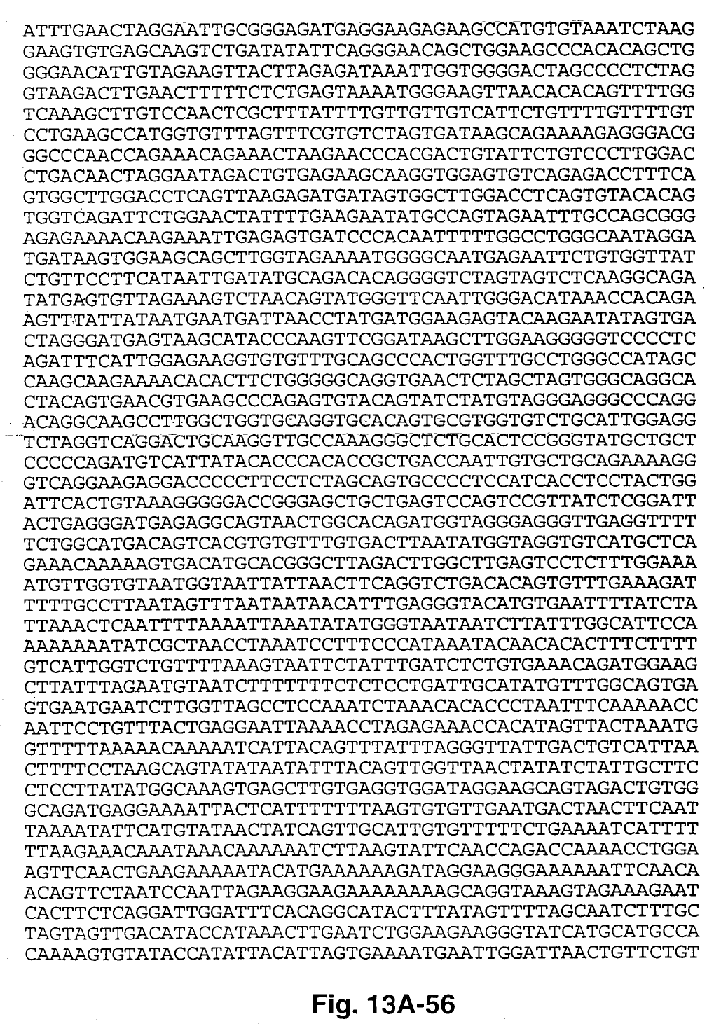

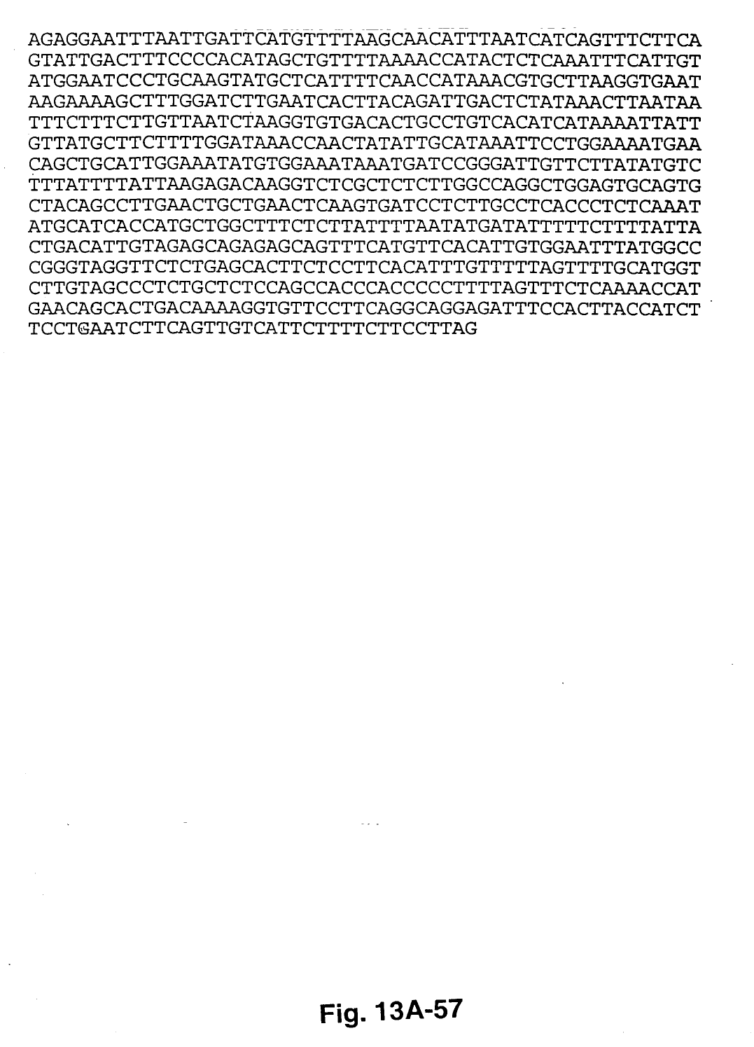

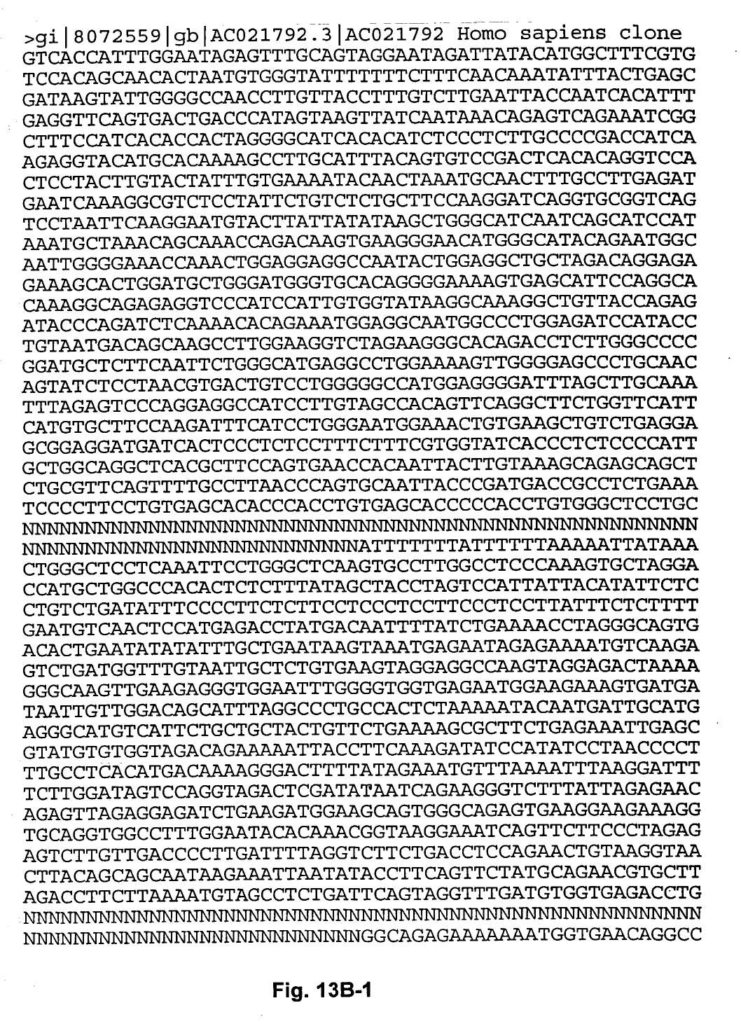

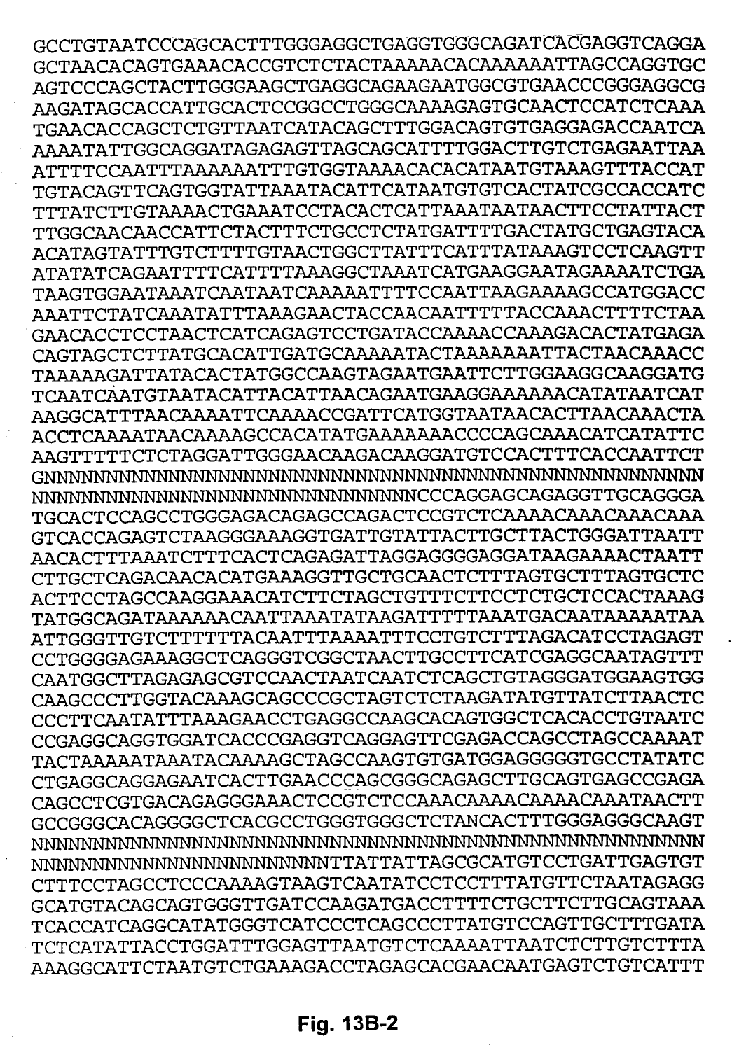

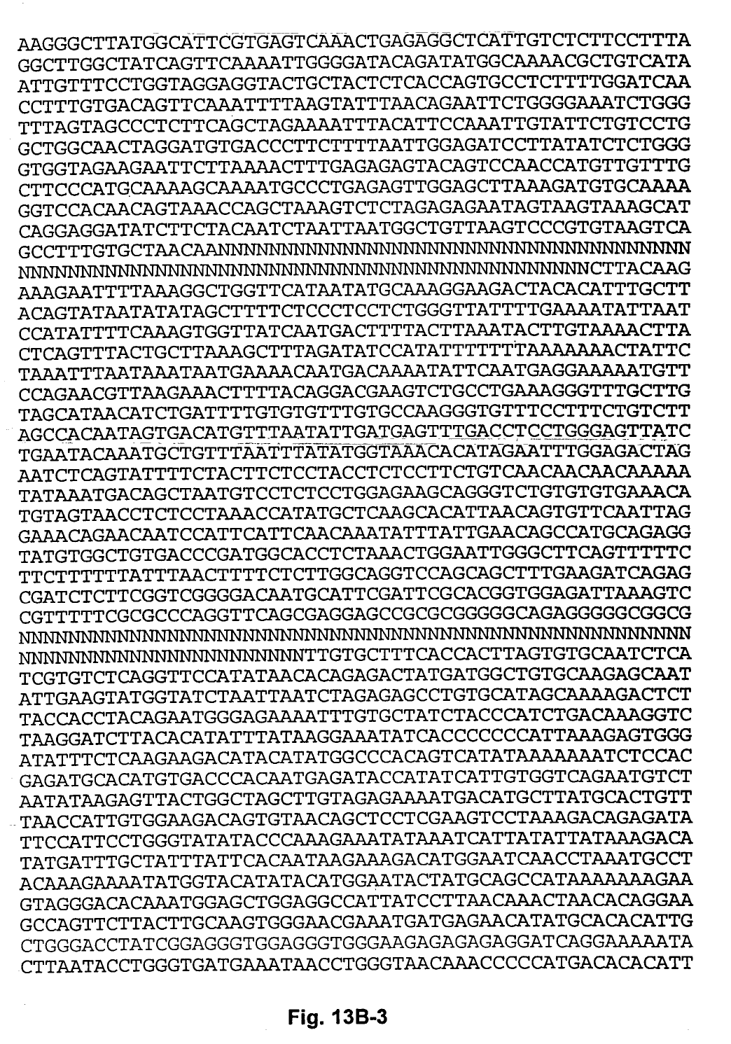

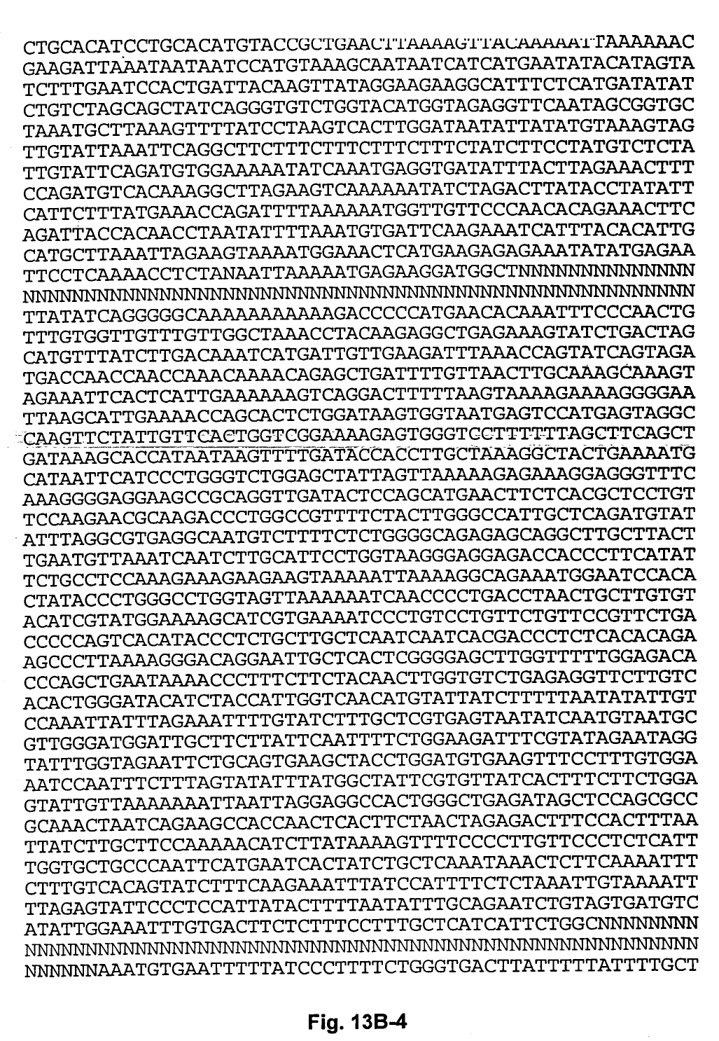

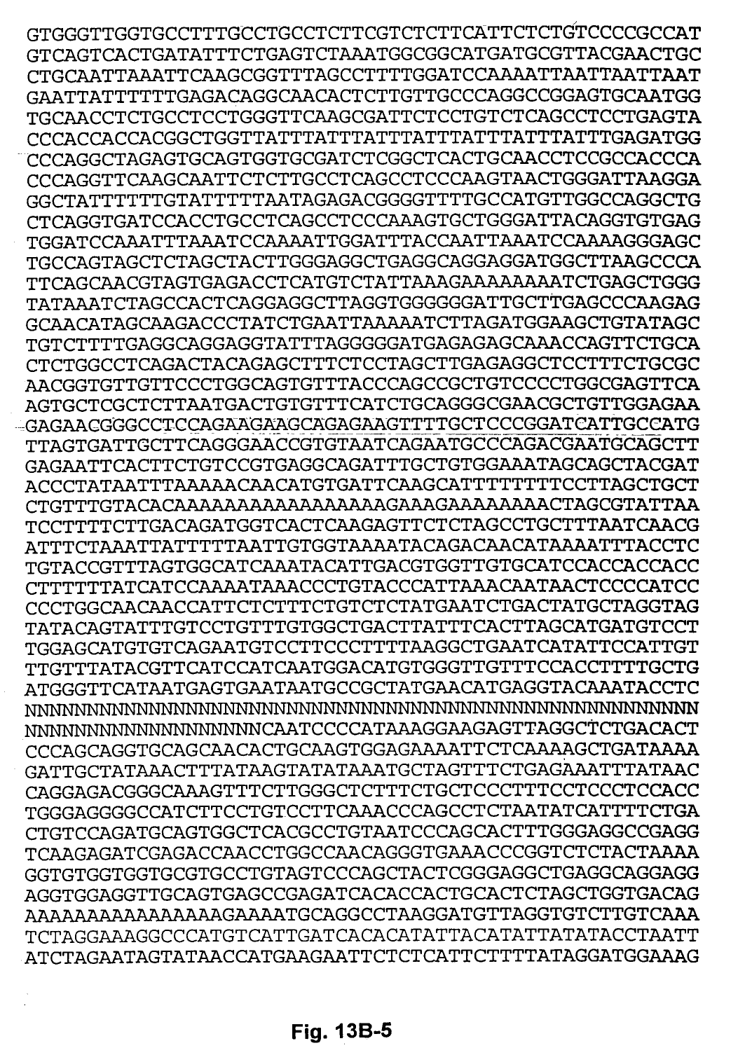

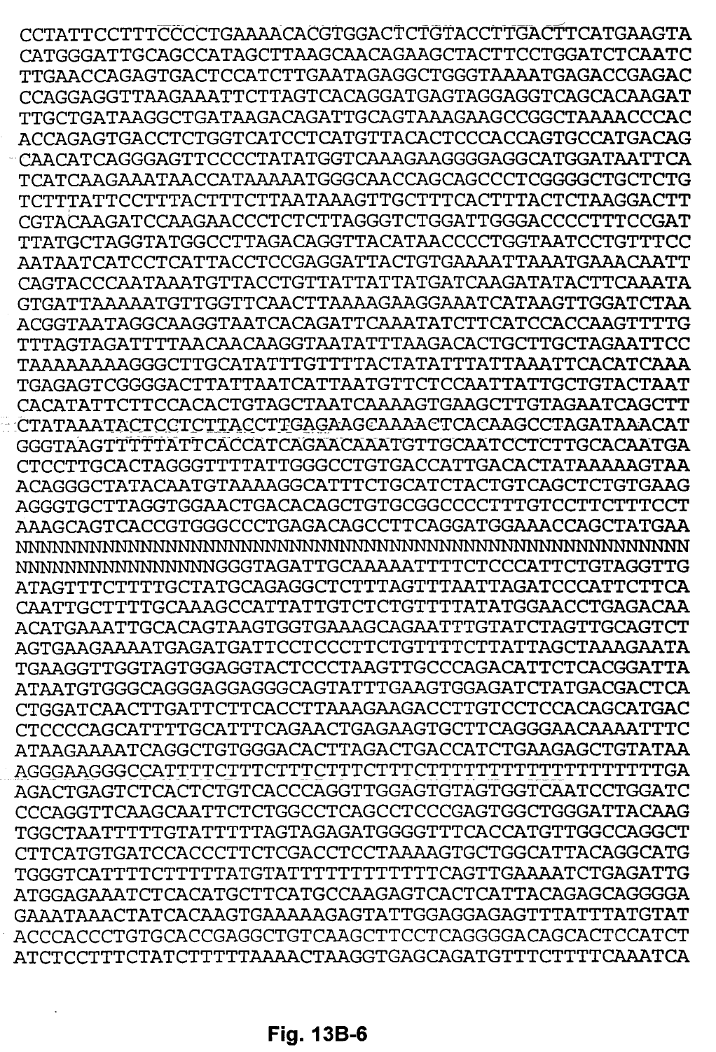

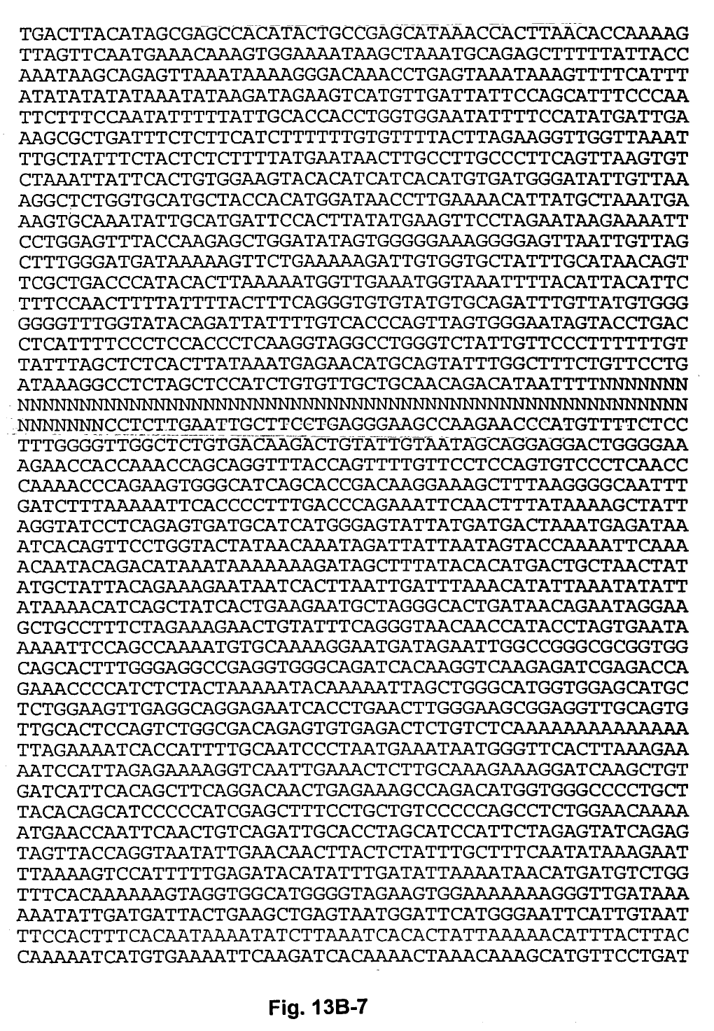

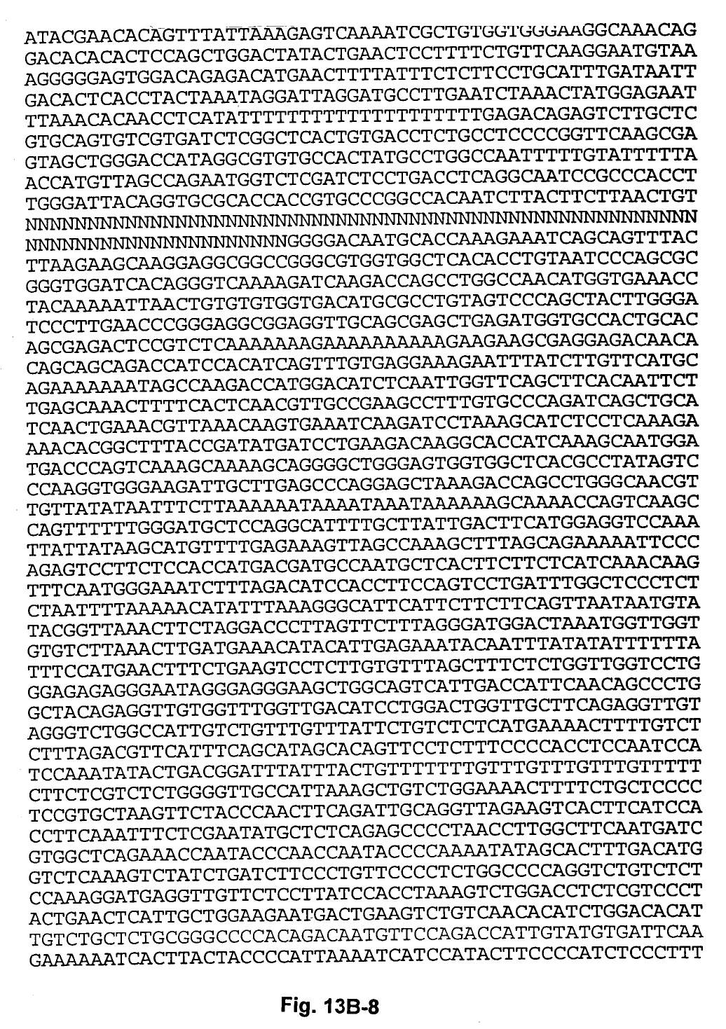

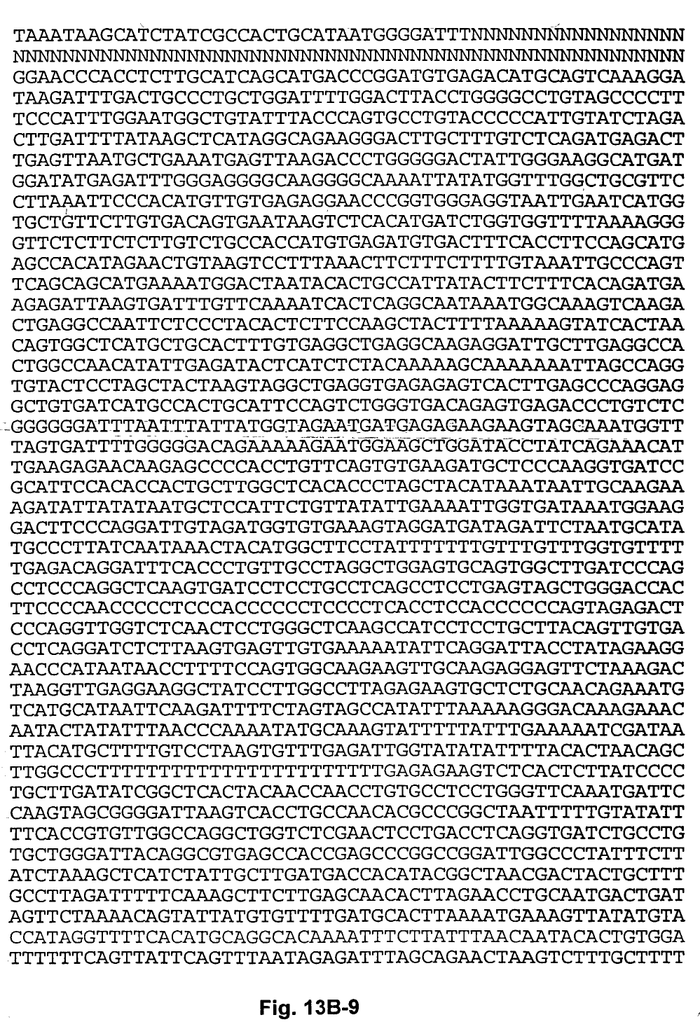

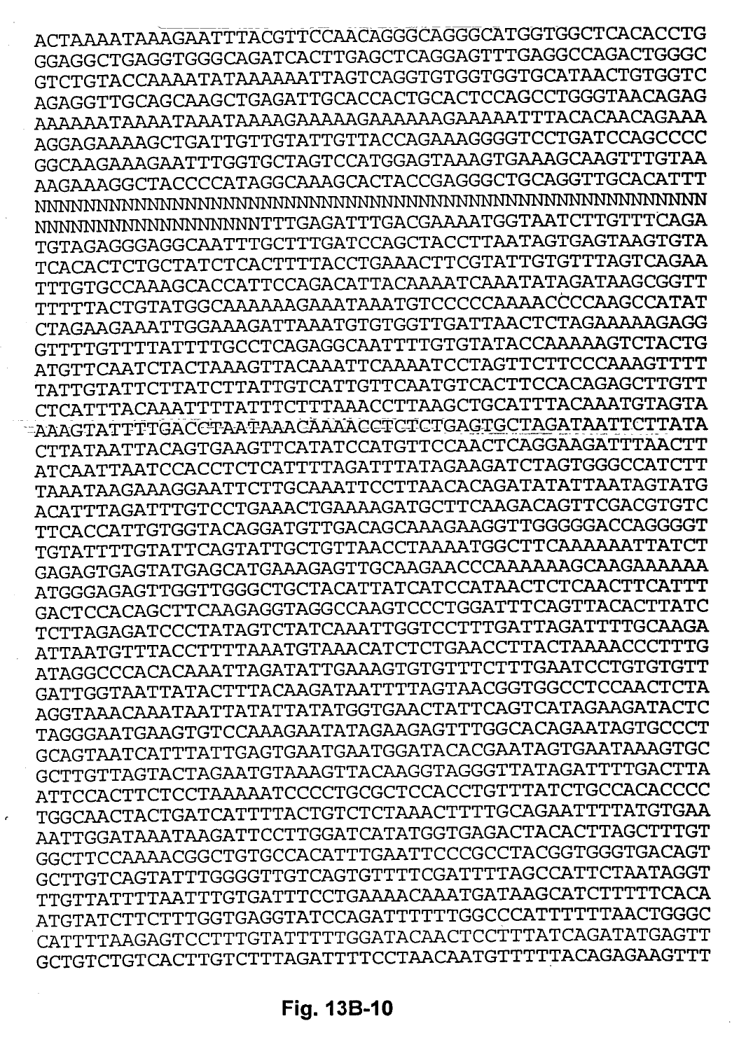

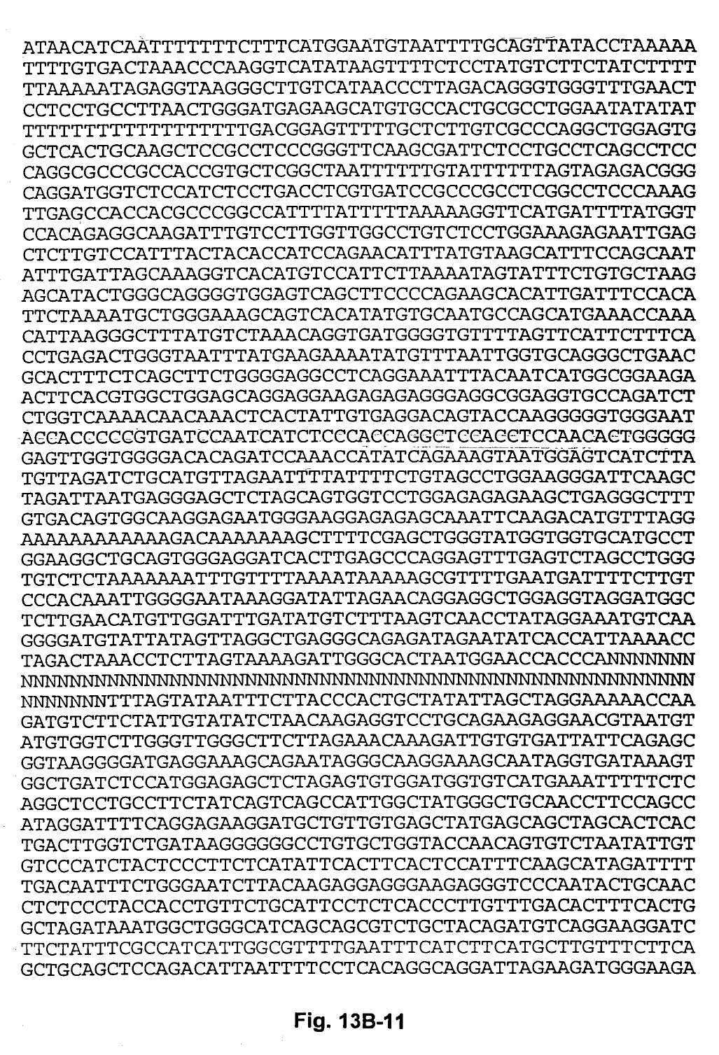

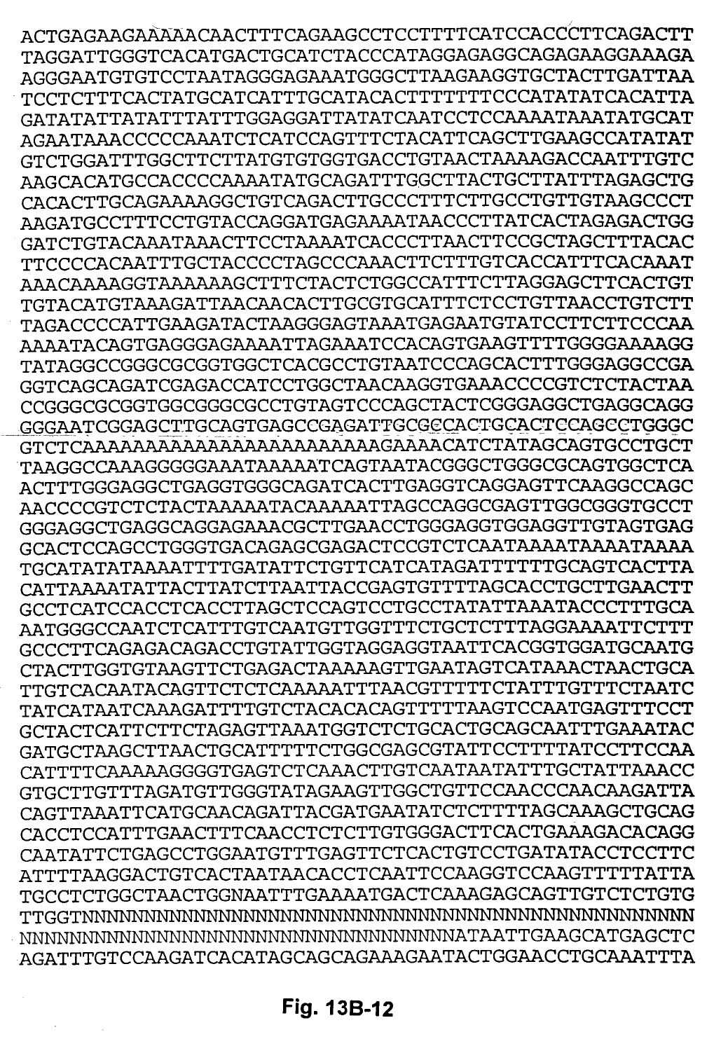

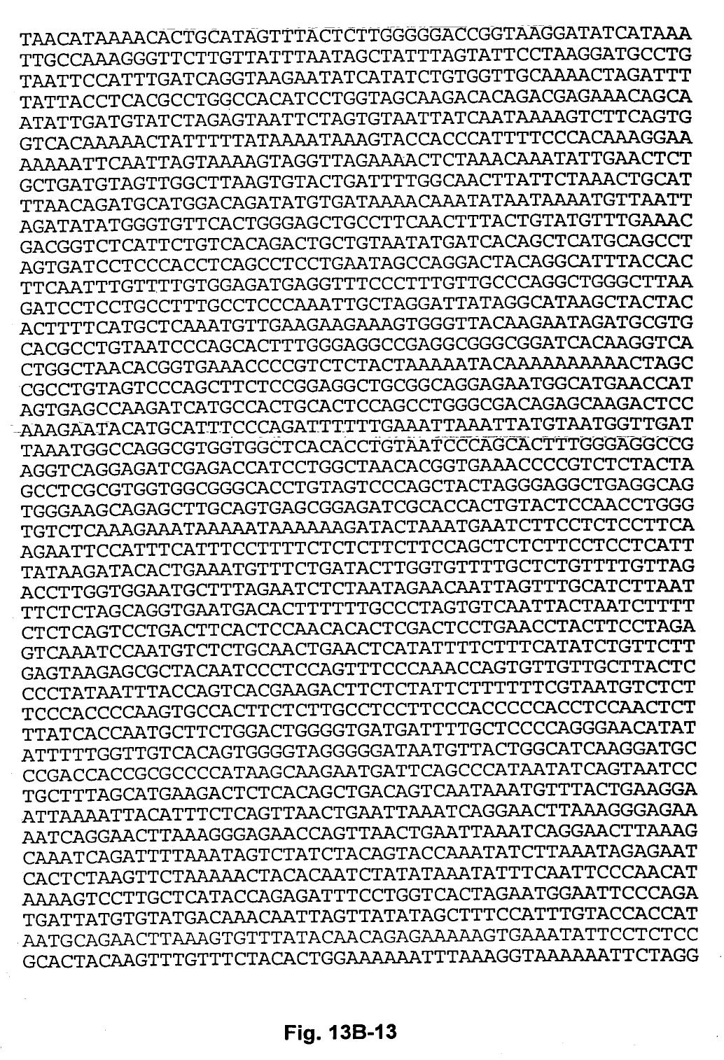

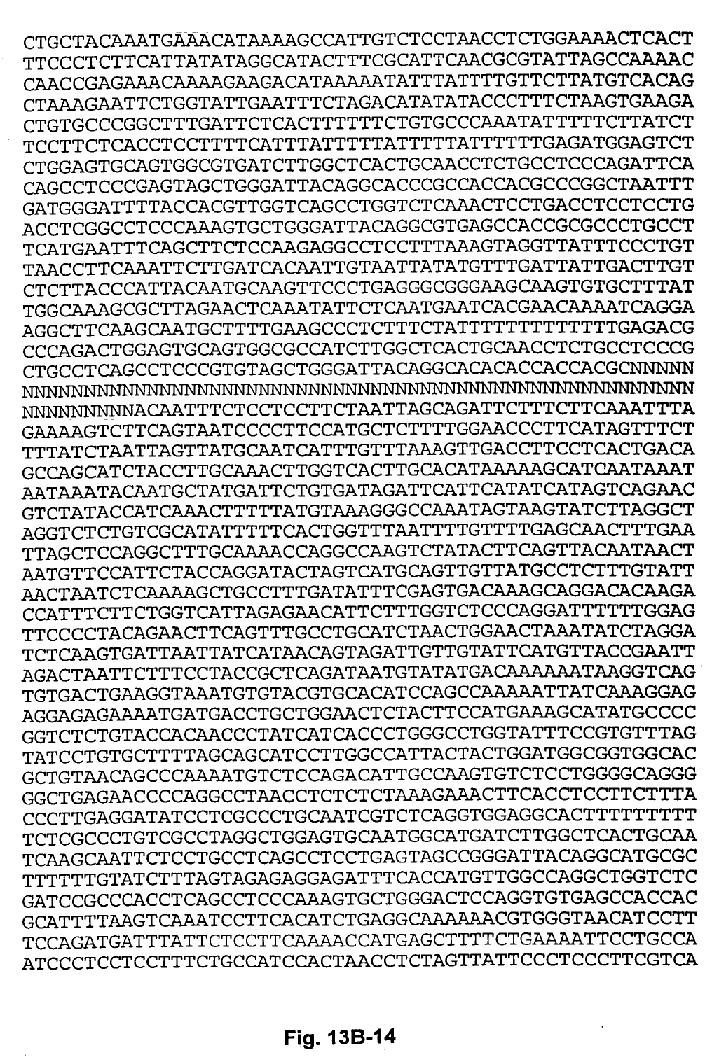

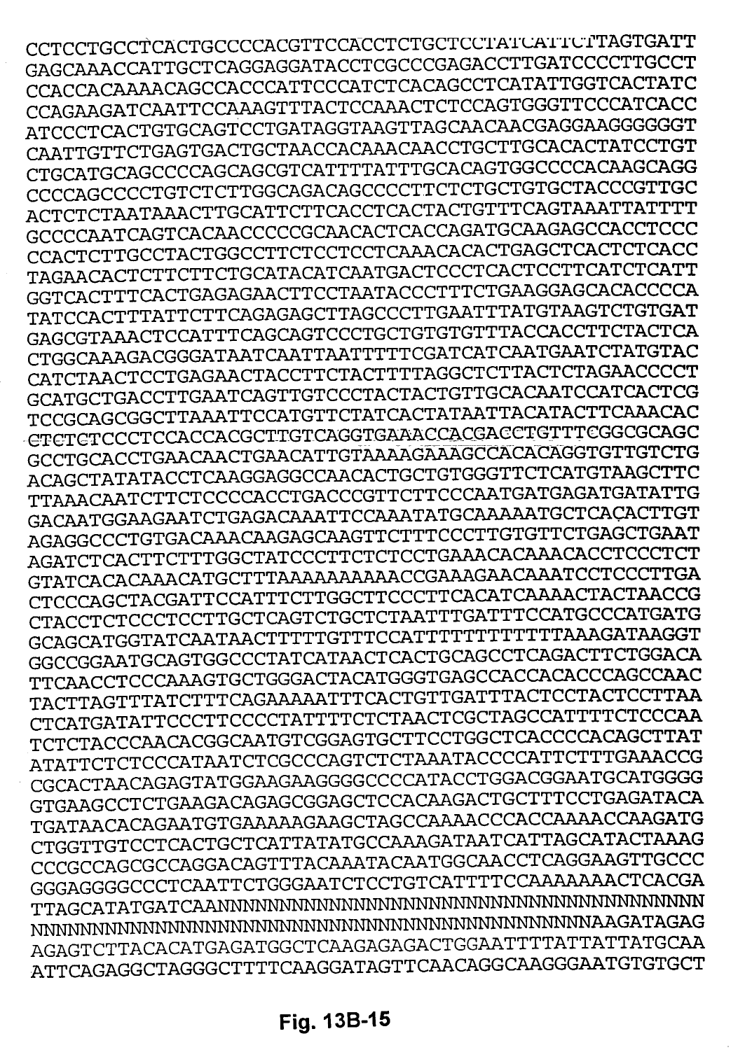

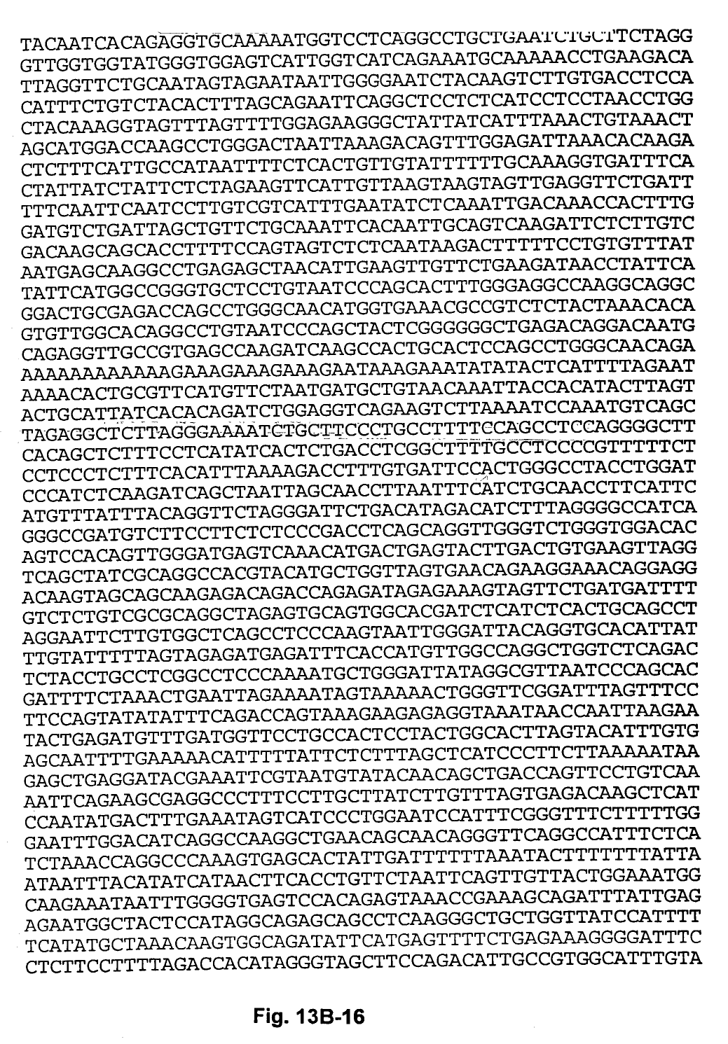

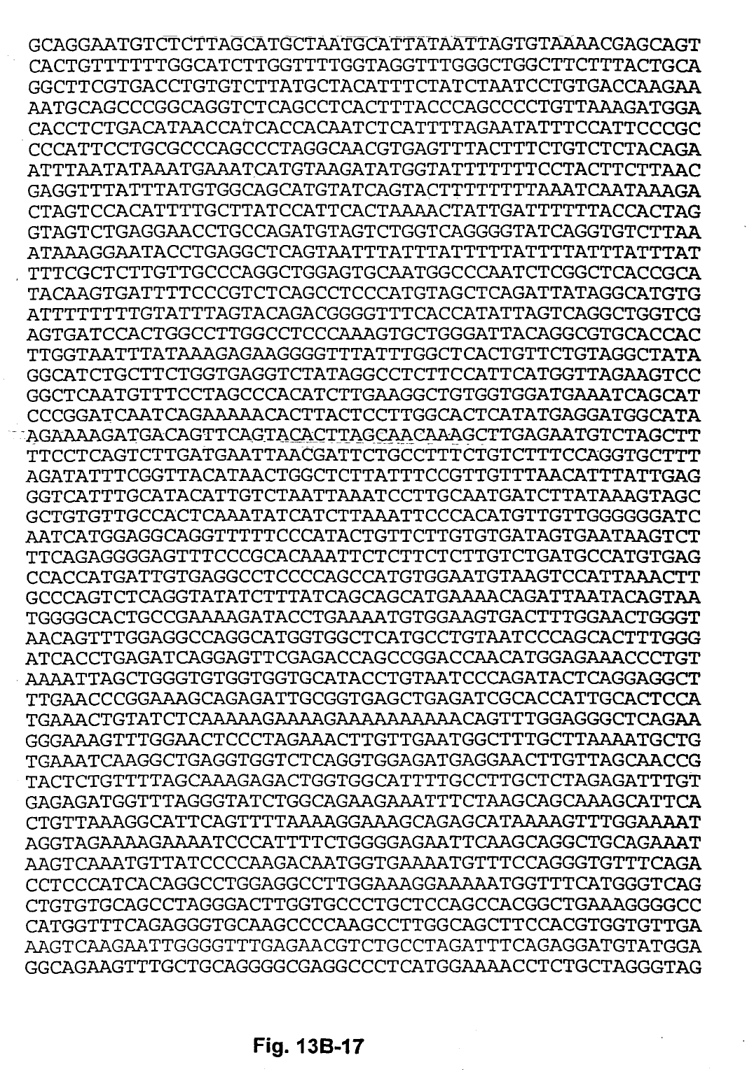

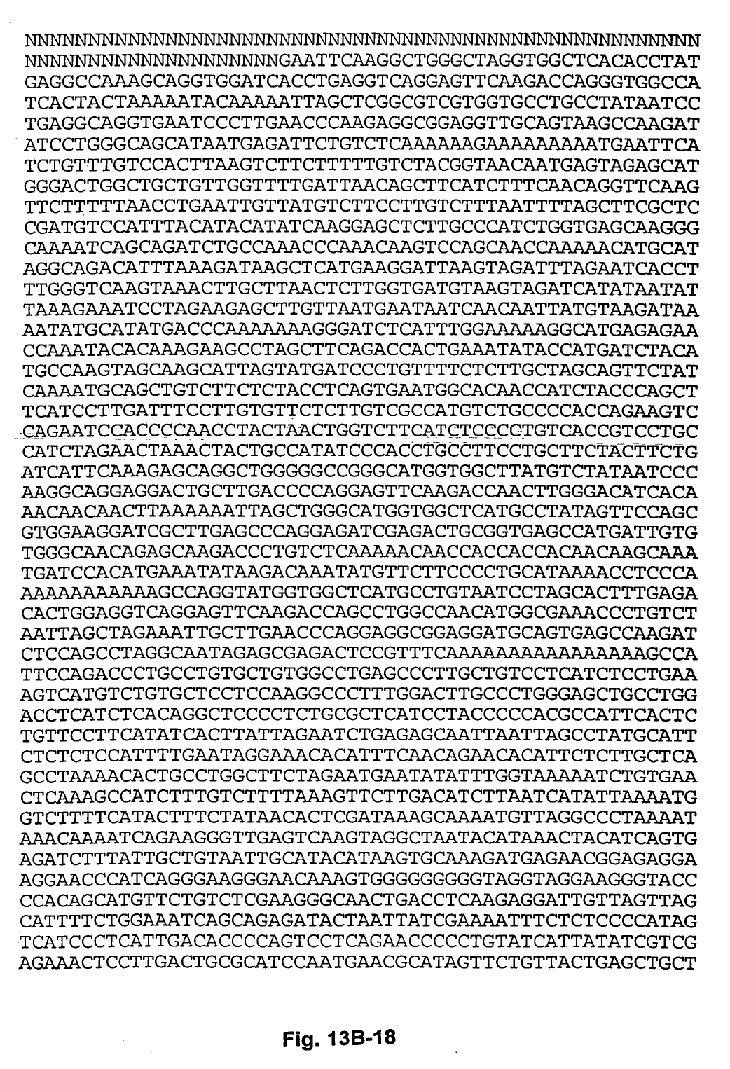

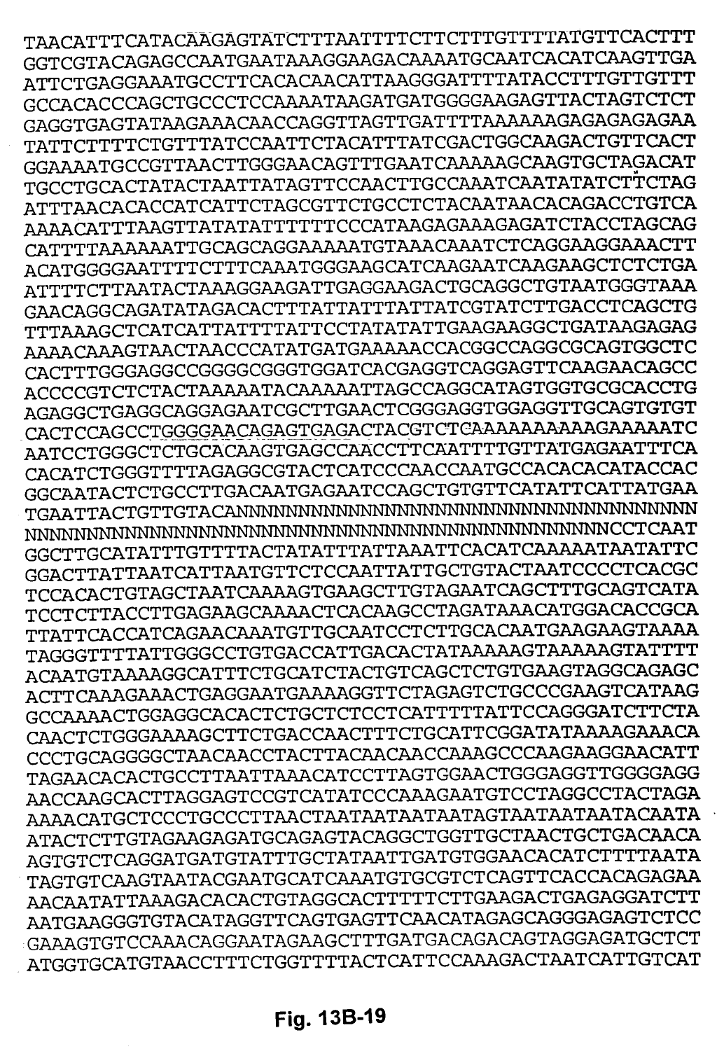

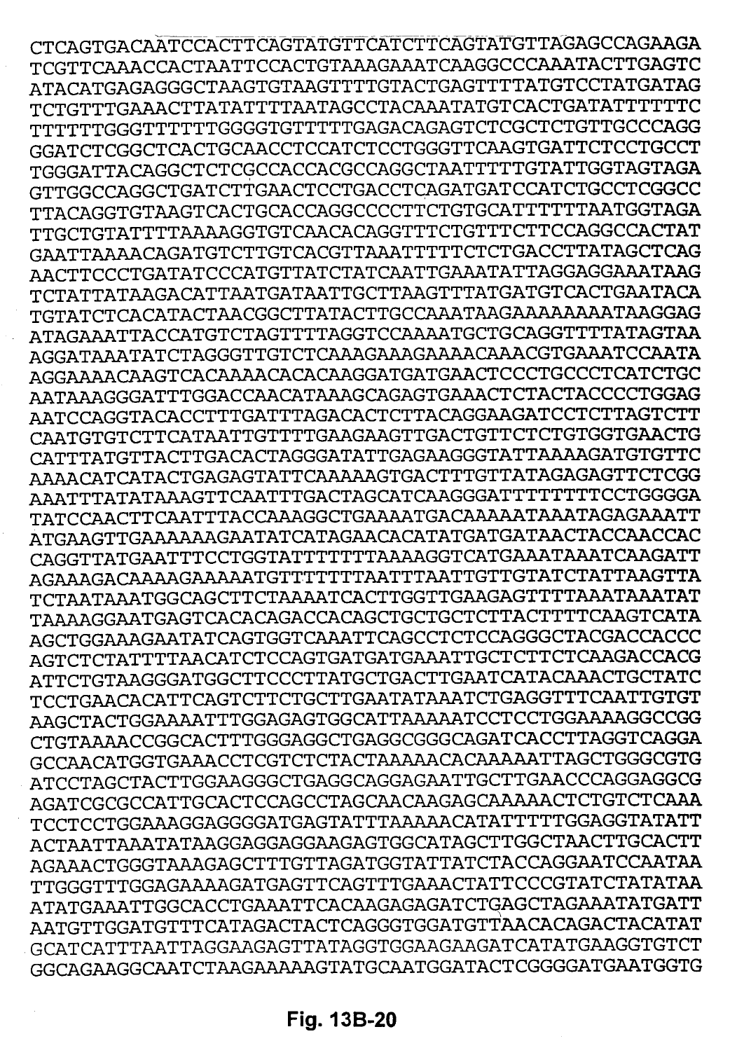

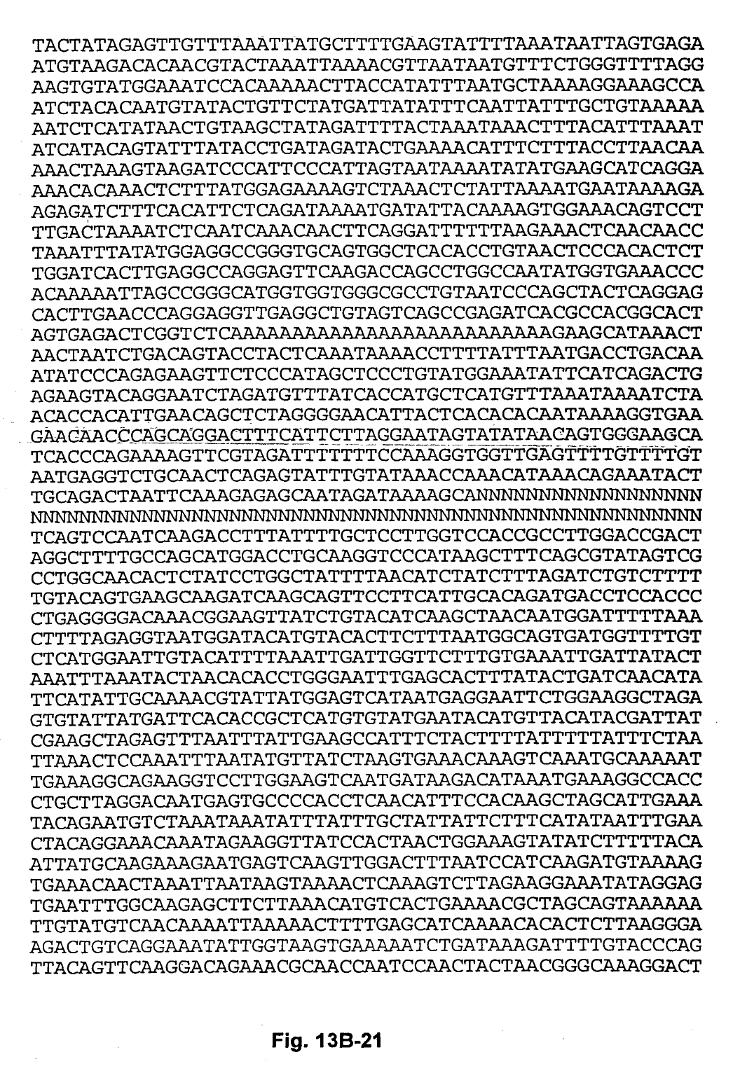

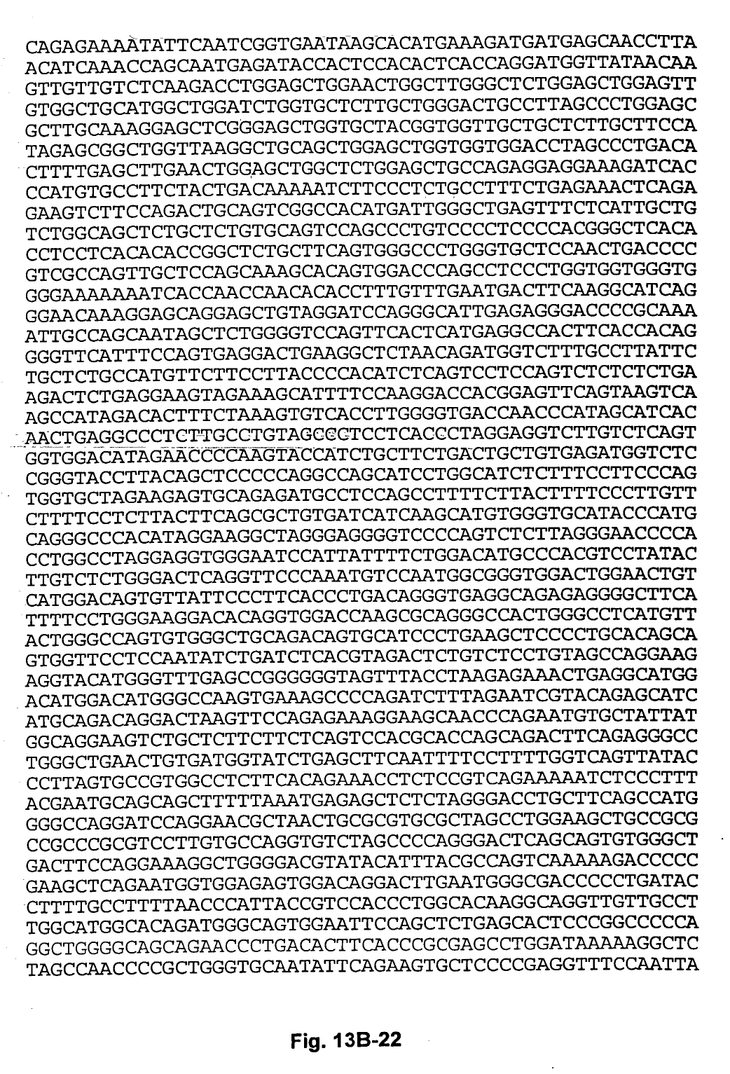

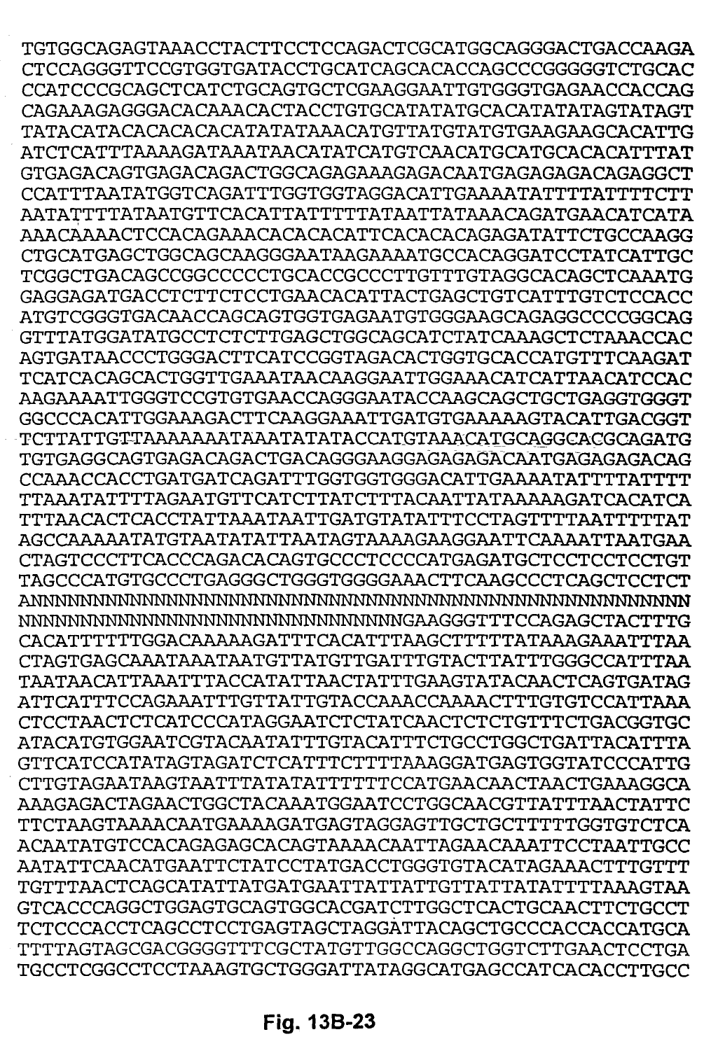

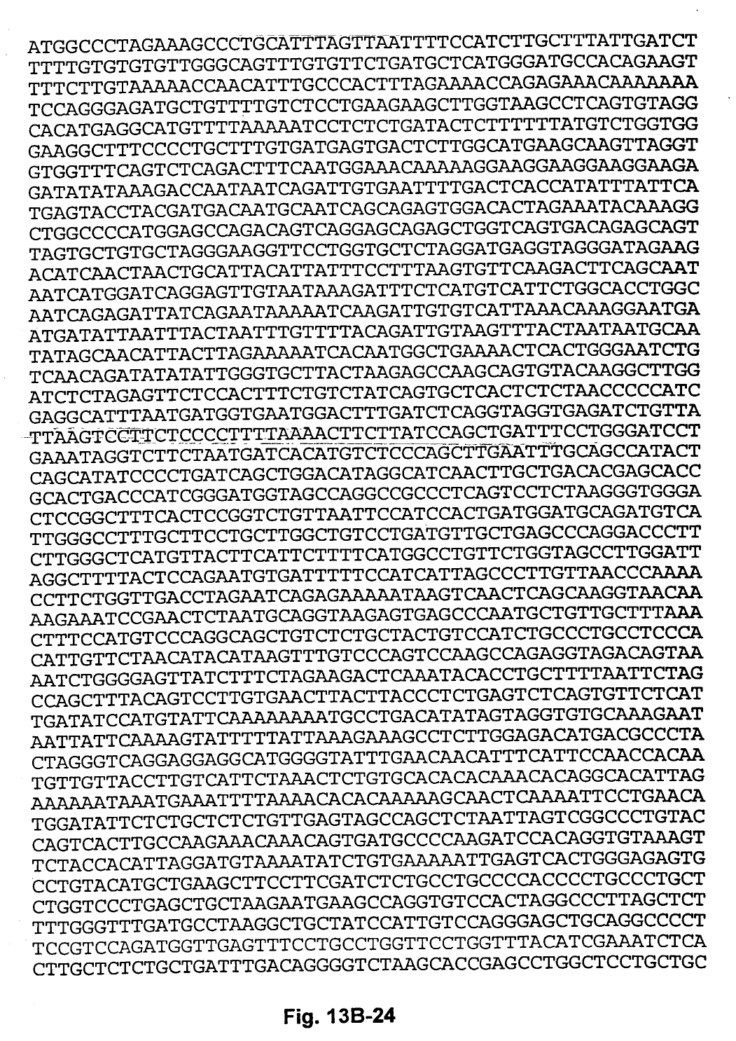

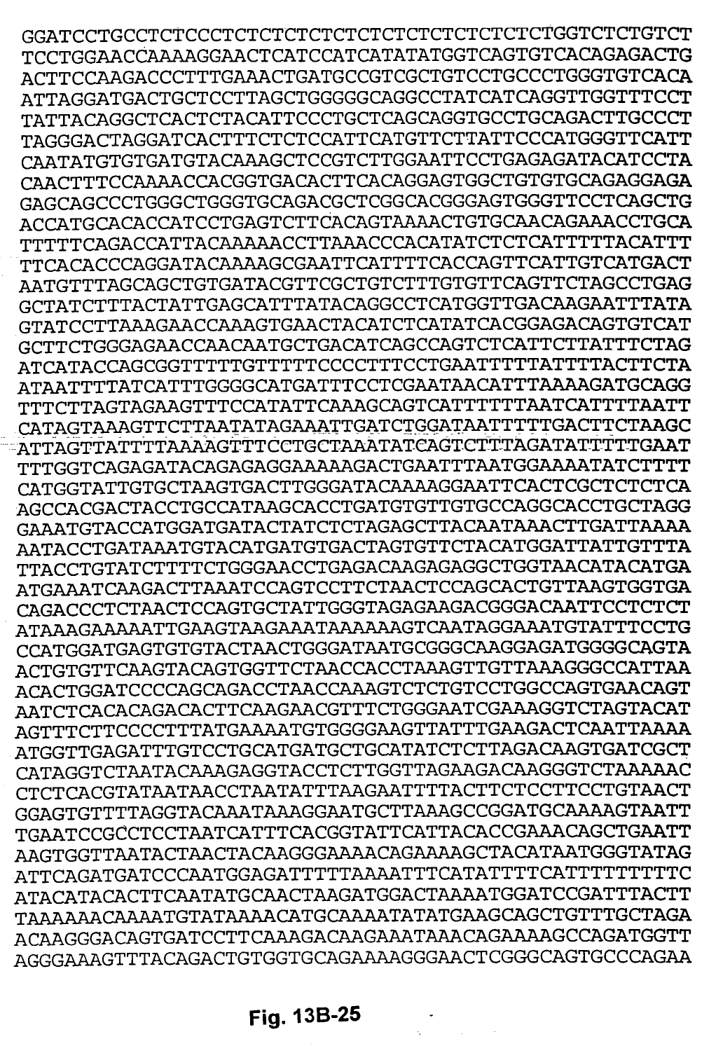

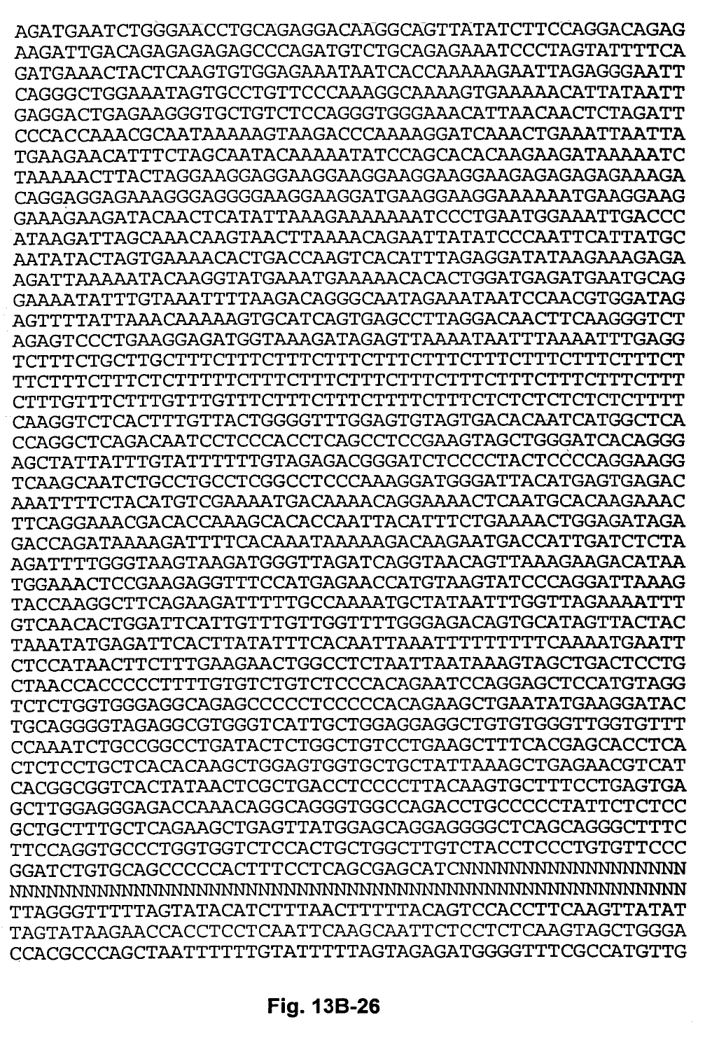

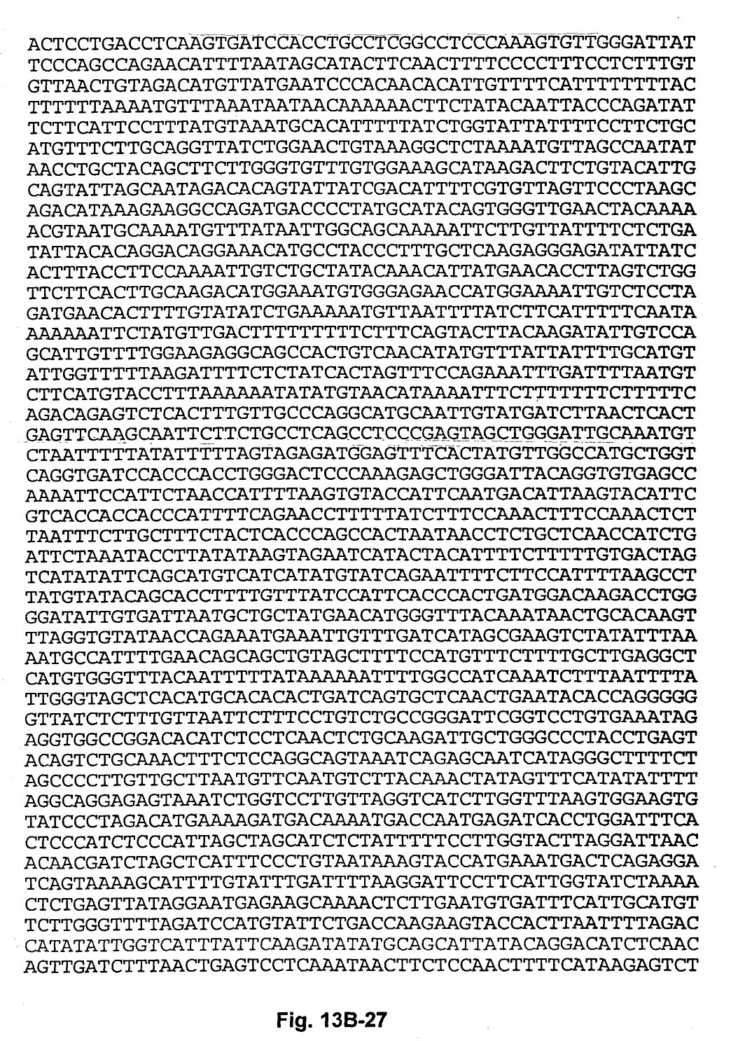

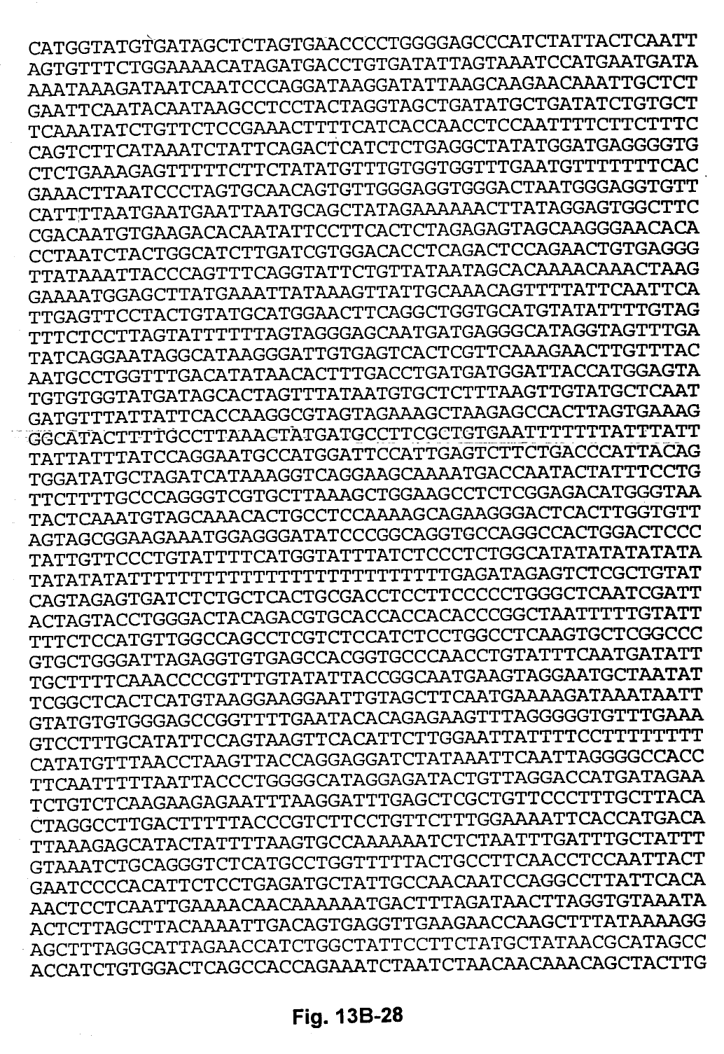

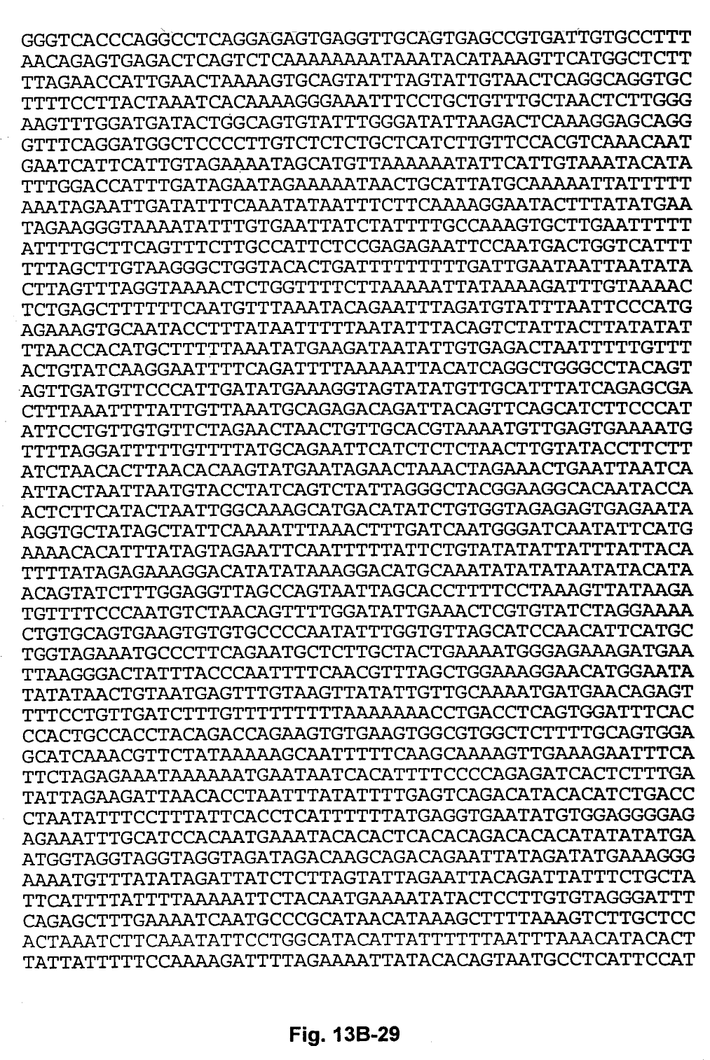

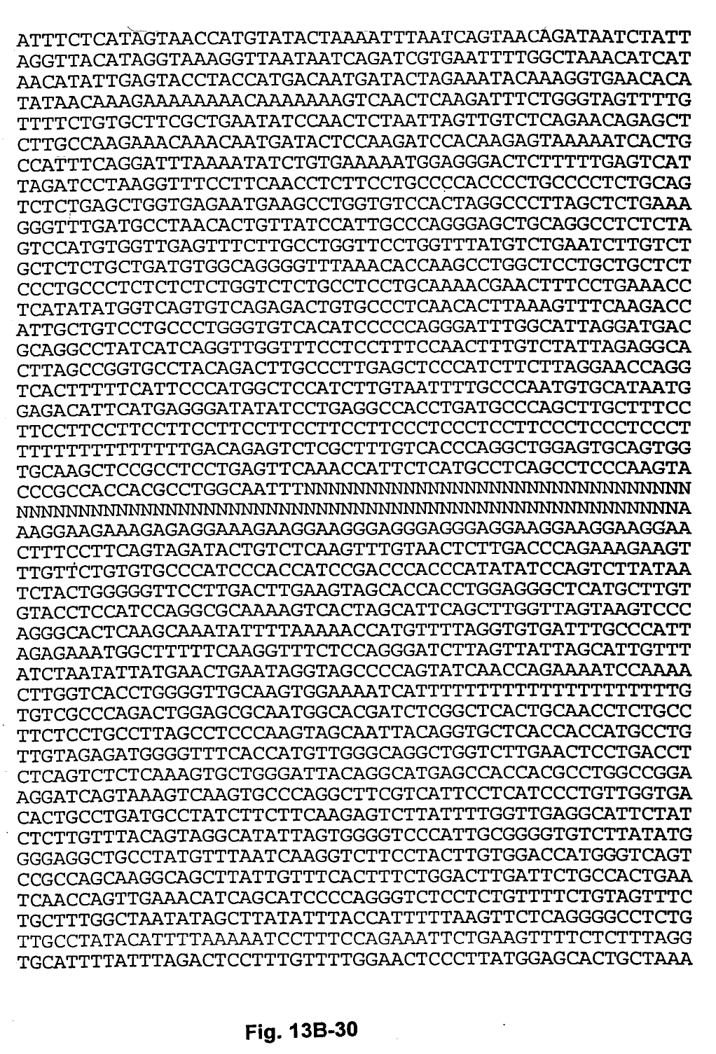

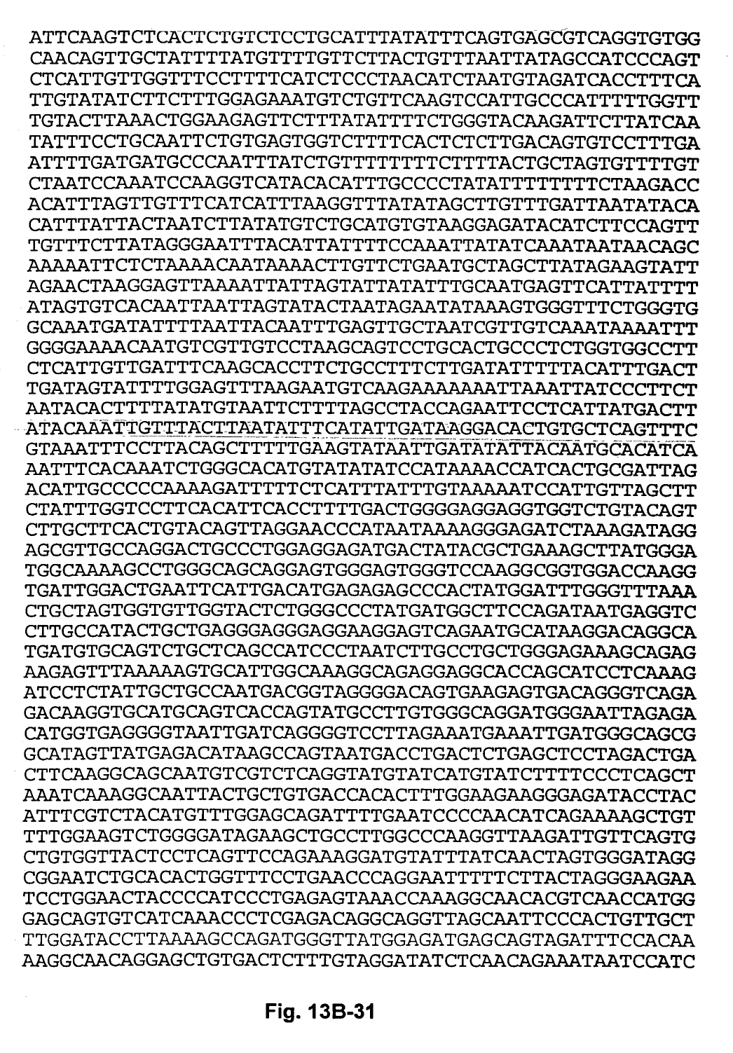

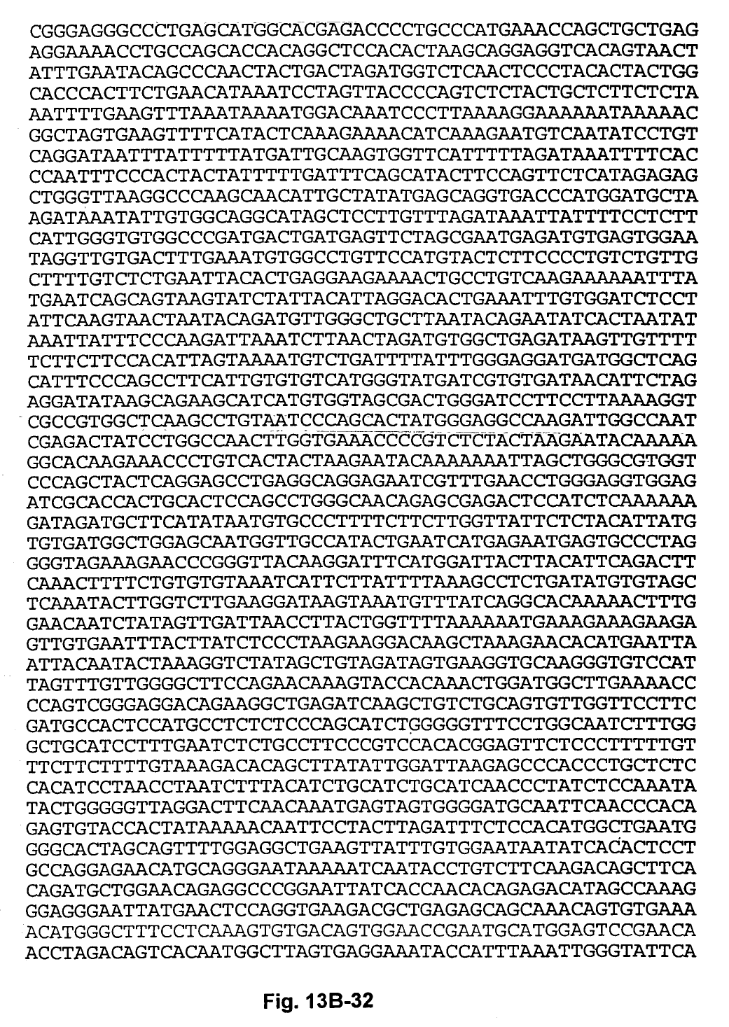

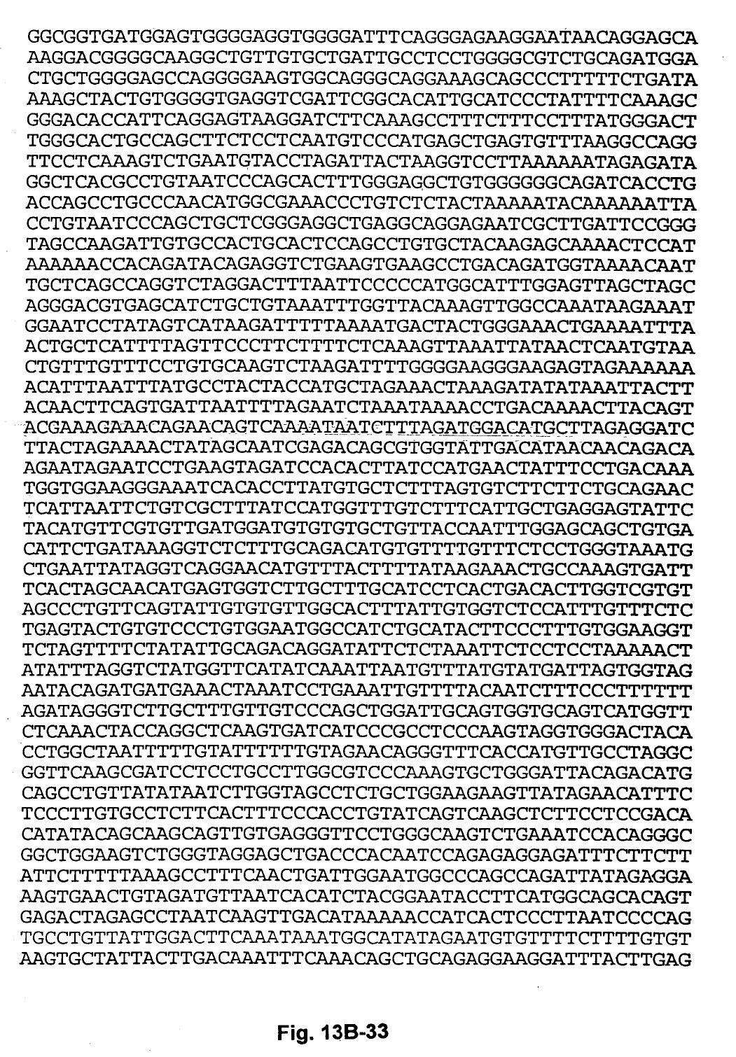

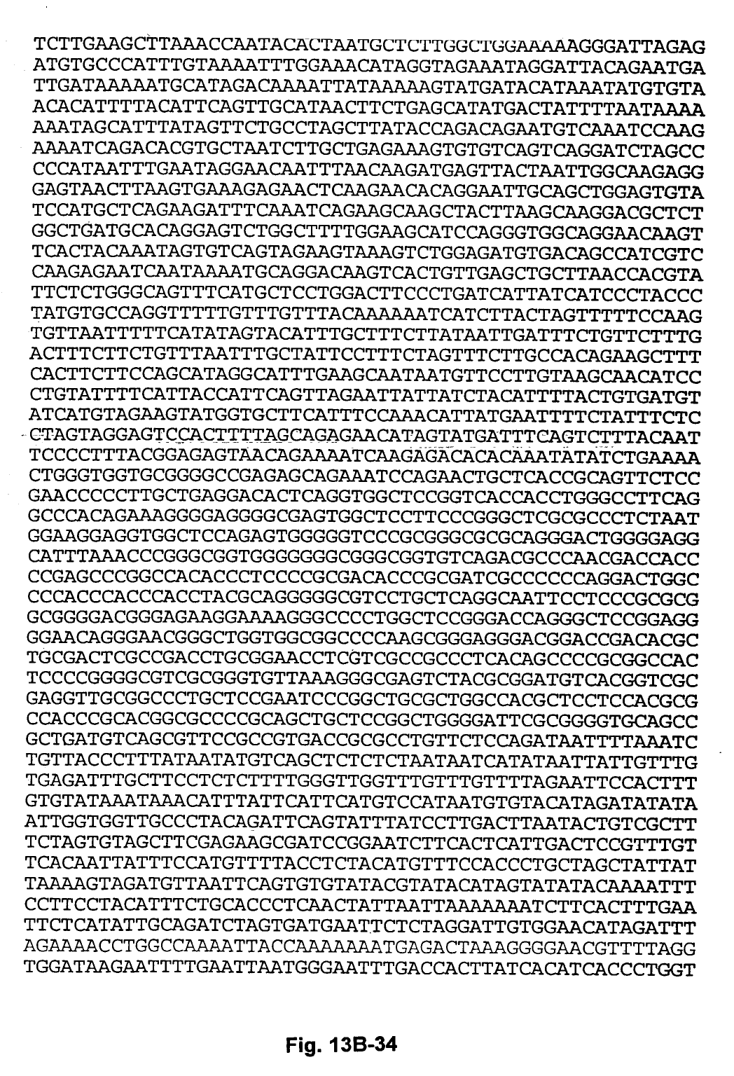

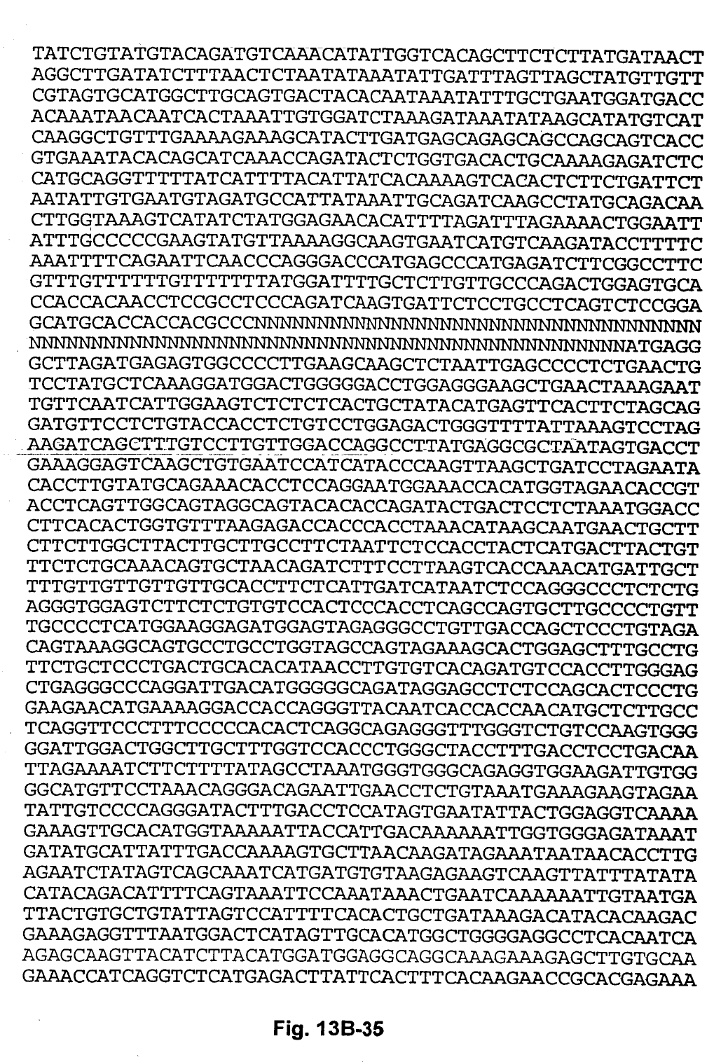

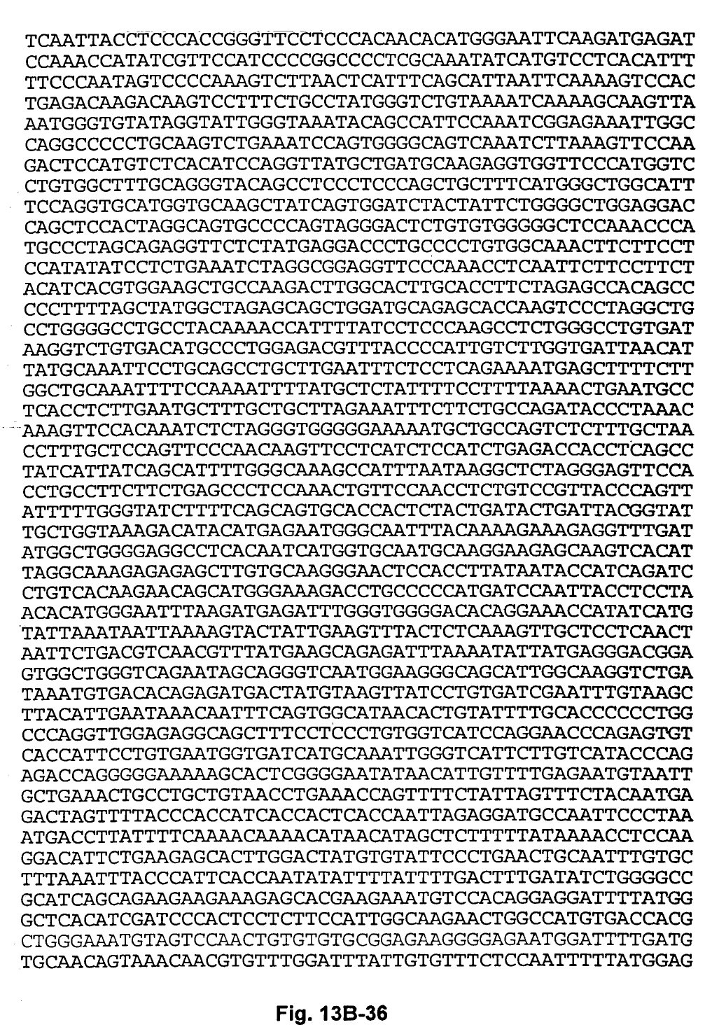

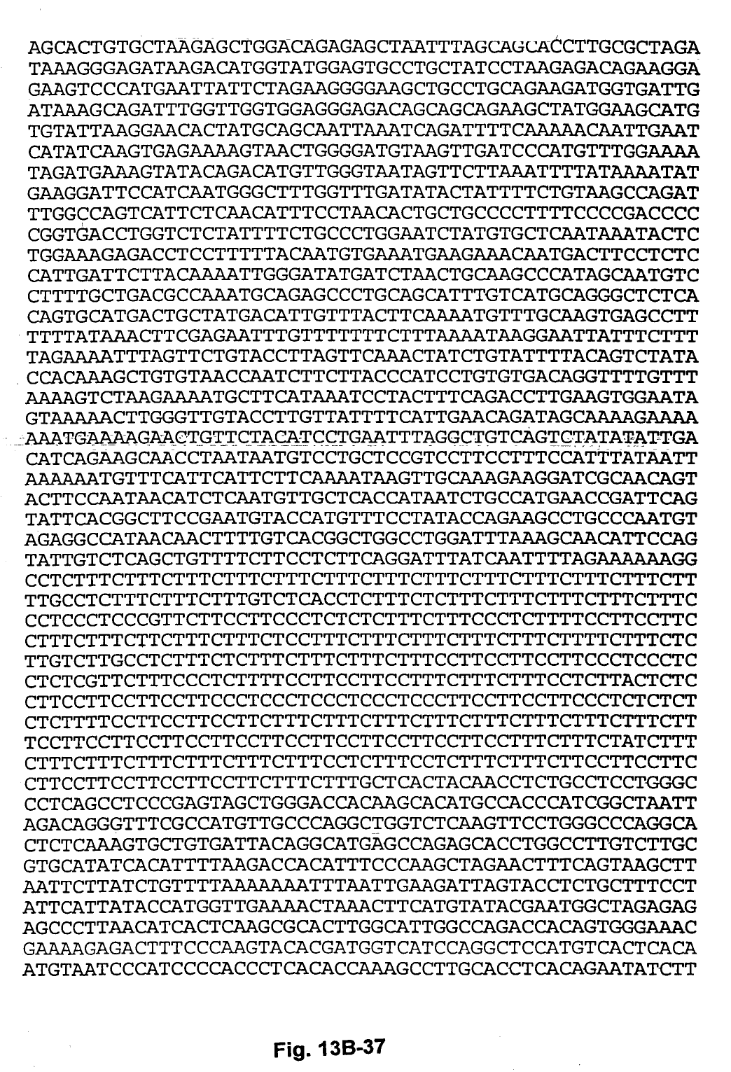

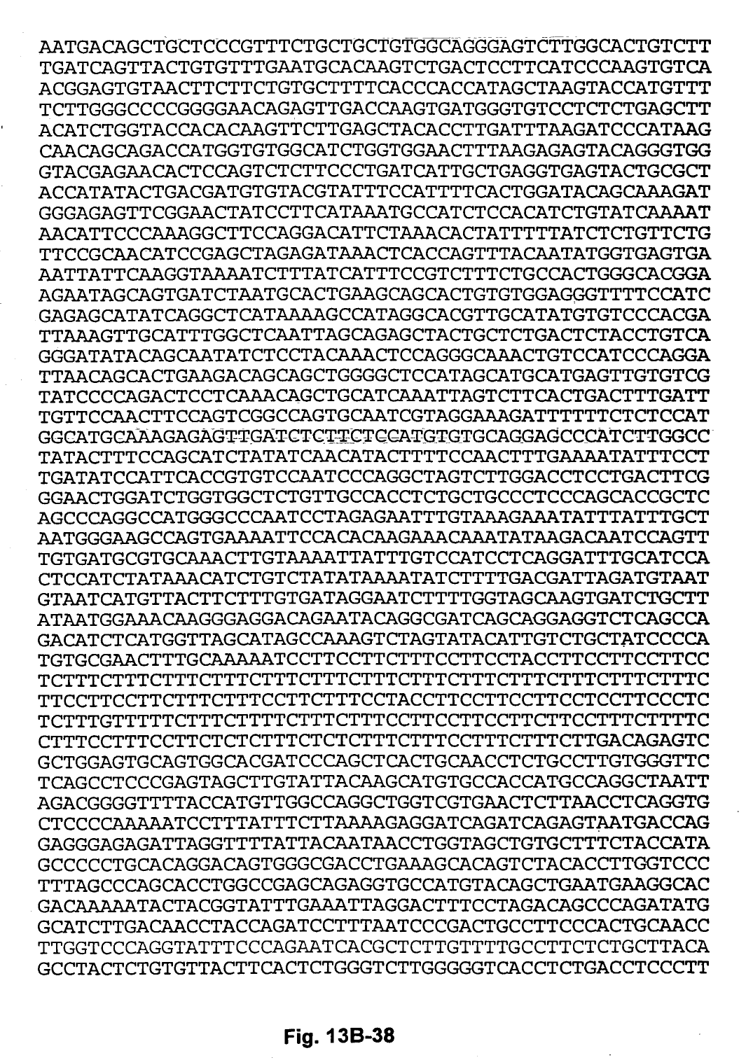

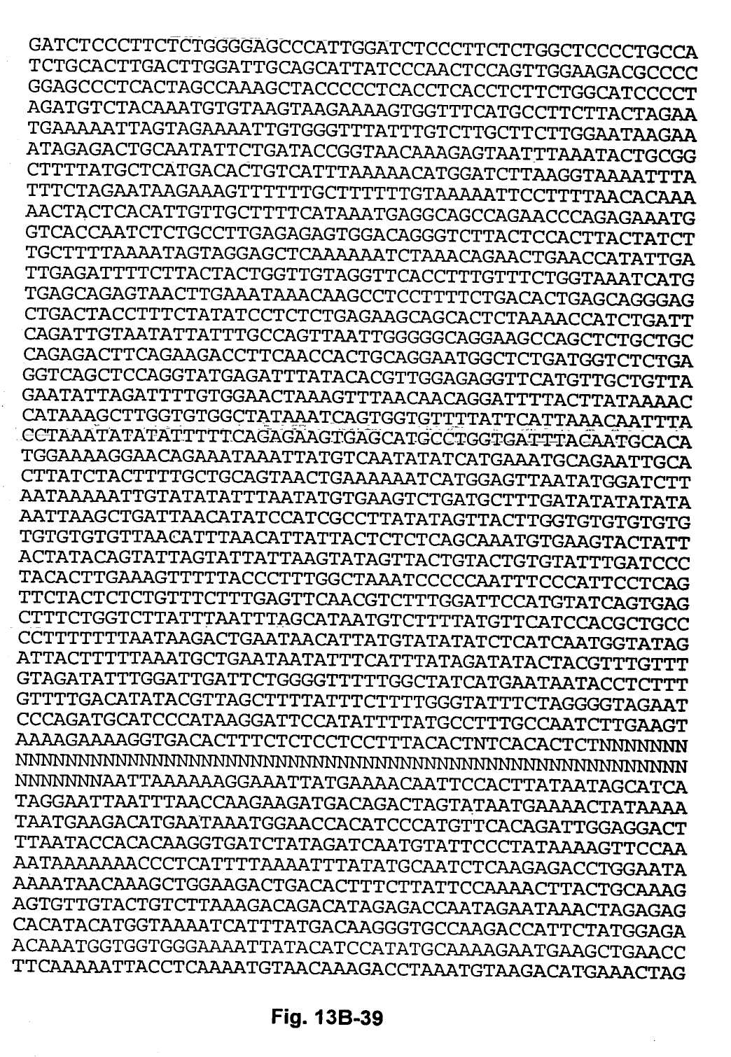

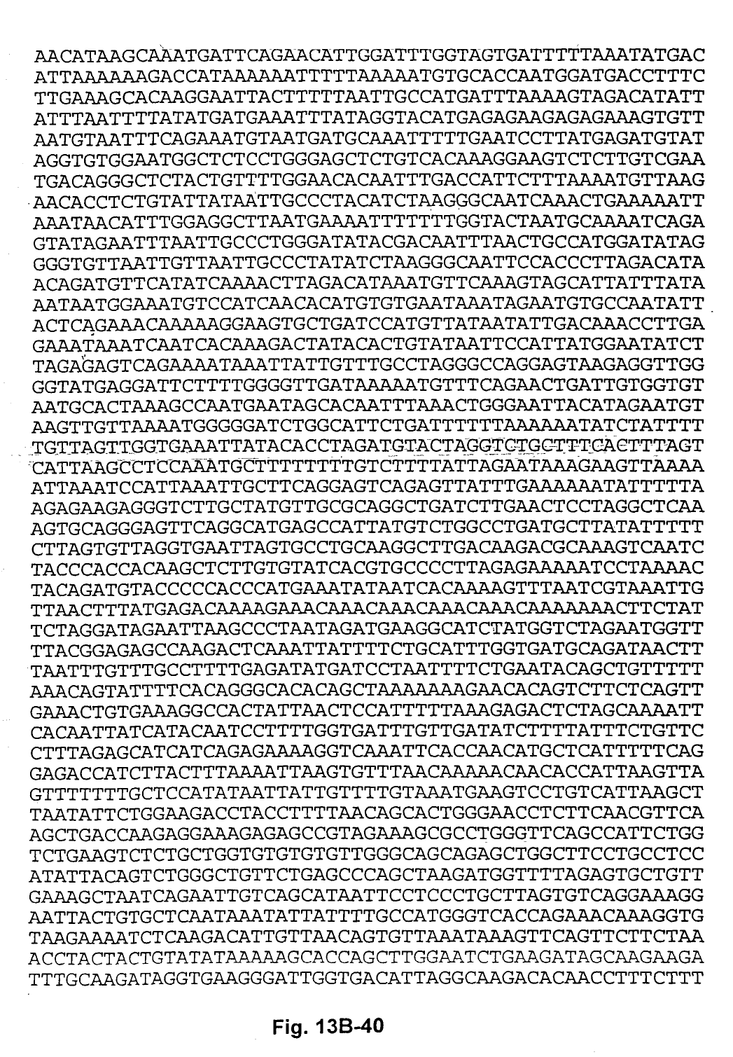

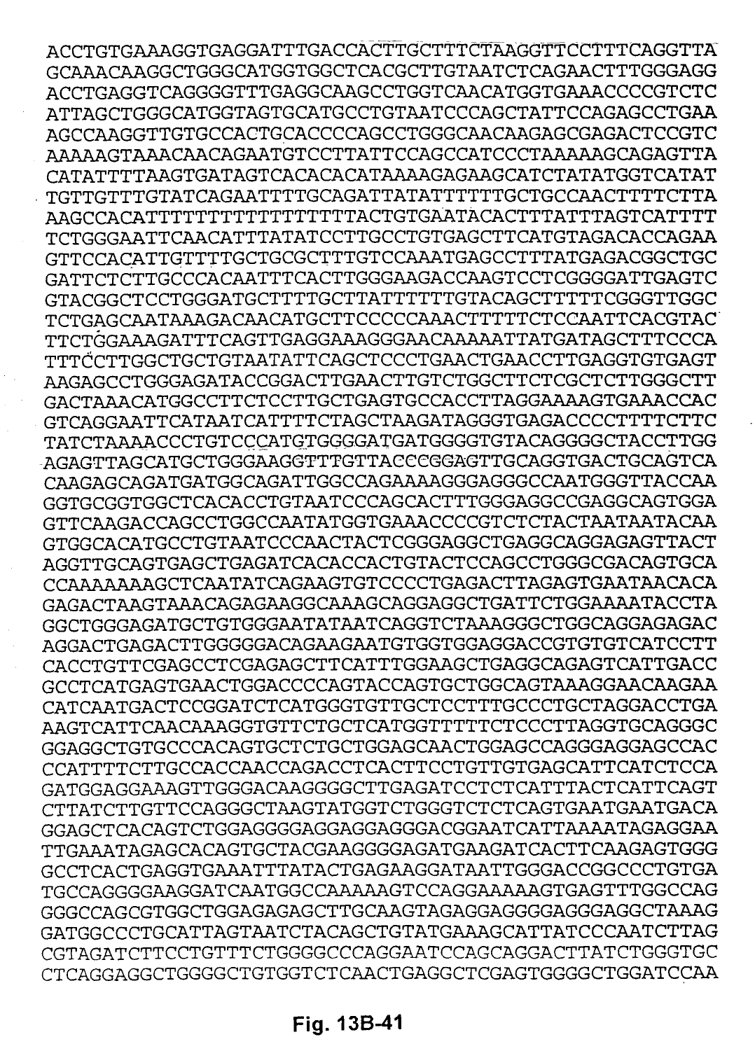

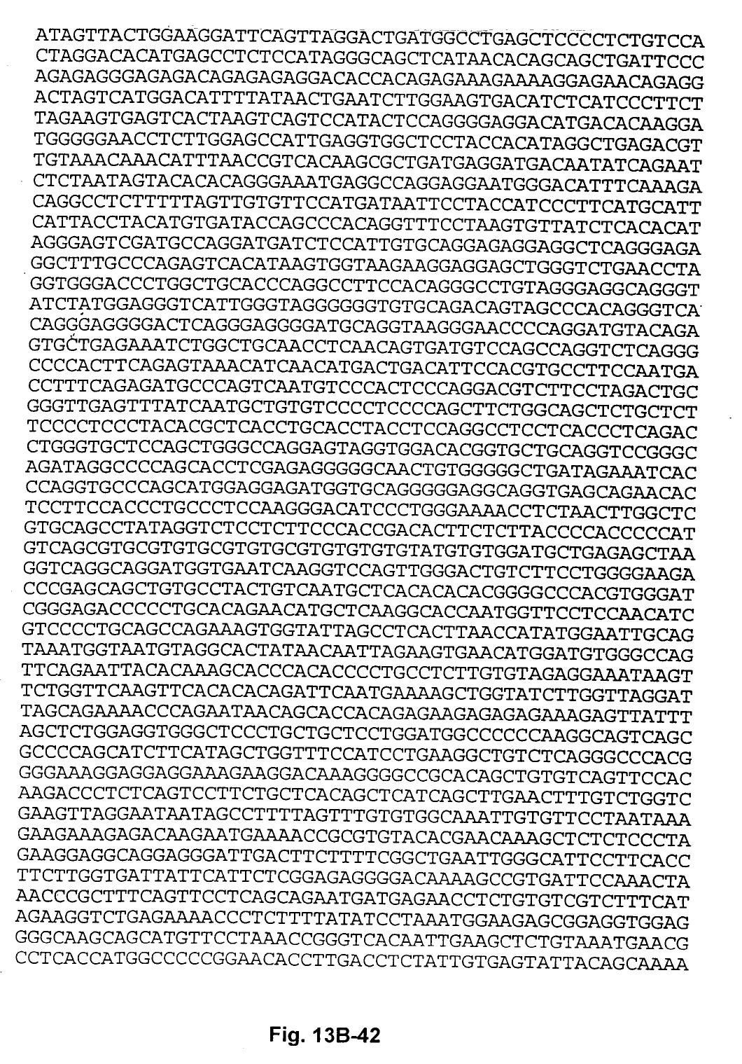

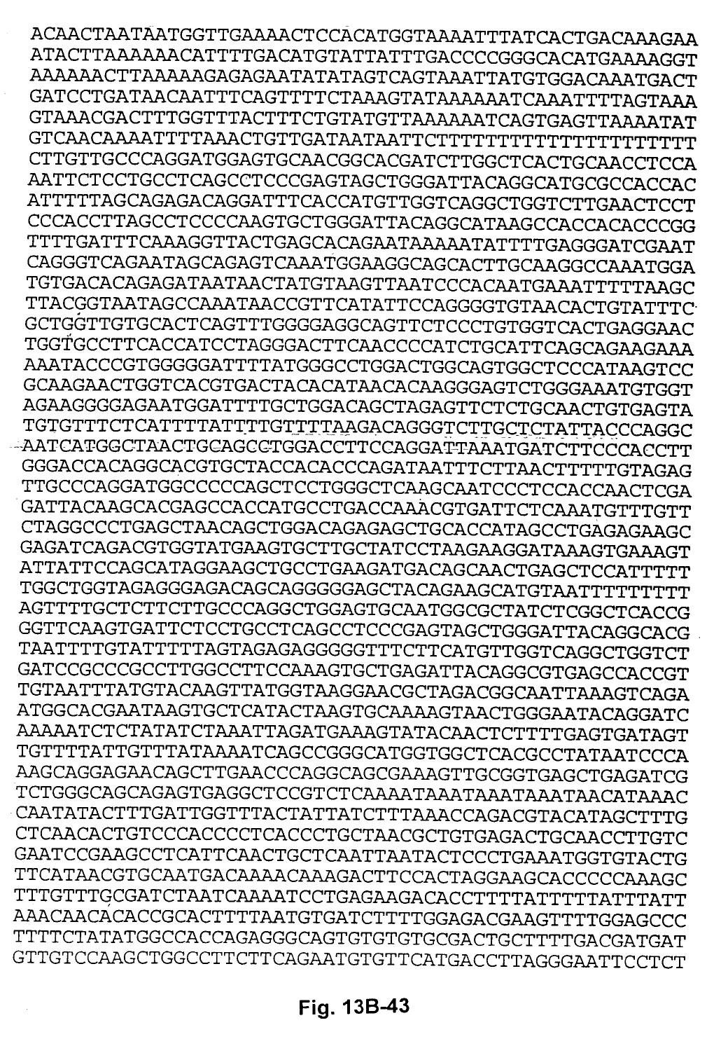

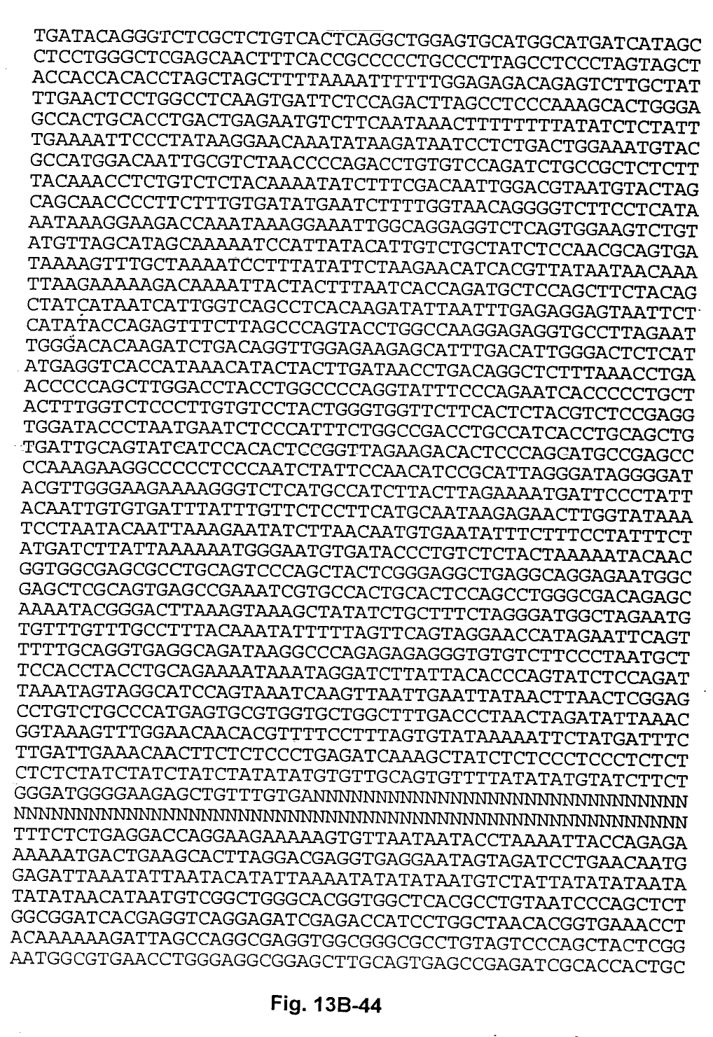

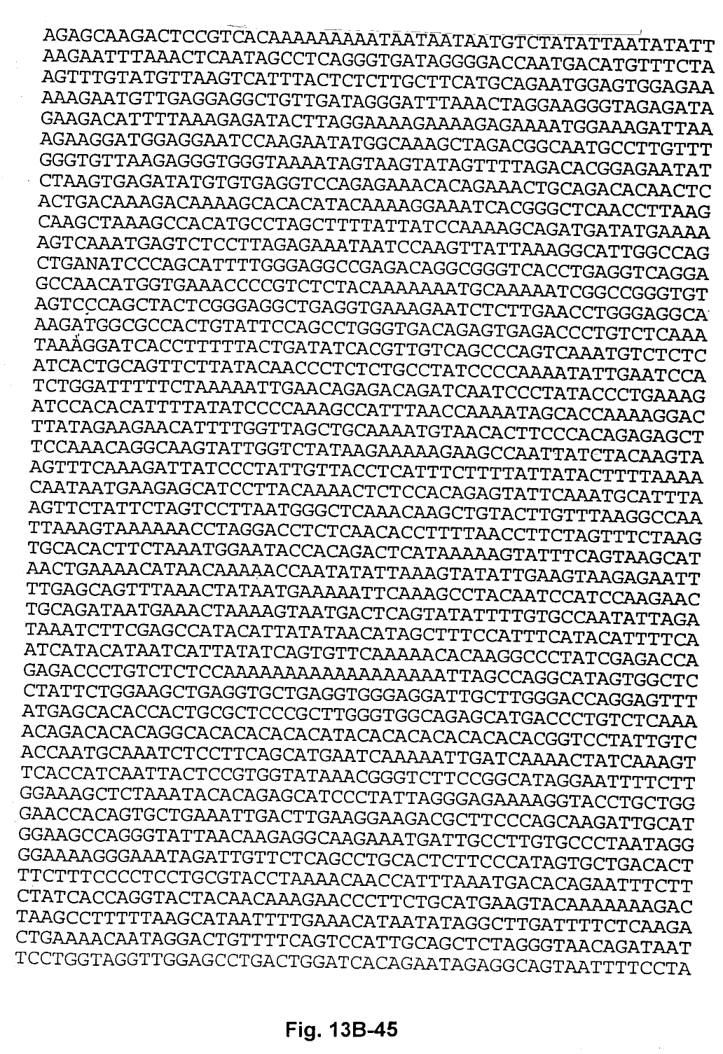

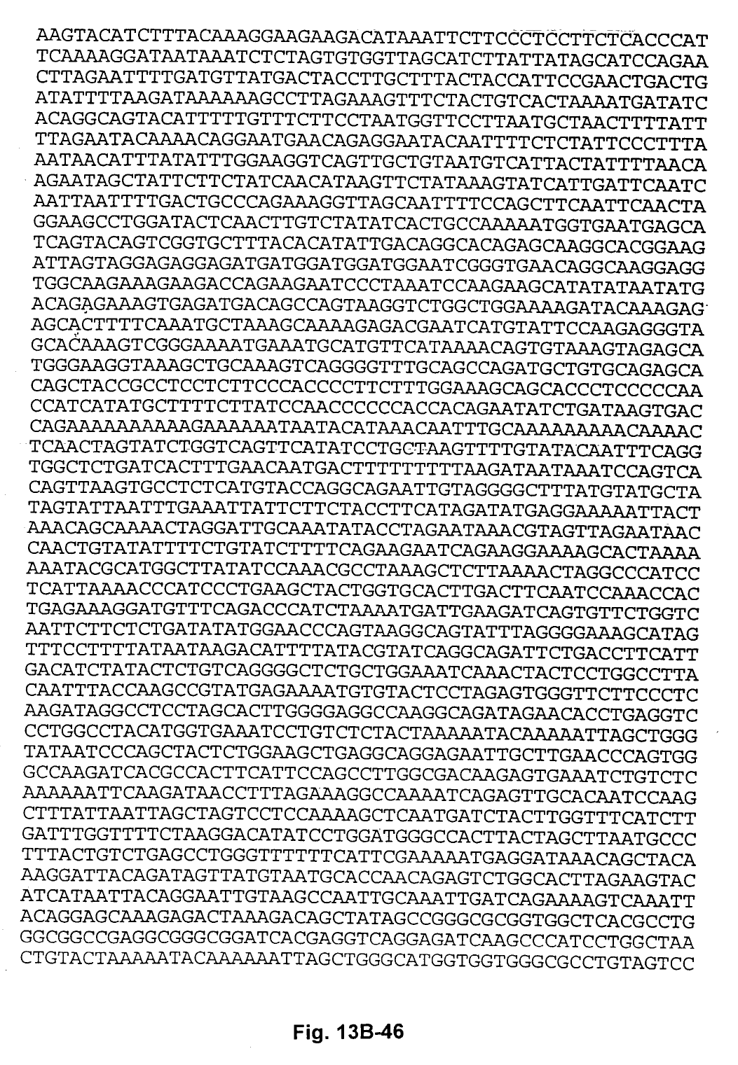

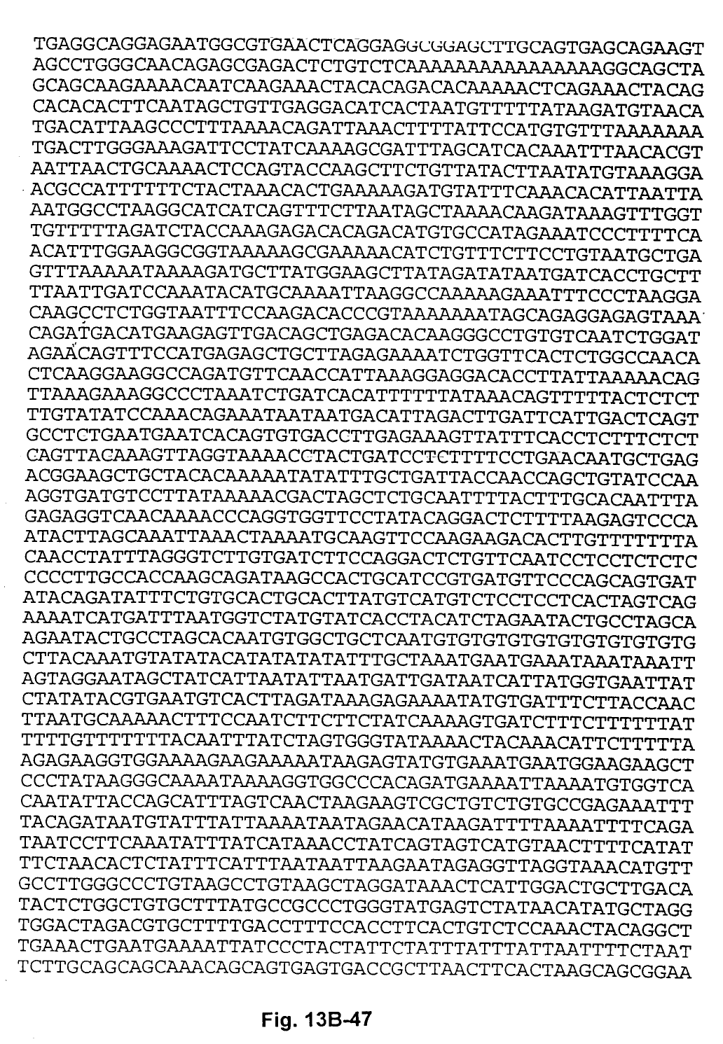

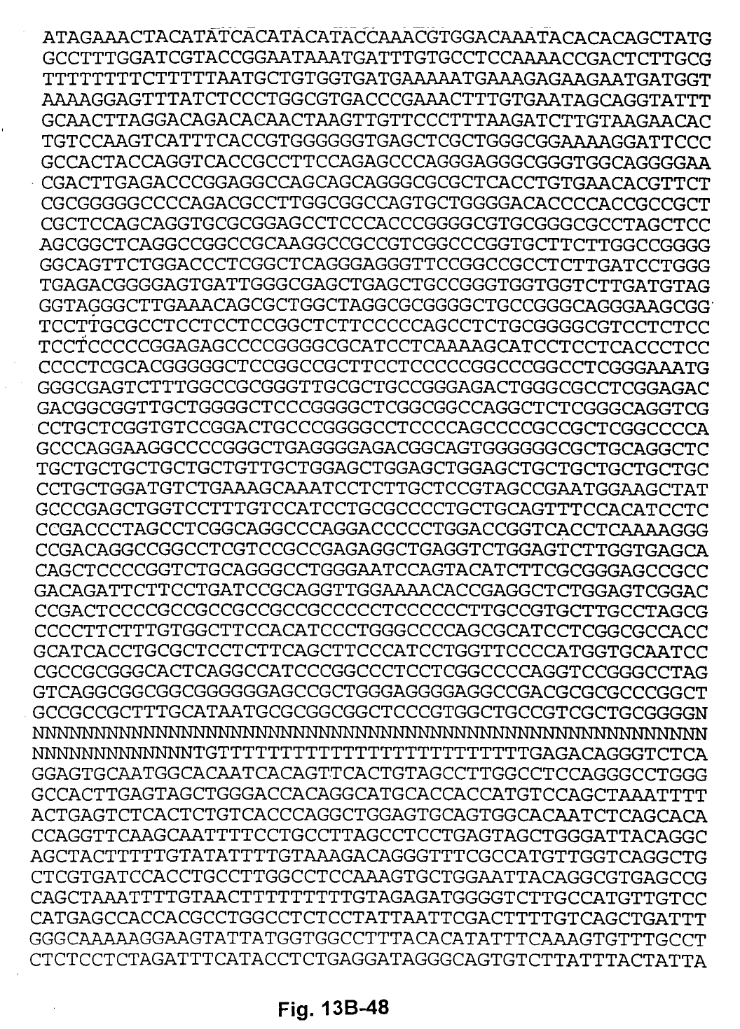

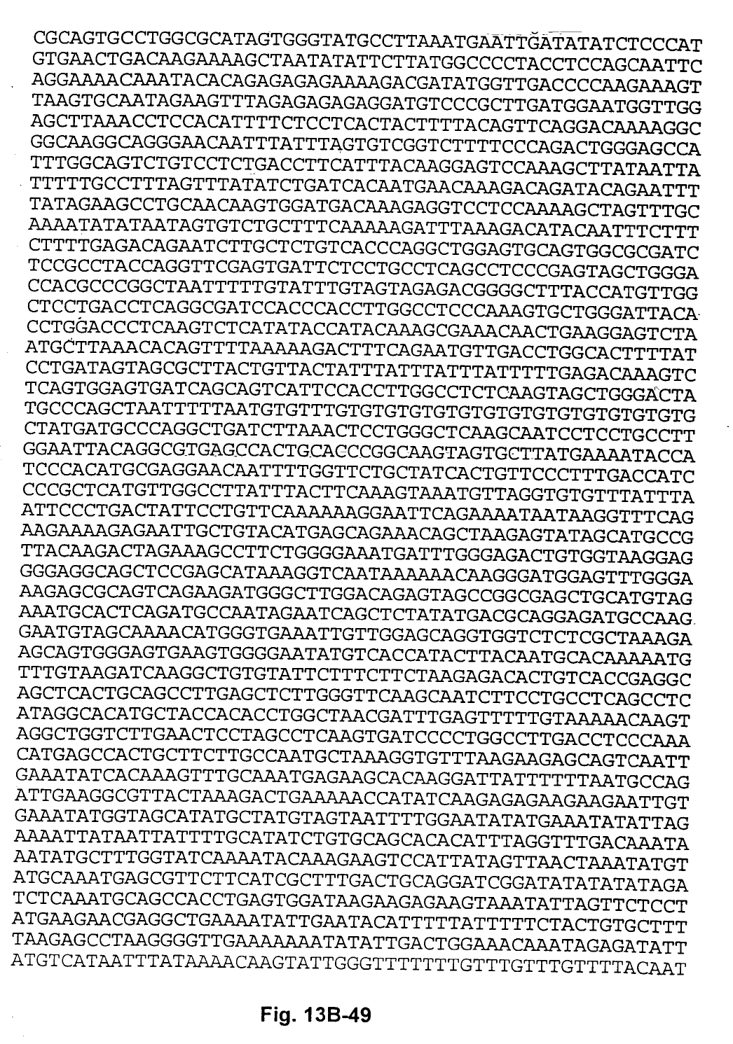

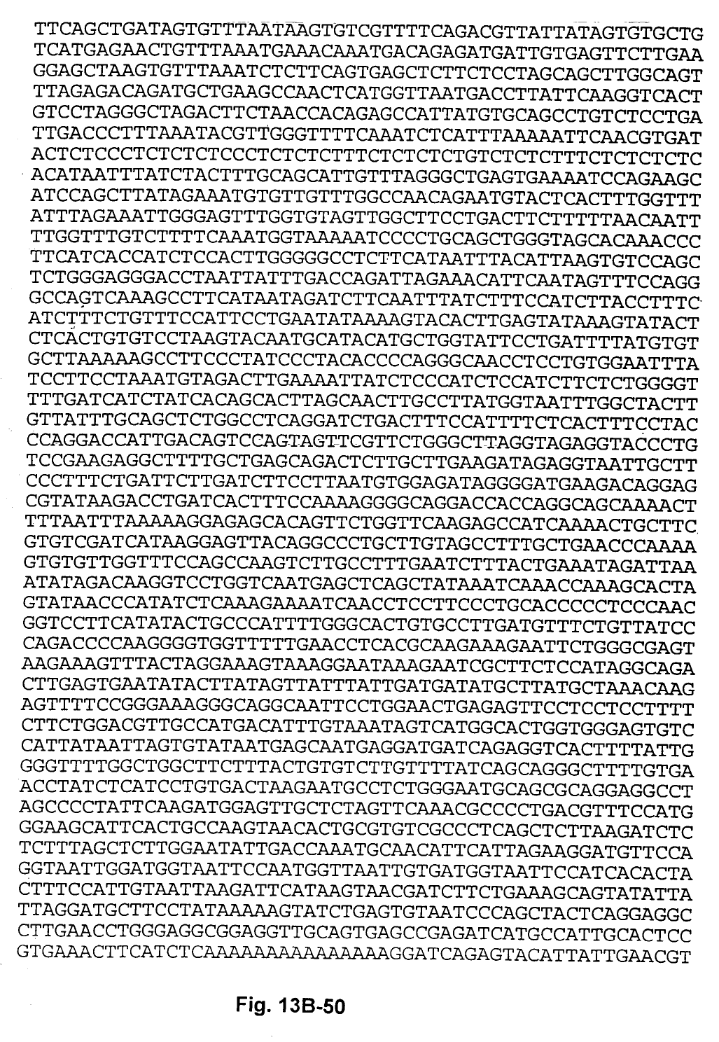

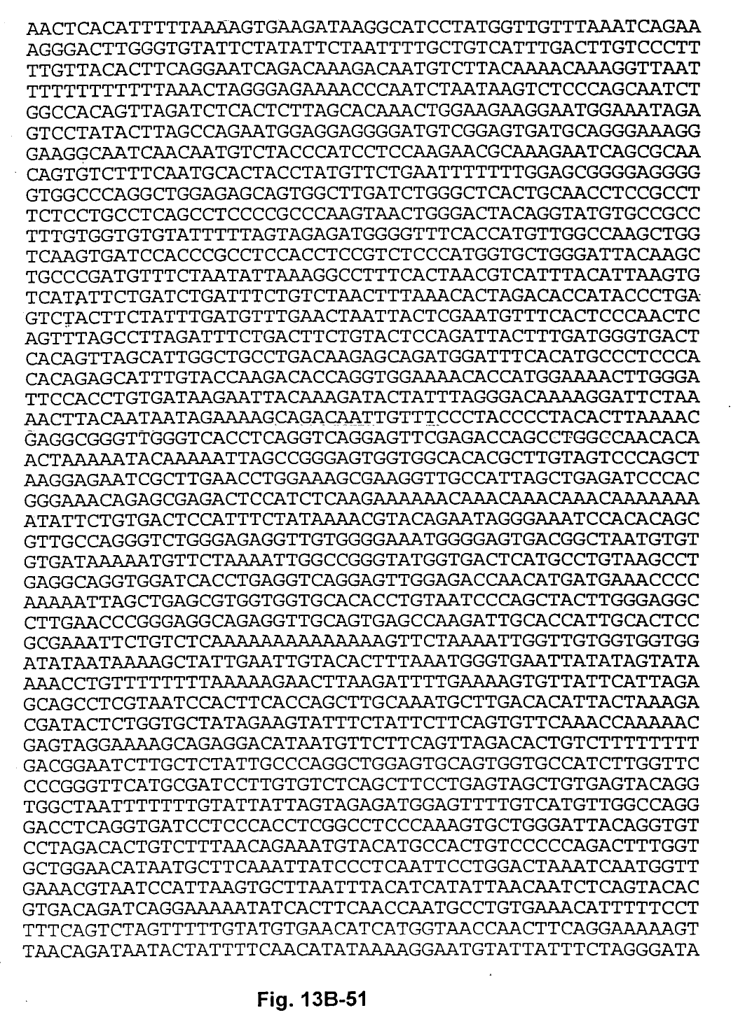

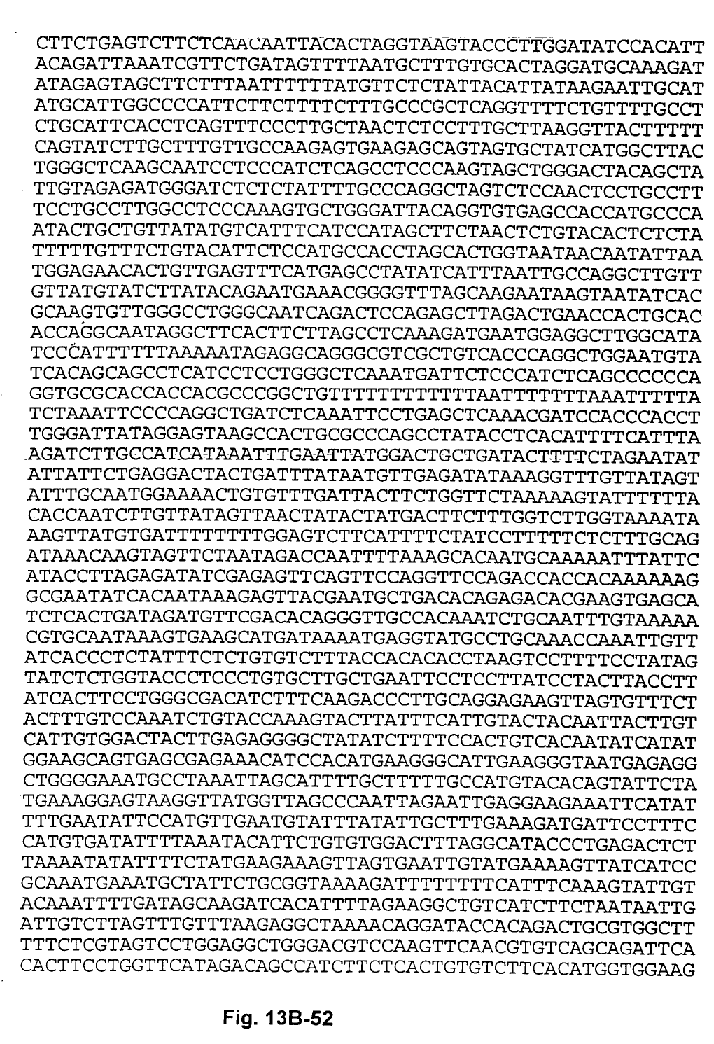

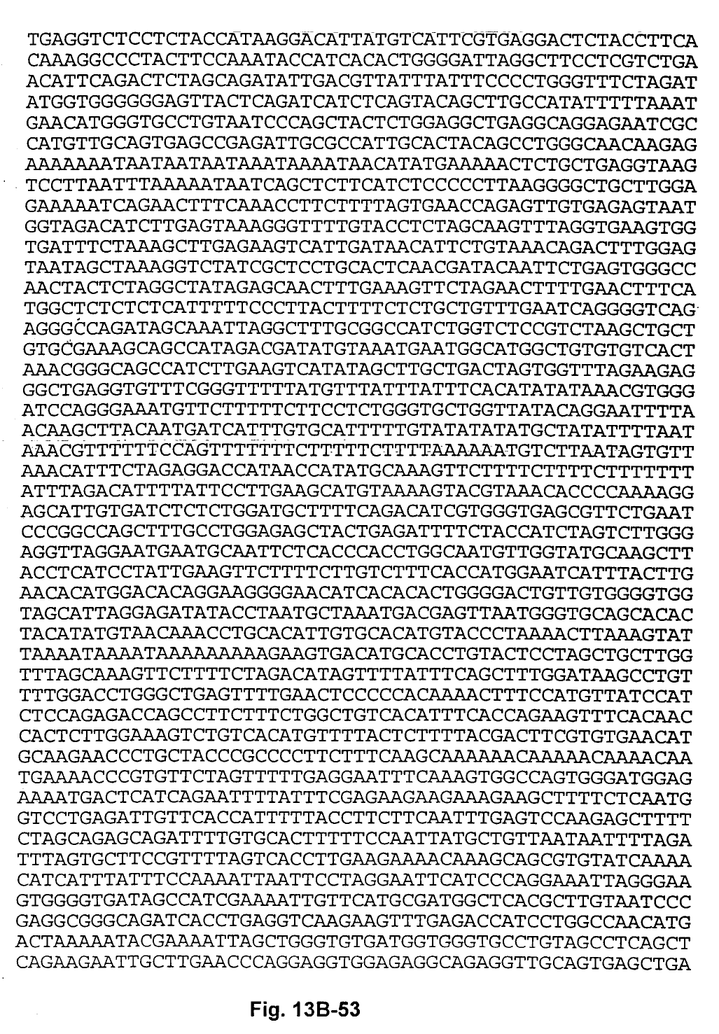

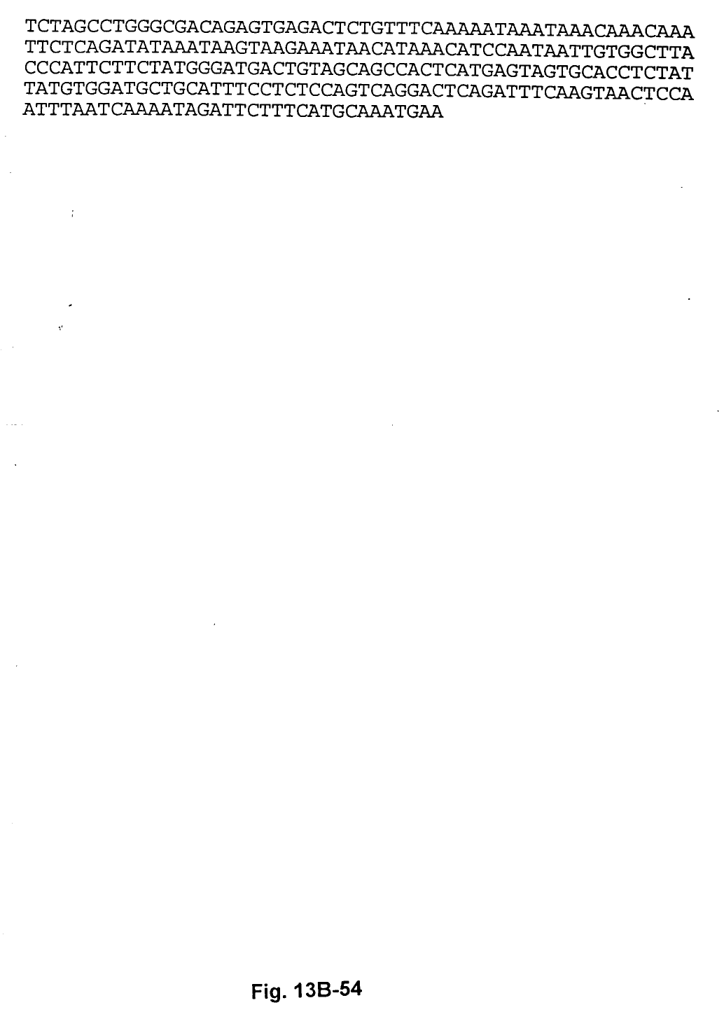

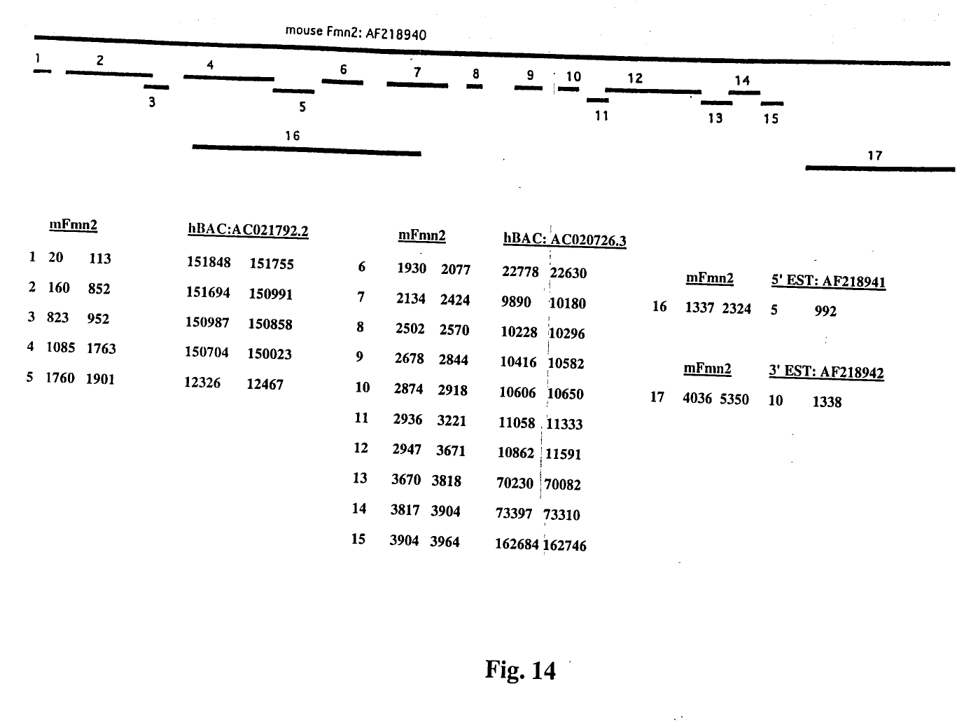

FIGS. 13A and 13B depict shotgun-sequenced human BACs containing Fmn-2 exonic sequence, as well as intronic sequence and promoter sequence. A schematic of the alignment is shown in FIG. 14. Determining exon and intron boundaries of human Fmn-2 using the mouse Fmn-2 cDNA (GenBank #: AF218940) and two BACs (GenBank #s: AC020726.3 and AC021792.2) containing human genomic sequence, can be performed as follows: Take mouse Fmn-2 cDNA sequence and one BAC sequence and perform a BLAST 2 sequences function (can be found on the web at http://www.ncbi.nlm.nih.gov/BLAST/). The alignments produced by this function will reveal the exonic human Fmn-2 sequence that corresponds with the highly similar sequence in the mouse Fmn-2 cDNA sequence. Using the nucleotide position numbers produced by the BLAST alignment, one can then go back to the BAC clone sequence and determine the exon intron boundary. This boundary can be found where the homology between the mouse cDNA sequence and the human genomic sequence.

Since the two BACs used at this time were “shotgun” sequenced, the nucleotide sequence consists of pieces of genomic sequence that are pasted together in a random order. At a later date, these pieces will be correctly ordered and the nucleotide numbering will be consistent throughout the BAC. However, at this time the exon intron boundaries can still be determined. The place at which the different pieces of sequence were pasted together is marked by a series of “Ns“(eg NNNNNNNN). Thus, the intronic sequence can be defined as the sequence from the “N” series to the first region of homology between human genomic sequence and mouse cDNA sequence detected by BLAST. For example: Determining the exon/intron sequence of Exon 7 (human BAC: AC020726.3-see schematic):

| | |
|---|---|
| Mouse Fmn-2 sequence: | 2134 to 2424 |
| Human AC020726.3 sequence: | 9890 to 10180 |

There is a stretch of“Ns” from 7010 to 7109 in the BAC that marks a site at which sequences were pasted together. The sequence from the last (3′ “N”) to the beginning of the next exon is intronic sequence. Thus, in the case of Exon 7, the 5′ intronic sequence contains 7110 through 9889. Then the exonic sequence is 9890 to 10180.

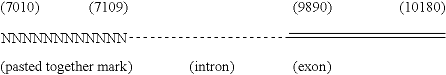

Materials and Methods

Cloning Fmn-2

In the search for additional mouse homologues of the Fmn-1 gene, human EST r56121 (3′ human EST, FIG. 1A) was identified in the GenBank EST database. This EST clone was sequenced and used in its entirety to probe a Clontech λgt10 cDNA library (catalogue #ML3067a) derived from 17-day-old mouse embryos (pooled from Swiss Webster/NIH embryos). Several overlapping clones were identified. A 511 bp probe from embryo clone 1 was generated using PCR (5′ primer: GGC TAG GAA GCA GCC GAT CGA GCC (SEQ ID NO: 12); 3′ primer: ACC CGC TCG GAG AAG TTG GG (SEQ ID NO: 13)) and used to screen a STRATAGENE mouse (ICR outbred strain) brain cDNA library (catalogue #936309). Two overlapping brain clones (brain 2 & 4e, FIG. 1A) were identified. Double stranded sequence of these two clones was obtained except for one stretch of approximately 200 bp of the repetitive proline rich sequence that could only be sequenced in one direction in both clones. Repeated screening of this library failed to identify any additional clones. A cDNA pool was then prepared from Swiss Webster mouse brain mRNA using Clontech's Marathon cDNA Amplification Kit (catalogue #K1802-1) and the following primer from Fmn-2 coding sequence: GTC TGC AGA GGC TGT CAA TCC (SEQ ID NO: 14). Rapid amplification of CDNA ends (5′ RACE) was then performed following instructions from Clontech's Marathon CDNA Amplification Kit. Four rounds of 5′ RACE were performed in total. Conventional PCR was then used to confirm the sequence obtained in the RACE reactions. Four clones (FIG. 1A) were sequenced from two independent PCR reactions which used Clontech Marathon mouse brain CDNA (pooled from BALB/c males) (catalogue #7450-1) as a template and the following primers: 5′ primer—TTC TGG AAA GAG GGA CGG CAG CC (SEQ ID NO: 15); 3′ primer—CAG CAT TTC TGG TCC CTG TAG ATT GC (SEQ ID NO: 16). The determination of the 5′ coding region of Fmn-2 allowed the identification and sequencing of another human EST clone (W39395) that bore homology to Fmn-2 in this region (FIG. 1A). The nucleotide sequence of mouse Fmn-2 including part of the 5′ UTR (224 bp) and 3′UTR (842 bp) has been assigned the GenBank accession number AF218940. The nucleotide sequence of the sequenced human EST clones r56121 and W39395 have been assigned the GenBank accession numbers AF218942 and AF218941 respectively.

Mapping Fmn-2 using SSCP

PCR was performed in a total volume of 50 μl of water containing 0.5 mM dNTPs, 5 μl of 10× amplification buffer (Boehringer Mannheim), 2.5 units of Taq DNA polymerase (Boehringer Mannheim), 25 ng of genomic DNA, 100 ng of each primer (5′ primer: GAG GTA AAA GAA ATC ATG GG (SEQ ID NO: 17); 3′ primer: CTT GAA AAT ATT AAG TGA AGC (SEQ ID NO: 18)), and 1 μl of [³²P] dCTP. PCR was performed in a Perkin-Elmer thermal cycler using 30 cycles of 94° C. for 1 min, 55° C. for 1 min, and 72° C. for 2 min. These primers match sequences in the 3′ UTR of the murine Fmn-2 gene and amplify an approximately 204 bp fragment which contains both size and sequence differences between the two mouse strains C57BL/6J and M. spretus. The (C57BL/6J×M. spretus) F1×C57BL/6J cross DNA panel (The Jackson Laboratory) was used for the reactions and products were analyzed on a 90 mM Tris-borate/2 mM EDTA/6% polyacrylamide gel. The gel was run at 4° C. at constant power of 40W, dried, and exposed for 1 night. Results were compiled and sent to the Jackson Laboratory Mapping Panels for linkage analysis.

RNA Blot Analysis

For Northern blot analysis, total RNA was isolated from different mouse tissues or whole embryos with RNA STAT-60 (Tel-Test), on the basis of the manufacturer's protocol. Poly(A) RNA was prepared from 250 mg per sample of total RNA (Boehringer Mannheim mRNA isolation kit (catalogue #1741985)) and electrophoresed in formaldehyde agarose. Membrane treatment, hybridization, and autoradiography with [³²P] dCTP labeled probe was performed according to the manufacturer's directions (Genescreen membranes, NEN Life Science Products). Two probes were used: Probe B (FIG. 1A) is described below in in situ hybrizations; the 520 bp Probe A (FIG. 1A) (northern data not shown) was generated from a PstI digest of a PCR product (5′ primer: GTG CCT GAG CAT CCT CCG TCC TCA GG (SEQ ID NO: 19); 3′ primer: GAA AGC CAT CAG CTG TCG CTG GAG CC (SEQ ID NO: 20)) using RACE clone 3 as a template. A Human 12-Lane Multi Tissue Northern Blot from Clontech (catalogue #7780-1) was probed according to the manufacturer's directions using the entire cDNA from human EST r56121 labeled with [³²P] dCTP.

For dot blot analysis, a Human RNA Master Blot from Clontech (cat#7770-1) was probed according to the manufacturer's directions using the entire cDNA from human EST r56121 labeled with [³²P] dCTP.

In situ Hybridizations

Whole mount and section in situ hybrizations were performed with digoxygenin-labeled probes using standard techniques. An 875 bp riboprobe template of the 3′ UTR of Fmn-2 (FIG. 1A) was generated using PCR (5′ primers—TTT TCC TCT GAA CCT CTT G (SEQ ID NO: 21); 3′ primer—AAC GAA TAC ATC ATC CTC AC (SEQ ID NO: 22)) with embryo clone 1 as a template. This was subcloned into the pBLUESCRIPT KS vector with T3 polymerase producing anti-sense and T7 polymerase producing sense transcripts. For embryo whole mounts and section in situ hybridizations, embryos were harvested from timed matings (E8.5, 9.0, 9.5, 10.0, 10.5, and 12.5) of wild type Swiss Webster mice (noon on the day after appearance of the vaginal plug was defined as 0.5 dpc). For both P2 and adult brains (age>6 weeks), wild type Swiss Webster mice were used. For section in situ hybrization, P2 brains were embedded in paraffin and sectioned at 10 μm; adult brains were embedded in O.C.T. (Tissue-Tek) and sectioned at 20 μm using a cryostat.

Gene Targeting and Animal Breeding

A mouse Fmn-2 CDNA probe was used to isolate genomic clones from a 129/Sv mouse genomic BAC library. An approximately 11 kilobase (kb) BamHI fragment of genomic DNA that hybridized to DNA probes of Fmn2 coding region 5′ and 3′ of the FH1 domain was subcloned into pBS-SK to create plasmid pBL9. A replacement targeting vector, pBL12, containing a pgk-neo cassette was designed to delete 433 amino acids of the proline-rich FH1 domain (FIG. 7A). pBL12 was created by inserting a 4.5 kb PvuI/NotI fragment from pBL9 into the PacI/NotI sites of the pOS.DUP/DEL vector (gift of O. Smithies). A 2.6 kb XbaI fragment was next subcloned into the XbaI site. TC-1 ES cells were electroporated with NotI-linearized pBL12 and selected in G418 and FIAU using standard techniques. Genomic DNA from 340 drug-resistant colonies was screened for homologous recombination at the Fmn-2 locus by Southern blot hybridization with an external probe (FIG. 7A) and three correctly targeted clones were identified. The targeted clones were verified by additional Southern blot analyses using alternative restriction enzyme digests. ES cells were injected and Fmn-2^{-/-} 129 and 129/Black Swiss mice were obtained. Genotyping was performed using Southern blot analysis (FIG. 7A) or PCR. Northern blot analysis was performed using a probe to the 3′ UTR of Fmn-2 (Probe B). After generating Fmn-2^{-/-} mice, we were notified that the Neo cassette in OS.DUP/DEL does not contain a polyA signal sequence (O. Smithies, pers. comm.). However, in the case of Fmn-2 targeting, this did not appear to compromise Neo function, as evidenced by the appearance of typical numbers of G418-resistant colonies, although very low levels of read-through Fmn-2 transcripts containing the Neo replacement can be observed (FIG. 7C).

Ovary Transplantation and Embyro Dissection and Analysis

Ovaries from donor 129 Fmn-2^{+/+} mice were transplanted into recipient, Fmn-2^{-/-} females, and ovaries from Fmn-2^{-/-} females were transplanted into recipient Fmn2^{+/+} females. Recipient females were mated to Fmn-2^{+/+} males and the offspring were genotyped. For embryo isolation and analysis, Fmn-2^{+/+} or Fmn-2^{-/-} female mice were naturally mated to Fmn-2^{+/+} males (noon of the day of the appearance of the vaginal plug was considered 0.5 dpc). Embryos were dissected using fine forceps at 8.5, 9.5, 16.5, 17.5, 18.5 and 19.5 dpc and visually examined for defects. Blastocysts were obtained by flushing with PBS the uteri of female mice sacrificed 3.5 dpc. Two-cell embryos were isolated by flushing with PBS the oviducts of female mice sacrificed 1.5 dpc.

Radiolabeled in situ Hybridization

Radiolabeled in situ hybridization was performed as follows. Frozen sections of ovary (11 μm) were mounted onto poly-L-lysine coated slides. The slides were placed on a slide warmer (37° C.) for one minute and then fixed in 4% formaldehyde in PBS for 15 minutes at 4° C. After prehybridization, sections were hybridized with ³⁵S-labeled antisense or sense Fmn-2 riboprobe for four hours at 45° C. (Probe B). After hybridization and washing, the slides were incubated with RNase A (20 μg/ml) at 37° C. for 20 minutes. RNase A-resistant hybrids were detected within 2-3 weeks of autoradiography using Kodak NTB-2 liquid emulsion. The slides were post-stained with hematoxylin and eosin and observed under a darkfield microscope. In situ hybridization experiments were repeated at least twice, using independent samples.

Evaluation of Meiotic Competence, Oocyte MRNA Injections, and Nocodozole Treatment

In vitro maturation of primary oocytes: in order to harvest fully grown primary oocytes at germinal vesicle (GV) stage, ovaries were collected from 20-25 day old wild-type and Fmn-2^{-/-} mice injected 44-48 hours earlier with 5 i.u. of equine chorionic gonadotropin (ECG). Ovaries were punctured thoroughly with fine forceps to release oocytes from follicles. GV-stage cumulus oocyte complexes (COCs) were collected with mouth-controlled pipette in maturation media (MEM with Earle's salts supplemented with 100 μg/ml pyruvic acid, 3 mg/ml polyvinylpyrolidone (PVP), and 10 mM HEPES). Oocytes were freed of attached cumulus cells and were cultured in the medium for 12-15 hours and the number of oocytes with the first polar body were scored. At the end of the culture, oocytes were processed for immunofluorescence microscopy.

In vivo maturation assay: oviducts from superovulated mice were flushed 14-18 hours post ECG and ovulated oocytes were scored just as in the in vitro maturation assay. In vivo fertilization was performed by mating wild-type males to superovulated Fmn-2^{+/+} or Fmn-2^{-/-} female mice. Fertilized eggs were flushed from females sacrificed 0.5 dpc and scored for male pronuclei and polar body formation. For mRNA injections, mRNA was prepared using a full-length Fmn-2 coding region cloned into a modified pCS2 vector (gift of Robert Davis), which was used for in vitro transcription using the mMessage mMachine kit (Ambion). mRNA was then purified using RNeasy Protect Mini Kit (Qiagen), eluted in water at ˜0.3 mg/ml, and injected into IMBX-arrested GV stage oocytes. After 9-12 hrs, oocytes were removed from IMBX by washing in maturation media, allowed to mature for another 12-15 hours, then processed for immunofluorescence. In order to accurately measure the ability of Meiosis I oocytes to proceed into Meiosis II, oocytes that died or remained in GV stage were excluded from the end result. Nocodazole treatment was performed by harvesting COCs, incubating them in maturation media for 6-8 hours, placing them in nocodazole (1 μg/ml in maturation media) for 2 hours, and then immediately processing them for immunofluorescence.

Immunofluorescence

In vivo/vitro-matured oocytes or fertilized eggs were fixed in PBS with 3.7% formaldehyde for 10 minutes at room temperature. Oocytes were then stained for one hour in dark at room temperature in PBS containing 1 unit/ml Alexa 594 Phalloidin (Molecular Probes), 5 ug/ml Hoescht (Sigma), and a 1:100 dilution of monoclonal anti-β tubulin FITC conjugate (clone DM1A, Sigma). Next, oocytes were washed for five minutes at room temp in PBS, mounted on slides with Prolong Antifade (Molecular Probes), and photographed using a Zeiss Axioplan 2 microscope and a 3CCD camera (Model DC336, MTI).

What is claimed is:

1. A method for determining whether a patient has an increased risk for recurrent pregnancy loss, said method comprising determining whether a formin-2 gene of said patient has a mutation, wherein a mutation indicates that said patient has an increased risk for recurrent pregnancy loss.

2. A method for determining whether a patient has an increased risk for recurrent pregnancy loss, said method comprising measuring formin-2 biological activity in said patient or in a cell from said patient, wherein decreased levels in said formin-2 biological activity, relative to normal levels, indicates that said patient has an increased risk for recurrent pregnancy loss.

3. A method for determining whether a patient has an increased risk for recurrent pregnancy loss, said method comprising measuring formin-2 expression in said patient or in a cell from said patient, wherein decreased levels in said formin-2 expression relative to normal levels, indicates that said patient has an increased risk for recurrent pregnancy loss.

4. The method of claim 3, wherein said formin-2 expression is determined by measuring levels of formin-2 polypeptide.

5. The method of claim 3, wherein said formin-2 expression is determined by measuring levels of formin-2 RNA.

6. A method for determining whether a person has an altered risk for recurrent pregnancy loss, comprising examining the person's formin-2 gene for polymorphisms, wherein the presence of a polymorphism associated with recurrent pregnancy loss indicates the person has an altered risk for recurrent pregnancy loss.


SEQUENCE LISTING (22 sequences)

SEQ ID NO: 1 - protein, 280 aa, Mus musculus
APPLPCTESSSFMPGLGMAIPPPPCLSDITVPALPSPTAPALQFSNLQGPEMLPAPPQPPPLPGLGVPPPPPAPPLPGMGIPPPPPLPGMGIPPPPPLPGMGIPPPPPLPGVGIPPPPPLPGVGIPPPPPLPGVGIPPPPPLPGVGIPPPPPLPGVGIPPPPPLPGVGIPPPPPLPGVGIPPPPPLPGMGIPPPPPLPGSGIPPPPALPGVAIPPPPPLPGMGVPPPAPPPPGAGIPPPPLLPGSGPPHSSQVGSSTLPAAPQGCGFLFPPLPTGLFGLG

SEQ ID NO: 2 - protein, 1567 aa, Mus musculus
MGNQDGKLKRSAGDASHEGGGAEDAAGPRDAETTKKASGSKKALGKHGKGGGGSGETSKKKSKSDSRASVFSNLRIRKNLTKGKGACDSREDVLDSQALPIGELDSAHSIVTKTPDLSLSAEETGLSDTECADPFEVIHPGASRPAEAGVGIQATAEDLETAAGAQDGQRTSSGSDTDIYSFHSATEQEDLLSDIQQAIRLQQQQQQKLLLQDSEEPAAPPTAISPQPGAFLGLDQFLLGPRSEAEKDTVQALPVRPDLPETTKSLVPEHPPSSGSHLTSETPGYATAPSAVTDSLSSPAFTFPEAGPGEGAAGVPVAGTGDTDEECEEDAFEDAPRGSPGEEWVPEMEEASQRLEKEPEEGMRESITSAVASLPGSPAPSPRCFKPYPLITPCYIKTTTRQLSSPNHSPSQSPNQSPRIKKRPDPSVSPSPRTALASAAAPAKKHRLEGGLTGGLSRSADWTEELGVRTPGAGGSVHLLGRGATADDSGGGSPVLAAKAPGAPATADGFQNVFTGRTLLEKLFSQQENGPPEEAEKFCSRIIAMGLLLPFSDCFREPCNQNAGSSSAPFDQDQLYTWAAVSQPTHSMDYSEGQFPRREPSMWPSSKLPEEEPSPKDVDTEPKSSILESPKKCSNGVQQEVFDVKSEGQATVIQQLEQTIEDLRTKIAELEKQYPALDLEGPRGLSGLENGLTASADVSLDALVLHGKVAQPPRTLEAKSIQTSPTEEGRILTLPPPKAPPEGLPGSPAAASGESALLTSPSGPQTKFCSEISLIVSPRRISVQLDAQQIQSASQLPPPPPLLGSDSQGQPSQPSLHTESETSHEHSVSSSFGNNCNVPPAPPLPCTESSSFMPGLGMAIPPPPCLSDITVPALPSPTAPALQFSNLQGPEMLPAPPQPPPLPGLGVPPPPPAPPLPGMGIPPPPPLPGMGIPPPPPLPGMGIPPPPPLPGVGIPPPPPLPGVGIPPPPPLPGVGIPPPPPLPGVGIPPPPPLPGVGIPPPPPLPGVGIPPPPPLPGVGIPPPPPLPGMGIPPPPPLPGSGIPPPPALPGVAIPPPPPLPGMGVPPPAPPPPGAGIPPPPLLPGSGPPHSSQVGSSTLPAAPQGCGFLFPPLPTGLFGLGMNQDRVARKQPIEPCRPMKPLYWTRIQLHSKRDSSPSLIWEKIEEPSIDCHEFEELFSKTAVKERKKPISDTISKTKAKQVVKLLSNKRSQAVGILMSSLHLDMKDIQHAVVNLDNSVVDLETLQALYENRAQSDELEKIEKHSRSSKDKENAKSLDKPEQFLYELSLIPNFSERVFCILFQSTFSESICSIRRKLELLQKLCETLKNGPGVMQVLGLVLAFGNYMNAGNKTRGQADGFGLDILPKLKDVKSSDNSRSLLSYIVSYYLRNFDEDAGKEQCVFPLAEPQELFQASQMKFEDFQKDLRKLKKDLKACEAEAGKVYQVSSAEHMQPFKENMEQFISQAKIDQESQEAALTETHKCFLETTAYYFMKPKLGEKEVSPNVFFSVWHEFSSDFKDAWKKENKLILQERVKEAEEVCRQKKGKSLYKVKPRHDSGIKAKISMKT

SEQ ID NO: 3 - DNA, 5762 bp, Mus musculus
gcccttttctggaaagagggacggcagccgggagagccaccgcgcattatgcaaagcggcagcagatgtgagcggggccagctgggcgctccttggcctccgctcccaacggccggcccagctgcctgaattgtcccggacactcccctggccctctccagggccgagatggcctgaggaccccaaggcgcggcggcgcagcagtggggttgaaacatggggaaccaggatgggaagctgaagagaagcgcaggtgatgcctcccacgaaggcggcggagccgaggatgccgcggggcccagggatgcggaaaccacaaagaaggcgagcgggagcaaaaaggcgcttggcaagcacggcaaggggggagggggcagcggggagaccagcaagaagaagagcaagtccgattctagagcctcggtgttttccaacctgcggatcagaaagaacctgaccaaggggaaaggtgcctgcgactcgcgggaggatgtgctggactcacaggccctgccgattggggagctggacagcgctcactctatagtcaccaagaccccggacctcagcctctccgctgaggagacaggcctatcggataccgagtgtgctgaccctttcgaggtgatccatcccggtgcttctaggcctgctgaggctggggtagggatccaggcgaccgcggaggatttggaaactgcggctggagcgcaagatggacaaaggaccagttctggttcagacacggacatctacagcttccactccgctacggagcaggaggatttgctctcagacatccagcaggcaattcgtctgcaacagcagcagcagcagaagctgctgctccaggactccgaggagcctgcagcgccccccactgccatctcccctcagcctggggcctttctgggcctggaccaattcttgctgggacctagaagcgaggctgaaaaggacacagtacaggcactaccggtgagacctgacttgcctgagaccaccaagtctctggtgcctgagcatcctccgtcctcaggaagccacttgacctccgagacaccaggttatgcgaccgccccctccgcagtcacagactctctctcatcaccagccttcacctttccggaggctgggccaggggagggagccgccggagttcccgtggctggaactggggacacagatgaggagtgcgaggaggatgcttttgaggatgccccccgcggctctccaggggaggaatgggtcccagagatggaagaagcctcacagaggctggagaaagagccggaggagggcatgcgagaatccattacctccgcagtcgcttctttgcctggaagccctgcgcccagcccacgctgcttcaaaccttacccactcatcaccccctgctacatcaagaccaccactcggcagctcagctctcctaatcactccccgtcccagtcccccaaccagagtcctaggatcaaaaagcggccggacccttccgtgagcccgagccccagaaccgccttggcctctgctgcagccccggcaaaaaagcaccggttggagggtggcctcacgggcggcctcagccgctcagccgactggaccgaggagctgggcgtccgtacgccaggggcaggaggctccgtgcacctgctggggcgcggggctactgcggatgacagtggtggtgggtcccctgtactggccgccaaggcacctggggctccagcgacagctgacggctttcagaacgtgttcacagggagaacactgctggagaagctgtttagccagcaggagaacgggcctccagaggaagcagaaaaattttgctcccggatcatcgccatgggtcttctacttcctttcagtgactgcttcagggaaccgtgtaatcagaacgctgggtccagttcagctccatttgatcaagatcagctttacacctgggctgcggttagtcaacccacgcattcgatggattacagcgaagggcagtttcccaggagagaaccatccatgtggccatcatccaagcttcctgaggaagagcccagccccaaggacgttgatacagaacctaaatcctctattttggaaagcccgaaaaaatgctcaaatggtgtccagcaggaagttttcgatgtgaagtctgaaggacaggcaactgtaattcagcaactggaacagaccatcgaggatctacgcacaaaaatagctgagctagagaagcaatatccagccctggacttggagggacccagaggcctctcaggacttgagaatggattgacagcctctgcagacgtcagtctagatgctcttgtattgcatgggaaggttgcacagcctccaaggactcttgaggcaaaatcaatacagacttccccaacagaagaaggtagaatcctgacattgccacctccaaaggcaccaccagagggtcttccggggtcccctgcagctgcaagtggagagtcagcccttctaacctcaccctctggacctcagacaaaattctgttcagagatttctctgattgtgtctccaaggagaatatcagtacagctggatgcccaacagatacagagtgcatcacaactaccaccacctcctcctctccttgggtctgatagtcagggacagccctcccaaccttctctgcatactgagtcagaaaccagccatgaacattctgtttcctcctcctttggaaacaactgtaatgtcccacccgcaccacctctgccttgtacagagtcctccagtttcatgcctggcctgggcatggcaattcccccacctccctgtctctctgacataacagtgcctgctctgcccagtcctacagccccagccctacaatttagcaatctacagggaccagaaatgctgccagcccctccccaacctcctcctcttcctgggctaggagtaccccctcctccccctgcccctcctctccctggaatgggaatacctcccccacctcctctccctggaatgggaatacctcccccacctcctctccctggaatgggaatacctcctccccctcctctacctggagtgggcataccccctcctcctcctcttcctggagtgggcataccccctcctcctcctcttcctggagtgggaataccccctcctcctcctcttcctggagtgggaatacctcctccccctcctttacctggagtgggaataccccctcctcctcctcttcctggagtgggcataccccctcctcctcctcttcctggagtgggaatacccccaccccctccacttccaggaatgggaataccccctccccctcctttacctggatcaggcataccccctcctcctgctctacctggagttgctattcctccaccacctcctctaccaggtatgggggttccacctcccgccccacctccccctggggcaggcatccccccacccccactgttgcctggctcaggtcctccacactcctcccaagttgggagtagcactttaccagcagcacctcaagggtgtggatttcttttccctccattgcccactggcttgtttggattagggatgaatcaggacagagtggctaggaagcagccgatcgagccttgccggccaatgaagcctctctattggacaagaattcaactccatagtaaaagagactccagcccttcgcttatttgggaaaagattgaagagccatccatagactgtcatgaatttgaagaattattttctaaaactgcagtaaaggagagaaagaaacctatttctgacacaatttcaaagacaaaggccaaacaagttgtcaagttgcttagcaacaaaaggtcacaagcagtaggaattctaatgtctagtctgcatttagatatgaaagacatacaacatgctgttgtgaacttggacaactctgtggtggacctggagacccttcaagctctctatgagaatagggcacagtcagatgaactggaaaaaattgaaaagcacagtcggtcttccaaagacaaggaaaacgctaagtctctcgacaaacctgaacagttcctgtatgagctgtcgctaatccccaacttctccgagcgggtcttctgtatcctgtttcagtccacattttcagagagcatttgctcaattcgtcgcaagttggaattgctacaaaaactgtgtgagaccttaaaaaatggaccaggggtcatgcaggtcctgggtttagttctagcctttgggaactacatgaatgctgggaacaagacccgaggacaggcagatggctttggactagacattttgcccaagctgaaggatgtcaaaagcagcgacaacagcagaagccttttgtcatatattgtttcatattatcttcgaaattttgatgaggatgctggcaaagagcagtgtgtcttcccactggcagaaccccaggagcttttccaggcctcacagatgaagtttgaagacttccagaaagacctcagaaaactaaagaaagacctgaaagcctgtgaggcggaagccggtaaagtgtaccaggtgtcctctgcggagcatatgcagcctttcaaggaaaacatggagcagtttatcagtcaagctaaaattgaccaagagtcacaggaggctgccctgacagagactcataaatgctttttggagaccacagcctactacttcatgaaaccaaaactcggcgagaaggaggtgtccccaaatgttttcttcagtgtctggcatgaattcagctctgactttaaagacgcttggaaaaaagagaacaaactgattctgcaagagagagtcaaagaagccgaggaggtgtgtaggcagaagaaaggaaaatcactctataaagtaaaaccgagacatgactctgggattaaagcgaagataagcatgaaaacgtgatctgtgaccagaacagcagaagaaaacaagacattgaaacaacttgcgcagcatccacctgacatgaaggagggaaggaagctccatcctcctagaccttttcctctgaacctcttgcataatcttcatgttttctaggcagtttgttgattgttgaaatttattgtatgtgcctttaaaatggcaaaagcagtagacctgtggagattggacacctttcctttttgtaaacattgtggtgttataccaacagacccaagtggcacgtctaagaggcactatctgcataaattctgaacatgatgagccctagcctatggtttctctcttgttgcaacaaaaatattttcctttttactgacaaccagaactccacatcgatgtggttagacttgtgggtaaacttcgttcctagtgaattctttgtatctacttgacttctgcttatacagtgtctatttgagaagtaaataccatcaatatgtcatgattcatacctctcccatgcttaagttgctctctatgatatttcccttgaagccctatgaaaaactggtctgcagcagaggtaaaagaaatcatgggatgtttctctatgtctaacgctgcaataaccacccccctctctctctcacacacacacatacacatacacacaattctctctcttacatacacacacacacatagtttctcactcatggaccgatacacatacacatacattctttctcacacacatacacaagcttcacttaatattttcaagaaatttgagtaagtaaagttcatgacaaatttattttttgacaaccacttattgtaaaaaaaaaaaaaaaaaa

SEQ ID NO: 4 - DNA, 1005 bp, Homo sapiens
tccctgcccggcagccccgcgcctagccagcgctgtttcaagccctacccgctcatcaccccctgctacatcaagaccaccacccggcagctcagctcgcccaatcactccccgtctcagtcccctaatcagagccccaggatcaagaggcggccggaaccctccctgagccgagggtccagaactgccctggcctccgtagccgccccggccaagaagcaccgggcagacggcggccttgcggccggcctgagccgctcggctgactggacggaggagctaggcgcccgcacgccccgggtgggaggctccgcgcacctgctggagcgcggggtggcgagtgacagcggcggtggggtgtccccagcactggccgccaaggcgtctggggcccccgcggctgcggatggcttccagaacgtgttcacagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatcaagatcaactttatacctgggctgcagttagtcaacccacacactcattggactattcagaagggcagtttcctaggcgagttccatccatggggccaccatccaaacctcccgatgaggaacacaggctcgaggatgctgaaacagaatctcaatctgctgtttcagaaactccccaaaaacgctcagatgctgtccagaaggaagttgttgacatgaagtctgagggacaggccactgtaattcagcagctggaacagactattgaggatctgagaaccaaaatagctgaactagagaggcagtatcctgccctggacacagaggtggccagtggtcatcaagggcttgagaatggagtgacagcctcaggcgatgtctgtctcgaagctctcaggcctcgtgcc

SEQ ID NO: 5 - DNA, 1952 bp, Homo sapiens
acgagggccaagtctctggacaaacctgaacagttcctttatgaactgtcactaatccccaacttttcagagcgagtcttttgcatcctgttccagtccacattttcagaaagcatttgctcaattcgtcgcaaactggaattactacagaaattgtgtgagacattaaaaaatggcccaggggttatgcaggttctaggtttggttcttgcctttggcaactacatgaatggaggaaataagactcgaggacaggcagatggctttggattagacattcttccaaaactgaaagatgtcaagagcagtgacaatagcagaagccttttgtcatatattgtttcgtattatctccgaaattttgatgaggatgctggaaaagaacagtgcctctttccactgccagaaccccaggacctttttcaggcctcacagatgaagtttgaagattttcaaaaagatctcagaaaactgaagaaagacttgaaagcctgtgaagttgaagcagggaaagtataccaggtctcctcaaaagagcatatgcagcctttcaaggaaaacatggaacaatttattattcaagccaaaattgaccaagaggcagaggaaaattcactgacagagactcataaatgctttttggagaccacggcatatttcttcatgaaaccaaaacttggagagaaggaggtgtccccaaatgctttcttcagtatctggcatgaattcagctctgactttaaagacttctggaagaaagagaacaaacttcttctacaagagagagtaaaagaagccgaagaggtgtgtagacagaagaaaggaaaatcactttataaaataaaaccaagacatgactctggaattaaagcaaagataagcatgaaaacttgaacaatgaaaagcagaatgaaaatgagtcattgcaacgactttcacaaaattcagctgacctgagagtgggagggaaactaccgtcattctgctcatgtttcttcttgacctcttgcataatctttttgttttctagacagttcactaattgttgaattttactgtatattcatataaaaatgcaaacgtactagaccagtggagaatttgacaccttttctttttgtaaaagtttatggtattataccgatagaccaaaacagcatgtgtaagaggcagtatctgcactaattctcaacatgctaaacattaactacaattcactgttgtgagaatattcctcgtcacagcaaaaacactttcctttctactgacaaccagtcctccacatcacagcatttagacatatgggtaaaatgttatttctagtgaattgtttgtatcagtttcatgtctaagtataaattttctattttaaaatttaagaaccgtttataatcagtgctttcccaactcttgggttgctctccataactatgtatttgtgaaagaaaatggtcattttttttactgaagtcatataatgacttgggtcagctcgtaatgcattgtgatggttttgtatgagctgggtgtttttttccattacttttaatgatcttcgttgcaagttatagttgtggataaaggggagaatttattgctcttgcaaaccaattatggaaagcaacttaagaaaaccaatgttctaaatcataattgtttgtatttatgtaaagtatggtctcttactttttagtttgtagtttaagtgcaaagaaacagtagtggttttttttctattgttttgtagtcttcctgtccccttcagtccctccagtgtgtatattaccattctccaatgaaataatagggcatttaacaaagatcgctatgtgcaatactgtatttagtgtttctatttcaatttttctaggatgttaatttatatgaaaataaaatgaataataaaagaataaagataaaaaaaaaaaaaaaaaattcctgcggccgcaagg

SEQ ID NO: 6 - DNA, 180216 bp, Homo sapiens
  misc_feature (1)...(180216): n = A,T,C or G
ccgaggcgggcagatcacaaggtcaggccaatacggagaaaccccgtctctactaaaaatacaaaaatgagccggcctggtggtgggcacctgcagacccagctcctcgggaggccgacgcaggagaatcgctctgaacccgggaggcggagcttgcagtgagccgagatcgcgccactgcactccagcctgggcgacagagcaagtctcaaaaaaaaaaaaaaaaaaaaaaaaggttaaatattccattttaaaaagttacctcaaaattccgagcataatctaaaattctggactccctatggactccaaatgtttcaggactgacctctttaccatcacaaaataagcctttcactcacgaaacaaaacatacttggatgagctgtgtatttacagagagaagacagtaagacaatgagcgggatatgcctctcagctaagaatggaaaataaacctccctttaaaatatacatacaaaatatgccttctaaaaatgaatgaagtagtctctaaacagacattttaagttaaaatgcagctatataaaattattatattatagcttctttaatggtatataccagtttgattcaaatccagtcaatttaactgaaacagaattcattaaacttcatttaaatagcatgacttatgattggctgcctaataaatgcaagatgtttttaattctgatataccatgtctttccacaaatgaagatgaggttaaatacctgaattttcctaaatgaggccaattccatgatctgcttatttgattatttaatagtactgaggctctgcctctcattgtcaacaaaatacagccacacctaggctctagttagcgcaggagaaaatcaaggccaattttatgaaaactatatgtaggcatgtgtgtgtgtatatatacatgtatgtatacacatacatgcatgcacatactatgtaatatataatattcaatatattatccacctccctacatcccttacggggggggggggnnnnnnnnnnnngnnnnncngnnncgnntcggngannaganatattatattatatatttacatgttgaaaatatgtaattatttttgggagacacagtcagtctattccagtacattttattccttgattaagtgaggggctcagagagatagaatactaccaagtctcccctggtcccttatagaatttctgcattatctcaaccgaacttatgttaaaacatttgaacaaaacacagaaatggaccaggcatggtggctcatgtctgtaatcccagcactttgggaggccaaggtgggaggatcacttaagcccagagttcaacaccagcctgagctacatgacaaaacctcatatctacaaaaaattagtaaaaatgtgactagcatgggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgaaaatgatatggtaatttgagatcacatatacaaaaacaaggagcactggtaaacattttttatctgcacaaaacatagtataattacatagttctctgaagtgatagtttgtggctgcagtgagctatgatcgtgccactgtactcctgcctgggtggtagaatgagaccttgtctctaaaaaagaaaaaagacaaaagtccgtgaacatctcaatagctgtagaatccgcagttaccaaatcgtatacactttagtgacacacacacacacacacacacacatacacacacacacacaaatgggactagaagggaacttcctcagcctgatagcatctgtgaaaaacccagggctcacattgaagactggatgtttttcaggaaaaagacaaggatgttcattcttgttacttctgtttagcaatgtgctagaagttctagtcaaggcaattaggcaagggaaagaaataaatggtctcctgattggaatggaagcagcaaaacattttgcagatgacatgatcttgtatgtaagaagtcctaaggaattcattagaagaaactattagacgtaacaaggaatctaattgtatttctagacattagcaattatcattgtgaaaatgtaattaggaaaacaactccctttgcagtattattaaaaagaataagatactgaagtatggattttaaaaaagaattttacactgaatctagaaagcattgttgaaagaagttaaggaggatctaaataaagggaacatgccatggttatggatgggaagacttgatattgttaagatgatgatacttgccaagttgtcctacagattcaacacagtgtctagcaacatcccaaccatttctccaaactcatttttttttagcaaaatagacaagctaatttaaaaattcacgtgaaaaatgaaggatctcagaatagccaaaacaatctagcaaaagaacaaagttggaagcctcctgatttcaaaagttattacaaaactatagtaattaagacaatatagtactggcatactaatacacatattggtcaatggaatatatttgagagtccagaaatcagttcttacatttatggtctgttgatttttgacaatggtactaagacatttcaagggtgaaagaatggtcttttcaacaaatggtgttgggacaactgtatatccacatgcaaaagaatgagattaggcaatggtttcttagatctatgactctaaaagcacatatgacaaaagaaaaaatagattattctacatcaaagttaacaattttgaatctacaaacaataccatcaaaaaacttaaaaagacaaccaacccacaaaatgggaggaaatgtttgcaagtcacaaatctgataagtggttaacatcctgaatatctaaataactcctacaactcaataacaacaataattttaaaaaaatctaatttcaaaatgggcaaggccgagcgcagtgactcacacctgtaatcccagcactttgggaggccgaggcgggtggatcacctgaggtcgggagtttgagaccagcctgaccaacatggagaaaccccatctctactaaaaatacaaaattagctaggtgtggtggcacatgcctataatcccagctactcgggagtctgaagcaggagaatcgcttgaacccaggaggcagaggttgtggtgagccgagactgtgccattgcactccagcctgggcaacaagagtgaaactctgtctcaaaaaaaaatccatcgcgtaagcttcgtctcgggccgnaatcgaacttacccaattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacaatagcaaagacttggaaccaacccaaatgcccatcaatgatagactagataaaaaaaaaatgtggcacatatacaccatggaatactatgcagccataaaaaaggatgagttcatgtcctttgcagggacatggatgaagctggaaaccatcattctcagcaaactaccacaggaacagaaaaccaagcaccacatgttctcacttataagtgggagctgaacaatgagaacacatggacacagagaggggaacatcacacactggggcctgtttggggttggggggctaggggagggatagcattaggagaaatacctaatgtaggtgacgggttgatgcatgcagcaaaacaccatggcacgtgtatacctatgtaacaaacctgcacgttctgcacatgtatcccagaacttaaagtataataaaagacaaaaaaaaaaaaaaacttaaaaaaaagaacaccctttcttgaccccaatttcccctaccagctataggtataccaccaggttcatgcactttgtctcagcaaaaagtctacacctgtagtctccaaatcactgtctttccattctctctcaagcaaaacaaagtctgacccggaattgctgatcttccgctcccaaacctgctctttctgcagttttccctatctcagtaaatgcaatgacaacctttcattaacttaggccaataatcctagagtcctatctttttctcacagtctacatcaccaaaacctgttgcctcctccctcgaaacatattaggaatctgatatctcacacgtctccactgctcccactctggtcctggccgccagcatctcacatctgttgctgtgatagtctcctcactgctcttcttgcctacctcccttcttcttgatattctgcactcagcagagcatcccttggaagacaaatcagaccaatcgttcctctacctaaaccctctgttagtatttcctctttcttagagtaaaagccaaacttcttaaaaagtcagcaaagccctacatgatctccctcccccgcagaccccattgtttatccaaatcacgtctccaattctctccccgctgagtgctctctccaggcactctcacatcgtgctgatccttcagtcaccccaggatccttgcagttgcctggttgttttccctcacatgctctttcctcagatatttacaaggttccttctgtcatggccttcagctatcaccttccaattgggctttccctgactcctccctctcctctcatgatgtgctccttatttccactctctctttttttttttttttcgtttcccccatggtacatattaaaattgtttttcttctatgggaacacagacatggatttttgtctctctgtctttatttctgtatcccactgccaggagtggagcttggcagagagtaggcaaacagttaatgttgagtgaatgctgaagcattggaagaaatcaaggaaggaaagaaagcagtaggagtgaacgaattatggcctccctgatgttaatgtcaatgctggcatctttctttgtggaaacaacagggactagtaagtacaacaataaaatcagtcactctcatacaccctaacattcatatgaaagtacttcactaaggcaatcatagtctttccatgcattctctttatctctctacttggcacagaagttggcatagagagatcccccaacacctgagagtcacctgaatcacagaactcacctgattcagagtcacctgaatcacagagagaagatacagcaagtggtaataatagaaggaataaagcaaattctcaagttatccatcacatctttttcctccctgtgtgtgtgtctctgccccctctgcctttcccctcatgccactcccatacacagaacatgaatgtgtactctagcgagacagtacaacactaaagcactgcacacagcaaccatagcctctaaccagtagactagctctatagctaagaaccacacaggaaaaaaacaaatcatgcactgtttttccagcttaagccactgatagaataagtaacattgagagttctcagcaagtacagcagaggttcagagcatggacctggacccagaatgcctgtgtttagatcatagccccaccacctgctaattaagtttcttttactactctatgcctcagttctttcatcctgaagatggagataaaaacacctgcctcatacagttgttgcaagtctttgcatagtaatgtctgtaagtgttccctattatgatcagctattgtctatcttaaatttggaaccataaaaatagcattaatcggaatcttgcagacaatctatttcaatgtcttcctcaattcagaaattctctttacaacttaattcataagaatttcatgctgtttttgacaatgatagagaaccctcctcttaagttccaccatttcctcaaatgagcctgctattatgctctagtggctaaagcaagggaacaattctttcctttataatctatcggagaagtgacaaagttcttacggagatttggaaactgtgctttttccattataggcctttctatacaacttattttcttaatcacgtgcatataattatctgatttaaattgtttaatgtaaaaatgtagtcagaggtatttggctgagaaaataaagatctcgctgtattcatgtttgaccgtgaaagaagatgcaagatagtcaacagcacgaaatgtcattgcttcagccatgccaaaagataacatgtcatcacatgactttgatgttttagctgatgtctcggcacactgagctgagaggaaggactatgattatatttgtagaatatacctcacaagaataggacacatgcggctccataagggaaaggtgaaaagtgtcaattattgcatcttctccagaactctcagaatcctcacttcatccaactggataaaggtggccagggagcatcctgagagtatccacttagggatgctggagaagtgcttgtcaaaacacaaagaactttcaatcagccaattttgaaatacaaacataactctacttatgaaaggggcttccccttgccccaaaataacagtgccaatccgacccctctcaaggattatgccaaaggaatctttttgcaaaaaccacaccttttacacaaagtagttcaagggtcaaagttgatgtgattaattatgggctcacactaagtgctctgtggtcaaacttgggctctacctacatgcatgctccatgtggtcctggacagtaccatacgcagtcacccactgatcttatagaacagatgcaacattatggggaggaaggccttgagctgcagtgggctgtcttcaggctcacagagtaaccctcctcgtgagggtcaagaggatggccaaatcaaacatacatctctacaaatacatgattctcaattcagacattaactacaatgagggaacagaataacctgtaagtcaagctcaagacttccaacaaccccaagtattgctgttctttcttcactctagaagctataatctaaaagtccacctacggaacaatgtcattaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngttaaaagtaatacagccaagtatgatttctaacgtacgcctcttccttccatatcacatttctcagtcatccgtgaaggaatggaaagagatttgccatgtccattccagttcagaaatattataatttccataattcatcatgtattttatactaaggattctagagatgccatctcagctccctgtgtgaggctctttcccataatatgttctattttaaaaagccctaggcaagttatttaccatttgccatctgttctaatattctttcttgaaatttccctttcaaacttaaaattgcttgtattttagttttgtgcaaaattatatctatataacacctcgatctctttccctatgggatcatgtgtagcagatgttaacatattataaactggaatgccattggttgttccaaaactaggatgtttcttaatttagaacatgacagcctggcgcggtggctcacacctgtaatcccagcactttgggaggccgaggtgggcgtatcacgatgtcaggagtttgagaccagcctggccaacgtgctgaaaccctgtctttactaaaaatacaaaaattagccgggcgtggtggcacatgcctgtagccccagctacgcaggaggctgaggcagtagaattgcatgaacccgcgaggcagaggttgcagtgagctcagatcacgctattgcactccagcctgggcgacaagagcgaaactccgtcgcaaaaaaaaaagtttacatttccttcatagagatggggggaaaaaggtgccctaaagatttttatcacatctatcatagcacccaaaaatgcctcatttgtagtaaataccctttctgtcttgtccatctgcattttttccactgcaaatgggttggatgtctgttcagatttaataaagaagatgtttgcttttcactttgtggagatatttcttggatccagagtttaaccttagcaagcagggtaagagtttagttttatacttaatattatctttatattacctgttaattattcttccacaatatgtaaaaatcaagtctggagtataagtgtcagcctcccaggtgggtaagaagcgtgtgcatgtggacctatggctgtttaccgtggcctatggggatcatctggaacactctggaacactctgtagtgttgcaggaaaccctggaacactgaagaaatagttctcaacacaagaagtctctcatcctctctttcccttttattccagcctttagtgagcaaatccacatctgtatgagcagtaattaaagtacgattgacactgcagtttccacaaaagtgggagttgtcttcctatgtttgtttgtcttcctttctctctatcaattccttcttcctctcttttcatctgttattcatattaaataatttacccttaagaaggcaatgctctttcctttttttttttttttttttttttgagacggagtctcgctctgtcacccaggctggagtgcagtggcgcaatctcagctcactgcaagctccgcctcccaggttcatgccattctcctgcctcagcctcctgagtagctgggactacaggcacccgccaccacgcccggctaattttttgtatttttagtagagacggggtttcactgtgttagccaggattgcctcgatctcctgacctcgtgatccacccgcctcggcctcccaaagtgctgggattacaggcatgagccaccacgcccggccaatgcccttctttctactcaaaacgttttttttttcttgaagggttcctggttgattgtgagttttaggaaaggaagacaccacttatatgtcagagtgccccatatgttggccagttgttatcagccatcttttttttttttttttttttttgagacagtgtctcactctgtttcccaggcgggagtgcagtgcagtggcctgtgatcatagattgctgcaacctcaaactccagggctcaagctgtccttccgcctcagcctcccaagtagctgggactacaggcgtgtgccaccgtgctcagctaatttttttaatttttaggagagatgcagtcttgctatgttgctcaggctggtcttgaactcctgatctcaagtgatcctcccacctcagcctcccaaagtgctgggattacaggtgtgaaccaccatgtctggccatcagccgtctttataaattgtggagaactgtcagttatcactttctggaataccctaggtaaaatattaaagttctagtaaacatgatataagcggtatggcacatgctcataatgcatttgtcttgcccatatggatatcaagaatattttatatattattattgggcactcattcagtgatcgcttttagagagcggtgnctccctttaaaattcgggattacctttanaatgttttcagggaacatcctaaaaaccagtgttcctaggctggaaagaaggctatcttagaaataaactggtttatcactcagagctttcttccctgttctcagtggtctatttgtcctcaatggtatcatttgaaaatgagacagatgtggacttaagattctgtggtaagtcacgtgtgttaaccactttgaaatctcagggtgcaagccaggggacacatagaaatattaataccatctttctgtcaaggagtccttctggaagtatgacaacaaacagatataattttgcttaattttttgctatttgcatttttccttatttcataactaactgtgtctgtccttgtcgcccttcacgaagttgttgacatgaagtctgagggacaggccactgtaattcagcagctggaacagactattgaggatctgagaaccaaaatagctgaactagagaggcagtatcctgccctggacacagaggtggccagtgtcatcaaagggcttgagaatggagtgacagcctcaggcgatgtctgtctcgaagctctcaggttagaagaaaaggaagtacggcatcataggattttagaggcgaaatcgatacagacttcccccacggaagagggcggggtgctgacactgcctcctgtggatgggctgccagggcgtcctccatgcccccctggggctgaaagtggacctcagacaaagttctgttcagagatttctttgattgtgtctccaaggcgaatatcagtccagctcgacagccatcagcccacacagagcatctcacagcctccaccacctccatcccttctgtggtctgctgggcaaggacagcctgggtcacagccgccccattctatttctaccgagtttcaaaccagccacgaacactctgtttcctctgcctttaaaaacagctgtaacatcccatctccaccacctctgccttgcacagagtcctccagctccatgcctggcctgggcatggtgcctcccccacctccccctctccctggcatgacagtgcctactctgcccagtacagccattccccaacctcctcctctgcagggtacagaaatgctgccaccccctccccctcctcttcccggagcgggcatacctcctccgccgcctctacccggagcaggcatactccctctgccccctctacccggagcgggaatacctcctccgccccctctacccggagcggcaataccccctccgccccctcttcccggggcaggcataccccttcctccccctcttcccggagcaggaatacctcctccaccccctctacccggagcgggcataccccctcctcccccacttcccggagcgggcataccccctccgcccccacttcccggagcgggcataccccctcctccccctcttcccggagcgggcatacctcctccaccccctctacccggagcgggcataccccctccgcccccacttcccggagcgggcataccccctcctccccctctacccggagcgggcataccccctcctccccctcttcccggagcgggcatacctcctccaccccctctacccagagtgggcataccccctccgcccccacttcccggagcgggcatacccccacctccccctctaccaggagcgggcatacccctccgccccctctacctggagtgggaatacctcctccgccccctctacctggagtgggaatacctcctccgccccctctacctggtgctgggattcccccacctcctcccttgccaggtatggggattccacctgctccagctcccccactccctccacctgggacaggaatcccaccgccccctctgcttcctgtatcaggccctccactcctcccacaagttgggagtagcactttaccaaccccacaggtgtgtggatttcttcctcctccattgccaagtggcttgtttggattagggatgaatcaggacaaagggagtaggaagcagcccatagagccttgtcgaccaatgaagcctctttactggaccaggattcaactacatagtaaaaggtaacatgaaagtgagctcgtctttgcacagtgtgtgtcagtattagggaagtgtttaccttcaaactcgggaaaattactgttgaatgtatttttaagcacaactctaaaactcgtctagatttttacatcaatatatagtgcagcagtttgctatttaaagctttaagcttttctccagaagtaccaaagatgtaaaataaatacatatacgtatcaatgtataagatgtaggtatacccatctatgtagttattgatatatctcttaaagattgagggatatatttttcttaaaatacatgctctgttatctagttgtaagactgctacttatttatgcctaaattccagtgaaagtatgcttatatgccctttggaaaatgaatgagggcaggtaagcagagacatttgtagcgaaattattttccatgtaagaatctactctttgcttttactacttcaagtttccatttctgacaaacagcttaaaaatgtaaatcaaattcttaaatccttttttttttttttggaggggggacagagcgagagtccgtctcaaaaataannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccctactataaagtgggaattaactcccctcttctggacaattttacacaactatcaccaataatatcagagggaaggaggcggaagaagcaaagttcaaaccaatagctaattccatctcttcaaatactaataaaaagatatgattataaaagatcttagaacaggtaacctgttaaagaaaacgtggcacatatacaccatggaatactatgcagccataaaaaagaataagttcatgtcatttgcagggatataggtgaaactggaaaccatcattctcagcaaactaacacaggaacagacaactgaacaccacatgttctcactcataagtgggagttgaacaatgagaacatatggatacaaggaggggaacatcacacaccaagacctgtcagggggtagggggcaaagggagggatagtattaggagaaacagctaatgtagatgacagattgatgggtgcaacaaaccaccatggcatgtatatacctatgtagcaaatctgcacattctgcacatgtaccctagaacttaaagtataataaaaaaataaaaataaataaacaaatttaaaaaagaatatgtatcctgtatgccaataccggttctctttttggtacctgctacattaggacaatttcttcccaaacaattatcagaataaggcttctcagctttggacccttttgagggaggacaatggcagaccaaaacagcagcaacatgacatgattccaatgagaagtcataattaacactgagatctcactggcatttctaattgaaatctaagagcctgtgcattgtccttgtaaggtacccttaactatataatttaaataaactgtttgggaaagcaatcattaggctcagaaattaaaacataaaaataactacaattttattgactgtatcaactgtacttctgatggaatgctaggtagtttacattattatctcattaaattttaagatatatgattttcataaatcatgtttaaacaggatagaaatttcagcaaggaggtaacatagcatttactgccaagggcccataattccagttagctttagacaggacatatataaaagacaggaaggaatcagagccattcataccctgggctgggtatccctgcagtatattgaaacgtgtttccgtaagtttaacgtgtcagactactaaatagatattaggtattccttgtgatgagatctgtaatctattttcaaatatttttgcagagggagtgtctaagagaccacacctcagggatggctgaccagcactacgcaagaaaggccattctccagaggcagttaacgcaagctgctcagctatctctatctaaatcaacaaccgaggctttaatacgcatactaatgtctgaaatgggctttcaatggtgaaattaaagtcattctggaggtgtggattagttgcaatctcttagagatgctccactcctacctcttccatagctagaataagagtctactgggggctaatagctttgttctttaaggaggctgcctgaggcctaggacctaaacagtactgggtgcagccactggctgagttgactttctagatgtctcgtgcagactatgagggtagcagattcctacatctgtgccaagcttatatctgtctgaagcaaacctttcagatagatcacagtgagagcccctataaagacagagaggtaaacaacctcaagtttatgaagcagggttttcaaactattttgcttgagatggccaggaaataccagtgtaatatatatctgtgtgtgggggagggttcagtctgaaacttgcttcagttcgatctggtagtagggtgacaatactactagcttatctgaaaagttcttggggatgggtgaggtggctcacgcctgtaatcccagcactttgggaggctgaggcgggtggatcacttgaggtcaggagtttgagactagcctggccaacatggtgaaaccctgtctctactaaaaacacaaattagctgggcgtggtggcaggtggctgtaatcccagctactcggggggctgagggataagaattgcttgaacctgggaggcggagattgtagtgagctgagattgcgccactgaactatgcctggatgacagagtgagactctgtctcaaaaaaaaaaaaaaaaaaaagtaaaagttttcactttacatttgttatccaggcaagattatcaatgacatccccttctactcttaaagcagcccagtgagtatttcgatcgaaccgtgacaatgttgggggataaagctgaagcttatggaagggcaaattcttttgcagctactgttatctatgaaaatggacctaagatctgtgaagagcacactgacccctgaaatacagaccaaacattgtataaaatatttaatcaattttcacagaagtgaactaccactatgaagctgtaggatactcttataatatcaatttatgggaataacaggccaaaattatttgaaatcaatccccccactaacaacaactaaaaatgctgagtaaaacatgtttgtaaaatatcataaggacgtcaaagagctgacaagatataattaccatgctaaacaaaatgaagagcgaactcagagaagtgatccagctcacaaactgcttttgccctgagtgtatttgcctatcctaacaaatgtcaactttctcttgacagtttcacggtgggaggggccaaatatcaaagcccagaacccactcatcatagagatcctagacataccccacaatgaaatgggactcaaaggactatactggttgtgcacatgtaccctagaacttaaagtataataataattaaaaaaaagggctatactggcaggggaaccaacccattcagtggacaaacagcactggctgaagggaccctcctacagcccccagagtgtgattcaaggtgttcctaaatccacagggcccccagaccctagcacacacacaaaaagggtaaatcttctctggaaggcactttcatctcagacttcaaattactataaaccacagggtgagattttttcagaggtattataactgtaggccagtttgaacattaccaaatagccagcagctcaggccttcattataacttttatatactaacacacacacaggtaattatttggggatgcccccttatatttgccgcattaataaacttaggaacctgacctcttctctcctctctgccttttgaatgtatgtgttgctgagaatgctccacatctattattgtgggaaataacctgtattcaaataacagactgagcaagtggtgctcagcaggaccagggttagaactttgatacctagaatccaaaccttacacactaattactctaatggagcactttgaaatataaacattacaaagtaaatgaaaacctagagtactgacagcaaagaaggagagaaaatgattcttgctgtttattacaatattacatatactttgttttagataaaccaatgctggtggtaaaaaaaaaaagcacaatcttggagaagcagcatgtctgaatgaaaaaaaaaaaagtactggttaatgaaggggcctgagtgcaagttcaatccatttcattaatgagctgcgtgatactgagagggtaactggaccactcacaggtcttattttgcctatcgataaaaatgccacatagccaaagaaagactaagcaaaaagaacagatctggaagcatcacattatctgacttcaaactatactataaggccatagtcaccaaaacagcatggtactggtataaaaatagacacacagaacaatgaaacagaacagacaacccagaaataaagtgaaatacttacagccaactgatctttgacaaagcaaacaaaaacataaagtgaggaagggacactttattcaacaaatgctgctgggataattggcaagacactgtaggagaatgaaactggatcctcatctctcacattatacaaaaatcaattcaagatgggtcaaggacttaaacctaagacctgaaactataaaaattctagaagataactttgggaaaacccttctagacattggcttaagcaaagacttcatgaccaagaacccaaagcaaatgctacgaaaacaaagataaatagctgggacttaattaaactaaagagcttccgcgtgtcaaaaggaatagtcagcagagtaaacagacaacccacagagtgggagaaagtcttcacaatctatacatctgacagaggaccaatatccagaatctacaaggaacacaaacaaattagcaagaagaaaacaaacaattccatcaaaaagtgggctaaggatataaatagacaattctcaaaagaagatatacaaatggccaacaaacatgaaaaaatgctcagcatcactaatgatcagggaaatgcaaatcaaaaccacaatgtgataccaccttactcctgcaagaatggccacattaaaaaaaaataaaaaaataatagatgttggcatggatgcagtgaaacgggaacatttctacactgctggtgggaatgtaaaccagtacaaccactatggaaaacagtgtggagattccttaaataactaaaagtagaactgccattggatccagcaatcccactactggatatctacccagaggaaaagaagccattgtacgaaaaagatacttgtacgtgcatgtttatagcagcacaattcacaattgcaaaaatatggaaccagcctaaatgcccatcaatcaacgagtagataaagaattgtggcatattccatatgatgaaataatactcagctattaaataggacgaatttaatggcattcacagcaacctagatggaaactggggactattattctaagtgaagtaactcaggaatggaaaaccaaaccattggtatgttctcacatttcataagtgagagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggtagggagcactacagctattccagagtatttaagccgggtttagtgagggatcctgtgggtgtttttgccaatactcccttgtccctataggaattttgctgatctctaacgttcgaggttatacagttgagtgtacactgtaatggaccgtgtatgtgggtcgtgtctgtaatgtcatctcttgggaggccaaggtggagatcacttaagcccagcgttcaacaccagcctgtgctacatgacaaatcctcatgtctacaaaaaattttaaaaattagctgggagtggtagctcacacctgtaatcccagcactttgggaggctgaggtggaaggatcacttgagctcgggtgacgaaggctacagtgagccgggatcatgccaccatacaccagcctgggtgacagagttaaaccctgtctcaaaaaaaaaaaaaaagaaaagaaaagaaatgagttgtattgacattgcacggcacagaatttcttctcttaggaaccattagagacacttctttgggatattagttgtgcttctgtgagtgactgtgttttcaatctcatggacactgaggaagacactcacattcccctttctgctttgcacctgagggctaaatttccaattacctttaaaaatggcagatccttatgagatacatttcacttggctgtcttatctcagcttcattttattgttccatcctaaattccttttcgtttgatctatattcctttatgatccttttgtacattttacatatttataagctgctcaaatctttttttggaacaaaggtgaattacaatgtattcagccagtgcacaactgcattttaaaggattttcacatgaaaatcttctgccatggttctttgtttctttcttttcttttattccttgcagtaggaagtcagctaccatatttctaccagatcctcttccatatattttcttctaggattttctgtgccttgattgcctttctaaaaagtcagttctctgtttcctctcagccattcacattttcaaagaaagggaagggatttaattttccacagaggaaacgacaatgatcaagtccctattcacatctacacaccttgctcctgctccaggttctccatgaatttgaaaactcaagttaattatttgatgttgttcaaatctattgaatgacctttgaaacttggtattgagcccctaaattggtccaagaactgttttagcccatacaaaactctccctcaaatcttgcattaatgtgctaagtagatgtttatttagatcttttccagcaaaagaatttcatcattcactcaaatatttaagtacatactatgtgccagaaagtcttgaaattctgaaagctgatggggaagacatgtatatgtataaacagaaaaattcaatacgtaaagcattcagatgacagagagatgaataggatgtcatgggactccagaaaagggtccatgagtgggaaacagccatggatgggtgggagactgctgtgtatagccaagggaaactgagggagaagataaagagaaaatgataaatagaaagggtttctttgtatcaatgtttttgccaccaactggtttgggttttttatttgtttaatgccaccaacatgggaagcacattacttccttttttcccagccagtcttatccttcaggggtgttctgcagacttcaaaaggtcaatgttaacactttaggcccatctgtggagacaatgccaactgcagagggagccaggatgagaacaaaggactgccaggaatctgcaactcactggtgagaagaggcaccaagacctcaccatggcctcagcccttccaattcatacaaagaagggtcagttccttcaccaatccataaaggaggaaaacaccatctttcacagaacagatcgtgaaaaagttaaagtgagatgctccctatgcagttaaagtgagatacatgaaaaaggcagaagacagagtaccacaaatctgaactagctggacatgtgaattgggtaaccaattttaagagctggaaaaaaggcccagtgcagtggctcacgcctgtaatcccagcactttgggaggccgaggggagtggatcacctgaggtcaggagttcaagaccagctgggccaacatggtgaaaccccgtttctacaaaaatacaaaaattagctgggcatgatggtgggtgcctgtaatcccatccctttgggaggctgaggagggaaaatcacttgaacccgggagacggtggttgcagtgagcagagatcatgccattgcactccagcctgggcgacagagtgagactccaaaaacaaacaaacaacaacaaaaaatgagctgaaaaaaacaacgaacgaagcacgcaggaaaaaaaaaagtcctcaattttctaagctagacctgcaagtctttcttcttcagaaaatgtcactgaactctttcactttgaattaaaatagaaaggggtcacccgggcacagtggctcatgcctgtaatcccagcactttgggaggcagaggcgggcagatcacttgaggtcaggaattcgagagcagcttagctaacatggtgaaaccctgtctctactaaaaatacaaaaattagcttggcatggtgttgggcgcatgtaatcccagctactccggaggctgaggcaggagaatcacttgaactcaggaggcggaggttgcagtgagccgagatggcgccattacactgtagcctgggtgacagagtaagaatccattaaaaaaaaaaaaaaaaaaaaggggtcatgtgttatatacagtggatttaaccaagatgtcactacagaagaatgtgcacctatcagttaactacaaatagatcattactattaataattatattatcaaactttgtgattgatgtaaaacatgagactgtaggcaaaggttacctatgatactgacatgcccactcttacaactgactgattagaagagagtgtggtatggttctgggaacgaggctgtctcatggaagtgtctctccaaatttacgtcagggagtcacaacagaagaagtttagaaagacccattagatcactctgatgaaaaaatgctatatgactgctggggtttatagatcaacaagagaaaaataagtcactaagttaaattacttcttacctccatcatcctcttcaagaatgcatttttaaaaaggagacaagttatttcattaaaaatcacttgctctagcattttgcaagctacatcaaatcatgaagcaaaaaccaggtgacaaacttcctggtatcaattccaactatcatcttttaccaaactaaaattcaggttatcacattcaattaacttcgagtcaagtgctccaccagtatatataccagcaacataaaatgtttcataatgttttataaagttccctaaaatttaagaatatcatataaatgtttcagttagaaaagttgagaatgatacagtagggaacagaaaacatgaaaatatcacccaatcaatgatgcaaaacatgggataaaagggctctccatgcaactgattaattcaactatatctacccctaagtattttgtatcaataaatgttatcataaatagagtagtgtagaaaatgggatcaatacttttaattgtgtgttttcctttgaaaacgaaaagtccttttacttgcttgattttcaggtgctatcatgttagaggtctctgatctctgaagatctttgcttaaaggaagtctgccttggcctttgagaaactgctctctgtaagtgttacgataaactcagtaacctggaacaacacttcctggaacaacacttcctacaccctctctctcatttgttgttcatttcagggagtacagcccacctagtgaatatcaaacagtgtttcaaaagcactggacatctcaaaagggagattcaaggtcaagtgtgggccctcatttcagtgcatcagtggaaagaaaaaaggcgcaaattcattcctccagagactgggatgcaaatgggaggcaagtgcagaactttgggtgggggactggaataaaaccttttacattgttggtcattgtcggttgaaagggacctcatgaataaggcaatgacatgaaacaatgataataattgtaataaataatattaaatacacggagctaggcactgcattctacattctaatgcttcatataaatgcctataattctcataacagccctacacagtacatgctatgctcatcaccattttacagatgagaaaactgaatacagttgttaggaaatttgcctaagattacaaagctaataagttggcagagcccaagctccatgcttttaagttatgctcttctactatgcatccaaaataagtttctaatgactgtcttcctctctatgagccaatgagaaaaaaaaaatcaaaattcttaactctaatatctatgttaccatttaggaaaatcgggtggaggagaaatctatgaatagcaatgatgttcttctatttactcctcactcatgcttcagaaatcaatatttatgcctggtcagtccaccaggaggaagttgggaatttattacaacaaaatgaagaaaagcccaggtgcggtggctcatgcctgtaattccagcactttaggagaccgaggtaggagaactgcttgagcccaggagtctggatcagcctaggcaacagagtgagactctggctctacaaaaaaattagctggacgtggtggtgtatggctggagtcccagctacttggtaggctcagttgggagaatcgcttgagccagggaagttgagactgcagcgagtcatgctccactgcactctagcctgggtgacagagaaagaccttgtccagaaaaaaaaaaaaaaagaagaagaagaagaaggggggaaaaaaggacaaaacaaccatattccataaagcattgattgttacacaaatatggttctcagttctgtatgacaaatataaagtgttttcttaaaaccaggcttggggtcatcatcctgctgaactcctccaaataaacctgaggctgatctcacgctatcttcccagccaataacacaaaataagacaacaaaaggaacaaagagagaaaagaccccaaagtaaaaactttttgaagattatatctgaatgccatgcaattaaaaataaaagattacttaaaactaagatttaataaaacttctctctctctatcttgcctcttccagttatctctgaagacaaaggaaagggttacctgtttcagcatcctcgagcctgtgttcctcatcgggaggtttggatggtggccccatggatggaactcgcctaggaaactgcccttctgaatagtccaatgagtgtgtgggttgactaactgcagcccaggctataaagttgatcttgccatatttaaaaataaaaaatgttgaaatgctattaattcagttacaaaaaaataagccatgactaatacattgcaaaatcatttctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnagaacggaagcaagctgtgatggatctaactaagaagaaagcaggaaccaaggagagggggtgggagagagattgaaacaacctactcagttgaggaacaacatccagatgatgttacaaccaggagacacagctctccaaactgaagtctaaaatatacatcaagacaaccaaatgagagagacaggctaggagagcacaagaggctcttcttggtaaccttaccaaattccacagctgtcagtctccttctgaggctgactcagacccaaaggcaggacaaacaaagcaagacgctttcacccacactatagcttgatcctctgccaagaggcacatctgggaccaagttcagaagcaggcttctagattcttaatcagtgagaggtgtcaaatgtcacctcaccaattagcaaactgtaattctgctttgcttctgagattgaatctaggctgcccgagtaaagaggattgtctggtgccaaccagatagtctggagataccacttgtgggcatggtcagtgtcagtgccttctgtggtctgcagtgccttggaatgctgttggatgcctgtgagatcagccaccctcccctgaggcttaccattaatctgagtaggagaatacggtagtgtaagtaagactgcagtcttccttggttgcgggagtagaccgaaaaaaaaaaccaaaaaacaaaaaaccctgataacctcttgctctgcaaaatctcatgcaagcattcttcacttttgagtgtagactagttaagatttaaaatgactgctgttttcatgaaatagtatcagacagttcagaaaaggctcatggttcctgattctcattgggtcaaaaatctagagatgaatttatctttccaaagtaagaaaactgaatccaaatcaaggttatgagatagccaacaacagtatgtctaaagagttcttggagaaatcacactttgtaaattcccaaggggcaggagttggccaatgtatgggttgttctccttcttaattttagatgtttatttcttatcgccttctattgtctctgctgttcctcttgttggaataaactcaagtgattttacaacatcataaatataactgccggttattggattacaaaataaaacactagcacctggcatttgcaagacattttatgctttgaaaatattttatactttaccgaatttgtttatctcaagttatgggaatattgcatatggattattaactgtattctacaggtaggaaaatttagatcttgaggccaaggagcttgtccaaagttacatggatgctaatttaggcaaagacatgtcctgaaatatggatctcctactctgatacagatcaaatcattggaaatcttgagtttgggaattgttctctttgcctttccttctattcttacatctcttgttcactttggatttactatttcatatttacttatattctcctggtcattagggtattcttgggtgcatttgatagctcttattccttgaatacattgcatacctctggatataccaccttgtgctctaatgtcgtttgagaaacaacagttttcacattctgaggttctgctttgaaatttaccttaattcatctctatcgttgagtaatcttatctttttctcttcagagaaagatttgccttattggatctggccatttatcccttatttaatgtccacttttcagcaaggtcatgacatgccatttcagaagaaagcaatatgctcttttaactaggaatcaggaaactaaaacttttgttgtggctctacatttagactgtgtgcaaattatttactagcttgtaaaaaattgtgagattttactagcttataagaaattgtaagattttactagcttgtaagaaattgcaagaaacttcaagttatctctattggatatttaatgtggagctctttgatgtcatcttaattagctaatattccatgtgacacatataaaatgcatactatgtttaaacttccgaattagcaataaacaccatgttgactgatcaaataattatttaaatgtggttttggctacttgtgtaataaatatagttcacataaactttctactatgtcttgatatgattctaagttacagaagatattatcttgcagttcttgaatttaatagtagaatcacagttcaaccattattcaaatacttccttttcaaaagaagtattcagtggttcccaaatctgcaccagcatcagctgtggtatttttttaaaacatacatattccagggccctactctagagaaaattgacttttcagaaggttgagcaggatcctttatttaagaaggtacctaagtaattctgatgtctagccaaggttggttgtctaaccatttttccaagtttattgtgggacatctaagctgggcatagcagtttaaacaaggtttacttattcagcggactttttggatgcttacaatatatcaatcattgtgctgagaacaacagtaagcaatatgacacagagtttgtctaaaggtattatactttagtgaaaattgcccccaaatcactttatcatgtatatgttgcagtaggagagtatatagtctcaaaagagtaaagaagtggctcctaagtcagctcctgtaagagttctgtgggaagtacagggatgttcaggagaagcatcacagtggaatgagacttgaacattcctgtccactgaatagtcagagtttgtcattttgaaaatagaaaatagaagagaaatacattatcaggggccactaaatatctgttaaaaggggactgacaggatgaaggcagtacagaatgctgggctgagagcaggactgagatcctagaggcagaatcccagtctacccgtactctctgggaggaagtgatgagaacttgcagcagggtggtggtagtgaaagagaagaatgaaagacaaggaagcatcagtggaacttgatgtgtggctggataccagaaatgcagaggaagttagtcaaaggttcccgtcctgatgactggagatcatggtatcactgactgagctctaggggttgtgaaatagagctcattaggcagaggatggtaagtgtctggtttttttatggttttccgtgcatttaccctactgcctagcacaatgcctgatgtgtagtaggggtattagttaaaatactggtttggtggtctctgttgtgacttgtaaaagatgaaaagttaattttatctttctcatggagaaatctaggcaggtaagtgcccagggaggtgcagtgcctgtgttccatcaggttgtccagagattcattccccaggccttctgttgcttcaccctcagggcatagtataactgatgatggctcacttccataattctgcctcagcctgagaggaaaaggatagagaaagtagagggccagccaggcacagtggcttacgcctgtaatccaggcacttggggaggccgtggcgggcggatcacgaggtcaggggatcgagaccatcctggctaacatggtgaaaccctgtctctactaaaaatacaaaaaattagccgggcatggtggcggatgcctgtagtcccagctactcgggagactgaggcaggagaatcacgtgaacccaggaggcggaggttgcagtgagctgagatcgcaccactgcactccagcctgggcgacagagcgagactccctctcaaaaaataaaaataaaaataaataaaaaaagtagagggccagccagccttttgaaggctgcgcttttatcgctctccgcattctgttgtccgtaacttagtcatatggctatccctggataaaagtaggatgtaggctggcgctgtggaacatggctgtaatcccagccacttaggaggctgaggtgggagggatcacttgagcctgggagttagaggctacaatcagctataaccatgccactaagagtacctcactgaatatccactgcagtctttggaatttgatactgttttaatcatttttctaggggaggtaactgaggctttaggatgttggggtagcttttctccaacacattagaaagcaagtaggagaacctcacaatctgtgcaccagaagaactggcatcacccgaaagctcgttagaaacaaagaacctcagtcccacccagatttagggatcagaatctgcattttaaaggatcccagatgattcaagtacacatgaagtttgagaaatactgctttattttacagagaccgtgttcataatctctacacagtcctgccttctagggaagcctgagatgataactttattaagagaattattaagagacgtgacattaaagcagattcaagataattcttttttctgattataagttctaattttatagatttcaaatgtgttaattctgcattttctgcgttttgcctcagttctgagatcatcttagaagagtgtactctcctcttcattgtattgtggtcactgaattctaagaatgttctaactttaggttcttcttctccaaggtgttggaaaaaaaggtcagatcatagcagtggttcgtaatgcaaagtttgggttacttaaggacactgtggaatccatccttttagacatctgtcctcttctgaatgtctgccttctgaaaggtggtagatgattattgttcctatgtaaaatcttaggcatttaaatgtaaaatccagtttgtgatcaagtgtgtttgaattataatagaaaaagcaggctgggagtggtggctcaagcttgtaatctcatcactgtgggaggcccaggcaggcagatcacctgaggttgggagtttgagaccagcctgaccaacatggagaaaccccatctctgctgtaaatacaaaattggctgggcatggtcccgcatgcctgtaacctcagctcctcaggaggctgaggcaggagaatcgcttgaacctgggaggcggagattgcagtgagctgagatcgtgccattgcactccagcctgggcaacaagagcaaaactccatttcaaaaaaaaaaaaaaagcaaattccatatatgaaatgtgaaagattctataagactttttaaaggactaacccttaaacatgagccaattgggtttaaaacctatgattaaaaagcaacatgcagatatcaacatttttagagaggtacttgggtatcagtttggagaaatggacagaaggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaattcattggtggttgtggccagagggaaggatattggtgtttaagacttcagaggtggaggtgttctactcattccaaagaccagtccatggtggagaggtaggctcgttgtgattagaattgagaagctcagggtgttggctgggacttaatgtcccatagaatgtagaagagtcaaccttgagaagcagagagaggaccatgtggtaaagtccttgacatgtgaaggctataccaaggaggtttgtcagggtctgtgatgaggaggggtagatgctgatacccctgggtgatacgggctttggaagtggagtgggttacgctcaggatggacgtggaatactctctagatgatcatggagggctagattagagggtcgcggtatgagggaggattggaaaaatcacctttcaggttcagtcatcgtttggcttggatgatcacattggccttttcattaccttcttgggtttgcctgttactggtttccagtccagtctcctcagccttgtctagtggttctcattccagggttatgttgccctgcagaggacatttgacaatgtccaaagacatttggggttctcacagtagggagaggggtgcaactagcttctagtgggtagagactggggcactgccaagcattctaaatgcacaggacatctccttctttcccctaacaaagaattacaaaatgtgcaaaatgtcagtagtgccaaggttgagaaatctagtgatatatgtctgatcatgttgttttattttattttttgagacagcatcttgctctgtcatccaggctggtgtgcagttgggtgatcacagctcactgcctgccttaacctcctgggctcaaccgttcctctcacctcagcctcccaaatagctgggactaaatgcaaatgccaccatgaccagctaattaaaaaaaatttttttaatagaggtggggtctccttatgtggccaaggctggtcttgatctcctgggctcaagtgagcctactgccttggcctcccaaagtgacgggattacaggcatgaaacaccgcacctggccatgtcattttaattgtttctctatcagaataaactccagcttctttggatctccatggtcttccttctgcctttcaatactttcccacttctacctctcctgacccactttcctttccaaactcttttgccacgctgaagtgttattgattttcctaattgtccatgtcttctctcacttgtggggctctgtgggcacctctacttctgctccccactattatttcccagttagcctccactctttctcataggatgctattcagagttagatgccattccttcccagacatcttctttagccatctcagtctgcattttctctctcaaaggatatattacacatattataattgcctatttaccctgtaacacctgaatgcaaattaaatattgtgtgaaggttccttgaacaatgtaatgctgtgtccttgtgtttacctgacagatctccccagagtagttaggagtggtctctagtcctagactggattctaatcccatcttctccacccacttaagaggtgaaaacatttacccaaggtcacattaaaatttttcatgtctaataatctctggagttgttttgaagattaaatgagattaatttattaggcatttagcacaatgtctagcacatcatcaatgctaggtaattattagtgattggtatttaatattattgcctttcaagttcttattacagaatcaggcataactgtttaataaatatgccttgattttgtaaagaagagagctgtaggaaagccatgatgactgcagtgaggaggaactggagaatctattcagtgaattgtcttgaatgatcaaaggttgcctatgtttgataatcttgaaaacacttggactgggtgtggtggctcacacctgtaatcccagcactttgggattgggaagctgaggcaggaggatcgcttgagcccaggagtatgagaccagcctgggccatatagggagactctgtctttacaaaaaatttaaaaaattagccaggtgtggtggtgcacacctgtagtcccagctattggagaggtgggaggatcacttgagcctgggaggttgaggttgcaatgagctgtgattgcaccactgcacttccacctgggcaatagagtgagaccctgtcccaccaaaaaacaaacaaaaacaacaaataaagagagacagagagaaaaaagaaaacacttgaagagggagaagatgggagaagggatgggatctggctgtgttctggaattaatttattagtctgggagttgactctgtattggatctcttcttctgggcttattgccatcctcctttctcaatccattttacttaattttactgctataattaccacattccttcaccaaccctcatgcccccacatctttctggatggagtggaagggaaatggaaagagtagggaaagctacggggaggtaactgcaaattgtgaagggaaatctgtcatgcccacttggtgttatgttcatgaccatcccgtagtgactccatgctttccctaggtcagtctgtggaagtattgcacttgaatattgacatgcgttgtgcttatttagctctctgtgtgtatgtgtgcaagtgtctggcatacttgacagataacgtggcatggaagaatgaatcgttttgctttccctagcacctcaccaaagaatagggacctccatttcatctagaattcctaagacagggaggaaccagagagggctggagattgttttgctacacaacattgctcttctctttcaaagagttcattttattttcttgattggataatttcttgtggggtaaaatcatggatgggaacagtcaatacatataaataggaagaaatctgcttactgagttttctgtaagggatattttccaatccaatattacttttttcttatgaagtcaatttatttcataaagaatataataagtggatatgtatccagaagttcctactcgatttctttctttgaaggctatgcacttgttatatggtttgaaaaaaattgcctcgtatggggaacttttctttaccacaatattgattgtatagtgagttaaattatgtcccctctgtttgtagtcagatagttcagtgtgaagtagaaggctttcaatatatattttaaaaattgttaattctctggaatctatcattttcgaaaggactttgtcatgtttcttttttttttttttttgagacagagtctcactctgtcacccagttggagtgcagtggtgcgatctcggctcactgcagcctccgcctcctgggttcaagcgattctcgtgtctcagccttccgagtagctgggactacaggtacatgccaccatgcacagctaatttttgtatttttagtagagatggggttttaccatgttggccagagggccttgaactcctgaccttaggtgatctgcccaccccggcctcccaaagtgctgggattacaggcatgagccaccatgcctagtcatgtttttttttttttaatcaatttatagtaatttgtccccagttactactcagtgaatgtttgtatgactggatcttgtagtaatcagtaaccaagtttttatacatatgtgtttaagcatatgtatttttaagcatagttggagattattatgtttaagcctattttgagattattttatttttctgatccggtacgtgatttaataaatttctaatatgtgttgccatttatgtatcttatttcagagggcaaaacgggtgatgtttttcccccgttatgctcaattcaagtgcatcgttgcaaaagtagtaatgatagttattgttattcccccgatcatgaagttttctgagctcagaggaagtgctaccagtcagtagtttcatagattttagaatgcttcccaacagtaaagttaattgaagcttagaaaaatagatttttaaaaaacacactcgtggcaatcagactttttaagacaatgacatactttcttcatgattgttttttcctcctttaagaggattatgctgttttagatatccattttgactttcacaactggtacttttttttaatcacccatttggcaaacagtgttgggtgactcagaattcattttgaatttgtatgatggacttttattaaaaggggtgactcagctcgacttttctgtcatgattttcttttcttgtctttctcctgtagatgcactgaatattaattagttctattgatgtgacttgtatttcttctgacctccttttccttttctcaagagccaggaggagtataaaattgggaatctggcctggtgcggtggctcacacctgtaatcccagcactttgggaggccaaggtgggcggagcacaaggtcaggagtttgagatcagcctgacccacatggtgaaaccctgtctctactacaaatacaaaaattagctgggcgtggtgtgggtgcctctaatcccagctactagggaggctgagggaagagaatcgcttgaacccaagaggcggacattgcagtgagccgagatcgcgctactgcactccagcctgggtgacagagtgaaactccgtctcaaaaaaaaaaaaaaaaaaaaaaaaattgggactctatggcaaagtgaaggaccctgtacagagaaaacatgacttaattcaggagcttaaagggtttttttgtttcttgttttttgtttttttttaggtcctcccaatttgtactggactttggaaatctgaaagaagatttattctcctgttattacctttattgtctagatcaggggttggcaagtttttctgtaaagggccagatagaaaatattttaggctttagtaaatattttacagtgcctgtcacaactactcaagtccactgttgtggtgtaaaagcagccacggacaatacgccactaataagcacggctgtgtttcctaataaaactttgtattacaaaacagagcacctggccatatttggctggcaggctgtggttggctagcccctgctctatactcatgtaatacatagatagtaaatttattctctgagcttttctttctgtctcttaccatgtgtgctcaccagccagtcacacaatatttatggaatttttctatgaaaaatatagggccttttcttgttcttgtgtagcttactgtggaaaggggctactgggtgaacagacacggtggtacagtagttgataaagggtgatagaagagttttcaggttttgagtggtcgaggatgcccggagcctggagcccagtgtacaggaggaggaacagaagaggcgtcttaacaaatggtactgtggccagatccaacacatggtttctgncctcccaatcggtgcagactacccgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngagaccagcctggccaacatagtgaaaccccatctctactaaaaatacaaaaattagcccggcatggtggcttgtgcctgtagtcccagctactcgggaggctgatgagggagaatcgcttgaacccaggaggcagaggttgcaatgagccgagaccgtgccattggactccagcctgggtgacagagggagactccgtctcaaaaaacaaacaaacaaaaaacaatatgacaaatagagaatttatatgcttctgtgaagaacagaattcctattttacagatgtctagaggtatcctaacctgccagtaatgttgctttagaatttagcagaatttgaaaaatctaaatatggtagaaaattaattatatatctcatgttaaaatttataaaataaattattacagcgttaacttaccaataagcagcttttaaattctaggctttaaaagtatcctgcttacataataaacataataaagtttcaaagatgagcaaattaacttttcagcaaaagctttacttgagtgtatcttggcttcttttttgacagttgtaaccttaatgttacagtaatgttttctttttctgggaatcagataatcttctgtttggaaaaatatttttggtgaacattttattttttaatggaaacttgtattatgtgatacgcaagtgttcaaacaatttataagagctaaccatcaccacccacatataagattatacaggaatggaggaattttggctgacttactgtatataattattgagccaacgtagatggcagactctaccacttatgatttagggttttgaaacagtattttaaagtacaaataatctcaaaattggtaatctataaagcatattaattattgtattgacgaactaaaaatagatttaaccttcctccatttataaggatgcatccattatataggttctgtacaaaattgccaagcttacttttctctttttctttttttacaacaaaatgtatttaaattttttgataggaaaagccattagctttccgtaaactgttgcacaatcactggaaatgctgagtgagagtctttctgaaaactgtagaaggctaaagataatgaaaagtgtatctcttcattgtcaagtcctttgagaatctgataggctctatctagctgcaccctgaagagacaagattaagttccaggcgctggacacagaggaacaggctcctaactggagggctttccacggacaacagagcgcctggcagtctcttcgagattaaatctaggctgtgtgggagggtgggccccccagagcttcattccactttgtgtgggtcctgagagtagggtgtttgcttaaatggcaagaaaatggccctttttcctgataattactaatatctgccatgtaaatttctttataaatccctgaaagaaaacaacctatattagaaatcagctcttacttctgacaagcaatggagctttctctagagcgctttacaggaaatatctcagctgactttctgaactggtactctctgagagccagccggcccttgactgatagccctggataaggaaggccagataagattctttgaagactctcagctaaattcagagccccaaatcctacatacatttggctactctgtagtagtggattggtggccttattttacaggctagtcagttaaggtttgagatgcgcttagttgctctgcttgtgtggaatagaaaagggggattgtaacattaatacagggccggtcgggattttagggtgcaggtaaggataggtcatagggagaatgaagattctagcaggttggttttttgttctctctacagtgggaataatgtaagaagcagatagtttgttgcccccaactctttttccatgttctcttctatcaaaatgatctgtacaaaaaagttctgtttataaaaattttagtctttcatctttgcagtctgttatcatctttatgaattttttgactcttccaccttcctggcaccacaaggctagcattgctatatttattaaagattcagatttggaattttgtcctcttatcactacttcctctgccatttgttagctctggttcttattgtatcttacatttgaaaacctttgaagcttatggggtatatcaattctttattttctcaactttttattatctaagtaccctcaattatcaagttgtttaaaaaataaataaaatacataacagaaattgttctgttacgcactaataaattgatcatttataaaagtagatccaaccccaaatccctctccttttaacacagaccatctgttattatccaccatcctttagtttggcagggtcagtttatcaatgatttttggatgttttaaaccacatgacacaaatgcagcataaagtgaacttgttaatcatgcagaagttggagggtttcttgaagtcagagaaggtgatggtggagaacttgaattgcatgaactgtaattgatttgtgagcacctcgtggagttagctaatacagtaagaatgatgatgtgacagatgtatttaaaaaatgaatttgcatattaggattgtcagaggcctttggggaaattaatgaggttctcaaatactacaaaaaaggaccatcttttcataaaaggtgatatgatgtcatatatcatggcaccatatatttttgttaaaaattagcactgaaaataattttaattttaataatttgtgcatctttccattatgataaaaattgttttaaattaaaatgacacccttacttaatttttaatttcttttttatggaaaaaaagtgaatctttttttcataatttattctgaagtattggtacctgggtatatcagtcagaccttttgcaggaactggaaactatgctaggtgcatgaaacagaagtgtttactataaaatcaggtgttttatgggaagtttggaaggtttgaagcatctgtgtctaaggtgacacttttcaaagaaatcaaatacaactggattatcaggggagttaaaacttttgaggccaccattgcaatttgtagagtggacatttgaatgttgttgtagagaagctttgttttcagaccttaactgctaacagagcagtccagacacgggaatatgatccttacctcattcgactttctaaatctttgtgtggttgcatctaattggatgaatctagctgcaagggagtctgcaaatgtagtttttctttgtatttctgtaaacgggaaggggaggatggagattgcgagagccaatccaaggtaaccaaaagaaaatttaagtggattctacttaagagatctttttctggtactgggtatcatgtgtaatgatgcagttacatttttgactggctatgtaaattagaaaggttaaaaaaaatcccaggaatcttcacagtcactagtattatgacctctgttaccactaacttgggtgccatgtggaactagctggcctgtgatgtggggcagaaatccatgtagaaagaggtgtaataattgttactatgctaggccattattaggatcattttctttttattactattgttgttttttatatttttagaggcagagtctcactctgtctcccaggctggactcctgggctcaagtgctcctccagtctcagcctctcaagtagctaggactacaggcacacaccaccacacctggctatttattttccatttttgttgagatgaagtcttgttatgttgcccatgctggtctcgaactcctggcttcaagtgatcctcccacttcagcctcggaaagtgctgggattacaggcatgaccactgtgcctcatcagaatcactttttcctctgtgtgtgtatgtgtttatataataatatcctttaattttatgctattttcattatattcaaatttatgatgaaacagagctggcataggcaataacatttacaaactgagttttggaaaactcctccagaatctacttcttacatgctatttatcaactaacctttttatataatcatagttcatgaaaaaagaaaaccctagttatgtaccagcataactcaagaaataccttttaatgattaaaggtcacctttaagagtatgctggaaaaacgaaaatgatctgtgcattttactaatccagtcagaaatactaattgtactccttattaagataaatttgggtgctggttttttggcttctggtttcttcttgtactcactatcatcatacaattggagaatggaattatgtatagtggccatacaggatgttaaaaatggttatgttccattgtgaaagacagtgtggcaattcctagacctaggaacagaaatagcatttgacccagcaaccccattactggatatgtacccaaaggagtatacatcattctgttacaaagatacattcacacatatgttcattgcagcactagtcataatagcaaagacgtggagtcaacccagttgcccatcaatgataagctggataaggaaaatgtggatatatacaccatggaatactatgcagacacaaaaagcaatgcgatcatgtcctttgcagcgacatggatggagctggaagccgttatcctcagcaaactaacacaggaacagaaaatcaaacaccacatgttctcacttataagtggaagctgaacagtgagaacacatagacacaggtggggaacaaggcacactggggccttttaggaggagtatggggagggagagcatcaggataagcagctaatgcatgctgggcataatacctaggtgatgggttgatgggtgcagcaaaccaccatggcccacgttgacctatggaacaaacctgcatgtcctgcacatgtatcccaggacttaaaataaaattaaataataaaaaaaggttattttttttcccactatgggctacttaaaaactacatggcaatatagtttttccccacaaaatacataacacaaaatacacaaaatacaaactgtgcattacaaatactttagtcttttgtgtagatgttggtgaaaaataatgagcttttaaggggagagtgtgtatgtgtgtgtatatatgcctgaataacatataaattctcaaaaatcttgcctatgaaagtgtgttacattcctttattagttgaaaagtcaattatttttcagtttcctttcatagtaagggctacttcttttttttttaacaatagaaaattaaggaaatcaaccctttgtaggggttgcgtttctaaatcactggttgtctccagctgtcagcaaacatcatgattgaccttcagcaagctaaatgttgtaccaaaagatatagctcccagggaagaagtcacagaggttaggattcagactactaaatttaccagggggcagtggcgtgtgcctatagtcctagctactcgggttgctgacctgggagggtggcttaagcccacccagttcaggcccagccggcgcacccccccagactctcctgcggaaaaaagaaggactttcaaaccacatctctaacctggcgcggcgctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntatgattgtttgatgcactctacaccccgtcatcaacatgaggtatttctcctaatgcgatccctcctccagcccccccaccccaggacaggccccggtgtgtgatgttctgctccatgtgtccatgtgttctcattgttcaactctcacttatgagtgagaacatgcagtatttggttttctgtttgtgtgttagtttgctgagaatgatggtttccagcttcatccatgtctctgcaaaggacacgaactcatccttttttatggctgcattgtattccatggtgtatatgtgccacattttctttatccagcctatcattgatgggcattttgggtggttccaagtctttgctattgtgaatagtgctgcaataaacatatgtgtgcatgcgtctttatagtagaatgatttataatcctttgagtatatacccagtaatgggattgctgggtcaaatggtatttctggttctagatccttgaggaatcaccacactgtcttccacaatggttgaactaatttacactcccaccaacagtgtaaaagtgttcctatttctccacatcctctccagcatctgttatttcccaactttttaatgatcgccattctgactggcatgagatggtatctcattgtgtttttgatttgcatttctctaatgaccagtgatgatgagctttttttcatatgtttattggccacataaatgtcttcttttgagaagtgtctgttcatatcctttgcccactttttgatgggattgtttgtttgttttttttcttgtaaatttgtttaagttctttgtagattctggtagattctggatattagcccttttgtcagatggatagattgcaaaaattttctcccattctgtcggttgcctgttcactctgatgatagtttgttttgctgtgcagatgctctttaattagatcccgtttgtcaattttggcttttgttgccattgcttttggtgttttaatcatgaagactttgcccatgcctatgtcctgaatggtattgcctaggttttcttctagggacttttttttttttttttttaatgaagtcttgctctgtcacccaggttggagtgcagtggcgcaatctcagctcactacaagctctgcctcctgggttcaagcaattctcctgcctcaacttcccaagtagctgggattataggtgcccaccaacacacccagctaattttttttttttttttgtatttttagtagagatggggtttcactatgatggccaggctggtctcgaactcctggcctcaagtcacccacctgcctcggcaggttttaggtcttgtgtttaagtctttaatccatcttgagttaattttcatataggatgtaaggaaaggggttcagtttcagttttctgcatatggctgccagttttcccaacaccatttattaaatagggactcctttccccattgcttgtttttgtcaggtttgtcaaaggtcagatggttgtagatgtgtaacattatttctgaggcctctgctctgttccattggtctatatatctgttttggtaccagtaccatgctgttttggttactgtagccttgtagtagagtttgaagtcaggtggtgtgatgcttccagctttgttctttttgcttaggatgacttggctatatgggctcttttatggttctatatgaaatttaaagtagttttttttttaaattctgtgaaggaagtcaatggtagcttgatgggggtagcattgaacctataaattactttgtgcactatggccattttcacaatattgattcttcctatccatgagcatggaatgtttttccatttgtttgtgacctctcttgtttccttgagcagtggtttgcagttctccttgaagaggttcttcacatcccttgtaagttttattcctaggtattttattctctttgtagcaattgtgaatgggagttcactcatgatttggctctctgtttgtctattattggtgtataggaatgtttgtgatttttgcacattgattttgtatcctgagactttgctgaagttgcttatcagcttaaggagatttggggctgagacgatgggattttctaaatatacaatcatgtcatctgcaaacggagacaatttgacttcctatttgaataccctttatttctttcttttgcctgattgccctggccagaacttttaatactgtgttgaataggagtggtgagagagggcatccttgtcttgtgccagttttcaaaaggaatgcttccatcttctgcccattcagtatgatattggctgtgggtttgtcataaatagcttttattattttgagatacattccatcaatacctagtttattgagagcttttagcataaagcggtgctgaattttattgaaggccttttctgcatctgttgagataatcatctggttttttgcactggttctgtttatgtgatgggttacgtttttgatttatgtatgttgaaccagccttgcatcccagggattaagcaaatttgatcatggtggataagctttttgatttgctgctggattcggtttgccattattttattgaggattttcgcatcgatgttcattagggatattggcctgaaatttttttgttttgttttgtctctgccaggttttggtatcaggatgatgctggcctcacaaaatgagttagggcagagtccctctttttctactgtttggaaccgtttcggaaggaatggtaccagctggtctttgtacctctggtaaaattcggctgtgaatctgtctagtcctgggctttttttggttggtaggctagcaattactgcctcgttttcagaacttgttattggtttattcagggatatgacttcttccttgtttggacttgagagggtgtatgtgtccaagaatttatccatttcttctagattttctagtttatttgggtagaggtgtttatagtattctctgatggtagtttgtatttctgtgggatcagtggtgatatcccctttatcattttttattgtgtctatttgtttcttatctcttttcttctttattagtctggctagtggtctattttgttaatcttttaaaaaacaccagctcctggattcgttgatttttttgaagggctttccatgtctctatctccttcagttctgctctgatcttagttatttcttatcttctgctagcttttgggtttgttggctcttgcttctctagttcttttaattttgatgttagagtgttgattttagatctttcctgctttctcctgtagacatttagtgctataaatttccctcttaattactgctttagctgtgtcccagagattctggtacgttgtatctttgttctcattggtttcaaagaacttatttatttctggcttgatttcgttatttacccagtagtcgttcaggagcaggttgttcagtttctatgtagttatgtggctttgactgagattcttaatcctgagttgtaatttgattgtactgtggtctgagagactgtttgttatgatttccattcttttgcatttgctgaggagtgttttacttccagttatgtgttcaattttagaataagtgcgatgtggtgctgagaagaatgtatattctgttgatttggggtggagagttctgtagatgtctattaggtccacttggtcctaataggaccaaagatttcaagttctgaatatccttgttaattttctgtctcgttgatctgtcaaatgttgacagtggggtgttaaaatctcccactattattgtgtgggagtctaagtcttggcatgagtcttaaacatactcttactgaaacaccaggggttcagtctagttcgtgctgctcgccgcacagaaagccaatcactgagatgacaagtattgccaaggaagaaggctttaatcatttgctgcagccaaggagatgggagctcagtctcaaatccatctccctggctgactaaaattagaggtctatatagtgggggagaaatgtagcaatgtgtaagaaaacaagaactagcatgcagcaaggagtcaatcatggtgaatgaggggtctggtgtggtgagctggtgagttttagttctttgatacttatttagaggcctgaaggtgctttttggaggaaggaactcagataaaacaaatataagtttcaagcattaagaccaaatgatcaatttctatgttcatcaaaaagatctgtctatgggactattgggtcagtttcaatacctctgttaaaacactgagcaaacaataagctattctgacacaggggcaattcctaagaaacgaggcttaaaaataatattgcagtcatctctggcagtctgaaagctgtgtacatacccaaggctgtaccctcacaggagcaactggagagggaaaagcccagtggctagtttccagatgaacatggtagaaaaaattggtaaattccctgaatgtgatagcagactccaaggcacccctcagccccagtggttaagggtaaaaccctaagggcccaggggccttaagaataatctttcactgataaatggctcatgcccattcaggggtgacccataggtagccaggctgaaagattaaaaacgtgaaggaaaaatgtgtgcaggaatgtcaggctgctcactgagccagaaacagatttcactgaatgagcctagtgaagtaaccaagtaaataaataggcaaaccagaaacacaagaagctctggggcagggaagggaaaaatcaatatctggaattgataaaatataatgcgtgaaatgtctagttttcaacaacaaattcaagtcatgtaaagaacaggaaagagtggcctatacacaggaaaacaaacaggcaacagaaacattattcaaaaggacccagatgttagacttaacaaagaactcaacgcacccattaaaaatatcttcaaagaactccaggcaactatttgtaagaaatcaaagaaggtatggggacactgtcaagtagggaatatcaataaatagattgacattataaaataattctagagttaaaagtacaataaccaaaatgggaaaaaattactagatgatttaacgatcgattagtggtgacagaagaaaataatcagcagacttgaacatagatcaatagagatggcacaatctgaagaacatacacagaaaagaatgaaaaaaactgacagcctcagaaaaatgggggtacaccactaagggaccagtgtatgtgaactatatccaaaggagaggagagagaaaaggggcagaaaaataattgaatgtatgtggctgaaaacttctcaactttatgaaaaacattaatacacacatccaagaagttcaatgaattcctcatagataaacataaagagatccacaccccctatatcatagtcaaattgttgaaagctaacaattaaaaagtatgaatgcagtgaaagaaaaacaacacatctcatacaagggaaccccaataatactaacagcagacttatcagaattaatgaagaccagaaggcagtaggatgacattcaaagtactgaaagcgaaatgttcaaccagtcatcttacatccagtgagagtttttctaaaatggagacaaaagacagacatttccagataaacaaaaactgagagaatttgttgctaaaagacctggctttaaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntcaaaaaaaaaaaaaaaaaaaaaaaaaaaaatagaaaaagaaaaagaaaaattgcctcattgtagtaaaaaaattaagtgcatgtcaaatacaatactaaagaaaaagtttttcccctataatatatacatatatattttactttgtatccatgaaaattaaaaaataaagggcagtggcagcggagatggaggagggaggatgggacaagacgctggtgcagtcccagcagcccccagcggcggccctcagcggcatggatgagaagccaagcgcaaggagcggcgggatgcgcgggacaaggacaaagaacaggagctgtctgaggaagataaacagcttcaagatgaactggagatgcttgtggaatgactcggggagaaggacacatccctgtatcgaccagcgctggaggaaatgcgaaggcagattcgttcttctacaacttccatgactttggtgaccaagtctctcaaatgtctgcgtccacactatggcaaactgaaggaaatgtatgagaacatggcccctggggagaatgagcattttgctgctgacatcatctctgttttggccatgaccatgagtggggagtgcgagtgcctcaagtatcggctagtaggctcccagaaggaattggcatcatggagtcatgagtatatcaggcacctggcaggagaagtggctaaggagtggcaggagctggatgatgcaaagaaggtccagcaggagccactgctcactctggtgaaggaaatcgtcccctacaacatggtccacgacgcagagcatgaggcctgtgaccttatggaaattgagcaggtggacatgctggagaaggacattgatgagaatgcatacacaaaggtctgcctttatctcaccagttgtgtgaattatgtgtctgagcctgagaactcagtcctactgcgttgtgccctgggtgtgttctgaaagtttagccgctttcctaaagctctgagattggcactgatgcgcaatgacatggagctggtagaagacatcttcacatcctgcgaggatgtggtagtacagaaacagatggcattcatgctaggctggcatggggtgttcctggagctgagtgaagatgtcaaggagtatgagcacctgacagagatcatgtccaatgtacagctcaacagcaacttcttggccttagctcgggagctggacatcatggagcccaaggtgcctgatgacatctacaaaacccacctagagaacaacaggtttgggggcagtggctctcaggtggactctgcccgcatgaacctggcctcctcttttgtgagtggctttgtgaatgcagcttttgtttaagacaagctgctaacgatgatggcaacaaatggctttacaagaacaaggaccacggaatgttgagtgcagctgcatctgttgggatgctgctgctgtgggatgtggatggtggcctcattcagattgagaagtaacctgtactcctttgaggactacattaagtcaggaattcttcttgcctgtggcatagtgaactctggggtctggaatgagtgtgtccctgctctgggactgctttcagactatgttctccacaacagcaacaccatgagacttggttccatctttgggctaggcttggcctacgctggctcaaatcatgaagatgtcctaacactgctgctgcctgtgatgggagattcaaagtccagcatggaggtggcaggtgtgacagctctagcctgtggaatgatagcagtggggtcctgcaatggagatgtaacttccactatccttcagaccatcagggagaagtactgagctcaaggactcttatgctcgttggcttcctcttggactgggtctcaatcacctggggaagggtgaggccatcgaggcaatcctggctgcactggaggttgcatcagagccattccgcagttttgccaacacactggtagatgtgtgtgcataggcaggctctgggaatgtgctgaaggtgcagcagctgctccacgtttgcagcgaacactttgactccaaagagaaggaggaagacaaaggcaagaaggaaaagaaggacaaggacaaaaaggaaacccctgctgacatgggagcacatcagggagtggctgttctggggattgcccttattgctgtgggggagattggtgcagagacggcactatgaacctttggccacttgctgagatatggggagcctgcactccggagggctgtacctttagcactggccctcatctctgtttcaaatcaacatcctggataccctaagcaaattatctcatgatgctgatccagaagtttcctataactccatttttgccatgggcatggtgggcagtggtaccaataatgcccgtctggctgcaatgttgcgccagttagctcaatatcatgccaaggaccccaacgacctcttcatggtgcacttggcacagggcctgacacatttagggaagggtacacttaccctctgcccctaccacagtgactggcagcttatgagtccagtggccatggctgggctgctcaccgtgcctctctctttcctggatgttcgaaacattattctaggcaaatcacactatgtattgtatgggctggtggctgccatgcggccccgaatgctggttacgtttgatgaggagctgaggccattgccagtgtctgtccatgtaggccaggcagtggatgtggtgggccaggctggcaagcctaagaccatcacagggttccagacacatacaaccccagtgtcgttggcccacggggaacgggcagaattggccactgaggagtttcttcctgttacccccattctggaaagttttgttatccttcggaagaaccccaattatgatctctaagtgaccaccaggggctctgaactgcagctgatgtatcagtgggccatgcatcctgctgccaagggtggacacagctgcagacttccgggggaattgttgcctcctgctcttttgttactgagtgagataaggttgttcaataaagacctttatccccccccaaaaaatataaataaataaataaataaatatgaaaaggttttctccacttgtcctatgaccctgggaccatggctcagaagtcttcttgtccatccatcttcagtaaatataatcaaaatacaattaatatttaccaaaacctgaggtaactttcagattcaaaatagctcggttcattacagtctttaatctcagttatattttggctttgcactagcagctttatttttttcaaagacaccctcttttcatgctcctgactgggtcagcaggagcctcaatatagcctaactgggaccatgagaagctcaatatcagaaaacactgatgacaatgaagccgccgtactcctacctacaggcttaaatgcagcacccttgtaacttcaaagctacagggagactctgatatatttctgatctgcatccaaagtgcattccatcagtctgtgatcaaaactgtcctcctctcatgtagcgacttcagaatgaatcagtgttgccaagtttttcacacatagcatcatgggaactaagtctaaattaccttagaaattgtggttccaattcagatttggctttgagcatctggttcccccaggccgaatgtcccaactcaaccacccaacttctgaggacttccctcagaagcaccatgtgagccctgtccaacgtgccacatagacacattctctcctgtcctaagagtaaatagtgaaatccagtctcagcaactatcctgtccccttcatttcattgcacaaaatgcaaacataggagacacttaaaattcttgttttcaaagttctcatttcaccatgatgtttaaagtatatggctgggcgtggtggctcacgcctgtaatcccagcacttcgggaggtcaaggtgggcagattacctgaggtcaggagttcaagaccatcctggccaacatggtgaaaccccatctctactaaaaatataaaaaaagtagctgtgtgtggtggcgggcacctgtaatcccagctactcgggaggctgaggtaggagaatcactagaaccgtgggggcagagtttgcagggggaggagtttgcagtgagccgagatcgtgccattgcactccaaactggatgacagagtgagactccatcccaaaaataaataaataaataaatanaataccgttgcctgttatttttcatgcagatttttcatgcagattactcatgctaagcttcactcttgtcttaggctgtcttccatctttgaatccctagtattttcttaaaagtatccctgacatctttttcaaagatctctaaaaatgtccaggattcttcataacacattcacctgacccagaagtcaggtaattagtttgttaaagaaatacttgttacccaactacatttaacaaatgtacagggccattaactaccaatagtatgaggaaagttagactctgttgtaaatgaaagaaattttccttttctaatagaagtcagtgcagcatggggaaaaagcaaactgccctgaacagcaggagactaagattctggctctgtcaaagctattttggacagaaaagtcacttcaccttttcagctcaaatgtcttcacttgccaatttaagagaactgggctatattaaatacttgtcttcttcagttttggtgtttttattattccctcataaaacagcaaattagtcaagtatgtacactgtacagacacatggcttgaaataattaaaagaggaaaattcttcccatatcctgggataggctatgtttgataggcaaatcaggaaataccttgaaaacttctgcaaaatggaagagacttcccacccaccccccacagaatttaacctggacatgagctgttctacatataaaatcctgtaaatatcatgaagtaaaattacaaagcacagaaagagaagtcactctcaggaaaacagcaggccccagcagtcattttacagactatatatccaaaggaggaaacagttttgtgttccaacctgcgtggatctgtgttgtggcaagttagggtcagagatagccagaaatgcaaaaatgcagttgaaggttttgtgttggtaatggaacttcaaagcaaggaagaaaatgaacgtaattttcttgtgcttcagtgtattctgcaaaaggaaaataaaaactatctcaccaggcttgtaatgaagattaaaacagttaataattgtaaagtgcttaaaacagagcctggtacatagaaagcttaatgtgtttttaaatactgaaaacagtttgtttgttttttaaagcaccagtacaaaaaaaactgacaatgacctgcaaactggttacacactggaatgggaaccatgattagcaaatcattcacacccagaggaagaaagaaagtgagagtaagccaatgcaagcactcaccttcacgcaatcttagccagcagaccactcaccaggccacaccctccagcagcttaccaactgaaaccaatttatctacccaataatacctgtaggcatcctatccgaaccccctcagcctccagaccttgagaatcttggctcccctcctttctaccattcaccctctctggtcttctgactttgtagcctactctttaaaccacccctttaggctgataccttcaattcccaccctcactaaccttccgatacatctacctgagcaaaaagcccaagtgcacagagctaatcacacacgtggactgacttcacctcccatcttcccaccacaccctacacccctcgtctccaaacctcagcctgctgaaatctgggcagcgctctcagcgctccactggaattcctctctactagggtattatcagtagctttcagtctttatcttgatttcttggcaacactgaatatcacttaacactccttctttctttttaaaaaatatcttgtttttttgtctttatcattctctttgtagcttttttccccttattgggtaagtatttatctatacgtcagtctcttcttgcttcccattctactggcactttccagagcataatggcttcaagtatcatctacacactgaagagtcgtcaatctctcttctcctaggctctctctcctgacctcagatacaaaaaactaatggcctttttgtatatatttaaagaggtatcctcaaattcagtaggattttaattgaaccaatcatccccacaacctgctcctcattgtgaattttacacacagtgtgaattgcatcaccctctatctaccctagtgctcagaccagaaacctggacattctccttgtctctgcccactcccttacctttacatgagcaaccaaatccccaaggcttctcacgtctaccctctaaggcagcacttcttaaagtgtggtccccgaccggcatcattggcatcacctgggaagttgttagaaatgcaaatgctctgatctcaactcagaccagctgaatcagaaacttgggggtggggccagcaatctgtgttgtaancaagcttcgggggattctgactcttggagtttgagattagtaggagtaacaatattaatccaagtataattagaatctgccttttatttaatcccaaatttctttcccaaatataagtaagattattttattaattaactattgtggtattatgagaagaaaattaagatgtatgatttgttaaaggaaaagctccaattcttgtactagtgcaactccctggatacttggaagtcctttaaaagggggaaaaacaaattgtgtgaccacatgcaatttttttttgttttttgagacagagtctcgctctgttgcccaggctggagtacagtgacataatcttggctcactgcaaccgccagctccttggttcaagcgattctcttgcctcagcctcccaagtagctgggactacaggcatgcaccaccatgcctgactaatgtttgtattttttttttttagtagagatgaggtttcaccaatttggccaggctggtcttgaactcctgaccttaagtgatccgcccgcctcagcctcccaaagtgctgggattacagccatgagccaccgtgctcggcccacatgtaatactttttaaaaataaaaggattttctactacctaactccttcatagttgagggcactgttctctcctggattgctgcaacggtttcctcagggtcttccagcatcctaaccacaaacacagcacacccctcagtccctactcctctaacacccagaatgctctctctaaaacacgcgactgaccacgtcatactcctccacctaacaccctccaccggcttcctactgctcctgagaatagggttcacttcttaaacgtttcttccagaagccttcacaaatgcctctgggtcccaggcagcatttctcttttctttttacttttttttttttttttttttttgagatggagtcttgctctatcacccaggctggagtgcagtggtgtgatctcagctcactgcaacctctgcctcccggattcaagggattctcctgcctcagcgcacacgaatccatcacatagtttcgctcgggncagaattcggnaataccatttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncatgcggtctcggacaaagccatgtgatggatctttggtgtattttcaactctggattttttttttaattggattttttttttttttgtcaaggtcatcctaaatgtttcagctttttctaagaaaggaaaagttaatataaataagaaattgagtttctaattcttttaaacttatccctttaaagaaaaatatcaaaaagcctaagaagttcccttggttttttgttagcaaattcctgtgctgtatttagtgttgtgttatggcagaggtgtgtgtgcacatggctctcagggactgcctacagcagaggacaagtgaactttggagtcggacttagctgacaggaaatcccagcctgcctctctggctttggtctttcagccatttttcatactgaacatcattcatttctgtgactcaatacataatagttattatttttacatgattctcaattcagacagtaagtacaatgagggaaaagaataaccatatgcaaagctcatggctttggtacaaccccaagtattgcattctttcttcccatagaagtataatcaaaaagtccaactatggaacaatgtcattaaaaatataatctaagctcaacccaagttttttgctgaacttgtagtaaaagacattataattcatttgaccttgatctaataatttatgtgtgatttcaaaagacatatgaggtatttctttttataaaaagctctaggtaatgaacttatattttaaattaaaacaaattctcatattaaatttctgagagtaggtttttatgaaagcctagtatctcacatgtacttttctccactacaaggctacaattgatgaaaaggttacacaggctcactggagcctactttcttctcaaatttctaattctcaatgactttacatttcaatgaaaaccattcactaaacctcccaacagcatttagtacatttttaatgtctcaataaatataataaacaggtcttaaaattctatatcccatgtgaggaaaacactttaaaaaaaaaggtttaaaaaaatgggggcatgaagcaatttctaagcaagccttataagcttgagtttcattaaaaaaaaaaaaaaatcagacactgaaaagcctagtggggaaaaacaacattgctcacactgagcctaatttttgagactattacaaaaataaacaaatgatgatgaatgaactttcttatggtaattaatagggaagcgaaaaagcctgtgtctccaagaatgaagccagactctatgaaaaggacctggagtttgtaatgtacaacttgttccataggaaatttcataagaaaaccatgattcagtggcaaccccaacaggataaaagaagtgcaaatgtgtaggctatccaaatacatgcaaacagagtcacaaaaaaaaaaaaaaaaaaaccatcgtggaaagttcctaggggggtcaaagggttatttgcagatttttaaagaagtgttcttgtgctttaattatttatttaacatatccatcatctcaaatattttaaaccctcttgtttagctattttgaaatatacaatacattattattaaccatagtcaccatgctgtgcaatagaccagcaggacttattcctcctatctaaatgagactttgtacccactgaccaagtctcccctttccccatccatcctgtcccctcagcctctagtaaccaccactctactttctacctctattactttgacttttttagattccacatataaatgtgatcgtagagattttgtctctctgtccctggcttatttcacttaacataatgtcttctaggtttgtccatgttgttacaaatgacagaattacctagttttttaattgtatttcattgtgtatatatatattatatattttaatgcaatcatctgttgatggacacttaggttgtttccatttcatggctcttgtgaataataatacaattagctttgtgctgtggcagttatcaaagccaatgaggtgctgttgcagtactactgaagccaatgaggtttatccgaggtgtgattattggtaataatgctgcaatgaacatagaagtatagacatctcttagacatactgaagtcattccctttgggtatatacccagtagtgaaattgctggatgatgtggaaattctattgttattttttgagaaatcttcacactgttttccacaatggctatattaacttacattccaccaacagtatggaagagttctcttttccccacatccttggcaaactcgtttcatgataaaaactcccaacaaattgggtatagacagaatgtttcttaatatgataaaggccatatatgataagccaagagctaacaggatactcaatggtgaaaagttgaaagcttttcttctaagatcagaaacaagacaaggacgcctactctcaccacatctgttcaacatagtactggacatcctagccagagcaattaggcaagaaaaataaataaaaggcattcagatagaaaaggatgaaatgaaattgcctctgcttgctgatgacataatcttgtatatagaaaatcctacagactctatcaaaaaactgttagaactgataaattcaattaagttgcaggatacaaaagtcgacctataaaaatcagaagcatttctatacactaacaacagtctgaaaaagaaaaaagaaaacagttccatttataatagcatcaaaaaatacttaggagaaaatttaaccaaggaaatgaaaaatctgtacactgaaaacaataaaacattgatgaaaaaaattgaagacgacataaataaatggaaagatatgctgtgtttatggattggaagactcaatattgttaaaatggccatactacctaaagcaatctacagattcaatgtaatcccaatcaaaattccagtgtcattcttcacagaaatagaaaaaataatcctaaaatttatttgaaaccacaaaaaaaaccaaatagccaaagtaatcttgatcaaaaaggacaaaaccagagaaatcacacaaccagatttgaaaatatattacaaagctatagtaatcaaaacagtatggtggtggcataaaaatagacacatcgtccagtggaatagaatggagtgtccagaaataaacccagctaactacagtcaattgatttgcaacaaaggtgtcaagaacacagaatggggaaaggacagcctatttaataaaaggtgttattaaaactgtacatctatatatacacaaaaatgaaaatagatccttatttcatccttcataaaagaataaactcaaaaggaattaaagacttaaaactataaaactactcgaagaaaacacaggggttaggaaaggttccatgacattggtcagggcaattatttctcggatatgacccaaaagcacaggcaatgaaagtagaaaatagacaaatgggatggcatcaaatcaaaatgcttctgcacacaaacaaaacaatagagtgcagagacaaccacaaattgggagaaaatatttgcaaatcatgcactgaataggggctaatatctaatatatttatatgtctaaatatataacaaactactcaataacagaaaggcaaataaccctattgaaaatgggcaaaggatctgaacagacatttctcagaagaagacatacaaatggccaactgataaatgaaaaaatgctctacatcactaatcagagaaatgtaaattaaaaccaaaatgagatactatctcacacctgttagaatggccatcagcaaaaaaagatgaaagtgttcttatactttatattcatagctagaaactagtttaactatcaggaaaaataaaatgtcaaagcaagaaataaaagcatctgtctatgtctctacttagattctactcatggcccacaccaatattcatttccccaacaaagctaaacttcttcccccactaccttcacatgctttacagagccaacagtaattaatatagaatatggctgactctcacagcacagagacaatgtttaagaagccacacagttaggggagattttagcctttcaagtttccaaaacaagttgtcctccttgcagataaaacagtccaaacatgtagtcatgctcaacttctaagagctgttagataattctcatacttatctgtactcaaaaatcagaaatgggtacagcccagtattaaaaagcatttcagctagtttactgaaaactgacggaaaaataaaagccaaactacaaaacaatcaaatctgcctcagacatgttccccttgaacttaactcccatttcttccctcttatctcactcccaactcaatcagaagtaacagttccttttagatatatacccagtaatgagatcactgggtctaatgagagttgtgttttacgtttttttcagaaatccccacactgttctccacagtggctgaactaatttatacccctgccaacagtatataagtgttccctcttctcacagcctcgctagtatctgttgctctgactggtgtaaaatggtatctcataatgatttggatttgcatttctctgatgattagtggtgtagaacattttttcgtatgtttgttggccacttgtatgtcttcttttgagaagtgtgtgttcacaccttttacccatctttaaaaattagtctttattaaatgtatgtatagctcacatttacttcagtgtttaatattataagtgtttggggtctttatttaaaagtgaccagaaatatgccatatgaacttaactcttgtttatatcaattagcctatagcaaaattcattttgttatatgttgttttaattaaagttgcaggtctcttttttttttttttttaagttcaggggtacaagtgcaggtttgctacataggtaaacttgtgtcatgagggtttgttgtgcagattatttcatcacccaggtattaagcctagtacccattagttatttttcccgatcctctccctcctctcaccctccactctctgaaagaccccagtgtgttttgttctcctctatgtgtccacgtgttctcgtcattaagctcccacttttaagtgagaacatgcggtatttggttttcttttcctgtgttagtttgctaaggataatggcctccagctccattcatgtccctgcaaaggacatgatctcatttttttttatggcttcatcttttgaccatttttttcatgaggctatttattttttgcttgttgaattgtttaagtttcttgtattagtaggtgctggataaaagacctttgtggaatgtatagtttgcaaatattttctcccatcctgtaggttgtccgtttactctgtggagagtttctcgtgctgtgcagaagctctttagtttaattaggttccacttgtcaatttttgttcctgctgcaattacttttgaggacttagtcataaattatttgccaaagccaatatcccgaatggtatttcctaggttttcttgtacgactcttacagtttgaggtcttacatttaactctttaatccatcttgagttaatttttatatgtgatgaaggttttttgggggtccagtttcattcttctacaaatggttggccatctagcaccatttattaaatagggagtcctttccccattgcttatttttattgattttattgaagattggatgactgtagatgtgtggctttatttttggattctctattcttctccataggtctatgtgtctttttgtaccagtaccatgctgttttggtctctgtaataataaattgttctaccaaaaagacacatgcactcatatgttcatcacagcactatacacaatagcaaagatttagaatcaacctaggtgtgcatcaatggtggcctggataaggaaaacatggtacatatataccacggaacattacacagccaaaaaacaaacaaaatcatgtcctttgcagtaacatggatgcagctggaggctatgtaagtgaattaaggcaggaacagaaaagcaaacaccacatggaaaagacctatggaaaagaatagagaatccaaaaataaaaataaagccacacatctaccgtcatccaatcttcaataaaaataagcaatggggaaaggactccctatttaataaatggtgctaggtggctacccatttgtagaagaatgaaactggacccccccaaaaaccttcaccactagctctaacttataactgggagctaaacattgggtacacatggacataaagatggcaacaatagacactggggactcctagatgggggagaaagggacaaagattgaaaaactaactattgagtactcttcttagtacctgggtgagaggatcaatcaaacccaaaaccttagcatcatgcaatatacccagataacaaacctacacatgtacccctgaatcaaaaataatagttgatttattaaaaaaaaaaaaaaggaagtaacagattctggattctacaccctccttttgtcccatggacaaagacttatcacagagtctgttcagggcaatgaaaaaaggaaaaaaaaaaaaaaacaaacccctttaagttatctctcttactggttgccctaagtttcaaacacctccaaagcctacagacataggtcatttcctaagaaacacacgaaggaaactaacattgattaagtgcttaatatgggtgcaggtgtgtgtacaatcagaactcaatagccttttgaactaatgagaaaactgaggactagagatataaagtaatttgcccaggtttattcagccagccagtggcagagctgagattcaaatctgtcttggacagcttgggctgccatagcaaaatatgacaaactggggtgcttaaacagaaatatattttctcataattctgaaagctgggaagtctaagatcaaggtgccagctgactcagtcactggtgagaactctcttacaataataagcatttgtgtattgaaacatatctaaacctagaaaaagtaatgtgatacactacaaactagcttggtgatagcaatttttcaaccccattataatgttatgggaccaccatcacatacatggtctgttgttgaccaaaatgttgttatgtggcatataagtgtatttcattctttgcttggttcaatcaagagcaaggaactctgttaataattttgttaaaagaattttatcctcatttaattcacagatgtgaaaaaatttcatagtcaatatgcaaactgcaaaatttgtattatatgacacataataaatgaagtctgataaatttattttcaactgtaaaaatgaaacaactggtagccaaaagtagtatgcctgtttgccagttttatttttgcatatgttgcgcattctgattttattaggtaacacatgcttcattcagtaacaagctannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncattgggaggcttgcctcaggcgcgagcgaagcatgccatggatgggacaccacatcttgctctgattgctcttgcttggatggctgcagcctggcaggtgtgtattcacaccctccctacttccatcgatgatggtgatccttaaagggcccttctcaaaggtctcccgtggttctccatgcttgcctgttataaagcttctctaacaggagactgaagaaattagaaatgccagcatcctgcctctcccagaccaaaatcatagccatagacatagttgtagagaaagacagagaactatggtcttcttggccacacatgtctagagtagagcagagctgggggtggctggtaacagagtgggttgtggctcaactgtcagagactcttgctgttatgagttttaggagattttcttgaatgactgtttctccgtttcctgtgtgcccttaggacaattttcagagattttacacaattttttgttataattctcaccagttaagttgttgttttactggggagaggattcaccaagttcctcactatctcgagaagactctcctccagacagcttgctaaagtaatacttcagacagtttcaggtcccaaagtaattttaaagattcataaattaacaaaaactaaaggtgaaatactaaacatcttccatttcgttgtctgggcatggtggctcacgcttctaatcccagcactttgggaagcggaggcaggaagattgcctgagcccaggagtacaagaccagcatgggcaacatggcgactccccgtccctacaaaataaaaattaaaaagttagccaggctcggtggcccacacctagagtcccagctacgtgggaggctgaggtgagaggattgctcgagccagggaagtcgaggctgcagtgagccatgatcataccacagtactccagcctgggagacagagtgagaccccgtctcaaaaaaaaataaatacaaaaaattttaaaacctccattttgcaaatggttatggctttaaaaaagaaacagtttattttccatttataaaaagttttaaattaaagaaactaaagacacaaataagaactttattattttttttctaaatcttaaacttatgccctcaaataacaggaactctttttcggtttaggtcacacgaagtttggttgtgaataatctgactagtgtcatcaccagcatcccttctggtagacaaaaggtaggacctatcataattgtgactatgtaggtcaaatgacgaataaaatcctcctttgccctgagatcatcccaaatcctgttgctgtccatggtaaagaaggatacatcctgctgtttcataccgtgacaccaaaataagcttcaggtccaattaatctaaattcagtcctcacacttatttctagtatgacaaacccgaggctgggtagaaggatgagaaggaaacacattcagctaagagaaatgaatataaatgatctacattcacattcctaaaactcccagacagaagcacgcaagccaggtctgctaaatgagctgcacggaaacctttgcttgtggtgctattctcaaaaatcatcccttaatcctctctacatatttaaaccaaataaattcttcaaggcccatttcaaatcatatctcctccacacagcctttatcccaagtccagcctatatactctgttctacttctgaccacccacagtttgcactatctttagtatatagcatggaacctgaaggataattcaagtatagtcacttgaattactcaaaactgtctctcttgggttgttccacaataaattagccctccagagaacctctgtgccctttctggtattttagctcatttcctctgctgcagtccctgctgaaatggatcaactgtgctgtcatcggatcaaaaaagggggcaacagtgaaagtgttcactgtttagtttttccagtgaaactaaagagggctctccgcaatagtggctccactctaaattggctgtgctgagaaaacactctcccatcatctgctccagatagaaataaaatttttttttttttagagatgagatggggtctcactatgttgcctaggctggagggcagtgggtatcacaggcaaaatcacagcacactacagcccacaacagcccaccttgtaggtgggtctacaggtacatgccactgcgtctggcattccaaagagcacttatactctattctataatctttcttcaattcctatgaattatacacagaacattctagccattaaaaattaatacccctttcttgtacatttagtccaacagaatcttttttttttttttttttttttttttgagacagagtctcgctctgtcgccaggctggagtgcaatggtgcaatcttgactcactgcaacatccacctcctgggttcaagcaatcctcctgcatcagcctcctgagtatctgggactacaggcgcatgccatcacgcccagctaatttttgtatttttagtagagatggggtttcaccatgttggccaggatggtctcgatctcttgaccttgtgatccgcccacctaggcctcccaaagtgctgggattacaggcgtgagccactgcgcccagcccagaatcttaataccgtggtatataaatataaaatccaaaagaattaactatctacactatgattaaattgtatgcaattccaaagcacgattacctaaatataatttccctaatatataatctagcaaggtgaaggataattagcttcattacatttcctttatattatgctggtgtattcgtgtttttttttttatttttagccatttacttagaagtataaaaacaatcttactggctaaaagaaaatttaaaagcagaggttaatattattgcattccttgaattgattgggtatgctctacaaatagttataggaatagcatcttcaaaaaaaaaataaatctttcaacatcttaaacaaatccagattccacaaaacctatgcttgtctaattatagttttgaattttgatgtggttttctatgtaaatacatttttaaggcttgcaataggaatgtattctattaaaagcagatgttaaaagttacaaagtaccaaatgtttattatgtatccccaaaacaaatttaaattgttttattataagatatttagtgtttcagacattattttatgtctttggtcaaaagacaaatataactgtccaaatcaaccaaaacacacccttgccaactacaactggaacaattaacaccaatacaaaacatgtcttctataacacaaattatcattcagtctgtcactctgcaagcactgaaatatctactaattacaataaaacatagttttcaatctaaaaatgaatatctgcaataaataaaactgcagcaaagatcaaagataaaatatgtaatctctttttccttttcatagcagccaataatacagtggcaggcaatacctgaagtgtgccactcttgttctcccagattcctcaacatagtaagaagcagccttttattggcaattactgcttcccgtaagaacacttacaggtattacgttatgtactcccatataagagcagctgaatgatgaatacttcttttcctgtcgttattacacatttttataactttttttttttttgagacggagtctcgctctgttgcccaggctggagtgtagtggcgcgatctcggctcactgcaacctccgcctcccgggttcaagcaattctcctgcctcagcctcctgagtagctgggattacaggcatccaccactactcccggctaatttttttgtatttttagtagagacagggtttcaccatgttgtcaggctggtctcaaactcctgacctcgaacatttttaaaaagtcttttttagggattaggtcatcaagcacagcggaaagactaaacatgataccaaatatctaatccttaaagttagtatctgtaataaatgtgaattctaacaaatccttttattacctgcccttataataagtagcacatttaactatgtattttttttctgatcagatttccagaggctaaagtccaacaaatatttaactgtcatactttttctcccattctctgggcaaagaattttaatctgatagattacatagaagaaacccacaaagagattcattgtgttgcattaaatggttagaccaatgtcaagttatgtactttttaaaaaatatcagattcttcttttacactcttgctgacttctatgtgcaaatttacattctgggtatcatgttcctttacagatcctttaaacatgattagacattatttacctatggcttattttacaaagatgattcttttttttaccatgggataaatgaaatgtagcatcatttcacatttggcaaggtttaaccacaatggctgtctgcctctaaccactatttatatgccacacaagcaagtggctcttacttttactgcttacggaagcttatgtatttaaatctcataccactcaaacttttatttaactgtgcaaatgtcatgttggtgaaaccacagtttatatacttgtcaaaatctgctaaaatattggccaggcgcagtggttcatgcctgtaatcccagcactttgggaggccaaggtagacggatcacctgaggtcaggagtttgagaccagcctgatcaacatggcgaaaccacgtctctactaaaaatacaaaaattagctggtcacggtggcccgcacctgtaatcccagctacttgggaggctgaggcaggagaatcgcttgaacccgggagggggaggttgcagtgagccaagatcatgccactgcacaccaacttgggcaacagagtgagactccatttaaaaaaaagaaagaaagaaatctgctaaaacatggtaaaggttttaaaagtgtctcacatacaattttatttataaccacagtcatttaaataccaaacatatatgaaaaaccaatacacattcaaattctttttttaaagtatttaattcatttttaaaagtatatattctggtctccatataaaatactgtacttacactcatataaaatattccacatccataattatcacttaattaggaagagcagaggctttgaagccagaaggctggagtctgaacccgagctcagccccttaccagcttggaaacattgaattccgctatatctctgtacctgaggtttctgtactatgaactgaggctaacggtgcctgtctcacaggggtaggagcattatatgagctgatataagtgctaagaacagtaactggtacatggtaaataatggacacaatatgacttgtggtttccatggttattattgctgtaataattactactattactactactactgcacttaccaaaatatatttccttatgacattatttgatagaagattaaccaaaaactatgcaacccttattatcaaaatcactcccaagtaaattctctcactttcctgctaatgagtgcggatgaatttttttttccctgagagagggtcttaatctgtcaccctggctggagtgcagtggtactctcacaactcactgcagcctcaacctccctgggctcagatgatcttcccatctcagcttcccaagtagctgggatgacagacatgcaccactgcacccagctaatttttatattttttatagattcagaatttccccatgttccccaggctggtctcgaaatcctgggctcaagccatccacctgccttggcctcccaaagtgctaggattacaggtgtgagccaccatacccagtgggttaaactttttaatccttatttgatcaccaaagatcctgaggtgacttagggtcattacaaacattaaaaatcactgaactaccctggaattagggaggagattggactaaacacttactggctctaaagccttccttaaaattgggcaagtgtgaatgtgtgtgaatgctaaagcatgaacatcataagagatcactttttctttttctttttcttttttttttttttttgagatggagtctcactctattgcccaggctggagtgcagtggtgcgatctcggctcactgcaacctctgcttcccgggttcacgccattctcctgcctcagcctcccgagtagctgagactacgggcacctgctgccacgcccggctaattttttgtatttttagtagagacggggtttctacatgttagccaggatggtctccatctcctgacctcgtgatccgcccgcctcggcctctgaaagtgctgggattacaggcgtgagccaccacacccggccaagatcactttttctattttcctctttattccttaagagacggggttctacatcagacaattctaatgtcaaagtgtcacactcatttggttatgaaaaactatgtcaaactatattctgataaacttctatgtaccttctcagctaatcttttgtgtaatgcttcaaagtaaagaaggagaaatgaagaaatcatcatagaacaaacttaaatccatgctccatgagaaaagtataaaatgctttgtcttaagctctttggagaaaaaaggatttggaaatacaagatattcttcatttctatcagtctgagcatccagagaaaataaccctctttctccttatgccaaacggcaatgtgcccagctgaggaatcttctattatcacactgaaaacgtctctgccatgacggtaaaaacagcacaggcacctcgttaaacaccagtgcattcaaccacagtcttactttcaaggagaacagagatcacagaaaaattatttggctatttttaaatatttaaacaaaaacagaacttccagcttccattgtatataagaagaattgcttgaaatacctccataaacatatgccacggcattaaaggctattaattttactggaaatgaatcataaggtaccaaaatgtctgcacatacaatcagtgcacatttcccataaaaaggggaacaatttgcatgcatcctgaaaatacactttccttaaacaaaattttaaatgtctcaagtaaatggaatggacagatttttcttccaaatacaatgaagttaaataaaactatgtttcttcagcaatccatcactttgtagtgatttttctctaacatacatttaaaaaaatcttttgtccctccaaaaacagatctgaagtgcagagctcagcaccaagacccaggccaaggagttgctgaataggtttatctggagtcatgatggagactgtcccataaaccctgtttgaagctgaaggtcaacagtagcgtcactatcaataaacacaaatgaggatattggcaaagtctcttaccgacaatttatgaaatgccttaatgacagaatgaggagattatcttaagatgatgatgctcaacacagaggcactgtttatatgctgagaccccttcatgccaactagtttaatgtgaagtttgatgatttacatgtaaatcatatttcagtaagctaaaacttaatactatgtcaaacagagaatgaaatcctacagtcttgggcaacctgggagacttcttccttatctgtattcccttagccaaaaaaacaaacaaacaaaaacaaacaaacaaaaaaacacaaaaaaacaaaaggggtgctggaagtaaggagagcatggctcaattttctgagtatttggtccctggacgagagaggctccatgccacatctacacgtttgtcatgtgacagttccggctgcctgactcttctcaatgtaagccccacccatattcctcgcccagacagattttacatctctgacacaacccaaaagaggagatacgatgattcagcctcagtgtactatatgcaaatataccaaggattttttaaatatttatctctgccagagaaaatatgattttccattactccttttaacattgtcaccctggacttactgattattatgaaatgagtaaccttctggtttttaatcactactggacatagccactgatatttataaagtcttcatcccttagtcagctgattaatttaataaatatttattgagtgcctacaatgtgccaggtgccatctcatttactatgttactgacctgaggtcaccagcaaccagtgataatgcaacaagcatttctaaaagaattcaactgcataatgtttgggtttacattattctcagattaataaaatgcctggggtgagcctgctcacccagccaactacgggtcacaggggtctagctgtcccattgaaatccatcttctcctggctggatttggcaatgtccatctaatgaaaattgtctgctctgaaataaatatcaatggaaacaactccaggtcatgtgcctagctattacttccaggtcagaggcacaaattcgaggaatcaccccattagctggaactcctatggggctactgggcaaggagaacactggggatatcagcctcatttttccctctttgcttcccaagactgagctgcttgccattcctttgcttccaagacctctttcctgccaccctgcaaaagttaagaaatgtagccactctccatttcagggtaaagacgggcaccattagcaattctttctatctggagttcattatcatgaggttttactctactttccaaatgacacccctgagaaatttataacaatatccctttatgacaagtccaatggatatatcagtagaaaaaaattgcttggaaatgtttttcttgaaatgcaccaatctttctttttatgactcaaaaatgtcacaggttagatcaaaggatctaacataaaatagagaggttagaaatggatacaagatttctcttccacatttcaagtacagtgtgactgtttaactggattctaatggcatccacttcagatataatgcaacaaaataccaaacacattatcagttactaaggattggatttcttgattgttttcctttgcccactctttcctttctttccatccataggagcactcagatggacataaatcaaatgaaaatttatttctaggcaactaattctggaaagaaacaatggcggatattttggaaatatgtattatgatgttacaaataaaagttgggacattatcactggatggagataattatgtgttgttctatttgggctagtcttacttaaccacatgttctaaagttctaacccttgctgtctgcctaagatccatggaggtttacacagcccaaaaactatttcaaagttttctctaatgacagttcacacttatgccagaaacactgtccaatttcataaagcacaaaaatactcacttgtttagccttcgtctttgagatagtatcagagataggtttctttctctcctttacagcagttttagaaaataattcctcaaattcatgacaatctatggatggctcttcaattttttcccaaataagtgaagtactggagtctctaaaattaagcaaaagaacaataaaacagccatcaaactgaacttacatcacagcaagcataaatagaaaggaagaaggggagggaggaaggccagcaggagggaaggagggaaaaagaagagagataagaaggaaaaaatattctttttctttaagaccaccatttgtttgatcaacttaatgggaaagttgaaataccatccatgaatgaaatttttccccagaaataatttccaggaagcctcagtcatcaggccactgtataaaaatgtaaaaagaaaacttatctcagtgtgggctagggaggcaagaaatgtctgatctctcaaaatcccaacttgacttaacaactttgcagattattcatgggttaaaaaatacatatttacaggatggcatcctctccctcctactgcctcccatcaccccattgttatttgtcttctcagtgactgcagattcgtgtagtaccacctaaaaagggtaaacacatctttttaaaaagcaaaccaacaacaaaacacaagaacttcccctgtcatatctagagaaaatgactttggggcacagtttaaaatgaatcacccacatacataagtggcgtggtaacaacacagaagatgaactgaaaaattaaaagtcatcagaatatacagattcttgcccccgatatggtgtagtgagtagaaaaatccatataagaaatgaaaacgaacctggcaaataccctgtttctaaatgcatgccacatcacacaaaggaaattcacacaaaggaaattcaaggaatcatgaaacattagaacaaggaagccccagtgaggtttacagtgtgcgtttttccctgccacctacacattccacagccaagtatcacagcattgtctcaatgactaggcaacacgggctatagatgacacataaatgtataaagtaccaccagaatacctggtggaaagtattcttttatgatgatcagtcccagctatgattgtatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntggaaaaccaaacattgtatgttctcacattcataagtgagagctaagctatgaggatgcaaaggcataaaaagtgatacaatggactttggggacttggtggaagggtgggagggggtgaggaataaaagactacaaattgggttcagtgtatactgcttgggtgatgtgtgcaccaaaatctcagatcaccactaaagaacttactcatgtaaccaaataccacctgttccccaaaaacctatggaaataaaaagtattttttaaaaatgacgctggaggtctagataaattctgtggccacttctatatccaaaatcaatgatgtttatgcttctgtggacttctcagtggaggcatatcctgagagaagtcaagtttgctctagaggacacaagcgattcacacatgtaaatcctgtttgcagacaatacaccaaggcagaataggactttacctgcaggagggcacaacctcttgctgctttagacttggaaggccaccatattaaaaaaaaaaaaaaaaaagtgtacagggtttcaaagggactaaagcaatcacttcacatcctaatgggataaagtttctgcatgattctgtatgacccttcaactactgcctttcatgccatgttattgctagcgttgcatgttaaagcaagcaaattttcaatgaactctcactggctttaccacagacacagatgtcaggcggactcttgttcataaggttctcattatcaactcaacccgagttgaatatttgactgaaggaaagaaccatatcattattttcagatttcatcctatgacttaactgggctacatctgccagctttagaaacaccaagtactgcctggcgccagccttggctttctcttctatgactattaccccaaatgtttcagcttgaggaagtctcctccccatcaaaacaatttaataaaaacttaaattgtgattaagagttactcatcaattgacagggcataccaagcttgtgctatttatgaattatcccacacaagattttaagcccaacaaatagaatccacattcataaaggggtaagctagaaagccagttaaatgcttttgtgattataaggtctgtgttattcttgaaaccatatttctttaataggtaaactggcattccattggataattcaatctgctttaaatctattcttaggttctagtcagcttaagggacactctgaatttacacttcattactctattcacctttgggcagtatctctcagcataaatatcataggaggatatggcataagaatggccgtcaattgtacagtagaatccaagtattaaaatattgacactaaagccatttataaaatgttaagaaccactctaattccaaacagaaaatatttctgttcatatgttgcaaacctcactcccatgtgaaggtacaaggagacaggatcctaggtggtggtcactaggtcgcgtggtcattaggtcacaagggtagaattctcgtgcgtgggatcagtccccttataaaacagacacaagagagctctctttccttctttctgccaattgaggatacatggagaagatggcagtctgcaacctgaaagacggccctcaccagaaccgaatatgctggcaccctgatctcgaactttcagcctcctagaactgtgagaaataaatttctcttatttatcacccaccagtctggggtacttggttatagtagcctgaactaagataataatgtcacatgataattagactgttagtaaggtatataaataacaccacaggcaaattaatagtaatccttccagaaacaatcatgggagatagcttgatcacacttacgggatatatactaacctcggtttacacagaaactctaagtttgaaataacattaccaacaaacatcatatccctcagcccaaattttaatattcagaagctaaattttcattttgacacttaccatgttgtatgtctttcatatctaaatgaaggctagacattagtattccaactgcttgtgatcttttgttgcttaataacttgacaacctgctcgagaaagaaagtaaaaggggaaaatgttaatttgaactccaatatgcttactagcattttaaaaaatcctaaatgaagaacaaattcagagaaaagatctcacgtcattacctaaagtattcaaaacagtgttttttttaatgtttgaagagagcttttgggttttatatcataaatcttcaaaatccaccattttcccccattattcaccatcagcctcaaagacatgttttttaaccagtcttttgagagcctgatctgtcaacaagtaaaatttctaatggtctaggaaattagtctggtctctgtaaaatcacaattacacatatgatcatttgcagcctgagatttgaagtgttcttttttctttcaatgtggagccttgacacactgaaacttcaaacacgtcagcaagtgaagcgcaaaataaaagtacacacatagagaatctgctagattagtttggttgtgtcatgaaaaatttttttaaaaaagtgctttcacttgggtcacatagaaagctcactattttactggaagttcctctcattctgcatttgacccacattttggggagaactttcctactaattaacagagggatacaagcatcaaattaaaagggctgcatcagcacaatctcaagtctggatttccagcaagtcttaaagaatcagggcacacgatcccatgcgaagggttcctaaaacagaggtgggaaggctgttacagaacagatagatattttttgaccatgttttccagacaacagcatcaccaggaaattctgcttatgaaacttatcccaggcctgatcgtgtgaggggcagcattttctatttactcccagtgttttgttttaaatctgcttagaaaaacactggaggaaaaggaaagaaagtggttatagtcaaattaccagaagtgacagctgctctaacagctattgcatctttgtaccctgtcttcccttatggtttaagttgggtcaatcaggatagtaataaaataatcatacccaactgttacacaaagtcacagagtaaaacaatattcttaatagcaataatagcactctgggtgccccagcacggatgggggtgaggaataaaacataacactttcagaaggaccaaccacgaaaaccactctgatcttttttgttgtcgttgtttgtttgtttgttttgagatggagtctcactctgtcacccaggaaggagtgcagtggcacgatctcggctcactgcagcctccgcctcctgagttcaagtgagtctcgtgactcagcctcctgagtagctgggattacaggtgcatgccaccatgcccagctattttttttttttttttttgtatttttagtggagaccgagtttcaccatgttggccaggaaggtctcgaactcctgacctcaggtgatccacccacctcggcctcccaaagtgttgaaataacaggcataaaccactgctgcctggccctgccctgatcatttctgaaagcctcttggtaaggcacgatatctcccaaaaggcgctgcttttgcaaaagaaaaagcaacatatacattttgattctaaatgttaactaaacatttttctaaattccacctagaaagatgaggttaaaactagaatatgggcctattagtacagcaaagtatttctatccatagcattgtcctcttaagagcttaaaaagtaggaatttaatatcagagaagcagaaggtgtatactctgtcctgaaaaccagtttgtaaccacaagtcagtgcaatccgggctgaatcatgctgcttgatgaaaatgcttttatcaaggttttctatcatcttctggtttctcaatgcaataggcatgtttcaatccatctatcattcgacctccctatagtattgacatgttgatcacacccttgtagtaacttctcttccagggctcctaacatttcttctcagttcttttgaagatgaattattctgatttcaccttaaatatttgcgtatcctagagttctgttctctatatcttattttcgtcgcattataacctattctcatcacattataacctataatttctcctgtagagacatacatgtctgcatcttcaacttggcagtctcctacttcgtgaaaagacatctctatttaaattgtccccataatctccatttgacatgtctaaaatgacacacatatcagttccaccccaacaccaccagccagcttcttctcctttatgctatatattgagataaaggtaccactattcttcatccacctaccaatgttaggaatctagaaaccagtcaagactcctacctccccttcacctgcatactctggtctggtatcaagtcctgccaattacagctgctctgtatttgcggaaaccacccctcctccttaactctattttcagcatttagttctatactttatcttacatagataaatgcaaaagatttctgattgatctcccaaactctagtccagtggttctcaaagtgtgttccttggacaaatgggcaccatcatcacctgggaagttatcaaaaatccaaggaaaagttagaggggagtttcaagatggctgactagaggcacctggtactcgcctcctccacaaacaagaactgaaatagtgagtagatatcacaggtcaaacagatcacataagagagaacattggaattcaagaaataagtgacaggtaacatgggaggcaaagaagaagagggaagtggggcagtctgctcagccaggatcggctgggagctagagaggttctctaatgtgattaaggttctccaggctccctcatgtggtaaagttaagtgagcgaccccagcagcccacatttccaccacagactcctgcaatcgtagccatgggagaaccccgcggcctgcacaggccctgaggctaacattgggagccacccggagatcaggcagcggcacccctccagagggagcgcgcactgagttccactcacctgctgtgtcacaagcagttacaggagggcactgttttgaaatgtcagcccccaccagactgcatcctggcctgggcccaatagcccctgcatctccacatccccggggctcccctgatatcctccaaccgcagccagggccaaagcacaagcctttgccaacgaccctgctgcccctagcaacaaggcctctgtgcatttaagagcaccctgaggatggggcacccctcttgcagccgccatatggagccacagcacaagctcccccagtcacctgtttacaactgctgccatggtaactccaccctccccagcagcagggcctcagggccaccacacagtcgctgccacccccatccagctattctgccaggggtaggggggacctggggtccacccctatcctacttaccacagccagcacccacacgtaccacctaggggccagagaacaggaccatttaaccatgttaaaacccatcccactcccccagagcccaagaccaccacccagaggcttgggaatggccctgccccattcaccaccattgacacctgagcactccttctgagggcctgaggacaaacccacccaacctactgctaacaccacagctagcacccacccacccacaccatccactcacccatgccacctgcaagccttgagactgccccgcgtggcctgtcatagccactgcaacaccagcgcaaaccacttgggagcctaaggcttgtctcaccactactactgcgtccctcagcacgatgcccactgcctaggagcctaaggaccctcccagccacccagcccacttcttccactgctggcacccaagcaagccacctggaggtccaactggcctgactgaacctgctaatatcagggccagcatacaccaccctaagaccaaaggacaggcatgctcagcctgccactgccacctcaagcacctgccagtgattgaggactggcccacctagcatcctcatcaccagcaaaactttaccatgacctccactaacaaccacaccctaagccactgaggaaattacaagcactactgacacttacagatgaaaaaaatcatacagagactacactgctgtattcatccagaatcaaaggcaaaatgctacatcaaaccaacaccacagatacatcttcaggaaaaagtcctcccctataaaagcaaattcaaaaaagttggaataagtgactgttataccagatgcacaaatatcaatgtaaataaacaataaacataaaacaaagagatattacacctccaaatgaacataataattctccagcaacaaattccaatgaaaaagacattcatgaagttccagaaaaagaattcaagataatgatattaaagaatctcagtgatatacaagaaaattcatataaacaatacaaagagattagaaaaacaatttaggatatgaatgagaaatttatcaaaaagatagatatcgtaaaaaagaaccaaacagaaattctgggatggaagacttcattgaataaaatacaaaatacatttgaaagcttcagcaataaactagattaagcagaagaaagaattttagaacttgaagacaggtcttttgaaataacctagtcagataaaaataaagaataaagaataaaaatgaatgaacaggccaggcgtagtgtctcacgtctgtaatcctagcactttgggaggctgaggcaggcagataatgaggtcaggagattgagatcatcctggccaacatggtaaaaccccatctctacttaaaaaagtaaaataaaataaaatacaaaaattagctgggtgtggtggtgcatgcctgtaatcccagctactcaggaggctgacgcaggagaatcgcttgaaccagggagtcagaggttgcaatgagccaagattgcaccactgcactccagcctggtgacagagtgagactctgtctccaaaaaaaaaaaaaaaaagtgaacaagactacatcacacatgggacaccataaagcaaacaaatatttgaattttcagtgtcccagaagatgaagaataaatgaaagggatcaaaaacctacttagtggaacaatagctaaaaaaaattcccaagtctagtaagagatttagacatacagatacaggaaggtcagaaatccccaaatagatacaattcaaaaaggttttctctgcggcacattagagtcaaactgtcaaaagtgagaggcaaagaacaaattctaaaaacagcaagagataagcatctagtcacttataagagaaccaccccccccgtcagaataacagctgatttctcagcaaaaaccttactggccaggagagaactgaatgatatattcaaagagctgaaaaaaaaaaaactgccagcccaagatactcttcccagcaaagttaccctccataaatgatggagaaatacagtctttcctagacaagcaaaagttgaaggtactaatcaccaaaagactgggcctacaataaaagcttatggtagtcctacacctgcaagcaaaagaatgatatccaccatcatgaaagcatgcaaaagtataaaacctactggtagagcaaatgcacaaataaggaagaggaaggatccaaatgttgccactatagaaaactacaaaaccacaatgatgaacaataagagaaaaagaaaggaagaaaggatatacaaagcaatcagaaatcagttaaggaggccggaaatagtggctcacacctgtaatcctagcactttgggagggtgaggcaggtggatcacctgaggtcaggagtttgagaccaacctggccgaaatggtgaaatctcgtctctactaaaaatacaaaaatcagccaggtgagatggcgggcgtctgtaatcccagctacttggaaggctgaggcaggagaactgctttaacccgggaggcggaggttgcactgagccgagatagcgtcactgccctccagcctaggcaacagagtgagacttcgtctcaaaaataaataaataaaacaaaataaaatacagaaatcaattaaggaaatgacttgaatttgcccttacatatcaataataaccttgaatgtaaacagattaaactttccgcttataaagaagatgataacatgataaaagtttcaattcagcaagaggatgattgtacccaacaacggagcacacagatatataaaacatattattagatctaaagggagagacagactcaaatacaataatagtaggagacatcaacatcacactctcagcaacagtaggatcatctagacagaaaattaacaaagaaatctaggatttaaactgaactttagaccaaatggacctaacagacatttacagaacattgcattcaacagctagagaacacacattcttctcatcatcacatggaacattcttcagagtagataatgtgttaggacacaaaaatatcaacacattttaaatatcgaaatcatatcaagtatcttctcagatctcaatgaaataaaagtagaaatcagcaacaagaggaactttagaaactgcacaaatacatggaaattaaacaacctgctcctcaatgactactgagaccaggaagaaattaaggaagaaataaaaaaaatcctagaagcaaatgaaattggaaacacaaaacaccaaaacctatgagatacaacaaaactagtactaaaaggaaagcttaaaataataaacgcctacatcaaaaaagtagaaagatctcaaataaacaatctaacagtgtatatcaaggaaccagaaaagcaagaacaaaatcagtaggaagaaataagtaatgaagattagaacagaactaaacaaaataaagactaaaaaaacccacaaagaatcaacaaaataaagttagttttatgaaaaaataaacaaaattgataaactgctagctagactaaccaagaaaatgagagaagacccaaatgaacaaaatcagaaattaaaaatgagacattacaactgataccacagaaatataaaagatcatcagagactattatgaacaactatacaataatatactggaaaatctagaggaaatgaataaattcctggacacatacaatctaccaagattgaatcaggaagaaatagcaaacctgaacagactaacaatgagttgtgagattgaatcagtaataaaaagtctcccaacaaagaaaagtccaggaccagatggtgtcactgccaaattctacaaaacgtacaaagaacactaattttcctcaaactcttcccaccccccaaaaaaaatagaagagaagggaatgctccctaacttattccaccaggtcagcattaccctgataccaaaaccagacaagaatacaacaaaaaagaaaactacaggccaatatcctgatgaacagagatgcaaaaatccttaacaaaacactaacaagttaagttcaacagcacatctaaagaaaataccataatcaagtgggatttatcccagggatgcaaaaatggttgtacatatgcaaatcaataaatgtgatatatcaaatcaagagtgaaaaacaaaagccatatgatcatctcaatggacacagaaaaagcatttgataaaattcaacatcccttcacgataaaaattctcaataatctaggtatagtaggaacatacctcaacttaataaaggcatatatggcaaacccacagctaacatcatactgaatggggaaaagatgaaagcctttcctctaataattggaacaagacaaggatgctcactttcaccactcctacttaacataacatagtgctagaattcctagccagagaaatcaggcaagagaaagaaataaaaggattccaaactgaaagacaggaagtcaaactgtttctctttacaaatgacataagcttttatctagaaaatataagatgccaccaaaaaaccattagttctgatgaactcagtaaagttgcagtatacaaaatcaacatacaaaaatcactagttcctacagaacaataatgaactagctgagaaagaaatgaaggcaatccaatctcaaatagctacaaaaaataaataacttaggaataaatttaaccaagaagacagaagacctctgtaagaaaaactacaacacattgatgaaagaaattgaagaggacagaaacgaatagacattccatgctcatggacaggaagaataaatattgttgaagtgaccctactccccaaaacaatctccagatccagtgcaatccttatcaaaacaatgacatttttcacagaaatacaaaagcaaccgtaaaattcttatggagccaaaaacaaacctgaatagacaaagcaatccggagcaaaacgaataaacaaacaaagctggaggtatcacactgcctgaccttaaaatatattacaaggttatagtaatcaaaactgtatggtattgatataaaaacaaacacattggcaatgggaaagaatagaggacccagatataaaaccctatatacaggcaactggttttcaacaaaggcgccaagaacatacatgagggaaaagacaccctcttcaataaattgtgctggaaaaattggatatctatatgcagaacaatgaaacttgacccttatctctcaccatatacaaaatcagtgcaagacggattaacaacttaaaggcaagactcgaaactataaatctactagaagtaaacagagggaaaatacttcaggaaattggtctaggcaaagattttatggctaagacctcaaaagcacagacaactaaaacaaaaatagacaaatgggactattttgagctaaaaagcttctggacagcaaaaaaacaatcaacagagtaaagacattgcctgtttaatgggagaaaatatttgtaaactattcattcaacatgggactaatatccatatatgaggaactcaaacaactcaacagtacataaaaaaatgaataagtaagtaaataaataaataaataattcatctcctttgcccactttttaaggaggatatgagtagacatttctcaaaagacaacatacgaatggccaacaggtatatgaaaaaaactgctcaacatcactaaccatcaggaaaatgcaaatcaaacacaatgagatattatcttacccagttaaaatgactattatttaaaaaaatagattctggcaaggatgtgaataaaaggggatttaaattagtacagtcactatggaaaacaatatggaaatatttcaaaaaactaaaaatagaactaccatgtgatccagcaatcccactcctgggtatgtacccaaagggaaaataaatattaaaaggatatcttcaccctacgtttactgcagtactgttcacaatagcaaagatatgaaatcaacctgtgtccatcaatggacaaataaagaaaaaatatttttaaatatatataacatgtatgttatatatatatatatatatatatatatccaatcacatgtatgtactataatgtatccaataacatgtatgttatatatatatatataatccataaaacatatacatacatacacaacagaatattatccaggcataaaaaaagaataaaatcatgttatttgcagcaacacggatagaacaggaggttctaagtgagataagccaggtaaagaaagtgagaaaagccttaagtgagataagccaggtaaagaaagacaagtattggacactctcactcatatgtgggagtgaaaaatgttaatctcatggaggtagaaagtagaacaaaagatatcagaggctgcgtagggtgggtggtggagggggaggtgatgaggagagattgattaatggggacaagcattagatagaagaaatacgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnattttcatattcaaaatagatggcagccaaaccaggccaagggtcgaggcagaaatcttgcattatgtgtgttagaatgctacaacatttgacacaatagcctgcagacaggggtgctcaggagacaggttttgagcttcatgcatcaattagctggtctttgagggtgacatctgttggtacatataatcactcttcttcccaggtgtagattgtgtaactgaaggatagggtctacatatgccacgttcaaagtgatttaaaaaaatagtatggctgggctcagtggatcagtggatcacacctgtaatcccagtactttgggaggccaaggtgggcagatcacgaggtcaggagatcgagaccatcctggctaacatggagaaaccccgtctctactaaaaaaaaaaaaaaaatacaaaaaattagccagacttggtggtatgtgcctatagtcccagttacttgggaggctgaggcaggagaattgcttgaacccaggaggcggaggttgcagtgagctgagattgtgccactgcactccagcctgggcgacagagcaagactctgtctcaaaaaaaaaaaaaaaaaaaaatagtatggccaggcacagtggctcacacttgtaatcccagcactttgggaggccaaggcgggcagatcacttgaggtcaggagttcaagaccaggctggccaacatggtgaaaccccatctctactaaaaatacaaaaaattagccgggcgtggtggcatgcaactctagtcccagttacttgagaggctgagatgggagaattgcttgaacccaggaggtggatgttgcagtgagctgagactttgtctcaataaataaataaatgaataaataataaaaatttaaaaaatagtatatagtgaagagtagagtgcctgatttactatttagtaataaaacattcactttccttatagactgttttgaatttatttaggaacttgcccccaaataagtgaacaccaatctagtatcattattgtaccatttcacttgacagttgttacagagtctgttctttaaagtggaatcttgatagaatgggaatttcagactcattttggagataagctgtaaagaactaatggtggatgatgttctcctcagggtaaggtggaagacggggcaattagaccccgtggtgaatgcagattaatgaatctaagtgagtctatttggttttttcagtttagttggccggagtggaggagcaagtcatagaaaattggggcttttttttcttgtttcttcatgccatgccattcagtactgactgagggtcaagcactgtgctaagtacttaacatgatctcttcaattagttggtcttaacttaaataactcagttaatctgacttccattttctgatgtgaaaactgagacttagggtaatttgcccagagtcagttgttttgtaagcggcagaagatggaattccaacccacgaaggctgactctacatcttgtgccctttctccaccctccatagccaatctgttggccttttttctctttctgttgccatcactctggtctaagccgccttagtgttgaggctggtctcaagtggtagtcccctaattagtctccatgggtccactctgctctctttagttccttctccacaaaagagtgattttttttttctttttaaaagataggatttctctctgtcacccaggctggagtgcaatggaacaatcatagctcactgcagcctggaatccctgcactcaagtgatcctcccacctgggccccccaaagtgctaggaatacaggtgtgagccattgcacctggccttgatttttaaaaaccaagtccaatcttgttacacttggagatgctttaccctagggtcatactgtagctcttatttatgacctacagggacctcccttagctacctgctggctaggtcggcagcttcatcttatgctattctgtgttactgtgctgcctgggaatatttataaaatgcaaattcttgggctctgcttctggaaagtctgattgactagatctgagactgggctctgggaatatgaatttttgataagccagattcgggaaagtcttgctctagaaagtgtgttctgtgaatattagttgctacttcgccatggaagctgctcttgtcttttgttctttcactcacctttgaattctcaagagttacagctctgctcatgagagatgctgtgccagagctaagccttgtcaacaaaacgttttccctggattgatcattatggctttggttaacttcttgtgtgtaactgagaaactgctcagccaagagagacggagagacacacggagagagacagagacagggacagagacagagagagagagacagagacagagagacagagaccgagagagacaaacagagatagacagagagacagagatagacagagacagagacagacagagacagagagagagagacagacagaaacagagagacagacagataaagagagaaagacagagagacagagacagagagagagagagaaagacagagagaaagacagagagacagagacagacaggaaagatacagaaagagagagacagagagagagaaagagagagagacagggagagaaagaaaaagagagagagaaagagaggagagagagaaggagagagagagactgagactgagtttgtctccaattggggttaacagaatttttggtggaattaacaccactttttatttcttacaagaactttctctatttaattttctctatttgaaaaaacttgagatgtaaaatgcagatgttcatacccaggtatgttgctgaataaaaactctgtatattggccaggtgcagtggctcgcgcctgtaatcccagcactttgggaggccgaggcgggcggatcacgaggtcaggagatcgagaccatcctggctaacatggtgaaacccctaaaaatacaaaaaattagccgggcatggtgacgagtgcctgtagtcccagctactcaggaggctgaggcaggagaatggcgtgaacccgggaggcggagcttgcagtgagtctagatcgcgccactgcactccagcctgggtgacagagcgagactccgtctcaaaaaaaaataaataaataaataaaaaactcgtatattaaatgtaatcttattcaatatatactgtactcataagcctaaaccccagggtttttcttcctttatatattaacatgtgttttgaaatgtgcttttgaaatacaaggggttgtaaaacattgaacctgcagaatgtggacaataaagtaaccatttttgtacttatcaaagtttcaaacaaccctagaaaaatttggaaaacttttgcaagaatcttgtctgtaaggtttatattctatattaagaagttattatgtaatgcacatacagatacagataaaattacttttcacagattaatttttacagatatttttatctgcctttatcaccaagtatttgatttgtaagttgtttaggtgcacctcatatgtgtgcataaaatgaacaaggtactattcatgtcacatccatttcagttctttgttgttggttaattctagcatttgtactgcattaaattattttattgcagtgttgatttttattttccccatttgcactgaagagaatataaggttgacatttctttttggcatgtttctaagctcagcaaatatagcaaccgaggtgctaaatttcagtcaaaaatagaactcctcagcctcattttggaaatttcttctcattcatgcacagggattctagtgtcaaatttttagctctgccctctgctggtcatttcaaattatgcagaagaattctcagaacagtattcatttccaaatataaggagttacaggaaggcgtcttaaaatttcaccactgcaaaatatattcacaggaagattaattgtaatagctttacattttaaaatcaaaaattctttgaggtaaatttttgaaattagagagtagcgaaagctttatagatttttcaagaaaatagcatttgtgttttttttcccctgagtacaggtgaaaacaaatatggttatagttaaggtatcaataacattagagaaaacaatctgatatgtataaatgctaatgttgattaagtattttttgtcctcttctgatacagcacttactccattaaagtctcttgattggttaaaataacacaacttccttttcagataaaacagatttttaattaagttgtatttgctaaaggttatatgctcctttagctaaatgattaacccttattgaaatatgttttcacttttaaggatttgaacaaaaattttgtacagaagcttacatcaagatacaaagatcatggatgcttatagatacgatgaactgcaaatgaaaattgaggtcagccagccatcagacatagtcaacaaatcacgcgggtggaaactgggtagtctcttctgtgtacagaaggaaaatgattattgatgaaacaagacatccatcccatgtgctcattaattatgaacaagaaacaatgtctgtaaacacaaggaaagtttttacaacgcgagaccaagaaaaacagggaaggcatatcttgctcacagccttgttagatcaaggctttaattaaggtcaagctgatatttaataataatttgagactaaagtccagctaaagagtactgagctaggtgataaaaattggttttgtttctcaactcttaccaacggtgtggcctcaccagtaaaagtcaaactaaatccctctagtgacccgtttaatgctaatagtcaccactttttgagctttgctgagggttttacacatgtcatttgatctttaacaccccttggaggttttgttaggtgtattttttataaagagactggccataataattagtagagaagcattgagatctaggtatgataaactctgaaatgcctgctcttaactgctgtgtctattatgcagaagtccctaaatgtattttggaataatcattacaggctctacatccacacatgttagcatttcacaggtatgttcaaaggcaaagagatgataggttttcacagtaaccttccctatatccatagcttcagaaaataagaaatatatggtagaaattaaccatccttctatgggtctatacatggatattatggataaatatcctggtttctaagtcccaattgttatgttgatttaagggcacggtctttagaaatgacttgttcaaattctctactttattctattgaagtatatttgggctcagtagatcaaagcactgcacgtgaaggcctacctcatttttaaatgatgataataataactgtagccataatggctatatagtctactaatgggtgagtcagtgcccattctaagcaccaacaagtcaccctagtctataagtagcagagcatgtcacagatcttaatctcagttccagcctgtttcctgtgctatccgtggttggtgacagatcttaaggctgagcccttattttagcttttctttctgtgaaaagagacccaacaggcagctttcatatatgtgctacttctgcttggtatgcgctttcctggggcgaattgttcaaacctcaccttcagctaacacctttgagaaaatgtaagctccggtgtgggcgaagtctggattctgagagacaggagtcttagaaaattgtgaatgcagtttcgtacagtatttctttttgcttaagaaaatctttatgataggcatttagatgttgccccactcagcttgagacttgctgtctaattttaagactgaatcactgaaaagtgtaagtctacctctgaaggagcagaactctctgtgtggcaaagcagaggggccaatggactttttctttattttatttttttgagatagggtcttgctctgttgtccagactggagtgcaatgctgcgatcatagctcactgcagccacaaactcctaggttcaagcgatcctcccaccttaacctccctagtagctgggactgcagatgtgcaccacctagccaatctaattttttttctttaaatagagatggggttttgccatgttgcccaggctgctctcgaactcctgggctcaagcaatcctcctgcttcagctggcctcccaaagtgctgggattacaggcgtgaaccaccatgcccggcctccaatggactttttaatgcatgtcactgttctaaattaaaccctgctatcagcacttttcatattcccttttcttattccttcttgagggtaggtaccaccatgagctctggcatttacagaatacacataattctcctctctgtctaatccagtcctgatcctctgtcttctctaggccctgtctagaatctgtgtgctcttcactggacccttttgatactccgagctcatttaaaaaaaaaaaagaaaaagccttacattccttttattccttcccttaactcaaaattggtttccttttataaggctatagtttttgaaattcaccccactctaattccttcctttggatcttctgctctcatagagccaggaggaggatgtgtgtgtatgtgggggtgggttccatatgcttaaccttttgtttccgtttccaaatttgtcctagcagtagcatcatgcggtagactcatacataccacgctctcttcttcttacgcttgttcccataattgctggagaacctcagcacagtggccctgttgcctgtgcctacttctgggatgagctaggactaagcagagacaacatcccgagaaactaatgtcatgctagaataccaatctatgaaattctatttctggaacctttgctgctctactaagcagatccatgcagagagaagcctgccactgtcttggcatttctcagcctttcaaccctgtggagtgtctattttgcaagtcaccacctccttctcccttactcagagccatcagtgattattggagacagctacaaaacagtggcaataaaatccactaaactttccagaggcccagtctgagttgttcaccctgtagccctgtgattttgggtgagttacttatttgagacttagtgttttcatctgtaagatgaggaaaatagaagagttttggtgaggatcagataaaatatcaggcacttatgtaagagaacattatagagtaagtgtggtggaatccccaggcgggtctgagtcatggctttgctgagtgattgctgtgtggcactggctggctcatttgtcctccgtgggcttggttcctagttgttaagatggagggttagtgatatttacaacgtgggctgtaaaggggctatggcaatgcccagcatagaacgagggctgagtagatgctagagccattttatcatcatttttgttgttatattcatggcaggactttttcgggcaaaattcttatatcatctcatttaatctcactcggttctcaaaaagtacccattgagctggttgtatttacagtttttttttttttttttttgacaggcttgcagttgtacgatcatagctcactgcagccttaacctcctgggctgaagtgatctcccacctaaacctctctagtagctggtaccacaggtgcatgccaccatgcttggcaaatttatttatttatttttttgtagagacgaggtctcactatgttgtccaggctggccttgaactcagggctcaagtgatcctctcagctcgaattcccaaagtgttgggattacaagtgtgagccactgctcccagcctgtatgatgctattttataggccagggactaaaggttctcacaggtaaatttctagaaaaagatggagttatgatttaagcccagaagttgcctcatttaaatttcatgaactttttgttggattatgataaagctaacatgcagtaaggggttcaagaagtgtgaacaccagccgggcgctgtggctcacacatgtaatcctagcatttgggaggccaaggagggtggatcacctgagttcaggggttcgagaccagcctggccaacatggcacaaccctgcctctactgaaaatacaaaaatcagccaggtgtggtggtgcatgcctgtaatcacagctactccggaggcaggagaatctcttgaacccaggaggcggaggttgcagtgagccgagatcaggccactgcactccagcctgggcgacaaagtgagattccatcaaaaaaaaaaaaaaaaaaaaaaaagtgttactacctacaaggcctacccagttaagaataattcatgtccgtgtcttaggagaacgtacgttctcactggagagagggagcagacagacaatgcatcaaagagaattctcagttcaaacacagtcaatgctgacacagatagtacagagtgctctgggaacacagatgaagcaataaacacccgtgtaagtggaggaaggccccattctagaaaggtggcatttgaagtggcacatgaaggttaagtaggagttttcaaggtggaaaagacaggttgttcttggcggaaaacctggtttctgcctcagagatacggttggctgtgaaagatcctgacacattgaagaaatggtgagcaattccagttggcagaaaccaaggtgggctgtgaaggatgggcggagacagggatgggtgatacctgttactaagggcatcttcataggcacatccgcaatcccaattccatctcagaatcattagaggtctcaaatatttgtatttactcatttttaaaggctagtcaagtgtagtagtgagaagaaggaaagagtagaacaaggaatttgatctgtaactgattctgaacaatcaattgaaatcaattgaaataactcactacccttggaccaaccagagatctcagatttgtagtagcaagaaaggaatatgctaattttttccttccttccttccttccttccttccctccctccctccctctttctttcttccctcccctcccttcccctcccctccttttcttttcaacagggccttgtgctgtcgtccaggctggagtgcagtgttgtgatcatggctcactgcagcctcaacctttcagacttaagcaatcccccccccgaccttgacctcctgagtagctaggactagagttgcacaccaccacaccctgctaatttttgtatttcttgtaaagacagggtttcatcatgttgcccaggctagtctcaaactcctgagctcaagcaatccacccaccttggctgccaaaatgctgggattacaggtgtgagcaaccacacctggccactaatctttgctttcgatgaagatgattttctgcatagtggagagctagctaccaggatgtggtaatcatgaaagagactataaatcttgttttaatttttatttttttaaccaatctcattctgctgcccaggctggagtacagtggtgcaatcatggcttactgtagcttctaactcctagcctcgagtgattctcccacttccacctccatctctggaatagctgggattagatgtgcactgccatgcctagctatttttaaatattttgtagagacagggtctagctatgttgccaggttggtctcaaactcctggcctcaagtgttcctcctgccttggcctctcaaagtgctgggattatgtgagccactgtgcctggcctatcaaaatgtttttaaactagatttgagagcccaaagtgattgagatgaataagaaacacattctacagacatatctgagatggaaataattcattttggtgacttactagatttagaaggtgtgaggttgtcaaagatcaaggtttctagttttgcagattagatgggcaattctttattgagtaatgttttttaataattcatgctcttaatttttgatgagcaatgcacaatttgttagtatataaaaggactctttagctatatcattaaaaaaaaaactgaccatctggactggcacttcaaaattagatacttttttttttttttaataatgactaaaacttggaatttttataaatatatatcaccgtaaagggacagacgtggtggctcactcctgtaatcctagcactttaggagcctgaggcaggagaatcccttgagcctaggagtttgaaaccacctgaggcaacatagtcagactctgtctctactaaacaacaacaacaacaacaaaaagtcagccaggcacagtggtgcatacctatagtcccagctactaggaaggctgaagcgggaggatcacttgagccaggagtaggaggccgaagtgtgctatgattgcaccactgcactctagcctgagcaacagagtaagaccctgtcaacaacaacaacagcaagaagtatgtcattgaaaacagcctcatgcagagggaaatgttcagctgggtttgaggcagataaaaaaaatccatttgtttaaactccaaaccattcactacttccgaaaatttattttaggatacttaaaatttattgattgccttctcttaagatgtctcggcagaggcatttccttttaattcatccatgttgctcttgttacatttccaaaggaagtgctacagaggtaatgcagagtcaggaaactggttccagtaacgctgtgaagggagtgagggcagggtttagtctctgaaatagactgtgcacttgcttcacaagagggattttttctctccaacagctgagttagagcaggctaaggtggtgggctgaaatcctagttggccctaggatggtaagagaaagcatccactcttgtgatgggacggaattatgcaaagtagaaaaggtttgtcaggacctgtctccagaaaggatcaagcagaatgcattgaccaagaagatggaatttgaacatctacaggttagtaggaatcactgaggaagtgggatatgttagattaatagatgcaattcattaatcgcctgaattataatatacaggaaagacaagaagaaagaaaaataagatctagaaacaaagaggatttacgttgctatttgagcaagcttttgaaagctttaggaagagtggacagaataatagaattattcaatttaaatggatttactcttaattgtacttgctgatatctagaaaatgaaagttaagaaaaaaactttatttcttagcaataaaaggcatttaactattgactttaaaaagtctaccgcacagcttacattatttaatatgacaaattattgtttcctctacagtgctgtgttaatagctaatactttatatagctaccttctgaagctgtagagaggcttataactttaacgtttcatgctgacatttagttgatcctagtttatctggtataaagtagttgctcacaattaattactcaagaactgagcttcgtgtatgtctgtggtagaaatctgtgggagtatgtgagttcattaaaaccgtgctttcctagtcattgctattagtttaacgctttccttgaaatatcagtcactgagagaaataatagcttctgttatggctctgcacagctgtactgaaagattttgttttcacttttcagaagtattagaaaagttgcctccatgtctgggttatctgaaggtgaaaccagtcaatgattataataacttcttttttaatttagaagggaaataaaggaaggaaggggagaaaaggaagtaatgaagtgggggaaaggaaggaatgaaggaaggaaagaagcaatattaactatagtaactgtagttggtactatttatggaacacttgatatttcaggcattatgatttgcaaaaattatctcataatatccccattttacagagggaaaattgaggttcagggagattaactattcaagactggcacttcaaaattagatactttttttaaaaaaatgactaaaacttggaatttttataaatatatatcactgaaaagggccagacgtggtggctcactcctgtaatcctagcactttaggagcctgaggcaggagaatcacttgagcctaggagtttgaaaccagctgaggcaacatagtcagactctgtctctactaaaaaaaaaaaaaaaaaaaagtcagccaggcacagtggtacacacctatagtcccagctacttggaaggctgaggccttccaggatttaaaattaattttttaaattctaagagcatcccctaatccccataagatcctacctctctcatttttttttcttgcagctggataatgttagaagatgtgttaaatatttaatccagccggaatgccaagttagattcagcttggatttgaaaaacaaaaccaaaccaaaaagaaaaccactggttttcaaggcctcaaagcataagctttgtgtattacaggaaagcttctatgcttggctggaggtaggagaacctttctgtctttttctgaatttgctatgaccttgggtaagtcaactgacctccctgggtgatgttcagagcattattgatgttatataatttattcttatcaatgccacaaggttggctagatatgtttcctctctaatgtatgattaggaaagagtcatttagacttcagttaattttattttcttcaaggtcaaccaataattcagacatcaagttgaatggaaatgagcttctaaatataggtcaaatacttttataaaaatcacataactgcatctcccccccaccacgcctgaatttgatccatctcccaataagctctcccaagctactacttagtttctattgttagtatgttactgaccaagtgcaccagtcctgcattttgtgtcgaactgattaataattttcttctgaacccctaatgcatcccctattttgtccacactctcataaagactctagttgataaaaatgacaggctgaatattgctaatgtaatatttcaattcagatgtaacatctcttaaagattgagctaattaataccatgtttttatgtttcatattgtatttattattctttggtagcttaatgattacccttctacataatatttatatatagtcttttatctggacccaataagaagatttgagaaaagttataaaactgctataaaacattataaaaactgtttcccgttcaacctgactgctttgtttcccttaattctgggattttccatcatttgcattatctaattgatgatagaatagtctcatggtaccataaagtaaaagactaacaagaaattttaattggtttgcttgtccaactgctacaaaattataaagactgcctttttctgtgttttttttttaaatccaccctatgaaacttatctttatgtctgtcttttctctgatacgctggggtttaaagtgagctgctttagcctcattagaaataaaaggtatcgtgttaatgtcttctagatgacctttagtaatttgagagatattctccatttttgtaggcttgacactctattttttccctgctcttctaactcctgtattctgtggcttctgtatttgacagtagtagccccactcccagcaaacctgagccgtcacatctctctcttttggttatggagttcaaaatggggttcctcacccatctaagagtcaggtattactttgttcttgctgtgtgagaagtggaaggctagtgaaatgaactaataggatcttcaaagtgaaacagaccatatccaacttgtagccatgtcggaaagcctgtccacactatcattacaagctcagaacagagtgtggggagaacctattctatttaacctaacctgtttgtggggagccagccctgactgccatttattacttttaattctaattacaaataatgcatgctcactgagagggtttgaaagtttattttaaaaagcccaagtcatggtggtatgaatgtttttgctctctcttgagtgcttagattttcaatccctcttggagcatatgaattatttgcatacatggagagatatatatatatatctgtgtatatatatatatatatatatatatatttgtgtatatatatatatctatatctgtgtatatatatatatacacacacacacacacacacacacatacacacatattcttttggtcttcagtttgttcctcatcttagaattttttttttgttattaataaaataagagtgttaacaaaacctctggactcaggtagtttcattggtgaattctaccaaacatttaaagaagaaataaaaccagtcttagtataaactctttcagaaaacagaggaggaatgaacactgcccagcttattttatgaggccaaaatttccctgatactagaaccagatacagtcattagaaaactatagctactatccctgataaatgtaaatatttattaatgaaatatttgcaaatttaatccagcaatgtatataaaggaatgcaaggctggtttaatatttgaaaaccaaatgacatatcaaaaccaggttaggttgattccaggaaagtaaggtcagcttcacatttaaaagccacatatttttcattatatacattgtatgtatatatacattatatataatgtatataatatatgtatataatgtgtatatatataatgattctccctagagattatcagaaaagtgtttgataaactgtatcaccattcatgacagaaactcttaggaaacttagaaatagaaaacttgcttaccctgataaaggacaattataagaaatctacagcaaatatcattagatggtgaaatattgaaggtttgttcttttaagtttgggaatgagaaaacaatgccactattaccactctgtcttgtactgaacaattattcctatatggtctagaagataaataagaaattggagaagaaataaaactgccattatttatgatgatgtgattgtatatgtaaaaatttcaaatacatttacagataaataattagaaataaaatttacataattactgaacacaaaatcaatatgtaaaaccaatgccatttctgtatcagcaaacaaaaatttaaaaatccttaaaaatatacatttaagaagattnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttaaaaagaagcaaatgcatgttatataaaagcgatttggtaaactctaagacatacgtgttccagttgattttaagaaaggcacactagccgggcgcggtggctcacgcctgtcatcccagcactttgggaggccgaggcaggcggatcacgaggtcaggagatcgagaccatcctggctaacacggtgaaaccccgtctctactaaaaatacaaaaaattagccgggcgtggtggcgggcacctgtagtcccagttactcgggaggctgaggcaggagaatggcgtgaacccgggaggcggagcttgcagtgagccgagatcggagcttgcagtgaggcgagatccagcctgggcgacagagcgagactccgtctcaaaaaaaaaaaaaaaaaaaaaaaaaaacggcaaggcacactaaaataaagaaaatgatacctacctagcctacaagattagccacacttatgatccaacccaatcaatggttagattcttctcttagaatagaagctgttaaacaagaggagtagcgccatagccttttctaaatacagtcaagttgaatagagtaattaaatgtcccagcagcgtgacacgcataaatcacatctgtaaaaataataactattatgtattgagtcacagaaatgaatgatgttcagtatgaaaaatggctgaaagaccaaagccagagaggcaggctgggatttcctgtcagctaagtccgactccaaagttcacttgtcctctgctgtaggcagtccctgagagccatgtgcacacacacctctgccataacacaacactaaatacagcacaggaatttgctaacaaaaaaccaagggaacttcttaggctttttgatatttttctttaaagggataagtttaaaagaattagaaactcaatttcttatttatattaacttttcctttcttagaaaaagctgaaacatttaggatgaccttgacaaaaaaaaaaaaaatacaattaaaaaaaaaatccagagttgaaaatacaccaaagtgattccaaacaaaccgatgaagaaacaaaatggcattaggtatatattttattaaagtttttattctagcaggttgcatttctaaatttcacagtttgtttaaaaatatcacagtaacatgcactataatgctgcagtatcagcaggctcttaaaacaaaatgcctttctaatttaaaccaactatcctaagtctccactctttaaagcaaaaagaaaatatgcacagatgtttaaactccttgaaattaaaataacattacaatcttttattctttgaacatatctttttgccagtctagccaattttggcatgaaaaacatttgcaagatataatttggacctttgctatttcaaatatcttggaaaggagaaaacaatgtttcacctatctggttgcatacaagtctgctgagctatatttttcccttcagaattccctctaaaaatctggttttagatatgtatcctatctataattttcatattatgttttcagcacttagaattcagttagaagcatcaaagtttacaataaaaattaaaatataagaaaaccaatacattgtgagtaccatatctaaatgtttcatgatattgccaaaaatatttggatatcaagtatgtgtctacaaaatacaggcatcagcatctcactttgtacttcgttacctcaatgagagatattaatcatcacaaataaaagacgtgatgcagataataaggttaatgacaaccacaaaggaataaaagactcttattgagaccatgtgcatatttattattatgttgtagcaagtatgtattcagagaacattatttggtgcataataaagggctttagtttattcttccttgtaatcactgccaaaatccagtgaaacaggtaccactacggtccacattttacagcagaagtggctgttcaaggtcacatatccataaagtggcagagctaggaaatgagccaggcagtctggttccaggctctacccccttgaccactattccacactacccctcagccatatggagagcacactcctaccaaggaatgagacacacgatgcctcattttccccactatttccttaacgccctgtgttatcaaatataattaatatgggcagacggaacataaagagaataaattagttttcaaattgttaactagaaagcaaaccttagacggtaaggtccatctgaatattctgtcttatgtattgctcaatttcagggaaattatgaaaaacaaataacaaccaaaatagttcccttcctttgtagttataacaacaacaaaatgcctatgagaaatttagatgaaaagtttgatagagaaaatgttaaatattcagaaaacaaatgaatcactgagcatatgtttattgactagaatcacataataaacttttaatgatttagtaaatggtcattcggtattcaggcgttttgcttcacttcctgctctttggccaatggcatgatgtttctttctggcccttttatagggacaagatttgatttcttgctccatccatagagtatgctggtgcttttagcactgtgaagacacaaagatgaaaactggatttgaccctatcctatggaggaagacatgtccagcttccttgaaagtctacaatctttattcctagaaatgacatactgaataagcaagtggctgattcttcaaagacctcctagagtttggaagttaaagagagataaacagtggcatgtcagtgagttaatttatgatgctcattttcagagaataaaaatttcaacatgtgattttaagcctttacagaccaaatagtcttctttctgatggcatcattttcagagatcatatatttagcatttttcctttctttaacaaatccatttgaagaaatttatcatattgtgtcaaacaattctatcacatcctcgtccaacaatagccaaatcattcattattcctatccctgaagacatcttcaactctattgtcccagagtctcaaattagcgttttgctgttcaataaaaagtttttctaaagtggccactattaagaaattttagtttttaaaatattgttattctatagtaaagtgaacgaaactcaagtatagagttcatttaattattgcttacaaatgcatcattgtaactatcactcagatcattgtataggacattatcagcactcctgaaggctccatttatacctccttcaatactcccccacacaaaagtctccttctaacttctatcgtcacagataacttctgcctgttcttgagcatcataaatggaaacactttgtttataatggcttttgcctgtaaaatccatattgttgttgagtataacagattgctatttttcattgttactattccaccgtgtatctaatattgatagatatttagaaggtttgcagtttagggtttttcaagtaatgctactcctatgactactgggtcctagggtaaatgtatttgaacctgagtatttactatgaagtagatttccaacgtaattgtacttgtttgtaactctagcagagcaactaatgaaatgaacaaataaaaacttcatactttccaataattttcttaaaaatttatcttgttatgataaaattattagctacctcagaacaaaatcacaagattaaataagtcacgcaggagcctataaactagttaaatatcttgaaaagtcattatatgtgtcagtggtttctaataccacatctacagatacatacacatgatgaagagatacactgttaggtattcaccaaataagttttaatgtctttacccaagatcactggctgaacattcagaagcagtggttataaggatacacaatgttaaacatatttcaagttttcataaatatctctattgaatatctaatagatagctaaatgatgtcattctaactcctaaatgttcctcgaatcagtcctttcttctcaatcatcattaccttattttaacaccattatcttccccaaagcactacagtcagctgcataaaggtatttagttaaaagtaaatttgaattttcgataagtatacaagcatttgctggaaatgactacaggtgaatgtttatttctgggttggatttttccagctagttcattctgcttaatgcctagaatctgtagacaatgtgttcatagaaacaaacacagccagatcaaaaccctgaggaaggaaatcatcttttaaaagactcattgagaagtcaaataaaagcaattttagaatcagatgctatttgggattatgaaagtttgttttcccaagcctagtgtaattctcttaaatttaaggcagcaagtaggcaaagaaactcaagtatcctctagaattctgcatgtataactcaactccaatctctctcaaagcccagtcttattctgcaatttgccccctaccctgcccacccctcccaagataaagtactaagcccagtgcttggcacacagttattcttcaataaacagtaatggagtgaattaatgccacatggagtctctgattatcttaatgctccaatgaaattcccttctgtcaccacaccatgactcaatctgagaggaagacgtgtcccattctgtaagtgattccatcccaagataaaaagaccagctgcggtatttaattagtctatttcgaaaaatccagagaagggaaagatactgaaaatgcatatcagtaaacaaaatgcataacagcagttctacagggtaggaaactgataacggacaagaccttgacacttaaaatcacatggtctgtgcaaaggggcaagctggggtcaccagcagctggtatgcagagctgagtcaaggctgacttgaacaactgtaagcagctcttaaaaaaagatccctgactgttttctaagttggtggcaacttatccgactatacctgttaattatccgctcaagaaccatcaacagggacagtcagcaatcaaacaaactcttagaaagacagaactgttagcccagtctttaaatcattgttttgtttcatgagataagaggtagaaatgtggcttaagaaactaagatccaaatcctagcctactccatgactattcccagctatgtgaaaaacaacaacaaaaactcacatccttcctgtccttagctaccattagtggaagcaatattgactttgctgaagacataaagaataagtcacttaactttgtaaaggcacttccataaaaagttaaacccaaagtagcaaaaaggaaaacaaaatccctgaaagaaatccctggaagagtttaatttttatatggctgatatagtttggatgtttgtcccctccaaatctcacactgaaatgtgattctccactgttaaaggtgagtcctagcgagaggtgactgggtcatgggagaggatcgctcatgaatggctttgtgccctcctcatgggaatgagtgagttatcactctattagttcccacgagatgtgattgttaaaaagaggctggcacttcctcctctttctcttgcttcctctctcaccatgtgacatccctgctcccctcttcaccttctgccgtgagtaaaaccttcatggcagaaggtgatgaggcctcctcagaagctgagcagatgtgggtgccttgcttgtgtggtctacagaaccatgagccaaataaacctcttttctttgtaaaatacccagtcccaggtatttctttataacagtgcaaaactaacacaggggtcttttcaaaaagtctaatgattctggaacacataatctttaatgctggcgacgaggaaataccatggggtgtcccactgtattaggggcctgaagtatgtagttaaaggcatggcgagagtgagagtcactaaagctaaaagcttccattctgaacagtatggatggtgctagggagatgagaaaaaattccattttcaggtcacacctgaaaggaaatcacctttctaccaatagaactgaatgagtgcaaagagctgaagtcatgcaaaaaacgccataatgatcagcagaatcctaggcttcaactttcaccgcagatcatgcatggagtaaatttgctcaagcatgaatcacacacccacctcctttgtgtctatctccaaacgggtacaaggtaattccacaccaaaaacactctcctggggaagaagaaaccctcacacatgatacaatagacatgaataacctaagatattaaaaaactagaaacttaaaaaagaaacatctgaatgatcaggagtgactaatgaatacattaccagacattctgggtgggatttttttctgccaggccaactttgatatggggcagaggaagaagaggggagatggctccttattctcaaaggtactggttgacttcaccagaagacaggaaaagcttgaggtttatgcccttcactgatgcaagattacacaactaactaatctggggtctggacacagtggctcatgcctgtaatgccagcaacttgggaggccgaggtgggtggatcacttgagcccaggattttgaaaccagcctgggcaacatagtgagaacccatttctaaaaaaaataataaaaaatttttaattagccaggcatggtgtcatgcacctataatcccagatacttgggaggctgaggcagaagaatcacctaagcccaggagtttgaagctgtcgtgagcgatgatcacaccactgcactccagcctggaaaacagagagagaccctcatctctgaaaaaaaaaacaaaaaattaattaaattatttaaaaatcaaaatctgatccaaataaaaggagcgtttaaaatgaaaagcaaatgaatatcatagcaatattaacttcaccatagactgatatcttggcttagaagtctttctatttaatcgtgcaaagatgacgtccatgcactctttctcaacttgtatatgaggataccctggacagaaagaagtcactcttgatttatcgcaatatcttagacttcatcttttccccctttctcatttttgaattagaagccaaaaaactaaagtagaggcaggaaagagaataattttagtttgtataaataccaaattactctttagtatgagtcatgtgttatagattctttgaagaattttgcatgcaatactggttgccatacacaggatgctaattgtagctcaacatggaatacagaaaagaatacagatccttgaacaatctgaaattgtaagtgtagttaagagggatgtactcattcctcttacaaacattagaagctgacatggccaggatttttaaaagtttataagtatatgaaaaaaatatttttatataattgctttgctaaaacataactggagaaataaagtaaggaaactagcttgtcatgaagagaataaaccttagtttcctcagggatacagggaatacaggaagttatcctcatctgactcaacgtgattcaacatggttttctaattatagctaatctgtggggaggctgatacacacatttgtagtgggggggttggtggtgatgacagaaagttttttacacatctcctaaagcagaaatcatgtggttattgtccaacatcacaattatggcaagcaagatatatgtctctttgtggtggaaaagatacagacatgcaaactcagactggatttgggctaaagagacaaatcagcatcttcacatacaatgctcaggtgcactcacccttccaaggcctgtttccctttcttctacaatgggttttcccttttctggaagatctctacacccttgatatcttctccgacagaagttctgcccattgtgtaaaatttctttggcccttctcacctttttctcctcttttcaggggtcatcgggaagaaggtaagaatcatggcgagggtaggggggatgagtgtcacctgaggaggttgttaatgcaaaaaaattcaataatttcttcgaattttttctgcctctttttcttttccgctttgtccccctagaaaaaccagctggcactatgagctcaagggaagtctgactggaatagatggaaaggaatgaatttaaactcacggctgtctatgagtttaaaataccttatcagttaaaaagatccaagaaataatgaattcatgccctgcaaaaattctatccttccaacatccctttcatccatctgaaaggctgaatcacatctgcctgggactttaaagtaactatgaaatatagaccatcattgccagtgggtatcgtcaatagttgtaaagaatccatttccagggtttctggatcactaaaatgggctattttgcttgttaaggagggagggggccaatgtgcaatgtgtgagtccgcctgcctcttgcttctagttctttttatgcgcatacagaaagaggaggaatatcataggctggttttatccctaatgaattaatgaggtggtcctattctttgtaaggtcctactaattaattaatgaagtggtcctattctttgtaaggtcctactaattaattaatgaggtggtcctattctttgtaagatttatatattgagtttttcagaataagcaacatctttcaaatgtgaatttttctcttaaactcagcatgagtcttcagaacaatagtatgtcacttcctctagatgacttctgtggaattaaatttaaaactcagtagaatagtcttagcaagagagatggttttacatgctttgagaaagaaatttaaaaggaagatagacccatacacagtctttttttttttttttcttttttgagatgaagtctcgctctgtcacccaggctggagtgcagtggtgcgatctcggctcactgcaacttctgttcacctccagggtttgagcgattctcctgcctcagcctcctgagtagctgggattacaggtgcccgccaccaagcccggataatttttgtctttttagtaaagacggggtttcaccatgttggtcaggctggtattgaactcctgagctcaggtgatccacctgccttggcctcccaaagtgctgggattacaggagtgagccaccaagcccggccatcatacacagtagttttctaataatctgaatttattaagtggctgttacagtgaatgaatgaattttttaaaagcgtgggcaacaataaaccactagaaatttacaacagcttacattttaaaactacgatgttgatgtgagtaaacacacagaaaagataaaataaatatctgagcaaaacagcaaggccacagtaagatgagtaagaaaaacagtaaggatgtgacagattcaacaaagtcctgggtggcctagcggggaaaaaaaatgtatagtagactattataaggaaggaatgccgtggttattaaaacagggtactgaaatctgtgaaaccatattagtaacctcaagaatggcaaacatgacttattaagttggattaagaaaaaaagagggcaaactgaaaacaaacaaattactgtttggtatgagtcatatgttatagattctttctagaattttgcatgcaatcctggttgccatacatgagatgctaattgtatctcaacatgaatacagaaaagagtagagattcttgaacaatctaaaatgttaagtgtagttaagagggatgtattctttcctgttagaaacatcagaagctgacatggtctggatttgttaaagtatacgtgtatgaaaaagatatttttaagtaggttttcaactccatagtgttgcataatttcaaattaagaaaaagaggctgagtgcagtggctcacgcctgtaatgccagcactttgggaggccaatgtgggtggatcacgaggtcaggagatcgagaccatcctggccaacattgtgaaaccctgtctctacttaaaaaaaaaaaaaaaaaaaaagattagctgggcctggtggttggtgtgtgcctggtgcctgtagttccagctacttgggaggctgaggcaggagaatcccttgaacccaggaggcggaggttgcagtgagctgagatcgcatcattgcactccagcctggtgacagagcaagactccatctcaaaaaaaattaaaaaagaaaaagaaaaagaatgagcaaatggaagagattcaacaacataaatgttaagatgctcatggtagagaatataagtaatactcttatctgaaaaagtgaagaaaacatatggaagatgttaccaggaaggaaaccaagcaatgaggatcaatgaattcaagaagtgcctacaaacgctttggagaacagagggatttttaaaagggataaactcagaggatgataagaccggatagatgtgacaagaggacttcttcaaaaccactatttctagtgcttgaagacatttattaaataacataaacccaaagaagcttgccttacataagctctttatgctgtttgggtagaaaaaatctcgcaaactaaatccaagtttgaaaatgcaaaactactgaatcagttcaaacctaattattatttcaagtcaccactgtacctactacaagcccctcacaaatcatgccagattaaacacctcaggccctcaaatttctatgccttcaaatcactattcagaagctgtgcttttcctcctagttaaataaatgattctaatttagccgatggtataatttgcttttaaactgtgtattttacactaacacctgtttccttcctgttcaactttctgtcactaaaatattcagaataaactgaaaccaccttgccatttgatcttcttcatctgcattttataatgatcagattttttaatacagtcaagctttgttcacattggtagaaacatagcgaaatacattttcaatcattttaaaaagaaaaagctttgttttggtcttatatttggcaagtttgtattgttttattttttggaaggcttaatgtggaacgcaaagagatggtcaaattagagagttttttgttttgttttcttgtttttggaatagctgtgtgtctacctgcttcgtgtgtggcagtattgagaagaaggctggcattactggaataggaaagtttttacttacaacacaacagccaagtcagtctggcatcactgggggaatactgcaatctagctttcaaaacaaaacaagttactgtgtctttccttgccagttcaaaggatggagaccaactcaaagccaatttctatggagccttgtgccgatctaggccattgaagaaaagctgatcatttaaaaagcactcagctgcaagtggatttcaactcagattctagcttctagcatgaagaacatgctagtttcttttgttggcactagaaaaaagcaggcaaaattccatcctaattatgaaaagctttacttacaggggtaattaagtacacaaaatgttagtaaaaggagctgcaagtaaaaacaccaacaacattatacaggagagtattaaaagagaaaaggagtcattctgtactatatttacagctaaggaaatttaaagagtgacctatacatgttttgtaaaaagttttaatggcaacattacaatccatatagaaaaatacacagaactatacataaagattaacacatggatcaattaaagaaaagccttgcctacattcctactaatacacacctcaaaacaaaatttttttatggctctctgagaaaaaataggaaaaaacagaaatactttaggtggcatcaaagctttaggggacgtcagctcttcctgctctcattttaacttcatttacactattctctttcttttctataagctattgggttagtttttttgttgttgttgttgtttttcagacagggtctgactctcttgcccaggctggagtgcagtggcttgatctcggctcactgcagccttgacctcctgggcttaagcgatcctcctgcctcagcctctcaagcagctgggagtaggtggggctacaggcacatgccaccatgcccaactaatttttgtatttttttgtagagacgtggtctcattgtgtttcccaggctggtctcaaacttctgggttcaagtgatcctgccacctcagcctcccaaaaggctgggattataggcatgaaccactgcgccggcctctcttctataaatttttggttagcaaggtttatactacagtttcttaaaatgtgtttactaaaacttgatatttcaaagatacttttctgctgggcacggtggctcatacctgtaatcctagcactttgggaggctgaggtgagtggattacctgaggtcagcagttcgagaccagcctgaccaacatggtgaaaccctgtctctactaaaatacaaaaattagctgggcatggtggtgcatgcctgtaatcccagctacccaggaggctgaggcaggagaatcgctggaaccggggaggcagaggctgcagtgagctgagatcatgccactggcactccagtctgagactctgtctcttaaaaaaataaaaataaaaaaaaagtcagctatggaaggatagcacagacatttcattcacttttaaacctggcgcatcatcacatgaaacaattccacagaaccaccaaacatagtcattttttataaacagtacattggtactatcatagcagcatattaagtcctgactcttctagggaggcatcattaactgtcaccaataaatcaatactttttgatcttcactctgtatatgagcataaagggtaaggtatgttgattaccaagagaagatcaaacatcacagcacgtgaaagaaagagagagggagggagatggataaggtgaggaagagggggaagaatggtggggtatgggctaattttaatttgtgcattcaaatttaggttcaataatgcaaatctctatttccacataggtctgttcaacgtgtaagttaaaaacacagaagtctttagattttgctttcaatcctccttcctccatgaagacttgatggaagaaagagtcttcaaaacttttatgaaaataggcaataaaaccagtttactgtcaaatattaaaacaacagtggaggtaatcttcataaacacagcaaatctgtctctgggggattctggtgactgtgtccgaaagtcaccagaatcccccagaaggccagaaaggattttccaaaggggttttaattatattcccagagcccagactcctcaagagaaaaatcacagtccatatatctttagtctcttcatcaaaagcaaagaaaaaagttataaatttctccttctgagattaatgaactcttgcctataaaactatattatgctttaagttaaaaacagcttaaacttttgaccatttatttttagggaaaaaaaagtttatcttaaggttttttgaggttgaaaaatatattatttatccagaaaattatagtctgatatcatgatcattctatgggtaacaagtgctaccagcaaaattatttaccttctcataaaaaatacttactagcaaatacttgcataaataggctaagacttgaaattcttattgaaattcaaatggctgcaccttttttcatttccaccagcggtgtatgagggttccagtttctccgcatccttatcatgctagctatggcctttctctttgattatagccatccttgcacatgtgaggtggtgttgcattgtggttttgattttcatttccctaatgctaatagtgttgaatagtatccttttatgtgcttatttgagagtggtatacttttctggagaaatccctactgaaaccttttgcccattttttttctttctttttctttttctttttctttttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacttggctgtattacttttaacatctctttagaatgggagctgatacgatgggaaggttaaagtaatgatctttagtgaacaaacacagtggaagcaataaacatttagggaaaaaagggagataaacagcaaaaagaaccagcctttttggttcctgctcacatattgagaagtttctttctacttatttgagctagagttgcaggaggtactggaccatactttaaagcaatctatcccttaattgtctcaagtaatgcaaggaaggaaagcactattttattccagcttcactttccttgaatgatctatggatggaaagtgcagctcagttttttacctcctaatcgcctcccttgcattaacacccaactgttctcaaatatcttttttttggtgtttgttttttcaagaaaaatcttcctggccaggcacagtggctcacgcctataatcccagcactttgggaggctgaggtgggtggatcacctgaggtcaggagtttgagaccagcctggccaacatggtgaaaccctgtctctactaataatacaaaaattagccaggcgtggtggtgcatacctgtaatcccagctactctggaggctgaggcagaattgcttgaacctgggaggtacccaggttccttaaagtgcatctgagggtacaggaaaaaacctaaccacaatgctttccctattctctcactcaacaatgaaaacagaggacttctgtgacctctatcatcaaaatgtgcaaagatttcgtccctccagcaagcaatcaattctattatagacaccagatgagtgtcctctaattcaaatcaattctaacactatctacctggaggtagcatcaggtcccacaggttgagggctcagtcctcaaaactgcccccacttcatatgcaagccacaggtccaggcctctaggaacttctgaccaactggctataaatccaggatccctccaccccctccgcaggttcaatgtgttagaccagcccacagaactcagggaaacacataacttttgctgacttactagaagggatgttacaaaggatgcagatgaagagatggatggggagaggcttgggaaaaggggagcaccaccctccgggaaaaccatgtgttcatcagctatctggaagctctccaaatcccatcaattttgggggttttaatggaggtttcatgatgtaggtgcgattaactacatcactgggcactggtgaccaacttaaccttcagcccctcccagttcccgaggctggggggtggggcagaaagtcccaaccctattactatgctttggtctttctgtggaccagccccatcctgaagttaccagacaacaaacagtagcatataaaaagacacttaacctcttcgaagattccaaggattttaggagttgtatgccaggagatggcaattaagaccaaatgtatgtttcataatatcaagcatcttatcctccaacaaaatactcctttgcctgtcctgtagtttacctaggcttgggaacatctttcgatcatttctctaattcattgtctcttaagctcaatgtgcaccagaatcgcctagagggtttgttaaaacacagattatttgggaggtcgaggccggcggttcgcctgaggtcaggagtttgagaccagcctggccaacacagcaaaatattgtctctactaaaaatacaaaaacttagctgggcatggtggcaggtgcctataatcccagctacttgggaggctgaggaaggagaattgcttgaacccaggaggcggaggttgcagtgagccaagatcatgccattgcactccagcctgggcaacaagagcaaaactctgtatcaaaacaaaaacaaacaaaccaaaaaacacacacagattgctgggccccacctccacagtttccagttcagtaggtctgggtagggcctgagaatctgcatatctaaccagctcccagttgatactgaagtctgcaaaccacacagcctaatttataccctagacatggttaccaaacccatcatttcaattcttggttgccacttttcctgtatcacatcagcaccttagctgagaagtaatggaattactttgagagtttaatagctggtcctaactggctccactctccctcttcctatcattctacattcccttgccaggtaattgttccttaaactgtcttgctcattcctcaaaaactcatggctcccgactactgagcataaaacgttttaaatcctctattaggctcataagaccttgaatagtccataaaatgtggagactccatcactcactctgcctactgcaaaaggatattgtgagaatgttgggtttttgtcttgttttggttttttcaagacagggtctcattctgttgtaccagctggagtgcagtggcacaatcatagctcactgcagccttgacctcctgggctcaggtgatcctcttgtctcagcctcccaagtagctcatgccaccatggccagctgttttttttattattattggtagagacagggtctccctatgttgccaatgctagtctcagactcctgggctcaagtgatcctcccacctcagcctcccaaagtgctagaatttcaggcatgagctaccacacccagccttattgtgagaatttaaaaagatacagtcaattcttgttattcacagtagttttgtcctataaagtctttgtgaacatcaaattagtgaatactgaatattgcttctagaaaaaatacaggtttagattcctgcaagcccctggtcacatttttgtcaaccaatcaatgcattaccttttttaaatgtgtgttcctgctgaaagacactttatttaatatatattgctgatttactatggccaacagcagcatcactgaagcctgaacaaagcttatctgacatgtgtttttgccacaaggcacatcacagccttcttgcacgtaggaacattagacaacacggcagctctatgcctggaggcaatttcgaacagtaaaaccaacaagaaaaagcacaaaaacatgaaaaaacagccacaaaaacactcttatttacagaatgagagctgaaagaagaaagcagagcttcacctcattcaacatcagctgggaaagtccacgtcaagcaactcaaatttcttgccacttggagcgtgtttgtgattggccgtgaaagcaccatatgaatatgatttgggggctccaaataaatgttagcaagtaggtgagttcgcaaatacatgcaaatccaagaataatgaggaccacctatgcaatgaaagcaactttcaaaaccagagtattgctcaactataacaacaataatgataaagagctaacattttgagtgccaggaactcctccaaagcattttacataaatatatttaaaactcacaaaaaaaaaatgtagagataggaattcttattatacccattttatagatgagaaaactaaggcacacagagtaaaatgctattcttaaattctaacaaagattctcttcttgatcaagctctagccaggctcctctgagccctcttctcaactaggccttagccttggcctgtaaaggcttgagcaaacattaacatagtttctaccaactaaaggctgcatcccaaagataatcctaccgctctttaaattactgcctaagaaaactcaaggctgccaaaacaacttactgtttcagccttcacctgaggatggggcctctttcctagtctctggcggaggacagactcctaacttataactgccagttagtagacacagctggcctaatctcatttacactaatgaacccttgcagtttttcacttctgagtctcctgagcctccactcacccttctccctattcctttattctccctttaaaatgcttggtcacctctgtacaaatcaaagttgagtttagttcacgctggactcttccctagcgcaatagttgccactgatataaaatctgtccttaccattttaactaatgtctgactttgttcatctttgacagttccccagttaaaagtggaataactgggatttgtgcccaggaaaactggtccaaagcccagatcctcaaccactatggtatgaatataagcaaatgttctaccactcctgaaatgcaccctagactgagtcttctcaagtccagtgtctagaacaaaatcaagtttagacaaatttagattaaaaaatagaagtagatttttaaaaagaggagtgcagaagaaacatgtataagacagatatgaaggatcctaaagattaaaaaaacaacaacaacaacaaaaaacaaacaaaaacaaaacaaaacaaaaaaactagtgtattttttagggttggaacaagccagccaatcatacaggaaatagtgagtgataacgctgaagactgaggatggcaagggaaaaccccatgtgtcaaactcaggagtctgaacattttctcaaatgctatgagcagtcactggatgagtctaagcagatatcagacatcagcaaatttggattttagaaagataactctggaggcattgtggaggagagattagaagggttaagactggaagcagagagacccatcgggagaatactgcagtaatccagataagactgggctctgggttaaggcactgtcaggctgcaggccaaggaggcccgggagaaggaaagcaaaacctgggtctccagggcagctgccagctgagctgcctctcaggtcacctggactattgcgcttctccctggccccacacccagtgccatgttcacttccaattttctacctttactctcttctctgatctgaaatactcactcttcttccctccatctatccaaaaatgacgatttgatctaaatttcacccttcctaagaaattgcccttaattgtagtctctcaacaaccttcaccctttttgttaagtttatcttggcagaattacattgcatctcgcaaatcatttcatgtacgtgaaagaggtatgttcttctaactgtctgcttcttaatacccaagattttgtcttataattcttcgtatttccttcaatatctaccacaaataccttgcacacagtaggtcagcagatgttcaataaaggacaagcctcctttataagcagagaacttttatcagctccacgacccccattggattagctgcttggactgtttgcctttctccacaggccattaataatgttgcatagtgcaaactagttttacaccatctaaaactcaatagagcaaagatattacttcatttcacccccaagcctgaccaccccaaggaaaaacaaaaacaatctgtgacatccaattatttcttcacattctcatctcaagttctaactgctttccagatgttagctggttttcattcttaaaactcatagaagagtttcttttgtgattatttctaagctaaaacgtctatatttttctctgagctccttggagtttctacagaataaaaattattcccacaccaaaaaaaaaaaaatcctttctttctcttaacatagtatggtgaatacctttcaaaatgttacctatcttcaatgtcagcactgccctaagggtatcctgtcttctcttcccttcctaaccatctctcgcttgccccttcccagactcattaggtcagagtatttagtggttaaactatttattgaggacctaagaatgattagacactgaggaagacttcaaggatgactaactcatggtttcagacccaagtatctctctctctatctcctttaagacttgtatttcttactccgccttattttagttatttacttgtcttgtgggttattgttttgttggtttccaattactttgcaaattcatattaccttttgccttggagttgagaaagacctcatactcaacaatgtttctttagaaactttctatatgtctaagacattctaccccaattcaaatggcttgttttattaagtctccagcattatttcttcagaatcacttttactccactttcttttgatctgaccaatgcttcttttgctcttcaaactttgtttctgagtaaccaagaccatccacaaggctactccacattactccatctgtttccttgacttgttcctaaattatttctgaaactaatggaactgagtggccactgcataaagagtactataacagatactttaaggagatccagtaaaaaaatatttacttcttcaaataaatttgcagttagattgggcaatgaggggactcggtagggctgatcagagcaaagcgtttttctgacacttgagaatttatttgcatatagatcatacaacatatgggggaagacagtgctgatatcgtctaattcctttttattgtctagcactttgtaatagatgtgactattgataaatgataaatattataatgttcaaagacctgactttcaattacctgttattttcaattcttacccaaagctagagtttttaaaaatcaaaatgttattctatttcccttccagctagtccatgtatattagaaagataccaagagttttagaaaaatgctttgaaacaaaaattgtatatataatttcaaaagcaaaaagttagaaactaggtggctaccaggtgaagcatgttaccatacaccaatctttaacttagaagcaaatgttcttagataaaataattgacactaagctattatatactacagagtataaaaggctttacagtttaaatatttcaaatggcaaagattttaataaacagaatcaaaagacagtcgcatttcagttttatgacataattcttcagatagagcagattttcagagcatattacttgaaattaaaataatcagtaagtgaggtctcaagaatgtaattttatttgaaaacctaattccacagggtcatcaataaaagcattcaaggagtagctacagtccgttcttttcacttgagtgccagctgtatttgctttaagcatcagactctctggcaacaccacagccttctactcctgtgagttgaggaaaggctttatgagtcatgtgttttttctagggatatcgcactctcagaattattaactacagaaagagtaaaagtcaaaactcattcaataaattccaatgtgctcagattgagagaacaggtgtctgaacacacatctgaagaataacttagagaggatgctttccaactcggctgcaagcctctgactaagcagtcctggggatctcacattacgaccacagaagaacgcaggcaacgctgatggaccagaaaaatctcttaggttcatcattcatactgagaaaactgccttcaaacctctcatttccagctgcatttagagatacagaactgccatctagtgactaagaaggaaaaagcaaggactttccatgaaaaataattttttttacataataacaaagcactaaagttagtaaaattaacacaacttagcacatcaaagttctagtaaaacttcaaaatgtataagtgaactaatatacctatttgcctctataatgtatctggacatccaactgatacatggtagagtttacatataataaaataaatttagaatctcatctcattcatctgacgattttggcatcgggtgattacccatgtaaaagtataatttttatcatagtataagtataaaatcaaacagatctgtactcaaatagcgagcctaccctttatcaagtttgtgatctgatgcaagttattttaactcatataatcattttctttagcagttaaatgcgcattataatagttactttaaaggattattgtgaggatttcttaaggtaatagagtaaaagaacaggtatacagtaggttgacaattaatttgagttttccctatcccctttacggccagttactttagcctgtcatgaaacatatccatatcatctaagaaaagaatcagcccgtaaaataaatgtatgacaaagagttaatgattccaaaagtaagtgatgtcattgtcgggtgcttaaaatcgattgcttccaaatattgttccctctcaattaattttcctgacttccagacacttcactgtcaacaaggataagatgtaacatttattcaatctatttcacccttgagtcttcaagttgtaaaatcaagtgtttttaaataacctactccattaggttttctgcttattgcaatatattttaccatggtccatcgctcaggaaaaacactataatgttacacacatgaatatgtatcttgaaatagcataatatagaaagcaatgctgtacatgttttatcattgttgatattatcaaatacttcctgttcaggttctctttgacattatcgtatgtaacaattgattcatatacgtcctgaacataagaatttagaaaaacttaatgtatccctttaaatcacaatttagccatgctttccacaccaaaaaaaaggcttaaaaagtgagggaagatttgagggtactatctttcttaataactgctgactttactatttctttttacctttttatctttctttctcttattcgctgtgggctaatcattgcagataattaaggaaccatcagttgagcccaccgtgcctctccaagtgccaccaagttgatgtagccctctgctcccataacaataagctggaaaaacttgctttttcaatgaactgtccttaaggatagctaacctagccgcatttttgaaaagcacccaggtgctgttttcaaccaatgttgtaccccaatttctcctccagggggagcacaactttcatgtataacacagggattcttttctaaagggaagttactttatttgatgagttatcagagatcagcaataatttaattttacagagaagaaccctatgtcccaggtaatctaagcttgttgtcatcacactaatcagaaatagatccgggtgtaaaacccaagtatgtgtctcctcattttctcctagaccaccaacagaacagaacacatctttttactgttcatttctaatactaagttactgacaaaagcatagttgggtgaaaatgtaaattggtttgtaagcaatggttttatatccaaaagagcaattgctatccacaattactattccatacacgtttcctttcttgcagttttttttttttaataagaaatccattttagcatagcttgcattttagcgtaagaaaaaaaaacccacaaaatttggcttctttggtaactgatatggtttggctctgtgtccccagccaaatctcatcttgtagctcccataattcccacatgttgtgggagggacctggtgggagatgactgaatcatgggggctgatctttcccatgctcttctcatgatagtgaatgggtctcacgagatctaatggttttaaaaatgggagtttctctgcacaagctcttttttgccttctgccatccacataagatgtgatttgcttctccttgccttccaccatgaacttgtgaggcctccccagccatgtggaagtgtaagtccaataagcctctttcttttgtaaattgcccattcttgggtacacccttatcagcagtgtgaaaacggactaatacagtaacagaaaaaaaatagtttcctggaaattaaacagatactaattaatgtttcatccaccaaagtaataagaaccttatttgactttagcaaagctctgacagatttctttttaaagatcatagccatgggactgtttgagcaggcacatctcgcagacttctccctggagtggccaccgtttcctgctggagcccgctgttattgggcccaactctttggctggatatagaataaaacagcttctgaaaagcttgtactcagttccatttcaaggctgattttgtatgcaaaaaataaatggaagaaaacatccccttctgcctgactggattccactgcaggcttagggaaaacaagggcatcctagccagccgggcagatggaaagataaatcgcagagtctgtcacaatcttaagacaaataagacagatgggaatctgacatcctaataagatcatcttaccttctggacagcatctgagcgtttttggggagtttctgaaacagcagattgagattctagattggaaaggaagcagacagtcagtatcttaaaggacaattttcctatttcttcaatcagaaaataaaactttcactaaaatatatcattaaccataagtaccaaaaaatatccagtctctcttccaattatataactgggttattaggatatgactacttttcaatatatataatacatacaatgagaatgctcatgtcatttttaaaaattgggatattcctatgcccttctctatatatcaactatccttatttgtctcgattgctagtttctaaattgtgccagaaagtccttctaagactttcacagattatattattcacttagctgaagtctgtatcacgatagaagccacttcaccaaggttgaaggatgggagctcaaaagcctcatctaactgttgtcaaattgccccttgatttcaacaagagataacgaacactgtcaccaagagacactgagtaagaagggattctgaagcagtttttctcttttagtcctcatgtcatctgagattctatgagactgcaaattcatcattctaaaaagacctaaggcataaccaagtcagggatttcttttcgtaagcgagagggcactttcttctaagtgaagcttgtgtgcaaatttaaaaataaaagggcctttaggataaaaataaaaatgaaagggccttcaaggaattcctcatgctccatgaatcagagcctgaaaaaccactaatctcatccatcctctgcacctttcagatgagaaattcaaagcccataatgcaagtggttgttggcaaggtgaagacgagagtttatatctcctgactgtcgcgttgcacccctgactggcatgcagaccagaccctggacacaaggtacagcagtcactgtttttggttttgtttttttcttttcttttttttttttttttttttttgagatggagtttcgctctttcacccaggctggagtgcaacggcgcgatcttggctcactgcaacctccgcctcctgggttcaagcgattctcctgcctcagcctcccgaatagctgggattataggcacccgccaccacacccagctaattttttttttttttttttggtacagacggggtttcaccatgttggccaggctggtctcgaactcctgacctcgtgatccgcccgccttggcctcccaaagtgctgggatgacaggcatgagccaccgtgctcggccagcagtcactctttatggtcattttgacatttccaatttagccacatggtagctgatatatgtgacaatgctacagaaaaataaaaggcatttagtatttaaacagcttaacatactgttaagctacaatgaaagttttcatttcactcagatgagaatctgtctccacagggacccacaaggcctggtttcaactactgttactccctagcctctctacccacctccatctgtcccaacttgctcaccaggccagccaccctggccagcttacttgtcttgtcacacaccagtcatgcttcttccatagggccgccaaagccacagtggctactgcctctgcccagagcactgcactcactcttcttcccaaaatctgcaagccccactctgtggctgcttccttcggggctttgctcaagcagcgaccccacccactccttggcactcagcacacactcacccttactttcttcgtaacctggatcatcacaccatatactccataatgcatttggttgcctacttatttatacctcttattccactagaacataaactctgtgaaagcaagaatttttgtcagtttttcctcttgttgtgtcccccacatccaaatagtgcctggtacttagtaggtgcttaaaatatttgttgagtaattgtaggagtgaatgaaagaataaatgaaagatcaaaacaatgataagaacatgaaagtgggttccaccaggaaaagtggggtatcctgggcagagaaagagagagtaaggattttacaagagacaatatagatattgccttgcctttagctattttctgaatctcagctaggagtatatattttaaaacaaaactcactgagaatagcatgatgcagtcacttaccagtaagtgtcacagtggcaataaacaactggccttaaaatacaaccattttcccagataaatatagcatatcattgagctaatcaaaaatggattcttttctcttatattgacaaatctctactctgaacttaacaccagagttagccaaattttaataaagatctttaacagcaaagctagttaacagttaatattaaagcaaatccaaactccggaacgctctcattttctattgtttttcgtcacttccaaatcctcccaaacacaatagactcaagacaagtcttggctttgtaccagtggggaaaagtctctcttcctcttctatttagatcgtgccacaagagcctaaaggctctcctggtggtacacaaagagttctttacattagaaatgcataaaatgctttctagctttgttctatagtccttgacccggtaactggacccaaaaagtaaacttctgggctctgatggtaaagaacaagtgcagtttatcagaaaaacgtacttagaaaaccacattagaaagatgcatcagcaaaatattctgcacacctacacccaaactttcagctcctctctgtctaattcaatggctacctgaactgaattcattctcccaatcccatccctttcattatagtttttatttgtttgaatctgtgtgttaaaacacagaagagaaatttgcaggttgaaatgctgtcttcaaaatcataagctggaaaataggagttctagagggtcaagattttaacatgaaagagatgtacagaataacttcactgaaaaataaagggaaaataaatgagaaccactgggagagaagagaaaaatacatgataaggaaaaagaaaacaatacagaataaaagaaagagaaggaaaaaggtataggagggagaaagagaaggggaaggtataggagggagaaagagaaggggaaggtataggagggagaaagagaagggggaaggtataggagggagaaagagaagggggaaggtataggagggagaaagagaaggggaaggtataggagggagaaagagaagggggaaggtataggagggagaaagagaaggggaaggtataggagggagaaagagaaggggaaggtataggagggagaaagagaaggggaaggtataggagggagaaagagaaggggaaggtataggagggagaaagagaagggggaaggtataggagggagaaagagaaggggcaggtataggagggagaaagagaaggggaagttataggagggagaaagagaagggggaaggtataggagggagaaagagaagggggaaggtataggagggagaaagagaaggggaaggtataggagggagaaagagaaggggcaggtataggagggagaaagagaagggggaaggtataggagggagaaagagaaggggaaggtataggagggagaaagagaagggggaaggtataggagggagaaagagaaggggaaggtataggagggagaaagagaaggggaaggtataggagggagaaagagaaggggcaggtataggagggagaaagagaagggggaaggtataggagggaggcccacaccgggaaggtataggagaaagaaagagaagggatccatacacaatgtctcgcctcccnacagacncagannnttgtatcaccaccgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctatttgtacatcatcaaaatgtagggaattaatatattaaatttattcagattaaatcttcaaatatataagtaactttttcattcatggtatggcctgattcaaaaacataataatcttcattaagaaatcacttatttctagtatatttccatatttatcatgtttatggttttgaatttttttatttcatttctctccaaactaatagattagatgcaattctaatcaatgtctcaagggtgtgcgtgaatgcgtgcttgtgtgctttagtgggatgtgacaaactacccctaaactttatgttggaagggcctagaataagtaagatgatctttaaaaaaatcaaagtgggaggccgaggcaggcggaatacgaggtcaggagataagagaccatcctggctaacaaggtgaaaccctgtctctactaaaaatagaaaacaattagccaggcgtggtggtgggtgcctgtagtcccagctactcaggaggctgacacaggagaatggcgtgaacctgggaggcggaacgtgcagtgaggcgacattgtgccactgcactccagcctgggcgacagagggagactccacctgaaaaaaaaaaaaaatacaagtggaaagagttaatattccaggtatcaagacttattataatgcaacagtaattaagcattctagtatttgtgcaaggacatagaaacacacagtggaacagagtcacctaacagactctcatgtatatagacagttaatgactgacaaaagtggcactgaaaagcagcttaatacagccttttaaataaatgagatggggtcaactgggtaatcatatgaaaaagataatgaaagttgatatccatctcacaaatacaaaactttctaggatgattataaatgttaatgtgaaagataaaataacaaaccttctggaagataatatgggaggtatcttgttgcttatggagtaggcaaaaacttcttaaacaggacaataccctggctatagaggaaaagactgataatttggagtatattgaaagtaaatgggctgggtgttgtggctcacacatgtaatcccagcgctttagggggctgagatggaaggatctcctgagctcaggaggttgaggctgcagtgaactgtgattgtactctagcactcaagcctgggtgacagagcaagaccttgtttcaaaaaaaaaaaaacaaaaaattgtcaaaagaaattatgaagaaagcaaaagcagctgggcatggtggctcatgcctgtaatcccagcactttgggaggccgaggcaggtggatcacagggtcaggagttcgagaccagcctgaccaatatggtgaaaccccatctctactaaaaatagaaaaattagccgggcgtggtggcagttgcctgtagtcccagctactcaggaggctgaggcaggagaatagcttgaacctgggaggcagaagttgctgtgaaccgagatcgcaccactgcactctagcctgggtgacagagcaagactccgtctcaaaaaaagaaagcaaaagctagttatgaaacgattcacattttcttttttgtatgttgattttggtaaattgcatttttttccttaaaaattatgttttatctaatatttttaaattttggtgtaaagttattctaatatcttactactttaaagcctgtaggatcaatgttatgtttccttttttatttctgacattagttatttgtatcaatacttgattatacttgccagaggtttttaatattattagcttttccagaagacttctttggaggctatttattaacacttaaatctgtttccttctttctcccttctttgggctaaattgctggatctttttctctcctaaattctttagatggatatttaacatactgatttttgtaaccttctatctttttaatatatgcatttaaacctacagatttctctctaaacatgactaactgtatctctaaagttatggcaaatatttatattatccttcatttaaaaaatattttaaaatgctgactctgttgtctttttcaataatgtgtgatttagaattgtattgcttgatttccaaacatggagactttgttttatcttcttttattattgatttctaatttaattcattatattaagagaaaacatactcttcatttcagtatgttgccatttattgagatttaccgaagatgtgatcagctttcatcagtgtgctgtgtatgcttgaaaagaatgttttaggttaaatgttttgcttcagtccattaggtaaattgctgattatgttgttcagatcttccatatccttactttttcttttcattgtagcagctattgagaaagttattgctactatgtttgtggacatgtgtttttccttagagctctgccaatttttgctatgtgtgctttgaagccttgttatttgaagcacgcatatttagaattatttattttccttatgaattacatttttatcaatagaagactctcactttgtttcttatgatgctttttgctgtaaatcattatctgttattaatatagctatacttgccttctttagtttaaaatttacatgttatatctttttactttctagtaatcttaatcatttttataccttttcatttgtatcttcaataaagagcgtaggtttggagctcattttttatcttgacagtcttttgttttttactgaagtataactaagtccatttacatttaatacaattgtttaaatgcttggatttaaagtactattttattgtatattttctatttgtcttatctgttctatagtccttttccttttgtgtcttttggatttatcaattactctttactatttaattgttctctctggtattttaaaaactgtaattttaccaactatcttttattattattatttttatttttattattaagacaaggtctccctctgtcacccatggtggagtgcggtggcataatcatagctcactgcagccttgaactcctgggcttaagccatcctcccacctcagcctctcaagtagctaggactacactacaggggtgcgtcaccaccccaagataatttttatgtttatttttaaatttctaaatttttttgtaaaggcaagattttactatcttgtccaggctggtcttgaactcctgggctccagcagtctttccgccttggcctcccaaagtgctgagattacagacctgagccactacacctaagcttatcttttaaaactgcatacatttttgccttttttgttttttggtactttcacctatttcctgacaaattaagcaaccttaagacctgtttccagacaaattaagcaaccttgagattccctaacttaataaatttcacttccaagttacacattgttattttattttaattctttctatattatctccacaggacattaatactgtttatatgtttagtattcatttaatcttttttctttgttctttctttctgcatcttcaggaatctgtttgggattattttctttcagcctaaagagcatctttttgtatttcctttagtgcaagcctgctaggtgacaaattctttagtgtttttggtcttaaaataggtttctttcattttcttcttgaaaaatatttttacaaggtgtagaattctagtaggcagtttaaaaaagatcattacactatcttctagctttcaatgacactaacttcttaacaaaaataatctgtttattcattcattgaaggtcatctatttgtggctgctttaaagatgttatttttagcttttttatagcagttttactgtattcacccacttatagcatttatagggctttgaaatcagtagtagtttgatactttctttcattttatgttttcaggaatgatctttctatagataatgcttctaagttattctctttatgatatctttttttctgggactccaattatacataagttagagttcttcactgtatcttttatcctcttattttttctgcaatatttcccatccttttgactccatttcttcattctggattgtttctttttatccttctttgaatgcactaattttttattcagctgcttctaatctaccattaagtagatctgttgaggttttaatttggctatttgtcagttctagaattgctcagtttttaaaattatttctagttatctgttggcattttttctttctgtttaaaaatttaaatcagggtctcatgttgtttcccaggctgaagtgctgtggtgcgatcatagctcactgcagcctcaacctcctgggctcaagtgatcctctagcctcagcctcccaagtagctgggactacaggtatggacaccacacctggctagattttaaaattttttgtagggacggggtctctctgtgttatctaggctggtctcaaactgctggactcaagcaatcctcccaactcgacctcccaaaagtgttggcatgactggcatgagccaccactctttgctgaagtctattttgtcttttatcttcttggagagatttaacatagctattttaaaatgtgagtctgataacaatctaggatccctgtgtgtctttttctgttgtctgttttcctattgatttttgtttattttttatttatgtctccatttttctattttttttttttattttatgctgtgcaaatgcttagtttgatctcatcttttcccagataggatttatgtttgcttttgccggggacctggggcactaatattccaagatcaccttgaggtatgttcaaagatttagatgctttgaagttgaattttagccctgaaaatgttcgacctgccttcttctagttaatctttgttactagtgtgatggaattttagattctaaccccaagtgagaggtgttggtagggccagtctctcttagaggtcagggtcctccagtccctttccccggataatgtgtccacaaactctcacaggaatcctgtagggtgttgaggggtgggggcaaaaggatcccccatgagccaacctccgctcactaggtctgtgttctcccttgaatcctggcttgctaattttttcttaatttaatattccctttcctcttcccttcaagatatatataaactcacatttatgtgttccatgttttttatatatatgtatataaatataaaatatatatatataaataaacgttacctcattatataattatacgttatgtagccattagctcatgtattattattattaatttttttttggagacagagtttcgctcttgttgcccaggctggagtgcaatggcacgatcttggctcactgcaaactctgcctcccgggttcaagcgattctcctgcttgaacctcccgagtaagtgagattgcaggtgccagccaccaagcccagctaatttttgtatttatagtagagatggggtttcaccacattggccaggctggtcttcaacttctgacctcaggcgatccacctgcctcagcctctcaaagtgctggcattacagatgtgagccactgcacccagcctcgtgtattatatttacaatataaatttatagccaaatactttcacttggcaacataccataaacagattttacatggcattcaatgagatgtgattttaatagctcttgattgtcccactatatgtacaaattaaattttttattcttactgctatttttggaaactctactttttttcttttgtaaaaatgcaagtaacattggaataaatatattctagtacataaatatttttgcatatcactgatatattctccccttagagaagacttctatgagtggaattggtggttcaaaacagatgggttgactgggcgcagcggctcacgcctgtaatcccagaactttgggaggccgaggcaggcggataacctgaggtcaggagttcaagaccagcctggccaacatgatgaaacccaatctctaccaaaaatacaaaaattagctgggtatggtggtgcatgcctgtaatcccagctacttgggaggctgaggcaggagaatcaaaccaacttgggaggcagaagttgcagtgagccaagattgtgccattgcacttgagcctgggtggcagagcaggactccatctcaaacaacaacaataacaaaaaacaaaaaaaaccaaaagggataggtttttaaattattttgatattaaatttttctgtagaaaatttgtgccaatttgtatctcactggtagtaatgttagtgcttcttttaagcacagctaagaaaacactcctttgtcatttttattttattttgggtgggggggacagaattttgctctgacacccaggctgaagtacagtgatcacagctcactgcagcctcaaactcctgggctcaagtgaatttcccactttgtcctcccaaagtgcttggattataggtgggagccatagtgcctggcctatttgtaaacatgtcagagatctcagttcatcctggggtttaatcttttgatgttatcttattattcttttacatttgtcattttacatttgtcattttacatttccatttttaaataagcccttttatttaaaagccaaccatagatgctttgggcaatcacatttatgtgttccatgtagagctgttttaatggctatgttatagtgctgtgtgaagattgtggcagaaatagaaattataagtgtgaagatcatgcaattatttaaaaaatctgtattatttataaaaccatgtgcactatgattacagctacataaaacattcaaagaaaatttagaaaataggctgggcacggtggctcacacctgtaatcccagcactttgggaggccgaggcgagtggatcgcttgaggtcaggagttcgagaccagcctggacaacatggtgaaaccctgtctctactataaataattggccaggcatggtggcacactcctgtaatcccagctcctgaggaagctgaggcagaagaatcctttgaatgagccaagattgcgccaccgcactctagcctgggtgacagagtgagactctgtctcaaaagaaaataaaatttagaaaatatacacagtggatgctgtggtattatgatactgattagttattgtttttcttatcttcccctctcaaaacaattctatagggttgtgctattattttttaataattaaatgattaacgcaaacccagatattgtgagttcaattcaggctactgtcagtagatctaccttttatccagccagtcagtcatcttataaatggttcaaatgagttaaaataaatggacatctcagctgggcacagtggctcacgcctggaataccaacattttgggaggctgaggtggaggatcacttgagctcaagagtttgagaccagcctgggcaacatagcaagactccatctctccaaaaaacaaaaacaaaaaagctaggcatcatggtactcacctgtagtcctggccacttgggaggctgaggtgggaggatggcttgaactcaggaattgaaggctacagtgacctgtgatcacactactgcacttcagcctgggccacagaatgagaccccatctctttaggtaattaaataaatacggcctttttgttatttccccaatagtaataatgtgtttattgctacctagtccaaaaagtatttacaatgattaggaaaaaaagatatagtaagaaaataaaaaggtgctattttagtgaaaagattaaagaaaagaaaaaacaatactgaagtggaaccaggaatgagactaatcaaaaacacatattccagaatctccaaacatgtggcaggtgggccatatgtgaatttcactgtttttttaaataacaaaaatgaaaagcacatctgatcaacacaatttatattgtccctgagattaaaaaaaagaaaacacaaatggccaggtgtggtggctcatgcctgtaatcccaacactttgagagggccaggcaggtggatcatgaggtcaagagatcgagatcatcctagccgatatggtgaaaccccgtctttactaaaaatacaaaaattagctgggcgtggtggcacatgcctgtagtcccagctaatctggaggctgaggcaggagaatcgcttgaacccgggatgcagagactccatctccgtctcaaacaaacacaaactcaaaataccatccaactatttagttctttagacagagctgtgaaattataagatcaggaataaatgtgtcttcagagccctcctacacgagatattctgtaatgggatgggcattttcagtagtcttctgtggacttggcagtcttgtctcagatatcacacccaagcccaattcagttgaggcaattctttttgaggtcagtaggacaaggtgtagagccctctgctggcctgagatagatttatctacataatctagggatagattatatttgatttgatttgatattgttttgttttgtttttgagacggtttcactgtgtcgcccaggctggagtacagtggtgcgatcacagctcactgcagcctcgacctccccccgggctcatgcattccttccgcctcagcctcaagtagctgggaccacaggagtgtgccaccacacttggctaatttttttatgtaaaatttttttttctgtagagatagtcttgctatctcgtcctggctggtctccatctcctgggctcaagtaatactcctgcctcggccttctgaagtgctggaattataggcgtgagccaccagcacacctgaccaggacagattttagagtggttataaagaaatcagtgagctgcatacttcgaggcaatttttatttttaatatttttttccccttcatgcaaactgtggcatcaggtgggagatagtactttatggaatatctgggatatagttaccatattttgctactttaatatgggtctgctggccttgatagacaattgagctggggcagtgctgatagtttcttattaagagaggaacttaaactcagattaattcccacacagatggacattctgtctctactcacagataagccaatcatggaatgagaatagcaacagttcctctcagacagtaataatctaggttctgcattaatatacagtccatccctggcgccgaccagaacccgtggacatggtgaaccaggttatcaagtgcaaggctgcagttgcctgggaggctggaaagcctctctccgtagaggagatagaggtggcacccctaaaggctcgtgaagtttgaatcaagatcattgccactgcagtttgccataccaatgcctataccctgagcagagctgatcctgagggttgttttccagtgatcttgggacatgaaggtgctggaattgtgggaagtgttggtgagggagttgctaagctgaaggcgggtgataactgtcatcccattttacatcccacagtgtggagaatgcaaattttgtctaaatcctaaaactaaccttgccagaatataagagtcactcaagggaaaggattagtgccagatggtaccagcagatttacttgcaaaggaaagacaattttacattacatgggaaccagcacattttctgaatgcacagttgtggctgatatctctgttgctaaaatagattctttagcacctttggataaagtctgccttctaggttgtggcatttcagctggttatggtgctgctgtgaacactgtcaaggtggggcctggctctgtttgggccgtctttggcctgggaggagttggattgacagttatcgtgggcggtaaagtggctggtgcatcccggatcattggtgtggacatccatcaagataaatttccaagggctaaagagtttggagccactgaatgtatgaaccgtcaggattttagtcaacccatccaggaagtgctcattgagcggactgatggaggagtggactactcctttgaatgtattaggaatgtcaaggtcgtgagagcagcacttgaggcatgtcagcagggctggggcgtcagtgtggtggttggagtagctgcttcaggtcaagaaattgccactcatccattccagctggtaacaggtcgcacatggaaaggcactgcctttggagggtgaaagagtgtagaaagtgtcccaaagttggtatctgaatatgtgtctaaaaagataaaagttgatgaatttgtgactcacaatctgtcttttgatgaaattaacaaagcctttgaactgttgcattctggaaaaagcattcgaactgttgtgaagatttaattcaaaagagaaaaccagcgtccatcctgtcgtgatgtgatgggagcagcctaacaggcagagagaagcgcctcctagaccttcagcagctactccagagaatggtgtgatgtgcgtcattcatgaatctctgtaatcaaggcaaggataattcagtcatggactggactctcctccacataaataattgctagctcattaaggaatattttaacataataaaagtaatttctacaaaaaaaatacagactattggacaatgaaattttcttgcatatggaagaaccagaaaaaatgttgatctgaaatattttaaggtgggaaccaaaccctcatcttacctgtaaaaatctcagcgaagcactcttagaatgcctacctttgagcattgttattttctggtggacacactatgataaattatttgtggattatagctctgagttattttaggtgttgttatttataacctagtgaaaagatggggaaatagctgctaaaagtaacttttctctttcttaagctagcaggcctgtagcctactttacgccacttttaggttgtgtttttaaagtttctcatatgcctatggtagaaagtttgttttctttaataggaagatacaatgtcattccgcaaaagccaaaacagatctgaagaatttaaggatgtggagtggagtcatgaaaatgcatccctgctttaacccaaaaataaaactgtgatttgctctgttaaacaattcttatccatcctgaccttgtccttaatactcacagtatgttgattatttatacttgacattcctgagaaaacaagggaatgtaggatttggtaactggtagctagaaactcatataccctattcgggaaaataacacatttgtgaaatattacacatacacatacacacacacacacacagaaaaagacatttatggatggttgaggaaatatcacagggcaattgttaattgaatgcaaaactggttgacaaacagcacaaatgccaagaaaactcagtttgctcaactacttatagtcatgcaaagagcacagaaagatttagtgctggttaagtagagatgatgtgtaggtgagataatgattttcaattcctctgaaaaggatttttttaaagaaataaagtgtgactattgtaatcaaacatagatttttaaaattcaaaacaaatagtattcatggcatattacatttgaatatcaccatgtaattttcatagttcttttcacatatcatttactcttccctcatgcaagctcccccaaaaagacaagggacacatttttcctgatttactgatgagaatattgaggctccaaaaggtgatgaggtttgcttaggggggagcaatgaatatatcttaggaattttatctattccatgttttttctttaaatagttaattaccaataggtttagacataccagataacctgataccttcctttttccatatgatacccacaaagtttccttttggttccttggtaaacaaatgaagaaaatgagattagcagctacagcctctgcctatattgaccagccatctcttttatttatttttacttgataaatgtgttaattcagtaggcagccagtggcccttttttttgttttttgttttttgtttttttttagatggagtcttgctctgtcgtccaggctggagcacagtggcctgatcttggctcactgcaacctctgcctcccaggttcaaacaattctcccgcctcagctttccgagtagctgggactacaggcgcctgccaccacgctcagctaatttttgtatttttagtagaaaaggggtttcaacatgttggccaggctggtctcgaactcctgatctcaggtgatctgcccggcttggcctcccaaagtgctgggattacaggtgtgagccaccacgcccggcctcaagtggcttttatgtagtcatcatagcactgtccagagctaggggagatttacaagacctgtgtccacccttctcatcctgaagtcgtttgcagtcaatttgaggaaacatgatatacactcaggactaggggacaagccactgttttggatagtacaccattaagtgttgaaatgtggggtgacaggtgtgctgagttagctgtcccattcaggagtggcctttcagcagaggtgggtctgattcaggtgaaatgggacttcaactagattttttaatgattgattagactccttgagtgggtagagctggagaaggcattctagtggcatgaagcttggtttaggaaacaagaaatgaatctgggaaggaagatggtgggaatataggaacgttgaaatgatatttaagttgaagagttgggatttgattctgcagacacggaggaaccatggaagaaatctgttttgcttattttctgctagtggagctacgtgatgaatgggaggctttgagattactgttacagcccagtaatacattacagttctgaggtgtggttcaaagacagggggactgaccagatacttttgtttgtttatatgaggttttatagagtgcttttagcatgctttgtattttggtgaatgatgaaactcagtgagatagtttatccccttggttgttggtgctacctcttttatatggaaaaaactatgtctgaggctgttagtaggatgatggctctctaggcaaaagtctttttgtggttgatcatgatgccaggtctggagcctctcagtcactccagccctgcttctctccaccaactcccagaggagctggtaggccaagcatttttctaagcatgctatagatagcatattttacacatgaggaaccgaagctcaggcttagaagcatgctgttgtggccaatttggttctgctactctggaaaatattaggagggcaatggctgcaaagatttaagataggctgttttctggggctgtgtagaagaactgtagctaactgtagctaaaataggtatcattaccactaactggagggcggtgaaggatagagtggagagggcattacatttcagaaacttcttttctttctgtttttttgagacaaggtcttgctctgttgtccaggctagagtgcagaagcatagtcatcattcattacagcctcaaactcccaggctcaagtgatcctttcacttcagcctcctgagtagctaggaataaaagtgtgtgccaccatccctggctaattttttaaaaatttttaaatattctgtagaggtggggtctcactgtgttgcccaggctggtcttgagctcctgggctcaagcaatttacctgactttgcctcccaaagtgctgggattataggcgggagccacatgcctggctacagttcagttgttttcttaagccatcacagtatccatgttacctcttaagaactgacttgtcaattttatcatgtgctgctcttgaataagtgcatcttagctatccccaacagccttgcatttactgtttttaaaaaaattattaaaaaaattttaactgttgcaaaatacacagaacattaatttaccaccttaaccattttaagtgtccaattcagtgttgttatgtatattcacattgttgtgcaatcaacctccagaattttttcttcttgcaaaactgaaagtctgtacccgttacattgcaacaacttcctatcccccatctctccaatccgggcaaccaccatttgactttctgacaatatgaatttgacaatgttatgtacctcatatgggtagaatcatacggtattcgtctgtttgtagcaggcttatttcatttagccaaatgtcctccaggttcatccatgtggtagcatgtatcagagtttccttctatttaaaggctaaataatttcccagtttgtatatatatcacattttgtttatctgttcattcatggatggtcacttgggttgcttccacctttcggctgttgtgaacatgctgttatgaacataggtgtacagatgtttctttgagatcctgtttttaattctgtggcatatatatccagaagtgtaattgctggatcatatagtaattctatttttaattttttgaggaactgccatatattttccatagcaacagtatcattttacggtatcactaatagtacacaatgctttcaatttctccaatccttgccagcacttatgttctatcttgttgttgttgttttggatagggccttgctcttttgctgagctggagtgtagtggcgtgatcatggctcactgcagccttgacttccggggctcaagtgatcctcccaagtagctaggactacaggtgtgtgccactatacctggctaatttttatatttttgtagagatgaggttttgccatgttgcccaggctggtgctttattttgttgttgttgttgtttgtttgttttttaaatagccattctcttgggtgtgaggtgatttcattgtggttttgatttgcgtttccataatgattagtgatattgagcatcttttcatgtgcttgttgactattttgtgtatcttctttgaaacaatgtctattcaagtcctttgcccattttaaaatcaggttatttgctttttgttgttgacttgttaggagttctttctatattcaggatgttaaatccttatcagggttgatttgcaaatacttcctcccattttgtaggttgccttttcactctgattgtgtcctttgatgcacggaagttttaaattatgatatagttcagttcattgatttttaaaaaaatttttgttccttgtgcttttgttgtcatattcaagaactcattgccaaaaacagtgttaccaagtttttcctgtatgttttcgtctaagagttttgtagttttaggtcttatgtttcagtctttgttccattttgaatttttgtatatggggtaagaatttactgggttttaatgaaagcaaatctcaaatgaagttttgcccaacatgtaaacatttattaaacatacaacattagtttgcacaagagcctatccaagctgacttgagagtcagagcgtggccagcttgccactctggtaggtgcttgacctcaccttgtgtgaaaggtccatgtgatacaaacaccaaggaataggattctgtgaccgactgttggcaacaggcaaagagacatcctcctaaactagaggtgggaggtagcactctccggctgtcacaccttgacatcatcccttgcctgagttttatcctttttaagtgtggggatgttctctgatgttgtactcaatctacttacattgaattccttcaggttctccaatgatagctcattgtctaacattcattatgttaattaaagccactaatttcaccaatgaaaaactatttctgatattcagtatgctaaatagcgcagtttctggtgttggtcgagataggatagctggaagatacatgagtagctcaaatgcttagccaggtctgaaatgtggtcctttctgatccaacgctgaggctaaccccaggaatctgaattctcagttaggaccaatgactgtccctaaatgtaaagcttaaactaactggtctatacattgtatcttcctgttcatgctggagattattttatgaatttcttaatttgttaaatacagaaatagcaacatccatccgataactcatttactttgtagtctcgacccagtgattacagtggttttttgtttttcacttccaccatactgttcttctctttgctttacttgatgtggatcaaaggtattagggaaatacaaccagttaagaaaaatgtcacaaatatagggcatgcatatattgagcatatgcatcctaaaatggttgaacacattgttattctgaaatcctaatgagcagttttccaaggtgtttataggccatggagcaggcttccctgtgagatttgtagtttgtctcctggccctaggtatagtactagctgctttctcattctgtccagattctgttggtctgtagctggcgaggtctttcttggccagaagatccctattcaattctttttttttttttttttcttagcaggttcattcactgtttcatggtttctctcccagtttccaacctggaccccagataagtcggtttcctgggccttttgtggttgaaggcacgggagctattattgtcctgtggtgcccaagtacagcctgtcctttgacattcccacaagtatataaacaattttaaaagggatatcatattcagatgaattaaagatgctagttcagggagttctctgattgactgtctggactatctgattttataattccctgcagggctacatagcatctttccctccaaagaaagaagcgtaggttttactatagaaaatgaagtgaagcaaatgtggttataaagaataaatgactgcgtgaaaaaaatgttatcttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctcactgtggttttgatttgcatttctccaatgaccagtaatgatgagcttttttccctatgtctgttggctgcatacatgtcttcttttgagaagtgtctgttcatatcctttgcccactttttgatgggaaagggtattcttaagtagaaagtatagcctgtttgttatccatgcaaatgacctctaaggaatggaagaatccagcgtataagagggatcaaaccaaatatggtggctcacacctataatctcagcacttagggaggctaaggccaaaggattgcttgagaagtttgagactagcctgggcaatccctatcatttctgcaagggaagtatagtatgaatgctgggcagcagtgaagggcccacctgaggttagtggccatgattttaagggtggtttgcatttctctctgcaatgttcagctgttcaggacacaggcattagaataggtgaagaggtggttttaacaagggttgaggtcttgtcatttgcatatgatattttattatagaggaattaaagggtgtatatgaccctggaaggcatgaataatggtgcgtggttaagaatgtgtgttaggatgtaagggaagagtgtgagggacaatataatttgaggagaagcttcatgtgtcctggtgaaactgatgagaacactttgaccctaccttgggggttaagtagaggataacaatgcacttgagatgagtcaatcatctgaaataatcatgtgaacacttagttgcaccaaagcaaaaggaaaatgcacaattcatattgttagtggaagatgcagttatctattggggtgtgtgtcagtgttttccatccttttccaaatgacaccccatttgccaccatgaccctcatggtgcagactacacaggctgctcaattttccacaatcatgtaagtttgtaaaatatcctcaagattatatactgcttgaatctactatgttgcatgtagagtatttctagatgggtaagaatcagatcaagtggcaaggacgaagagagatgtattctatattgagcatattttctatttttcgaggctctgcctggaaaggggacagaatgctcagtgttgcctcattttctcatcaaacttgacactagttcactggggaaattgcatttccaagttgttttaaatcaggaagttgttttaaaatcaggctgtgtgtttgtgtttgcactgttgccacttaatccttgatcctgttaaatgtttcttccatacagaggaacataaacaatagcggctttcttgatttcctaacattcatgtaaagataagttggaaatattatatttaataaacgtaaaatactgtaattaaatgtaaacagaaaattcaattaccattttggtggattgttggataataacattgaatatttgtcacagtagccatttggaagagaatacagattgagttattgatgggatccaagttgacattaattatttcattagaaaaatatatttcttctaattcttagaattttttattttaaaatctggtatgatcaatccgtttaagaccagcaggtctaaactctcccggactctaggactgccatgtttgaatacagttatattgaacctttctgccttttctttttttttttttcaaataaatggagggagaaggatgtgttttatgaagattatgccttacccaactttaagtacctgcatcttcttctttttttttttttttttttttttttgagacagagttgcactctgtcgcccaggctggagtgcagtggcacaatcttggcttgctgcaacctctgcctcccaggttcaagtgattctcttgcctcagcctcccaagtaactgggattacaggcacctgccaccatgcccggctaatttttttgtatttttagtagagacgaggtttcaccatgttggccagcctggttttgaactcccgacctcaagtgatccgcccgtcttggcctcccaaagtgctaggattacaggtgtgagccacagcgcctggcccgtacctacatcttctaacattgccttatttaccagagtgaaggcttttgaaatatttagatgtctggtaaacaataaacttaaaaatattgagttcgttaaaaatggaactcaaatgtttatttaaattctaagaggctttactgttttttgaaatatttgaaatgtttagaatagttaccaacatgcatactttacacttttgagatgaattatttctaattcaaatgtacttatcagtggtccaatgaaccccgattcctattacactgtgcatatattaatgcttcttgctaagtgcccacttttaaaagtttatttcataactatataaacaggcatttggggctcctggcctcactgctgcagagtgtctggaatgagcatttgtagggtcagttgtcattatcttggtcacgatctttggagtgctgtattcccatcacctgctgcacatagcacaaggtctgggtgtcacaaagacatgacgtgactcttctagcatctgtgatactcttgagtagtaatttcagaatacttagtgaaaggctcgaagtgttgaaatacgatgaagtctggggagttaacttttagcctcgactttaactcatttattctgtgccaaagaggaatttgggggagaggttgagttttcctttgtggtagcctggcaggtgaggacaaatccacttactctcctctttcctcagctagaagagacgaggaatggaaggagatttgatattccttaggaattttattaataggaaattgcttcttctttaacaatgaggatcataacttcagctgagattaacttttaagggctttattaaactgttttagccactagcaacgatttcccttacacagatacatttagattgactttgcacatcttgaaaataagaaacaaatgaaaataccagagtatctttatatgtataagttgtgaaatttgaagtctgcattacttcttattggttcagttttcatgggattatttatgtataggagcagccaaggaaaagaccatagcagatttttgtaatgatggaatgagattcctggcctgtcaagccagtaggtgtgggactatggagaaagttcctaaattttagtccagttatttgaatggaatcttgaagagacatttactattcatagagcaacctgagctcgaactgtaattttgttctagttgggcagtgggggaagagaagagacttgaaactagtgttatatgatggacacgtcagtgctgaaaattcacggtgcctgaggtaggttaaaccttcctccaaccaatggtttccatgcaagctttttaacacagtgatgcctatcctatatttttactatttgcacatattacgagcgattgaaggatgttaggaagctctgacaataaaaatggttttctttaatgaaaaaaaaattctgataccgtgagaagaggacagatacatcttgctcaattaaatcaaaatgtgtgggacacctcatttctgcagttgcagaaatgctgaaagtcactggatacttaaagcaatgaaggaggaagaggttggggttgggaaaagcaaaactgataatctcctttgtgaagacatcactgtaagaagcaggtggtacttactgaactctggagagaatggaaagtgtgaggacagcacagtaaatagaaggaaaattcccttagcagtccactttccaggcaccagtcacctctggaactttaattagaactcaaaaatgtgagtggaggaattagaacagaggcctttctatcttatatttgaaaagtatttattttacttagaagcaactttttattaattaaaagcacatcagataaatcctaatatttgaaacacctccccatatttaactaattcctcattcttctagacaaggaaacattaaagtaatttttacacatcaacatatacttcagaagagaaaaaagaaaccttcattaactgaacaaataacaaaatatgactcaacatacacaaaaaccagtgtatcatatttttgcatatattaactacatgttgatgtaaataggaacaaaattattccaaagggagggtgataaatatgtgggttttagatttttcatgtttttaaacatattggaaatgtaatgaaatacaagttaattaaggtgatctctgtagcacagtggaaggtattaccctaacatgtctaattttggtatgtgagctctgatcttatcagtcatatgttacaaattcacaactgagttttcttttttttatgaatttttaatttttagtgaccaaacttcttagttcttccccctacctcagcatgtctgccaatatataaatgtttacatatactaaagaagttgaatatttaaatgaagttttaggtgaactattttaaaaaggaaaattttttcccttaaaattgtgattataatcattgtgtttataattcaagcttttctgttttaggtttctttcagtgagacatgtttgtgtagcactacactgaaaaaagatgattcttattggtatgaaagttattttggcataagacattttagatttctgatagattctggacttatggagcaatatatatgcttacattcaaaataaagtttatcactgtgccccttttagagcatgtgttagaaaacagtggcaaaatgtgatcccagagttagggttctatttccaggggaactcactaagccctcttttacaagagcataaatgtttacctaagcaagaaggtgaaatctgttatgcccaataaaggacattttgcagtgctggtttacggaaaacattcaacagacagtggatgttttacataaagtacagtgcgttgtttgaaactctttgctcatctatacactatgcttaaaataaacactatctgggaatatacccgtcttttttctacctcccttattccacaccctagattttgtcacctgccaaagtcaaataaatcttatctaaacagttgagataagcgccgagtcgtacccagtgtggtcaaatcaacaaacctctccaaaaacagaaatgaaatacaatatcgctagattaaacagtctacaatggtaatcatgtttgcatttaaagattttattccaaaaggatcacagtcttggtatccatttttttccctcctgcctagacaagacggcgacatactgactcaaacaaaatgcatttgttctctaacaatttaacttgtacagctcttataaacaaggcccataggtgtatgaagcctaccgacttaatcagtaacttatgtcgttaaccttgtataagcaagtatgcttgagatgtaccagtgttggattgtgtatgggatttagttttttcttgcaggaaagcctttctttatgtagcctgagtgcaaagtctatggcattctgggattttattgtctgtatcttacagtaaagctccatcatgctaaactgttcaaaaagagaagatagtacaaattaaagtgatttctaaattataggatattataaaaaatgcagttttcttccttcctaggacatatggtattttaactgtttgataacaaggtgtaaactctactagggcaggaaccgtgtctgtctggtttatgctgtatcctcagggcggacaagagtacctagctcagaattagaattctgaatatttgttcaatgaatcaatgatgttttccagaagatattctgtcttctctgtggatgaataaaaatattttgtaattattacattaatttctgatatttaaaagcatgtctctcgagagtacatttttgtcttacagtttttaataaatggtctcctttcaatgtcgtaccatttaattagcaaaatttaaaatacgcaataaaggaaggacaaatgacacaatatcactcattttttggtaatatctctaaggtactgtatttgtgcatttcttactgtagagcaatttatacactgactgctggtttgaaaataacccatttttccattattctttaagcagatcttcatttactttggttcatgaaattgggaattagcccagttgtatatagatgcgtgatttgaatatactgtccttttatttttaaggtccaaacaataatacgaaacttatagtcttattttctaatttatattttgctttatgagagcaaatatatctacaaaggtatatgtgctatttagacattgaccgaaaactgaagaacctttcattttagagtgcctgctataacaaggactgttttccacacaatagaggagttacctctaatctttataatatccatttaaggggataataacattaccactgatatgtctatttcccatgtgaataaaccaaagctcatagagcttaccaagtctctccaagagatgcacagctggttagtcacagagccctgtttttagttcaggtctatctaaatccaaaacacaaggtctaagtctcatgtaattctggagaaaaccacaacttttgagttattatattggtgagttaagacttctcagtaaacatgttactgtttaaactagagcttttaatcagaagagagtaataaaatttagaagtcttacaaatacaaggaatagaacgccactatttaaatttgggatcaaaagaagaatgctgttggtatatgtgccatttaactggcctttatcatggtctttagataatccagaattacttgaaatattactcacgagcgtgtgtgcagggtttcaggtgtatgttaagaatgcctggcagcacgtcttctggcaagatgcccacgtttgtgtgcatactcaaaggccccaggtttccaggtgcccaagcatcttagtggtgagatgcagaaagaatgcaagtagtatttcctttcatcttatcatcgagtgtaaaatgttccgatgaatatggtagcaagtgtcttccttcctttgaaatatgagttttcatcagctgttggatatgctcacgtaactaagccaagcaaaatccaacacaaatttaaaagttgcttggctacctctttagaaaatttttgttatgtaatatacattttggaagtttttaatgtaactgcccaggtgtgcagattttcagctggagattgatgcatcagcattctattccttagaaaacaatgctgggtgtgactcttgactcctttcacatccagttgcacagtgacatgcacctcccgaaggtttctatgctttattgagtttcaaataaagattgagggatgatagttctaggcatttctgccaagctgtgtttttcttttttttcttcttccttcttacatcttctcagcagattaattagaacagtaattgaaataaataaaaattaaggcagccctgaaaataaaaatgagccttttgtatccaccacagattatagatgcagaggaggaaaaggaggcttcttcccctagtaatgtctgtctgttgctcttttctttgcagttcctctcctcattactatttagaggctcatgcttgtattaccatttgcagcagggatttttgcctatgccagtatttatttggctttcactttctcaggcatttcggcctttgtgcatttctggagccaataaggcaaatgtgtgagtggtggcatcctttcttaaggttacctattatgacttttccctggaggttcagcaatgacttttgtttttcacattacacacttcttcagttgtctgctatatgcaggacactctggcatgatacaataatgtctaagacaagatccctgctattaggctcttaaaaatcttcttggagcgaccaaccacatacttttatacaatagagggagtagctggagtgtatcagatggcttttattaatgttaaagggtggaatccaaaggtagggaaagtcatgagccttggtatttatggagtgttttggagattacttagactggagcttgaaattgctattttgttagaaacagcagttcgtccacatttagagccatactttccagtttccctttccttccaggtgaccataggaaactgatccggtcagtgagatgtaagtagaagtctcctaaaaatttatgatccatcggcattgtgcatttcttcttgttgctttcctctttcctccttcgtctggcctggaatgtgaaagtgatggctggtacagtagcagcaattgttccaagtgaggaaaaggaaaaggcatgtgaagaccacttaaaaattttgggccctaacatattttatcccctgaatcaatggcaacctaccttagctttgttcaagtctctatctactcagatattcttttacttactgctgaaaacaattttttaactgattcaaatgtatttagcactttggccatattgtcatgtgggtaattgtcaggatttcattcatatttcaaataatcttcatcactatattctaactagatgcccatatcactccctaatatgagccaatcttttaagctgggggggaggattgagttagaaacaaacagtcaggaatgactgacattttccccctcatctagaccctgtgttttcattttacttcctaataaactcaaagatattggaattcagtttttatttctctttaattccactagaatacagatgatagcttctataaaagcactttcccttccccagctgtccactcttggaattctttgtccatctacacaacttttcctcaactttatattgttttgtcttttaacacctaaagacaaacacaaacttagctaggtaagaagagggaaatatttctactattagatgaaggattataaaagaaaggcagagaaactttctaagttgtaatctgtcttctagcttcagagtccctcaaatttttaaggaccttttctgggtccttaaaaacgtaaaaactaataggcaatgtagtgagtaagttctgtgaacagaaggatcaccagggacacagcttctggctgggctagagtgtgaaccaagtgctttatatctggcaggcatatggtcagagggagtacgtgattctatggtttttgttaatcttaactcgctgggatctactctcaggaactggtcaaccttttctttcctgttatttggatgttggccttcctaaatttgtctaaatttcttggctttttcctcttcgtttcctttttgatctacttttgggaaattctcacatgtatttcccaaatcttctattgggtttttctgttaccctgttttttgtttgaagaaacattttgttcaattaggagggtgttgcaagaattgcaggcaataatcgggaatgtctgagaatggggtggtaggggtgggtgtggtgaggtgaagtcatattctggatgtgtttggaatgtgaagccaacacgattcatgatgtgttggatgtggaaggtgaggggaagggaggaattaaggacagttcctctgaacaccaagcagatggaggctccgtcatgtaagacagaagaacatgtatagagtgtgctgtctgttagacacccaagtggtgatgctgagaaggctgttggttctataagtgaggagttcaggaaggaggtcggaagctggagatgaaagtgctggagtatttcaagcccaccgtgagactgggcgagaccaccaaggcggtgagtgtagatagaggtaagaaagggtggcagtagtgagccctaggacattccaaaaatgagaggttagggagagaaagagccagcaggggagactaaggaatggggaacattagaagtaggaggaagaggaagagagagtggtatcctggaagctaggataaatgtttagcacgtgtatcggaaggaatttataactgtgtcacatgctgccaagggttgagaaatgtgcatttggatttcacaagatctaaaacactggtgactcagtaatcagagcaaagacttgatgagtgggtttaggaagtgatgggcagagaagaaggtggggacaacaagtattgacgacactttgtaggagttggaagagttttactgctttatttttttttcctttttttcccccgctctggtttctaacatcccttgtagaggcttcccatagatgatttgatccttggctgtctgttcttgcttaagtctaaaaggtgatgagaagccctgagtcttcatgttgggctcttggactttgagttcagtgtagcattttcggtgtgggttttttgtcgggtattcacccatgtctttatcttttggtcttttttcctgggatggagagatttcctggagacttcccatttctggcttggggtgtaaaggcctgtctggcagtgttctggaccctaaattgggggaggagggctgaatggaggtatggaggcttttgtattcaacatgcagtactccacatggtccctgtgtattcagcatagcaacctatcctacaattgtgattgctgtgtctttgtccagagacttagatttaatttctcaagaaaatcaacctgatgaagttgggaaggactttttcagaatgagcatgtaactgactgactggcagcattcatccagtgctccttgtttctgatattccttgctttaccctcagttggacaagtgcctgttgccagttccttggccttcagggttggtgctcagttttctcagtgaaagttatggttttatcattttaaggtctacgaaactgagtagagctggcttccacattttgttgctgtcatctcatcttctttcttccagtccttatgggtttacttttttaaatatgtttttactatagctttagtgaaacttcaggagagaacaaaatcaaatgtgtgtacttcctgtgccatcttaatcagagatgcttcccagcagtacgtattaatacatatcacagtgcaaatggaatgaagcaatctgaatgttaggtgatgctattaagatttagaccatctatttagtagtcgttcactttcaaactctgtcactttgaggatcttttacacctgacaacgcagttctttctgctgtttgcacaagctctaccttgtgcttttattgtggtgtaattgttgtgtagttatttgtcgatgtgtctttctacttcactatactgtcagctcttctagatgggggagtgtgtataactccagattcccaaaatgttttgtaggacatttacaaaagagataacaaatactgtcagatacatttcaattacgcatccccaaaatgtaaaagtttctatttcactgaaatatacagtgaaacaaaaattaactgcttacatgtcctcgtcgtcctgattttaaagtcgaaatgacatgctccctgcagttcctcagagagatctcttacaaaatgtagtttcctggtagctagagtaagatgacaaacctgactcttaaatgagaggatgcttctgatctctgctacataagatgagtgtctgctcaaacaaaggtctttgcttctcagcagcttgcaaagctaacttcatcaaagcttttcctgtggcttctgtattttaaatggggtaaagatgctttcttcaagcccttatactctgccagtgaaaagagggcatgaaccgtaaatatgggctgatttgtactcattttgtgtttaagcttctgctgactctacttctctttccatgtactttaacccaaattagtgttaatggcttccctctggttacttgtttagtcttctatgttactatcagtacaactgaaactgtcatttttctttttgctgagaatgttttcaacctggttcatctgaaacctctcaatctaagatactgatttggagagagattaaaaaaaacattttccattataggaatgtgaaatgggccgtaataataatcacaacaaccaacattttctgagtgtttgctttgtgcgtggcactgcatttgacttttattatttaatctcctgcaagaactctgtagggcaggtcctcttcttattaggtccattctgcagatgaggaacttgagactctctgggttatgcagataataagttgtgtggtcactgtgccccactgccttccagtgccatcaggtggcaatgtgacatgacatattatctttgtgttctgtatttttgagctatgatgtcagctagagtgaagttagttatttatgaaaggacaatcagagtgatctcaaagaaaggtcagtttatccagtaaaattaaaggcagagttgattttcaggaattgctaacctcagcctgttttagcctttcagcattcgagaaagatgtttttccaagtcttttgtgtactgggttcttctagatagagaaaggatacaacagtgaatgaagcatgaccatgtaattatggagggaagggtctgtccatattgtctcacacgtctctgtatatctacagaacgtatctcatatgatctctatttatagaaatgatgaggtttagctgatttgataggttatgcctaaaccagaagccagccttcctgactagtgcactttttctttccactgaatcatgatgcttctccatgtcttttgtcaacctagagcctaataggagaataaaaacattcgaattaatgaaattaacacaaggtagagacccatagagcaacacaagttggaagctgaagtccaaggaacagagagccatgggttagcaatgtgttgtgatgggcagcccctcagatggtccctggagatccccaccttctgagactcacggccctgtgatattccctagcccttatatgtgggctacactcacgactcacactgaatagaatagaacagaacaggggtgatgggatgtcacttccaaaataaggttacaaagagaaggaggccctgtctcgtatgccctttctgtctcactctaagggaagccaactaccttgagagttgtctatgaagaagcccatctggcaagaaactgagtcagacttccagccaaccagtagtgagatgctgaggccctcagcctgaggaactgaatcctgccaacagcctgtgagtaaggctggaaacagatcttcctgcagagccttcagatgagaccgtagcccccgccaacactgtgattgcagtcttatgggagaccttgaggcaaaggcccctagctgagctgcacctggatttgtgacccacagaacgtgtgagataatccatgtgtgctctgtaagcccctaagctttggtgtaacttgccctgcagaaatgaataacacacttggtaataaacactgtgagattttctagtttggggcatctttaccaccagtttagcaggtcacacaggaaagattaaagtaggtgactgcagtggctgggataagtgttcatggcttatgaaaggaaggggccatcaagcacagccctgagagactgctccagtcctgtctcatccaacagatccctgattttaaaaaatatcaatgtaaggcatgtactaacactgttccatagcttaaaatgttgcattcaacaagagtaatcctatcatattgaattatacaaataggttgggcaccgtggctcatgcctgcaatctcagcactttggaagcctgaggtgggaggatcacttgaggccaggagttcaagaccggcctgggcaacatagcaagacctttttctctacaaaaaaattaaaaaaatagaaatattatctgggcatggtggcatgtacctgttgtccccgccacatggagaggctgagatgagaagattgcttgaacccaggagtctgaggctgcagtgagctatgatcataccactgcaccctagcatgggtgacagagtgagaccctgtcactctgtctctctctaaaaaaaagggattacacaaataaagtgaactacttaatctggctattcaaaatatgtgaaagatcaacaaatttgtctaataatgttttgttgaagtgctttgttttaaatgagttccttttaaggcacatttaaaataagtcctttcccttctgtgcagttctttgaaagtatagagacaggattcattttatcatgtaaacattcatacagatagattactttgctctctagcctgtgggtaattcaacatttgtagtaagttagaataagaagcatgtaccacatttctggtgctgacaaaggcagccacggaggagaacacgttaatagcaaagaccaagtgtccaaggatagacacctgcctgcgcacagtcaagagtgggactgtttcttatgttgagtctataatgcttggttgttttgaattgctttattcttttatttttttaatgtaatgcagtcatcgatcaaaatcttaaaatcaaaagaaaacagatttctttttctgtttggacattttgagacattttaaatatcttaggttaaattgacgattggacttccaaaacaggccatttcatttttcattttttaacagcatgctttaacaacaatgacattggatcttagcgagctcataccggtggagcaagttcgtaccggaggagttcatgatgagaatagctattgtgaaattaacttttaactattgaacgcaaatgtttagtcctgaacctagttttaatttagcacctagttttaatctaatttctgaaccaatgtgtttacaggtaacatacaccttaaaattaatcacacccattattccaagcctttctgaattatttttctactatataattacttattttccccacatgataagcatatgctataaacgaaagttatcccacttgataaagtctcagagaagtttagtgtcttgatcaaagacacacagccatttggtatgaaagcctgaaagattactatgttctactctctatctagacattggcatttgataaaatcgtagacaagaattgttgtctattttggtctggagtgatgtctgtttgtttggttttttttttttgtttttttttttttttgagacagagtctggctctgtcacccaggctggagtacagaggcatgatcttggctcacgtagatcttggcctaggtagatctaggccctctacctcccgggttcaagtgattatcctgccttagcctcccgagtagctgggattacaggcatgcaccaccacacctggctaatttttgtatttttagtagagacaggatttcaccatgttgtccaggctggtctcgaattctgggcctcaggtgatccgcctgccttggcctcccagagtgctggaattacaggcgtgagccaccacatccagctggagtgattagtaacacgtggtcatttgtaggttgtttaaaggaagtggtaaatatctccgttgttttaatcctggaaatgtagcatatttgacaaataagtgaataatcttttaatgtgcaaaaaaggtctatagttcctctcaacacctttctccttgaacccactcacataaaaacagaacagatgcttcctggttaaattgatttatatatatttttgagaaagtgattaaaactgcattgtagacctgcagggggtgggtgggggtgggaattgtaaagagtcgcagacaaggatgggtgttactcagcatcttaaagcagcaaggaggaaatgagtgtgagatatctgacctccaatctgaaatgtaatcacattaacaccttgctgaatattagagaataaagtaatctgaactaaacatttatcactgaatattcttttaaattttggatttatctgaaatctagtcttctgcttaatcatttaaatgctaataaacaagacagagtataatgaagaagcccaggtagttctgtaccaggctctcttgtttcactctaatggtttatcattcaggtaaaggttccatccctcagtagatgcagttggaatggattttttgtttttcttttttttggatacacggggaagctaaaacacaaattgaaaaaaagtcaactcgtttaaccttgggcttcatagcaaggctcacttgagcaggaaggatgttctaatagttgaatttatgtgaaaagttggcatggggaggtaacattaaccaactacatgtcagacccttttacttgttattgcctctaagccccacaaaaaccccaaaccaaaacttggctaatattttccattatttccttgcccctgccccataaaggaactgaaacttataaaaagtaacttacttgaagtaatgcagcagcaaataaaaatttaaatttaaacttagtttagttcgattccatgttctatgtttgttccacatcaggaaacagcatcttgtaaaaataatattccctttggaggaactgtcaccttgatataatttcaatactcaggttctttcctccgaaacaaggctgaaattttggacttggaaccaagaaatttctgaggagttttttctttgaatctttttgccaccgaaataaataaatacataaataagtaagtaggtaagtacataagtaaataagtaaaaaataaaaaatgcattcagtcattcttagcatgatctagaactctgatcatggtattctcttctctgtgcattaccagttgtctgcagactttcaaggctgggctattcagcatattcccacccaaatagctccaacttcaaatcattattactcaagtaactgttaacacttctagtatactagtgtaatccctgatgtataggaaacacctcacatttgttgaatacatttgggaatgcttatttccttgattttagaattccatccattgaaaatgtaccattgaccaaatcacagattttaaggaagggagaaatggaagatgctactaaacgtctagtaacatagacattttgatctaagtcctgatttctgaaatgttagcatatgctttttaaactggggaggaagtatggcacttcgataattgtaatctttcccaaattgcttcttttacctttatttcctgtctagaatttctatcactgcatccattcatctgtcagtatttaatctttctttctttttggctacaaaagcatgattagattttgccctgaaaactgctcgttcccatttgaattgtgttaatttctctcaggtcaactagggggaagttatatggagaaacagtaacagtgctgctttccagtgagattttctgtggatgagtaaaaaatgagatttcatgggttaggaccctttgagaacttgtttaggagcacataacatgtattaagatattcattattaagaatattctttcgatctcatgtatatgacattgcatgagatagaaagatgagaaacctcaggatattcacaagttttatgtggggaaaacttgtgggtataatagtatggtttaaaatgatggataaagtcaaaggacaactgtgtactttatgagctttgaattgatcatgaatgattttattgtgtttctttgccttgttctggggatgaaatctgacaccaagttgaccccttgcatttcctttatagttttcttatcatgtagagaattactggattattagctagctgatgaattccaaaatcagaaacctttggattcactgtaagatattcttgaagagctagactctgcacaaattgaaagaagataattccatacataaatttactgtttttacttgggttttgtgccttttcttacatattatcacagcaaaattattagctaatcctttttaattaatttttgaaatatagtcaaaggtatttggactgtgcctttactactttgaaacattaaagttcacgatatttttgccttatatggtctaatgaataagatttgtctgagactcttactcttagatatgtatttcacacctaatttttttggaaattgagatagtggattattattagggtggaagtatcatattctacttggtacaatattttttggtgaatctgggctcccttcccagctccaaatacaaatgctaaattaccttgtttagatttcttatccatcctgtgcttcgatttcccacttgtctttttaaattctttcttgggattttgccagagtcaactagaaaatgtctcatgtgaaactttgagagaagagacattgtaaaatgcctcttatcaaaataaatatatccagtttgtgattattcacttggtgatttttgtcccggaaatttttaactccccaagagcttcactggctacttggcacaaattctatcaaggcaaattcaccttgtttctaccaaatatccatgtagaaaacacagaatgatttgtattaacctatgcctaaagcaagtaataaagcacacctactccaaaatacaatacaaatttccccaggcaacaaatgcgtaaagaaaatattgctgatttttctattatctatcctgcttcttccttgtataaccatgggtagtatatatttgaaggatggatttttaaggtaggatctgtgctaataccactaccaatataaaagattcatcctttgattataatattttgaggaattataataggaaatgcccccatcatttttatactaagtccatctgtttaatgattaaatttcacctttgtcaaaggtcctagatatactggtgaatttaagcatttattgatataaaactgggatgtagcattcactggagtgatttgcttattttgctggtatgaagcagatttttcaaattcattcagttcggctgtgaaaaacagacctcacagtttctctctgacctttgcctgggccattttcatgggatttagtatttgcggagcaagtgaaagctttaacacttgacacctggcccagcgaaattcctggtgcaggctttgcagtgggggcaaggggaaacattgaagttaaagcccctgctccctctggctgctgagccgtgagcctgctgcctgagatggtcaaacatcttttcacaggtggcttatggcaatttatattcttcttttttttccacagctgttgtgaacttggataattctgtggttgacctggagacccttcaagctctctatgagaatgtgagtaatagaaggaattttatgtgtgagtatataatatgtacacagtacatgtctatgtgcacatataggctctttgttttaaggtcctctaaagaaatttgatccgagtgtagtgaatttgcctaagtaggtgtttgttacaatactttctaggaacatatcaaacaaatgtgtggtctcatggtttgaaaaattgtgaatcaaatcatattgcatatgaggtcattcattatttttttaaatttaaataatagtactgatgtgggggctaacttgagccaagcattgttctgtgcacttcatacacacacacacacacacacacacacacacacacactcacacacacttaaaattttaaaatatgtaactgaaaaagattgaatacattcattccaggtgtatgtattgaacatattcatgctgagttgatatacacattgattaccacaatcagattaacacatccatcaccacccacacttatatataagatctctagaagttattcatcttataactgaaagtttgtaccttttgaccaatatctccctattttcctacctcccacccctgggcaaccaccgttatgctttctgtttctatgagttcaagttttttagattccttatttgtctttctgtgtctagcttatttcacttagcattatgccctccaggttcatccatgatgttgcaaatggcaggattgctttcttttttatgggtgaataatattccatggtgtatatgtaccacattttctttatccattcatctgttgatggacactggttggttttatatcttgtctcttatgagtagtgctgcagtgaacatgaaagtacagatatctcttcaacatacttttcatttcctttggatgtattcccagaagtagaattgctggatcatatggtagttctgtctttaatattttgagcaacacctacactgctttctattatggctgtaccaatttacattcccatcgacagtgtgccagggttcccttttcttcgcatcttcaccaacacctgttatctctctcctaatagccttgccaacagatgtgaagtcatagctcactgtggttttgatttgcatttctctgatgaactgtgatcaaatgctattattatcacccctgttttacatgaggaaagttagagaggttagatgacttactcagggtcacatagctagtgttgtgatatagctggggtttagttcattctgcctgtttctctatactgctacagtttatttcactaggtggcaggacatttagcttttgttatgtagtactttttttttttttttttttaacaaaggtggtagagaaagggagtaaggtattgaaggagaataaccatagcacgatatagtcagtgctattgggaagtgtgatatgagagatgtctttagctagaatgtgacattaaataatattgattcaagtagtaataaagcaggttgttttcagtgcttttgattatatttaaggtaaagtctcagtgtggtcctattttgggtctaatatctttgagtgcttttttttttttttttttttttttactttatttctgttcttgcctttgatctgcctggtatttaagcctctgctctttggggcatggccttagttggtccttaacttttctgtctcaagtcaaataattcttggctctggggagacccatgtggtgggcaggatgcagactctgttccttgatcactcccttgaggactgtgtattcaacctgctcatccactcttgagtttggttttttttttttaacatatgttatatggtggtctcatctgtgagattgagaccgatgtaagacaaaatatgtctcatggtgcattgtacagaatctgtgggccaagtgtagaattggtacccattaagcatggtgagcataagcattcagtgaaactgagtcagaaacaaagattgagagggaagctttcagtgatttattatacagttgtcactatgcaaagggaaaactggaaaaagtaaatcgtgtgtttagtatattcacattccaatgaaattggtatttaaaatataaccattgggaggtacagagagttttcatttctcttgagtctttcatactaaccctcttccaaccaggagggccatatggaagaaaggaggaaatactagaggaaagtaatttcttcagcaggtcataatgatttaagatgttggtgtgtgtgtatctatctatcttgcacatatacccccaccataacatttggtgtatacatggtgtgtgtgtgcaccaatatgtgtgtatgtgtatccagaaatgaatgtcttatttacacttgatgtatttatgtgtagaaatatacccagaaaacacagaacattgaatgtttattttatgatggtttagaatattttatttgggctgggtgtggtggctcatgcctgtaatcctagcactttgggaggccaaggcgggaggatcacgaggtcgggagttcgagaacagcctggccaacatggtgaaaccccgtctctactaaaaatacaaacataagccaggcatggtggcccgggcacgtgtaatcccagctactcgggaggctaaggcaggagaattacttgaatctgggaggcggaggttgctgtgagccgagattgcatcactgcactccagcctggtgacagagtgagactccatctcaaaaaaaaaaaaaaaaaaaaaagaatattttatttgggagtgtttagcctttcaaagtaaagtaatttcacaaaatataaatttatgactcaggacaaactggatagctacccagaatcttatcttacttcatgaaagtagaaaaagtactgtgtcttagtctgtttgggctgctataatagaataccatagcctaaatggcttatgaagtgttatttctcccagttttggagactgagatttccaagatgaaggcactggcagatttgggatctggtgagggccctcttccaggttcacagactgcggtgttcttgctgtaacctcacatggtagaagggacaagggagctgcttggggtctcttctacaagggcactgatctcattcaagagggctctgctctcatggcctaattgcttccaaaaggccctaccttgataccatcatcctggggataaggtttcaacctatggaactgagggtggggcgtaaacattcagcgtatagtatactagctggcactggttaaacttaattacaatttttattgctgctgttctcaaactttaaggttcagactttcattgtttcaggaaaacaattattttgtgagagggtgattcacaattattagtttaggtagtaccaggttagccctctgcagtacaggaacttgaaggatccgttatgaacaataacaggtcattgtgcgtattttatatgccaataatacatatatgcaaattcctgattaatatctattaaaaaataagtcttgggaaggcaaaaatagttgttttatccacagcaatagctccagaacctagagttaagtagtatttggaacagttcttccctaaacgtcctgtatatcaggggtcctcaaccctcaggccaaggaccagtaccattctgtggcctgttaggaactgggccacacagcagaaggtgagccactggtgagcaagcattactgcctgagctccacttcctgttggatctgtggtggcattagcttatcataggagctaggagtacaaacccttttgtgaactgcgtatgcacgggatctaggttgcctgctctttatgagaacctaatgcctgatgatctgaggtagaacaatttcatcaccaaaccatccccataatgccaggtctgtggaagaattgtcttccacaaaactggtccctggtgccaaaaaggttggggaccgctgctgtctatgtaagacattgttgaaatcttatagtactcctttcttcctttcctgggaattttccactttttgctttatgttatctttatgtatgcatatgtcttatcttctcacttaactacaaattccttattagttgaattttttttttttgaggtggagtctcactctgtcacccaagctggagtgcagtggcgcagtcttggcccactgcaacctctgcctcccaggttcaagttctcctaccccagcttccagggtagctgggattacaggcacccaccgccacaccgggctaatttttgtatttttaggagagactgggttttgccatgttggctaggctggtctcgaactcctgacctcaggtgatccacccgcctcgaccttacaaagtgctgggattacaggcgtgagccaccacacatggcctgagtttttatattccatattgaacaatatggtttgtataaaatattcataataattactacttaaacaatgacctgagaatagtaatgttttccttttggtattattttataattgacagaaatgaaaatggttaatgtcattatttacatttcaaatgctctgctttttttaaacatcagtataaaaggaaatctggctttcttaaaaataaggtaatttcagaaatatttctgcagagactcagaagtgatacctttaccatggataaagctaaccattgctatcccagcaataatctgatcccgttatgtgtaatttttcaaacaatggtttaaattcaaacggtgacattctattttagggcaaactgagtagatttgcttccttcttgttgatgtaatttcagacgtgattgcctcaggagcccctggaattgctaaggaagctctttccatttcccatgttaccttagatcttttgagtgtccgctggactgaaaagaagcttgagcttccttttctcagtttatactagataatgcatactgcgatgaacccaagatggacctaagattacagaactagtagtagtttattttttggtgcacaaagcggaacaagtttttccatgttttcagttgctaatagactgcatttttacgcaagactagtaggaataaggtggcgcgctgtatgaaggcacatttgtggtgtaccttggctggaggccttcctacttcctgcagactctccccaaactttacagctatgtaatagctagattaaaataggccactatttcatctccatgtgacatgaatatgtgcttacatgttccatccctcctctatcaccccaaatcacacaagggtataccagaagttgtagttccaactgcatgaaaccaatatggttgctattactaaggggactgggattctaactacctcttttcattcatgctgtatcaggtaagggaaggctaggaacccagtgtaactataatagaagtttcatatgatctctatgctatttaatcccattaaatgactgttgaatgaatgaaccctttgaaacgggagctcagcatggtagaacgtttaggcaacatgtgatgccagcttcttttccatgcatttcctagattcccaacttacaaatataggaacattaagccatctgatttgtatttaccatgtttcctcatttgtcactactgttttctctttgtctaaaaaattctatgacaatctttgtacccccctctgatgttccaatcaaagagctcttctcactttgtactagtttctttctcctatctctcctttgttatagcatgctcagatgcccatcaaaccgaaatgaaaacaaaataaagagacatatataacttaccttcttttactcattgtttctttcattgaaaactatttctagaaagctaactaactgtcatgcagtttgctaagatcagtgctaggtagtatattaggcacatgcatatttggaatcgttttttctcttggatgttgctttttatcagtatgaaatattcttcttcagtttagtaatactctttgtcttgacgtttttttaaactgatatttaaatagctattctattcttttgatacttactgtgtgcatgttttttttttttccaactccatctgtgtactttcaacttccctgtgtctctgtgtttgaattgcatctctcatagacagcatgcatttgggtctgtgttttttattcattgtgataatctttactttcttggagcatttagattgtcaacatttattgtaattgttgttatagttggatttaggtctgtcattttttgtccctctaattcttctttcctaccttattatttacatggtattttttgctgttttgttttaacttttatttctattttttttttttagcttcacctctttgtgtcacatatttttcagttatgctaggaatcacaatgtatattcctaaccttttatactctacttagatagaattttcatataaaatatagaaaccttgactccatatagggctttttgctgcctctcctgctccatttaacttattttgttgtgacgtgagtagcatctgtatgtgattgtatttttaaataattaaacatatttaaatatagtttttttatatttagctacatttcccccatttctgaagctcttcctttgttaagaccaagtttccctccagtatcattttatatcgtttttctttagcctaaggaagtatctgtagtgtattttgaaaacacatcttttcagtgatgaattttcttaattttctttcatcttagaattttttttatctcaccttccttttttttgaaggacattttaaaattggatatagaagtctgggttgacagtttttttctttcagcataataaagatatcccaattacctccgatctaccttgtctctgatgataagtctgccatcattgttactttgtattaatacataaattgtcatttttttccccttctactgcctttaggtttttctttttattgggttttaatcagttttattgtaatataagtaggcatggttttcttgtattctgattgagtttgatggacttcctgattataattgtgatttttcaccacatttgacagtttccatctctcgtccttcaacctgaaactattcttttgtactctgttctcccagtttctctcctctctggcttactaatgacatacatgttacatcttgagtactttttcacaagtccctgagattctgttcattagattttccatcatttttttctttctgttcatcatattgtgtactttcactgattctttcttttgctgtctttattcttttattcaacctctcaaatgaaaacattttatttctaatgctataattttcctagaattttcattaatagttctttttttgagactccttatctttgttaacattgataatttttgttacaaattgaatagttatgttcagttttttaaaaggcacctacacacatttaaaagataaaccagaagctagaaaaagaaatttgcaatacctgggccctcagaaaatcgggacccagaatattgaaaaattcttacacttatcaagggacaagtccaaagaaaaatgaataaaagagataacaaggcaactcaaaaaaagtaaactggtatgtccaataaatacaagaaaaagtgtgtattctcaatagaaagcaagaaaatggaaataagacatcagtaagatacttcttcatctccagtgaaaaagtttgacagtatcaaatgttgaagagcatttgaaaaattagtaactattatatactatattaatataatttggtataatctcaatgggaagcaatttgcaatatctaatataattagtccagtaattacacccttagtcatacattctagagaaactctttcaaaaccagttttccaaccatcagacacattaatgggtccaacacttgggtctagtcagatttgcctgatatctaagtataactatcagacaaatcaacctaggccaaaagattatgaaaattaataattagagtttcaagctactaaagtttaatttctttcacataaaaatgttagctgacacttttcccatacatttttatgccctatatggatataaatatagtgaaaataaaaatagggttatattaccattatacaataatatagaaagataatttctatgtagagcaagatatttgtttttgttattgtctctatgactaaaaaaaaaaaatcagaaaattaagtaaaaagaagtatatttcccttcttattccaggaaagtaactgagccttttgtaccctgaggctgcgctctcctgcctcagatgatctttggggaccttggtttttcttgatcacaaagagaatttgctttgtcttcgtcaagtgagagagactgattataatagttaaagtagctgacgtgctcacatctgtatttcttcatacagctttgagttatggtctagtgtcctgtcatttcatcctgcaggactcccttgagcatttcttgtggtacagatcgagtgataataaacttttccagctttagtatatctggaatgtcataatttttccctcatagtcaaaggatagttttactggatatagaattattggttgaaactttattttgtttcagtactttgaatatatcagaccactgccttctggtctcccaaatttgtgacaagaaatctgcagagattcttattgagcatttcttgtgtgtgaccagttacttctttcttgctagtgttaatattctctctttgactttgtctttcaaaagtttgtttataatgtatctcgatgtagggctctttggattaacatcattttgagttcattaagcttcttggatgtttatgttcacatctttcatcaaattggacaagttttcagccattattttttaaaaatatcctctctgttcctttctctctttttcttctaggacttccataatgcatatttggtctgcttggtggtgtttcacggcttcctgaggctgtcttcacttttcttccatttttctttttctgttctttagagttgatactttctgatccatcttcaagttcatggattcattcttttgcttcctcaaatccgtctttaaattctctttgtgcattattttatgttagttattgtatttttcagttcttttcagttattgtatttttcaccttctttttgatttattttcaggttttctatctctttattaatattgccattttgtctgtacattgttttcttggcttgctttctttagttctttgagcatatttaaaacaaaatttaaaaagcctttgtctttactctgccatcaggtctttttcagggataatttgtattgatttatctttatcctttgaatggtgcatacttttgtttctttgtatgtcttgtgattttctttcttttttattttgaaaagtggacatttgaatttaacaatgtaattctggaaatcacattctccctcttcccaagggtttgctgggtttttattaattgtttttatttgttgttttttgatttttgtagtttatctttgtgccaaggataagcctgaagtgtaaacttaaggtcttctcagggcatttttcagcctgcacctttccctgggcacaagtggtcactttctaattttcccgatatatatacagttgcttttaaatgtcctaatctttaatgtctggctcccaaaaggtgggaaagattaaaaggaatcatggaaaaaagagggctctggccgtttaatcccccagctgtcacttcaaccagataaggaggggcaactacagtggggggacagaggctcaacaatggcctcctgcttctttgtttgcacctgagtgatcagaagcagtaatcagggatcagagcacagatcctttatatttggaaggtgaggacattgggaggttggctcactttggctcctgcaggctgtgtgcaggttgctccagaagcgcgtgcacagctgcctgccacagggccgggagtgggggataggtagctgctgatctgctaagagctgacattgactaaaattaactgtaatttaagctccaagcctttctttggaagttgtaagaccaacagtagaccccaaagtttccaacagattctgctagtacaattgtctagatggggaaatagatccctggcactttctactctgctatcttcccagaatcctttctggggtttctaattttttcattcattcttaatatatttttgttcatatcatggagcatagttataatagctgcttttctaaagacgttgtctgataatatgaacatctgggtcatcttgaggttggcatctgttgattgtattttcctttgagatttagtcagattttcctctattttggcacattggataattttgtatcttggacaacatagtctgtgtaactgtatttaatcatttcttttcatttaacatttaaatgagcttattactctctgtaatacaagatatattcaatctaggttttgctgagaaagaaactcattcatttttgaaagcatgcgtttcccagtattgtgtttttcaaactgggttcagtgaaacactattgttttgtagtctatagtaaaaaacatgttgcataatcaaacaggttaaaaataagttaatcaaataagttaaagttaaaaataaaactgtgcagagccataatgtatgcctaataaagtggtagtttccaagtggaggacattttaaactatattcttccttcaattaaattattaatctagtgaaaacaatagtattacgtggtttggaaaatgctactatagtgcagtctaaataaatccagacttgcagaaacgtagctattgtataatgccaaggaatgtgtctattgtgtatttttaatttaacatagttactattttacttattaccaaataattacatgtatatcccttttatgtatctcttccacttatacttcttgtcataagaattattacatgaaaaggtcatcttagtagtgtagcatcattattgtaataggatataaaccattataaggtcagcataattgttttaaagtcattagtcatacagttttctagactagatatgcttgtaagttttttttttttttttgaggtgaaatcttgctctgtcacccaggctggagtgcggtgacgtgatctcggctcactgcaacctccacctcctgggttcaagtgattctcctgcctcaacctcctgagtagctgggattacaggcatgtaccaccatgcccagctaatttttgtatttttagtagagatggtgtttcactatgttggccaggctggtctcggactcctgacctcatggtccacccacctcggcttcccaaagtgctgggattacaggcgtgagccaccgcacccggctgcttatagtttttattttctttttttaaaatccattttaattttagattcaagggatacatgtgcaggtttgttacaagggcatattgagtggggctgaggtttgggcttccgttaatcccataacccagaagctgaacatagtatccaacaggacatgtttcagcccttgttcctctccctccctccttttggagtccccagtttctagtgttcccatctttatgtgaacccaaaatttaactcctacttgtaagtgagaacatgtgatatgtttctgcatatggaataacagtctctagctgcatccatgtttctgcaaagaacatgacttcattcttttcatggtgttgtagcattccgtggggtatatgtaccacattttctttatccagtccactgttgatggacatctaggtttattccacgtctttgctattgcaaatagtgctgtgatgaacatacgagtgcctgtgtctttttggtagaatgatttattttcctttgaatatatacccactaatgggattgctggattgaatggtatttctatttttagttcattgggaaatctccaaactgctgtccacagtggctggactagtttgcattcccaccaacagtgtataagcatttccttgtcttcacaacctctgtaacacctgttattttttgacattttagtaatagccattctcactggtgtgagatggcatctcattgtggttttgatttgtatctctctggtgtttagtcatgctgagtactttttcttaagtttagctgcttgttatcatcttttgagaaatgtcttttattgtcctttgccactttttaatgaggttgtttccttttttcttgttgattcatttaaatcccttatagattctggatattagtcctttgtcagatgcatagtttgcaaatattttctcccattctgttggttatctgtttattcttgtcaagagtttcttctgttgtgcagaagctctctttggtttaattgtgccatttctctatttttggttttgttgcatttgtttttagggtttcatcataaatgtttgcctagaccaatgtctaaaagagtatttcctagcttttctcccagagtttttatagtttgaggtcttacttttaagtttttaatgcatcttgagttaatttttatgtgtggtaagaggtagtggtccggtttcattcttctgcatatggttagccagttttcacaacaccattgattgaacagggtgtcctttctccattgtttatttctgctaactttgtcaaagatgagttggttgtaggtatgcaactttatttcaaggatctctgttctgatccattgatctgtatgtctatttttgtaccatgctgttttggtaactgtagccttgaagtatagttagaagcccagtaatatgatgcctctggctttgttctttttacttaggattgccttggctattggggctcttttttgtttcatttgaattttagaatagtttttcctaattctgtgaaaaatgatgtttgtaatttgataggaatagcattgaatcagtagattgcttcaggcagtatggatattttaatgatattcttccaacccatgagcatggaatgcttttccatttgtttgtgtcgtctatgatttctttcagcagtgttttgtagttctccttatagagatcattcaccttcttggtattcctaggtatttaattaattaattaattattgtggctattaggtacattatagtttttaaaatattctgtgttaaagagaaaaaaagtcaatgtgagggaaaggatgttgcccaggatggtgggtacaattttaaatatggttgttatttgaaagatgacatttgaactaggaattgcgggagatgaggaagagaagccatgtgtaaatctaagagaccatatcatgcctgaagtgtgagcaagtctgatatattcagggaacagctggaagcccacacagctgaaatggcacaagggaggggaacattgtagaagttacttagagataaattggtggggactagcccctctagggacttgtagaactttgtaagacttgaactttttctctgagtaaaatgggaagttaacacacagttttggctagaggattcatgtttcaaagcttgtccaactcgctttattttgttgttgtcattctgttttgttttgtttcaggcttttagcagcctgaagccatggtgtttagtttcgtgtctagtgataagcagaaaagagggacgaggacggggctttactggcccaaccagaaacagaaactaagaacccacgactgtattctgtcccttggacacccctgtaagaagttctgacaactaggaatagactgtgagaagcaaggtggagtgtcagagacctttcagagttaagagatgatagtggcttggacctcagttaagagatgatagtggcttggacctcagtgtacacagtggagctgctgagaagtggtcagattctggaactattttgaagaatatgccagtagaatttgccagcgggctgggtatggactgtgagagaaaacaagaaattgagagtgatcccacaatttttggcctgggcaataggaagggattgccattagctgataagtggaagcagcttggtagaaaatggggcaatgagaattctgtggttatgttaggtttgagtggtctgttccttcataattgatatgcagacacaggggtctagtagtctcaaggcagaaggtggagagtgagggtatgagtgttagaaagtctaacagtatgggttcaattgggacataaaccacagactaatataagcagaagagtttattataatgaatgattaacctatgatggaagagtacaagaatatagtgataactttaacagcaccctagggatgagtaagcatacccaagttcggataagcttggaagggggtcccctctctaagactggggtgcagatttcattggagaaggtgtgtttgcagcccactggtttgcctgggccatagctggtcacgtctctgggcaagcaagaaaacacacttctgggggcaggtgaactctagctagtgggcaggcatgcaggagttaggatgctacagtgaacgtgaagcccagagtgtacagtatctatgtagggagggcccaggaaacaaacctcagggcacaggcaagccttggctggtgcaggtgcacagtgcgtggtgtctgcattggaggcaaactggtcactgagtctaggtcaggactgcaaggttgccaaagggctctgcactccgggtatgctgctggggtgtggcagagcacccccagatgtcattatacacccacaccgctgaccaattgtgctgcagaaaaggcaaactgcataccagagtcaggaagaggacccccttcctctagcagtgcccctccatcacctcctactggcaaagcataacctcatattcactgtaaagggggaccgggagctgctgagtccagtccgttatctcggattcagttctagaaggcaaactgagggatgagaggcagtaactggcacagatggtagggagggttgaggttttaggaggtatggtctcatctggcatgacagtcacgtgtgtttgtgacttaatatggtaggtgtcatgctcatatgaaagtgtctctagaaacaaaaagtgacatgcacgggcttagacttggcttgagtcctctttggaaatctttccaactttatcatgttggtgtaatggtaattattaacttcaggtctgacacagtgtttgaaagatagctgaatagtcttaattttgccttaatagtttaataataacatttgagggtacatgtgaattttatctagaaaaaataaacatatttaaactcaattttaaaattaaatatatgggtaataatcttatttggcattccacttcttatccttgaagaaaaaaatatcgctaacctaaatcctttcccataaatacaacacactttctttttgccagatgcttaaatgtcattggtctgttttaaagtaattctatttgatctctgtgaaacagatggaagtatttgttatacaaaccttatttagaatgtaatctttttttctctcctgattgcatatgtttggcagtgagctttctcttctagatgtgaatgaatcttggttagcctccaaatctaaacacaccctaatttcaaaaaccacaatcctgaggactcaattcctgtttactgaggaattaaaacctagagaaaccacatagttactaaatgcagtacaacctagtgagtttttaaaaacaaaaatcattacagtttatttagggttattgactgtcattaagttcagagagtaagatcttttcctaagcagtatataatatttacagttggttaactatatctattgcttctgaatcttgaaatatactccttatatggcaaagtgagcttgtgaggtggataggaagcagtagactgtggtgtggggaatgagggagcagatgaggaaaattactcatttttttaagtgtgttgaatgactaacttcaattcaaaaatgtgtctgttaaaatattcatgtataactatcagttgcattgtgtttttctgaaaatcatttttttctttctttcatatttaagaaacaaataaacaaaaaatcttaagtattcaaccagaccaaaacctggacatgaatactattagcagttcaactgaagaaaaatacatgaaaaaagataggaagggaaaaaattcaacaccccctttgtcattaaacagttctaatccaattagaaggaagaaaaaaaagcaggtaaagtagaaagaattagagatctaaatttacacttctcaggattggatttcacaggcatactttatagttttagcaatctttgcttaaagattactagggtagtagttgacataccataaacttgaatctggaagaagggtatcatgcatgccacatgatacagcatcctcaaaagtgtataccatattacattagtgaaaatgaattggattaactgttctgtgttcccataacttcctagaggaatttaattgattcatgttttaagcaacatttaatcatcagtttcttcaaacaaaaatgagaggagtattgactttccccacatagctgttttaaaaccatactctcaaatttcattgttttattgagttggttcatggaatccctgcaagtatgctcattttcaaccataaacgtgcttaaggtgaatttcagaattaaaaaaaaagaaaagctttggatcttgaatcacttacagattgactctataaacttaataaaaaatcagaaattatatttctttcttgttaatctaaggtgtgacactgcctgtcacatcataaaattattgactgattttttgatagttatgcttcttttggataaaccaactatattgcataaattcctggaaaatgaaaattaaaagattaacacagctgcattggaaatatgtggaaataaatgatccgggattgttcttatatgtcgttcctgttattttactttattttattaagagacaaggtctcgctctcttggccaggctggagtgcagtggtgtgatcatagctcactacagccttgaactgctgaactcaagtgatcctcttgcctcaccctctcaaatagctggaactacaggcatgcatcaccatgctggctttctcttattttaatatgatatttttcttttattagttgacatccaaaaggctgacattgtagagcagagagcagtttcatgttcacattgtggaatttatggccatttactttttgtaggcgggtaggttctctgagcacttctccttcacatttgtttttagttttgcatggtccgtccactttaccatcttgtagccctctgctctccagccacccacccccttttagtttctcaaaaccatgcagatcctaccactagaacagcactgacaaaaggtgttccttcaggcaggagatttccacttaccatctccttctagtaaaagtctcctgaatcttcagttgtcattcttttcttccttag

SEQ ID NO: 7 - DNA, 170834 bp, Homo sapiens
  misc_feature (1)...(170834): n= A,T,C, or G
gtcaccatttggaatagagtttgcagtaggaatagattatacatggctttcgtgttgtctcatttaattctccacagcaacactaatgtgggtatttttttctttcaacaaatatttactgagctcctccatgtacgggagataagtattggggccaaccttgttacctttgtcttgaattaccaatcacatttcatagatgaagaaactgaggttcagtgactgacccatagtaagttatcaataaacagagtcagaaatcgggtttaggactcaaaacctttccatcacaccactaggggcatcacacatctccctcttgccccgaccatcacaccagaaggacccacagaggtacatgcacaaaagccttgcatttacagtgtccgactcacacaggtccacacattgagtccacatctcctacttgtactatttgtgaaaatacaactaaatgcaactttgccttgagatctcatctttaaaaatggaatcaaaggcgtctcctattctgtctctgcttccaaggatcaggtgcggtcagagttatacattcagattcctaattcaaggaatgtacttattatataagctgggcatcaatcagcatccatgtattgagcaccttctaaatgctaaacagcaaaccagacaagtgaagggaacatgggcatacagaatggcttgcaatttgtgtaagaattggggaaaccaaactggaggaggccaatactggaggctgctagacaggagagcaatgagctgggtctgaaagcactggatgctgggatgggtgcacaggggaaaagtgagcattccaggcaaagggaacaggcttggcaaaggcagagaggtcccatccattgtggtataaggcaaaggctgttaccagagagggctaagcacgtgaatacccagatctcaaaacacagaaatggaggcaatggccctggagatccataccctaaagaggtatgggctgtaatgacagcaagccttggaaggtctagaagggcacagacctcttgggccccacccttcagccattttggatgctcttcaattctgggcatgaggcctggaaaagttggggagccctgcaacatttccaccagatggcagtatctcctaacgtgactgtcctgggggccatggaggggatttagcttgcaaagtgcctgatattaagatttagagtcccaggaggccatccttgtagccacagttcaggcttctggttcattccctccctccatctcccatgtgcttccaagatttcatcctgggaatggaaactgtgaagctgtctgaggaaccctcttcaattcatgcggaggatgatcactccctctcctttctttcgtggtatcaccctctccccatttgagaacctgaatgatgctggcaggctcacgcttccagtgaaccacaattacttgtaaagcagagcagctggatcccatgtgtgatctgcgttcagttttgccttaacccagtgcaattacccgatgaccgcctctgaaatgtgcctgcctttctatccccttcctgtgagcacacccacctgtgagcacccccacctgtgggctcctgcactgaaccccttgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatttttttattttttaaaaattataaattttggtcttgaactcctgggctcctcaaattcctgggctcaagtgccttggcctcccaaagtgctaggattacagacatgagccaccatgctggcccacactctctttatagctacctagtccattattacatattctcagcatggcgtttttcactgtctgatatttccccttctcttcctccctccttccctccttatttctcttttgacctcttctcaagtagaatgtcaactccatgagacctatgacaattttatctgaaaacctagggcagtgctttgtccatagtttgacactgaatatatatttgctgaataagtaaatgagaatagagaaaatgtcaagaagttgaggacattctcgtctgatggtttgtaattgctctgtgaagtaggaggccaagtaggagactaaaagtagaaatagtggagtgggcaagttgaagagggtggaatttggggtggtgagaatggaagaaagtgatgacaaagagaacacagattaattgttggacagcatttaggccctgccactctaaaaatacaatgattgcatgaaagaatgaatgaataagggcatgtcattctgctgctactgttctgaaaagcgcttctgagaaattgagctaagcttaatcacacagtatgtgtggtagacagaaaaattaccttcaaagatatccatatcctaacccctagaatctgtgaacctgttgcctcacatgacaaaagggacttttatagaaatgtttaaaatttaaggattttgaggtgggaagtttatcttggatagtccaggtagactcgatataatcagaagggtctttattagagaacaatggaaaggaaactcagagttagaggagatctgaagatggaagcagtgggcagagtgaaggaagaaaggtgatacaagcaaggaatgcaggtggcctttggaatacacaaacggtaaggaaatcagttcttccctagagccccaagaaagaacacagtcttgttgaccccttgattttaggtcttctgacctccagaactgtaaggtaatacagttgtgtggtgacttacagcagcaataagaaattaatataccttcagttctatgcagaacgtgcttctcaaaccataatctgagaccttcttaaaatgtagcctctgattcagtaggtttgatgtggtgagacctgagattctgcatatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggcagagaaaaaaatggtgaacaggccatgtgcagtggctcatgcctgtaatcccagcactttgggaggctgaggtgggcagatcacgaggtcaggagatcgagaccatcctggctaacacagtgaaacaccgtctctactaaaaacacaaaaaattagccaggtgcggtggcgggcgcctgtagtcccagctacttgggaagctgaggcagaagaatggcgtgaacccgggaggcggagcttgcagtgagccaagatagcaccattgcactccggcctgggcaaaagagtgcaactccatctcaaaaaagaaaaaaaaatggtgaacaccagctctgttaatcatacagctttggacagtgtgaggagaccaatcacttcaaattgccctcaaaaatattggcaggatagagagttagcagcattttggacttgtctgagaattaaaatgtccttttaatttattttccaatttaaaaaatttgtggtaaaacacacataatgtaaagtttaccatcttaatcattttcaagtgtacagttcagtggtattaaatacattcataatgtgtcactatcgccaccatccatctcctgaaccctttttatcttgtaaaactgaaatcctacactcattaaataataacttcctattactcccttaaccttggcacttggcaacaaccattctactttctgcctctatgattttgactatgctgagtacatcatattagtgcaatcacatagtatttgtcttttgtaactggcttatttcatttataaagtcctcaagtttcatccatgttgtagcatatatcagaattttcattttaaaggctaaatcatgaaggaatagaaaatctgaatatacctataacttgtaagtggaataaatcaataatcaaaaattttccaattaagaaaagccatggacctgatagtttcactggtaaattctatcaaatatttaaagaactaccaacaatttttaccaaacttttctaaaaacttgaagagaagggaacacctcctaactcatcagagtcctgataccaaaaccaaagacactatgagaaaacaaaactacagaccagtagctcttatgcacattgatgcaaaaatactaaaaaaattactaacaaaccaaattcagcagcatactaaaaagattatacactatggccaagtagaatgaattcttggaaggcaaggatggttcaacatatgaatatcaatcaatgtaatacattacattaacagaatgaaggaaaaaacatataatcatctcaatggatgcagaaaaggcatttaacaaaattcaaaaccgattcatggtaataacacttaacaaactacgaatagaaggaaattacctcaaaataacaaaagccacatatgaaaaaaaccccagcaaacatcatattcaatggtgaaagactaaaagtttttctctaggattgggaacaagacaaggatgtccactttcaccaattctaatgaacatagtactagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncccaggagcagaggttgcagggagccaatatcgtgccactgcactccagcctgggagacagagccagactccgtctcaaaacaaacaaacaaacaaacaaacaaaatctgtcaccagagtctaagggaaaggtgattgtattacttgcttactgggattaatttatcagtacctatgacaacactttaaatctttcactcagagattaggaggggaggataagaaaactaatttggaagtctgcctctccttgctcagacaacacatgaaaggttgctgcaactctttagtgctttagtgctcatctagtctttcaggcacttcctagccaaggaaacatcttctagctgtttcttcctctgctccactaaagctcctgggacttcatttatggcagataaaaaacaattaaatataagatttttaaatgacaataaaaataaaaagagcaattgagcaattgggttgtcttttttacaatttaaaatttcctgtctttagacatcctagagttgttgattccgggattcctggggagaaaggctcagggtcggctaacttgccttcatcgaggcaatagttttatgggcctcaggcaacaatggcttagagagcgtccaactaatcaatctcagctgtagggatggaagtgggtgggtgggtacagctcaagcccttggtacaaagcagcccgctagtctctaagatatgttatcttaactcccacaaagtgccaactcccttcaatatttaaagaacctgaggccaagcacagtggctcacacctgtaatctcagcactttgggaggccgaggcaggtggatcacccgaggtcaggagttcgagaccagcctagccaaaatgatgaaataccatctctactaaaaataaatacaaaagctagccaagtgtgatggagggggtgcctatatcccagctattcaggaggctgaggcaggagaatcacttgaacccagcgggcagagcttgcagtgagccgagatcatgccacttcacttcagcctcgtgacagagggaaactccgtctccaaacaaaacaaaacaaataacttgaaagagggcttanaggccgggcacaggggctcacgcctgggtgggctctancactttgggagggcaagtggggaggatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttattattagcgcatgtcctgattgagtgtctagtaaggcttggtcctttcctagcctcccaaaagtaagtcaatatcctcctttatgttctaatagaggaagtacacagtggggagcatgtacagcagtgggttgatccaagatgaccttttctgcttcttgcagtaaatcctaggtggctgctatcaccatcaggcatatgggtcatccctcagcccttatgtccagttgctttgataaacaggcattggatcttctcatattacctggatttggagttaatgtctcaaaattaatctcttgtctttaattgacaaacaggtcaaaaggcattctaatgtctgaaagacctagagcacgaacaatgagtctgtcatttttattcaataacttggaagggcttatggcattcgtgagtcaaactgagaggctcattgtctcttcctttaaaaatcttcctgaaggggcttggctatcagttcaaaattggggatacagatatggcaaaacgctgtcatacctaggaatccatgcaattgtttcctggtaggaggtactgctactctcaccagtgcctcttttggatcaaggtataaactcctctacctttgtgacagttcaaattttaagtatttaacagaattctggggaaatctgggcctttttcttgcagactttagtagccctcttcagctagaaaatttacattccaaattgtattctgtcctgagtcttccttacccttgctggcaactaggatgtgacccttcttttaattggagatccttatatctctgggtaagatttcatcagaagtggtagaagaattcttaaaactttgagagagtacagtccaaccatgttgtttgaccctagtctcaaaatcttcccatgcaaaagcaaaatgccctgagagttggagcttaaagatgtgcaaaaaaaaatgcatccttaaggtccacaacagtaaaccagctaaagtctctagagagaatagtaagtaaagcataagtgttaagcatcagcaggaggatatcttctacaatctaattaatggctgttaagtcccgtgtaagtcaatagtcttgtgtgcctgcctttgtgctaacaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncttacaagaccaaccttttttaaaaaagaattttaaaggctggttcataatatgcaaaggaagactacacatttgcttgctacatttaagttgcacagtataatatatagcttttctccctcctctgggttattttgaaaatattaataagttggatgtctattccatattttcaaagtggttatcaatgacttttacttaaatacttgtaaaacttattaatgccactttaatctcagtttactgcttaaagctttagatatccatatttttttaaaaaaactattcttaaaaaataggtaactaaatttaataaataatgaaaacaatgacaaaatattcaatgaggaaaaatgtttatccccatattgtggccagaacgttaagaaacttttacaggacgaagtctgcctgaaagggtttgcttgggaaagtggtagtttatagcataacatctgattttgtgtgtttgtgccaagggtgtttcctttctgtctttttcctttttataggcagccacaatagtgacatgtttaatattgatgagtttgacctcctgggagttatcgtatatgcaccctttttgaatacaaatgctgtttaatttatatggtaaacacatagaatttggagactagcattttaatcaacactaatctcagtattttctacttctcctacctctccttctgtcaacaacaacaaaaaaatcacaaaccaattttataaatgacagctaatgtcctctcctggagaagcagggtctgtgtgtgaaacataaaagcagcaccatgtgtagtaacctctcctaaaccatatgctcaagcacattaacagtgttcaattagatgatcaattattcaagaaacagaacaatccattcattcaacaaatatttattgaacagccatgcagaggcagacagggggttgtgtatgtggctgtgacccgatggcacctctaaactggaattgggcttcagtttttctccgtgtaaaaatttcttcttttttatttaacttttctcttggcaggtccagcagctttgaagatcagagtgcatcttccagctgacgatctcttcggtcggggacaatgcattcgattcgcacggtggagattaaagtccccgagatagaggaaacgtttttcgcgcccaggttcagcgaggagccgcgcgggggcagagggggcggcggcggcgggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttgtgctttcaccacttagtgtgcaatctcatgtatgctacacactctcgtgtctcaggttccatataacacagagactatgatggctgtgcaagagcaattgccacaggagcaaatattgaagtatggtatctaattaatctagagagcctgtgcatagcaaaagactctatcaagagagtgcacataccacctacagaatgggagaaaatttgtgctatctacccatctgacaaaggtctaatatcgagcatctataaggatcttacacatatttataaggaaatatcacccccccattaaagagtgggcagaggacatacacagatatttctcaagaagacatacatatggcccacagtcatataaaaaaatctccacatctatgatcattagagagatgcacatgtgacccacaatgagataccatatcattgtggtcagaatgtctattatttaaaagccaaaatataagagttactggctagcttgtagagaaaatgacatgcttatgcactgttggagtgtaaatgagtttaaccattgtggaagacagtgtaacagctcctcgaagtcctaaagacagagataccattagacccagcaattccattcctgggtatatacccaaagaaatataaatcattatattataaagacatatggacatgtatgtttatgatttgctatttattcacaataagaaagacatggaatcaacctaaatgcctattgatgatagactagacaaagaaaatatggtacatatacatggaatactatgcagccataaaaaaagaatgagatcatgtcctttgtagggacacaaatggagctggaggccattatccttaacaaactaacacaggaacagaaaaccaaatatggccagttcttacttgcaagtgggaacgaaatgatgagaacatatgcacacattgaggggaacaacagacactgggacctatcggagggtggagggtgggaagagagagaggatcaggaaaaataactaatggacattaggcttaatacctgggtgatgaaataacctgggtaacaaacccccatgacacacatttacttatgttacaaagctgcacatcctgcacatgtaccgctgaacttaaaagttacaaaaattaaaaaacagataataatggttttgaagattaaataataatccatgtaaagcaataatcatcatgaatatacatagtaaatagtaacttagggttctttgaatccactgattacaagttataggaagaaggcatttctcatgatatattcactagtgtaggccactgtctagcagctatcagggtgtctggtacatggtagaggttcaatagcggtgctgagtgagtcagtgaataaatgcttaaagttttatcctaagtcacttggataatattatatgtaaagtaggaacataaaagatattttgtattaaattcaggcttctttctttctttctttctatcttcctatgtctctaaccaaagttacttgtgttgtattcagatgtggaaaaatatcaaatgaggtgatatttacttagaaacttttaagtgactttatctaccagatgtcacaaaggcttagaagtcaaaaaatatctagacttatacctatattaagaaaagtattataacattctttatgaaaccagattttaaaaaatggttgttcccaacacagaaacttctataaagagagtagaaagattaccacaacctaatattttaaatgtgattcaagaaatcatttacacattgataataacatagcctccatgcttaaattagaagtaaaatggaaactcatgaagagagaaatatatgagaaccacttatatgatgagttcctcaaaacctctanaattaaaaatgagaaggatggctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnttatatcagggggcaaaaaaaaaaagacccccatgaacacaaatttcccaactgagtcccccgttatttgtttgtggttgtttgttggctaaacctacaagaggctgagaaagtatctgactagacaggaaagggattgccatgtttatcttgacaaatcatgattgttgaagatttaaaccagtatcagtagagatcaaacaaaccaactgaccaaccaaccaaacaaaacagagctgattttgttaacttgcaaagcaaagtaatatattgccagtaaagaaattcactcattgaaaaaagtcaggactttttaagtaaaagaaaaggggaattggtgcttatgctaattaagcattgaaaaccagcactctggataagtggtaatgagtccatgagtaggctcacaattggttaaagcaagttctattgttcactggtcggaaaagagtgggtccttttttagcttcagctggtctgaatccatcaggataaagcaccataataagttttgataccaccttgctaaaggctactgaaaatggaaatttacttttaaacataattcatccctgggtctggagctattagttaaaaagagaaaggagggtttcttttaaaaacaaagagaaaggggaggaagccgcaggttgatactccagcatgaacttctcacgctcctgtacatcttgctgggtattccaagaacgcaagaccctggccgttttctacttgggccattgctcagatgtatgttggcagtaagcaacatttaggcgtgaggcaatgtcttttctctggggcagagagcaggcttgcttactggttggttttgacttctgaatgttaaatcaatcttgcattcctggtaagggaggagaccacccttcatattgtcttatgcccaatttctgcctccaaagaaagaagaagtaaaaattaaaaggcagaaatggaatccacaggcagacagcccagcactataccctgggcctggtagttaaaaaatcaacccctgacctaactgcttgtgttacctatagatttcagacatcgtatggaaaagcatcgtgaaaatccctgtcctgttctgttccgttctgattaccagtgcatgcagcccccagtcacataccctctgcttgctcaatcaatcacgaccctctcacacagacccccttagagttgtaagcccttaaaagggacaggaattgctcactcggggagcttggtttttggagacatgagtctgccgatgctcccagctgaataaaaccctttcttctacaacttggtgtctgagaggttcttgtctatggctcatcctgctacactgggatacatctaccattggtcaacatgtattatctttttaatatattgttgaattttgttagctaccaaattatttagaaattttgtatctttgctcgtgagtaatatcaatgtaatgcttgcctcatagaatatgttgggatggattgcttcttattcaattttctggaagatttcgtatagaataggtattattccttccaaatatttggtagaattctgcagtgaagctacctggatgtgaagtttcctttgtggaaagacttagaattacaaatccaatttctttagtatatttatggctattcgtgttatcactttcttctggatgtgctttagtagtttgtattgttaaaaaaattaattaggaggccactgggctgagatagctccagcgccttgggttcttatgctagcaaactaatcagaagccaccaactcacttctaactagagactttccactttaaccatcaaatattttctttatcttgcttccaaaaacatcttataaaagttttccccttgttccctctcattagagcccaaagtgatttggtgctgcccaattcatgaatcactatctgctcaaataaactcttcaaaattttaatgtgcctaagtttctttgtcacagtatctttcaagaaatttatccattttctctaaattgtaaaatttattggcataaaattgttagagtattccctccattatacttttaatatttgcagaatctgtagtgatgtcacctttctcctgcttgatattggaaatttgtgacttctctttcctttgctcatcattctggcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaatgtgaatttttatcccttttctgggtgacttatttttattttgctggccagctctcttcttgtgggttggtgcctttgcctgcctcttcgtctcttcattctctgtccccgccatgatggacactgtgaatgtcagtcactgatatttctgagtctaaatggcggcatgatgcgttacgaactgcttccttcgagaaccatctgcaattaaattcaagcggtttagccttttggatccaaaattaattaattaatttatttatttatttatgaattattttttgagacaggcaacactcttgttgcccaggccggagtgcaatggcgtgatcgcggctcactgcaacctctgcctcctgggttcaagcgattctcctgtctcagcctcctgagtagctgggattataggcacccaccaccacggctggttatttatttatttatttatttatttatttgagatggagtttcgctcttgtctcccaggctagagtgcagtggtgcgatctcggctcactgcaacctccgccacccagcttcaagctccgccacccaggttcaagcaattctcttgcctcagcctcccaagtaactgggattaaggagcccgccaccacgcctggctattttttgtatttttaatagagacggggttttgccatgttggccaggctggtcttgaactcctgacctcaggtgatccacctgcctcagcctcccaaagtgctgggattacaggtgtgagccaccatgtctggccttggatccaaatttaaatccaaaattggatttaccaattaaatccaaaagggagctgagtgaggtggtgtgtgccagtagctctagctacttgggaggctgaggcaggaggatggcttaagcccaggagtttgaggtaagcttcagcaacgtagtgagacctcatgtctattaaagaaaaaaaatctgagctgggcgtggtggctcacacctataaatctagccactcaggaggcttaggtggggggattgcttgagcccaagagttcaaggccagcctgggcaacatagcaagaccctatctgaattaaaaatcttagatggaagctgtatagcccataacttcgcctgttgtcttttgaggcaggaggtatttagggggatgagagagcaaaccagttctgcagttttttatgtggtaactctggcctcagactacagagctttctcctagcttgagaggctcctttctgcgctgtcagtagccttggaaacggtgttgttccctggcagtgtttacccagccgctgtcccctggcgagttcagagccaggtgatcggcagtgctcgctcttaatgactgtgtttcatctgcagggcgaacgctgttggagaagctgttcagccagcaggagaacgggcctccagaagaagcagagaagttttgctcccggatcattgccatgggtcttctccttccttttagtgattgcttcagggaaccgtgtaatcagaatgcccagacgaatgcagcttcgtttgatgtaagtaggagaattcacttctgtccgtgaggcagatttgctgtggaaatagcagctacgatgtgtatctgcccagtcaccctataatttaaaaacaacatgtgattcaagcattttttttccttagctgctcaacagctcggtatgtctgtttgtacacaaaaaaaaaaaaaaaaagaaagaaaaaaaactagcgtattaagtacatttgccaaacctccttttcttgacagatggtcactcaagagttctctagcctgctttaatcaacgatgattttaaaaattgatttctaaattatttttaattgtggtaaaatacagacaacataaaatttacctctgtaaccatttttaaatgtaccgtttagtggcatcaaatacattgacgtggttgtgcatccaccaccaccatccatctttagaactcttttttatcatccaaaataaaccctgtacccattaaacaataactccccatcctcctctttccccaagcccctggcaacaaccattctctttctgtctctatgaatctgactatgctaggtagctcatataagtggaattatacagtatttgtcctgtttgtggctgacttatttcacttagcatgatgtccttgagattcatccatgttggagcatgtgtcagaatgtccttcccttttaaggctgaatcatattccattgtatgtatatgttgcattttgtttatacgttcatccatcaatggacatgtgggttgtttccaccttttgctgtaatgctgctatgaatatgggttcataatgagtgaataatgccgctatgaacatgaggtacaaatacctcttcaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaatccccataaaggaagagttaggctctgacactgcctagagaacacacccccagcaggtgcagcaacactgcaagtggagaaaattctcaaaagctgataaaatcatctctatagcacagattgctataaactttataagtatataaatgctagtttctgagaaatttataactctataaagataagggcaggagacgggcaaagtttcttgggctctttctgctccctttcctccctccacccaaataaaagactcagtgggaggggccatcttcctgtccttcaaacccagcctctaatatcattttctgaactagaaaatgcaggcctgtccagatgcagtggctcacgcctgtaatcccagcactttgggaggccgaggcaggcagatcatgaggtcaagagatcgagaccaacctggccaacagggtgaaacccggtctctactaaaaatacaaaaattagctgggtgtggtggtgcgtgcctgtagtcccagctactcgggaggctgaggcaggaggcgcacttgaacccaggaggtggaggttgcagtgagccgagatcacaccactgcactctagctggtgacagagtgagactccgtctcaaaaaaaaaaaaaaaagaaaatgcaggcctaaggatgttaggtgtcttgtcaaaggtcacgttatctttatctaggaaaggcccatgtcattgatcacacatattacatattatatacctaattatgcagaactgaagaaatctagaatagtataaccatgaagaattctctcattcttttataggatggaaagtgttccttattttctgcctattcctttcccctgaaaacacgtggactctgtaccttgacttcatgaagtacacccatgtggggaggcatgggattgcagccatagcttaagcaacagaagctacttcctggatctcaatcccgatgtcagaggcgtttgaaccagagtgactccatcttgaatagaggctgggtaaaatgagaccgagacctactgggatgcattcccaggaggttaagaaattcttagtcacaggatgagtaggaggtcagcacaagatacaggtcataaagaccttgctgataaggctgataagacagattgcagtaaagaagccggctaaaacccactaaaaccaagacggtgaccagagtgacctctggtcatcctcatgttacactcccaccagtgccatgacagtttacagatgccatggcaacatcagggagttcccctatatggtcaaagaaggggaggcatggataattcaccccttgtttagcatatcatcaagaaataaccataaaaatgggcaaccagcagccctcggggctgctctgtctatggagtagcctttctttattcctttactttcttaataaagttgctttcactttactctaaggacttgccctgaattctttctcgtacaagatccaagaaccctctcttagggtctggattgggacccctttccgataacactgatgcctccattatgctaggtatggccttagacaggttacataacccctggtaatcctgtttcctcatctagaaattggaaataatcatcctcattacctccgaggattactgtgaaaattaaatgaaacaatttttgtaacaggcttaacagtacccaataaatgttacctgttattattatgatcaagatatacttcaaatactctctacttgtaaatgtgattaaaaatgttggttcaacttaaaagaaggaaatcataagttggatctaaaagcatttgaaatagtacggtaataggcaaggtaatcacagattcaaatatcttcatccaccaagttttgggtttttttccttaaatttagtagattttaacaacaaggtaatatttaagacactgcttgctagaattcctagcctcaattttttttaaaaaaaagggcttgcatatttgttttactatatttattaaattcacatcaaaaataatattcattaaatgagagtcggggacttattaatcattaatgttctccaattattgctgtactaatcccctcacgctcttcccacatattcttccacactgtagctaatcaaaagtgaagcttgtagaatcagctttgcagtcatacactagctataaatactcctcttaccttgagaagcaaaactcacaagcctagataaacatggacaccgcagttagggggtaagtttttattcaccatcagaacaaatgttgcaatcctcttgcacaatgaagaagtaaaatcaactctccttgcactagggttttattgggcctgtgaccattgacactataaaaagtaaaaagtattttatttgcacagggctatacaatgtaaaaggcatttctgcatctactgtcagctctgtgaagtagggggtctttggggagggtgcttaggtggaactgacacagctgtgcggcccctttgtccttctttcctcctcctttccctctgaaaagcagtcaccgtgggccctgagacagccttcaggatggaaaccagctatgaagannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggtagattgcaaaaattttctcccattctgtaggttgtatgttcactcttttgatagtttcttttgctatgcagaggctctttagtttaattagatcccattcttcaatttttgcttctgttgcaattgcttttgcaaagccattattgtctctgttttatatggaacctgagacaagagaggtatgtagcatacatgaaattgcacagtaagtggtgaaagcagaatttgtatctagttgcagtctaactataatgctgttgagtgaagaaaatgagatgattcctcccttctgttttcttattagctaaagaataaacaaaataaggtatatgaaggttggtagtggaggtactccctaagttgcccagacattctcacggattagtgagtaataatcgggataatgtgggcagggaggagggcagtatttgaagtggagatctatgacgactcagtctctggggacagtgctggatcaacttgattcttcaccttaaagaagaccttgtcctccacagcatgactttcaattaatttctcctccccagcattttgcatttcagaactgagaagtgcttcagggaacaaaatttctatggatgcattaaacataagaaaatcaggctgtgggacacttagactgaccatctgaagagctgtataaaaccctggattcaaacagggaagggccattttctttctttctttctttctttttttttttttttttttgagactgcattgttcttgagactgagtctcactctgtcacccaggttggagtgtagtggtcaatcctggatcactgcaacttctgcctcccaggttcaagcaattctctggcctcagcctcccgagtggctgggattacaagcgtgcgccaccaggcctggctaatttttgtatttttagtagagatggggtttcaccatgttggccaggctggtctcgaactcctggcttcatgtgatccacccttctcgacctcctaaaagtgctggcattacaggcatgagccaccgtgcccagctgggtcattttctttttatgtattttttttttttcagttgaaaatctgagattgaaaaccaaattaatgaatggagaaatctcacatgcttcatgccaagagtcactcattacagagcaggggaaagccttcctttaattgaaataaactatcacaagtgaaaaagagtattggaggagagtttatttatgtatctcaggtgaaaccatcacccaccctgtgcaccgaggctgtcaagcttcctcaggggacagcactccatctgcctcaagtgaagttaatctcctttctatctttttaaaactaaggtgagcagatgtttcttttcaaatcaacatgtccttagtctttgacttacatagcgagccacatactgccgagcataaaccacttaacaccaaaaggaaactacatattctattagttcaatgaaacaaagtggaaaataagctaaatgcagagctttttattaccattaaatagttgatcaaaataagcagagttaaataaaagggacaaacctgagtaaataaagttttcatttctgcccaggaatataaatatatatataaatataagatagaagtcatgttgattattccagcatttcccaacagatagttgaggaatttctttccaatatttttattgcaccacctggtggaatattttccatatgattgatatttgagaagcaaataagcgctgatttctcttcatcttttttgtgttttacttagaaggttggttaaattagggagaatttcatgttgctatttctactctcttttatgaataacttgccttgcccttcagttaagtgttcgtagagaagactatctaaattattcactgtggaagtacacatcatcacatgtgatgggatattgttaatcgttaaacaagaaggaggctctggtgcatgctaccacatggataaccttgaaaacattatgctaaatgaaataagccagacacaaaagtgcaaatattgcatgattccacttatatgaagttcctagaataagaaaattcatagatagaatgtggcctggagtttaccaagagctggatatagtgggggaaaggggagttaattgttagtgggcacagaatttcactttgggatgataaaaagttctgaaaaagattgtggtgctatttgcataacagtgtgattgcactgaatgtcgctgacccatacacttaaaaatggttgaaatggtaaattttacattacattcattctatcataatttttttccaacttttattttactttcagggtgtgtatgtgcagatttgttatgtgggtaaactgtgtattgtgggggtttggtatacagattattttgtcacccagttagtgggaatagtacctgacaggtagtttttcaaacctcattttccctccaccctcaaggtaggcctgggtctattgttcccttttttgtgtccatgtgtacttaatatttagctctcacttataaatgagaacatgcagtatttggctttctgttcctgcattaattcacttagaataaaggcctctagctccatctgtgttgctgcaacagacataattttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctcttgaattgcttcctgagggaagccaagaacccatgttttctcccaggcagaaccccagttttggggttggctctgtgacaagactgtattgtaatagcaggaggactggggaaaaagatgagaattcaaagaaccaccaaaccagcaggtttaccagttttgttcctccagtgtccctcaacctgaactcaaggtatcccaaaacccagaagtgggcatcagcaccgacaaggaaagctttaaggggcaatttggcaatgtgtgtcagtgatctttaaaaattcacccctttgacccagaaattcaactttataaaagctattctaggtaaatattcagaggtatcctcagagtgatgcatcatgggagtattatgatgactaaatgagataatgcatgtcaagtatttatcacagttcctggtactataacaaatagattattaatagtaccaaaattcaaaggaactaaaatgtccaacaatacagacataaataaaaaaagatagctttatacacatgactgctaactatacagccattaaaagccatgctattacagaaagaataatcacttaattgatttaaacatattaaatatatttaaagaaaccagaaaaataaaacatcagctatcactgaagaatgctagggcactgataacagaataggaaataatgaggcatttatgctgcctttctagaaagaactgtatttcagggtaacaaccatacctagtgaataaagtcttcaatacagaaaaattccagccaaaatgtgcaaaaggaatgatagaattggccgggcgcggtggctcacgcctgtaatcccagcactttgggaggccgaggtgggcagatcacaaggtcaagagatcgagaccatcctggccaacatggtgaaaccccatctctactaaaaatacaaaaattagctgggcatggtggagcatgcctgtagttccagctgctctggaagttgaggcaggagaatcacctgaacttgggaagcggaggttgcagtgagctgaaatctcgccattgcactccagtctggcgacagagtgtgagactctgtctcaaaaaaaaaaaaaaaaaagaaatgatataattagaaaatcaccattttgcaatccctaatgaaataatgggttcacttaaagaacagcaagtggatggaaaatccattagagaaaaggtcaattgaaactcttgcaaagaaaggatcaagctgtcagccccaaacccagggatcattcacagcttcaggacaactgagaaagccagacatggtgggcccctgctgggggtgatcataaagtacacagcatcccccatcgagctttcctgctgtcccccagcctctggaacaaaagaaaggagaactcagcatgaaccaattcaactgtcagattgcacctagcatccattctagagtatcagagagttctgaggatggactagttaccaggtaatattgaacaacttactctatttgctttcaatataaagaatgacattgtagcatactttaaaagtccatttttgagatacatatttgatattaaaataacatgatgtctggagcttgttttaaattatttcacaaaaaagtaggtggcatggggtagaagtggaaaaaaagggttgataaaactattatggcattgaaaatattgatgattactgaagctgagtaatggattcatgggaattcattgtaattctctgagtgtgtatattccactttcacaataaaatatcttaaatcacactattaaaaacatttacttacacagaaatatgtgacacaaaaatcatgtgaaaattcaagatcacaaaactaaacaaagcatgttcctgattttgcaaaaccaagagatacgaacacagtttattaaagagtcaaaatcgctgtggtgggaaggcaaacagtttttgttatttccaggacacacactccagctggactatactgaactccttttctgttcaaggaatgtaatttgaccaaccaggtcagggggagtggacagagacatgaacttttatttctcttcctgcatttgataattgagaatttaatcaggagacactcacctactaaataggattaggatgccttgaatctaaactatggagaatggactgccctgaagccttaaacacaacctcatattttttttttttttttttttgagacagagtcttgctctgtcccccacgctggagtgcagtgtcgtgatctcggctcactgtgacctctgcctccccggttcaagcgattctcctgcctcccgagtagctgggaccataggcgtgtgccactatgcctggccaatttttgtatttttagtagagatggggtttcaccatgttagccagaatggtctcgatctcctgacctcaggcaatccgcccacctcggcctcccaaagtgctgggattacaggtgcgcaccaccgtgcccggccacaatcttacttcttaactgtgcaaactnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnggggacaatgcaccaaagaaatcagcagtttacaaatggataactcattttaagaagcaaggaggcggccgggcgtggtggctcacacctgtaatcccagcgctttgggaggctgagctgggtggatcacagggtcaaaagatcaagaccagcctggccaacatggtgaaaccccgtctctactaaaaatacaaaaattaactgtgtgtggtgacatgcgcctgtagtcccagctacttgggaggctgaggcaggagaatcccttgaacccgggaggcggaggttgcagcgagctgagatggtgccactgcactccagcctggtgacagagcgagactccgtctcaaaaaaagaaaaaaaaaagaagaagcgaggagacaacatcggagctgaaggccacagcagcagaccatccacatcagtttgtgaggaaagaatttatcttgttcatgccctgatggttgacatcagaaaaaaatagccaagaccatggacatctcaattggttcagcttcacaattctgacggaaaattaaatttgagcaaacttttcactcaacgttgccgaagcctttgtgcccagatcagctgcagacaagaacagagctttcaactgaaacgttaaacaagtgaaatcaagatcctaaagcatctcctcaaagaattgtaacaggagatgaaacacggctttaccgatatgatcctgaagacaaggcaccatcaaagcaatggatatccagcagtggtagtgacccagtcaaagcaaaagcaggggctgggagtggtggctcacgcctatagtcccagctactaggaaggccaaggtgggaagattgcttgagcccaggagctaaagaccagcctgggcaacgtaatgagaccctgtctctgttatataatttcttaaaaaataaaataaataaaaaagcaaaaccagtcaagcacaaaggtcatggcaacagttttttgggatgctccaggcattttgcttattgacttcatggaggtccaaagaatgataacatctgcttattataagcatgttttgagaaagttagccaaagctttagcagaaaaattcccaagaaagcttcaccagagagtccttctccaccatgacgatgccaatgctcacttcttctcatcaaacaagggcaattttgccacagtttcaatgggaaatctttagacatccaccttccagtcctgatttggctccctctgacttctttatgtttgctaattttaaaaacatatttaaagggcattcattcttcttcagttaataatgtaacaaagattgcattgatacggttaaacttctaggacccttagttctttagggatggactaaatggttggtatcatcacttacaaatgtgtcttaaacttgatgaaacatacattgagaaatacaatttatatattttttaaatcttttatttcaattttccatgaactttctgaagtcctcttgtgtttagctttctctggttggtcctgagttggatggggtcaaggagagagggaatagggagggaagctggcagtcattgaccattcaacagccctgaatgttctgagtcaatgctacagaggttgtggtttggttgacatcctggactggttgcttcagaggttgtgggtcagttgtcatccagggtctggccattgtctgtttgtttattctgtctctcatgaaaacttttgtctctaaggctctgctcacctttagacgttcatttcagcatagcacagttcctctttccccacctccaatccacaccgccattagaaactccaaatatactgacggatttatttactgtttttttgtttgtttgtttgtttttaatgtgctgtctacttcttctcgtctctggggttgccattaaagctgtctggaaaacttttctgctcccctccgccacccttgcattccgtgctaagttctacccaacttcagattgcaggttagaagtcacttcatccaagagctcatccgggacccttcaaatttctcgaatatgctctcagagcccctaaccttggcttcaatgatcacatttatcacatgttgtggctcagaaaccaatacccaaccaataccccaaaatatagcactttgacatgttgaactgaagaagaagtctcaaagtctatctgatcttccctgttcccctctggccccaggtctgtctctcagtcctctgtctctcccaaaggatgaggttgttctccttatccacctaaagtctggacctctcgtcccttccctgagttttcgtcactgaactcattgctggaagaatgactgaagtctgtcaacacatctggacacattttcgtcacgaaccattgtctgctctgcgggccccacagacaatgttccagaccattgtatgtgattcaagcccattaaattctccgaaaaaatcacttactaccccattaaaatcatccatacttccccatctccctttcacctaagaagaagggtaaataagcatctatcgccactgcataatggggatttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgggaacccacctcttgcatcagcatgacccggatgtgagacatgcagtcaaaggagataattttggagctttaagatttgactgccctgctggattttggacttacctggggcctgtagcccctttgttttggccaatttctcccatttggaatggctgtatttacccagtgcctgtacccccattgtatctagaaagtaactaacttgctcttgattttataagctcataggcagaagggacttgctttgtctcagatgagactttggactgtggacttgtgagttaatgctgaaatgagttaagaccctgggggactattgggaaggcatgattggttttgaaatgtgaggatatgagatttgggaggggcaaggggcaaaattatatggtttggctgcgttccccacccaaatctcatcttaaattcccacatgttgtgagaggaacccggtgggaggtaattgaatcatggggtcagatttttcccctgctgttcttgtgacagtgaataagtctcacatgatctggtggttttaaaaggggagtttccctgcacaagttctcttctcttgtctgccaccatgtgagatgtgactttcaccttccagcatgattgtgaggcctccccagccacatagaactgtaagtcctttaaacttctttcttttgtaaattgcccagtcttgggtatgtctttatcagcagcatgaaaatggactaatacactgccattatacttctttcacagatgaggaaattgaaccttgcagagattaagtgatttgttcaaaatcactcaggcaataaatggcaaagtcaagatttgaactcactttctctgaggccaattctccctacactcttccaagctacttttaaaaagtatcactaaaaaaggcagatgggagcagtggctcatgctgcactttgtgaggctgaggcaagaggattgcttgaggccatgagttcaagaccagcctggccaacatattgagatactcatctctacaaaaagcaaaaaaattagccaggcgtggtggcctgcacctgtactcctagctactaagtaggctgaggtgagagagtcacttgagcccaggaggtagaagctgcagtcagctgtgatcatgccactgcattccagtctgggtgacagagtgagaccctgtctcaaaaaaaaggtaggtgggggggatttaatttattatggtagaatgatgagagaagaagtagcaaatggtttaaatagacatttttatagtgattttgggggacagaaaaagaatggaagctggatacctatcagaaacatggtggcaggggaagtgtgaagagaacaagagccccacctgttcagtgtgaagatgctcccaaggtgatccattaacctctgtttccgcattccacaccactgcttggctcacaccctagctacataaataattgcaagaatcaaatagttcttaaaagatattatataatgctccattctgttatattgaaaattggtgataaatggaagtataaaaatattaacagacttcccaggattgtagatggtgtgaaagtaggatgatagattctaatgcataaaactatgttaccatttgcccttatcaataaactacatggcttcctatttttttgtttgtttggtgttttgctttgttgttgtttttgagacaggatttcaccctgttgcctaggctggagtgcagtggcttgatcccagcttactacaaccttgacctcccaggctcaagtgatcctcctgcctcagcctcctgagtagctgggaccacaggcttttgccacccattccccaaccccctcccacccccctcccctcacctccaccccccagtagagactcggtctcaccatgttacccaggttggtctcaactcctgggctcaagccatcctcctgcttacagttgtgactgtatgttaccataccctcaggatctcttaagtgagttgtgaaaaatattcaggattacctatagaaggccactgaaattgactcaacccataataaccttttccagtggcaagaagttgcaagaggagttctaaagacaaaatcaataagaaattaaggttgaggaaggctatccttggccttagagaagtgctctgcaacagaaatgctatataatatgaacctcatgcataattcaagattttctagtagccatatttaaaaagggacaaagaaacgaatataattaagtttaatactatatttaacccaaaatatgcaaagtatttttatttgaaaaatcgataaaaattattgagatacattacatgcttttgtcctaagtgtttgagattggtatatattttacactaacagcaacatttccattcagattggccctttttttttttttttttttttttgagagaagtctcactcttatcccccaggtttgagtgcaattgcttgatatcggctcactacaaccaacctgtgcctcctgggttcaaatgattctcctgcctctgcctcccaagtagcggggattaagtcacctgccaacacgcccggctaatttttgtatattttagtagagacggggtttcaccgtgttggccaggctggtctcgaactcctgacctcaggtgatctgcctgcctcgggctcccaaagtgctgggattacaggcgtgagccaccgagcccggccggattggccctatttctttaacgctttagtgctcatctaaagctcatctattgcttgatgaccacatacggctaacgactactgctttggaccctggaactttagccttagatttttcaaagcttcttgagcaacacttagaacctgcaatgactgatcccaactatgttattgagttctaaaacagtattatgtgttttgatgcacttaaaatgaaagttatatgtaaatacatacaaaagcaccataggttttcacatgcaggcacaaaatttcttatttaacaatacactgtggaaaaaaatttccaaaacttttttcagttattcagtttaatagagatttagcagaactaagtctttgcttttgacagagcttgccaccactaaaataaagaatttacgttccaacagggcagggcatggtggctcacacctgtaatcccagcactttgggaggctgaggtgggcagatcacttgagctcaggagtttgaggccagactgggcaacgtaataaaaccctgtctgtaccaaaatataaaaaattagtcaggtgtggtggtgcataactgtggtcccagctactcaaaggcagaggttgcagcaagctgagattgcaccactgcactccagcctgggtaacagagttagactcccacctcaaaaaaataaaataaataaaagaaaaagaaaaaagaaaaatttacacaacagaaaggaccatgggaagggaaggagaaaagctgattgttgtattgttaccagaaaggggtcctgatccagcccccaaaagtgggttctcgggcaagaaagaatttggtgctagtccatggagtaaagtgaaagcaagtttgtaaagaaagtaaaggaataaagaaaggctaccccataggcaaagcactaccgagggctgcaggttgcacatttttatnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttgagatttgacgaaaatggtaatcttgtttcagatcttcttgaagatgcctgtagagggaggcaatttgctttgatccagctaccttaatagtgagtaagtgtaaatttgaagcattttgtcacactctgctatctcacttttacctgaaacttcgtattgtgtttagtcagaatttattgagcacctactttgtgccaaagcaccattccagacattacaaaatcaaatatagataagcggttgtccccgtactctcggtttttactgtatggcaaaaaagaaataaatgtcccccaaaaccccaagccatatattgatgtatacaattctagaagaaattggaaagattaaatgtgtggttgattaactctagaaaaagaggacataaaatataaggggttttgttttattttgcctcagaggcaattttgtgtataccaaaaagtctactgagattttttaaaattaatgttcaatctactaaagttacaaattcaaaatcctagttcttcccaaagtttttaagtgcgtaatataatattgtattcttatcttattgtcattgttcaatgtcacttccacagagcttgttattgttaagtcttggcctcatttacaaattttatttctttaaaccttaagctgcatttacaaatgtagtatattccatttccttcaaaagtattttgacctaataaacaaaacctctctgagtgctagataattcttataaatgtaatacattgaacttataattacagtgaagttcatatccatgttccaactcaggaagatttaacttaaaatcacaagtctaaatcaattaatccacctctcattttagatttatagaagatctagtgggccatcttatgttacacgttagaataaataagaaaggaattcttgcaaattccttaacacagatatattaatagtatggctttgattctcctacacatttagatttgtcctgaaactgaaaagatgcttcaagacagttcgacgtgtcaaggcaggttcattgattcaccattgtggtacaggatgttgacagcaaagaaggttgggggaccaggggttatattgggaactctctgtattttgtattcagtattgctgttaacctaaaatggcttcaaaaaattatctattaaattaaaagaaagagagtgagtatgagcatgaaagagttgcaagaacccaaaaaagcaagaaaaaagttactgtctctgttcatgggagagttggttgggctgctacattatcatccataactctcaacttcatttatagttgcatgtcaatgactccacagcttcaagaggtaggccaagtccctggatttcagttacacttatcacctaactctggagtctcttagagatccctatagtctatcaaattggtcctttgattagattttgcaagactggctcttaagataaattaatgtttaccttttaaatgtaaacatctctgaaccttactaaaaccctttggtattttacatgtttcataggcccacacaaattagatattgaaagtgtgtttctttgaatcctgtgtgttggacaaggttttctctgattggtaattatactttacaagataattttagtaacggtggcctccaactctaaaagcatagagaagagaggtaaacaaataattatattatatggtgaactattcagtcatagaagatactcacataagaaatgccaatagggaatgaagtgtccaaagaatatagaagagtttggcacagaatagtgccctatgtgttgtagagtacgcagtaatcatttattgagtgaatgaatggatacacgaatagtgaataaagtgctcatagaagaatactagcttgttagtactagaatgtaaagttacaaggtagggttatagattttgacttatagtaaaataaaacacattccacttctcctaaaaatcccctgcgctccacctgtttatctgccacacccccgattcccagaacccctggcaactactgatcattttactgtctctaaacttttgcagaattttatgtgaatatctgttttcaaatcaattggataaataagattccttggatcatatggtgagactacacttagctttgtaagagactgccaaactggcttccaaaacggctgtgccacatttgaattcccgcctacggtgggtgacagttcctgttgcttcgcatgcttgtcagtatttggggttgtcagtgttttcgattttagccattctaataggtatgtaatggtacctccttgttattttaatttgtgatttcctgaaaacaaatgataagcatctttttcacatgcttatttgccatccatgtatcttctttggtgaggtatccagattttttggcccattttttaactgggctattttcatattgttgcattttaagagtcctttgtatttttggatacaactcctttatcagatatgagtttggtaaatattttcttgctgtctgtcacttgtctttagattttcctaacaatgtttttacagagaagtttttaattttaatgaagtataacatcaatttttttctttcatggaatgtaattttgcagttatacctaaaaactcacagtcataaattttttgtgactaaacccaaggtcatataagttttctcctatgtcttctatctttttttaaaaaaaattattttaaaaatagaggtaagggcttgtcataacccttagacagggtgggtttgaactcctggtctcaagcagtcctcctgccttaactgggatgagaagcatgtgccactgcgcctggaatatatatatatatatttcttttcttttttttttttttttttgacggagtttttgctcttgtcgcccaggctggagtgcagtggtgcaatctcggctcactgcaagctccgcctcccgggttcaagcgattctcctgcctcagcctcccgagtagctgggactacaggcgcccgccaccgtgctcggctaattttttgtattttttagtagagacggggtttcaccgtggtagccaggatggtctccatctcctgacctcgtgatccgcccgcctcggcctcccaaagtgctggaattacaggcttgagccaccacgcccggccattttatttttaaaaaggttcatgattttatggtagggaacaagtttgccccacagaggcaagatttgtccttggttggcctgtctcctggaaagagaattgaggtctggaactctaggcctcttgtccatttactacaccatccagaacatttatgtaagcatttccagcaattgtcctgtccattctgatttgattagcaaaggtcacatgtccattcttaaaatagtatttctgtgctaagtcatatattccactccagcatactgggcaggggtggagtcagcttccccagaagcacattgatttccacatggaaattgggctgtgttctaaaatgctgggaaagcagtcacatatgtgcaatgccagcatgaaaccaaagggttgggggttagatcattaagggctttatgtctaaacaggtgatggggtgttttagttcattctttcattgctacgaagaaatacctgagactgggtaatttatgaagaaaatatgtttaattggtgcagggctgaacaggaaggacggtgttggcactttctcagcttctggggaggcctcaggaaatttacaatcatggcggaagacaaagtaggagtgggcacttcacgtggctggagcaggaggaagagagagggaggcggaggtgccagatctggtcatttaaaatgatctggtcaaaacaacaaactcactattgtgaggacagtaccaagggggtgggaataaaccattcatgagaaaccacccccgtgatccaatcatctcccaccaggctccacctccaacactgggggttacaattccacatgagagttggtggggacacagatccaaaccatatcagaaagtaatggagtcatcttacagaggagtagtaaggtgttagatctgcatgttagaattttattttctgtagcctggaagggattcaagctgtgcatgccttgaggtagattaatgagggagctctagcagtggtcctggagagagaagctgagggctttgaactcaactatggctgtgacagtggcaaggagaatgggaaggagagagcaaattcaagacatgtttaggaattaaaacaatgacaaaaaaaaaaaaagacaaaaaaagcttttcgagctgggtatggtggtgcatgcctgtagtcccagctacctggaaggctgcagtgggaggatcacttgagcccaggagtttgagtctagcctgggcaacataaggagaccttgtctctaaaaaaatttgttttaaaataaaaagcgttttgaatgattttcttgtttctctaattgataatcccacaaattggggaataaaggatattagaacaggaggctggaggtaggatggccacagttgatagattatcttgaacatgttggatttgatatgtctttaagtcaacctataggaaatgtcaagtaggcaagtggatttggggatgtattatagttaggctgagggcagagatagaatatcaccattaaaaccaaaaacagaatttgagtagactaaacctcttagtaaaagattgggcactaatggaaccacccannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntttagtataatttcttacccactgctatattagctaggaaaaaccaaaaggtgtgttaaaggtgatgtcttctattgtatatctaacaagaggtcctgcagaagaggaacgtaatgttaaatgggacgcggtgatgtggtcttgggttgggcttcttagaaacaaagattgtgtgattattcagagcgagctctcaggtgaaaggtaaggggatgaggaaagcagaatagggcaaggaaagcaataggtgataaagtgggttcaggagcttcaggctgatctccatggagagctctagagtgtggatggtgtcatgaaatttttctctcttgggcaagaagccaggctcctgccttctatcagtcagccattggctatgggctgcaaccttccagccatttctaggtgagcccataggattttcaggagaaggatgctgttgtgagctatgagcagctagcactcacaacagcacagggatggtgacttggtctgataaggggggcctgtgctggtaccaacagtgtctaatattgttctgtcctttggaccagtcccatctactcccttctcatattcacttcactccatttcaagcatagattttccaggattctggctgatgacaatttctgggaatcttacaagaggagggaagagggtcccaatactgcaactgaccccaatcattttctctccctaccacctgttctgcattcctctcacccttgtttgacactttcactgaaggatggattcactggctagataaatggctgggcatcagcagcgtctgctacagatgtcaggaaggatccaggctttttctatccttctatttcgccatcattggcgttttgaatttcatcttcatgcttgtttcttcatgaatccaggatatttgctgcagctccagacattaattttcctcacaggcaggattagaagatgggaagaaactagctttccttttactgagaagaaaaacaactttcagaagcctccttttcatccacccttcagacttcctgaatttttcgcgttaggattgggtcacatgactgcatctacccataggagaggcagagaaggaaagactacataggtagcaaaagggaatgtgtcctaatagggagaaatgggcttaagaaggtgctacttgattaagcatccactggtgtcttcctctttcactatgcatcatttgcatacactttttttcccatatatcacattatatgggaatataatgtgatatattatatttatttggaggattatatcaatcctccaaaataaatatgcatttacccctggccctagagaataaacccccaaatctcatccagtttctacattcagcttgaagccatatatcactggtgataagcaagtctggatttggcttcttatgtgtggtgacctgtaactaaaagaccaatttgtctgacatggagagagttaagcacatgccaccccaaaatatgcagatttggcttactgcttatttagagctgaaaatactagagaaaacacacttgcagaaaaggctgtcagacttgccctttcttgcctgttgtaagccctaaaaattcttctgagaaagatgcctttcctgtaccaggatgagaaaataacccttatcactagagactgggaattgatctgcaatagatctgtacaaataaacttcctaaaatcacccttaacttccgctagctttacaccctcctttcccttgacttccccacaatttgctacccctagcccaaacttctttgtcaccatttcacaaatttattatttctactaaaaacaaaaggtaaaaaagctttctactctggccatttcttaggagcttcactgttttgtgtaaggcgccctgtacatgtaaagattaacaacacttgcgtgcatttctcctgttaacctgtcttaaggttatttaattcttagaccccattgaagatactaagggagtaaatgagaatgtatccttcttcccaacactgcaaacacacttaaaatacagtgagggagaaaattagaaatccacagtgaagttttggggaaaaggggaagtagaaaacaactataggccgggcgcggtggctcacgcctgtaatcccagcactttgggaggccgaggcaggtggatcatgaggtcagcagatcgagaccatcctggctaacaaggtgaaaccccgtctctactaaaaagacaaaaaaatagccgggcgcggtggcgggcgcctgtagtcccagctactcgggaggctgaggcaggagaatggcgtgaacccgggaatcggagcttgcagtgagccgagattgcgccactgcactccagcctgggcgacagagcgagactccgtctcaaaaaaaaaaaaaaaaaaaaaaaagaaaacatctatagcagtgcctgctgagccatattggaacctaaggccaaagggggaaataaaaatcagtaatacgggctgggcgcagtggctcacgcctgtaatcccagcactttgggaggctgaggtgggcagatcacttgaggtcaggagttcaaggccagcctggccaacatggtgaaaccccgtctctactaaaaatacaaaaattagccaggcgagttggcgggtgcctgtaatcccagctactcgggaggctgaggcaggagaaacgcttgaacctgggaggtggaggttgtagtgagctgagatcatgccattgcactccagcctgggtgacagagcgagactccgtctcaataaaataaaataaaataaatcagtaatacgatgcatatataaaattttgatattctgttcatcatagattttttgcagtcacttagattttctaaatattgcattaaaatattacttatcttaattaccgagtgttttagcacctgcttgaactttgtgccccagatgagtgcctcatccacctcaccttagctccagtcctgcctatattaaataccctttgcaatgtaatagcttccccaatgggccaatctcatttgtcaatgttggtttctgctctttaggaaaattctttcccattattttccatggcccttcagagacagacctgtattggtaggaggtaattcacggtggatgcaatgcatttatgatcccattctacttggtgtaagttctgagactaaaaagttgaatagtcataaactaactgcagccaggcctcacgtttttgtcacaatacagttctctcaaaaatttaacgtttttctatttgtttctaatcaatttcatgtcctaattatcataatcaaagattttgtctacacacagtttttaagtccaatgagtttcctaagcactagtcttgaggctactcattcttctagagttaaatggtctctgcactgcagcaatttgaaatactatcctctacttaggagatgctaagcttaactgcatttttctggcgagcgtattccttttatccttccaagcacgtttaatgaagacattttcaaaaaggggtgagtctcaaacttgtcaataatatttgctattaaacccataacttctgtgctggtgcttgtttagatgttgggtatagaagttggctgttccaacccaacaagattacaatgatgggacaactcagttaaattcatgcaacagattacgatgaatatctcttttagcaaagctgcagtttctttcaagccaggcacctccatttgaactttcaacctctcttgtgggacttcactgaaagacacaggatcaaggaacatttgccaatattctgagcctggaatgtttgagttctcactgtcctgatatacctccttcactcattcatttctttattttaaggactgtcactaataacacctcaattccaaggtccaagtttttattaagctgttctttagctctgcctctggctaactggnaatttgaaaatgactcaaagagcagttgtctctgtgcttcacctctctctgcttggtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnataattgaagcatgagctcagctcagagagctaagagatttgtccaagatcacatagcagcagaaagaatactggaacctgcaaatttagaccacaatttttttataacataaaacactgcatagtttactcttgggggaccggtaaggatatcataaaggcaaactacaaagctttgccaaagggttcttgttatttaatagctatttagtattcctaaggatgcctgaaacatggcctaaccttaattccatttgatcaggtaagaatatcatatctgtggttgcaaaactagatttaacagataattagagatattacctcacgcctggccacatcctggtagcaagacacagacgagaaacagcaaaagtgaccagcctctatattgatgtatctagagtaattctagtgtaattatcaataaaagtcttcagtgttagcattccaaaatggtcacaaaaactatttttataaaataaagtaccacccattttcccacaaaggaaattggatatgggctttaaaaattcaattagtaaaagtaggttagaaaactctaaacaaatattgaactctagctgagaatatgtatgctgatgtagttggcttaagtgtactgattttggcaacttattctaaactgcatcaaaaaataagatactttaacagatgcatggacagatatgtgataaaacaaatataataaaatgttaattatataatttacatggtagatatatgggtgttcactgggagctgccttcaactttactgtatgtttgaaactttctcttttttttgagacggtctcattctgtcacagactgctgtaatatgatcacagctcatgcagcctagagctcctgggctcaagtgatcctcccacctcagcctcctgaatagccaggactacaggcatttaccaccatacccagccaatttttcaatttgttttgtggagatgaggtttccctttgttgcccaggctgggcttaaactcctgggctcgggagatcctcctgcctttgcctcccaaattgctaggattataggcataagctactacacctggactgtttgaaacttttcatgctcaaatgttgaagaagaaagtgggttacaagaatagatgcgtggctgggcacggtggctcacgcctgtaatcccagcactttgggaggccgaggcgggcggatcacaaggtcaggagatcgagaccatcctggctaacacggtgaaaccccgtctctactaaaaatacaaaaaaaaaactagccgggcgtggtggcgggcgcctgtagtcccagcttctccggaggctgcggcaggagaatggcatgaaccatggaggcagagctttccagtgagccaagatcatgccactgcactccagcctgggcgacagagcaagactccatctcaaaaaaaaaaaaaagaatacatgcatttcccagattttttgaaattaaattatgtaatggttgatagcaattaataaatactaaatggccaggcgtggtggctcacacctgtaatcccagcactttgggaggccgaagcgggcagatcacaaggtcaggagatcgagaccatcctggctaacacggtgaaaccccgtctctactaaaagtacaaaaaattagcctcgcgtggtggcgggcacctgtagtcccagctactagggaggctgaggcaggagaatcgcttgaacctgggaagcagagcttgcagtgagcggagatcgcaccactgtactccaacctgggtgacagagtgagattctgtctcaaagaaataaaaataaaaaagatactaaatgaatcttcctctccttcacgattacctgtctgtcagaattccatttcatttccttttctctcttcttccagctctcttcctcctcatttaccctcttaattgactataagatacactgaaatgtttctgatacttggtgttttgctctgttttgttagtttttatttctgcttaaccttggtggaatgctttagaatctctaatagaacaattagtttgcatcttaattatccaactcatccatttctctagcaggtgaatgacacttttttgccctagtgtcaattactaatcttttgccctccaaaatccacctctcagtcctgacttcactccaacacactcgactcctgaacctacttcctagagaacaccatgcctccagtcaaatccaatgtctctgcaactgaactcatattttctttcatatctgttcttcttttgttttttctttgagtaagagcgctacaatccctccagtttcccaaaccagtgttgttgcttactccttcctcttccccagtccctataatttaccagtcacgaagacttctctattcttttttcgtaatgtctcttaaatgcattcatgtgtcccaccccaagtgccacttctcttgcctccttcccacccccacctccaactctcatctgctccttcccattatcaccaatgcttctggactggggtgatgattttgctccccagggaacatatgacactgtctggagacatttttggttgtcacagtggggtagggggataatgttactggcatcaaggatgctgctcaacagcctcccccgaccaccgcgccccataagcaagaatgattcagcccataatatcagtaatcccgtggtgcagcacacttgctttagcatgaagactctcacagctgacagtcaataaatgtttactgaaggagtgaaataaataagaaattaaaattacatttctcagttaactgaattaaatcaggaacttaaagggagaaccagttaactgaattaaatcaggaacttaaagggagaaccagttaactgaattaaatcaggaacttaaagggagaacctaatggaacaaatcagattttaaatagtctatctacagtaccaaatatcttaaatagagaattaactcttctctaacgcactctaagttctaaaaactacacaatctatataaatatttcaattcccaacatacttggtaacattataaaaagtccttgctcataccagagatttcctggtcactagaatggaattcccagagctcattttgcagtgatgattatgtgtatgacaaacaattagttatatagctttccatttgtaccaccattattcattttatcaccaatgcagaacttaaagtgtttatacaacagagaaaaagtgaaatattcctctccccaagcaatacagaatgcactacaagtttgtttctacactggaaaaaatttaaaggtaaaaaattctaggtcacttaagttcctccctgctacaaatgaaacataaaagccattgtctcctaacctctggaaaactcactagcttcctctgaaaaattccctcttcattatataggcatactttcgcattcaacgcgtattagccaaaacgtttatggataattggcaaccgagaaacaaaagaagacataaaaatatttattttgttcttatgtcacagagaaattcctatgaagctaaagaattctggtattgaatttctagacatatataccctttctaagtgaagacataccatttctgccactgtgcccggctttgattctcacttttttctgtgcccaaatatttttcttatctattttgcttgccttaatccttctcacctccttttcatttatttttatttttattttttgagatggagtctcaccctgtcgcccaggctggagtgcagtggcgtgatcttggctcactgcaacctctgcctcccagattcaagcgattctcctgccccagcctcccgagtagctgggattacaggcacccgccaccacgcccggctaatttttgtatttttagtagagatgggattttaccacgttggtcagcctggtctcaaactcctgacctcctcctgacctcgtgagccgcctacctcggcctcccaaagtgctgggattacaggcgtgagccaccgcgccctgcctttcacctccatttttatcatgaatttcagcttctccaagaggcctcctttaaagtaggttatttccctgtcttagagcatctgtcttaaccttcaaattcttgatcacaattgtaattatatgtttgattattgacttgttcattgtctgtcttggctcttacccattacaatgcaagttccctgagggcgggaagcaagtgtgctttatcaccaccatcacctgctggcaaagcgcttagaactcaaatattctcaatgaatcacgaacaaaatcaggagtaaaacaaataggtcaggcttcaagcaatgcttttgaagccctctttctatttttttttttttgagacggagtctcgttctgtcacccagactggagtgcagtggcgccatcttggctcactgcaacctctgcctcccgggttcatgcaattctcctgcctcagcctcccgtgtagctgggattacaggcacacaccaccacgcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacaatttctcctccttctaattagcagattctttcttcaaatttagatcaactgttatccagaaaagtcttcagtaatccccttccatgctcttttggaacccttcatagtttctttgtgtaattaattattttatctaattagttatgcaatcatttgtttaaagttgaccttcctcactgacatataaacttgctgatggccagcatctaccttgcaaacttggtcacttgcacataaaaagcatcaataaatattttcaggatttctgaataaatacaatgctatgattctgtgatagattcattcatatcatagtcagaactggaaatgatttatcagtctataccatcaaactttttatgtaaagggccaaatagtaagtatcttaggctttatgagccacacgtaaggtctctgtcgcatatttttcactggtttaattttgttttgagcaactttgaacatgtaaaaaccattcttagctccaggctttgcaaaaccaggccaagtctatacttcagttacaataactacatttcaaatgtctaaatgttccattctaccaggatactagtcatgcagttgttatgcctctttgtatttctgtctataaaacaaaactaatctcaaaagctgcctttgatatttcgagtgacaaagcaggacacaagagatctctctattttctccatttcttctggtcattagagaacattctttggtctcccaggattttttggaggggaaataacaaatttttcccctacagaacttcagtttgcctgcatctaactggaactaaatatctaggaaatggcatccctcaattctcaagtgattaattatcataacagtagattgttgtattcatgttaccgaattactgaaaatagccctaagactaattctttcctaccgctcagataatgtatatgacaaaaaataaggtcagcaggatcttgcatgtgtgtgactgaaggtaaatgtgtacgtgcacatccagccaaaaattatcaaaggagttccagaacagtgtggaggagagaaaatgatgacctgctggaactctacttccatgaaagcatatgccccgaaatccttaatcaagggtctctgtaccacaaccctatcatcaccctgggcctggtatttccgtgtttagcggtagtgggggaaattatcctgtgcttttagcagcatccttggccattactactggatggcggtggcacagccacctcgcccccagctgtaacagcccaaaatgtctccagacattgccaagtgtctcctggggcagggggcaaaaataaccccaggctgagaaccccaggcctaacctctctctaaagaaacttcacctccttctttagtgataactaggctttcccttgaggatatcctcgccctgcaatcgtctcaggtggaggcacttttttttttttttttgagatggagtctcgccctgtcgcctaggctggagtgcaatggcatgatcttggctcactgcaacctccgcctcccaggttcaagcaattctcctgcctcagcctcctgagtagccgggattacaggcatgcgccaccacacccggctaattttttgtatctttagtagagaggagatttcaccatgttggccaggctggtctcgaactcctgacctcgtgatccgcccacctcagcctcccaaagtgctgggactccaggtgtgagccaccacgcccggccaggtggaggcattttaagtcaaatccttcacatctgaggcaaaaaacgtgggtaacatccttgttccccatgaacacttccagatgatttattctccttcaaaaccatgagcttttctgaaaattcctgccactggctagaccctactatccctcctcctttctgccatccactaacctctagttattccctcccttcgtcattcactaaataactcacctcctgcctcactgccccacgttccacctctgctcctatcattcttagtgatttggacatccacatggagagcaaaccattgctcaggaggatacctcgcccgagaccttgatccccttgcctccagctaacattttctccaccacaaaacagccacccattcccatctcacagcctcatattggtcactatcaggaaccgtacaacctccagaagatcaattccaaagtttactccaaactctccagtgggttcccatcaccctcagagtaaaatcaaatccctcactgtgcagtcctgataggtaagttagcaacaacgaggaaggggggtcccaggtgggggagaacaattgttctgagtgactgctaaccacaaacaacctgcttgcacactatcctgttcccagacacctcactctgcatgcagccccagcagcgtcattttatttgcacagtggccccacaagcaggaccctatcaaactttcccccagcccctgtctcttggcagacagccccttctctgctgtgctacccgttgccctttgcaacgtacttactctctaataaacttgcattcttcacctcactactgtttcagtaaattattttataacccaaggcaacagccccaatcagtcacaacccccgcaacactcaccagatgcaagagccacctccctacctcccacctctcaccactcttgcctactggccttctcctcctcaaacacactgagctcactctcaccatgaagacttttgcactagaacactcttcttctgcatacatcaatgactccctcactccttcatctcattcaggtctctggtcagtggtcactttcactgagagaacttcctaataccctttctgaaggagcacaccccattactcttgttcccattatccactttattcttcagagagcttagcccttgaatttatgtaagtctgtgatctgtctcctactcctggagcgtaaactccatttcagcagtccctgctgtgtgtttaccaccttctactcaggacctcaaggagtggctggcaaagacgggataatcaattaatttttcgatcatcaatgaatctatgtacaaatgcctgaatgcagcatctaactcctgagaactaccttctacttttaggctcttactctagaaccccttctactgctccatccagcatgctgaccttgaatcagttgtccctactactgttgcacaatccatcactcgtgctgtgtcacttccttccgcagcggcttaaattccatgttctatcactataattacatacttcaaacacttcagctttcttatccctctctccctccaccacgcttgtcaggtgaaaccacgacctgtttcggcgcagccatccactttctctttgcctgcacctgaacaactgaacattgtaaaagaaagccacacaggtgttgtctgtcttgactttcagtgcacagctatatacctcaaggaggccaacactgctgtgggttctcatgtaagcttccactccaatacaatgtttaaacaatcttctccccacctgacccgttcttcccaatgatgagatgatattgtcaaatgatttactgagacaatggaagaatctgagacaaattccaaatatgcaaaaatgctcacacttgtcttcctcacaatggaaagaggccctgtgacaaacaagagcaagttctttcccttgtgttctgagctgaattccttattgcgtgccaagatctcacttctttggctatcccttctctcctgaaacacaaacacctccctctttgctggacattcccagtatcacacaaacatgctttaaaaaaaaaaccgaaagaacaaatcctcccttgagccacatcactctccgctcccagctacgattccatttcttggcttcccttcacatcaaaactactaaccggaattaactacgtttgctacctctccctccttgctcagtctgctctaatttgatttccatgcccatgatgtgccagaagctgctgggcagcatggtatcaataactttttgtttccattttttttttttaaagataaggtctccctctgtcacccaggccggaatgcagtggccctatcataactcactgcagcctcagacttctggacaaaagcaatcctcctgcttcaacctcccaaagtgctgggactacatgggtgagccaccacacccagccaacatttctgtcttcatcctacttagtttatctttcagaaaaatttcactgttgatttactcctactccttaaagcattttactctcagctcatgatattcccttcccctattttctctaactcgctagccattttctcccaagtcccttttaatggcttctctacccaacacggcaatgtcggagtgcttcctggctcaccccacagcttattctctcaactctttctatattctctcccataatctcgcccagtctctaaataccccattctttgaaaccgggcagacctttgtgaacgcactaacagagtatggaagaaggggccccatacctggacggaatgcatggggcagttggggcagctacgtgaagcctctgaagacagagcggagctccacaagactgctttcctgagatacaggtcacaaagacccactgataacacagaatgtgaaaaagaagctagccaaaacccaccaaaaccaagatggtgatgaaagcgacctctggttgtcctcactgctcattatatgccaaagataatcattagcatactaaaggaatcgttagcatggacccgccagcgccaggacagtttacaaatacaatggcaacctcaggaagttgccctgtatgttctttaagggggaggggccctcaattctgggaatctcctgtcattttccaaaaaaactcacgagtaatccaccccttgtttagcatatgatcaannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagatagaggaattgcaatagagaaagagtcttacacatgagatggctcaagagagactggaattttattattatgcaaatcagcttccctcaaaattcagaggctagggcttttcaaggatagttcaacaggcaagggaatgtgtgctactcattggttggggatacaatcacagaggtgcaaaaatggtcctcaggcctgctgaatctgcttctaggtagggacacaggactggttggtggtatgggtggagtcattggtcatcagaaatgcaaaaacctgaagacatctcaaaaggccaaccttaggttctgcaatagtagaataattggggaatctacaagtcttgtgacctccaggataatggcaagaatcatttctgtctacactttagcagaattcaggctcctctcatcctcctaacctggtggtctttcattagttctacaaaggtagtttagttttggagaagggctattatcatttaaactgtaaactaaatttcccgaaagttagcatggaccaagcctgggactaattaaagacagtttggagattaaacacaagatggggattggtcagatctctttcattgccataattttctcactgttgtattttttgcaaaggtgatttcactttaacgaagcctacctattatctattctctagaagttcattgttaagtaagtagttgaggttctgattccttagagtgactcagtttcaattcaatccttgtcgtcatttgaatatctcaaattgacaaaccactttgtgtatgattttatatagatgtctgattagctgttctgcaaattcacaattgcagtcaagattctcttgtccagcacaggaaaacaagacaagcagcaccttttccagtagtctctcaataagactttttcctgtgtttatagatgtaaaatttgctaatgagcaaggcctgagagctaacattgaagttgttctgaagataacctattcagtggattagaaatatatattcatggccgggtgctcctgtaatcccagcactttgggaggccaaggcaggcggatcacctgaggtcaggactgcgagaccagcctgggcaacatggtgaaacgccgtctctactaaacacagaaaaattagccagatgtgttggcacaggcctgtaatcccagctactcggggggctgagacaggacaatggcttgaacccaggaggcagaggttgccgtgagccaagatcaagccactgcactccagcctgggcaacagagtgagactctgtctcaaaaaaaaaaaaagaaagaaagaaagaataaagaaatatatactcattttagaatagaatattcattttttaaaacactgcgttcatgttctaatgatgctgtaacaaattaccacatacttagtggtttcaaataacacaactgcattatcacacagatctggaggtcagaagtcttaaaatccaaatgtcagcagggctgtgctccttctagaggctcttagggaaaatctgcttccctgccttttccagcctccaggggctttatgcattccttggctcacagctctttcctcatatcactctgacctcggcttttgcctccccgtttttcttactccaactctccttcctccctctttcacatttaaaagacctttgtgattccactgggcctacctggataatccaggacactctccccatctcaagatcagctaattagcaaccttaatttcatctgcaaccttcattccccactgccatgtaacatgtttatttacaggttctagggattctgacatagacatctttaggggccatcattccgcctacaattgtgggccgatgtcttccttctctcccgacctcagcaggttgggtctgggtggacacttgaaattggtagaacagtccacagttgggatgagtcaaacatgactgagtacttgactgtgaagttaggtagaactcagccttgctcagctatcgcaggccacgtacatgctggttagtgaacagaaggaaacaggaggttgcagatacagggagacaagtagcagcaagagacagaccagagatagagaaagtagttctgatgattttctttttttgagacagagtctctgtcgcgcaggctagagtgcagtggcacgatctcatctcactgcagcctccagctccctggttcaaggaattcttgtggctcagcctcccaagtaattgggattacaggtgcacattatcacacctggctaatttttgtatttttagtagagatgagatttcaccatgttggccaggctggtctcagactcctgacctcaagtgatctacctgcctcggcctcccaaaatgctgggattataggcgttaatcccagcactttgaattggaaaatggattttctaaactgaattagaaaatagtaaaaactgggttcggatttagtttcccactccgctattttctttccagtatatatttcagaccagtaaagaagagaggtaaataaccaattaagaaaagtttccttgctggatactgagatgtttgatggttcctgccactcctactggcacttagtacatttgtggtctattatgcagcttagcaattttgaaaaacatttttattctctttagctcatcccttcttaaaaataaatattaaggttttggggagctgaggatacgaaattcgtaatgtatacaacagctgaccagttcctgtcaataaaatatgacaattaaattcagaagcgaggccctttccttgcttatcttgtttagtgagacaagctcattcctaagcagtcccggccaatatgactttgaaatagtcatccctggaatccatttcgggtttctttttggacccaagagctgagctgaatttggacatcaggccaaggctgaacagcaacagggttcaggccatttctcattaggaaattactgcctctaaaccaggcccaaagtgagcactattgattttttaaatactttttttattaacaattttggggaggtataatttacatatcataacttcacctgttctaattcagttgttactggaaatggggtcttggaactcatgcaagaaataatttggggtgagtccacagagtaaaccgaaagcagatttattgagaaagtaaaggaataaaagaatggctactccataggcagagcagcctcaagggctgctggttatccatttttgtgattatttctcaatcatatgctaaacaagtggcagatattcatgagttttctgagaaaggggatttcccagaactgagggttcctcttccttttagaccacatagggtagcttccagacattgccgtggcatttgtaaattgtcatggtgctggcaggaatgtctcttagcatgctaatgcattataattagtgtaaaacgagcagtgaggatgaccagagaccactgttttttggcatcttggttttggtaggtttgggctggcttctttactgcatcctgttttattagtgggcttcgtgacctgtgtcttatgctacatttctatctaatcctgtgaccaagaatgccttccctcctgtgaatgcagcccggcaggtctcagcctcactttacccagcccctgttaaagatggagtcactctggttctaacacctctgacataaccatcaccacaatctcattttagaatatttccattcccgccacatgaaagtcatgccccattcctgcgcccagccctaggcaacgtgagtttactttctgtctctacagatttgcctttcctggtcatttaatataaatgaaatcatgtaagatatggtatttttttcctacttcttaacttaccataatgtctttgaggtttatttatgtggcagcatgtatcagtacttttttttaaatcaataaagagtattctgttgcatggctagtccacattttgcttatccattcactaaaactattgattttttaccactagatcatggtcagaaaatgtagtctgaggaacctgccagatgtagtctggtcaggggtatcaggtgtcttaatctgttttgtgttgctataaaggaatacctgaggctcagtaatttatttatttttattttatttatttatttattttgagacggactttcgctcttgttgcccaggctggagtgcaatggcccaatctcggctcaccgcaacctccacctcccaggtacaagtgattttcccgtctcagcctcccatgtagctcagattataggcatgtgccatcacacctggctaattttttttgtatttagtacagacggggtttcaccatattagtcaggctggtcgagaattcctgacctcaagtgatccactggccttggcctcccaaagtgctgggattacaggcgtgcaccactgcacccagccaaggcttggtaatttataaagagaaggggtttatttggctcactgttctgtaggctatacaaaaagcatgctgctggcatctgcttctggtgaggtctataggcctcttccattcatggttagaagtcctaggcacactggcttgggctcaatgtttcctagcccacatcttgaaggctgtggtggatgaaatcagcatgtagaggcaggaaaaccccggatcaatcagaaaaacacttactccttggcactcatatgaggatggcataagtagagggtcttggtagaaaagatgacagttcagtacacttagcaacaaagcttgagaatgtctagcttctcacacctggcaagcttcctcagtcttgatgaattaacgattctgcctttctgtctttccaggtgctttggctatagaggccttgagatatttcggttacataactggctcttatttccgttgtttaacatttattgagcatttaatatgtgtgaggtcatttgcatacattgtctaattaaatccttgcaatgatcttataaagtagctgcctgatatggtttggctgtgttgccactcaaatatcatcttaaattcccacatgttgttggggggatccagtgggaggtaatttaatcatggaggcaggtttttcccatactgttcttgtgtgatagtgaataagtctcacaagatctgagggtttcagaggggagtttcccgcacaaattctcttctcttgtctgatgccatgtgagatgtgcctttcaccttccaccatgattgtgaggcctccccagccatgtggaatgtaagtccattaaacttctttcttttgtaaattgcccagtctcaggtatatctttatcagcagcatgaaaacagattaatacagtaaattggtaccagtagagtggggcactgccgaaaagatacctgaaaatgtggaagtgactttggaactgggtgacaggcagagtttaaaacagtttggaggccaggcatggtggctcatgcctgtaatcccagcactttgggaggctgaggcaggcagatcacctgagatcaggagttcgagaccagccggaccaacatggagaaaccctgtctctactaaaaatacaaaaattagctgggtgtggtggtgcatacctgtaatcccagatactcaggaggctgtggcagaagaattgcttgaacccggaaagcagagattgcggtgagctgagatcgcaccattgcactccagcctgggcaataagagtgaaactgtatctcaaaaagaaaagaaaaaaaaaacagtttggagggctcagaagaagacagaaaaatgtgggaaagtttggaactccctagaaacttgttgaatggctttgcttaaaatgctgataatgatatggacaatgaaatcaaggctgaggtggtctcaggtggagatgaggaacttgttagcaaccgcagcaaaggtgacttttactctgttttagcaaagagactggtggcattttgccttgctctagagatttgtggaactttgaacttgagagagatggtttagggtatctggcagaagaaatttctaagcagcaaagcattcaagaggcgacttgggtgctgttaaaggcattcagttttaaaaggaaagcagagcataaaagtttggaaaatttacagcctgacaatgaggtagaaaagaaaatcccattttctggggagaattcaagcaggctgcagaaatttgcataagtaacaagaagtcaaatgttatccccaagacaatggtgaaaatgtttccagggtgtttcagaggtcttcatagcagcccctcccatcacaggcctggaggccttggaaaggaaaaatggtttcatgggtcaggccccaggtccccgtgctgtgtgcagcctagggacttggtgccctgctccagccacggctgaaaggggccaatgcagaggtcgagccatggtttcagagggtgcaagccccaagccttggcagcttccacgtggtgttgagcctgcgagtgcaccgaagtcaagaattggggtttgagaacgtctgcctagatttcagaggatgtatggaaatgcctggatgaccaggcagaagtttgctgcaggggcgaggccctcatggaaaacctctgctagggtagtgtggannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaattcaaggctgggctaggtggctcacacctataatcccagcactttgggaggccaaagcaggtggatcacctgaggtcaggagttcaagaccagggtggccaacatggtaaaacctcgtcactactaaaaatacaaaaattagctcggcgtcgtggtgcctgcctataatcccagctacttaggaggctgaggcaggtgaatcccttgaacccaagaggcggaggttgcagtaagccaagatcgcgccactgcactccatcctgggcagcataatgagattctgtctcaaaaaagaaaaaaaaatgaattcaaatgcacataataatttctgtttgtccacttaagtcttctttttgtctacggtaacaatgagtagagcatatttcattagttttccgggactggctgctgttggttttgattaacagcttcatctttcaacaggttcaagatcttatttcttgggtttctttttaacctgaattgttatgtcttccttgtctttaattttagcttcgctcacatattcatccaaaccgatgtccatttacatacatatcaaggagctcttgcccatctggtgagcaagggatcaccacatgtttagcaaaatcagcagatctgccaaacccaaacaagtccagcaaccaaaaacatgcatccaaaaatatcctgctaggcagacatttaaagataagctcatgaaggattaagtagatttagaatcacctacccctgcaaaaatggttgggtcaagtaaacttgcttaactcttggtgatgtaagtagatcatataatatatgccacagttaggattaaagaaatcctagaagagcttgttaatgaataatcaacaattatgtaagataagtgtggttggctggaaaatatgcatatgacccaaaaaaagggatctcatttggaaaaaggcatgagagaaatgactagagtaggttccaaatacacaaagaagcctagcttcagaccactgaaatataccatgatctacaagtcaacaatataatctgccaagtagcaagcattagtatgatccctgttttctcttgctagcagttctatccttatatcctcctcccaaaatgcagctgtcttctctacctcagtgaatggcacaaccatctacccagctgcttcaaaccaagaagtcatccttgatttccttgtgttctcttgtcgccatgtctgccccaccagaagtcccatcagccatacctccagaatccaccccaacctactaactggtcttcatctcccctgtcaccgtcctgccccaacctacagccaccatctagaactaaactactgccatatcccacctgccttcctgcttctacttctgtgcctgcctacactcaatcattcaaagagcaggctgggggccgggcatggtggcttatgtctataatcccagcattttgggaggccaaggcaggaggactgcttgaccccaggagttcaagaccaacttgggacatcacagtgagaccctgtctataacaacaacttaaaaaattagctgggcatggtggctcatgcctatagttccagctacttggaagggtgaggtggaaggatcgcttgagcccaggagatcgagactgcggtgagccatgattgtgctactgctctccaacctgggcaacagagcaagaccctgtctcaaaaacaaccaccaccacaacaagcaaaccaagaccaggccaggtgatccacatgaaatataagacaaatatgttcttcccctgcataaaacctcccaaaagctcccatttaaaaaaaaaaaaaagccaggtatggtggctcatgcctgtaatcctagcactttgagaggccgacgtggcagatcactggaggtcaggagttcaagaccagcctggccaacatggcgaaaccctgtctctattaaaaatacaaaaattagctagaaattgcttgaacccaggaggcggaggatgcagtgagccaagatcatgcctctgatcttgctccagcctaggcaatagagcgagactccgtttcaaaaaaaaaaaaaaaagccatattccttccacggctttccagaccctgcctgtgctgtggcctgagcccttgctgtcctcatctcctgaagccccagcttcctctgagtcatgtctgtgctcctccaaggccctttggacttgccctgggagctgcctggaatggccttcccacagacctcatctcacaggctcccctctgcgctcatcctacccccacgccattcactccatcctgttggtcctgtgttccttcatatcacttattagaatctgagagcaattaattagcctatgcattctgaaaaaaaatctgtctctctccattttgaataggaaacacatttcaacagaacacattctcttgctcactaacacatctccagagcctaaaacactgcctggcttctagaatgaatatatttggtaaaaatctgtgaatgaattaacagcatccctcaaagccatctttgtcttttaaagttcttgacatcttaatcatattaaaatgtaagagaaaatgtgaagtcttttcatactttctataacactcgataaagcaaaatgttaggccctaaaattgtataaaataacataaaacaaaatcagaagggttgagtcaagtaggctaatacataaactacatcagtgaagagccccaaactgaagatctttattgctgtaattgcatacataagtgcaaagatgagaacggagaggataaggaggtgggggagaggaacccatcagggaagggaacaaagtgggggggggtaggtaggaagggtaccagttatgacattattcccacagcatgttctgtctcgaagggcaactgacctcaagaggattgttagttagaaatgtccccaacagccattttctggaaatcagcagagatactaattatcgaaaatttctctccccatagataagcacattcttcctcatccctcattgacaccccagtcctcagaaccccctgtatcattatatcgtcgccctctccagttttcaagaaactccttgactgcgcatccaatgaacgcatagttctgttactgagctgctatttaagtatagggcataacatttcatacaagagtatctttaattttcttctttgttttatgttcactttgatgggggagtagagaggtcgtacagagccaatgaataaaggaagacaaaatgcaatcacatcaagttgaaggaaagaaattcccaattctgaggaaatgccttcacacaacattaagggattttatacctttgttgtttgtttttcacgctcgatgccacacccagctgccctccaaaataagatgatggggaagagttactagtctctaacacaaggatggagtgaggtgagtataagaaacaaccaggttagttgattttaaaaaagagagagagaaagaaataggattaccctattcttttctgtttatccaattctacatttatcgactggcaagactgttcacttctgggctttaaaggaggaaaatgccgttaacttgggaacagtttgaatcaaaaagcaagtgctagacatggctagtgtttgcctttgcctgcactatactaattatagttccaacttgccaaatcaatatatcttctagaactgagctctataacatttaacacaccatcattctagcgttctgcctctacaataacacagacctgtcatacaagagaagggaggaaaacatttaagttatatattttttcccataagagaaagagatctacctagcagcgattagctgcaatgtcattttaaaaaattgcagcaggaaaaatgtaaacaaatctcaggaaggaaacttcacaagtcacagcacaacatggggaattttctttcaaatgggaagcatcaagaatcaagaagctctctgaatattttatgtgatttattttcttaatactaaaggaagattgaggaagactgcaggctgtaatgggtaaagaagaggaagcaaagtgaacaggcagatatagacactttattatttattatcgtatcttgacctcagctggggctttgtcattgtctttaaagctcatcattattttattcctatatattgaagaaggctgataagagagcactgttggcagcaacaaaacaaagtaactaacccatatgatgaaaaaccacggccaggcgcagtggctcacgcctgtaatcccagcactttgggaggccggggcgggtggatcacgaggtcaggagttcaagaacagcctgaacaacatggtgaaaccccgtctctactaaaaatacaaaaattagccaggcatagtggtgcgcacctgtaatcccagttactcaagaggctgaggcaggagaatcgcttgaactcgggaggtggaggttgcagtgtgtcaagatagtgccactgcactccagcctggggaacagagtgagactacgtctcaaaaaaaaaagaaaaatcattatcctcaactaaaaatcctgggctctgcacaagtgagccaaccttcaattttgttatgagaatttcatccacgagaattcttacacatctgggttttagaggcgtactcatcccaaccaatgccacacacataccacatacctgagaacgactggcaatactctgccttgacaatgagaatccagctgtgttcatattcattatgaattgtgaatatgcctggtgaattactgttgtacannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncctcaattttttttaaaaaaaagggcttgcatatttgttttactatatttattaaattcacatcaaaaataatattcattaaatgagagtcggggacttattaatcattaatgttctccaattattgctgtactaatcccctcacgctcttcccacatattcttccacactgtagctaatcaaaagtgaagcttgtagaatcagctttgcagtcatacactagctataaatactcctcttaccttgagaagcaaaactcacaagcctagataaacatggacaccgcagttagggggtaagtttttattcaccatcagaacaaatgttgcaatcctcttgcacaatgaagaagtaaaatcaactctccttgcactagggttttattgggcctgtgaccattgacactataaaaagtaaaaagtattttatttgaacagggctatacaatgtaaaaggcatttctgcatctactgtcagctctgtgaagtaggcagagcaggatttatcatcacaacttcaaagaaactgaggaatgaaaaggttctagagtctgcccgaagtcataagattagaaagtggcagtgccaaaactggaggcacactctgctctcctcatttttattccagggatcttctacttctgactcagccagcaactctgggaaaagcttctgaccaactttctgcattcggatataaaagaaacagaccatgcggtctgtgccctgcaggggctaacaacctacttacaacaaccaaagcccaagaaggaacattttaaaagcagtctgtgtagaacacactgccttaattaaacatccttagtggaactgggaggttggggaggagcaaagataagatataaccaagcacttaggagtccgtcatatcccaaagaatgtcctaggcctactagaggaagacaagctgaagaaaacatgctccctgcccttaactaataataataatagtaataataatacaataagatgatgtagctggcatactcttgtagaagagatgcagagtacaggctggttgctaactgctgacaacataccgcagggagagtcagtgtctcaggatgatgtatttgctataattgatgtggaacacatcttttaatacccttctcaatatttctagtgtcaagtaatacgaatgcatcaaatgtgcgtctcagttcaccacagagaacagccaacttcttcaaaacaatattaaagacacactgtaggcactttttcttgaagactgagaggatcttcctgcaagagtatctaaatgaagggtgtacataggttcagtgagttcaacatagagcagggagagtctcccattttactgagacaagaaagtgtccaaacaggaatagaagctttgatgacagacagtaggagatgctcttctcccaatacccttgatggtgcatgtaacctttctggttttactcattccaaagactaatcattgtcatgaggacgatcaagtgactcagtgacaatccacttcagtatgttcatcttcagtatgttagagccagaagaaatcttagagatcatttcgttcaaaccactaattccactgtaaagaaatcaaggcccaaatacttgagtcatatgtctaaggttatatacatgagagggctaagtgtaagttttgtactgagttttatgtcctatgatagcattcaattttaatcatctgtttgaaacttatattttaatagcctacaaatatgtcactgatattttttccttgtgtgcatgtgggttttttgggttttttggggtgtttttgagacagagtctcgctctgttgcccaggctggagtgcaatggcaggatctcggctcactgcaacctccatctcctgggttcaagtgattctcctgcctcagcctcctgagtagctgggattacaggctctcgccaccacgccaggctaatttttgtattggtagtagagacagggtttcaccatgttggccaggctgatcttgaactcctgacctcagatgatccatctgcctcggcctcccaaagtgctgggattacaggtgtaagtcactgcaccaggccccttctgtgcattttttaatggtagaaggatgaacaagacagttgctgtattttaaaaggtgtcaacacaggtttctgtttcttccaggccactatatcttatgcaaaatgagaattaaaacagatgtcttgtcacgttaaatttttctctgaccttatagctcagacataactatctaataaacttccctgatatcccatgttatctatcaattgaaatattaggaggaaataaggatgttgacaagcaaatctattataagacattaatgataattgcttaagtttatgatgtcactgaatacagagatatcacatattctgtatctcacatactaacggcttatacttgccaaataagaaaaaaaataaggagggccatttatgatcaaatagaaattaccatgtctagttttaggtccaaaatgctgcaggttttatagtaaaaagaagactgctacaaggataaatatctagggttgtctcaaagaaagaaaacaaacgtgaaatccaatagctagacaaaccattaaggaaaacaagtcacaaaacacacaaggatgatgaactccctgccctcatctgcaaagatgaccatggctaataaagggatttggaccaacataaagcagagtgaaactctactacccctggaggaatcaaaattcactgaatccaggtacacctttgatttagacactcttacaggaagatcctcttagtcttcaagaacaagtgcctacaatgtgtcttcataattgttttgaagaagttgactgttctctgtggtgaactgaggtgcacgtttgatgcatttatgttacttgacactagggatattgagaagggtattaaaagatgtgttccacatcaattatagcaaaaacatcatactgagagtattcaaaaagtgactttgttatagagagttctcggtactacatgtctattaaaatttatataaagttcaatttgactagcatcaagggattttttttcctggggaagagagaaaaagacactatccaacttcaatttaccaaaggctgaaaatgacaaaaataaatagagaaatttaatctaataaatctaatgaagttgaaaaaagaatatcatagaacacatatgatgataactaccaaccacttttcaggacctatgacaggttatgaatttcctggtatttttttaaaaggtcatgaaataaatcaagattaaaagggaaaaactatagaaagacaaaagaaaaatgtttttttaatttaattgttgtatctattaagttaaatccataaaagtgactctaataaatggcagcttctaaaatcacttggttgaagagttttaaataaatatttaaagcagtctcaaataaaaggaatgagtcacacagaccacagctgctgctcttacttttcaagtcatacatcattgactctccgagctggaaagaatatcagtggtcaaattcagcctctccagggctacgaccaccccatttgatcttcactcagtctctattttaacatctccagtgatgatgaaattgctcttctcaagaccacgcatgccaacactggacattctgtaagggatggcttcccttatgctgacttgaatcatacaaactgctatcacatctagctccttcatcctgaacacattcagtcttctgcttgaatataaatctgaggtttcaattgtgttcctacagtcaatggaaagctactggaaaatttggagagtggcattaaaaatcctcctggaaaaggccgggcatggtggctcatgcctgtaaaaccggcactttgggaggctgaggcgggcagatcaccttaggtcaggagttagagaccagcctggccaacatggtgaaacctcgtctctactaaaaacacaaaaattagctgggcgtggtggtgggcgcctgaaatcctagctacttggaagggctgaggcaggagaattgcttgaacccaggaggcggaggttgcagtgagcaagatcgcgccattgcactccagcctagcaacaagagcaaaaactctgtctcaaaaaaaaagaaaaaaaaatcctcctggaaaggaggggatgagtatttaaaaacatatttttggaggtatattttaacaggatgtagagactaattaaatataaggaggaggaagagtggcatagcttggctaacttgcacttccaaatttctggcttaagaaactgggtaaagagctttgttagatggtattatctaccaggaatccaataatacataagggagcaggttgggtttggagaaaagatgagttcagtttgaaactattcccgtatctatataaagatgtttaaaagacaatatgaaattggcacctgaaattcacaagagagatctgagctagaaatatgatttatcaacatgcagggaaatgttggatgtttcatagactactcagggtggatgttaacacagactacatatgaagcctggggagaaagcatcatttaattaggaagagttataggtggaagaagatcatatgaaggtgtctaaaaggcatcccaaaaggcagaaggcaatctaagaaaaagtatgcaatggatactcggggatgaatggtgttctgattggataaaatactatagagttgtttaaattatgcttttgaagtattttaaataattagtgagatttttattaatgttcaatgtaagacacaacgtactaaattaaaacgttaataatgtttctgggttttaggaatatggctccccaaaaagtgtatggaaatccacaaaaacttaccatatttaatgctaaaaggaaagccacaataattccaaaagaatctacacaatgtatactgttctatgattatatttcaattatttgctgtaaaaaaaagcactacctttaaaatctcatataactgtaagctatagattttactaaataaactttacatttaaataacgttcataaaggaaatcatacagtatttatacctgatagatactgaaaacatttctttaccttaacaataatcaactagaaataaaactaaagtaagatcccattcccattagtaataaaatatatgaagcatcaggaaatagttcagcaaaggaaacacaaactctttatggagaaaagtctaaactctattaaaatgaataaaagatgatctgaataaatgaagagatctttcacattctcagataaaatgatattacaaaagtggaaacagtcctcaatttaatctatgaattgactaaaatctcaatcaaacaacttcaggattttttaagaaactcaacaacctattctaattttattctaaatttatatggaggccgggtgcagtggctcacacctgtaactcccacactctgggaggccgaggtgagtggatcacttgaggccaggagttcaagaccagcctggccaatatggtgaaacccaatctctaccaaagatacaaaaattagccgggcatggtggtgggcgcctgtaatcccagctactcaggaggctgaggtaggaggatcacttgaacccaggaggttgaggctgtagtcagccgagatcacgccacggcactccagcctgggcgacagagtgagactcggtctcaaaaaaaaaaaaaaaaaaaaaaaaaagaagcataaactaggaaagcattctggaaactaatctgacagtacctactcaaataaaaccttttatttaatgacctgacaattccacttccaaatgtatatcccagagaagttctcccatagctccctgtatggaaatattcatcagactgttttctctggtggccgagaagtacaggaatctagatgtttatcaccatgctcatgtttaaataaaatctagggaatgtgcccagagacaccacattgaacagctctaggggaacattactcacacacaataaaaggtgaatggtgaccctggtgttgaacaacccagcaggactttcattcttaggaatagtatataacagtgggaagcaaaaacaaggtggaaattcacccagaaaagttcgtagatttttttccaaaggtggttgagttttgttttgttctttttaagaaacagaatgaggtctgcaactcagagtatttgtataaaccaaacataaacagaaatactatatactttagaaaaatgcagactaattcaaagagagcaatagataaaagcannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaattcagtccaatcaagacctttattttgctccttggtccaccgccttggaccgactcccactcctgctgcccaggcttttgccagcatggacctgcaaggtcccataagctttcagcgtatagtcgtctcctccagggcagtcctggcaacactctatcctggctattttaacatctatctttagatctgtcttttcttacgggttcccaactgtacagtgaagcaagatcaagcagttccttcattgcacagatgacctccaccctaagtgaagagtgaatctgaggggacaaacggaagttatctgtacatcaagctaacaatggatttttaaacagaaatcagaacaatcttttagaggtaatggatacatgtacacttctttaatggcagtgatggttttgtggaatatatttcaagactcatggaattgtacattttaaattgattggttctttgtgaaattgattatacttcaaaaacccttaaagaaatttaaatactaacacacctgggaatttgagcactttatactgatcaacataaaatataaataaaccattcatattgcaaaacgtattatggagtcataatgaggaattctggaaggctagaataattatatgtaacggtgtattatgattcacaccgctcatgtgtatgaatacatgttacatacgattatatgtaacagtgtattacgaagctagagtttaatttattgaagccatttctacttttatttttatttctaaagaatataaaacatttttaaactccaaatttaatatgttatctaagtgaaacaaagtcaaatgcaaaaatgtcaatatctgccttgtgaaaggcagaaggtccttggaagtcaatgataagacataaatgaaaggccaccagagggcagtgcaggactgcttaggacaatgagtgccccacctcaacatttccacaagctagcattgaaaacacgttttatcatcttacagaatgtctaaataaatatttatttgctattattctttcatataatttgaaaaataataaaaatttcctacaggaaacaaatagaaggttatccactaactggaaagtatatctttttacacattcagatatgtaaaattatgcaagaaagaatgagtcaagttggactttaatccatcaagatgtaaaagtttaactgcaaacaaatgaaacaactaaattaataagtaaaactcaaagtcttagaaggaaatataggagcaaatctttatgaccttgaatttggcaagagcttcttaaacatgtcactgaaaacgctagcagtaaaaaaaaataaaacagatgaattgtatgtcaacaaaattaaaaacttttgagcatcaaaacacactcttaagggagtataaagacaactcaagactgtcaggaaatattggtaagtgaaaaatctgataaagattttgtacccagaaaatataatgaacttttacagttcaaggacagaaacgcaaccaatccaactactaacgggcaaaggacttgcatagacatttctccagagaaaatattcaatcggtgaataagcacatgaaagatgatgagcaaccttagatatcagggaaatgaacatcaaaccagcaatgagataccactccacactcaccaggatggttataacaaaacagcagcaggttctgttgttgtctcaagacctggagctggaactggcttgggctctggagctggagttggggccagagcctgcagtggctgcatggctggatctggtgctcttgctgggactgccttagccctggagctggagctgttgatggagcttgcaaaggagctcgggagctggtgctacggtggttgctgctcttgcttccagctctgcagatttccctagagcggctggttaaggctgcagctggagctggtggtggacctagccctgacacacactctgaaggtggcttttgagcttgaactggagctggctctggagctgccagaggaggaaagatcactaacatgactgactttccatgtgccttctactgacaaaaatcttccctctgcctttctgagaaactcagagaaactccacacccaggaagtcttccagactgcagtcggccacatgattgggctgagtttctcattgctgcgtccgctcctcagcttctggcagctctgctctgtgcagtccagccctgtcccctccccacgggctcacatgaacccacccccaggcctcctcacacaccggctctgcttcagtgggccctgggtgctccaactgacccctgggtggctcctccctgtcgccagttgctccagcaaagcacagtggacccagcctccctggtggtgggtgctagcactgcacccaagggaaaaaaatcaccaaccaacacacctttgtttgaatgacttcaaggcatcagctctccctggtttcagggaacaaaggagcaggagctgtaggatccagggcattgagagggaccccgcaaactttccttgtccccctattgccagcaatagctctggggtccagttcactcatgaggccacttcaccacagccagggcagtgactatgggttcatttccagtgaggactgaaggctctaacagatggtctttgccttattcaagatgagtagttcagtgctctgccatgttcttccttaccccacatctcagtcctccagtctctctctgattcactctgctgcaaaagactctgaggaagtagaaagcattttccaaggaccacggagttcagtaagtcatctacagggaacctggagccatagacactttctaaagtgtcaccttggggtgaccaacccatagcatcacaatatgtaaggaggtaaactgaggccctcttgcctgtagccctcctcaccctaggaggtcttgtctcagttgaaggtgcccagtatggtggacatagaaccccaagtaccatctgcttctgactgctgtgagatggtctcagaccaggcagacccccgggtaccttacagctcccccaggccagcatcctggcatctctttccttcccaggccatggtcctggtcatggtgctagaagagtgcagagatgcctccagccttttcttacttttcccttgttacctccccagggtccacttttcctcttacttcagcgctgtgatcatcaagcatgtgggtgcatacccatggggttgctaagaggtacagggcccacataggaaggctagggaggggtccccagtctcttagggaaccccatgtgagatggttgagccctggcctaggaggtgggaatccattattttctggacatgcccacgtcctataccctcaccagtctctgcttgtctctgggactcaggttcccaaatgtccaatggcgggtggactggaactgtgtagataggagcttagcatggacagtgttattcccttcaccctgacagggtgaggcagagaggggcttcagggcccacacagactcttttcctgggaaggacacaggtggaccaagcgcagggccactgggcctcatgttcatggtgggcacgcccactgggccagtgtgggctgcagacagtgcatccctgaagctcccctgcacagcacatgggaaagtcaccagtggttcctccaatatctgatctcacgtagactctgtctcctgtagccaggaagtgtgtcagcctcacttaggtacatgggtttgagccggggggtagtttacctaagagaaactgaggcatggggacaattagaagtagacatggacatgggccaagtgaaagccccagatctttagaatcgtacagagcatccacccttttctctctcatgcagacaggactaagttccagagaaaggaagcaacccagaatgtgctattatttgccaattaattgaaggcaggaagtctgctcttcttctcagtccacgcaccagcagacttcagagggcctttgtccatcgccagttgggctgaactgtgatggtatctgagcttcaattttccttttggtcagttatacatacaaatctgataaaccttagtgccgtggcctcttcacagaaacctctccgtcagaaaaatctccctttttaacacctgagcaggacgaatgcagcagctttttaaatgagagctctctagggacctgcttcagccatgggccagggtcgtgcaggggccaggatccaggaacgctaactgcgcgtgcgctagcctggaagctgccgcgagccgcccccccgcccccgcccgcgtccttgtgccaggtgtctagccccagggactcagcagtgtgggctttagagcggctctcaggacttccaggaaaggctggggacgtatacatttacgccagtcaaaaagacccccttctcctctgttctctgaagctcagaatggtggagagtggacaggacttgaatgggcgaccccctgatactgcaagtttcctctggcttttgccttttaacccattaccgtccaccctggcacaaggcaggttgttgccttcagctgccagatatatggcatggcacagatgggcagtggaattccagctctgagcactcccggcccccattcttatcccagtaatggctggggcagcagaaccctgacacttcacccgcgagcctggataaaaaggctcccatgacctctctctttagccaaccccgctgggtgcaatattcagaagtgctccccgaggtttccaattacccgctgtgccccacctgtggcagagtaaacctacttcctccagactcgcatggcagggactgaccaagaaggaaagcggccctcactccagggttccgtggtgatacctgcatcagcacaccagcccgggggtctgcactcagcggggtctcaggccatcccgcagctcatctgcagtgctcgaaggaattgtgggtgagaaccaccagtacagggagcctcatgcagaaagagggacacaaacactacctgtgcatatatgcacatatatagtatagttacatttatatatgtatatacatacacacacacatatataaacatgttatgtatgtgaagaagcacattgatcgtctgcatggaggatctcatttaaaagataaataacatatcatgtcaacatgcatgcacacatttatgtaaatgaatgtgaatgtgagacagtgagacagactggcagagaaagagacaatgagagagacagaggctgagagatgtggccaaaccatttaatatggtcagatttggtggtaggacattgaaaatattttattttcttctctaagtgatacttaaatattttataatgttcacattatttttataattataaacagatgaacatcatatacatatgtatatagtaaacaaaactccacagaaacacacacattcacacacagagatattctgccaagggcccagtctgccctctctgcatgagctggcagcaagggaataagaaaatgccacaggatcctatcattgccacctccaggagcccctcggctgacagccggccccctgcaccgcccttgtttgtaggcacagctcaaatgccatccccctggagtggaggagatgacctcttctcctgaacacattactgagctgtcatttgtctccaccagactggcaggtttatatgtcgggtgacaaccagcagtggtgagaatgtgggaagcagaggccccggcagatcccctgggagaattgtttatggatatgcctctcttgagctggcagcatctatcaaagctctaaaccactgtctctttacctagcagtgataaccctgggacttcatccggtagacactggtgcaccatgtttcaagatttgtttttaagactattcatcacagcactggttgaaataacaaggaattggaaacatcattaacatccacatagagaggatgggtgaagaaaattgggtccgtgtgaaccagggaataccaagcagctgctgaggtgggtatggtgggtctctatgggcccacattggaaagacttcaaggaaattgatgtgaaaaagtacattgacggtcagtctgcattgaggatcttattgttaaaaaaataaatatataccatgtaaacatgcaggcacgcagatgtgtaaatgaatgtgcatgtgaggcagtgagacagactgacagggaaggagagagacaatgagagagacagagcctgagagatgtggccaaaccacctgatgatcagatttggtggtgggacattgaaaatattttattttctcctttaagtaaaacttaaatattttagaatgttcatcttatctttacaattataaaaagatcacatcatattaaatactatccatttaacactcacctattaaataattgatgtatatttcctagttttaatttttataggcattttattacaaagccaaaaatatgtaatatattaatagtaaaagaaggaattcaaaattaatgaaaaataaaataaaagatctagtcccttcacccagacacagtgccctccccatgagatgctcctcctcctgtcctggccctccgtgtgtagcccatgtgccctgagggctgggtggggaaacttcaagccctcagctcctctgtgatctgctgtccccannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngaagggtttccagagctactttgatctattttttgcacacacattttttggacaaaaagatttcacatttaagctttttataaagaaatttaataatatgatttcctatctagtgagcaaataaataatgttatgttgatttgtacttatttgggccatttaaaaattgtgataaaagataataacattaaatttaccatattaactatttgaagtatacaactcagtgatagtaagtctattcacattattcatttccagaaatttgttattgtaccaaaccaaaactttgtgtccattaaacaataactccgcaatcctcctaactctcatcccataggaatctctatcaactctctgtttctgacggtgcctattctaggttcctcatacatgtggaatcgtacaatatttgtacatttctgcctggctgattacatttagcataatgttgtcaaagttcatccatatagtagatctcatttcttttaaaggatgagtggtatcccattgtatgtgttgatgtttacttgtagaataagtaatttatatattttttccatgaacaactaactgaaaggcaaagcaaactactgataaaagagactagaactggctacaaatggaatcctggcaacgttatttaactattccctaatttaaccaattttctaagtaaaacaatgaaaagatgagtaggagttgctgctttttggtgtctcacacgctaatcatatagacaatatgtccacagagagcacagtaaaacaattagaacaaattcctaattgcctgttaagaaatgctggaatattcaacatgaattctatcctatgacctgggtgtacatagaaactttgtttattcaggtttgcctcttgtttaactcagcatattatgatgaattattattgttattatattttaaagtaagatggagtcttgctctgtcacccaggctggagtgcagtggcacgatcttggctcactgcaacttctgcctcccaggttcaagcaattctcccacctcagcctcctgagtagctaggattacagctgcccaccaccatgcatggctaatttttgtatttttagtagcgacggggtttcgctatgttggccaggctggtcttgaactcctgacctcaggtgatccgcctgcctcggcctcctaaagtgctgggattataggcatgagccatcacaccttgccctgatgaattattttaatggccctagaaagccctgcatttagttaattttccatcttgctttattgatctgaatagagcatgcaatttttgtgtgtgttgggcagtttgtgttctgatgctcatgggatgccacagaagtcgaggaccaaggatattttcttgtaaaaaccaacatttgcccactttagaaaaccagagaaacaaaaaaacggagatttactttcctccagggagatgctgttttgtctcctgaagaagcttggtaagcctcagtgtaggatgtaggtgatggtttcacatgaggcatgttttaaaaatcctctctgatactcttttttatgtctggtggccaatctcaatcaaaggaaggctttcccctgctttgtgatgagtgactcttggcatgaagcaagttaggttcctccactcagcgatgtggtttcagtctcagactttcaatggaaacaaaaaggaaggaaggaaggaagaagggaaggaaagtttagatatataaagaccaataatcagattgtgaattttgactcaccatatttattcatttaataaaaaattattgagtacctacgatgacaatgcaatcagcagagtggacactagaaatacaaaggtgaaccagataaatctctggccccatggagccagacagtcaggagcagagctggtcagtgacagagcagttgtcctgctgggaccttagtgctgtgctagggaaggttcctggtgctctaggatgaggtagggatagaaggtcagagaagacctagacatcaactaactgcattacattatttcctttaagtgttcaagacttcagcaattcacaggttcaaattcaatcatggatcaggagttgtaataaagatttctcatgtcattctggcacctggcaactcattgtttattcaatcagagattatcagaataaaaatcaagattgtgtcattaaacaaaggaatgagttaaatagtaagtttatgatattaatttactaatttgttttacagattgtaagtttactaataatgcaatgtttcgttaggtgcttatagcaacattacttagaaaaatcacaatggctgaaaactcactgggaatctgagctgtacttctgtaatcaacagatatatattgggtgcttactaagagccaagcagtgtacaaggcttggggacacagaaaacaacatctctagagttctccactttctgtctatcagtgctcactctctaacccccatcagcccacattaagtaggaggcatttaatgatggtgaatggactttgatctcaggtaggtgagatctgttacattaaccatgattttttaagtccttctccccttttaaaacttcttatccagctgatttcctgggatcctattttttctgggtttggaaataggtcttctaatgatcacatgtctcccagcttgaatttgcagccatactagtcagggaccatattcagcatatcccctgatcagctggacataggcatcaacttgctgacacgagcaccgactgctcagtcctgggcactgacccatcgggatggtagccaggccgccctcagtcctctaagggtgggattcatcaccaccaggtctccggctttcactccggtctgttaattccatccactgatggatgcagatgtcagatccctggtacctccttgggcctttgcttcctgcttggctgtcctgatgttgctgagcccaggacccttcctccagatctgtctacttgggctcatgttacttcattcttttcatggcctgttctggtagccttggattccagaatctttcccctaggcttttactccagaatgtgatttttccatcattagcccttgttaacccaaaaactatctgtaacaaatccttctggttgacctagaatcagagaaaaataagtcaactcagcaaggtaacaaaacaaaaggtgaaatgaagaaatccgaactctaatgcaggtaagagtgagcccaatgctgttgctttaaagaatggggtgcacggcctttccatgtcccaggcagctgtctctgctactgtccatctgccctgcctcccactgatacacacttactcattgttctaacatacataagtttgtcccagtccaagccagaggtagacagtaaaaacagacaactgcttaatctggggagttatctttctagaagactcaaatacacctgcttttaattctagcttagctattcactcaccagctttacagtccttgtgaacttacttaccctctgagtctcagtgttctcatctgtcaaatagagttatgatatccatgtattcaaaaaaaatgcctgacatatagtaggtgtgcaaagaataggggctattttattaaattattcaaaagtatttttattaaagaaagcctcttggagacatgacgccctaacagaaaaaaatgaacctagggtcaggaggaggcatggggtatttgaacaacatttcattccaaccacaaagccgctctgccttcatgttgttaccttgtcattctaaactctgtgcacacacaaacacaggcacattagttaacacggtaaatataaaaaataaatgaaattttaaaacacacaaaaagcaactcaaaattcctgaacagatctgctaatcattgtggatattctctgctctctgttgagtagccagctctaattagtcggccctgtacagagctagacaggcctcagtcacttgccaagaaacaaacagtgatgccccaagatccacaggtgtaaagtcactacaaggtgcccttctaccacattaggatgtaaaatatctgtgaaaaattgagtcactgggagagtgagaaataacatagaggcctgtacatgctgaagcttccttcgatctctgcctgccccacccctgccctgctgcaggcttggtcctgcctggtccctgagctgctaagaatgaagccaggtgtccactaggcccttagctctgagagcgggtgtctggtttgggtttgatgcctaaggctgctatccattgtccagggagctgcaggcccctctgtaaatctgtgatctccgtccagatggttgagtttcctgcctggttcctggtttacatcgaaatctcatctaagcctagacttccttgctctctgctgatttgacaggggtctaagcaccgagcctggctcctgctgctcttctccggtgggctggatcctgcctctccctctctctctctctctctctctctctctggtctctgtctcctgtgaaatgaactttcctggaaccaaaaggaactcatccatcatatatggtcagtgtcacagagactgtggcctcaacacttaaacttccaagaccctttgaaactgatgccgtcgctgtcctgccctgggtgtcacatccccagggctttgacattaggatgactgctccttagctgggggcaggcctatcatcaggttggtttcctcatttccaaccctgactattacaggctcactctacattccctgctcagcaggtgcctgcagacttgcccttgagtcccatcctccttagggactaggatcactttctctccattcatgttcttattcccatgggttcattttataaaaattttatccaatatgtgtgatgtacaaagctccgtcttggaattcctgagagatacatcctaacatcatctcgatccccaactttccaaaaccacggtgacacttcacaggagtggctgtgtgcagaggagaagacaggaagcagcaagagcagccctgggctgggtgcagacgctcggcacgggagtgggttcctcagctggccgtgtgcatcttgtaccatgcacaccatcctgagtcttcacagtaaaactgtgcaacagaaacctgcaataaggctctctgatttttttcagaccattacaaaaaccttaaacccacatatctctcatttttacatttcatatttgctttctgtttcacacccaggatacaaaagcgaattcattttcaccagttcattgtcatgacttgggcgcgatgaatgaaatgtttagcagctgtgatacgttcgctgtctttgtgttcagttctagcctgagtttccccaaagcaaaagctatctttactattgagcatttatacaggcctcatggttgacaagaatttatattctttacacatgaaggtatccttaaagaaccaaagtgaactacatctcatatcacggagacagtgtcatcaagctgacagaggaggcttctgggagaaccaacaatgctgacatcagccagtctcattcttatttctaggagttttgaggagcttatcataccagcggtttttgtttttcccctttcctgaatttttattttacttctaattcggcactgaggctataattttatcatttggggcatgatttcctcgaataacatttaaaagatgcaggtttctactcttcctcctttcttagtagaagtttccatattcaaagcagtcattttttaatcattttaattatctaatcatccacatcatagtaaagttcttaatatagaaattgatctggataatttttgacttctaagcctttttacttttgataattagttattttaaaagtttcctgctaaatatcagtctttagatatttttgaatctgaacaccaagtgactttggtcagagatacagagaggaaaaagactgaatttaatggaaaatatcttttatgtacctactatgagcatggtattgtgctaagtgacttgggatacaaaaggaattcactcgctctctcattgagcgtatctattgagccacgactacctgccataagcacctgatgtgttgtgccaggcacctgctaggtgctggtctatgctgggaaatgtaccatggatgatactatctctagagcttacaataaacttgattaaaaggaaatgaagttgctaaatacctgataaatgtacatgatgtgactagtgttctacatggattattgtttaatcctcaaagccattattacctgtatcttttctgggaacctgagacaagagaggctggtaacatacatgagattgcactgtaagtgatgaaatcaagacttaaatccagtccttctaactccagcactgttaagtggtgaaaccaagacttaaatccagaccctctaactccagtgctattgggtagagaagacgggacaattcctctcttccatttacttattatataaagaaaaattgaagtaagaaataaaaaagtcaataggaaatgtatttcctgagctgtctagacattcccatggatgagtgtgtactaactgggataatgcgggcaaggagatggggcagtatttgaaacagacatatactgtgttcaagtacagtggttctaaccacctaaagttgttaaagggccattaactttgagtgactcaatacactggatccccagcagacctaaccaaagtctctgtcctggccagtgaacagtgagacttgtatcagctaatctcacacagacacttcaagaacgtttctgggaatcgaaaggtctagtacatgttgagatatcctctcagtttcttcccctttatgaaaatgtggggaagttatttgaagactcaattaaaagtcaaacacatcttgtatggttgagatttgtcctgcatgatgctgcatatctcttagacaagtgatcgctatgtggcattactacacataggtctaatacaaagaggtacctcttggttagaagacaagggtctaaaaaccaaaagatacctctcactctcacgtataataacctaatatttaagaattttacttctccttcctgtaactctgagctttgcttgtaggagtgttttaggtacaaataaaggaatgcttaaagccggatgcaaaagtaattctcttgcattggaaattgaatccgcctcctaatcatttcacggtattcattacaccgaaacagctgaattgttgtggtgctacttgaagtggttaatactaactacaagggaaaacagaaaagctacataatgggtataggaacagtatggataaaattcagatgatcccaatggagatttttaaaatttcatattttcatttttttttcataagagtatgtcaggatacatacacttcaatatgcaactaagatggactaaaatggatccgatttactttcctgcctaaaataactaaaaaacaaaatgtataaaacatgcaaaatatatgaagcagctgtttgctagacaatgaacattaagcaacaagggacagtgatccttcaaagacaagaaataaacagaaaagccagatggttgccccgatttactactagggaaagtttacagactgtggtgcagaaaagggaactcgggcagtgcccagaatcttgcagagttgagaagatgaatctgggaacctgcagaggacaaggcagttatatcttccaggacagagctgtgccagagaggagaagattgacagagagagagcccagatgtctgcagagaaatccctagtattttcagagcagttctgcatgtgatgaaactactcaagtgtggagaaataatcaccaaaaagaattagagggaattatcctcagagctcatacagggctggaaatagtgcctgttcccaaaggcaaaagtgaaaaacattataattcatagagcatctgctagaggactgagaagggtgctgtctccagggtgggaaacattaacaactctagattgaaaaactgttttgatcccaccaaacgcaataaaaagtaagacccaaaaggatcaaactgaaattaattacatcccagagcaaaactgaagaacatttctagcaatacaaaaatatccagcacacaagaagataaaaatccgttattttcttccaataaaaacttactaggaaggaggaaggaaggaaggaaggaagagagagagaaagagagagagagagaaaggcaggaggagaaagggaggggaaggaaggatgaaggaaggaaaaaatgaaggaaggagggcagataggaaggaaagaagatacaactcatattaaagaaaaaaatccctgaatggaaattgacccataattaacaaagatgataagattagcaaacaagtaacttaaaacagaattatatcccaattcattatgctcactgtactagaggaaatatactagtgaaaacactgaccaagtcacatttagaggatataagaaagagagaaaataaacttctagagattaaaaatacaaggtatgaaatgaaaaacacactggatgagatgaatgcagattttatattgcaaaagaaaatatttgtaaattttaagacagggcaatagaaataatccaacgtggatagaaaacatgaagaaaagagttttattaaacaaaaagtgcatcagtgagccttaggacaacttcaagggtcttattgtagctgcaattagagtccctgaaggagatggtaaagatagagttaaaataatttaaaatttgagggccacaattaattctttctttctgcttgctttctttctttctttctttctttctttctttctttctttctttctttctttctttctttctttctttctctttttctttctttctttctttctttctttctttctttctttctttctctttttctttctttgtttctttgtttgtttctttctttcttttctttctctctctctctcttttttttttttttctgagacaaggtctcactttgttactggggtttggagtgtagtgacacaatcatggctcaccacatccttaaccacccaggctcagacaatcctcccacctcagcctccgaagtagctgggatcacagggacacaccaccactcccagctattatttgtattttttgtagagacgggatctcccctactccccaggaaggtctcgaactcctgggctcaagcaatctgcctgcctcggcctcccaaaggatgggattacatgagtgagacactacatccagcctcgaaattttctacatgtcgaaaatgacaaaacaggaaaactcaatgcacaagaaaccgtaagcacaagaagcttcaggaaacgacaccaaagcacaccaattacatttctgaaaactggagatagaagaaaatctgaaaactgaccagataaaagattttcacaaataaaaagacaagaatgaccattgatctctattaaaactaacaagtaagattttgggtaagtaagatgggttagatcaggtaacagttaaagaagacataatcaccaaggaaattcatggaaactccgaagaggtttccatgagaaccatgtaagtatcccaggattaaagagaccttcagaaaaattaccaaggcttcagaagatttttgccaaaatgctataatttggttagaaaatttgtggcacaaatacccagtcaacactggattcattgtttgttggttttgggagacagtgcatagttactactataattatactttgttaaatatgagattcacttatatttcacaattaaatttttttttcaaaatgaattgtcaagcatgttttttctccataacttctttgaagaactggcctctaattaataaagtagctgactcctggttcccattatccatgctaaccacccccttttgtgtctgtctcccacagaatccaggagctccatgtaggcagagagcatattgggtctctggtgggaggcagagccccctcccccacagaagctgaatatgaaggataccagggctggtggctggtgcaggggtagaggcgtgggtcattgctggaggaggctgtgtgggttggtgtttctggctgtgtccagctccaaatctgccggcctgatactctggctgtcctgaagctttcacgagcacctcatgtgctttgagattttctctcctgctcacacaagctggagtggtgctgctattaaagctgagaacgtcatctgtctcctacacattcacggcggtcactataactcgctgacctccccttacaagtgctttcctgagtgagagaaaggagctccgtgcttggagggagaccaaacaggcagggtggccagacctgccccctattctctcctggatggcagcgcctggctgctttgctcagaagctgagttatggagcaggaggggctcagcagggctttctgggctccccggttcattccaggtgccctggtggtctccactgctggcttgtctacctccctgtgttccctcagggctcagccccaggatctgtgcagcccccactttcctcagcgagcatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgctttagggtttttagtatacatctttaactttttacagtccaccttcaagttatattataactcttcacatatagtataagaaccacctcctcaattcaagcaattctcctctcaagtagctgggattacaggtgtgcgccaccacgcccagctaattttttgtatttttagtagagatggggtttcgccatgttggccagggtggtcttgaactcctgacctcaagtgatccacctgcctcggcctcccaaagtgttgggattataagcatgagccattgctcccagccagaacattttaatagcatacttcaacttttcccctttcctctttgttctattgttgacatatgttaactgtagacatgttatgaatcccacaacacattgttttcattttttttacttcaaatagttgattattttttaaaatgtttaaataataacaaaaaacttctatacaattacccagatattgatcattttgattgttcttcattcctttatgtaaatgcacatttttatctggtattattttccttctgcttgaggcagttcctttatgtttcttgcaggttatctggaactgtaaaggctctaaaatgttagccaatattttaatctaataatttaacctgctacagcttcttgggtgtttgtggaaagcataagacttctgtacattggacagtttattacacacagtattagcaatagacacagtattatcgacattttcgtgttagttccctaagccctaattttccaagagagacataaagaaggccagatgacccctatgcatacagtgggttgaactacaaaagaggagcccagagtttacgtaatgcaaaatgtttataattggcagcaaaaattcttgttattttctctgaagagtgaaattatctttattacacaggacaggaaacatgcctaccctttgctcaagagggagatattatctcccaatgcttctaagactttaccttccaaaattgtctgctatacaaacattatgaacaccttagtctggaacaaacaatactcagttcttcacttgcaagacatggaaatgtgggagaaccatggaaaattgtctcctaatgcaggtctgctggtgatgaacacttttgtatatctgaaaaatgttaattttatcttcatttttcaataaatatttttgctgggtaaaaaattctatgttgactttttttttctttcagtacttacaagatattgtccaactggttttatagcttgcattgttttggaagaggcagccactgtcaacatatgtttattattttgcatgtgattcttttttctctgattggtttttaagattttctctatcactagtttccagaaatttgattttaatgtgtctttttgtaattttcttcatgtacctttaaaaaatatatgtaacataaaatttctttttttctttttctttttttttttttttgagacagagtctcactttgttgcccaggcatgcaattgtatgatcttaactcactgcaacctccacctccagagttcaagcaattcttctgcctcagcctcccgagtagctgggattgcaaatgtccatcaccaagcttgcctaatttttatatttttagtagagatggagtttcactatgttggccatgctggtctcgaactcctgacctcaggtgatccacccacctgggactcccaaagagctgggattacaggtgtgagccaccatgcccagcccataaaattccattctaaccattttaagtgtaccattcaatgacattaagtacattcacattgttgtgcaactgtcaccaccacccattttcagaacctttttatctttccaaactttccaaactctgtattccattaaacaataatttcttgctttctactcacccagccactaataacctctgctcaaccatctgtctttagttttcgactattctaaataccttatataagtagaatcatactacattttctttttgtgactagattatttcatttaacatcatatattcagcatgtcatcatatgtatcagaattttcttccattttaagcctgaatattattccattgtatgtatacagcaccttttgtttatccattcacccactgatggacaagacctgggttgcttccaccttttggatattgtgattaatgctgctatgaacatgggtttacaaataactgcacaagtccctgcttttacctctttaggtgtataaccagaaatgaaattgtttgatcatagcgaagtctatatttaattttgtctggaacagtaatgccattttgaacagcagctgtagcttttccatgtttcttttgcttgaggcttattgacttttttgttcatgtgggtttacaatttttataaaaaattttggccatcaaatctttaattttaaaaaatttctccagtcttgggtagctcacatgcacacactgatcagtgctcaactgaatacaccagggggctctgcaaatatgtgggttatctctttgttaattctttcctgtctgccgggattcggtcctgtgaaatagttgtcttgttccctgcaggtggccggacacatctcctcaactctgcaagattgctgggccctacctgagttcttccttccttcaccacagtctgcaaactttctccaggcagtaaatcagagcaatcatagggcttttctgtctctgagagatcacagccccttgttgcttaatgttcaatgtcttacaaactatagtttcatatattttgtctttgtagttgttgaggcaggagagtaaatctggtccttgttaggtcatcttggtttaagtggaagtgcatcctaaaaaactcttatccctagacatgaaaagatgacaaaatgaccaatgagatcacctggatttcatccatgcttctccaatctcccatctcccattagctagcatctctatttttccttggtacttaggattaaccactccaagtagaactacaacgatctagctcatttccctgtaataaagtaccatgaaatgactcagaggaaaattccatttccaaatcagtaaaagcattttgtatttgattttaaggattccttcattggtatctaaaacactctacaagcagtgctctgagttataggaatgagaagcaaaactcttgaatgtgatttcattgcatgtggttgtgaaatttatctcttgggttttagatccatgtattctgaccaagaagtaccacttaattttagacccatttggaggaatatcatatattggtcatttattcaagatatatgcagcattatacaggacatctcaactttgcaaggtgttgttagttgatctttaactgagtcctcaaataacttctccaacttttcataagagtctctgttcaggatgatgacatggtatgtgatagctctagtgaacccctggggagcccatctattactcaatttttcttgatagcaatcagtgtttctggaaaacatagatgacctgtgatattagtaaatccatgaatgataacatggagagagagacaaataaagataatcaatcccaggataaggatattaagcaagaacaaattgctcttctttcattgtgagtggaattcaatacaataagcctcctactaggtagctgatatgctgatatctgtgctcattctgagagtcctttcaaatatctgttctccgaaacttttcatcaccaacctccaattttcttctttctagccaaaccattaatcagtcttcataaatctattcagactcatctctgaggctatatggatgaggggtgttttcaaaactctgccctctgaaagagtttttcttctatatgtttgtggtggtttgaatgtttttttcacctccaaaactcacgttgaaacttaatccctagtgcaacagtgttgggaggtgggactaatgggaggtgtttagatcatgaggactccattttaatgaatgaattaatgcagctatagaaaaaacttataggagtggcttccctctcttctgttcttcgacaatgtgaagacacaatattccttcactctagagagtagcaagggaacacaatcttggaaccagagacctaatctactggcatcttgatcgtggacacctcagactccagaactgtgagggaataaatttttgtcctttataaattacccagtttcaggtattctgttataatagcacaaaacaaactaagataatgctctatcagagaaaatggagcttatgaaattataaagttattgcaaacagttttattcaattcaagtcaattaatctttattgagttcctactgtatgcatggaacttcaggctggtgcatgtatattttgtaggatcttactgtattattttctccttagtattttttagtagggagcaatgatgagggcataggtagtttgagagagtagtgagagagtatcaggaataggcataagggattgtgagtcactcgttcaaagaacttgtttacgacatgagaattatcaaatgcctggtttgacatataacactttgacctgatgatggattaccatggagtattccaaatttcaaagctgtgtggtatgatagcactagtttataatgtgctctttaagttgtatgctcaattatggctttacagttagatgtttattattcaccaaggcgtagtagaaagctaagagccacttagtgaaaggacaatagcagaaaaaggcatacttttgccttaaactatgatgccttcgctgtgaatttttttatttattttattattattattattattatttatccaggaatgccatggattccattgagtcttctgacccattacagattctttgagcatgattggatatgctagatcataaaggtcaggaagcaaaatgaccaatactatttcctgactcatctggaaaagattcttttgcccagggtcgtgcttaaagctggaagcctctcggagacatgggtaaatgattcatggcagtgtactcaaatgtagcaaacactgcctccaaaagcagaagggactcacttggtgttagaacttgctgttaggagtagcggaagaaatggagggatatcccggcaggtgccaggccactggactccctgaaatttttctgaaatattgttccctgtattttcatggtatttatctccctctggcatatatatatatatatatatgtatatatatatatatattttttttttttttttttttttttttgagatagagtctcgctgtatcgcttaggctggagtgcagtagagtgatctctgctcactgcgacctccttccccctgggctcaatcgattcttgtgcctcagcctcactagtacctgggactacagacgtgcaccaccacacccggctaatttttgtattttctgtagagacggggtttctccatgttggccagcctcgtctccatctcctggcctcaagtgctcggcccgccttgaactcccaaagtgctgggattagaggtgtgagccacggtgcccaacctgtatttcaatgatatttaaagtagttgcttcttgcttttcaaaccccgtttgtatattaccggcaatgaagtaggaatgctaatatggttcttgtttgaaagtcggctcactcatgtaaggaaggaattgtagcttcaatgaaaagataaataatttcatcagatacgtgtggtatgtgtgggagccggttttgaatacacagagaagtttagggggtgtttgaaatgctgaggttcttggagtcctttgcatattccagtaagttcacattcttggaattattttcctttttttttttttttaatccagatcatatgtttaacctaagttaccaggaggatctataaattcaattaggggccacctggatctaaatttgaattcaatttttaattaccctggggcataggagatactgttaggaccatgatagaaaaatgctaaacttttgtctgtctcaagaagagaatttaaggatttgagctcgctgttccctttgcttacacttatttagacaaactctaggccttgactttttacccgtcttcctgttctttggaaaattcaccatgacactctaaaacattgtctttaaagagcatactattttaagtgccaaaaaatctctaatttgatttgctattttgccaccttgtgtactgtaaatctgcagggtctcatgcctggtttttactgccttcaacctccaattacttagataataagtctcagaatccccacattctcctgagatgctattgccaacaatccaggccttattcacactttctgtatgacttaaactcctcaattgaaaacaacaaaaaatgactttagataacttaggtgtaaataaacttgtcggaaagttactcttagcttacaaaattgacagtgaggttgaagaaccaagctttataaaagggacaagcaaaaaaggaagctttaggcattagaaccatctggctattccttctatgctataacgcatagccatagccacacattgtgaccatctgtggactcagccaccagaaatctaatctaacaacaaacagctacttggaaggctgaggtaggagggtcacccaggcctcaggagagtgaggttgcagtgagccgtgattgtgcctttgcactccagcctggaaaacagagtgagactcagtctcaaaaaaaataaatacataaagttcatggctcttgaacagcttttgctgattagaaccattgaactaaaagtgcagtatttagtattgtaactcaggcaggtgctaatataagtggaaaattttccttactaaatcacaaaagggaaatttcctgctgtttgctaactcttgggttattttctgttttagaagtttggatgatactggcagtgtatttgggatattaagactcaaaggagcaggtctgtggaatagcccagtttcaggatggctccccttgtctctctgctcatcttgttccacgtcaaacaatgagtgtaagagtgggagaatcattcattgtagaaaatagcatgttaaaaaatattcattgtaaatacataaacattgtggtggtattttggaccatttgatagaatagaaaaataactgcattatgcaaaaattatttttgtaatgaaaattgttgaaatagaattgatatttcaaatataatttcttcaaaaggaatactttatatgaaatttaaaacagaactctagaagggtaaaatatttgtgaattatctattttgccaaagtgcttgaatttttattgcttttattatttattttgcttcagtttcttgccattctccgagagaattccaatgactggtcatttttagatagaagtgggttttagcttgtaagggctggtacactgatttttttttgattgaataattaatatacttgtggaaatctctgcttagtttaggtaaaactctggttttcttaaaaattataaaagatttgtaaaacctagagattcgagtcatctgagcttttttcaatgtttaaatacagaatttagatgtatttaattcccatggccaaagggtagagttagaaagtgcaatacctttataatttttaatatttacagtctattacttatatattaattactaattgatattaaccacatgctttttaaatatgaagataatattgtgagactaatttttgtttattgtgaatcctaaatactgtatcaaggaattttcagattttaaaaattacatcaggctgggcctacagtgtaatcagtgttctctagttgatgttcccattgatatgaaaggtagtatatgttgcatttatcagagcgaacctcttttaggtttactttaaattttattgttaaatgcagagacagattacagttcagcatcttcccatccagaagcatgataagattcctgttgtgttctagaactaactgttgcacgtaaaatgttgagtgaaaatgcaaatagtttataaccttttaggatttttgttttatgcagaattcatctctctaacttgtataccttcttgattcctttaaaaaatatctaacacttaacacaagtatgaatagaactaaactagaaactgaattaatcactaatttaattcaggcattactaattaatgtacctatcagtctattagggctacggaaggcacaataccagtcctgattctaagccactcttcatactaattggcaaagcatgacatatctgtggtagagagtgagaatataattcaaatctaaacaggtgctatagctattcaaaatttaaactttgatcaatgggatcaatattcatgacatattattaagcaaaaaacacatttatagtagaattcaatttttattctgtatatattatttattacagataggactatatatgttttatagagaaaggacatatataaaggacatgcaaatatatataatatacataaaatattcactgaagtacagtatctttggaggttagccagtaattagcaccttttcctaaagttataagaataacatgctcaagcttgttttcccaatgtctaacagttttggatattgaaactcgtgtatctaggaaaaataaattaatcaccagctgtgcagtgaagtgtgtgccccaatatttggtgttagcatccaacattcatgctgcacattgcagaaattggtagaaatgcccttcagaatgctcttgctactgaaaatgggagaaagatgaaagaggacagtacagaattaagggactatttacccaattttcaacgtttagctggaaaggaacatggaatagtctaaaagagtgggatatataactgtaatgagtttgtaagttatattgttgcaaaatgatgaacagagttttattgttttagaggtttcctgttgatctttgtttttttttaaaaaaacctgacctcagtggatttcacggctgtaggatggtggccactgccacctacagaccagaagtgtgaagtggcgtggctcttttgcagtggagtccctcagagactcggcatcaaacgttctataaaaagcaatttttcaagcaaaagttgaaagaatttcaaatgaaatagtaaatattctagagaaataaaaaatgaataatcacattttccccagagatcactctttgacagtatatagcatatatattagaagattaacacctaatttatattttgagtcagacatacacatctgaccacaagactggacccacctaatatttcctttattcacctcattttttatgaggtgaatatgtggaggggagaattacattttgcaacagaaatttgcatccacaatgaaatacacactcacacagacacacatatatatgattgaagggacaagcagatggtaggtaggtaggtagatagacaagcagacagaattatagatatgaaaggggggaatctatacagacaaaatgtttatatagattatctcttagtattagaattacagattatttctgctactttgattcttcttgcttcattttattttaaaaattctacaatgaaaatatactccttgtgtagggatttttttctaacttgaaggcagagctttgaaaatcaatgcccgcataacataaagcttttaaagtcttgctccaggtagaaaagaaaatactaaatcttcaaatattcctggcatacattattttttaatttaaacatacacttaaaagcacccaattttattatttttccaaaagattttagaaaattatacacagtaatgcctcattccattgactgtaagagtttcatttctcatagtaaccatgtatactaaaatttaatcagtaacagataatctattatgtcatggtattaaaaggttacataggtaaaggttaataatcagatcgtgaattttggctaaacatcattcattcatttaataaaaacatattgagtacctaccatgacaatgatactagaaatacaaaggtgaacacataaatacaaaaacaaatataacaaagaaaaaaaacaaaaaaagtcaactcaagatttctgggtagttttgttgaccattgtgcatattttctgtgcttcgctgaatatccaactctaattagttgtctcagaacagagctaagtaggactccgtcacttgccaagaaacaaacaatgatactccaagatccacaagagtaaaaatcactgcaaggtcccctccctgccatttcaggatttaaaatatctgtgaaaaatggagggactctttttgagtcattgatagagaaataacatagatcctaaggtttccttcaacctcttcctgccccacccctgcccctctgcagaattggtcctgcccagtctctgagctggtgagaatgaagcctggtgtccactaggcccttagctctgaaagcaggtgtctggtttggggtttgatgcctaacactgttatccattgcccagggagctgcaggcctctctataaacctataatctgtgtccatgtggttgagtttcttgcctggttcctggtttatgtctgaatcttgtctaatcctagattttcttgctctctgctgatgtggcaggggtttaaacaccaagcctggctcctgctgctcttctccagtgggttggaccctgccctctctctctggtctctgcctcctgcaaaacgaactttcctgaaaccgaaaggaacccatccatcatatatggtcagtgtcagagactgtgccctcaacacttaaagtttcaagacctttggaaattgatgtcattgctgtcctgccctgggtgtcacatcccccagggatttggcattaggatgactgctccttacctgggggcaggcctatcatcaggttggtttcctcctttccaactttgtctattagaggcatatcccactttccccacttagccggtgcctacagacttgcccttgagctcccatcttcttaggaaccagggttatttgctctccattcactttttcattcccatggctccatcttgtaattttgcccaatgtgcataatgtataaagttccatcatgagacattcatgagggatatatcctgaggccacctgatgcccagcttgctttccttccttccttccttccttccttccttccttccttccttccttccttccctccctccttccctccctcccttccttccctccttcttttttttttttttttgacagagtctcgctttgtcacccaggctggagtgcagtggcatgatctcggctcactgcaagctccgcctcctgagttcaaaccattctcatgcctcagcctcccaagtagctgggactacaggcacccgccaccacgcctggcaatttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaagagaaagaaggcaaaaaggaagaaagagaggaaagaaggaagggagggagggaggaaggaaggaaggaaggaagaaaatctggaactttccttcagtagatactgtctcaagtttgtaactcttgacccagaaagaagtttaacatgcatagatgttgttctgtgtgcccatcccaccatccgacccacccatatatccagtcttataagatgtcttcagggacctctactgggggttccttgacttgaagtagcaccacctggagggctcatgcttgtctggtggcactttaatgtacctccatccaggcgcaaaagtcactagcattcagcttggttagtaagtcccatcctagcagtgacacagggcactcaagcaaatattttaaaaaccatgttttaggtgtgatttgcccatttggtgatctaaagtttagagaaatggctttttcaaggtttctccagggatcttagttattagcattgttttcagttaactagaaaaatctaatattatgaactgaataggtagccccagtatcaaccagaaaatccaaaagatgatttcctgtcatcttggtcacctggggttgcaagtggaaaatcatttttttttttttttttttttgagacagagtgtcactctgtcgcccagactggagcgcaatggcacgatctcggctcactgcaacctctgcctcctgggtttaagtgattctcctgccttagcctcccaagtagcaattacaggtgctcaccaccatgcctggctaatttttgtatttttgtagagatggggtttcaccatgttgggcaggctggtcttgaactcctgacctcaggtgatttgccagcctcagtctctcaaagtgctgggattacaggcatgagccaccacgcctggccggatgtggaaaatcttaacaggatcagtaaagtcaagtgcccaggcttcgtcattcctcatccctgttggtgatctcaggcatttggtgcactgcctgatgcctatcttcttcaagagtcttattttggttgaggcattctattttcccatggccctccctcttgtttacagtaggcatattagtggggtcccattgcggggtgtcttatatggtttggattctgggatgggaggctgcctatgtttaatcaaggtcttcctacttgtggaccatgggtcagttcctgggtcagtgccaccgccagcaaggcagcttattgtttcactttctggacttgattctgccactgaaaatcttaaggacaatgtcaaccagttgaaacatcagcatccccagggtctcctctgttttctgtagtttcttctgaatatccagagtgctttggctaatatagcttatatttaccatttttaagttctcaggggcctctggatctacatcagtgtattgcctatacattttaaaaatcctttccagaaattctgaagttttctctttaggttttggtttcatctcctgcattttatttagactcctttgttttggaactcccttatggagcactgctaaaaagcaattttggtagtattcaagtctcactctgtctcctgcatttatatttcagtgagcgtcaggtgtggggatggccacggatatcaacagttgctattttatgttttgttcttactgtttaattatagccatcccagtgggtgtggagtggtatctcattgttggtttccttttcatctccctaacatctaatgtagatcacctttcatgtgcttgcttaccatttgtatatcttctttggagaaatgtctgttcaagtccattgcccatttttggttcaattgttaaggtttctgtacttaaactggaagagttctttatattttctgggtacaagattcttatcaaattcatcacttgccaatatttcctgcaattctgtgagtggtcttttcactctcttgacagtgtcctttgatatacaaaagtttttaattttgatgatgcccaatttatctgttttttttcttttactgctagtgttttgtatgacatgttaagaaactaatccaaatccaaggtcatacacatttgcccctatattttttttctaagacctatagttttagctctcacatttagttgtttcatcatttaaggtttatatagcttgtttgattaatatacaacttcactcattcattcatttattactaatcttatatgtctgcatgtgtaaggagatacatcttccagttaggggataattatatttgtttcttatagggaatttacattattttccaaattatatcaaataataacagcaagtaaatatttctttaaaaattctctaaaacaataaaacttgttctgaatgctagcttatagaagtattgaaatgatacaatgtgagaactaaggagttaaaattattagtattatatttgcaatgagttcattattttctgttcttttttaagaatagtgtcacaattaattagtatactaatagaatataaagtgggtttctgggtgtttgcctaaatatgtagcaaatgatattttaattacaatttgagttgctaatcgttgtcaaataaaattttaataaaaaaaaaaatggggaaaacaatgtcgttgtcctaagcagtcctgcactgccctctggtggcctttcacttactatgtcttctcattgttgatttcaagcaccttctgcctttcttgatatttttacatttgactttaatttttttcactctgatagtattttggagtttaagaatgtcaagaaaaaaattaaattatcccttctgtattacaagaatcttaatacacttttatatgtaattcttttagcctaccagaattcctcattatgactttgtaatattttatggaatacaaattgtttacttaatatttcatattgataaggacactgtgctcagtttcctgggtaagttagtctgtaaatttccttacagctttttgaagtataattgatatattacaatgcacatcagccaaataaagattacaatttcacaaatctgggcacatgtatatatccataaaaccatcactgcgattagggtaatatacacgtatacattgcccccaaaagatttttctcatttatttgtaaaaatccattgttagcttgatgtacagataacttctatttggtccttcacattcaccttttgactggggaggaggtggtctgtacagtgaaaaaattgcttgaccttgcttcactgtacagttaggaacccataataaaagggagatctaaagataggtcattagccaggagagagcgttgccaggactgccctggaggagatgactatacgctgaaagcttatgggaccttgcaggtccatgctggcaaaagcctgggcagcaggagtgggagtgggtccaaggcggtggaccaaggagcaaaataaaggtcttgattggactgaattcattgacatgagagagcccactatggatttgggtttaaagtattggatagaccacctgctagtggtgttggtactctgggccctatgatggcttccagataatgaggtcagaatgctcgaatgtccttgccatactgctgagggagggaggaaggagtcagaatgcataaggacaggcatggtgggatggagctatgatgtgcagtctgctcagccatccctaatcttgcctgctgggagaaagcagagctcactcacttcatcaaagagtttaaaaagtgcattggcaaaggcagaggaggcaccagcatcctcaaagagatctgtgcttggccatcctctattgctgccaatgacggtaggggacagtgaagagtgacagggtcagatggctcaggcatcagagacaaggtgcatgcagtcaccagtatgccttgtgggcaggatgggaattagagaatgacccacagggatacatggtgaggggtaattgatcaggggtccttagaaatgaaattgatgggcagcgggatccattgtctctagcatagttatgagacataagccagtaatgacctgactctgagctcctagactgagggagagattggagcccttcaaggcagcaatgtcgtctcaggtatgtatcatgtatcttttccctcagcttccccaaatgtccccaaaatcaaaggcaattactgctgtgaccacactttggaagaagggagatacctacatgcattttctggagaatttcgtctacatgtttggagcagattttgaatccccaacatcagaaaagctgtgtggcccagcaagggctttggaagtctggggatagaagctgccttggcccaaggttaagattgttcagtgggaccatggcctctgcctgtggttactcctcagttccagaaaggatgtatttatcaactagtgggataggaatttttagccactggcggaatctgcacactggtttcctgaacccaggaatttttcttactagggaagaaaggcccaagtaaaggttcctggaactaccccatccctgagagtaaaccaaaggcaacacgtcaaccatggaggagacaggagaggtgagcagtgtcatcaaaccctcgagacaggcaggttagcaattcccactgttgctcattttccttgcttctttggataccttaaaagccagatgggttatggagatgagcagtagatttccacaaatgtaatcagatggagaaggcaacaggagctgtgactctttgtaggatatctcaacagaaataatccatcagttacaccgctgctgcgggagggccctgagcatggcacgagacccctgcccatgaaaccagctgctgagaatgaagaagtgacacaggaaaacctgccagcaccacaggctccacactaagcaggaggtcacagtaactagcccaccctggaggaatttgaatacagcccaactactgactagatggtctcaactccctacactactggcctggcagaaggtgtacacccacttctgaacataaatcctagttaccccagtctctactgctcttctctataattttatgtcaataaattttgaagtttaaataaaatggacaaatcccttaaaaggaaaaaataaaaacaactcattacatcaatggctagtgaagttttcatactcaaagaaaacatcaaagaatgtcaatatcctgtccctctcccatcctagcaggataatttatttttatgattgcaagtggttcatttttagataaattttcacatttttcttcaagcatccaatttcccactactatttttgatttcagcatacttccagttctcatagagaggtgaaggtgctgctttctgggttaaggcccaagcaacattgctatatgagcaggtgacccatggatgctaccatttcctccaggagagataaatattgtggcaggcatagctccttgtttagataaattattttcctcttcttcctggacatcttgcattgggtgtggcccgatgactgatgagttctagcgaatgagatgtgagtggaagtgatatgtttgcttctaggttgtgactttgaaatgtggcctgttccatgtactcttcccctgtctgttggctgaaatggaagaaacttttgtctctgaattacactgaggaagaaaactgcctgtcaagaaaaaatttacacttggattatcatgtgaatcagcagtaagtatctattacattaggacactgaaatttgtggatctcctggttttagtaactagcattcaagtaactaatacagatgttgggctgcttaatacagaatatcactaatatcaataatttataaaataaattatttcccaagattaaatcttaactagatgtggctgagataagttgttttctcccactcaccactgtcttcttccacattagtaaaatgtctgattttatttgggaggatgatggctcagaacaaacatggcatgacatttcccagccttcattgtgtgtcatgggtatgatcgtgtgataacattctagccaagtggctaattctaggatataagcagaagcatcatgtggtagcgactgggatccttccttaaaaggtagatgcttcggccaggcgccgtggctcaagcctgtaatcccagcactatgggaggccaagattggccaatcatgaggtcaagagatcgagactatcctggccaacttggtgaaaccccgtctctactaagaatacaaaaaaattagctgggcgtgtggcacaagaaaccctgtcactactaagaatacaaaaaaattagctgggcgtggtggcacatggctgtagtcccagctactcaggagcctgaggcaggagaatcgtttgaacctgggaggtggaggttgcagtgagccgagatcgcaccactgcactccagcctgggcaacagagcgagactccatctcaaaaaaagaaaaaagaaaaaaagatagatgcttcatataatgtgcccttttcttcttggttattctctacattatgctctttgaaatgcagatgtgatggctggagcaatggttgccatactgaatcatgagaatgagtgccctagtgatgacagatcagtagggtagaaagaacccgggttacaaggatttcatggattacttacattcagacttatatatgagagagagacaaacttttctgtgtgtaaatcattcttattttaaagcctctgatatgtgtagccacactgaattttaattcaaatacttggtcttgaaggataagtaaatgtttatcaggcacaaaaactttggtaagacataaaaggagaacaatctatagttgattaaccttactggttttaaaaaatgaaagaaagaagaaaaaagaaagaactaagttgtgaatttacttatctccctaagaaggacaagctaaagaacacatgaattattcttagagattccagattacaatactaaaggtctatagctgtagatagtgaaggtgcaagggtgtccattgatcgagtccattgttagtttgttggggcttccagaacaaagtaccacaaactggatggcttgaaaaccagaaatttattgtctcccagtcgggaggacagaaggctgagatcaagctgtctgcagtgttggttccttctgggggctgtgagggagatgccactccatgcctctctcccagcatctgggggtttcctggcaatctttggcattcctgggcttgtagctgcatcctttgaatctctgccttcccgtccacacggagttctccctttttgtgtctctgtgtccaaatttcttcttttgtaaagacacagcttatattggattaagagcccaccctgctctctctaccccattatgatcacatcctaacctaatctttacatctgcatctgcatcaaccctatctccaaataaggtcactttctgaagtactgggggttaggacttcaacaaatgagtagtggggatgcaattcaacccacagcaaatactaacattagagtgtaccactataaaaacaattcctacttagatttctccacatggctgaatgtaaagtgaaacagtgagggcactagcagttttggaggctgaagttatttgtggaataatatcacactcctcttctctggggtgaatgccaggagaacatgcagggaataaaaatcaatacctgtcttcaagacagcttcagagccagagggccacccagatgctggaacagaggcccggaattatcaccaacacagagacatagccaaagtgccagagtgcatggaggagggaattatgaactccaggtgaagacgctgagagcagcaaacagtgtgaaaaccaaaacttcagcgcacatgggctttcctcaaagtgtgacagtggaaccgaatgcatggagtccgaacacggctggaaaatccagacctagacagtcacaatggcttagtgaggaaataccatttaaattgggtattcaaggaaatgatctgacaggcggtgatggagtggggaggtggggatttcagggagaaggaataacaggagcaaatccaaggaggattgaaggacggggcaaggctgttgtgctgattgcctcctggggcgtctgcagatggaaagagtgacagaggagctgctggggagccaggggaagtggcagggcaggaaagcagccctttttctgatatttatgccagcttcagaaagctactgtggggtgaggtcgattcggcacattgcatccctattttcaaagctaacttaagggcaggtgggacaccattcaggagtaaggatcttcaaagcctttctttcctttatgggacttgttagtgtgctctactgggcactgccagcttctcctcaatgtcccatgagctgagtgtttaaggccaggcaattccaggctctggttcctcaaagtctgaatgtacctagattactaaggtccttaaaaaatagagatacagggccaggcatggtggctcacgcctgtaatcccagcactttgggaggctgtggggggcagatcacctgaggtcaggagtttgagaccagcctgcccaacatggcgaaaccctgtctctactaaaaatacaaaaaattagcctggggtggtggtgcctgtaatcccagctgctcgggaggctgaggcaggagaatcgcttgattccgggaggcggaggttgcagctagccaagattgtgccactgcactccagcctgtgctacaagagcaaaactccatctcaaaaacaaaaaacaaaaaaccacagatacagaggtctgaagtgaagcctgacagatggtaaaacaatctccaggtggcctcggtgctcagccaggtctaggactttaattcccccatggcatttggagttagctagcatgtttgagaaggcaaagggacgtgagcatctgctgtaaatttggttacaaagttggccaaataagaaattgcttatatgaattatggaatcctatagtcataagatttttaaaatgactactgggaaactgaaaatttatagtgaaattctctctactgctcattttagttcccttcttttctcaaagttaaattataactcaatgtaacccatgaaaggaaaggctgtttgtttcctgtgcaagtctaagattttggggaagggaagagtagaaaaaaaaagatagggaaatcaacatttaatttatgcctactaccatgctagaaactaaagatatataaattacttattataaatatcaattacaacttcagtgattaattttagaatctaaataaaacctgacaaaacttacagtaattaaaaatgtatcaacgaaagaaacagaacagtcaaaataatctttagatggacatgcttagaggatcacattactttcaggacttactagaaaactatagcaatcgagacagcgtggtattgacataacaacagacatatagatcaaagtaacagaatagaatcctgaagtagatccacacttatccatgaactatttcctgacaaagacaaagacaatccagtggtggaagggaaatcacaccttatgtgctctttagtgtcttcttctgcagaacacgatgcttttcaaattcattaattctgtcgctttatccatggtttgtctttcattgctgaggagtattctactggacaaatatactacatgttcgtgttgatggatgtgtgtgctgttaccaatttggagcagctgtgaataaaatggtggtgaacattctgataaaggtctctttgcagacatgtgttttgtttctcctgggtaaatgctgagagctggaattgctgaattataggtcaggaacatgtttacttttataagaaactgccaaagtgattatgccattttaccctctcactagcaacatgagtggtcttgctttgcatcctcactgacacttggtcgtgtttgtatttttaattttagccctgttcagtattgtgtgttggcactttattgtggtctccatttgtttctctcgtgattcattataatgagtactgtgtccctgtggaatggccatctgcatacttccctttgtggaaggtctatttaaatcatgtgtctagttttctatattgcagacaggatattctctaaattctcctcctaaaaactttataatgtaactttcatatttaggtctatggttcatatcaaattaatgtttatgtatgattagtggtagggaggttttttgttctaatacagatgatgaaactaaatcctgaaattgttttacaatctttccctttttttttttttttttttttgagatagggtcttgctttgttgtcccagctggattgcagtggtgcagtcatggttcacagttcactgcagcctcaaactaccaggctcaagtgatcatcccgcctcccaagtaggtgggactacaggcgcctgccaccatgcctggctaatttttgtattttttgtagaacagggtttcaccatgttgcctaggctggtctccaattaatgggttcaagcgatcctcctgccttggcgtcccaaagtgctgggattacagacatgagcctgcaacagcacccagcctgttatataatcttggtagcctctgctggaagaagttatagaacatttctgtcactcaagaaatttcccttgtgcctcttcactttcccacctgtatcagtcaagctcttcctccgacacagaactagtaagatacatatacagcaagcagttgtgagggttcctgggcaagtctgaaatccacagggcaggccacaggaggtcaggctggaagtctgggtaggagctgacccacaatccagagaggagatttcttcttcgtcagggaaacttcaattctttttaaagcctttcaactgattggaatggcccagccagattatagaggataatctcatttatctgaagtgaactgtagatgttaatcacatctacggaataccttcatggcagcacagtgttcgaataatcagctgagactagagcctaatcaagttgacataaaaaccatcactcccttaatccccagaacatctaatgacttctgcctgttattggacttcaaataaatggcatatagaatgtgttttcttttgtgtctggttttgttctggaaagtgctattacttgacaaatttcaaacagctgcagaggaaggatttacttgaggcgatataacttgaaatcttgaagcttaaaccaatacactaatgctcttggctggaaaaagggattagagtgccataaaagagaaaatgtgcccatttgtaaaatttggaaacataggtagaaataggattacagaatgagttcaaaataatacatttgataaaaatgcatagacaaaattataaaaagtatgatacataaatatgtgtatatgatacataaatatacacattttacattcagttgcataacttctgagcatatgactattttaataaaaattttacataaattaaaaatagcatttatagttctgcctagcttataccagacagaatgtcaaatccaagactgactgtccagttcaaaatcagacacgtgctaatcttgctgagaaagtgtgtcagtcaggatctagccaggaatcagaaacacacccataatttgaataggaacaatttaacaagatgagttactaattggcaagaggtgatgagctgctaacagagtaacttaagtgaaagagaactcaagaacacaggaattgcagctggagtgtaagttccaccaatcagatccatgctcagaagatttcaaatcagaagcaagctacttaagcaaggacgctctgcactttgctgccaaaggctgatgcacaggagtctggcttttggaagcatccagggtggcaggaacaagttgctgacaccggggtgtcactacaaatagtgtcagtagaagtaaagtctggagatgtgacagccatcgtctgcatgagggaaaagccaagagaatcaataaaatgcaggacaagtcactgttgagctgcttaaccacgtaccgttgtctatgctgattctctgggcagtttcatgctcctggacttccctgatcattatcatccctacccatatttaatctttttttatgtgccaggtttttgtttgtttacaaaaaatcatcttactagtttttccaagtaccaacttttacctttgttaatttttcatatagtacatttgctttcttataattgatttctgttctttgttatctagctgcttctactttcttctgtttaatttgctattcctttctagtttcttgccacagaagctttgttaagtgattttcatcacttcttccagcataggcatttgaagcaataatgttccttgtaagcaacatcccactagtattactatactgtattttcattaccattcagttagaattattatctacattttactgtgatgtcttctttaacacatggatcatgtagaagtatggtgcttcatttccaaacattatgaattttctatttctctttctgttactgatttctagtaggagtccacttttagcagagaacatagtatgatttcagtctttacaatttgtggcaacttggtttcccctttacggagagtaacagaaaatcaagagacacacaaatatatctgaaaacgagaacaaaatgctgctgggtggtgcggggccgagagcagaaatccagaactgctcaccgcagttctccccctgcatgaaggcctgaacccccttgctgaggacactcaggtggctccggtcaccacctgggccttcagaggggcatctggagccgcccacagaaaggggaggggcgagtggctccttcccgggctcgcgccctctaatttggcggcgtcttctgggaaggaggtggctccagagtgggggtcccgcgggcgcgcagggactggggaggctccgagggagcaagacatttaaacccgggcggtgggggggcgggcggtgtcagacgcccaacgaccacctccctcccggcccgcgccgagcccggccacaccctccccgcgacacccgcgatcgccccccaggactggcggccccgggccccgctcccacccacccacctacgcagggggcgtcctgctcaggcaattcctcccgcgcgccccgtcggggaccgggcggggacgggagaaggaaaagggcccctggctccgggaccagggctccggagggtgccgggcggggagcggaacagggaacgggctggtggcggccccaagcgggagggacggaccgacacgcggccccctggcggccttgcgactcgccgacctgcggaacctcgtcgccgccctcacagccccgcggccaccgccgacccgggccggtccccggggcgtcgcgggtgttaaagggcgagtctacgcggatgtcacggtcgccgcgggcaaggaccgcgaggttgcggccctgctccgaatcccggctgcgctggccacgctcctccacgcgcggggcggccgctccgccacccgcacggcgccccgcagctgctccggctggggattcgcggggtgcagccgcccgggccgcaggaggctgatgtcagcgttccgccgtgaccgcgcctgttctccagataattttaaatcattccattttctcctctgttaccctttataatatgtcagctctctctaataatcatataattattgtttggtaatgacatgacacttgagatttgcttcctctcttttgggttggtttgtttgttttagaattccacttttcactcttttagttatgtgtataaataaacatttattcattcatgtccataatgtgtacatagatatatataattattttaatttgattggtggttgccctacagattcagtatttatccttgacttaatactgtcgcttaaacaaaagtcccacttctagtgtagcttcgagaagcgatccggaatcttcactcattgactccgtttgtcatcctacttcccttttcacaattatttccatgttttacctctacatgtttccaccctgctagctattattataattgttattttttaaaagtagatgttaattcagtgtgtatacgtatacatagtatatacaaaattttgtgctcctcttcattccttcctacatttctgcaccctcaactattaattaaaaaaatcttcactttgaaaaaatccctttaatatttctcatattgcagatctagtgatgaattctctaggattgtggaacatagatttccataaactgctggttagaaaacctggccaaaattaccaaaaaaatgagactaaaggggaacgttttaggcaatctaggaaactattggataagaattttgaattaatgggaatttgaccacttatcacatcaccctggtgtgggcaacaatattttatctgtatgtacagatgtcaaacatattggtcacagcttctcttatgataactaaattagctttttctaaggcttgatatctttaactctaatataaatattgatttagttagctatgttgtttgtaaggtaattgtaacgtagtgcatggcttgcagtgactacacaataaatatttgctgaatggatgaccatgaaaagggtcttttacaaataacaatcactaaattgtggatctaaagataaatataagcatatgtcatcacttgtcagtttccccaaggctgtttgaaaagaaagcatacttgatgagcagagcagccagcagtcacccgcatgctgagtaatggtgaaatacacagcatcaaaccagatactctggtgacactgcaaaagagatctccaagtacaacttcagacatgcaggtttttatcattttacattatcacaaaagtcacactcttctgattctgatgaaaaattatattaatattgtgaatgtagatgccattataaattgcagatcaagcctatgcagacaaacagaaataccctcatcttggtaaagtcatatctatggagaacacattttagatttagaaaactggaattatcaaagctactcttcatttgcccccgaagtatgttaaaaggcaagtgaatcatgtcaagataccttttctctcactaaactaggtaaattttcagaattcaacccagggacccatgagcccatgagatcttcggccttcttggttttttttgtttgtttgttttttgtttttttatggattttgctcttgttgcccagactggagtgcaatggcgccatatcagcccaccacaacctccgcctcccagatcaagtgattctcctgcctcagtctccggagtagctgggactacaggcatgcaccaccacgcccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnatgaggtctcacccattgctctgcttagatgagagtggccccttgaagcaagctctaattgagcccctctgaactgttatatagaagacctgtcctatgctcaaaggatggactgggggacctggagggaagctgaactaaagaattagatgctgtcaggtgtgttcaatcattggaagtctctctcactgctatacatgagttcacttctagcaggttaatgttgcccacagatgttcctctgtaccacctctgtcctggagactgggttttattaaagtcctagaagaatgaacatctggaagatcagctttgtccttgttggaccaggccttatgaggcgctaatagtgacctgttcttcagtcaaattgaaaggagtcaagctgtgaatccatcatacccaagttaagctgatcctagaatagtcattggactcatggcaccttgtatgcagaaacacctccaggaatggaaaccacatggtagaacaccgtagccaagatcatccaaacctcagttggcagtaggcagtacacaccagatactgactcctctaaatggaccaataaacctggtccgacttcacactggtgtttaagagaccacccacctaaacataagcaatgaactgctttcaggttcttgctttacttcttggcttacttgcttgccttctaattctccacctactcatgacttactgtgtattgcctgattgctttctctgcaaacagtgctaacagatctttccttaagtcaccaaacatgattgctttctttcttttttttttttgttgttgttgttgcaccttctcattgatcataatctccagggccctctctggctgtttcaggcccctagggtggagtcttctctgtgtccactcccacctcagccagtgcttgcccctgttggtagcatgaggctgctgcccctcatggaaggagatggagtagagggcctgttgaccagctccctgtagatctcctgcctcgagttcagtaaaggcagtgcctgcctggtagccagtagaaagcactggagctttgcctggccattgttggactggttctgctccctgactgcacacataaccttgtgtcacagatgtccaccttgggaggagaggagatggaagtctgagggcccaggattgacatgggggcagataggagcctctccagcactccctgccatccctcatatgaggaagaacatgaaaaggaccaccagggttacaatcaccaccaacatgctcttgccagagcgggccttcccctcaggttccctttcccccacactcaggcagagggtttgggtctgtccaagtgggctcacaggcatggcctggattggactggcttgctttggtccaccctgggctacctttgacctcctgacaagtggccagaggagctcttagaaaatcttcttttatagcctaaatgggtgggcagaggtggaagattgtggagacttttgagcaaaaggcatgttcctaaacagggacagaattgaacctctgtaaatgaaagaagtagaatgctttcacctcaccatattgtccccagggatactttgacctccatagtgaatattactggaggtcaaaataaaactggtagtcgtgaaagttgcacatggtaaaaattaccattgacaaaaaattggtgggagataaatacttttaaaaatatttgatatgcattatttgaccaaaagtgcttaacaagatagaaataataacaccttgggacaacttaaaaggcagaatctatagtcagcaaatcatgatgtgtaagagaagtcaagttatttatataaaacataattgatcttcatacagacattttcagtaaattccaaataaactgaatcaaaaaattgtaatgaacaaattataactggtttactgtgctgtattagtccattttcacactgctgataaagacatacacaagactgggcaatttacaaaagaaagaggtttaatggactcatagttgcacatggctggggaggcctcacaatcatggtggaaggcaaagaagagcaagttacatcttacatggatggaggcaggcaaagaaagagcttgtgcaataaaacttctttttatgaaaccatcaggtctcatgagacttattcactttcacaagaaccgcacgagaaagacctgcccccatgattcaattacctcccaccgggttcctcccacaacacatgggaattcaagatgagattttggtggggacacagccaaaccatatcgttccatccccggcccctcgcaaatatcatgtcctcacatttcaaaaccaatcgtgacttcccaatagtccccaaagtcttaactcatttcagcattaattcaaaagtccacagtccaaagtctcatctgagacaagacaagtcctttctgcctatgggtctgtaaaatcaaaagcaagttagttacttcctagatacaatgggtgtataggtattgggtaaatacagccattccaaatcggagaaattggccaaaacaaaggggctacaggccccctgcaagtctgaaatccagtggggcagtcaaatcttaaagttccaaaaaaatgatctcctttgactccatgtctcacatccaggttatgctgatgcaagaggtggttcccatggtcttgagcagctctgcccctgtggctttgcagggtacagcctccctcccagctgctttcatgggctggcattgagtgtctatggcttttccaggtgcatggtgcaagctatcagtggatctactattctggggctggaggacagtggccttcttttcacagctccactaggcagtgccccagtagggactctgtgtgggggctccaaacccatatttcccttctgcactgccctagcagaggttctctatgaggaccctgcccctgtggcaaacttcttcctagagatccaggcatttccatatatcctctgaaatctaggcggaggttcccaaacctcaattcttccttctgtgcgctggaagctcaacatcacgtggaagctgccaagacttggcacttgcaccttctagagccacagcccgatttataccttggacccttttagctatggctagagcagctggatgcagagcaccaagtccctaggctgcacacaacagggggaccctggggcctgcctacaaaaccattttatcctcccaagcctctgggcctgtgatggaagggtctgctgccaaggtctgtgacatgccctggagacgtttaccccattgtcttggtgattaacattcaactcctcattacttatgcaaattcctgcagcctgcttgaatttctcctcagaaaatgagcttttcttttctatcatattgtgaggctgcaaattttccaaaattttatgctctattttccttttaaaactgaatgccttaaacagcacccaagtcacctcttgaatgctttgctgcttagaaatttcttctgccagataccctaaaccatctctctcaagttcaaagttccacaaatctctagggtgggggaaaaatgctgccagtctctttgctaaaacataacaagagtaacctttgctccagttcccaacaagttcctcatctccatctgagaccacctcagcctagatttcattgtccatatcattatcagcattttgggcaaagccatttaataaggctctagggagttccaaactttcccacattttcctgccttcttctgagccctccaaactgttccaacctctgtccgttacccagttccaaagtcacttccacatttttgggtatcttttcagcagtgcaccactctactgatactgattacggtattagtctgttttcacgatgctggtaaagacatacatgagaatgggcaatttacaaaagaaagaggtttgatggacttacagttccacatggctggggaggcctcacaatcatggtgcaatgcaaggaagagcaagtcacatctttcgtggatggcggtaggcaaagagagagcttgtgcaagggaactccaccttataataccatcagatctcatgagacttattcactgtcacaagaacagcatgggaaagacctgcccccatgatccaattacctcctaccaggtccctcccataacacatgggaatttaagatgagatttgggtggggacacaggaaaccatatcatgtgctatatttaaaaagtattaaataattaaaagtactattgaagtttactctcaaagttgctcctcaacttttaaaatattggtataattctgacgtcaacgtttatgaagcagagatttaaaatattatgagggacggaagttctatgaggtaaagtggctgggtcagaatagcagggtcaatggaagggcagcattggcaaggtctgactgacttctgtcattttaaatgtgacacagagatgactatgtaagttatcctgtgatcgaatttgtaagcagctgtattaatgaggttacattgaataaacaatttcagtggcataacactgtattttgcacccccctggcatggctagatgggtgcccaggttggagaggcagctttcctccctgtggtcatccaggaacccagagtgtttccttcttgtgccttcaccattcctgtgaatggtgatcatgcaaattgggtcattcttgtcatacccagctaaaacagagtccagagaccagggggaaaaagcactcggggaatataacattgttttgagaatgtaattctcggcaagcccagctgctgaaactgcctgctgtaacctgaaaccagttttctattagtttctacaatgacctgccaggaagctaagactagttttacccaccatcaccactcaccaattagaggatgccaattccctaaaattttacttatgccaatgaccttattttcaaaacaaaacataacatagctctttttataaaacctccaaccttctctttgttcttggacattctgaagagcacttggactatgtgtattccctgaactgcaatttgtgctttcccaaataaaacatttaaatttacccattcaccaatatattttattttgactttgatatctggggcctcatccctacttgcatgcatcagcagaagaagaaagagcacgaagaaatgtccacaggaggattttatgggcctggactggcagtggctcacatcgatcccactcctcttccattggcaagaactggccatgtgaccacgcataacacaagagagcctgggaaatgtagtccaactgtgtgtgcggagaaggggagaatggattttgatggacgactagagttctctgcaacagtaaacaacgtgtttggatttattgtgtttctccaatttttatggagtgtctgctgtattctgagcactgtgctaagagctggacagagagctaatttagcagcaccttgcgctagaagaagctctcagtatataaagggagataagacatggtatggagtgcctgctatcctaagagacagaaggataaagtgaaagtcctagaagtcccatgaattattctagaaggggaagctgcctgcagaagatggtgattgaggtccacttttagaaataaagcagatttggttggtggagggagacagcagcagaagctatggaagcatgtaatttgtgtacaagatgtattaaggaacactatgcagcaattaaatcagattttcaaaaacaattgaatagcatggattagggctcatatcaagtgagaaaagtaactggggatgtaagttgatcccatgtttggaaaaactgtgtgtctaagattagatgaaagtatacagacatgttgggtaatagttcttaaattttataaaatatactgtttaagtaatcagaaggattccatcaatgggctttggtttgatatactattttctgtaagccagatatacatagttgtgatgttggccagtcattctcaacatttcctaacactgctgccccttttccccgaccccctaccctgctatgagacggtgacctggtctctattttctgccctggaatctatgtgctcaataaatactccctgaaataccacacttggaaagagacctcctttttacaatgtgaaatgaagaaacaatgacttcctctcgaagttatcttccaaacattgattcttacaaaattgggatatgatctaactgcaagcccatagcaatgtctaaatttgaatgttgtcttttgctgacgccaaatgcagagccctgcagcatttgtcatgcagggctctcatgtgaccaccagagggcagtgcatgactgctatgacattgtttacttcaaaatgtttgcaagtgagccttcttgagaaaataaatgttttataaacttcgagaatttgtttttttctttaaaataaggaattatttctttaacagcaaataaagtttagaaaatttagttctgtaccttagttcaaactatctgtattttacagtctataaggctcattgtcagcaccacaaagctgtgtaaccaatcttcttacccatcctgtgtgacaggttttgtttgttcttttttaaaggtaaaagtctaagaaaatgcttcataaatcctactttcagaccttgaagtggaatacttctagttacactatgtaaaaacttgggttgtaccttgttattttcattgaacagatagcaaaagaaaaaaaaaggagaaagtaaaaatgaaaagaactgttctacatcctgaatttaggctgtcagtctatatattgaatttataacactgaagcatcagaagcaacctaataatgtcctgctccgtccttcctttccatttataatttttaaaaaggcaaccaaaaaaatgtttcattcattcttcaaaataagttgcaaagaaggatcgcaacagtgaatgtcctcattcacacttccaataacatctcaatgttgctcaccataatctgccatgaaccgattcaggagccacccagagctttattcacggcttccgaatgtaccatgtttcctataccagaagcctgcccaatgtcagcgtctctctgttcagaggccataacaacttttgtcacggctggcctggatttaaagcaacattccagattccagaactagttttattgtctcagctgttttcttcctcttcaggatttatcaattttagaaaaaagggtgtatatttgtcttgcctctttctttctttctttctttctttctttctttctttctttctttctttctttctttctttctttctcttgcctctttctttctttgtctcacctctttctctttctttctttctttctttccttccttccttccttccctccctcccgttcttccttccctctctctttctttccctcttttccttccttccttctttctttctttcctttctttcttctttctttctcctttctttctttctttctttcttttctttctctcgcctctttctttctttgtcttgcctctttctctttctttctttctttccttccttccttccctccctccctcccttccttccctctctcgttctttccctcttttccttccttcctttctttctttcctcttactctctttctttctttccttccttccttccttccttccctccctccctccctcccttccttccttccctctctctttctttccctctttccctcttttccttccttccttctttctttctttctttctttctttctttctttctttctttccttccttccttccttccttccttccttccttccttccttccttccttcctttctttctatctttctttctttctttctttctttctttctttctttctttctttcctctttcctctttctttcttccttccttccttccgtccttccttccttccttccttccttccttctttctttgctcactacaacctctgcctcctgggctcaagaggtcttcccacctcagcctcccgagtagctgggaccacaagcacatgccacccatcggctaatttttgtatttttagtagagacagggtttcgccatgttgcccaggctggtctcaagttcctgggcccaggcaatccgcccgcctcggcctctcaaagtgctgtgattacaggcatgagccagagcacctggccttgtcttgccgttgtcaaagaatctgtgcatatcacattttaagaccacatttcccaagctagaactttcagtaagcttgttgttcaaagtacaaaattcttatctgttttaaaaaaatttaattgaagattagtacctctgctttcctctgacagaaggacaggattcattataccatggttgaaaactaaacttcatgtatacgaatggctagagaggtctgaagttttatagagcccttaacatcactcaagcgcacttggcattggccagaccacagtgggaaacatgtcagcaaattgaggaaaagagactttcccaagtacacgatggtcatccaggctccatgtcactcacataaaaatctgagcaacatgtaatcccatccccaccctcacaccaaagccttgcacctcacagaatatcttaatgttccccattactaatgacagctgctcccgtttctgctgctgtggcagggagtcttggcactgtcttagcagggcttggaatatgatcagttactgtgtttgaatgcacaagtctgactccttcatcccaagtgtcattatttggagaaggacacggagtgtaacttcttctgtgcttttcacccaccatagctaagtaccatgtttcttcaaagagtgaatctcttgggccccggggaacagagttgaccaagtgatgggtgtcctctctgagcttcatgttttcaacaaagacatctggtaccacacaagttcttgagctacaccttgatttaagatcccataagcttgctgttattttatcaacagcagaccatggtgtggcatctggtggaactttaagagagtacagggtggcggctgaaccagaaccgtacgagaacactccagtctcttccctgatcattgctgaggtgagtactgcgctgggacagttgcggggaaccatatactgacgatgtgtacgtatttccattttcactggatacagcaaagatgcctttgttttctgatgggagagttcggaactatccttcataaatgccatctccacatctgtatcaaaatagatgtcttctaatttaacattcccaaaggcttccaggacattctaaacactatttttatctctgttctgatcattaaggaagtcattccgcaacatccgagctagagataaactcaccagtttacaatatggtgagtgaaggatcaagaagccaaaattattcaaggtaaaatctttatcatttccgtctttctgccactgggcacggatcgttttgcagtcgacagaatagcagtgatctaatgcactgaagcagcactgtgtggagggttttccatctactataggacattcagagagcatatcaggctcataaaagccataggcacgttgcatatgtgtcccacgaagccctcgttcaaaaattaaagttgcatttggctcaattagcagagctactgctctgactctacctgtcagtctaacttttcctgtgggatatacagcaatatctcctacaaactccagggcaaactgtccatcccaggagctggactcaatccagttaacagcactgaagacagcagctggggctccatagcatgcatgagttgtgtcggttccttctatatctatatccccagactcctcaaacagctgcatcaaattagtcttcactgactttgatttgtccttgattgtctctgttccaacttccagtcggccagtgcaatcgtaggaaagattttttctctccataagattctgaaccacaggcatgcaaagagagttgatctcttctccatgtgtgcaggagcccatcttggcctggcccaagccaatggtatactttccagcatctatatcaacatacttttccaactttgaaaatatttccttggcatgcctcaggtctgatatccattcaccgtgtccaatcccaggctagtcttggacctcctgacttcgtgttcggtttggtgccggaactggatctggtggctctgttgccacctctgctgccctcccagcaccgctcagttgcacgccctgcaagcccaggccatgggcccaatcctagagaatttgtaaagaaatatttatttgcttttatttttcaatttaaatgggaagccagtgaaaattccacacaagaaacaaatataagacaatccagttactgggagatacaccttgtgatgcgtgcaaacttgtaaaattatttgtccatcctcaggatttgcatccagattcatctctccttcctccatctataaacatctgtctatataaaatatcttttgacgattagatgtaatgcactaaagatatctggtaatcatgttacttctttgtgataggaatcttttggtagcaagtgatctgcttatgggatggtgagaatataatggaaacaagggaggacagaatacaggcgatcagcaggaggtctcagccacaagtgtgagaaagtggacatctcatggttagcatagccaaagtctagtatacattgtctgctatccccaaagaactcagtgtctgtgtgcgaactttgcaaaaatccttccttctttccttcctaccttccttccttcctccctccttttctctttctttctttctttctttctttctttctttctttctttctttctttctttctttctttctttctttcttctttccttccttctttctttccttctttcctaccttccttccttcctccttccctccctccttttctttctttctttgtttttctttcttttctttctttccttccttccttcttcctttcttttcttcctttcttcctttcctttcctttccttctctctttctctctttctttcctttctttcttgacagagtctcactctgttgcccaggctggagtgcagtggcacgatcccagctcactgcaacctctgccttgtgggttcaagtgattctcctgcctcagcctcccgagtagcttgtattacaagcatgtgccaccatgccaggctaatttttgcatttttagtagagacggggttttaccatgttggccaggctggtcgtgaactcttaacctcaggtgatccacctgcctcagcctccccaaaaatcctttatttcttaaaagaggatcagatcagagtaatgaccagtggttataataattttgagggagagattaggttttattacaataacctggtagctgtgctttctaccatacttgctataatccttagccccctgcacaggacagtgggcgacctgaaagcacagtctacaccttggtcccgactatgtcgcagatttttagcccagcacctggccgagcagaggtgccatgtacagctgaatgaaggcactttggttgcaacatctgacaaaaatactacggtatttgaaattaggactttcctagacagcccagatatgaagtcatcataaacacgcatcttgacaacctaccagatcctttaatcccgactgccttcccactgcaaccaccagcctggacctacttggtcccaggtatttcccagaatcacgctcttgttttgccttctctgcttacattggcctccttcattggcctactctgtgttacttcactctgggtcttgggggtcacctctgacctcccttggatctcccttttctggatctcccttctctggggagcccattggatctcccttctctggctcccctgccatcctcttgtttctggttctgcacttgacttggattgcagcattatcccaactccagttggaagacgcccccagcatgctgactctgggagccctcactagccaaagctaccccctcacctcacctcttctggcatcccctcttggcatgggagttcagatgtctacaaatgtgtaagtaagaaaagtggtttcatgccttcttactagaaaataattcccaacttctgaaaaattagtagaaaattgtgggtttatttgtcttgcttcttggaataagaaaacttgatgacataaaatagagactgcaatattctgataccggtaacaaagagtaatttaaatactgcggccagatatattcccttcttttatgctcatgacactgtcatttaaaaacatggatcttaaggtaaaatttactctgctatctaaggatttctagaataagaaagttttttgcttttttgtaaaaattccttttaacacaaatggaactttagaaattaactactcacattgttgcttttcataaatgaggcagccagaacccagagaaatggtgtgtcttgcccagtgtcaccaatctctgccttgagagagtggacagggtcttactccacttactatctccagattccaggcccgtgcttttaaaatagtaggagctcaaaaaatctaaacagaactgaaccatattgagcaatttgatactgtcttgagattttcttactactggttgtaggttcacctttgtttctggtaaatcatgttaaaataacatttcttgagcagagtaacttgaaataaacaagcctccttttctgacactgagcagggaggattatgctgacaattctgactacctttctatatcctctctgagaagcagcactctaaaaccatctgatttgggctcagagcagcccagattgtaatattatttgccagttaattgggggcaggaagccagctctgctgccccccacacacaccagcagagacttcagaagaccttcaaccactgcaggaatggctctgatggtctctgaggctctctttcctcttggtcagctccaggtatgagatttatacacgttggagaggttcatgttgctgttagaaggtaggtctttcagaatattagattttgtggaactaaagtttaacaacaggattttacttataaaacaataactatgtgaatgcataaagcttggtgtggctataaatcagtggtgttttattcattaaacaatttatttaatgataagatggcctaaatatatatttttcagagaagtgagcatgcctggtgatttacaatgcacagaaagacctgatcttctggaaaaggaacagaaataaattatgtcaatatatcatgaaatgcagaattgcagaattctaaaactgctcttatctacttttgctgcagtaactgaaaaaatcatggagttaatatggatctttcacaattttttgacaaataaaaattgtatatatttaatatgtgaagtctgatgctttgatatatatatagtgaaatgattatcataattaagctgattaacatatccatcgccttatatagttacttggtgtgtgtgtgtgtgtgtgcgtgcgtgtgtgtgtgttaacatttaacattattactctctcagcaaatgtgaagtactattaagcatagttaccataactatacagtattagtattattaagtatagttactgtactgtgtatttgatccccaaaacttattcatcttacacttgaaagtttttaccctttggctaaatcccccaatttcccattcctcagcccctcgcaacctcccttctactctctgtttctttgagttcaacgtctttggattccatgtatcagtgagagtatgcagtatttgtctttctggtcttatttaatttagcataatgtcttttatgttcatccacgctgccacaaatagctggatttcctttttttaataagactgaataacattatgtatatatctcatcaatggtatagcatgtgtcagtacctcattactttttaaatgctgaataatatttcatttatagatatactacgtttgtttatttgttcatcggtaggtagatatttggattgattctggggtttttggctatcatgaataatacctctttgaacatctatgtaaaagttttgacatatacgttagcttttatttcttttgggtatttctaggggtagaattgccagcatcagcccacccagatgcatcccataaggattccatattttatgcctttgccaatcttgaagtcacgctttctcgcgcgaaaagaaaaggtgacactttctctcctcctttacactntcacactctnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnaattaaaaaaggaaattatgaaaacaattccacttataatagcatcaaacataataaaatacttaggaattaatttaaccaagaagatgacagactagtataatgaaaactataaaatactgctgaaagaaattaatgaagacatgaataaatggaaccacatcccatgttcacagattggaggacttaatattgttaacatgttaataccacacaaggtgatctatagatcaatgtattccctataaaagttccaatgacattgtttgcagaaataaaaaaaccctcattttaaaatttatatgcaatctcaagagacctggaatagcaaaaacaatcttgaaaaataacaaagctggaagactgacactttcttattccaaaacttactgcaaagccacagtaatcaaaacagtgttgtactgtcttaaagacagacatagagaccaatagaataaactagagagatcagaaataaatcctcacatacatggtaaaatcatttatgacaagggtgccaagaccattctatggagaaaggacagtcttttcaacaaatggtggtgggaaaattatacatccatatgcaaaagaatgaagctgaacccttacctaacaccatattcaaaaattacctcaaaatgtaacaaagacctaaatgtaagacatgaaactagaaaactcttagacaaaaacataagcaaatgattcagaacattggatttggtagtgatttttaaatatgacactaaagacacaagaaattaaaaaagaccataaaaaatttttaaaaatgtgcaccaatggatgacctttcgacttaattaaaaaatttgaaagcacaaggaattactttttaattgccatgatttaaaagtagacatatttaagttttaactttgcatttaattttatatgatgaaatttataggtacatgagagaagagagaaagtgttatcttttttgcataagaatgtaatttcagaaatgtaatgatgcaaatttttgaatccttatgagatgtatctggatgggctaaagtaggtgtggaatggctctcctgggagctctgtcacaaaggaagtctcttgtcgaaaattcaacttttacattgacagggctctactgttttggaacacaatttgaccattctttaaaatgttaagcatactattaccatataacacctctgtattataattgccctacatctaagggcaatcaaactgaaaaatttattccaatgaaagaaaaataacatttggaggcttaatgaaaatttttttggtactaatgcaaaatcagacctagtacttctaggtgtatagaatttaattgccctgggatatacgacaatttaactgccatggatatagggcaattataacacaagggtgttaattgttaattgccctatatctaagggcaattccacccttagacatacacaagaaaaataaaaacagatgttcatatcaaaacttagacataaatgttcaaagtagcattatttataatcaccaaaaagtgaaaataatggaaatgtccatcaacacatgtgtgaataaatagaatgtgccaatattcatacaatggaatactactcagaaacaaaaaggaagtgctgatccatgttataatattgacaaaccttgaaaacattatgccaagtgaaataaatcaatcacaaagactatacactgtataattccattatggaatatctccagagaaggtaaatctagagagtcagaaaataaattattgtttgcctagggccaggagtaagaggttgggaagtaactgataataggtatgaggattcttttggggttgataaaaatgtttcagaactgattgtggtgttggttgcataactgtgaatgcactaaagccaatgaatagcacaatttaaactgggaattacatagaatgtaaattatatttcaataaagttgttaaaatgggggatctggcattctgattttttaaaaaatatctattttggagataatatttagttgttagttggtgaaattatacacctagatgtactaggtctgctttcactttagtagcaaaaaataatttgcattaagcctccaaatgcttttttttgtcttttattagaataaagaagttaaaaatctaccttctgtaaaattaaatccattaaattgcttcaggagtcagagttatttgaaaaaatatttttatatttataatataataagagaagagggtcttgctatgttgcgcaggctgatcttgaactcctaggctcaagcactcagcctcccaaagtgcagggagttcaggcatgagccattatgtctggcctgatgcttatatttttgattgaggaatagaaacttagtgttaggtgaattagtgcctgcaaggcttgacaagacgcaaagtcaatctgaaggcagcgtgagctacccaccacaagctcttgtgtatcacgtgccccttagagaaaaatcctaaaactgctaccaagaaaacttacagatgtacccccacccatgaaatataatcacaaaagtttaatcgtaaattgcaagccaattttattcttaactttatgagacaaaagaaacaaacaaacaaacaaacaaaaaaacttctatcagcccgacctggggctctaggatagaattaagccctaatagatgaaggcatctatggtctagaatggttctagttttctactattttacggagagccaagactcaaattattttctgcatttggtgatgcagataactttggttaagaaaggtactaatttgtttgccttttgagatatgatcctaattttctgaatacagctgttttttcacatgtcatgcaataaacagtattttcacagggcacacagctaaaaaaagaacacagtcttctcagttttaaacccaagaattagaaactgtgaaaggccactattaactccatttttaaagagactctagcaaaattggataatagcaattttcacaattatcatacaatccttttggtgatttgttgatatcttttatttctgttcttccagaagatcagatctttagagcatcatcagagaaaaggtcaaattcaccaacatgctcatttttcagaaaaacatatttgtttgagaccatcttactttaaaattaagtgtttaacaaaaacaacaccattaagttacagccatatcagtgtggtttttttgctccatataattattgttttgtaaatgaagtcctgtcattaagctttagttccacggaaactaatattctggaagacctaccttttaacagcactgggaacctcttcaacgttcataaatctcatacttggagctgaccaagaggaaagagagccgtagaaagcgcctgggttcagccattctggcgatggataaaggtcttctgaagtctctgctggtgtgtgtgttgggcagcagagctggcttcctgcctccaattaactggtaaataatattacagtctgggctgttctgagcccagctaagatggttttagagtgctgtttctcagagaggatacagaaagctaatcagaattgtcagcataattcctccctgcttagtgtcaggaaaggagccttgtttatttcaaattactgtgctcaataaatattattttgccatgggtcaccagaaacaaaggtgaacctacaacgagtagtaagaaaatctcaagacattgttaacagtgttaaataaagttcagttcttctaaattagattaattgagcacctactactgtatataaaaagcaccagcttggaatctgaagatagcaagaagaataagataatgtccattttgcaagataggtgaagggattggtgacattaggcaagacacaacctttctttgggccttagctgcctcacctgtgaaaggtgaggatttgaccacttgctttctaaggttcctttcaggttagaaagcattttgaaaagcaaacaaggctgggcatggtggctcacgcttgtaatctcagaactttgggaggctgaagcaggcagatcacctgaggtcaggggtttgaggcaagcctggtcaacatggtgaaaccccgtctctattaaaaatacaaaaattagctgggcatggtagtgcatgcctgtaatcccagctattccagagcctgaagcaggagaatcacgtgagccaaggttgtgccactgcaccccagcctgggcaacaagagcgagactccgtctcaaaaaaaaagaaaaaaaaagtaaacaacagaatgtccttattccagccatccctaaaaagcagagttagctttaccttaaattccatattttaagtgatagtcacacacataaaagagaagcatctatatggtcatattgttgaaatgattctttgttgtttgtatcagaattttgcagattatattttttgctgccaacttttcttattccaccaagcaggataagccacatttttttttttttttttactgtgaatacactttatttagtcatttttgtttagagttgaaactctgggaattcaacatttatatccttgcctgtgagcttcatgtagacaccagaaaaagtttcaacgttgcgttccacattgttttgctgcgctttgtccaaatgagcctttatgagacggctgccatccagtttcacgtggattctcttgcccacaatttcacttgggaagaccaagtcctcggggattgagtcatgcatggctttcagggtacggctcctgggatgcttttgcttattttttgtacagctttttcgggttggcttaggcagaattctcctctgagcaataaagacaacatgcttcccccaaactttttctccaattcacgtactagccagacttggattttctggaaagatttcagttgaggaaagggaacaaaaattatgatagctttcccaccaccaccatcttcagtttccttggctgctgtaatattcagctccctgaactgaaccttgaggtgtgagttaatctccagctccagaagagcctgggagataccggacttgaacttgtctggcttctcgctcttgggcttcatgatcctggtgctcgactaaacatggccttctccttgctgagtgccaccttaggaaaagtgaaaccacgttttaaaaccaatttgtcaggaattcataatcattttctagctaagatagggtgagaccccttttcttccctatacatttttagatatctaaaaccctgtcccatgtggggatgatggggtgtacaggggctaccttggttagtgtgggtcccccagagttagcatgctgggaaggtttgttacccggagttgcaggtgactgcagtcaaggctgtgcacagaaccaagagcagatgatggcagattggccagaaaagggagggccaatgggttaccaaaagcattatcaggctgggtgcggtggctcacacctgtaatcccagcactttgggaggccgaggcagtggatcacctgaggtcaggagttcaagaccagcctggccaatatggtgaaaccccgtctctactaataatacaaaaattaaccggacatggtggcacatgcctgtaatcccaactactcgggaggctgaggcaggagagttactggaacctgggagtcggaggttgcagtgagctgagatcacaccactgtactccagcctgggcgacagtgcaagactccgtccccccaccaaaaaaagctcaatatcagaagtgtcccctgagacttagagtgaataacacatattaggacaatcatggagactaagtaaacagagaaggcaaagcaggaggctgattctggaaaatacctaggaccaggtaggtccgggctgggagatgctgtgggaatataatcaggtctaaagggctggcaggagagacaggtgtgtgagcttggaggactgagacttgggggacagaagaatgtggtggaggaccgtgtgtcatccttgaacaaagacaaagaccacctgttcgagcctcgagagcttcatttggaagctgaggcagagtcattgacctgactgtagtgatgtggcctcatgagtgaactggaccccagtaccagtgctggcagtaaaggaacaagaaaatgtcctaaggctcccatcaatgactccggatctcatgggtgttgctcctttgccctgctaggacctgagagagctgatgccttgaagtcattcaacaaaggtgttctgctcatggtttttctcccttaggtgcagggctagcactggccaccagggaggctgtgcccacagtgctctgctggagcaactggagccagggaggagccacccaggggtcattctggccattttcttgccaccaaccagacctcacttcctgttgtgagcattcatctccacagtgtgagctggtcagatggaggaaagttgggacaaggggcttgagatcctctcatttactcattcagtaactaggtcccaagcacttatcttgttccagggctaagtatggtctgggtctctcagtgaatgaatgacatggcccttgtcctccaggagctcacagtctggaggggaggaggagggacggaatcattaaaatagaggaaaagtgcgagcataatattgaaatagagcacagtgctacgaaggggagatgaagatcacttcaagagtgggaggtaggtctgaaaaggcctcactgaggtgaaatttatactgagaaggataattgggaccggccctgtgaaagtccgagaccagtgtgccaggggaaggatcaatggccaaaaagtccaggaaaaagtgagtttggccagagaggaggtagaaagagggccagcgtggctggagagagcttgcaagtagaggaggggagggaggctaaaggcaggggtggattctggatggccctgcattagtaatctacagctgtatgaaagcattatcccaatcttagaggcttgcaacaaacacgtagatcttcctgtttctggggcccaggaatccagcaggacttatctgggtgcttctgccatggtatctctcaggaggctggggctgtggtctcaactgaggctcgagtggggctggatccaatgctaagcccactcatatagttactggaaggattcagttaggactgatggcctgagctcccctctgtccatttccctctctatatcctaggacacatgagcctctccatagggcagctcataacacagcagctgattccccagagtcagggatctgagagagggagagacagagagaggacaccacagagaaagaaaaggagaacagaggagagaaagagcaagatactagtcatggacattttataactgaatcttggaagtgacatctcatcccttctgctatattctattggttagaagtgagtcactaagtcagtccatactccaggggaggacatgacacaaggatgtgtacaccaggaggtgggggaacctcttggagccattgaggtggctcctaccacataggctgagacgtggcactcgggtctgaatgtaaacaaacatttaaccgtcacaagcgctgatgaggatgacaatatcagaatttactaaatttcagccctctaatagtacacacagggaaatgaggccaggaggaatgggacatttcaaagatcacatggcaggaaaccaggcctctttttagttgtgttccatgataattcctaccatcccttcatgcatttcatgtctaatttgtccattacctacatgtgataccagcccacaggtttcctaagtgttatctcacacatgcctgatgacatccctagggagtcgatgccaggatgatctccattgtgcaggagaggaggctcagggagaccctgagaggcacagtggctttgcccagagtcacataagtggtaagaaggaggagctgggtctgaacctacatctgtctattccaaggtgggaccctggctgcacccaggccttccacagggcctgtagggaggcagggttagtgaaattctgtacatctatggagggtcattgggtaggggggtgtgcagacagtagcccacagggtcatttgcagtcttctaatcagggaggggactcagggaggggatgcaggtaagggaaccccaggatgtacagaaggtgtggagagcatcgtgctgagaaatctggctgcaacctcaacagtgatgtccagccaggtctcaggggtcgggctgctcccctccccacttcagagtaaacatcaacatgactgacattccacgtgccttccaatgacaaaaccttccctctgcctttcagagatgcccagtcaatgtcccactcccaggacgtcttcctagactgcagtctaccacataattgggttgagtttatcaatgctgtgtcccctccccagcttctggcagctctgctctgtgtggtccagccctatcccctccctacacgctcacctgcacctacctccaggcctcctcaccctcagaccctgcctcagtggactctgggtgctccagctgggccaggagtaggtggacacggtgctgcaggtccgggcaggcatgttgagctgcagataggccccagcacctcgagagggggcaactgtgggggctgatagaaatcactggcgtaatgatccatccaggtgcccagcatggaggagatggtgcagggggaggcaggtgagcagaacacatacaggaccagtctctccttccaccctgccctccaagggacatccctgggaaaacctctaacttggctcatgcctaaattcctgtgtgcagcctataggtctcctcttcccaccgacacttctcttaccccacccccatggtctccatctgtgatgtcagcgtgcgtgtgcgtgtgcgtgtgtgtgtatgtgtggatgctgagagctaacagttccacaggggatggtcaggcaggatggtgaatcaaggtccagttgggactgtcttcctggggaagacacaggtggaccctttcccgagcagctgtgcctactgtcaatgctcacacacacggggcccacgtgggatgcagacagggcaaacacgggagaccccctgcacagaacatgctcaaggcaccaatggttcctccaacatctgttcccacctgtcctgtcccctgcagccagaaagtggtattagcctcacttaaccatatggaattgcagggagtggctagttccctaaatggtaatgtaggcactataacaattagaagtgaacatggatgtgggccaggaaaaagctccggatcttcagaattacacaaagcacccacacccctgcctcttgtgtagaggaaataagttccagagaaaaaatcgtctggttcaagttcacacacagattcaatgaaaagctggtatcttggttaggattagttttgcctacacatagcagaaaacccagaataacagcaccacagagaagagagagaaagagttatttctttctccaataaaggagctctggaggtgggctccctgctgctcctggatggccccccaaggcagtcagccagcatgaatctttctgccccagcatcttcatagctggtttccatcctgaaggctgtctcagggcccacggtgactgcttttcagagggaaaggaggaggaaagaaggacaaaggggccgcacagctgtgtcagttccacctaagcaccctccccaaagaccctctcagtccttctgctcacagctcatcagcttgaactttgtctggtccctcttagctgcaggagaagttaggaataatagccttttagtttgtgtggcaaattgtgttcctaataaaactgagtctctgtttcgaagaaagagacaagaatgaaaaccgcgtgtacacgaacaaagctctctccctaccctaaactccaagctgaaggaggcaggagggattgacttcttttcggctgaattgggcattccttcacctaccacggcctctcttttcttggtgattattcattctcggagaggggacaaaagccgtgattccaaactaagacattggagtgggaaacccgctttcagttcctcagcagaatgatgagaacctctgtgtcgtctttcatctgtctgcgcattggcagaaggtctgagaaaaccctcttttatatcctaaatggaagagcggaggtggaggattgtggagaattttgggcaagcagcatgttcctaaaccgggtcacaattgaagctctgtaaatgaacgaagtggaatgctttgacctcaccatggcccccggaacaccttgacctctattgtgagtattacagcaaaaacagtccacacaaaatacaactaataatggttgaaaactccacatggtaaaatttatcactgacaaagaactgatgaaagaaaaaaatacttaaaaaacattttgacatgtattatttgaccccgggcacatgaaaaggtagaaagaatgattccaaaaaaacttaaaaagagagaatatatagtcagtaaattatgtggacaaatgacttacataaaacttaattgatcctgataacaatttcagttttctaaagtataaaaaatcaaattttagtaaattaaaaataaaagtgagtaaacgactttggtttactttctgtatgttaaaaaatcagtgagttaaaatatcttactgaagtttgctgtcaacaaaattttaaactgttgataataattctttttttttttttttttttttgagatggagttttgctcttgttgcccaggatggagtgcaacggcacgatcttggctcactgcaacctccacctcccgggtttaagcaattctcctgcctcagcctcccgagtagctgggattacaggcatgcgccaccacgcccggctaattttgtatttttagcagagacaggatttcaccatgttggtcaggctggtcttgaactcctggcctcaagtgatctgcccaccttagcctccccaagtgctgggattacaggcataagccaccacacccggcctgtgttgacaataattttgatttcaaaggttactgagcacagaataaaaatattttgagggatcgaatctctctaaggtagtggcagggtcagaatagcagagtcaaatggaaggcagcacttgcaaggccaaatggacttttgtcattttaaatgtgacacagagataataactatgtaagttaatcccacaatgaaatttttaagcaggtttattagtgagattacggtaatagccaaataaccgttcatattccaggggtgtaacactgtatttcactgccccctggctttgctggttgtgcactcagtttggggaggcagttctccctgtggtcactgaggaaccctgaattcttccacctggtgccttcaccatcctagggacttcaaccccatctgcattcagcagaagaaagagaagaagcatggagaaatacccgtgggggattttatgggcctggactggcagtggctcccataagtcccactccccttcccttggcaagaactggtcacgtgactacacataacacaagggagtctgggaaatgtggtacagctgtgtgtgcagagaaggggagaatggattttgctggacagctagagttctctgcaactgtgagtaactcatttggattaattgtgtttctcattttattttgttttaagacagggtcttgctctattacccaggccgaagtatagtgatgtaatcatggctaactgcagcctggaccttccaggattaaatgatcttcccaccttagcctcctgagtagctgggaccacaggcacgtgctaccacacccagataatttcttaactttttgtagagataggatttcaccatgttgcccaggatggcccccagctcctgggctcaagcaatccctccaccaactcgacctcccagagttctgggattacaagcacgagccaccatgcctgaccaaacgtgattctcaaatgtttgttgagtgtctactgtatgctaggccctgagctaacagctggacagagagctgcaccatagcctgagagaagctcccagtctgcagaaggagatcagacgtggtatgaagtgcttgctatcctaagaaggataaagtgaaagtgctagaaggctagtgaattattccagcataggaagctgcctgaagatgacagcaactgagctccatttttgtagagaaactagatttggctggtagagggagacagcagggggagctacagaagcatgtaatttttttttttcttttttgagatggagttttgctcttcttgcccaggctggagtgcaatggcgctatctcggctcaccgcaacctccgcctcccgggttcaagtgattctcctgcctcagcctcccgagtagctgggattacaggcacgggccaccacgcctggctaattttgtatttttagtagagagggggtttcttcatgttggtcaggctggtctcgaacctacctcaggtgatccgcccgccttggccttccaaagtgctgagattacaggcgtgagccaccgtgcctagcccagaagcatgtaatttatgtacaagttatggtaaggaacgctagacggcaattaaagtcagattttcaaaaacattaaatggcacgaataagtgctcatactaagtgcaaaagtaactgggaatacaggatctcccactgtttttgaaaaaaatctctatatctaaattagatgaaagtatacaactcttttgagtgatagttttcatttctctgaaatgttttattgtttataaaatcagccgggcatggtggctcacgcctataatcccagctactcgggaggctgaagcaggagaacagcttgaacccaggcagcgaaagttgcggtgagctgagatcgtgccactgcactccagtctgggcagcagagtgaggctccgtctcaaaataaataaataaataacataaacatcagaaggattccatcaatatactttgattggtttactattatctttaaaccagacgtacatagctttgatgtaaaccagttattctcaacactgtcccacccctcaccctgctaacgctgtgagactgcaaccttgtctccattttctgtcctagaatccgaagcctcattcaactgctcaattaatactccctgaaatggtgtactggggagagacttattttttcataacgtgcaatgacaaaacaaagacttccactaggaagcacccccaaagcactgaatcttacacagtttgtttgcgatctaatcaaaatcctgagaagacaccttttatttttatttatttattaaaaaaaaaaaaaaacaacacaccgcacttttaatgtgatcttttggagacgaagttttggagccctgcagcattcatgccattttctatatggccaccagagggcagtgtgtgtgcgactgcttttgacgatgatattgcccatttcaatagttgtccaagctggccttcttcagaatgtgttcatgaccttagggaattcctctttcttttctgtttttttgatacagggtctcgctctgtcactcaggctggagtgcatggcatgatcatagcctactgtaacctcaaactcctgggctcgagcaactttcaccgccccctgcccttagcctccctagtagctaggactacaggtatgcaccaccacacctagctagcttttaaaattttttggagagacagagtcttgctatgttgctcaggctggtcttgaactcctggcctcaagtgattctccagacttagcctcccaaagcactgggattacatacaggtgtgagccactgcacctgactgagaatgtcttcaataaacttttttttatatctctattatcatttggaagctaatgaaaattccctataaggaacaaatataagataatcctctgactggaaatgtaccttgagacaaaggcaagccatggacaattgcgtctaaccccagacctgtgtccagatctgccgctctcttctccatccattcattctacaaacctctgtctctacaaaatatctttcgacaattggacgtaatgtactagagacactttccaatcacagcaaccccttctttgtgatatgaatcttttggtaacaggggtcttcctcataagagagtagaatatgaaataaaggaagaccaaataaaggaaattggcaggaggtctcagtggaagtctgtccagaggggaaatctcatgttagcatagcaaaaatccattatacattgtctgctatctccaacgcagtgagtttccactttccctctaaaagtttgctaaaatcctttatattctaagaacatcacgttataataacaaaagtagtcataacaattttaagaaaaagacaaaattactactttaatcaccagatgctccagcttctacagttcatccctcagtattctatcataatcattggtcagcctcacaagatattaatttgagaggagtaattctacaccttggttctgaccatataccagagtttcttagcccagtacctggccaaggagaggtgccttagaatacccagcaaatgaacttgggacacaagatctgacaggttggagaagagcatttgacattgggactctcatttaaaacagctcagatatgaggtcaccataaacatactacttgataacctgacaggctctttaaacctgattgtccttccacagcaacccccagcttggacctacctggccccaggtatttcccagaatcaccccctgctttgacttctcttgcttactttggtctcccttgtgtcctactgggtggttcttcactctacgtctccgaggacacctctgacacccttggataccctaatgaatctcccatttctggccgacctgccatcacctgcagctggttctacacttgactttgattgcagtatcatccacactccggttagaagacactcccagcatgccgagcccaggagcccacaggagccaaagaaggccccctcccaatctattccaacatccgcattagggataggggattcaaatgtctacagatacgttgggaagaaaagggtctcatgccatcttacttagaaaatgattccctatttccgataagttactacacaattgtgtgatttatttgttctccttcatgcaataagagaacttggtataaacaaaaaaagttcgatgtcctaatacaattaaagaatatcttaacaatgtgaatatttctttcctatttcttttctttcttttgtttatgatcttattaaaaaatgggaatgtgataccctgtctctactaaaaatacaacaaaattagccgggcgtggtggcgagcgcctgcagtcccagctactcgggaggctgaggcaggagaatggcgtgaatccgggaggcagagctcgcagtgagccgaaatcgtgccactgcactccagcctgggcgacagagcaaaactccgtctcaaaaaaatacgggacttaaagtaaagctatatctgctttctagggatggctagaatgggaacgttctgttacttgtttgtttgcctttacaaatatttttagttcagtaggaaccatagaattcagtgggtcacatccttgcattttgcaggtgaggcagataaggcccagagagagggtgtgtcttccctaatgctcatggtcaccaattcctccacctacctgcagaaaataaataggatcttattacacccagtatctccagattataggccagtgcgtttaaatagtaggcatccagtaaatcaagttaattgaattataacttaactcggaggacttgccttccccgccctgtctgcccatgagtgcgtggtgctggctttgaccctaactagatattaaacaggaggagtggtatggggtaaagtttggaacaacacgttttcctttagtgtataaaaattctatgatttccaaatgtatgaaaacgttgattgaaacaacttctctccctgagatcaaagctatctctccctccctctctctctctctctctctctctctctatctatctatctatatatgtgttgcagtgttttatatatgtatcttctcagcccttttgcgtaagggatggggaagagctgtttgtgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnctacatggattctttttttctctgaggaccaggaagaaaaagtgttaataatacctaaaattaccagagagagcatgagctatctgaaaaatgactgaagcacttaggacgaggtgaggaatagtagatcctgaacaatgaggtgacaaaactacagagattaaatattaatacatattaaaatatatataatgtctattatatataatatatagcttatatatgctatataacataatgtcggctgggcacggtggctcacgcctgtaatcccagctctttgggaggctgaggcgggcggatcacgaggtcaggagatcgagaccatcctggctaacacggtgaaacctcgtctctactaaaaatacaaaaaagattagccaggcgaggtggcgggcgcctgtagtcccagctactcgggaggctgaggcaggagaatggcgtgaacctgggaggcggagcttgcagtgagccgagatcgcaccactgcactccagcctggggatagagcaagactccgtcacaaaaaaaaataataataatgtctatattaatatatttaatatgtacatagtaaagaatttaaactcaatagcctcagggtgataggggaccaatgacatgtttctataccggaacccattcaagtttgtatgttaagtcatttactctcttgcttcatgcagaatggagtggagaaacgcaggttgtggacaaaagaatgttgaggaggctgttgatagggatttaaactaggaagggtagagataatggaaacagaaattggaagacattttaaagagatacttaggaaaagaaaagagaaaatggaaagattaactgggtgtgaagtatgagaaggatggaggaatccaagaatatggcaaagctagacggcaatgccttgtttgggaaacccagaaggagggtgttaagagggtgggtaaaatagtaagtatagttttagacacggagaatatcctaggcttgtgatgtctaagtgagatatgtgtgaggtccagagaaacacagaaactgcagacacaactctagcagtctattggtaactgacaaagacaaaagcacacatacaaaaggaaatcacgggctcaaccttaagggcttagcctcaagctcaagctaaagccacatgcctagcttttattatccaaaagcagatgatatgaaaatgatctgtgaatttagagtcaaatgagtctccttagagaaataatccaagttattaaaggcattggccaggagcagtggctcatgcctganatcccagcattttgggaggccgagacaggcgggtcacctgaggtcaggagttcgagatcaacctggccaacatggtgaaaccccgtctctacaaaaaaatgcaaaaatcggccgggtgtggtggcatgtgcctatagtcccagctactcgggaggctgaggtgaaagaatctcttgaacctgggaggcagaggttgcagtaagccaagatggcgccactgtattccagcctgggtgacagagtgagaccctgtctcaaaaaaaaaaaaaaaaaattaaaggatcacctttttactgatatcacgttgtcagcccagtcaaatgtctctccactttctccacttaaatcactgcagttcttatacaaccctctctgcctatccccaaaatattgaatccaacagagtcaacatacttctggatttttctaaaaattgaacagagacagatcaatccctataccctgaaagaaactgcaaggataccatccacacattttatatccccaaagccatttaaccaaaatagcaccaaaaggacagaagatataattggcttatagaagaacattttggttagctgcaaaatgtaacacttcccacagagagctcacataacttaccctctccaaacaggcaagtattggtctataagaaaaagaagccaattatctacaagtatataggttaaaaaatgagtttcaaagattatccctattgttacctcatttcttttattatacttttaaaagataaaccttgtatctcaataatgaagagcatccttacaaaactctccacagagtattcaaatgcatttatttgggatcctgaaatagttctattctagtccttaatgggctcaaacaagctgtacttgtttaaggccaatggaacttttttctttttaaagtaaaaaacctaggacctctcaacaccttttaaccttctagtttctaagaaatttttaatatcaatgcacacttctaaatggaataccacagactcataaaaagtatttcagtaagcatattcaatagtaggaaaaactgaaaacataacaaaaaccaatatattaaagtatattgaagtaagagaattaatagtactgcttttattgagcagtttaaactataatgaaaaattcaaagcctacaatccatccaagaacaagagatgacttacattgcagataatgaaactaaaagtaatgactcagtatattttgtgccaatattagaaagccttcatatattctaaatcttcgagccatacattatataacatagctttccatttcatacattttcataacacacgatcaaccatcatacataatcattatatcagtgttcaaaaacacaaggccctatcgagaccagcctgggtaacatagtgagaccctgtctctccaaaaaaaaaaaaaaaaaattagccaggcatagtggctcacgcctgtggtcccagctattctggaagctgaggtgctgaggtgggaggattgcttgggaccaggagtttgaggctgcagtgagccatgagcacaccactgcgctcccgcttgggtggcagagcatgaccctgtctcaaacacacacacacacaacacagacacacaggcacacacacacatacacacacacacacacggtcctattgtcagataatgacagtctaaccaatgcaaatctccttcagcatgaatcaaaaattgatcaaaactatcaaagtcaaaacatcaccactctcaccatcaattactccgtggtataaacgggtcttccggcataggaattttcttcagctctaccaataaaggaaagctctaaatacacagagcatccctattagggagaaaaggtacctgctggaatcaccatatctatagaaccacagtgctgaaattgacttgaaggaagacgcttcccagcaagattgcattcactcttccaatctgggaagccagggtattaacaagaggcaagaaatgattgccttgtgccctaataggaagctggtggtaaaacggaaaagggaaatagattgttctcagcctgcactcttcccatagtgctgacactagcttactgaaacacattctttcccctcctgcgtacctaaaacaaccatttaaatgacacagaatttcttcctgtatgtgtccactctatcaccaggtactacaacaaagaacccttctgcatgaagtacaaaaaaagactcaactgagtagtgcctaagcctttttaagcataattttgaaacataatataggcttgattttctcaagaaagtattccattcctcctgaaaacaataggactgttttcagtccattgcagctctagggtaacagataataaagcttccagtgggttcctggtaggttggagcctgactggatcacagaatagaggcagtaattttcctatccaatcaacacttccaagtacatctttacaaaggaagaagacataaattcttccctccttctcacccatagtggagcagtaggtatcaaaaggataataaatctctagtgtggttagcatcttattatagcatccagaaataattgttactcattcttagaattttgatgttatgactaccttgctttactaccattccgaactgactgaggtcatattttccatatattttaagataaaaaagccttagaaagtttctactgtcactaaaatgatatctaataactatcaagtcacaggcagtacatttttgtttcttcctaatggttccttaatgctaacttttattagcattaggttagtacttagaatacaaaacaggaatgaacagaggaatacaattttctctattccctttacctcttctttcattacaataacatttatatttggaaggtcagttgctgtaatgtcattactattttaacattaacctgcagaaacaagaatagctattcttctatcaacataagttctataaagtatcattgattcaatcacacctcattgaaaataattaattttgactgcccagaaaggttagcaattttccagcttcaattcaactagttttaattgccagtaggaagcctggatactcaacttgtctatatcactgccaaaaatggtgaatgagcaagtattcatcaagacctcagtacagtcggtgctttacacatattgacaggcacagagcaaggcacggaagagatatctgtggacagattagtaggagaggagatgatggatggatggaatcgggtgaacaggcaaggaggaaggaaaggcagagggtggcaagaaagaagaccagaagaatccctaaatccaagaagcatatataatatgatctagacagtaaagtacagagaaagtgagatgacagccagtaaggtctggctggaaaagatacaaagaggaaacggaattcaatcagcacttttcaaatgctaaagcaaaagagacgaatcatgtattccaagagggtaaagctgcaagacaaaggcacaaagtcgggaaaatgaaatgcatgttcataaaacagtgtaaagtagagcatttatgaaggagaccgtgggaaggtaaagctgcaaagtcaggggtttgcagccagatgctgtgcagagcagaaacactgagggtgtcagctaccgcctcctcttcccaccccttctttggaaagcagcaccctcccccaaatcttacagccacaccccatcatatgcttttcttatccaaccccccaccacagaatatctgataagtgacaaaatgcttacaattccagaaaaaaaaaagaaaaaataatacataaacaatttgcaaaaaaaaacaaaacaaaacaaaactggggctcaactagtatctggtcagttcatatcctgctaagttttgtatacaatttcagggttgttttaacaaatttggctctgatcactttgaacaatgactttttttttaagataataaatccagtcaggagagcaaatagccccagttaagtgcctctcatgtaccaggcagaattgtaggggctttatgtatgctatttcatttcatcttaatagtattaatttgaaattattcttctaccttcatagatatgaggaaaaattactggagactcacttagctaaacagcaaaactaggattgcaaatatacctagaataaacgtagttagaataactactgctaccttcatccaactgtatattttctgtatcttttcagaagaatcagaaggaaaagcactaaaagtacctgctccatttcaaatacgcatggcttatatccaaacgcctaaagctcttaaaactaggcccatcctcaactcataaagcagtcattaaaacccatccctgaagctactggtgcacttgacttcaatccaaaccacaggcctaaagaaaccatgagaaaggatgtttcagacccatctaaaatgattgaagatcagtgttctggtccccgcttacatcgccaaattcttctctgatatatggaacccagtaaggcagtatttaggggaaagcatagcattcatataaacccctttccttttataataagacattttatacgtatcaggcagattctgaccttcattcgtcagacattaactggacatctatactctgtcaggggctctgctggaaatcaaactactcctggccttaaaaaaatccaatgtagcaatttaccaagccgtatgagaaaatgtgtactcctagagtgggttcttccctctactgatataaaaatcaagataggcctcctagcacttggggaggccaaggcagatagaacacctgaggtcaggagttcaagaccagcctggcctacatggtgaaatcctgtctctactaaaaatacaaaaattagctgggtgtggtcatgggcgcctataatcccagctactctggaagctgaggcaggagaattgcttgaacccagtgggcggaggttgcagtgagccaagatcacgccacttcattccagccttggcgacaagagtgaaatctgtctccaaaaaaaaaaaaaaaaaaaaattcaagataacctttagaaaggccaaaatcagagttgcacaatccaagttctagagcctatgcactttattaattagctagtcctccaaaagctcaatgatctacttggtttcatcttatacatattaacaacagatttggttttctaaggacatatcctggatgggccacttactagcttaatgcccttagacaagaagttactttactgtctgagcctgggttttttcattcgaaaaatgaggataaacagctacacctcaagactgttgttaaggattacagatagttatgtaatgcaccaacagagtctggcacttagaagtacaataagtgtaaccaatatcataattacaggaattgtaagccaattgcaaattgatcagaaaagtcaaattacaaaaaaattgagggacaggagcaaagagactaaagacagctatagccgggcgcggtggctcacgcctgtaatcccagcactttgggcggccgaggcgggcggatcacgaggtcaggagatcaagcccatcctggctaacacggtgaaaccccgtctgtactaaaaatacaaaaaattagctgggcatggtggtgggcgcctgtagtcccagctactcaggaggctgaggcaggagaatggcgtgaactcaggaggcggagcttgcagtgagcagaagtcgtgccactgcactccagcctgggcaacagagcgagactctgtctcaaaaaaaaaaaaaaaaggcagctataacaataaatagctggcagcaagaaaacaatcaagaaactacacagacacaaaaactcagaaactacagactgaagccaacagcccacacacttcaatagctgttgaggacatcactaatgtttttataagatgtaacaatgatgtgcctgaagatgacattaagccctttaaaacagattaaacttttattccatgtgtttaaaaaaaaagataagcacataattgacttgggaaagattcctatcaaaagcgatttagcatcacaaatttaacacgttggatatctaaactttaattaactgcaaaactccagtaccaagcttctgttatacttaatatgtaaaggaacctcccgaatttcagacgccattttttctactaaacactgaaaaagatgtatttcaaacacattaattatgaaatacttaaatgcaatggcctaaggcatcatcagtttcttaatagctaaaacaagataaagtttggtctccctcattgctaaatgtttttagatctaccaaagagacacagacatgtgccatagaaatcccttttcacttgtattttctccctacatttggaaggcggtaaaaagcgaaaaacatctgtttcttcctgtaatgctgacaaccctcattctatggtttaaaaataaaagatgcttatggaagcttatagatataatgatcacctgcttcaaaggcagctggcaattaattgatccaaatacatgcaaaattaaggccaaaaagaaatttccctaaggacaatgccagtgttccccaagcctctggtaatttccaagacacccgtaaaaaaatagcagaggagagtaaagaagcaaacgtagtggcagatgacatgaagagttgacagctgagacacaagggcctgtgtcaatctggattcatttgaccaccataagaacagtttccatgagagctgcttagagaaaatctggttcactctggccaacaatcacccttcatccagctcaaggaaggccagatgttcaaccattaaaggaggacaccttattaaaaacaggaactggaaaatctaattaaagaaaggccctaaatctgatcacattttttataaacagtttttactctctatatggccttaaaacattgtatatccaaacagaaataataatgacattagacttgattcattgactcagtttctatttctggatctgcctctgaatgaatcacagtgtgaccttgagaaagttatttcacctctttctctatctacattttcccaccagttacaaagttaggtaaaacctactgatcctcttttcctgaacaatgctgagtgaatgacataaacatacggaagctgctacacaaaaatatatttgctgattaccaaccagctgtatccaaccatcaaatacaaaataggtgatgtccttataaaaacgactagctctgcaattttactttgcacaatttaaaaaacaaatcacctggagaggtcaacaaaacccaggtggttcctatacaggactcttttaagagtcccaaatttcaaatttgggaatacttagcaaattaaactaaaatgcaagttccaagaagacacttgtttttttaaaggaaaacaaacattcaacctatttagggtcttgtgatcttccaggactctgttcaatcctcctctctcaagtcctcttccctcaccccttgccaccaagcagataagccactgcatccgtgatgttcccagcagtgatttgcatgtctatatctatacagatatttctgtgcactgcacttatgtcatgtctcctcctcactagtcagcgagccttagaaaggcaaaatcatgatttaatggtctatgtatcacctacatctagaatactgcctagcacagtgtgctactatttagaatactgcctagcacaatgtggctgctcaatgtgtgtgtgtgtgtgtgtgtgtgtatgcacatatatgcttacaaatgtatatacatatatatatttgctaaatgaatgaaataaataaatttccaaagtcttatgctagtaggaatagctatcattaatattaatgattgataatcattatggtgaattattttcacttttcaaaaactatatacgtgaatgtcacttagataaagagaaaatatgtgatttcttaccaacattaaattcacaaaaattaatgcaaaaactttccaatcttcttctatcaaaagtgatctttcttttttatttgttttgcattttgtttttgtttttttacaatttatctagtgggtataaaactacaaacattctttttaacaatctacacaataaagagaaggtggaaaagaagaaaaataagagtatgtgaaatgaatggaagaagctacttttaagggtaagaccctataagggcaaaataaaaggtggcccacagatgaaaattaaaatgtggtcaataaaagtagctaccccaatattaccagcatttagtcaactaagaagtcgctgtctgtgccgagaaatttcatctaagcaatgttttacagataatgtatttattaaaataatagaacataagattttaaaattttcagattgcatttgaaatgtttaatccttcaaatatttatcataaacctatcagtagtcatgtaacttttcatatttttcgattcattaaattctaacactctatttcatttaataattaagaatagaggttaggtaaacatgttgggctcttaaggtaaggccttgggccctgtaagcctgtaagctaggataaactcattggactgcttgacactgaccagagcaaacatactctggctgtgctttatgccgccctgggtatgagtctataacatatgctagggaggggaggtaccagttggactagacgtgcttttgacctttccaccttcactgtctccaaactacaggctgttctgggctggaccatgaaactgaatgaaaattatccctactattctatttatttattaattttctaatagcatgtatcggattttcttgcagcagcaaacagcagtgagtgaccgcttaacttcactaagcagcggaacccagactgctgcaagatagaaactacatatcacatacataccaaacgtggacaaatacacacagctatgagagcggtcagagactgcctttggatcgtaccggaataaatgatttgtgcctccaaaaccgactcttgcgaaagaaccatgaacaattttttttctttttaatgctgtggtgatgaaaaatgaaagagaagaatgatggtttagctaacgagacgaaaaaggagtttatctccctggcgtgacccgaaactttgtgaatagcaggtattttggtgaaatgggcagagcaacttaggacagacacaactaagttgttccctttaagatcttgtaagaacacagaaaaccaacaagtatgtccaagtcatttcaccgtggggggtgagctcgctgggcggaaaaggattccctacggagaagagatgtgccactaccaggtcaccgccttccagagcccagggagggcgggtggcaggggaaggccctgactgacaggcgacttgagacccggaggccagcagcagggcgcgctcacctgtgaacacgttctggaagccatccgcagccgcgggggccccagacgccttggcggccagtgctggggacaccccaccgccgctgtcactcgccaccccgcgctccagcaggtgcgcggagcctcccacccggggcgtgcgggcgcctagctcctccgtccagtcagccgagcggctcaggccggccgcaaggccgccgtcggcccggtgcttcttggccggggcggctacggaggccagggcagttctggaccctcggctcagggagggttccggccgcctcttgatcctggggctctgattaggggactgagacggggagtgattgggcgagctgagctgccgggtggtggtcttgatgtagcagggggtgatgagcgggtagggcttgaaacagcgctggctaggcgcggggctgccgggcagggaagcggccgcgggggcgtcaggtccttgcgcctcctcctccggctcttcccccagcctctgcggggcgtcctctcccacctccggggcccactcctcccccggagagccccggggcgcatcctcaaaagcatcctcctcaccctcctcatccgtgtccccagcccctcgcacgggggctccggccgcttcctcccccggcccggcctcgggaaatgggaaagccgtggaggagggcgagtctttggccgcgggttgcgctgccgggagactgggcgcctcggagaccgggaggccgccgggggacggcggttgctggggctcccggggctcggcggccaggctctcgggcaggtcggagagcgcggacagcgcctgctcggtgtccggactgcccggggcctccccagccccgccgctcggccccagcaggaaccggtccaggcccaggaaggccccgggctgaggggagacggcagtggggggcgctgcaggctcctcggcgccctggagctgctgctgctgctgctgttgctggagctggagctggagctgctgctgctgctgctgctgcaggcggatcgcctgctggatgtctgaaagcaaatcctcttgctccgtagccgaatggaagctatagatgtccgtgtccgagcccgagctggtcctttgtccatcctgcgcccctgctgcagtttccacatcctcggcgatcggccggcccccgaccctagcctcggcaggcccaggaccccctggaccggtcacctcaaaagggtccgcacactcggtatccgacaggccggcctcgtccgccgagaggctgaggtctggagtcttggtgagcagggagtgagcgctgtccagctccccggtctgcagggcctgggaatccagtacatcttcgcgggagccgccggcgcctttccccttggacagattcttcctgatccgcaggttggaaaacaccgaggctctggagtcggacttgctcttcttcttgcccgactccccgccgccgccgccgccccctccccccttgccgtgcttgcctagcgccttcttgcccccgctccccttctttgtggcttccacatccctgggccccagcgcatcctcggcgccaccgccgccttcgtgcaaagcatcacctgcgctcctcttcagcttcccatcctggttccccatggtgcaatcccgctgctgcgccgccgcgccgcgggcactcaggccatcccggccctcctcggccccaggtccgggcctagggagtctcccgggagagtcaggcggcggcggggggagccgctgggaggggaggccgacgcgcgcccggctggccccgctcgcatctgccgccgctttgcataatgcgcggcggctcccgtggctgccgtcgctgcggggnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgtttttttttttttttttttttttttgagacagggtctcactctattgcccaggctggagtgcaatggcacaatcacagttcactgtagccttggcctccagggcctgggtgatcctcccacctcagccacttgagtagctgggaccacaggcatgcaccaccatgtccagctaaatttttttttttttttttgagactgagtctcactctgtcacccaggctggagtgcagtggcacaatctcagcacactgcaacctctgtttcccaggttcaagcaattttcctgccttagcctcctgagtagctgggattacaggcacgtgccaccatgcccagctactttttgtatattttgtaaagacagggtttcgccatgttggtcaggctggtctccaactcctgacctcgtgatccacctgccttggcctccaaagtgctggaattacaggcgtgagccgccacacccagcctgcccagctaaattttgtaactttttttttgtagagatggggtcttgccatgttgtccaggctggtattacaggcatgagccaccacgcctggcctctcctattaattcgacttttgtcagctgattttcagtgaaccttcagagggcaaaaaggaagtattatggtggcctttacacatatttcaaagtgtttgcctatttgcttgcctgtctctctcctctagatttcatacctctgaggatagggcagtgtcttatttactattatatctctaggaccccacgcagtgcctggcgcatagtgggtatgccttaaatgaattgatatatctcccatttgcattgtactaatggtgaactgacaagaaaagctaatatattcttatggcccctacctccagcaattctatataagaaggtagcaggaaaacaaatacacagagagagaaaagacgatatggttgaccccaagaaagttcagtggccaaaaatctaagtgcaatagaagtttagagagagaggatgtcccgcttgatggaatggttggaggagactgaatgaggagcttaaacctccacattttctcctcactacttttacagttcaggacaaaaggcactttttgagcccttgggcaaggcagggaacaatttatttagtgtcggtcttttcccagactgggagccatcattcccaagaggagtttggcagtctgtcctctgaccttcatttacaaggagtccaaagcttataattactattgattgctgttatttttgcctttagtttatatctgatcacaatgaacaaagacagatacagaatttcagattttggaaactgtatagaagcctgcaacaagtggatgacaaagaggtcctccaaaagctagtttgctagttgctctagttgaaaaatatataatagtgtctgctttcaaaaagatttaaagacatacaatttctttcaagtcttttttttttcttttgagacagaatcttgctctgtcacccaggctggagtgcagtggcgcgatctttgttcactgcaacctccgcctaccaggttcgagtgattctcctgcctcagcctcccgagtagctgggactacaggcacgggccaccacgcccggctaatttttgtatttgtagtagagacggggctttaccatgttggtcaggctggtctcaaactcctgacctcaggcgatccacccaccttggcctcccaaagtgctgggattacaggcgtgagccaccgtgcctggaccctcaagtctcatataccatacaaagcgaaacaactgaaggagtctaagaatcaggaagcgttatgcttaaacacagttttaaaaagactttcagaatgttgacctggcacttttataaaaaagttcaaatttcctgatagtagcgcttactgttactatttatttatttatttttgagacaaagtctggctcaggctggagttcagtggagtgatcagcagtcattccaccttggcctctcaagtagctgggactacaggcatgtgccaccatgcccagctaatttttaatgtgtttgtgtgtgtgtgtgtgtgtgtgtgtgtgtgtggagaaggggtctcactatgatgcccaggctgatcttaaactcctgggctcaagcaatcctcctgccttggcctcccaaagcgttggaattacaggcgtgagccactgcacccggcaagtagtgcttatgaaaataccataacaaagatgtaatttcccacatgcgaggaacaattttggttctgctatcactgttccctttgaccatcaggcaagagtatgactcccgctcatgttggccttatttacttcaaagtaaatgttaggtgtgtttatttaacctatattgctgtgaattccctgactattcctgttcaaaaaaggaattcagaaaataataaggtttcagatgaaatctttcaaggaagaaaagagaattgctgtacatgagcagaaacagctaagagtatagcatgccgacacaaataggaacgcttacaagactagaaagccttctggggaaatgatttgggagactgtggtaaggaggtttatgaatgttcgtgggaggcagctccgagcataaaggtcaataaaaaacaagggatggagtttgggagtgggtgaggaagacaaagagcgcagtcagaagatgggcttggacagagtagccggcgagctgcatgtaggagggtagagggtgagaaatgcactcagatgccaatagaatcagctctatatgacgcaggagatgccaagaaaagaatgcaaataagaatgtagcaaaacatgggtgaaattgttggagcaggtggtctctcgctaaagaggaaggggtagggggaagcagtgggagtgaagtggggaatatgtcaccatacttacaatgcacaaaaatgaaatgtgtacattgattttgtaagatcaaggctgtgtattctttcttctaagagacactgtcaccgaggctagagtgcaatgtcacagctcactgcagccttgagctcttgggttcaagcaatcttcctgcctcagcctcctgagtaggtagaactataggcacatgctaccacacctggctaacgatttgagtttttgtaaaaacaagtgtcttgcttattgcccaggctggtcttgaactcctagcctcaagtgatcccctggccttgacctcccaaagcgctgtcattacgggcatgagccactgcttcttgccaatgctaaaggtgtttaagaagagcagtcaattaatttataatgttgaagaaatatcacaaagtttgcaaatgagaagcacaaggattattttttaatgccagtaataagttggatgagattgaaggcgttactaaagactgaaaaaccatatcaagagagaagaagaattgtatacaaataaatagaggaaatatggtagcatatgctatgtagtaattttggaatatatgaaatatattagttgaactgaaacggttaaaattataattattttgcatatctgtgcagcacacatttaggtttgacaaatacaacatgggaatattaaatatgctttggtatcaaaatacaaagaagtccattatagttaactaaatatgtcttgctaaaaattataatgcaaatgagcgttcttcatcgctttgactgcaggatcggatatatatatagataattcatattcaaaatctcaaatgcagccacctgagtggataagaagagaagtaaatattagttctcctctcaatttatttgcttatgaagaacgaggctgaaaatattgaatacatttttatttttctactgtgcttttaaaagggagttagtttaagagcctaaggggttgaaaaaaatatattgactggaaacaaatagagatattttttcctggaaaacgtatgtcataatttataaaacaagtattgggtttttttgtttgtttgttttacaatgctgtgaaagcttacattcagctgatagtgtttaataagtgtcgttttcagacgttattatagtgtgctgataatcttggcaaaattcatgagaactgtttaaatgaaacaaatgacagagatgattgtgagttcttgaaaacacgtttttgggagggagctaagtgtttaaatctcttcagtgagctcttctcctagcagcttggcagttgctaccttggatgtgttagagacagatgctgaagccaactcatggttaatgaccttattcaaggtcactgaataaatcggtttcagtcctagggctagacttctaaccacagagccattatgtgcagcctgtctcctgagctgtgttgccattaattgaccctttaaatacgttgggttttcaaatctcatttaaaaattcaacgtgatgaaaccataaaaatggactctccctctctctccctctctctttctctctctgtctctctttctctctctctcacacacacacacacacataatttatctactttgcagcattgtttagggctgagtgaaaatccagaagctttcagatccagacaaatccagcttatagaaatgtgttgtttggccaacagaatgtactcactttggttttaaattagctgtcaacatttagaaattgggagtttggtgtagttggcttcctgacttctttttaacaatttaaagatctggggacattggtttgtcttttcaaatggtaaaaatcccctgcagctgggtagcacaaacccttttaaacgggcataattcatcaccatctccacttgggggcctcttcataatttacattaagtgtccagcctctgtaggcatgtgatctgggagggacctaattatttgaccagattagaaacattcaatagtttccaggccattttccagggtatgccagtcaaagccttcataatagatcttcaatttatctttccatcttacctttctgcattccccaccaccatctttctgtttccattcctgaatataaaagtacacttgagtataaagtatactgtctttatctaaggtactcactgtgtcctaagtacaatgcatacatgctggtattcctgattttatgtgtactcctggcttcccttgcttaaaaagccttccctatccctacaccccagggcaacctcctgtggaatttactatcagctctaatattccttcctaaatgtagacttgaaaattatctcccatctccatcttctctggggttccctactttcttctctttgatcatctatcacagcacttagcaacttgccttatggtaatttggctacttatccatcccacactgtgttatttgcagctctggcctcaggatctgactttccattttctcactttcctacaggagcaaagttgtggccaggaccattgacagtccagtagttcgttctgggcttaggtagaggtaccctgcagttagctggggtggtccgaagaggcttttgctgagcagactcttgcttgaagatagaggtaattgcttgaagggaatgtatacaccctttctgattcttgatcttccttaatgtggagataggggatgaagacaggagtatagtagagttgctccgtataagacctgatcactttccaaaaggggcaggaccaccaggcagcaaaactgtatcatctgtatcattttaatttaaaaaggagagcacagttctggttcaagagccatcaaaactgcttctgggcaatatcacaatgtgtcgatcataaggagttacaggccctgcttgtagcctttgctgaacccaaaaccctaatcaaaatgatgtgtgttggtttccagccaagtcttgcctttgaatctttactgaaatagattaagggaaattgagatgtcatatagacaaggtcctggtcaatgagctcagctataaatcaaaccaaagcactacagatagaagccaggtgtataacccatatctcaaagaaaatcaacctccttccctgcaccccctcccaacctcgaggaacttatatggtccttcatatactgcccattttgggcactgtgccttgatgtttctgttatccaaaaggggttccgattcagaccccaaggggtggtttttgaacctcacgcaagaaagaattctgggcgagtccacagagcaaagtgaaagaaagtttactaggaaagtaaaggaataaagaatcgcttctccataggcagagcagtggcatgggcggcttgagtgaatatacttatagttatttattgatgatatgcttatgctaaacaagggttggattattcatgagttttccgggaaagggcaggcaattcctggaactgagagttcctcctccttttagaccatatagggtaacttctggacgttgccatgacatttgtaaatagtcatggcactggtgggagtgtctttgagcatgctgatgcattataattagtgtataatgagcaatgaggatgatcagaggtcacttttattgccgtcttggctttggtgggttttggctggcttctttactgtgtcttgttttatcagcagggcttttgtgacctgtatcttgtgctgacctatctcatcctgtgactaagaatgcctctgggaatgcagcgcaggaggcctcagccttattttccccagcccctattcaagatggagttgctctagttcaaacgcccctgacgtttccatgatttcactgtttctgaggaagcattcactgccaagtaacactgcgtgtcgccctcagctcttaagatctcttagtacacaatactatctttagctcttggaatattgaccaaatgcaacattcattagaaggatgttccagatcagaaacccaagtggtaattggatggtaattccaatggttaattgtgatggtaattccatcacactaaaagtacaactgtgtcctttccattgtaattaagattcataagtaacgatcttctgaaagcagtatattagtttcagcatataattttaggatgcttcctataaaaagtatctgagtgtaatcccagctactcaggaggctgaggcgggagaatcgcttgaacctgggaggcggaggttgcagtgagccgagatcatgccattgcactccagcccaggcaacaagagtgaaacttcatctcaaaaaaaaaaaaaaaggatcagagtacattattgaacgttattgaaagtagttaaaactcacatttttaaaagtgaagataaggcatcctatggttgtttaaatcagaagtgccatattggtatcagggacttgggtgtattctatattctaattttgctgtcatttgacttgtcccttcctggtttcaagacagttgttacacttcaggaatcagacaaagacaatgtcttacaaaacaaaggttaattatataagatcttttttttttttttttaaactagggagaaaacccaatctaataagtctcccagcaatctcccaccaggtcacatgggccacagttagatctcactcttagcacaaactggaagaaggaatggaaatagaatacacagcattttcagtcctatacttagccagaatggaggaggggatgtcggagtgatgcagggaaaggaggaaatggctattttgaaggcaatcaacaatgtctacccatcctccaagaacgcaaagaatcagcgcaaagaataattggtcattcagtgtctttcaatgcactacctatgttctgaatttttttggagcggggagggggatggagtcttgctctgtggcccaggctggagagcagtggcttgatctgggctcactgcaacctccgcctcctggattcaagtgattctcctgcctcagcctccccgcccaagtaactgggactacaggtatgtgccgccatgcctagctaatttttttgtggtgtgtatttttagtagagatggggtttcaccatgttggccaagctggtctcaaactcctgacctcaagtgatccacccgcctccacctccgtctcccatggtgctgggattacaagcatgagacaccacgtcctgcccgatgtttctaatattaaaggcctttcactaacgtcatttacattaagtgcttgtttcttacctactcatattctgatctgatttctgtctaactttaaacactagacaccataccctgacttcaatcttgctattgtctacttctatttgatgtttgaactaattactcgaatgtttcactcccaactctttctcatggactttcagtttagccttagatttctgacttctgtactccagattactttgatgggtgacttagtttgctgggaccccacagttagcattggctgcctgacaagagcagatggatttcacatgccctcccatccccaacagtgtcagcacagagcatttgtaccaagacaccaggtggaaaacaccatggaaaacttgggaccatcctcttttctgtttccacctgtgataagaattacaaagatactatttagggacaaaaggattctaaatattataagaaaagaaacttacaataatagaaaagcagacaattgtttccctacccctacacttaaaacaacacttcgggaggctgaggcgggttgggtcacctcaggtcaggagttcgagaccagcctggccaacacagtgaaaccccgtctctactaaaaatacaaaaattagccgggagtggtggcacacgcttgtagtcccagctactcaggaagctgaggaaggagaatcgcttgaacctggaaagcgaaggttgccattagctgagatcccaccactgcactccagcctgggaaacagagcgagactccatctcaagaaaaaacaaacaaacaaacaaaaaaaaccaagaaggttccacatattctgtgactccatttctataaaacgtacagaatagggaaatccacacagcagaaagtagatgggtggttgccagggtctgggagaggttgtggggaaatggggagtgacggctaatgtgtacaggtttattctggggtgataaaaatgttctaaaattggccgggtatggtgactcatgcctgtaagcctagcactttgggaggctgaggcaggtggatcacctgaggtcaggagttggagaccaacatgatgaaaccccgtctctactaaatacaaaaaattagctgagcgtggtggtgcacacctgtaatcccagctacttgggaggctgaggcaggagaatcgcttgaacccgggaggcagaggttgcagtgagccaagattgcaccattgcactccagcctgggcaacaagagcgaaattctgtctcaaaaaaaaaaaaaagttctaaaattggttgtggtggtggttgcacaactttatgaatataataaaagctattgaattgtacactttaaatgggtgaattatatagtatataagttatatctcgctaaacctgttttttttaaaaagaacttaagattttgaaaagtgttattcattagattccagtggtgtgattgcagcctcgtaatccacttcaccagcttgcaaatgcttgacacattactaaagaaaagatgaggatttgccgatactctggtgctatagaagtatttctattcttcagtgttcaaaccaaaaactgtgaatccgctggatgagtaggaaaagcagaggacataatgttcttcagttagacactgtctttttttttttttttttaaattgagacggaatcttgctctattgcccaggctggagtgcagtggtgccatcttggttcattgcaacctctgctgcccgggttcatgcgatccttgtgtctcagcttcctgagtagctgtgagtacaggtatgcgacagcatgcctggctaatttttttgtattattagtagagatggagttttgtcatgttggccaggctggtctggaactcctgacctcaggtgatcctcccacctcggcctcccaaagtgctgggattacaggtgtgagtcactgcacctggcctagacactgtctttaacagaaatgtacatgccactgtcccccagactttggtcgttattgatgtgattgctggaacataatgcttcaaattatccctcaattcctggactaaatcaatggttttacatgattatcactgaaacgtaatccattaagtgcttaatttacatcatattaacaatctcagtacactgggagtttctggtttgtgacagatcaggaaaaatatcacttcaaccaatgcctgtgaaacatttttccttgagtgcaaatgaatgtttcagtctagtttttgtatgtgaacatcatggtaaccaacttcaggaaaaagtttctatcctttttttctaacagataatactattttcaacatataaaaggaatgtattatttctagggatacttcaggtcccagcagcttctgagtcttctcaacaattacactaggtaagtacccttggatatccacatttgcagcttcaagttttacagattaaatcgttctgatagttttaatgctttgtgcactaggatgcaaagatgctaatttttcactgcatagagtagcttctttaattttttatgttctctattacattataagaattgcatattatttatttattctatgcattggccccattcttcttttctttgcccgctcaggttttctgttttgcctcctggtattccttcggctgcattcacctcagtttcccttgctaactctcctttgcttaaggttactttttttttcctttttttagacagtatcttgctttgttgccaagagtgaagagcagtagtgctatcatggcttactgcagcctcaaactcctgggctcaagcaatcctcccatctcagcctcccaagtagctgggactacagctactgatttttaaattttttgtagagatgggatctctctattttgcccaggctagtctccaactcctgccttggcaatcaggcaatcctcctgccttggcctcccaaagtgctgggattacaggtgtgagccaccatgcccagcccacttagttttaaatactgctgttatatgtcatttcatccatagcttctaactctgtacactctctattgaggaaagctgggctttttgtttctgtacattctccatgccacctagcactggtaataacaatattaatcacaattcacatttttggagaacactgttgagtttcatgagcctatatcatttaattgccaggcttgttaggcagatattgtactgttatgtatcttatacagaatgaaacggggtttagcaagaataagtaatatcaccaggatcacatgattagcaagtgttgggcctgggcaatcagactccagagcttagactgaaccactgcactgtctttttcctgataaccaggcaataggcttcacttcttagcctcaaagatgaatggaggcttggcatatcagaatatttgcatatcccattttttaaaaatagaggcagggcgtcgctgtcacccaggctggaatgtagtggcacaatcatggctcacagcagcctcatcctcctgggctcaaatgattctcccatctcagccccccaagtagctgggactacaggtgcgcaccaccacgcccggctgttttttttttttaatttttttaaatttttagtagagacaaggtctgtctaaattccccaggctgatctcaaattcctgagctcaaacgatccacccaccttggcatcccaaagtgctgggattataggagtaagccactgcgcccagcctatacctcacattttcatttaaatttgtaatcattctagatcttgccatcataaatttgaattatggactgctgatacttttctagaatattcagatatttaatctaattattctgaggactactgatttataatgttgagatataaaggtttgttatagtaatttacatttagaatatttgcaatggaaaactgtgtttgattacttctggttctaaaaagtattttttataggaagaggaattgccaccaatcttgttatagttaactatactatgacttctttggtcttggtaaaataaaatcaattgcaataaaagttatgtgattttttttggagtcttcattttctatcctttttctctttgcagattctttttaggcataataaacaagtagttctaatagaccaattttaaagcacaatgcaaaaatttattcttttaggaagtacagcataccttagagatatcgagagttcagttccaggttccagaccaccacaaaaaagcaaatatttaggtaaagcgaatatcacaataaagagttacgaatgctgacacagagacacgaagtgagcacatgctgtgggaaaaatctcactgatagatgttcgacacagggttgccacaaatctgcaatttgtaaaaacccataatatctgcaacgtgcaataaagtgaagcatgataaaatgaggtatgcctgcaaaccaaattgttagcattccctaaatacatcaccctctatttctctgtgtctttaccacacacctaagtccttttcctataggatcttacccccaccctatctctggtaccctccctgtgcttgctgaattcctccttatcctacttaccttctgtggctctcctaatatcacttcctgggcgacatctttcaagacccttgcaggagaagttagtgtttctctaatgatttcatggcactttgtccaaatctgtaccaaagtacttatttcattgtactacaattacttgtttatatcttcctctgtcattgtggactacttgagaggggctatatcttttccactgtcacaatatcatatgtgctcaggaagtgagggaagcagtgagcgagaaacatccacatgaagggcattgaagggtaatgagaggtgatgagataggaaacctggggaaatgcctaaattagcattttgctttttgccatgtacacagtattctacctggagaaaaagcagtgaaaggagtaaggttatggttagcccaattagaattgaggaagaaattcatatatggtttactatattttttgaatattccatgttgaatgtatttatattgctttgaaagatgattcctttccttgctgaaaatatctcatgtgatattttaaatacattctgtgtggactttaggcataccctgagactctcccaaaacctttgacataaaatatattttctatgaagaaagttagtgaattgtatgaaaagttatcatccattggaattattactggcaaatgaaatgctattctgcggtaaaagattttttttcatttcaaagtattgtatatttcacataatagacaaattttgatagcaagatcacattttagaaggctgtcatcttctaataattgtgttaatacctgccatattgtcttagtttgtttaagaggctaaaacaggataccacagactgcgtggcttaaacaacagaaatgtatttctcgtagtcctggaggctgggacgtccaagttcaacgtgtcagcagattcagtgtctagtgagggtccacttcctggttcatagacagccatcttctcactgtgtcttcacatggtggaaggggtgaagaagctctctgaggtctcctctaccataaggacattatgtcattcgtgaggactctaccttcatggtctaatcacctcccaaaggccctacttccaaataccatcacactggggattaggcttcctcgtctgaattttgaaagacacaaacattcagactctagcagatattgacgttatttatttcccctgggtttctagattatcaaagcctgatgtatggtggggggagttactcagatcatctcagtacagcttgccatatttttaaattaaggaggtaattttagaacatgggtgcctgtaatcccagctactctggaggctgaggcaggagaatcgcctgaacccgggaggcacatgttgcagtgagccgagattgcgccattgcactacagcctgggcaacaagagtgaaactctgcctcaaaaaaaaataataataataaataaaataacatatgaaaaactctgctgaggtaagaaaaggtaatgacttttccttaatttaaaaataatcagctcttcatctcccccttaaggggctgcttggagggtatttgtgtttgggaaaaatcagaactttcaaaccttcttttagtgaaccagagttgtgagagtaattacatgaaagtatggaggtagacatcttgagtaaagggttttgtacctctagcaagtttaggtgaagtgggccttgaatgaacgtgtgatttctaaagcttgagaagtcattgataacattctgtaaacagactttggagtagcagagcattattttaatagctaaaggtctatcgctcctgcactcaacgatacaattctgagtgggcccgtgtcctaatatcacaactactctaggctatagagcaactttgaaagttctagaacttttgaactttcacatttgaaccacagaatggctctctctcatttttcccttacttttctctgctgtttgaatcaggggtcagtcgagtctttctataaagggccagatagcaaattaggctttgcggccatctggtctccgtctaagctgctcagccctactattgcagtgcgaaagcagccatagacgatatgtaaatgaatggcatggctgtgtgtcactatcagtttctttataaaaacgggcagccatcttgaagtcatatagcttgctgactagtggtttagaagagctagaaagaaattctgggctgaggtgtttcgggtttttatgtttatttatttcacatatataaacgtgggcatggggaaatctgtgatccagggaaatgttctttttcttcctctgggtgctggttatacaggaattttaactttgctaaacttcaacaagcttacaatgatcatttgtgcatttttgtatatatatgctatattttaatttaaaaatctattttaaaacgttttttccagtttttttctttttcttttaaaaaatgtcttaatagtgttctagctttctgcttccaaacatttctagaggaccataaccatatgcaaagttcttttcttttctttttttttttttttagaattccatttagacattttattccttgaagcatgtaaaagtacgtaaacaccccaaaaggttatatcagaagtcaaagcattgtgatctctctggatgcttttcagacatcgtgggtgagcgttctgaatcccctgtccccactgccccggccagctttgcctggagagctactgagattttctaccatctagtcttgggatgctgaagcctcaagaggttaggaatgaatgcaattctcacccacctggcaatgttggtatgcaagctttggttggtatgagaccacctcatcctattgaagttcttttcttgtctttcaccatggaatcatttacttgggaattgaacaatgagaacacatggacacaggaaggggaacatcacacactggggactgttgtggggtggggggagaggggagggatagcattaggagatatacctaatgctaaatgacgagttaatgggtgcagcacaccaacatggcacatgtatacatatgtaacaaacctgcacattgtgcacatgtaccctaaaacttaaagtataataataataataaaataaaataaaataaaaaaaaagaagtgacatgcacctgtactcctagctgcttgggaggcttgaggactcctttagcaaagttcttttctagacatagttttatttcagctttggataagcctgttttatttctttgcttatttggacctgggctgagttttgaactcccccacaaaactttccatgttatccatgaaaagacgcatcatactccagagaccagccttctttctggctgtcacatttcaccagaagtttcacaactgcccaaattcccacccactcttggaaagtctgtcacatgttttactcttttacgacttcgtgtgaacatgtaaatgccaagccaagcaagaaccctgctacccgccccttctttcaagcaaaaaacaaaaacaaaacaaaacaaaacacagggtgtgaaaacccgtgttctagtttttgaggaatttcaaagtggccagtgggatggagtatggaattttagaagaaaatgactcatcagaattttatttcgagaagaagaaagaagcttttctcaatgccctgcatcaggcgcagtcctgagattgttcaccatttttaccttcttcaatttgagtccaagagctttttaaaaatatacgtcagctagcagagcagattttgtgcactttttccaattatgctgttaataattttagaatttggaatggaagattttagtgcttccgttttagtcaccttgaagaaaacaaagcagcgtgtatcaaaattctaagaagtatgcacatcatttatttccaaaattaattcctaggaattcatcccaggaaattagggaattgttcaaaaatgtatgtggggtgatagccatcgaaaattgttcatgcgatggctcacgcttgtaatcccagcactttgggagcctgaggcgggcagatcacctgaggtcaagaagtttgagaccatcctggccaacatggtgaaactctgtctctactaaaaatacgaaaattagctgggtgtgatggtgggtgcctgtagcctcagctactcgggaggctgaggcagaagaattgcttgaacccaggaggtggagaggcagaggttgcagtgagctgagattgtgccactgcactctagcctgggcgacagagtgagactctgtttcaaaaataaataaacaaacaaataaaaataaaaaagatttctcagatataaataagtaagaaataacataaacatccaataattgtggcttatttcaatacattagggcccattcttctatgggatgactgtagcagccactcatgagtagtgcacctctatgtctattcatggggaatatgtggatgctgcatttcctctccagtcaggactcagatttcaagtaactccaaacccaaatcaaagggatttaatcaaaatagattctttcatgcaaatgaa

SEQ ID NO: 8 - protein, 11 aa, Mus musculus
  VARIANT 1: Xaa=Met or Val
XGIPPPPPLPG

SEQ ID NO: 9 - protein, 11 aa, Mus musculus
GMGIPPPPPLP

SEQ ID NO: 10 - protein, 22 aa, Artificial Sequence
  based on Mus musculus
GMGIPPPPPLPGVGIPPPPPLP

SEQ ID NO: 11 - protein, 9 aa, Mus musculus
IPPPPPLPG

SEQ ID NO: 12 - DNA, 24 bp, Artificial Sequence
  based on Mus musculus
ggctaggaagcagccgatcgagcc

SEQ ID NO: 13 - DNA, 20 bp, Artificial Sequence
  based on Mus musculus
acccgctcggagaagttggg

SEQ ID NO: 14 - DNA, 21 bp, Artificial Sequence
  based on Mus musculus
gtctgcagaggctgtcaatcc

SEQ ID NO: 15 - DNA, 23 bp, Artificial Sequence
  based on Mus musculus
ttctggaaagagggacggcagcc

SEQ ID NO: 16 - DNA, 26 bp, Artificial Sequence
  based on Mus musculus
cagcatttctggtccctgtagattgc

SEQ ID NO: 17 - DNA, 20 bp, Artificial Sequence
  based on Mus musculus
gaggtaaaagaaatcatggg

SEQ ID NO: 18 - DNA, 21 bp, Artificial Sequence
  based on Mus musculus
cttgaaaatattaagtgaagc

SEQ ID NO: 19 - DNA, 26 bp, Artificial Sequence
  based on Mus musculus
gtgcctgagcatcctccgtcctcagg

SEQ ID NO: 20 - DNA, 26 bp, Artificial Sequence
  based on Mus musculus
gaaagccatcagctgtcgctggagcc

SEQ ID NO: 21 - DNA, 19 bp, Artificial Sequence
  based on Mus musculus
ttttcctctgaacctcttg

SEQ ID NO: 22 - DNA, 20 bp, Artificial Sequence
  based on Mus musculus
aacgaatacatcatcctcac